(19) United States
(12) Patent Application Publication
(10) Pub. No.: US 2024/0382610 A1
(43) Pub. Date: Nov. 21, 2024

(54) NECTIN-4 ANTIBODIES AND CONJUGATES

(71) Applicant: Tallac Therapeutics, Inc., Burlingame, CA (US)

(72) Inventors: Pavel STROP, San Mateo, CA (US); Mingrui AN, Foster City, CA (US); Maria José da Silva Teixeira COSTA, San Francisco, CA (US); Hong Iris WAN, Foster City, CA (US); Maja Zukic BONACORSI, Monte Sereno, CA (US); Amy Shaw-Ru CHEN, San Jose, CA (US); Bang Janet SIM, Brisbane, CA (US); Min LI, Dublin, CA (US); Tracy Chia-Chien KUO, San Carlos, CA (US); Emma Ruth B. SANGALANG, Oakland, CA (US)

(21) Appl. No.: 18/444,564

(22) Filed: Feb. 16, 2024

Related U.S. Application Data
(63) Continuation of application No. PCT/US2022/075236, filed on Aug. 19, 2022.
(60) Provisional application No. 63/255,318, filed on Oct. 13, 2021.
(60) Provisional application No. 63/236,809, filed on Aug. 25, 2021.
(60) Provisional application No. 63/235,656, filed on Aug. 20, 2021.

(51) Int. Cl. A61K 47/68; A61K 45/06; A61P 35/00; A61P 37/04

(52) U.S. Cl. CPC A61K 47/6889; A61K 45/06; A61K 47/6807; A61K 47/6849; A61P 35/00; A61P 37/04

(57) ABSTRACT

The present disclosure provides nectin-4 antibodies and oligonucleotide conjugates thereof. Also provided are related methods of preparation thereof and methods of use thereof, including therapeutic uses.

### CROSS REFERENCE TO RELATED APPLICATIONS

This application is a continuation of International Application No. PCT/US2022/075236, filed on Aug. 19, 2022, which claims the benefit of priority of U.S. provisional patent application Ser. No. 63/235,656, filed Aug. 20, 2021, U.S. provisional patent application Ser. No. 63/236,809, filed Aug. 25, 2021, and U.S. provisional patent application Ser. No. 63/255,318, filed Oct. 13, 2021, the contents of each of which are hereby incorporated by reference herein in their entirety for all purposes.

### REFERENCE TO AN ELECTRONIC SEQUENCE LISTING

The contents of the electronic sequence listing (186492000601SUBSEQLIST.xml; Size: 1,499,371 bytes; and Date of Creation: Mar. 21, 2024) is herein incorporated by reference in its entirety.

### FIELD OF INVENTION

The present disclosure relates to nectin-4 antibodies and conjugates thereof and uses of such, including therapeutic uses.

### BACKGROUND

The nectin cell adhesion molecule 4 (Gene ID 81607; UniProt primary accession Q96NY8; known as nectin-4, PVRL4, LNIR, PRR4) is a type I transmembrane protein of the nectin family (which includes nectin-1, nectin-2, nectin-3, and nectin-4). Nectin-4 was identified in a bioinformatic screen for sequences similar to the nectin ectodomain (Reymond et al., Nectin4/PRR4, a new afadin-associated member of the nectin family that trans-interacts with nectin1/PRR1 through V domain interaction, J. Biol. Chem. 2001 Nov. 16; 276(46):43205-15). The protein contains two immunoglobulin-like (Ig-like) C2-type domains and one Ig-like V-type domain (IgV). The nectin family regulates various cell functions, such as proliferation, differentiation, and migration (Miyoshi and Takai, Nectin and nectin-like molecules: biology and pathology, Am. J. Nephrol. 2007; 27(6):590-604). Specifically, nectins are cell adhesion molecules that participate in adherens junctions and tight junctions between different cell types. Nectin-4 has also been shown to promote anchorage independence by driving cell-to-cell attachment and matrix independence (Pavlova et al., A role for PVRL4-driven cell-cell interactions in tumorigenesis, eLife 2013; 2:e00358). Nectin-4 has been reported to have homophilic interaction with itself and heterophilic interaction with nectin-1 (Samanta and Almo, Nectin family of cell-adhesion molecules: structural and molecular aspects of function and specificity, Cell Mol. Life Sci. 2015 February; 72(4):645-58). The soluble form of nectin-4 is produced by proteolytic cleavage by metalloproteinases ADAM10/17 and can potentially function as a prognostic marker.

Nectin-4 is a tumor-associated protein with overexpression on esophageal cancer, stomach cancer, bladder cancer, liver cancer, pancreatic cancer, ovarian cancer, breast cancer, colon cancer, gall bladder cancer, and lung cancer. Aberrant expression on tumors has been associated with promoting proliferation and metastasis. Nectin-4 overexpression is also associated with poor prognosis in many tumor types. In normal human tissues, expression is originally restricted in the embryo and placenta but weak to medium levels of nectin-4 expression is detected in the skin, esophagus, breast, oral mucosa, urinary bladder, placenta and tonsil (protein atlas).

Pathogen-associated molecular patterns (PAMPs) are molecules associated with various pathogens and are recognized by toll-like receptors (TLRs) and other pattern recognition receptors (PRRs) activating innate immune responses. The ability of PAMPs to recruit immune system in the absence of pathogens provides a strategy for treating a variety of diseases involving cell destruction (e.g., anticancer therapy) through the use of innate immune system response. One class of PAMPs that has been investigated for a variety of therapeutic applications is immunostimulating oligonucleotides, such as unmethylated cytosine-guanine dinucleotide (CpG)-containing oligodeoxynucleotides (CpG ODNs) (e.g., agatolimod). It is thought that CpG ODNs mediate TLR9 dimerization in immune cells (e.g., B cells, monocytes and plasmacytoid dendritic cells (pDCs)) to upregulate cytokines (e.g., type I interferon and interleukins), thereby activating natural killer cells.

Toll-like receptor 9 (TLR9), also designated as CD289, is an important receptor expressed in immune system cells including dendritic cells (DCs), B lymphocytes, macrophages, natural killer cells, and other antigen presenting cells. TLR9 activation triggers intracellular signaling cascades, leading to activation, maturation, proliferation and cytokine productions in these immune cells, thus bridges the innate and adaptive immunity. Martinez-Campos et al., Viral Immunol. 2016, 30, 98-105; Notley et al., Sci. Rep. 2017, 7, 42204. Natural TLR-9 agonists include unmethylated cytosine-guanine dinucleotide (CpG)-containing oligodeoxynucleotides (CpG ODNs).

CpG ODNs may include, for example, oligodeoxynucleotides comprising poly-G tails with phosphorothioate backbones at 3′- and 5′-termini and a central palindromic sequence including a phosphate backbone and a CpG within its central palindrome sequence, or oligodeoxynucleotides having a fully phosphorothioate backbone, and a sequence at the 5′ end for TLR9 activation, or oligodeoxynucleotides having a fully phosphorothioate backbone with a 3′-end sequence enabling formation of a duplex. However, CpG ODNs are often susceptible to degradation in serum and thus pharmacokinetics of CpG ODNs may be one of the limiting factors in their development as therapeutics. Also CpG ODNs often exhibit uneven tissue distribution in vivo, with primary sites of accumulation being in liver, kidney, and spleen. Such distribution can elicit off-target activity and local toxicity associated with PAMPs. Therefore, it will be advantageous to design CpG with improved stability properties, potency, and delivery systems.

There is a need for anti-nectin-4 antibodies conjugated to immunomodulating oligonucleotides to treat diseases, disorders, and conditions, such as cancer.

### BRIEF SUMMARY

In one aspect, provided herein is a conjugate comprising (i) an anti-nectin-4 antibody or antigen binding fragment thereof and (ii) one or more immunomodulating oligonucleotides (P), wherein the antibody or antigen-binding fragment is linked to one or more Q-tag peptides (Q) comprising at least one glutamine residue, and wherein each immunomodulating oligonucleotide is linked to a Q-tag peptide via an amide bond with the glutamine residue of the Q-tag peptide and a linker (L) as shown in Formula (A):

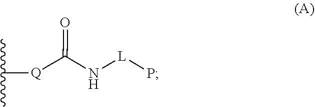

wherein [symbol] indicates the point of attachment of each Q to the antibody or antigen-binding fragment thereof (Ab);
wherein each P is independently an immunomodulating oligonucleotide comprising the structure

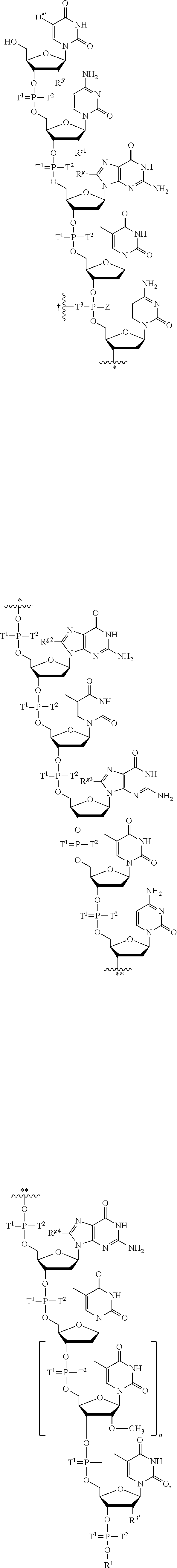

wherein
[symbol] * and [symbol] ** indicate the points of attachment within the oligonucleotide, and wherein [symbol] † indicates the point of attachment to the linker L;
each T¹ is independently O or S;
each T² is S⁻;
T³ is a group

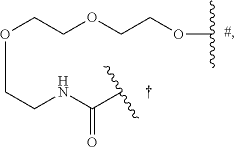 [SMILES: *OCCOCCOCCNC(*)=O]

wherein [symbol] † indicates the point of attachment to L and wherein [symbol] indicates the point of attachment to the rest of the oligonucleotide;
Z is O or S;
U^{5′} is —H or halogen;
R^{5′} is —H or methoxy;
R^{c1} is —H or methoxy;
R^{g1}, R^{g2}, R^{g3}, and R^{g4} are H;
R^{3′} is methoxy;
R¹ is —(CH₂)₃—OH;
R² is —H or methyl; and
n is an integer from 0 to 2.

In one aspect, provided herein is a conjugate comprising (i) an anti-nectin-4 antibody or antigen binding fragment thereof and (ii) and one or more immunomodulating oligonucleotides (P), wherein the antibody or antigen-binding fragment is linked to one or more Q-tag peptides (Q), and wherein each immunomodulating oligonucleotide is linked to a Q-tag peptide via an amide bond with the glutamine residue of the Q-tag peptide and a linker (L) as shown in Formula (A)

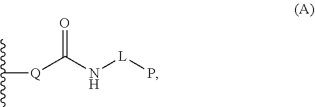

wherein:
[symbol] indicates the point of attachment of each Q to the antibody or antigen-binding fragment thereof (Ab)
each Q independently comprises a Q-tag peptide sequence RPQGF (SEQ ID NO:47);
each L is independently a bond or a linker moiety

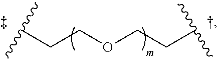

wherein m is an integer ranging from about 0 to about 50, and wherein [symbol] † indicates the point of attachment to P, and [symbol] ‡ indicates the point of attachment to the rest of the conjugate connected to Q via an amide bond with the glutamine residue; and
each P is independently an immunomodulating oligonucleotide having the structure

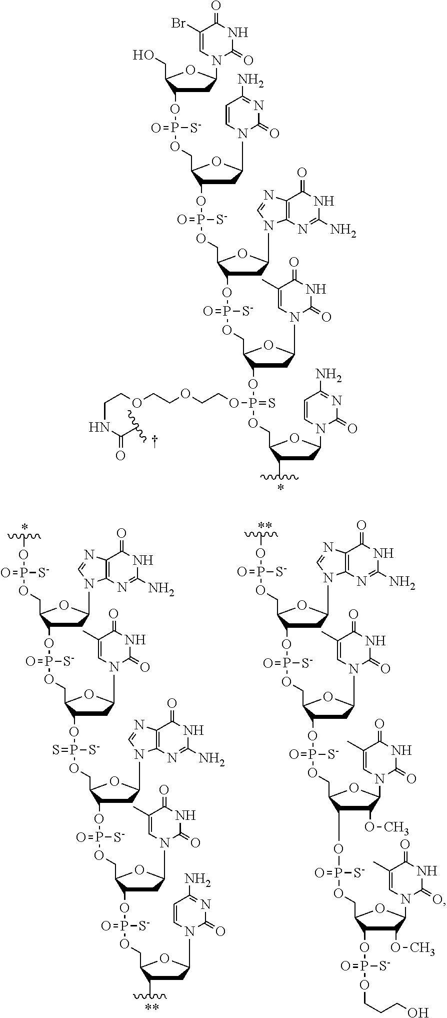

wherein [symbol] * and [symbol] ** indicate the points of attachment within the oligonucleotide, and
wherein [symbol] † indicates the point of attachment to the linker L.

In one aspect, provided herein is a conjugate comprising (i) an anti-nectin-4 antibody or antigen binding fragment thereof and (ii) one or more immunomodulating oligonucleotides (P), wherein the antibody or antigen-binding fragment is linked to one or more Q-tag peptides (Q), and wherein each immunomodulating oligonucleotide is linked to a Q-tag peptide via an amide bond with the glutamine residue of the Q-tag peptide and a linker (L) as shown in Formula (A)

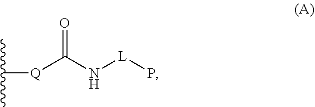

wherein:
[symbol] indicates the point of attachment of each Q to the antibody or antigen-binding fragment thereof (Ab)
each Q independently comprises a Q-tag peptide sequence RPQGF (SEQ ID NO:47);
each L is independently a bond or a linker moiety

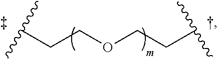

wherein m is an integer ranging from about 0 to about 50 (such as about 0 to about 10, about 0 to about 30, about 10 to about 30, about 20 to about 30, and values in ranges therebetween), and wherein [symbol] † indicates the point of attachment to P, and [symbol] ‡ indicates the point of attachment to the rest of the conjugate connected to Q via an amide bond with the glutamine residue; and
each P is independently an immunomodulating oligonucleotide comprising the structure

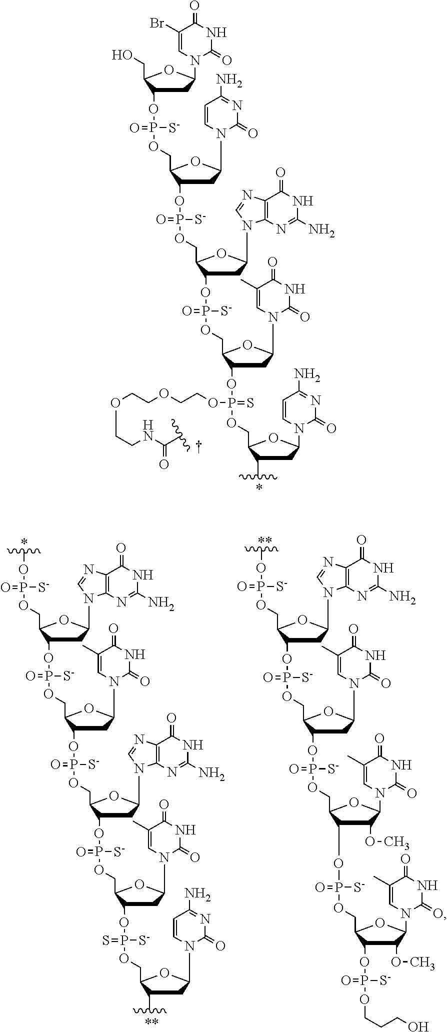

wherein [symbol] * and [symbol] ** indicate the points of attachment within the oligonucleotide, and
wherein [symbol] † indicates the point of attachment to the linker L.

In one aspect, provided herein is a conjugate comprising (i) an anti-nectin-4 antibody or antigen binding fragment thereof and (ii) one or more immunomodulating oligonucleotides (P),
wherein the antibody or antigen-binding fragment is linked to one or more Q-tag peptides (Q) that comprise the amino acid sequence RPQGF (SEQ ID NO:47),
and wherein each immunomodulating oligonucleotide (P) is linked to a Q-tag peptide via an amide bond with the glutamine residue of the Q-tag peptide and a linker (L) as shown in Formula (A):

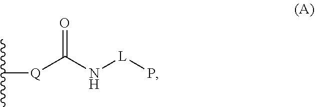

wherein [symbol] indicates the point of attachment of each Q to the antibody or antigen-binding fragment thereof (Ab).

In one aspect, provided herein is a conjugate comprising an anti-nectin-4 antibody or antigen binding fragment thereof, wherein the antibody comprises two antibody light chains, two antibody heavy chains, and two Q-tag peptides each comprising a peptide sequence RPQGF (SEQ ID NO:47); wherein each of the Q-tag peptides is linked to the C-terminus of one of the antibody heavy chains; and wherein at least one of the Q-tag peptides is linked to an immunomodulating oligonucleotide (P) via an amide bond with the glutamine residue of the Q-tag peptide and linker (L) as shown in FIG. 9A or FIG. 9B.

In one aspect, provided herein is a conjugate comprising an anti-nectin-4 antibody or antigen binding fragment thereof (Ab), at least one Q-tag peptide sequence comprising a glutamine residue, and at least one immunomodulatory oligonucleotide (P), wherein the Q-tag peptide sequence is naturally occurring or synthetic, and wherein each immunomodulatory oligonucleotide is linked to a Q-tag via an amide bond with the glutamine residue and linker (L), wherein at least one Q-tag peptide sequence is selected from the group consisting of SEQ ID NOs:39-55.

In some embodiments, the antibody of the conjugate comprises a light chain variable region (VL) domain and a heavy chain variable region (VH) domain, wherein:
(a) the VL domain comprises CDR-L1, CDR-L2, and CDR-L3 sequences from a VL domain selected from the group consisting of SEQ ID NOs:248, 250, 252, 254, 256, 258, 260, 262, 264, 266, 268, 270, 900, 902, 904, 906, 908, 910, 912, 914, 916, 918, 920, 922, 924, 926, 928, 930, 932, 934, 936, 938, 940, 942, and 944 and the VH domain comprises CDR-H1, CDR-H2, and CDR-H3 sequences from a VH domain selected from the group consisting of SEQ ID NOs:249, 251, 253, 255, 257, 259, 261, 263, 265, 267, 269, 271, 901, 903, 905, 907, 909, 911, 913, 915, 917, 919, 921, 923, 925, 927, 929, 931, 933, 935, 937, 939, 941, 943, and 945;
(b) the VL domain comprises CDR-L1, CDR-L2, and CDR-L3 sequences from a VL domain of SEQ ID NO:248 and the VH domain comprises CDR-H1, CDR-H2, and CDR-H3 from a VH domain of SEQ ID NO:249;
(c) the VL domain comprises CDR-L1, CDR-L2, and CDR-L3 sequences from a VL domain of SEQ ID NO:250 and the VH domain comprises CDR-H1, CDR-H2, and CDR-H3 from a VH domain of SEQ ID NO:251;
(d) the VL domain comprises CDR-L1, CDR-L2, and CDR-L3 sequences from a VL domain of SEQ ID NO:252 and the VH domain comprises CDR-H1, CDR-H2, and CDR-H3 from a VH domain of SEQ ID NO:253;
(e) the VL domain comprises CDR-L1, CDR-L2, and CDR-L3 sequences from a VL domain of SEQ ID NO:254 and the VH domain comprises CDR-H1, CDR-H2, and CDR-H3 from a VH domain of SEQ ID NO:255;
(f) the VL domain comprises CDR-L1, CDR-L2, and CDR-L3 sequences from a VL domain of SEQ ID NO:256 and the VH domain comprises CDR-H1, CDR-H2, and CDR-H3 from a VH domain of SEQ ID NO:257;
(g) the VL domain comprises CDR-L1, CDR-L2, and CDR-L3 sequences from a VL domain of SEQ ID NO:258 and the VH domain comprises CDR-H1, CDR-H2, and CDR-H3 from a VH domain of SEQ ID NO:259;
(h) the VL domain comprises CDR-L1, CDR-L2, and CDR-L3 sequences from a VL domain of SEQ ID NO:260 and the VH domain comprises CDR-H1, CDR-H2, and CDR-H3 from a VH domain of SEQ ID NO:261;
(i) the VL domain comprises CDR-L1, CDR-L2, and CDR-L3 sequences from a VL domain of SEQ ID NO:262 and the VH domain comprises CDR-H1, CDR-H2, and CDR-H3 from a VH domain of SEQ ID NO:263;
(j) the VL domain comprises CDR-L1, CDR-L2, and CDR-L3 sequences from a VL domain of SEQ ID NO:264 and the VH domain comprises CDR-H1, CDR-H2, and CDR-H3 from a VH domain of SEQ ID NO:265;
(k) the VL domain comprises CDR-L1, CDR-L2, and CDR-L3 sequences from a VL domain of SEQ ID NO:266 and the VH domain comprises CDR-H1, CDR-H2, and CDR-H3 from a VH domain of SEQ ID NO:267;
(1) the VL domain comprises CDR-L1, CDR-L2, and CDR-L3 sequences from a VL domain of SEQ ID NO:268 and the VH domain comprises CDR-H1, CDR-H2, and CDR-H3 from a VH domain of SEQ ID NO:269;
(m) the VL domain comprises CDR-L1, CDR-L2, and CDR-L3 sequences from a VL domain of SEQ ID NO:270 and the VH domain comprises CDR-H1, CDR-H2, and CDR-H3 from a VH domain of SEQ ID NO:271;
(n) the VL domain comprises CDR-L1, CDR-L2, and CDR-L3 sequences from a VL domain of SEQ ID NO:900 and the VH domain comprises CDR-H1, CDR-H2, and CDR-H3 from a VH domain of SEQ ID NO:901;
(o) the VL domain comprises CDR-L1, CDR-L2, and CDR-L3 sequences from a VL domain of SEQ ID NO:902 and the VH domain comprises CDR-H1, CDR-H2, and CDR-H3 from a VH domain of SEQ ID NO:903;
(p) the VL domain comprises CDR-L1, CDR-L2, and CDR-L3 sequences from a VL domain of SEQ ID NO:904 and the VH domain comprises CDR-H1, CDR-H2, and CDR-H3 from a VH domain of SEQ ID NO:905;
(q) the VL domain comprises CDR-L1, CDR-L2, and CDR-L3 sequences from a VL domain of SEQ ID NO:906 and the VH domain comprises CDR-H1, CDR-H2, and CDR-H3 from a VH domain of SEQ ID NO:907;
(r) the VL domain comprises CDR-L1, CDR-L2, and CDR-L3 sequences from a VL domain of SEQ ID NO:908 and the VH domain comprises CDR-H1, CDR-H2, and CDR-H3 from a VH domain of SEQ ID NO:909;
(s) the VL domain comprises CDR-L1, CDR-L2, and CDR-L3 sequences from a VL domain of SEQ ID NO:910 and the VH domain comprises CDR-H1, CDR-H2, and CDR-H3 from a VH domain of SEQ ID NO:911;
(t) the VL domain comprises CDR-L1, CDR-L2, and CDR-L3 sequences from a VL domain of SEQ ID NO:912 and the VH domain comprises CDR-H1, CDR-H2, and CDR-H3 from a VH domain of SEQ ID NO:913;
(u) the VL domain comprises CDR-L1, CDR-L2, and CDR-L3 sequences from a VL domain of SEQ ID NO:914 and the VH domain comprises CDR-H1, CDR-H2, and CDR-H3 from a VH domain of SEQ ID NO:915;
(v) the VL domain comprises CDR-L1, CDR-L2, and CDR-L3 sequences from a VL domain of SEQ ID NO:916 and the VH domain comprises CDR-H1, CDR-H2, and CDR-H3 from a VH domain of SEQ ID NO:917;
(w) the VL domain comprises CDR-L1, CDR-L2, and CDR-L3 sequences from a VL domain of SEQ ID NO:918 and the VH domain comprises CDR-H1, CDR-H2, and CDR-H3 from a VH domain of SEQ ID NO:919;
(x) the VL domain comprises CDR-L1, CDR-L2, and CDR-L3 sequences from a VL domain of SEQ ID NO:920 and the VH domain comprises CDR-H1, CDR-H2, and CDR-H3 from a VH domain of SEQ ID NO:921;
(y) the VL domain comprises CDR-L1, CDR-L2, and CDR-L3 sequences from a VL domain of SEQ ID NO:922 and the VH domain comprises CDR-H1, CDR-H2, and CDR-H3 from a VH domain of SEQ ID NO:923;
(z) the VL domain comprises CDR-L1, CDR-L2, and CDR-L3 sequences from a VL domain of SEQ ID NO:924 and the VH domain comprises CDR-H1, CDR-H2, and CDR-H3 from a VH domain of SEQ ID NO:925;
(aa) the VL domain comprises CDR-L1, CDR-L2, and CDR-L3 sequences from a VL domain of SEQ ID NO:926 and the VH domain comprises CDR-H1, CDR-H2, and CDR-H3 from a VH domain of SEQ ID NO:927;
(bb) the VL domain comprises CDR-L1, CDR-L2, and CDR-L3 sequences from a VL domain of SEQ ID NO:928 and the VH domain comprises CDR-H1, CDR-H2, and CDR-H3 from a VH domain of SEQ ID NO:929;
(cc) the VL domain comprises CDR-L1, CDR-L2, and CDR-L3 sequences from a VL domain of SEQ ID NO:930 and the VH domain comprises CDR-H1, CDR-H2, and CDR-H3 from a VH domain of SEQ ID NO:931;
(dd) the VL domain comprises CDR-L1, CDR-L2, and CDR-L3 sequences from a VL domain of SEQ ID NO932; and the VH domain comprises CDR-H1, CDR-H2, and CDR-H3 from a VH domain of SEQ ID NO:933;
(ee) the VL domain comprises CDR-L1, CDR-L2, and CDR-L3 sequences from a VL domain of SEQ ID NO:934 and the VH domain comprises CDR-H1, CDR-H2, and CDR-H3 from a VH domain of SEQ ID NO:935;
(ff) the VL domain comprises CDR-L1, CDR-L2, and CDR-L3 sequences from a VL domain of SEQ ID NO:936 and the VH domain comprises CDR-H1, CDR-H2, and CDR-H3 from a VH domain of SEQ ID NO:937;
(gg) the VL domain comprises CDR-L1, CDR-L2, and CDR-L3 sequences from a VL domain of SEQ ID NO:938 and the VH domain comprises CDR-H1, CDR-H2, and CDR-H3 from a VH domain of SEQ ID NO:939;
(hh) the VL domain comprises CDR-L1, CDR-L2, and CDR-L3 sequences from a VL domain of SEQ ID NO:940 and the VH domain comprises CDR-H1, CDR-H2, and CDR-H3 from a VH domain of SEQ ID NO:941;
(ii) the VL domain comprises CDR-L1, CDR-L2, and CDR-L3 sequences from a VL domain of SEQ ID NO:942 and the VH domain comprises CDR-H1, CDR-H2, and CDR-H3 from a VH domain of SEQ ID NO:943; or
(jj) the VL domain comprises CDR-L1, CDR-L2, and CDR-L3 sequences from a VL domain of SEQ ID NO:944 and the VH domain comprises CDR-H1, CDR-H2, and CDR-H3 from a VH domain of SEQ ID NO:945.

In some embodiments, the antibody of the conjugate comprises a heavy chain variable region (VH) domain and a light chain variable region (VL) domain, wherein:
(a) the VH domain comprises CDR-H1, CDR-H2, and CDR-H3 and the VL domain comprises CDR-L1, CDR-L2, and CDR-L3, wherein CDR-H1 comprises SEQ ID NO:275, CDR-H2 comprises SEQ ID NO:276, CDR-H3 comprises SEQ ID NO:277, CDR-L1 comprises SEQ ID NO:272, CDR-L2 comprises SEQ ID NO:273, and CDR-L3 comprises SEQ ID NO:274;
(b) the VH domain comprises CDR-H1, CDR-H2, and CDR-H3 and the VL domain comprises CDR-L1, CDR-L2, and CDR-L3, wherein CDR-H1 comprises SEQ ID NO:293, CDR-H2 comprises SEQ ID NO:294, CDR-H3 comprises SEQ ID NO:295, CDR-L1 comprises SEQ ID NO:290, CDR-L2 comprises SEQ ID NO:291, and CDR-L3 comprises SEQ ID NO:292;
(c) the VH domain comprises CDR-H1, CDR-H2, and CDR-H3 and the VL domain comprises CDR-L1, CDR-L2, and CDR-L3, wherein CDR-H1 comprises SEQ ID NO:311, CDR-H2 comprises SEQ ID NO:312, CDR-H3 comprises SEQ ID NO:313, CDR-L1 comprises SEQ ID NO:308, CDR-L2 comprises SEQ ID NO:309, and CDR-L3 comprises SEQ ID NO:310;
(d) the VH domain comprises CDR-H1, CDR-H2, and CDR-H3 and the VL domain comprises CDR-L1, CDR-L2, and CDR-L3, wherein CDR-H1 comprises SEQ ID NO:329, CDR-H2 comprises SEQ ID NO:330, CDR-H3 comprises SEQ ID NO:331, CDR-L1 comprises SEQ ID NO:326, CDR-L2 comprises SEQ ID NO:327, and CDR-L3 comprises SEQ ID NO:328;
(e) the VH domain comprises CDR-H1, CDR-H2, and CDR-H3 and the VL domain comprises CDR-L1, CDR-L2, and CDR-L3, wherein CDR-H1 comprises SEQ ID NO:347, CDR-H2 comprises SEQ ID NO:348, CDR-H3 comprises SEQ ID NO:349, CDR-L1 comprises SEQ ID NO:344, CDR-L2 comprises SEQ ID NO:345, and CDR-L3 comprises SEQ ID NO:346;
(f) the VH domain comprises CDR-H1, CDR-H2, and CDR-H3 and the VL domain comprises CDR-L1, CDR-L2, and CDR-L3, wherein CDR-H1 comprises SEQ ID NO:365, CDR-H2 comprises SEQ ID NO:366, CDR-H3 comprises SEQ ID NO:367, CDR-L1 comprises SEQ ID NO:362, CDR-L2 comprises SEQ ID NO:363, and CDR-L3 comprises SEQ ID NO:364;
(g) the VH domain comprises CDR-H1, CDR-H2, and CDR-H3 and the VL domain comprises CDR-L1, CDR-L2, and CDR-L3, wherein CDR-H1 comprises SEQ ID NO:383, CDR-H2 comprises SEQ ID NO:384, CDR-H3 comprises SEQ ID NO:385, CDR-L1 comprises SEQ ID NO:380, CDR-L2 comprises SEQ ID NO:381, and CDR-L3 comprises SEQ ID NO:382;
(h) the VH domain comprises CDR-H1, CDR-H2, and CDR-H3 and the VL domain comprises CDR-L1, CDR-L2, and CDR-L3, wherein CDR-H1 comprises SEQ ID NO:401, CDR-H2 comprises SEQ ID NO:402, CDR-H3 comprises SEQ ID NO:403, CDR-L1 comprises SEQ ID NO:398, CDR-L2 comprises SEQ ID NO:399, and CDR-L3 comprises SEQ ID NO:400;
(i) the VH domain comprises CDR-H1, CDR-H2, and CDR-H3 and the VL domain comprises CDR-L1, CDR-L2, and CDR-L3, wherein CDR-H1 comprises SEQ ID NO:419, CDR-H2 comprises SEQ ID NO:420, CDR-H3 comprises SEQ ID NO:421, CDR-L1 comprises SEQ ID NO:416, CDR-L2 comprises SEQ ID NO:417, and CDR-L3 comprises SEQ ID NO:418;
(j) the VH domain comprises CDR-H1, CDR-H2, and CDR-H3 and the VL domain comprises CDR-L1, CDR-L2, and CDR-L3, wherein CDR-H1 comprises SEQ ID NO:437, CDR-H2 comprises SEQ ID NO:438, CDR-H3 comprises SEQ ID NO:439, CDR-L1 comprises SEQ ID NO:434, CDR-L2 comprises SEQ ID NO:435, and CDR-L3 comprises SEQ ID NO:436;
(k) the VH domain comprises CDR-H1, CDR-H2, and CDR-H3 and the VL domain comprises CDR-L1, CDR-L2, and CDR-L3, wherein CDR-H1 comprises SEQ ID NO:455, CDR-H2 comprises SEQ ID NO:456, CDR-H3 comprises SEQ ID NO:457, CDR-L1 comprises SEQ ID NO:452, CDR-L2 comprises SEQ ID NO:453, and CDR-L3 comprises SEQ ID NO:454;
(l) the VH domain comprises CDR-H1, CDR-H2, and CDR-H3 and the VL domain comprises CDR-L1, CDR-L2, and CDR-L3, wherein CDR-H1 comprises SEQ ID NO:281, CDR-H2 comprises SEQ ID NO:282, CDR-H3 comprises SEQ ID NO:283, CDR-L1 comprises SEQ ID NO:278, CDR-L2 comprises SEQ ID NO:279, and CDR-L3 comprises SEQ ID NO:280;
(m) the VH domain comprises CDR-H1, CDR-H2, and CDR-H3 and the VL domain comprises CDR-L1, CDR-L2, and CDR-L3, wherein CDR-H1 comprises SEQ ID NO:299, CDR-H2 comprises SEQ ID NO:300, CDR-H3 comprises SEQ ID NO:301, CDR-L1 comprises SEQ ID NO:296, CDR-L2 comprises SEQ ID NO:297, and CDR-L3 comprises SEQ ID NO:298;
(n) the VH domain comprises CDR-H1, CDR-H2, and CDR-H3 and the VL domain comprises CDR-L1, CDR-L2, and CDR-L3, wherein CDR-H1 comprises SEQ ID NO:317, CDR-H2 comprises SEQ ID NO:318, CDR-H3 comprises SEQ ID NO:319, CDR-L1 comprises SEQ ID NO:314, CDR-L2 comprises SEQ ID NO:315, and CDR-L3 comprises SEQ ID NO:316;
(o) the VH domain comprises CDR-H1, CDR-H2, and CDR-H3 and the VL domain comprises CDR-L1, CDR-L2, and CDR-L3, wherein CDR-H1 comprises SEQ ID NO:335, CDR-H2 comprises SEQ ID NO:336, CDR-H3 comprises SEQ ID NO:337, CDR-L1 comprises SEQ ID NO:332, CDR-L2 comprises SEQ ID NO:333, and CDR-L3 comprises SEQ ID NO:334;
(p) the VH domain comprises CDR-H1, CDR-H2, and CDR-H3 and the VL domain comprises CDR-L1, CDR-L2, and CDR-L3, wherein CDR-H1 comprises SEQ ID NO:353, CDR-H2 comprises SEQ ID NO:354, CDR-H3 comprises SEQ ID NO:355, CDR-L1 comprises SEQ ID NO:350, CDR-L2 comprises SEQ ID NO:351, and CDR-L3 comprises SEQ ID NO:352;
(q) the VH domain comprises CDR-H1, CDR-H2, and CDR-H3 and the VL domain comprises CDR-L1, CDR-L2, and CDR-L3, wherein CDR-H1 comprises SEQ ID NO:371, CDR-H2 comprises SEQ ID NO:372, CDR-H3 comprises SEQ ID NO:373, CDR-L1 comprises SEQ ID NO:368, CDR-L2 comprises SEQ ID NO:369, and CDR-L3 comprises SEQ ID NO:370;
(r) the VH domain comprises CDR-H1, CDR-H2, and CDR-H3 and the VL domain comprises CDR-L1, CDR-L2, and CDR-L3, wherein CDR-H1 comprises SEQ ID NO:389, CDR-H2 comprises SEQ ID NO:390, CDR-H3 comprises SEQ ID NO:391, CDR-L1 comprises SEQ ID NO:386, CDR-L2 comprises SEQ ID NO:387, and CDR-L3 comprises SEQ ID NO:388;
(s) the VH domain comprises CDR-H1, CDR-H2, and CDR-H3 and the VL domain comprises CDR-L1, CDR-L2, and CDR-L3, wherein CDR-H1 comprises SEQ ID NO:407, CDR-H2 comprises SEQ ID NO:408, CDR-H3 comprises SEQ ID NO:409, CDR-L1 comprises SEQ ID NO:404, CDR-L2 comprises SEQ ID NO:405, and CDR-L3 comprises SEQ ID NO:406;
(t) the VH domain comprises CDR-H1, CDR-H2, and CDR-H3 and the VL domain comprises CDR-L1, CDR-L2, and CDR-L3, wherein CDR-H1 comprises SEQ ID NO:425, CDR-H2 comprises SEQ ID NO:426, CDR-H3 comprises SEQ ID NO:427, CDR-L1 comprises SEQ ID NO:422, CDR-L2 comprises SEQ ID NO:423, and CDR-L3 comprises SEQ ID NO:424;
(u) the VH domain comprises CDR-H1, CDR-H2, and CDR-H3 and the VL domain comprises CDR-L1, CDR-L2, and CDR-L3, wherein CDR-H1 comprises SEQ ID NO:443, CDR-H2 comprises SEQ ID NO:444, CDR-H3 comprises SEQ ID NO:445, CDR-L1 comprises SEQ ID NO:440, CDR-L2 comprises SEQ ID NO:441, and CDR-L3 comprises SEQ ID NO:442;
(v) the VH domain comprises CDR-H1, CDR-H2, and CDR-H3 and the VL domain comprises CDR-L1, CDR-L2, and CDR-L3, wherein CDR-H1 comprises SEQ ID NO:461, CDR-H2 comprises SEQ ID NO:462, CDR-H3 comprises SEQ ID NO:463, CDR-L1 comprises SEQ ID NO:458, CDR-L2 comprises SEQ ID NO:459, and CDR-L3 comprises SEQ ID NO:460;
(w) the VH domain comprises CDR-H1, CDR-H2, and CDR-H3 and the VL domain comprises CDR-L1, CDR-L2, and CDR-L3, wherein CDR-H1 comprises SEQ ID NO:287, CDR-H2 comprises SEQ ID NO:288, CDR-H3 comprises SEQ ID NO:289, CDR-L1 comprises SEQ ID NO:284, CDR-L2 comprises SEQ ID NO:285, and CDR-L3 comprises SEQ ID NO:286;
(x) the VH domain comprises CDR-H1, CDR-H2, and CDR-H3 and the VL domain comprises CDR-L1, CDR-L2, and CDR-L3, wherein CDR-H1 comprises SEQ ID NO:305, CDR-H2 comprises SEQ ID NO:306, CDR-H3 comprises SEQ ID NO:307, CDR-L1 comprises SEQ ID NO:302, CDR-L2 comprises SEQ ID NO:303, and CDR-L3 comprises SEQ ID NO:304;
(y) the VH domain comprises CDR-H1, CDR-H2, and CDR-H3 and the VL domain comprises CDR-L1, CDR-L2, and CDR-L3, wherein CDR-H1 comprises SEQ ID NO:323, CDR-H2 comprises SEQ ID NO:324, CDR-H3 comprises SEQ ID NO:325, CDR-L1 comprises SEQ ID NO:320, CDR-L2 comprises SEQ ID NO:321, and CDR-L3 comprises SEQ ID NO:322;
(z) the VH domain comprises CDR-H1, CDR-H2, and CDR-H3 and the VL domain comprises CDR-L1, CDR-L2, and CDR-L3, wherein CDR-H1 comprises SEQ ID NO:341, CDR-H2 comprises SEQ ID NO:342, CDR-H3 comprises SEQ ID NO:343, CDR-L1 comprises SEQ ID NO:338, CDR-L2 comprises SEQ ID NO:339, and CDR-L3 comprises SEQ ID NO:340;
(aa) the VH domain comprises CDR-H1, CDR-H2, and CDR-H3 and the VL domain comprises CDR-L1, CDR-L2, and CDR-L3, wherein CDR-H1 comprises SEQ ID NO:359, CDR-H2 comprises SEQ ID NO:360, CDR-H3 comprises SEQ ID NO:361, CDR-L1 comprises SEQ ID NO:356, CDR-L2 comprises SEQ ID NO:357, and CDR-L3 comprises SEQ ID NO:358;
(bb) the VH domain comprises CDR-H1, CDR-H2, and CDR-H3 and the VL domain comprises CDR-L1, CDR-L2, and CDR-L3, wherein CDR-H1 comprises SEQ ID NO:377, CDR-H2 comprises SEQ ID NO:378, CDR-H3 comprises SEQ ID NO:379, CDR-L1 comprises SEQ ID NO:374, CDR-L2 comprises SEQ ID NO:375, and CDR-L3 comprises SEQ ID NO:376;
(cc) the VH domain comprises CDR-H1, CDR-H2, and CDR-H3 and the VL domain comprises CDR-L1, CDR-L2, and CDR-L3, wherein CDR-H1 comprises SEQ ID NO:395, CDR-H2 comprises SEQ ID NO:396, CDR-H3 comprises SEQ ID NO:397, CDR-L1 comprises SEQ ID NO:392, CDR-L2 comprises SEQ ID NO:393, and CDR-L3 comprises SEQ ID NO:394;
(dd) the VH domain comprises CDR-H1, CDR-H2, and CDR-H3 and the VL domain comprises CDR-L1, CDR-L2, and CDR-L3, wherein CDR-H1 comprises SEQ ID NO:413, CDR-H2 comprises SEQ ID NO:414, CDR-H3 comprises SEQ ID NO:415, CDR-L1 comprises SEQ ID NO:410, CDR-L2 comprises SEQ ID NO:411, and CDR-L3 comprises SEQ ID NO:412;
(ee) the VH domain comprises CDR-H1, CDR-H2, and CDR-H3 and the VL domain comprises CDR-L1, CDR-L2, and CDR-L3, wherein CDR-H1 comprises SEQ ID NO:431, CDR-H2 comprises SEQ ID NO:432, CDR-H3 comprises SEQ ID NO:433, CDR-L1 comprises SEQ ID NO:428, CDR-L2 comprises SEQ ID NO:429, and CDR-L3 comprises SEQ ID NO:430;
(ff) the VH domain comprises CDR-H1, CDR-H2, and CDR-H3 and the VL domain comprises CDR-L1, CDR-L2, and CDR-L3, wherein CDR-H1 comprises SEQ ID NO:449, CDR-H2 comprises SEQ ID NO:450, CDR-H3 comprises SEQ ID NO:451, CDR-L1 comprises SEQ ID NO:446, CDR-L2 comprises SEQ ID NO:447, and CDR-L3 comprises SEQ ID NO:448;
(gg) the VH domain comprises CDR-H1, CDR-H2, and CDR-H3 and the VL domain comprises CDR-L1, CDR-L2, and CDR-L3, wherein CDR-H1 comprises SEQ ID NO:467, CDR-H2 comprises SEQ ID NO:468, CDR-H3 comprises SEQ ID NO:469, CDR-L1 comprises SEQ ID NO:464, CDR-L2 comprises SEQ ID NO:465, and CDR-L3 comprises SEQ ID NO:466;
(hh) the VH domain comprises CDR-H1, CDR-H2, and CDR-H3 and the VL domain comprises CDR-L1, CDR-L2, and CDR-L3, wherein CDR-H1 comprises SEQ ID NO:479, CDR-H2 comprises SEQ ID NO:480, CDR-H3 comprises SEQ ID NO:481, CDR-L1 comprises SEQ ID NO:476, CDR-L2 comprises SEQ ID NO:477, and CDR-L3 comprises SEQ ID NO:478;
(ii) the VH domain comprises CDR-H1, CDR-H2, and CDR-H3 and the VL domain comprises CDR-L1, CDR-L2, and CDR-L3, wherein CDR-H1 comprises SEQ ID NO:485, CDR-H2 comprises SEQ ID NO:486, CDR-H3 comprises SEQ ID NO:487, CDR-L1 comprises SEQ ID NO:482, CDR-L2 comprises SEQ ID NO:483, and CDR-L3 comprises SEQ ID NO:484;
(jj) the VH domain comprises CDR-H1, CDR-H2, and CDR-H3 and the VL domain comprises CDR-L1, CDR-L2, and CDR-L3, wherein CDR-H1 comprises SEQ ID NO:473, CDR-H2 comprises SEQ ID NO:474, CDR-H3 comprises SEQ ID NO:475, CDR-L1 comprises SEQ ID NO:470, CDR-L2 comprises SEQ ID NO:471, and CDR-L3 comprises SEQ ID NO:472;
(kk) the VL domain comprises CDR-L1, CDR-L2, and CDR-L3 and the VH domain comprises CDR-H1, CDR-H2, and CDR-H3, wherein CDR-L1 comprises SEQ ID NO:562, CDR-L2 comprises SEQ ID NO:563, and CDR-L3 comprises SEQ ID NO:564, wherein CDR-H1 comprises SEQ ID NO:565, CDR-H2 comprises SEQ ID NO:566, and CDR-H3 comprises SEQ ID NO:567;
(ll) the VL domain comprises CDR-L1, CDR-L2, and CDR-L3 and the VH domain comprises CDR-H1, CDR-H2, and CDR-H3, wherein CDR-L1 comprises SEQ ID NO:580, CDR-L2 comprises SEQ ID NO:581, and CDR-L3 comprises SEQ ID NO:582, wherein CDR-H1 comprises SEQ ID NO:583, CDR-H2 comprises SEQ ID NO:584, and CDR-H3 comprises SEQ ID NO:585;
(mm) the VL domain comprises CDR-L1, CDR-L2, and CDR-L3 and the VH domain comprises CDR-H1, CDR-H2, and CDR-H3, wherein CDR-L1 comprises SEQ ID NO:598, CDR-L2 comprises SEQ ID NO:599, and CDR-L3 comprises SEQ ID NO:600, wherein CDR-H1 comprises SEQ ID NO:601, CDR-H2 comprises SEQ ID NO:602, and CDR-H3 comprises SEQ ID NO:603;
(nn) the VL domain comprises CDR-L1, CDR-L2, and CDR-L3 and the VH domain comprises CDR-H1, CDR-H2, and CDR-H3, wherein CDR-L1 comprises SEQ ID NO:616, CDR-L2 comprises SEQ ID NO:617, and CDR-L3 comprises SEQ ID NO:618, wherein CDR-H1 comprises SEQ ID NO:619, CDR-H2 comprises SEQ ID NO:620, and CDR-H3 comprises SEQ ID NO:621;
(oo) the VL domain comprises CDR-L1, CDR-L2, and CDR-L3 and the VH domain comprises CDR-H1, CDR-H2, and CDR-H3, wherein CDR-L1 comprises SEQ ID NO:634, CDR-L2 comprises SEQ ID NO:635, and CDR-L3 comprises SEQ ID NO:636, wherein CDR-H1 comprises SEQ ID NO:637, CDR-H2 comprises SEQ ID NO:638, and CDR-H3 comprises SEQ ID NO:639;
(pp) the VL domain comprises CDR-L1, CDR-L2, and CDR-L3 and the VH domain comprises CDR-H1, CDR-H2, and CDR-H3, wherein CDR-L1 comprises SEQ ID NO:652, CDR-L2 comprises SEQ ID NO:653, and CDR-L3 comprises SEQ ID NO:654, wherein CDR-H1 comprises SEQ ID NO:655, CDR-H2 comprises SEQ ID NO:656, and CDR-H3 comprises SEQ ID NO:657;
(qq) the VL domain comprises CDR-L1, CDR-L2, and CDR-L3 and the VH domain comprises CDR-H1, CDR-H2, and CDR-H3, wherein CDR-L1 comprises SEQ ID NO:670, CDR-L2 comprises SEQ ID NO:671, and CDR-L3 comprises SEQ ID NO:672, wherein CDR-H1 comprises SEQ ID NO:673, CDR-H2 comprises SEQ ID NO:674, and CDR-H3 comprises SEQ ID NO:675;
(rr) the VL domain comprises CDR-L1, CDR-L2, and CDR-L3 and the VH domain comprises CDR-H1, CDR-H2, and CDR-H3, wherein CDR-L1 comprises SEQ ID NO:688, CDR-L2 comprises SEQ ID NO:689, and CDR-L3 comprises SEQ ID NO:690, wherein CDR-H1 comprises SEQ ID NO:691, CDR-H2 comprises SEQ ID NO:692, and CDR-H3 comprises SEQ ID NO:693;
(ss) the VL domain comprises CDR-L1, CDR-L2, and CDR-L3 and the VH domain comprises CDR-H1, CDR-H2, and CDR-H3, wherein CDR-L1 comprises SEQ ID NO:706, CDR-L2 comprises SEQ ID NO:707, and CDR-L3 comprises SEQ ID NO:708, wherein CDR-H1 comprises SEQ ID NO:709, CDR-H2 comprises SEQ ID NO:710, and CDR-H3 comprises SEQ ID NO:711;
(tt) the VL domain comprises CDR-L1, CDR-L2, and CDR-L3 and the VH domain comprises CDR-H1, CDR-H2, and CDR-H3, wherein CDR-L1 comprises SEQ ID NO:724, CDR-L2 comprises SEQ ID NO:725, and CDR-L3 comprises SEQ ID NO:726, wherein CDR-H1 comprises SEQ ID NO:727, CDR-H2 comprises SEQ ID NO:728, and CDR-H3 comprises SEQ ID NO:729;
(uu) the VL domain comprises CDR-L1, CDR-L2, and CDR-L3 and the VH domain comprises CDR-H1, CDR-H2, and CDR-H3, wherein CDR-L1 comprises SEQ ID NO:742, CDR-L2 comprises SEQ ID NO:743, and CDR-L3 comprises SEQ ID NO:744, wherein CDR-H1 comprises SEQ ID NO:745, CDR-H2 comprises SEQ ID NO:746, and CDR-H3 comprises SEQ ID NO:747;
(vv) the VL domain comprises CDR-L1, CDR-L2, and CDR-L3 and the VH domain comprises CDR-H1, CDR-H2, and CDR-H3, wherein CDR-L1 comprises SEQ ID NO:760, CDR-L2 comprises SEQ ID NO:761, and CDR-L3 comprises SEQ ID NO:762, wherein CDR-H1 comprises SEQ ID NO:763, CDR-H2 comprises SEQ ID NO:764, and CDR-H3 comprises SEQ ID NO:765;
(ww) the VL domain comprises CDR-L1, CDR-L2, and CDR-L3 and the VH domain comprises CDR-H1, CDR-H2, and CDR-H3, wherein CDR-L1 comprises SEQ ID NO:778, CDR-L2 comprises SEQ ID NO:779, and CDR-L3 comprises SEQ ID NO:780, wherein CDR-H1 comprises SEQ ID NO:781, CDR-H2 comprises SEQ ID NO:782, and CDR-H3 comprises SEQ ID NO:783;
(xx) the VL domain comprises CDR-L1, CDR-L2, and CDR-L3 and the VH domain comprises CDR-H1, CDR-H2, and CDR-H3, wherein CDR-L1 comprises SEQ ID NO:796, CDR-L2 comprises SEQ ID NO:797, and CDR-L3 comprises SEQ ID NO:798, wherein CDR-H1 comprises SEQ ID NO:799, CDR-H2 comprises SEQ ID NO:800, and CDR-H3 comprises SEQ ID NO:801;
(yy) the VL domain comprises CDR-L1, CDR-L2, and CDR-L3 and the VH domain comprises CDR-H1, CDR-H2, and CDR-H3, wherein CDR-L1 comprises SEQ ID NO:814, CDR-L2 comprises SEQ ID NO:815, and CDR-L3 comprises SEQ ID NO:816, wherein CDR-H1 comprises SEQ ID NO:817, CDR-H2 comprises SEQ ID NO:818, and CDR-H3 comprises SEQ ID NO:819; or
(zz) the VL domain comprises CDR-L1, CDR-L2, and CDR-L3 and the VH domain comprises CDR-H1, CDR-H2, and CDR-H3, wherein CDR-L1 comprises SEQ ID NO:832, CDR-L2 comprises SEQ ID NO:833, and CDR-L3 comprises SEQ ID NO:834, wherein CDR-H1 comprises SEQ ID NO:835, CDR-H2 comprises SEQ ID NO:836, and CDR-H3 comprises SEQ ID NO:837.

In some embodiments, the antibody of the conjugate comprises a light chain variable region (VL) domain and a heavy chain variable region (VH) domain, wherein:
(a) the VH domain comprises the sequence of amino acids selected from the group consisting of SEQ ID NOs:249, 251, 253, 255, 257, 259, 261, 263, 265, 267, 269, 271, 901, 903, 905, 907, 909, 911, 913, 915, 917, 919, 921, 923, 925, 927, 929, 931, 933, 935, 937, 939, 941, 943, and 945 and/or the VL domain comprises the sequence of amino acids selected from the group consisting of SEQ ID NOs: 248, 250, 252, 254, 256, 258, 260, 262, 264, 266, 268, 270, 900, 902, 904, 906, 908, 910, 912, 914, 916, 918, 920, 922, 924, 926, 928, 930, 932, 934, 936, 938, 940, 942, and 944;
(b) the VL domain comprises the sequence of SEQ ID NO:248 and/or the VH domain comprises the sequence of SEQ ID NO:249;
(c) the VL domain comprises the sequence of SEQ ID NO:250 and/or the VH domain comprises the sequence of SEQ ID NO:251;
(d) the VL domain comprises the sequence of SEQ ID NO:252 and/or the VH domain comprises the sequence of SEQ ID NO:253;
(e) the VL domain comprises the sequence of SEQ ID NO:254 and/or the VH domain comprises the sequence of SEQ ID NO:255;
(f) the VL domain comprises the sequence of SEQ ID NO:256 and/or the VH domain comprises the sequence of SEQ ID NO:257;
(g) the VL domain comprises the sequence of SEQ ID NO:258 and/or the VH domain comprises the sequence of SEQ ID NO:259;
(h) the VL domain comprises the sequence of SEQ ID NO:260 and/or the VH domain comprises the sequence of SEQ ID NO:261;
(i) the VL domain comprises the sequence of SEQ ID NO:262 and/or the VH domain comprises the sequence of SEQ ID NO:263;
(j) the VL domain comprises the sequence of SEQ ID NO:264 and/or the VH domain comprises the sequence of SEQ ID NO:265;
(k) the VL domain comprises the sequence of SEQ ID NO:266 and/or the VH domain comprises the sequence of SEQ ID NO:267;
(1) the VL domain comprises the sequence of SEQ ID NO:268 and/or the VH domain comprises the sequence of SEQ ID NO:269;
(m) the VL domain comprises the sequence of SEQ ID NO:270 and/or the VH domain comprises the sequence of SEQ ID NO:271;
(n) the VL domain comprises the sequence of SEQ ID NO:900 and/or the VH domain comprises the sequence of SEQ ID NO:901;
(o) the VL domain comprises the sequence of SEQ ID NO:902 and/or the VH domain comprises the sequence of SEQ ID NO:903;
(p) the VL domain comprises the sequence of SEQ ID NO:904 and/or the VH domain comprises the sequence of SEQ ID NO:905;
(q) the VL domain comprises the sequence of SEQ ID NO:906 and/or the VH domain comprises the sequence of SEQ ID NO:907;
(r) the VL domain comprises the sequence of SEQ ID NO:908 and/or the VH domain comprises the sequence of SEQ ID NO:909;
(s) the VL domain comprises the sequence of SEQ ID NO:910 and/or the VH domain comprises the sequence of SEQ ID NO:911;
(t) the VL domain comprises the sequence of SEQ ID NO:912 and/or the VH domain comprises the sequence of SEQ ID NO:913;
(u) the VL domain comprises the sequence of SEQ ID NO:914 and/or the VH domain comprises the sequence of SEQ ID NO:915;
(v) the VL domain comprises the sequence of SEQ ID NO:916 and/or the VH domain comprises the sequence of SEQ ID NO:917;
(w) the VL domain comprises the sequence of SEQ ID NO:918 and/or the VH domain comprises the sequence of SEQ ID NO:919;
(x) the VL domain comprises the sequence of SEQ ID NO:920 and/or the VH domain comprises the sequence of SEQ ID NO:921;
(y) the VL domain comprises the sequence of SEQ ID NO:922 and/or the VH domain comprises the sequence of SEQ ID NO:923;
(z) the VL domain comprises the sequence of SEQ ID NO:924 and/or the VH domain comprises the sequence of SEQ ID NO:925;
(aa) the VL domain comprises the sequence of SEQ ID NO:926 and/or the VH domain comprises the sequence of SEQ ID NO:927;
(bb) the VL domain comprises the sequence of SEQ ID NO:928 and/or the VH domain comprises the sequence of SEQ ID NO:929;
(cc) the VL domain comprises the sequence of SEQ ID NO:930 and/or the VH domain comprises the sequence of SEQ ID NO:931;
(dd) the VL domain comprises the sequence of SEQ ID NO:932 and/or the VH domain comprises the sequence of SEQ ID NO:933;
(ee) the VL domain comprises the sequence of SEQ ID NO:934 and/or the VH domain comprises the sequence of SEQ ID NO:935;
(ff) the VL domain comprises the sequence of SEQ ID NO:936 and/or the VH domain comprises the sequence of SEQ ID NO:937;
(gg) the VL domain comprises the sequence of SEQ ID NO:938 and/or the VH domain comprises the sequence of SEQ ID NO:939;
(hh) the VL domain comprises the sequence of SEQ ID NO:940 and/or the VH domain comprises the sequence of SEQ ID NO:941;
(ii) the VL domain comprises the sequence of SEQ ID NO:942 and/or the VH domain comprises the sequence of SEQ ID NO:943; or
(jj) the VL domain comprises the sequence of SEQ ID NO:944 and/or the VH domain comprises the sequence of SEQ ID NO:945.

In some embodiments, the antibody of the conjugate is a monoclonal antibody. In some embodiments, the antibody of the conjugate is a Fab, F(ab′)2, Fab′-SH, Fv, scFv, single domain, single heavy chain, or single light chain antibody or antibody fragment. In some embodiments, the antibody of the conjugate is a humanized, human, or chimeric antibody or fragment thereof.

In some embodiments, the antibody of the conjugate comprises an Fc region. In some embodiments, the Fc region is a human Fc region selected from the group consisting of an IgG1 Fc region, an IgG2 Fc region, and an IgG4 Fc region. In some embodiments, the Fc region is a human Fc region comprising one or more amino acid substitutions that reduce binding to C1q. In some embodiments, the Fc region is a human Fc region comprising one or more amino acid substitutions that increase binding to C1q. In some embodiments, the Fc region is a human Fc region exhibiting wild-type complement activation. In some embodiments, the Fc region is a human Fc region comprising one or more amino acid substitutions that increase complement activation. In some embodiments, the Fc region is a human Fc region comprising one or more amino acid substitutions that reduce effector function, as compared with a human Fc region that lacks the amino acid substitution(s). In some embodiments, the Fc region is: (a) a human IgG1 Fc region comprising L234A, L235A, and/or G237A substitutions, amino acid position numbering according to EU index; (b) a human IgG2 Fc region comprising A330S and/or P331S substitutions, amino acid position numbering according to EU index; or (c) a human IgG4 Fc region comprising S228P and/or L235E substitutions, amino acid position numbering according to EU index. In some embodiments, the Fc region is a wild-type human IgG1 Fc region. In some embodiments, the Fc region is a human IgG1 Fc region. In some embodiments, the Fc region has been engineered to improve effector function, optionally wherein the improved effector function is antibody-dependent cell-mediated cytotoxicity (ADCC) activity, antibody-dependent cellular phagocytosis (ADCP) activity, or complement-dependent cytotoxicity (CDC) activity. In some embodiments, the Fc region has been engineered to exhibit at least two features selected from the group consisting of improved ADCC activity, improved ADCP activity, and improved CDC activity. In some embodiments, the Fc region comprises the substitution G236A, with amino acid position according to EU index. In some embodiments, the Fc region comprises the substitution G236A, with amino acid position according to EU index, and the Fc region is non-fucosylated. In some embodiments, the Fc region has been engineered to improve ADCC activity. In some embodiments, the Fc region has been engineered to improve ADCP activity. In some embodiments, the Fc region has been engineered to improve CDC activity. In some embodiments, the antibody of the conjugate comprises at least one amino acid substitution in the Fc region that improves ADCC activity. In some embodiments, the antibody of the conjugate comprises at least one amino acid substitution in the Fc region that improves ADCP activity. In some embodiments, the antibody of the conjugate comprises at least one amino acid substitution in the Fc region that improves CDC activity. In some embodiments, at least one or at least two of the heavy chains of the antibody of the conjugate is/are non-fucosylated. In some embodiments, the antibody of the conjugate is produced in a cell line having an alpha1,6-fucosyltransferase (Fut8) knockout or in a cell line overexpressing γ1,4-N-acetylglycosminyltransferase III (GnT-III), wherein optionally the cell line is a CHO cell line. In some embodiments, the antibody of the conjugate is produced in an expression system in the presence of a 2-fluorofucose compound or a 5-alkynylfucose derivative. In some embodiments, the cell line overexpresses Golgi μ-mannosidase II (ManII), wherein optionally the cell line is a CHO cell line. In some embodiments, the Fc region comprises an N297A substitution, amino acid position numbering according to EU index. In some embodiments, the Fc region comprises a D265A substitution, with amino acid position numbering according to EU index.

In some embodiments, the conjugate further comprises an immunomodulating oligonucleotide P attached to the Q295 of the Fc region residue as shown in the following formula

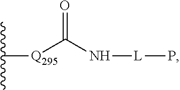

wherein L is a linker moiety connected to Q295 of the Fc region via an amide bond.

In some embodiments, the antibody of the conjugate comprises an antibody heavy chain constant domain comprising an amino acid sequence selected from the group consisting of SEQ ID NOs:92-107, 111,112, 178, and 494-497. In some embodiments, the antibody of the conjugate comprises a human lambda light chain. In some embodiments, the antibody of the conjugate comprises a human kappa light chain. In some embodiments, the antibody of the conjugate comprises an antibody light chain constant domain comprising an amino acid sequence selected from the group consisting of SEQ ID Nos:108-110. In some embodiments, the antibody of the conjugate comprises two antibody heavy chains and two antibody light chains, and wherein one Q-tag is attached to one or both heavy chain(s) of the antibody. In some embodiments, the Q-tag is fused to the C-terminus of the heavy chain of the antibody(ies). In some embodiments, the antibody of the conjugate comprises two antibody heavy chains and two antibody light chains, and wherein one Q-tag is attached to one or both light chain(s) of the antibody. In some embodiments, the Q-tag is within the Fc domain.

In some embodiments, the conjugate induces activation of TLR9. In some embodiments, each Q-tag independently comprises a peptide sequence comprising between 5 and 15 amino acid residues. In some embodiments, the Q-tag is naturally occurring. In some embodiments, the peptide sequence of each Q-tag is independently selected from the group consisting of SEQ ID NOs: 39-55. In some embodiments, the Q-tag comprises the peptide sequence RPQGF (SEQ ID NO:47). In some embodiments, each Q-tag independently comprises RPQGF (SEQ ID NO:47), RPQGFPP (SEQ ID NO:48), or RPQGFGPP (SEQ ID NO:49). In some embodiments, each Q-tag independently comprises RPQGFGPP (SEQ ID NO:49). In some embodiments, 1 or 2 Q-tags is/are linked to the antibody. In some embodiments, the conjugate has a DAR of 1 or 2.

In some embodiments, the linker L comprises a polyethylene glycol moiety. In some embodiments, the linker L is

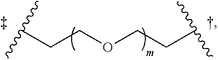

wherein m is an integer ranging from about 0 to about 50, and wherein [symbol] † indicates the point of attachment to T³, and [symbol] ‡ indicates the point of attachment to the rest of the conjugate

In some embodiments, m is about 20 to about 30. In some embodiments, wherein m is about 24. In some embodiments, Z is S. In some embodiments, the oligonucleotide P comprises at least one pair of geminal T¹ and T² wherein T¹ is S and T² is S⁻. In some embodiments, at least one pair of geminal T¹ and T² wherein T¹ is S and T² is S is located at the 3′-position of nucleoside residue 1. In some embodiments, at least one pair of geminal T¹ and T² wherein T¹ is S and T² is S is located at the 3′-position of nucleoside residue 2. In some embodiments, at least one pair of geminal T¹ and T² wherein T¹ is S and T² is S is located at the 3′-position of nucleoside residue 3. In some embodiments, at least one pair of geminal T¹ and T² wherein T¹ is S and T² is S is located at the 3′-position of nucleoside residue 5. In some embodiments, at least one pair of geminal T¹ and T² wherein T¹ is S and T² is S is located at the 3′-position of nucleoside residue 6. In some embodiments, at least one pair of geminal T¹ and T² wherein T¹ is S and T² is S is located at the 3′-position of nucleoside residue 7. In some embodiments, at least one pair of geminal T¹ and T² wherein T¹ is S and T² is S is located at the 3′-position of nucleoside residue 8. In some embodiments, at least one pair of geminal T¹ and T² wherein T¹ is S and T² is S is located at the 3′-position of nucleoside residue 9. In some embodiments, at least one pair of geminal T¹ and T² wherein T¹ is S and T² is S is located at the 3′-position of nucleoside residue 10. In some embodiments, at least one pair of geminal T¹ and T² wherein T¹ is S and T² is S is located at the 3′-position of nucleoside residue 11. In some embodiments, at least one pair of geminal T¹ and T² wherein T¹ is S and T² is S is located at the 3′-position of nucleoside residue 12. In some embodiments, at least one pair of geminal T¹ and T² wherein T¹ is S and T² is S is located at the 3′-position of nucleoside residue 13. In some embodiments, at least one pair of geminal T¹ and T² wherein T¹ is S and T² is S is located at the 3′-position of nucleoside residue 14. In some embodiments, at least one pair of geminal T¹ and T² wherein T¹ is S and T² is S is located at the 3′-position of nucleoside residue 15. In some embodiments, the oligonucleotide P comprises at least two pairs of geminal T¹ and T² wherein T¹ is S and T² is S⁻. In some embodiments, wherein R^{5′} is H. In some embodiments, R^{5′} is methoxy. In some embodiments, R^{c1} is H. In some embodiments, R^{c1} is methoxy. In some embodiments, R² is methyl. In some embodiments, R² is H. In some embodiments, U^{5′} is bromo. In some embodiments, U^{5′} is —H. In some embodiments, m is an integer from 20 to 25. In some embodiments, m is 24.

In some embodiments, each P independently comprises an oligonucleotide selected from Table 2, Table 3, and Table 4. In some embodiments, each (L) and (P) in the conjugate independently comprises a structure selected from Table 2, Table 5, and Table 6, optionally compound 7.6b or compound 7.7b. In some embodiments, each (P) and (L) in the conjugate independently comprises an oligonucleotide or a modified oligonucleotide selected from SEQ ID NOS: 1-38 and 129-166. In some embodiments, each immunomodulating oligonucleotide P is independently

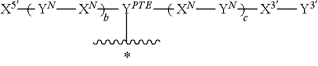

wherein
b and c are each independently an integer from 1 to 25; with the proviso that the sum of b and c is at least 5;
[symbol] * indicates the point of attachment of the immunomodulating oligonucleotide P to the rest of the conjugate;
X^{5′} is a 5′ terminal nucleoside comprising the structure

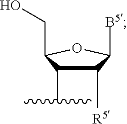

X^{3′} is a 3′ terminal nucleoside comprising the structure

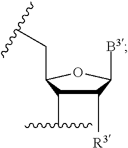

Y^{PTE} is an internucleoside phosphotriester comprising the structure

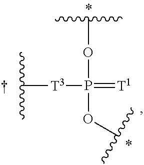

wherein * indicates the points of attachment to the rest of the oligonucleotide and [symbol] † indicates the point of attachment to the linker L, or, if L is absent, [symbol] † indicates the point of attachment to the Q tag peptide at the glutamine residue via an amide bond;
Y^{3′} is a terminal phosphotriester comprising the structure

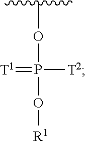

each X^{N} is independently a nucleoside comprising the structure

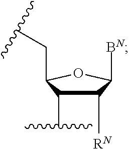

each Y^{N} is independently an internucleoside linker comprising the structure

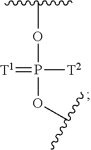

wherein each B^{N} is independently a modified or unmodified nucleobase;
each R^{N} is independently —H or —O—C₁₋₄-alkyl, wherein the C₁₋₄-alkyl of the —O—C₁₋₄-alkyl is optionally further substituted by —O—C₁-C₄-alkyl;
B^{5′} and B^{3′} are independently a modified or unmodified nucleobase;
R^{5′} and R^{3′} are independently —H or —O—C₁-C₄-alkyl, wherein the C₁₋₄-alkyl of the —O—C₁₋₄-alkyl is optionally further substituted by —O—C₁₋₄-alkyl;
each T₁ is independently O or S;
each T₂ is independently O⁻or S⁻; and
T₃ is a group comprising an oligoethylene glycol moiety; and R¹ is C₁₋₄-alkylene-hydroxy.

In some embodiments, b is 3. In some embodiments, (i) P comprises at least one modified nucleoside X^{N}; (ii) P has at least one modified internucleoside linker Y^{N}, wherein at least one of T¹ or T² is S; or (iii) both (i) and (ii). In some embodiments, P has at least one phosphorodithioate or phosphorothioate internucleoside linker. In some embodiments, P comprises 0, 1, 2, or 3 phosphorodithioate internucleoside linkers. In some embodiments, P comprises a modified nucleoside selected from the group consisting of 2′-O⁻alkyl nucleoside, 2′-O⁻alkoxyalkyl nucleoside, 2′-deoxynucleoside and ribonucleoside. In some embodiments, the modified nucleoside is selected from the group consisting of 5-bromo-2′-O⁻methyluridine, 5-bromo-2′-deoxyuridine, 2′-O⁻methyluridine, 2′-deoxyuridine, 2′-O⁻methylthymidine, 2′-O⁻methylcytidine, 2′-O⁻(2-methoxyethyl)thymidine and 8-oxo-7,8-dihydro-2′-deoxyguanosine. In some embodiments, X^{5′} is a 5-bromo-2′-O⁻methyluridine, 5-bromo-2′-deoxyuridine, 2′-O⁻methyluridine or 2′-deoxyuridine. In some embodiments, Y³ or the Y^{N} at the 3′ position of X^{5′} comprises an unsubstituted or substituted phosphorothioate. In some embodiments, Y^{PTE} is:

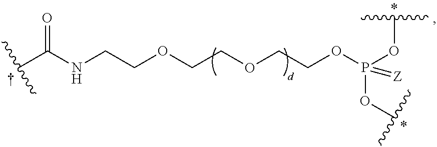

wherein Z is O or S; d is an integer from 0 to 95; the two [symbol] on the right side of the structure indicate the points of attachment to the adjacent nucleosides X^{N} in the oligonucleotide P, and the one [symbol] † the left side of the structure indicates the point of attachment to the linker L.

In some embodiments, Y^{PTE} is:

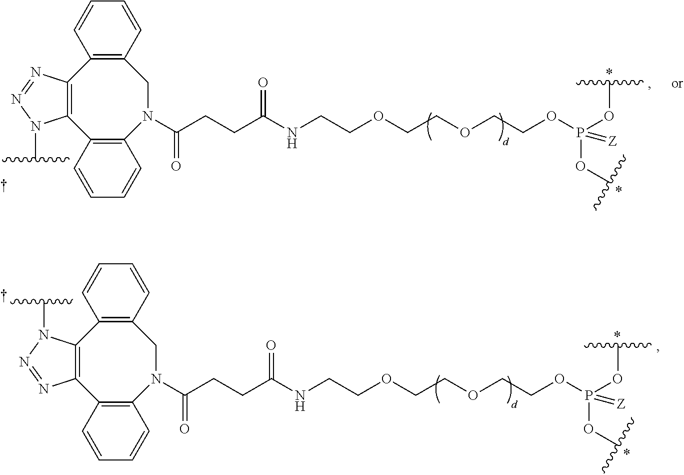

wherein Z is O or S; d is an integer from 0 to 95; the two [symbol] * on the right side of the structure indicate the points of attachment to the adjacent nucleosides X^{N} in the oligonucleotide P, and the one [symbol] † on the left side of the structure indicates the point of attachment to the linker L.

In some embodiments, Z is S. In some embodiments, d is an integer from 1 to 25. In some embodiments, the linker L comprises a polyethylene glycol moiety. In some embodiments, the linker L is

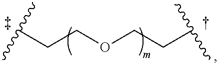

wherein m is an integer ranging from about 0 to about 50, and wherein t indicates the point of attachment to Y^{PTE}, and [symbol] ‡ indicates the point of attachment to the rest of the conjugate.

In some embodiments of the conjugate, P comprises one or more CpG sites. In some embodiments, P comprises at least 3 CpG sites. In some embodiments, the antibody of the conjugate comprises two antibody light chains, two antibody heavy chains, and two Q-tag peptides; wherein each of the Q-tag peptides is linked to the C-terminus of one of the antibody heavy chains; and wherein one of the Q-tag peptides is linked to an immunomodulating oligonucleotide (P) via an amide bond with the glutamine residue of the Q-tag peptide and linker (L).

In some embodiments of the conjugate:
(a) the antibody comprises a light chain comprising a light chain sequence selected from the group consisting of SEQ ID NOs:236-247 and 1000-1022 and/or a heavy chain comprising a heavy chain sequence and a Q-tag, wherein the heavy chain comprises a sequence selected from the group consisting of SEQ ID NOs:224-235 and 1030-1065;
(b) the antibody comprises a light chain comprising a light chain sequence selected from the group consisting of SEQ ID NOs:236-247 and 1000-1022 and/or a heavy chain comprising a heavy chain sequence selected from the group consisting of SEQ ID NOs:1070-1117;
(c) the antibody comprises a light chain comprising SEQ ID NO:236 and/or a heavy chain comprising SEQ ID NO:224;
(d) the antibody comprises a light chain comprising SEQ ID NO:237 and/or a heavy chain comprising SEQ ID NO:225;
(e) the antibody comprises a light chain comprising SEQ ID NO:238 and/or a heavy chain comprising SEQ ID NO:226;
(f) the antibody comprises a light chain comprising SEQ ID NO:239 and/or a heavy chain comprising SEQ ID NO:227;
(g) the antibody comprises a light chain comprising SEQ ID NO:240 and/or a heavy chain comprising SEQ ID NO:228;
(h) the antibody comprises a light chain comprising SEQ ID NO:241 and/or a heavy chain comprising SEQ ID NO:229;
(i) the antibody comprises a light chain comprising SEQ ID NO:242 and/or a heavy chain comprising SEQ ID NO:230;
(j) the antibody comprises a light chain comprising SEQ ID NO:243 and/or a heavy chain comprising SEQ ID NO:231;
(k) the antibody comprises a light chain comprising SEQ ID NO:244 and/or a heavy chain comprising SEQ ID NO:232;
(l) the antibody comprises a light chain comprising SEQ ID NO:245 and/or a heavy chain comprising SEQ ID NO:233;
(m) the antibody comprises a light chain comprising SEQ ID NO:246 and/or a heavy chain comprising SEQ ID NO:234;
(n) the antibody comprises a light chain comprising SEQ ID NO:247 and/or a heavy chain comprising SEQ ID NO:235;
(o) the antibody comprises a light chain comprising SEQ ID NO:1000 and/or a heavy chain comprising SEQ ID NO:1030;
(p) the antibody comprises a light chain comprising SEQ ID NO:1001 and/or a heavy chain comprising SEQ ID NO:1031;
(q) the antibody comprises a light chain comprising SEQ ID NO:1002 and/or a heavy chain comprising SEQ ID NO:1032;
(r) the antibody comprises a light chain comprising SEQ ID NO:1003 and/or a heavy chain comprising SEQ ID NO:1033;
(s) the antibody comprises a light chain comprising SEQ ID NO:1004 and/or a heavy chain comprising SEQ ID NO:1034;
(t) the antibody comprises a light chain comprising SEQ ID NO:1005 and/or a heavy chain comprising SEQ ID NO:1035;
(u) the antibody comprises a light chain comprising SEQ ID NO:1006 and/or a heavy chain comprising SEQ ID NO:1036;
(v) antibody comprises a light chain comprising SEQ ID NO:1007 and/or a heavy chain comprising SEQ ID NO:1037;
(w) the antibody comprises a light chain comprising SEQ ID NO:1008 and/or a heavy chain comprising SEQ ID NO:1038;
(x) the antibody comprises a light chain comprising SEQ ID NO:1009 and/or a heavy chain comprising SEQ ID NO:1039;
(y) the antibody comprises a light chain comprising SEQ ID NO:1010 and/or a heavy chain comprising SEQ ID NO:1040;
(z) the antibody comprises a light chain comprising SEQ ID NO:1011 and/or a heavy chain comprising SEQ ID NO:1041;
(aa) the antibody comprises a light chain comprising SEQ ID NO:1012 and/or a heavy chain comprising SEQ ID NO:1042;
(bb) the antibody comprises a light chain comprising SEQ ID NO:1013 and/or a heavy chain comprising SEQ ID NO:1043;
(cc) the antibody comprises a light chain comprising SEQ ID NO:1014 and/or a heavy chain comprising SEQ ID NO:1044;
(dd) the antibody comprises a light chain comprising SEQ ID NO:1015 and/or a heavy chain comprising SEQ ID NO:1045;
(ee) the antibody comprises a light chain comprising SEQ ID NO:1016 and/or a heavy chain comprising SEQ ID NO:1046;
(ff) the antibody comprises a light chain comprising SEQ ID NO:1017 and/or a heavy chain comprising SEQ ID NO:1047;
(gg) the antibody comprises a light chain comprising SEQ ID NO:1018 and/or a heavy chain comprising SEQ ID NO:1048;
(hh) the antibody comprises a light chain comprising SEQ ID NO:1019 and/or a heavy chain comprising SEQ ID NO:1049;
(ii) the antibody comprises a light chain comprising SEQ ID NO:1020 and/or a heavy chain comprising SEQ ID NO:1050;
(jj) the antibody comprises a light chain comprising SEQ ID NO:1021 and/or a heavy chain comprising SEQ ID NO:1051;
(kk) the antibody comprises a light chain comprising SEQ ID NO:1022 and/or a heavy chain comprising SEQ ID NO:1052;
(ll) the antibody comprises a light chain comprising SEQ ID NO:236 and/or a heavy chain comprising SEQ ID NO:1053;
(mm) the antibody comprises a light chain comprising SEQ ID NO:242 and/or a heavy chain comprising SEQ ID NO:1054;
(nn) the antibody comprises a light chain comprising SEQ ID NO:243 and/or a heavy chain comprising SEQ ID NO:1055;
(oo) the antibody comprises a light chain comprising SEQ ID NO:245 and/or a heavy chain comprising SEQ ID NO:1056;
(pp) the antibody comprises a light chain comprising SEQ ID NO:1007 and/or a heavy chain comprising SEQ ID NO:1057;
(qq) the antibody comprises a light chain comprising SEQ ID NO:1008 and/or a heavy chain comprising SEQ ID NO:1058;
(rr) the antibody comprises a light chain comprising SEQ ID NO:1012 and/or a heavy chain comprising SEQ ID NO:1059;
(ss) the antibody comprises a light chain comprising SEQ ID NO:1017 and/or a heavy chain comprising SEQ ID NO:1060;
(tt) the antibody comprises a light chain comprising SEQ ID NO:1018 and/or a heavy chain comprising SEQ ID NO:1061;
(uu) the antibody comprises a light chain comprising SEQ ID NO:1019 and/or a heavy chain comprising SEQ ID NO:1062;
(vv) the antibody comprises a light chain comprising SEQ ID NO:1020 and/or a heavy chain comprising SEQ ID NO:1063;
(ww) the antibody comprises a light chain comprising SEQ ID NO:1021 and/or a heavy chain comprising SEQ ID NO:1064;
(xx) the antibody comprises a light chain comprising SEQ ID NO:1022 and/or a heavy chain comprising SEQ ID NO:1065;
(yy) the antibody comprises a light chain comprising SEQ ID NO:236 and/or a heavy chain comprising SEQ ID NO:1070;
(zz) the antibody comprises a light chain comprising SEQ ID NO:237 and/or a heavy chain comprising SEQ ID NO:1071;
(aaa) the antibody comprises a light chain comprising SEQ ID NO:238 and/or a heavy chain comprising SEQ ID NO:1072;
(bbb) the antibody comprises a light chain comprising SEQ ID NO:239 and/or a heavy chain comprising SEQ ID NO:1073;
(ccc) the antibody comprises a light chain comprising SEQ ID NO:240 and/or a heavy chain comprising SEQ ID NO:1074;
(ddd) the antibody comprises a light chain comprising SEQ ID NO:241 and/or a heavy chain comprising SEQ ID NO:1075;
(eee) the antibody comprises a light chain comprising SEQ ID NO:242 and/or a heavy chain comprising SEQ ID NO:1076;
(fff) the antibody comprises a light chain comprising SEQ ID NO:243 and/or a heavy chain comprising SEQ ID NO:1077;
(ggg) the antibody comprises a light chain comprising SEQ ID NO:244 and/or a heavy chain comprising SEQ ID NO:1078;
(hhh) the antibody comprises a light chain comprising SEQ ID NO:245 and/or a heavy chain comprising SEQ ID NO:1079;
(iii) the antibody comprises a light chain comprising SEQ ID NO:246 and/or a heavy chain comprising SEQ ID NO:1080;
(jjj) the antibody comprises a light chain comprising SEQ ID NO:247 and/or a heavy chain comprising SEQ ID NO:1081;
(kkk) the antibody comprises a light chain comprising SEQ ID NO:1000 and/or a heavy chain comprising SEQ ID NO:1082;
(lll) the antibody comprises a light chain comprising SEQ ID NO:1001 and/or a heavy chain comprising SEQ ID NO:1083;
(mmm) the antibody comprises a light chain comprising SEQ ID NO:1002 and/or a heavy chain comprising SEQ ID NO:1084;
(nnn) the antibody comprises a light chain comprising SEQ ID NO:1003 and/or a heavy chain comprising SEQ ID NO:1085;
(ooo) the antibody comprises a light chain comprising SEQ ID NO:1004 and/or a heavy chain comprising SEQ ID NO:1086;
(ppp) the antibody comprises a light chain comprising SEQ ID NO:1005 and/or a heavy chain comprising SEQ ID NO:1087;
(qqq) the antibody comprises a light chain comprising SEQ ID NO:1006 and/or a heavy chain comprising SEQ ID NO:1088;
(rrr) the antibody comprises a light chain comprising SEQ ID NO:1007 and/or a heavy chain comprising SEQ ID NO:1089;
(sss) the antibody comprises a light chain comprising SEQ ID NO:1008 and/or a heavy chain comprising SEQ ID NO:1090;
(ttt) the antibody comprises a light chain comprising SEQ ID NO:1009 and/or a heavy chain comprising SEQ ID NO:1091;
(uuu) the antibody comprises a light chain comprising SEQ ID NO:1010 and/or a heavy chain comprising SEQ ID NO:1092;
(vvv) the antibody comprises a light chain comprising SEQ ID NO:1011 and/or a heavy chain comprising SEQ ID NO:1093;
(www) the antibody comprises a light chain comprising SEQ ID NO:1012 and/or a heavy chain comprising SEQ ID NO:1094;
(xxx) the antibody comprises a light chain comprising SEQ ID NO:1013 and/or a heavy chain comprising SEQ ID NO:1095;
(yyy) the antibody comprises a light chain comprising SEQ ID NO:1014 and/or a heavy chain comprising SEQ ID NO:1096;
(zzz) the antibody comprises a light chain comprising SEQ ID NO:1015 and/or a heavy chain comprising SEQ ID NO:1097;
(aaaa) the antibody comprises a light chain comprising SEQ ID NO:1016 and/or a heavy chain comprising SEQ ID NO:1098;
(bbbb) the antibody comprises a light chain comprising SEQ ID NO:1017 and/or a heavy chain comprising SEQ ID NO:1099;
(cccc) the antibody comprises a light chain comprising SEQ ID NO:1018 and/or a heavy chain comprising SEQ ID NO:1100;
(dddd) the antibody comprises a light chain comprising SEQ ID NO:1019 and/or a heavy chain comprising SEQ ID NO:1101;
(eeee) the antibody comprises a light chain comprising SEQ ID NO:1020 and/or a heavy chain comprising SEQ ID NO:1102;
(ffff) the antibody comprises a light chain comprising SEQ ID NO:1021 and/or a heavy chain comprising SEQ ID NO:1103;
(gggg) the antibody comprises a light chain comprising SEQ ID NO:1022 and/or a heavy chain comprising SEQ ID NO:1104;
(hhhh) the antibody comprises a light chain comprising SEQ ID NO:236 and/or a heavy chain comprising SEQ ID NO:1105;
(iiii) the antibody comprises a light chain comprising SEQ ID NO:242 and/or a heavy chain comprising SEQ ID NO:1106;
(jjjj) the antibody comprises a light chain comprising SEQ ID NO:243 and/or a heavy chain comprising SEQ ID NO:1107;
(kkkk) the antibody comprises a light chain comprising SEQ ID NO:245 and/or a heavy chain comprising SEQ ID NO:1108;
(llll) the antibody comprises a light chain comprising SEQ ID NO:1007 and/or a heavy chain comprising SEQ ID NO:1109;
(mmmm) the antibody comprises a light chain comprising SEQ ID NO:1008 and/or a heavy chain comprising SEQ ID NO:1110;
(nnnn) the antibody comprises a light chain comprising SEQ ID NO:1012 and/or a heavy chain comprising SEQ ID NO:1111;
(oooo) the antibody comprises a light chain comprising SEQ ID NO:1017 and/or a heavy chain comprising SEQ ID NO:1112;
(pppp) the antibody comprises a light chain comprising SEQ ID NO:1018 and/or a heavy chain comprising SEQ ID NO:1113;
(qqqq) the antibody comprises a light chain comprising SEQ ID NO:1019 and/or a heavy chain comprising SEQ ID NO:1114;
(rrrr) the antibody comprises a light chain comprising SEQ ID NO:1020 and/or a heavy chain comprising SEQ ID NO:1115;
(ssss) the antibody comprises a light chain comprising SEQ ID NO:1021 and/or a heavy chain comprising SEQ ID NO:1116; or
(tttt) the antibody comprises a light chain comprising SEQ ID NO:1022 and/or a heavy chain comprising SEQ ID NO:1117.

In one aspect, provided herein is an antibody that specifically binds to human nectin-4, wherein the antibody comprises a light chain variable region (VL) domain and a heavy chain variable region (VH) domain, wherein:
(a) the VL domain comprises CDR-L1, CDR-L2, and CDR-L3 sequences from a VL domain selected from the group consisting of SEQ ID NOs:248, 250, 252, 254, 256, 258, 260, 262, 264, 266, 268, 270, 900, 902, 904, 906, 908, 910, 912, 914, 916, 918, 920, 922, 924, 926, 928, 930, 932, 934, 936, 938, 940, 942, and 944 and the VH domain comprises CDR-H1, CDR-H2, and CDR-H3 sequences from a VH domain selected from the group consisting of SEQ ID NOs:249, 251, 253, 255, 257, 259, 261, 263, 265, 267, 269, 271, 901, 903, 905, 907, 909, 911, 913, 915, 917, 919, 921, 923, 925, 927, 929, 931, 933, 935, 937, 939, 941, 943, and 945;
(b) the VL domain comprises CDR-L1, CDR-L2, and CDR-L3 sequences from a VL domain of SEQ ID NO:248 and the VH domain comprises CDR-H1, CDR-H2, and CDR-H3 from a VH domain of SEQ ID NO:249;
(c) the VL domain comprises CDR-L1, CDR-L2, and CDR-L3 sequences from a VL domain of SEQ ID NO:250 and the VH domain comprises CDR-H1, CDR-H2, and CDR-H3 from a VH domain of SEQ ID NO:251;
(d) the VL domain comprises CDR-L1, CDR-L2, and CDR-L3 sequences from a VL domain of SEQ ID NO:252 and the VH domain comprises CDR-H1, CDR-H2, and CDR-H3 from a VH domain of SEQ ID NO:253;
(e) the VL domain comprises CDR-L1, CDR-L2, and CDR-L3 sequences from a VL domain of SEQ ID NO:254 and the VH domain comprises CDR-H1, CDR-H2, and CDR-H3 from a VH domain of SEQ ID NO:255;
(f) the VL domain comprises CDR-L1, CDR-L2, and CDR-L3 sequences from a VL domain of SEQ ID NO:256 and the VH domain comprises CDR-H1, CDR-H2, and CDR-H3 from a VH domain of SEQ ID NO:257;
(g) the VL domain comprises CDR-L1, CDR-L2, and CDR-L3 sequences from a VL domain of SEQ ID NO:258 and the VH domain comprises CDR-H1, CDR-H2, and CDR-H3 from a VH domain of SEQ ID NO:259;
(h) the VL domain comprises CDR-L1, CDR-L2, and CDR-L3 sequences from a VL domain of SEQ ID NO:260 and the VH domain comprises CDR-H1, CDR-H2, and CDR-H3 from a VH domain of SEQ ID NO:261;
(i) the VL domain comprises CDR-L1, CDR-L2, and CDR-L3 sequences from a VL domain of SEQ ID NO:262 and the VH domain comprises CDR-H1, CDR-H2, and CDR-H3 from a VH domain of SEQ ID NO:263;
(j) the VL domain comprises CDR-L1, CDR-L2, and CDR-L3 sequences from a VL domain of SEQ ID NO:264 and the VH domain comprises CDR-H1, CDR-H2, and CDR-H3 from a VH domain of SEQ ID NO:265;
(k) the VL domain comprises CDR-L1, CDR-L2, and CDR-L3 sequences from a VL domain of SEQ ID NO:266 and the VH domain comprises CDR-H1, CDR-H2, and CDR-H3 from a VH domain of SEQ ID NO:267;
(1) the VL domain comprises CDR-L1, CDR-L2, and CDR-L3 sequences from a VL domain of SEQ ID NO:268 and the VH domain comprises CDR-H1, CDR-H2, and CDR-H3 from a VH domain of SEQ ID NO:269;
(m) the VL domain comprises CDR-L1, CDR-L2, and CDR-L3 sequences from a VL domain of SEQ ID NO:270 and the VH domain comprises CDR-H1, CDR-H2, and CDR-H3 from a VH domain of SEQ ID NO:271
(n) the VL domain comprises CDR-L1, CDR-L2, and CDR-L3 sequences from a VL domain of SEQ ID NO:900 and the VH domain comprises CDR-H1, CDR-H2, and CDR-H3 from a VH domain of SEQ ID NO:901;
(o) the VL domain comprises CDR-L1, CDR-L2, and CDR-L3 sequences from a VL domain of SEQ ID NO:902 and the VH domain comprises CDR-H1, CDR-H2, and CDR-H3 from a VH domain of SEQ ID NO:903;
(p) the VL domain comprises CDR-L1, CDR-L2, and CDR-L3 sequences from a VL domain of SEQ ID NO:904 and the VH domain comprises CDR-H1, CDR-H2, and CDR-H3 from a VH domain of SEQ ID NO:905;
(q) the VL domain comprises CDR-L1, CDR-L2, and CDR-L3 sequences from a VL domain of SEQ ID NO:906 and the VH domain comprises CDR-H1, CDR-H2, and CDR-H3 from a VH domain of SEQ ID NO:907;
(r) the VL domain comprises CDR-L1, CDR-L2, and CDR-L3 sequences from a VL domain of SEQ ID NO:908 and the VH domain comprises CDR-H1, CDR-H2, and CDR-H3 from a VH domain of SEQ ID NO:909;
(s) the VL domain comprises CDR-L1, CDR-L2, and CDR-L3 sequences from a VL domain of SEQ ID NO:910 and the VH domain comprises CDR-H1, CDR-H2, and CDR-H3 from a VH domain of SEQ ID NO:911;
(t) the VL domain comprises CDR-L1, CDR-L2, and CDR-L3 sequences from a VL domain of SEQ ID NO:912 and the VH domain comprises CDR-H1, CDR-H2, and CDR-H3 from a VH domain of SEQ ID NO:913;
(u) the VL domain comprises CDR-L1, CDR-L2, and CDR-L3 sequences from a VL domain of SEQ ID NO:914 and the VH domain comprises CDR-H1, CDR-H2, and CDR-H3 from a VH domain of SEQ ID NO:915;
(v) the VL domain comprises CDR-L1, CDR-L2, and CDR-L3 sequences from a VL domain of SEQ ID NO:916 and the VH domain comprises CDR-H1, CDR-H2, and CDR-H3 from a VH domain of SEQ ID NO:917;
(w) the VL domain comprises CDR-L1, CDR-L2, and CDR-L3 sequences from a VL domain of SEQ ID NO:918 and the VH domain comprises CDR-H1, CDR-H2, and CDR-H3 from a VH domain of SEQ ID NO:919;
(x) the VL domain comprises CDR-L1, CDR-L2, and CDR-L3 sequences from a VL domain of SEQ ID NO:920 and the VH domain comprises CDR-H1, CDR-H2, and CDR-H3 from a VH domain of SEQ ID NO:921;
(y) the VL domain comprises CDR-L1, CDR-L2, and CDR-L3 sequences from a VL domain of SEQ ID NO:922 and the VH domain comprises CDR-H1, CDR-H2, and CDR-H3 from a VH domain of SEQ ID NO:923;
(z) the VL domain comprises CDR-L1, CDR-L2, and CDR-L3 sequences from a VL domain of SEQ ID NO:924 and the VH domain comprises CDR-H1, CDR-H2, and CDR-H3 from a VH domain of SEQ ID NO:925;
(aa) the VL domain comprises CDR-L1, CDR-L2, and CDR-L3 sequences from a VL domain of SEQ ID NO:926 and the VH domain comprises CDR-H1, CDR-H2, and CDR-H3 from a VH domain of SEQ ID NO:927;
(bb) the VL domain comprises CDR-L1, CDR-L2, and CDR-L3 sequences from a VL domain of SEQ ID NO:928 and the VH domain comprises CDR-H1, CDR-H2, and CDR-H3 from a VH domain of SEQ ID NO:929;
(cc) the VL domain comprises CDR-L1, CDR-L2, and CDR-L3 sequences from a VL domain of SEQ ID NO:930 and the VH domain comprises CDR-H1, CDR-H2, and CDR-H3 from a VH domain of SEQ ID NO:931;
(dd) the VL domain comprises CDR-L1, CDR-L2, and CDR-L3 sequences from a VL domain of SEQ ID NO932; and the VH domain comprises CDR-H1, CDR-H2, and CDR-H3 from a VH domain of SEQ ID NO:933;
(ee) the VL domain comprises CDR-L1, CDR-L2, and CDR-L3 sequences from a VL domain of SEQ ID NO:934 and the VH domain comprises CDR-H1, CDR-H2, and CDR-H3 from a VH domain of SEQ ID NO:935;
(ff) the VL domain comprises CDR-L1, CDR-L2, and CDR-L3 sequences from a VL domain of SEQ ID NO:936 and the VH domain comprises CDR-H1, CDR-H2, and CDR-H3 from a VH domain of SEQ ID NO:937;
(gg) the VL domain comprises CDR-L1, CDR-L2, and CDR-L3 sequences from a VL domain of SEQ ID NO:938 and the VH domain comprises CDR-H1, CDR-H2, and CDR-H3 from a VH domain of SEQ ID NO:939;
(hh) the VL domain comprises CDR-L1, CDR-L2, and CDR-L3 sequences from a VL domain of SEQ ID NO:940 and the VH domain comprises CDR-H1, CDR-H2, and CDR-H3 from a VH domain of SEQ ID NO:941;
(ii) the VL domain comprises CDR-L1, CDR-L2, and CDR-L3 sequences from a VL domain of SEQ ID NO:942 and the VH domain comprises CDR-H1, CDR-H2, and CDR-H3 from a VH domain of SEQ ID NO:943; or
(jj) the VL domain comprises CDR-L1, CDR-L2, and CDR-L3 sequences from a VL domain of SEQ ID NO:944 and the VH domain comprises CDR-H1, CDR-H2, and CDR-H3 from a VH domain of SEQ ID NO:945.

In some embodiments of the antibody:
(a) the VH domain comprises CDR-H1, CDR-H2, and CDR-H3 and the VL domain comprises CDR-L1, CDR-L2, and CDR-L3, wherein CDR-H1 comprises SEQ ID NO:275, CDR-H2 comprises SEQ ID NO:276, CDR-H3 comprises SEQ ID NO:277, CDR-L1 comprises SEQ ID NO:272, CDR-L2 comprises SEQ ID NO:273, and CDR-L3 comprises SEQ ID NO:274;
(b) the VH domain comprises CDR-H1, CDR-H2, and CDR-H3 and the VL domain comprises CDR-L1, CDR-L2, and CDR-L3, wherein CDR-H1 comprises SEQ ID NO:293, CDR-H2 comprises SEQ ID NO:294, CDR-H3 comprises SEQ ID NO:295, CDR-L1 comprises SEQ ID NO:290, CDR-L2 comprises SEQ ID NO:291, and CDR-L3 comprises SEQ ID NO:292;
(c) the VH domain comprises CDR-H1, CDR-H2, and CDR-H3 and the VL domain comprises CDR-L1, CDR-L2, and CDR-L3, wherein CDR-H1 comprises SEQ ID NO:311, CDR-H2 comprises SEQ ID NO:312, CDR-H3 comprises SEQ ID NO:313, CDR-L1 comprises SEQ ID NO:308, CDR-L2 comprises SEQ ID NO:309, and CDR-L3 comprises SEQ ID NO:310;
(d) the VH domain comprises CDR-H1, CDR-H2, and CDR-H3 and the VL domain comprises CDR-L1, CDR-L2, and CDR-L3, wherein CDR-H1 comprises SEQ ID NO:329, CDR-H2 comprises SEQ ID NO:330, CDR-H3 comprises SEQ ID NO:331, CDR-L1 comprises SEQ ID NO:326, CDR-L2 comprises SEQ ID NO:327, and CDR-L3 comprises SEQ ID NO:328;
(e) the VH domain comprises CDR-H1, CDR-H2, and CDR-H3 and the VL domain comprises CDR-L1, CDR-L2, and CDR-L3, wherein CDR-H1 comprises SEQ ID NO:347, CDR-H2 comprises SEQ ID NO:348, CDR-H3 comprises SEQ ID NO:349, CDR-L1 comprises SEQ ID NO:344, CDR-L2 comprises SEQ ID NO:345, and CDR-L3 comprises SEQ ID NO:346;
(f) the VH domain comprises CDR-H1, CDR-H2, and CDR-H3 and the VL domain comprises CDR-L1, CDR-L2, and CDR-L3, wherein CDR-H1 comprises SEQ ID NO:365, CDR-H2 comprises SEQ ID NO:366, CDR-H3 comprises SEQ ID NO:367, CDR-L1 comprises SEQ ID NO:362, CDR-L2 comprises SEQ ID NO:363, and CDR-L3 comprises SEQ ID NO:364;
(g) the VH domain comprises CDR-H1, CDR-H2, and CDR-H3 and the VL domain comprises CDR-L1, CDR-L2, and CDR-L3, wherein CDR-H1 comprises SEQ ID NO:383, CDR-H2 comprises SEQ ID NO:384, CDR-H3 comprises SEQ ID NO:385, CDR-L1 comprises SEQ ID NO:380, CDR-L2 comprises SEQ ID NO:381, and CDR-L3 comprises SEQ ID NO:382;
(h) the VH domain comprises CDR-H1, CDR-H2, and CDR-H3 and the VL domain comprises CDR-L1, CDR-L2, and CDR-L3, wherein CDR-H1 comprises SEQ ID NO:401, CDR-H2 comprises SEQ ID NO:402, CDR-H3 comprises SEQ ID NO:403, CDR-L1 comprises SEQ ID NO:398, CDR-L2 comprises SEQ ID NO:399, and CDR-L3 comprises SEQ ID NO:400;
(i) the VH domain comprises CDR-H1, CDR-H2, and CDR-H3 and the VL domain comprises CDR-L1, CDR-L2, and CDR-L3, wherein CDR-H1 comprises SEQ ID NO:419, CDR-H2 comprises SEQ ID NO:420, CDR-H3 comprises SEQ ID NO:421, CDR-L1 comprises SEQ ID NO:416, CDR-L2 comprises SEQ ID NO:417, and CDR-L3 comprises SEQ ID NO:418;
(j) the VH domain comprises CDR-H1, CDR-H2, and CDR-H3 and the VL domain comprises CDR-L1, CDR-L2, and CDR-L3, wherein CDR-H1 comprises SEQ ID NO:437, CDR-H2 comprises SEQ ID NO:438, CDR-H3 comprises SEQ ID NO:439, CDR-L1 comprises SEQ ID NO:434, CDR-L2 comprises SEQ ID NO:435, and CDR-L3 comprises SEQ ID NO:436;
(k) the VH domain comprises CDR-H1, CDR-H2, and CDR-H3 and the VL domain comprises CDR-L1, CDR-L2, and CDR-L3, wherein CDR-H1 comprises SEQ ID NO:455, CDR-H2 comprises SEQ ID NO:456, CDR-H3 comprises SEQ ID NO:457, CDR-L1 comprises SEQ ID NO:452, CDR-L2 comprises SEQ ID NO:453, and CDR-L3 comprises SEQ ID NO:454;
(1) the VH domain comprises CDR-H1, CDR-H2, and CDR-H3 and the VL domain comprises CDR-L1, CDR-L2, and CDR-L3, wherein CDR-H1 comprises SEQ ID NO:281, CDR-H2 comprises SEQ ID NO:282, CDR-H3 comprises SEQ ID NO:283, CDR-L1 comprises SEQ ID NO:278, CDR-L2 comprises SEQ ID NO:279, and CDR-L3 comprises SEQ ID NO:280;
(m) the VH domain comprises CDR-H1, CDR-H2, and CDR-H3 and the VL domain comprises CDR-L1, CDR-L2, and CDR-L3, wherein CDR-H1 comprises SEQ ID NO:299, CDR-H2 comprises SEQ ID NO:300, CDR-H3 comprises SEQ ID NO:301, CDR-L1 comprises SEQ ID NO:296, CDR-L2 comprises SEQ ID NO:297, and CDR-L3 comprises SEQ ID NO:298;
(n) the VH domain comprises CDR-H1, CDR-H2, and CDR-H3 and the VL domain comprises CDR-L1, CDR-L2, and CDR-L3, wherein CDR-H1 comprises SEQ ID NO:317, CDR-H2 comprises SEQ ID NO:318, CDR-H3 comprises SEQ ID NO:319, CDR-L1 comprises SEQ ID NO:314, CDR-L2 comprises SEQ ID NO:315, and CDR-L3 comprises SEQ ID NO:316;
(o) the VH domain comprises CDR-H1, CDR-H2, and CDR-H3 and the VL domain comprises CDR-L1, CDR-L2, and CDR-L3, wherein CDR-H1 comprises SEQ ID NO:335, CDR-H2 comprises SEQ ID NO:336, CDR-H3 comprises SEQ ID NO:337, CDR-L1 comprises SEQ ID NO:332, CDR-L2 comprises SEQ ID NO:333, and CDR-L3 comprises SEQ ID NO:334;
(p) the VH domain comprises CDR-H1, CDR-H2, and CDR-H3 and the VL domain comprises CDR-L1, CDR-L2, and CDR-L3, wherein CDR-H1 comprises SEQ ID NO:353, CDR-H2 comprises SEQ ID NO:354, CDR-H3 comprises SEQ ID NO:355, CDR-L1 comprises SEQ ID NO:350, CDR-L2 comprises SEQ ID NO:351, and CDR-L3 comprises SEQ ID NO:352;
(q) the VH domain comprises CDR-H1, CDR-H2, and CDR-H3 and the VL domain comprises CDR-L1, CDR-L2, and CDR-L3, wherein CDR-H1 comprises SEQ ID NO:371, CDR-H2 comprises SEQ ID NO:372, CDR-H3 comprises SEQ ID NO:373, CDR-L1 comprises SEQ ID NO:368, CDR-L2 comprises SEQ ID NO:369, and CDR-L3 comprises SEQ ID NO:370;
(r) the VH domain comprises CDR-H1, CDR-H2, and CDR-H3 and the VL domain comprises CDR-L1, CDR-L2, and CDR-L3, wherein CDR-H1 comprises SEQ ID NO:389, CDR-H2 comprises SEQ ID NO:390, CDR-H3 comprises SEQ ID NO:391, CDR-L1 comprises SEQ ID NO:386, CDR-L2 comprises SEQ ID NO:387, and CDR-L3 comprises SEQ ID NO:388;
(s) the VH domain comprises CDR-H1, CDR-H2, and CDR-H3 and the VL domain comprises CDR-L1, CDR-L2, and CDR-L3, wherein CDR-H1 comprises SEQ ID NO:407, CDR-H2 comprises SEQ ID NO:408, CDR-H3 comprises SEQ ID NO:409, CDR-L1 comprises SEQ ID NO:404, CDR-L2 comprises SEQ ID NO:405, and CDR-L3 comprises SEQ ID NO:406;
(t) the VH domain comprises CDR-H1, CDR-H2, and CDR-H3 and the VL domain comprises CDR-L1, CDR-L2, and CDR-L3, wherein CDR-H1 comprises SEQ ID NO:425, CDR-H2 comprises SEQ ID NO:426, CDR-H3 comprises SEQ ID NO:427, CDR-L1 comprises SEQ ID NO:422, CDR-L2 comprises SEQ ID NO:423, and CDR-L3 comprises SEQ ID NO:424;
(u) the VH domain comprises CDR-H1, CDR-H2, and CDR-H3 and the VL domain comprises CDR-L1, CDR-L2, and CDR-L3, wherein CDR-H1 comprises SEQ ID NO:443, CDR-H2 comprises SEQ ID NO:444, CDR-H3 comprises SEQ ID NO:445, CDR-L1 comprises SEQ ID NO:440, CDR-L2 comprises SEQ ID NO:441, and CDR-L3 comprises SEQ ID NO:442;
(v) the VH domain comprises CDR-H1, CDR-H2, and CDR-H3 and the VL domain comprises CDR-L1, CDR-L2, and CDR-L3, wherein CDR-H1 comprises SEQ ID NO:461, CDR-H2 comprises SEQ ID NO:462, CDR-H3 comprises SEQ ID NO:463, CDR-L1 comprises SEQ ID NO:458, CDR-L2 comprises SEQ ID NO:459, and CDR-L3 comprises SEQ ID NO:460;
(w) the VH domain comprises CDR-H1, CDR-H2, and CDR-H3 and the VL domain comprises CDR-L1, CDR-L2, and CDR-L3, wherein CDR-H1 comprises SEQ ID NO:287, CDR-H2 comprises SEQ ID NO:288, CDR-H3 comprises SEQ ID NO:289, CDR-L1 comprises SEQ ID NO:284, CDR-L2 comprises SEQ ID NO:285, and CDR-L3 comprises SEQ ID NO:286;
(x) the VH domain comprises CDR-H1, CDR-H2, and CDR-H3 and the VL domain comprises CDR-L1, CDR-L2, and CDR-L3, wherein CDR-H1 comprises SEQ ID NO:305, CDR-H2 comprises SEQ ID NO:306, CDR-H3 comprises SEQ ID NO:307, CDR-L1 comprises SEQ ID NO:302, CDR-L2 comprises SEQ ID NO:303, and CDR-L3 comprises SEQ ID NO:304;
(y) the VH domain comprises CDR-H1, CDR-H2, and CDR-H3 and the VL domain comprises CDR-L1, CDR-L2, and CDR-L3, wherein CDR-H1 comprises SEQ ID NO:323, CDR-H2 comprises SEQ ID NO:324, CDR-H3 comprises SEQ ID NO:325, CDR-L1 comprises SEQ ID NO:320, CDR-L2 comprises SEQ ID NO:321, and CDR-L3 comprises SEQ ID NO:322;
(z) the VH domain comprises CDR-H1, CDR-H2, and CDR-H3 and the VL domain comprises CDR-L1, CDR-L2, and CDR-L3, wherein CDR-H1 comprises SEQ ID NO:341, CDR-H2 comprises SEQ ID NO:342, CDR-H3 comprises SEQ ID NO:343, CDR-L1 comprises SEQ ID NO:338, CDR-L2 comprises SEQ ID NO:339, and CDR-L3 comprises SEQ ID NO:340;
(aa) the VH domain comprises CDR-H1, CDR-H2, and CDR-H3 and the VL domain comprises CDR-L1, CDR-L2, and CDR-L3, wherein CDR-H1 comprises SEQ ID NO:359, CDR-H2 comprises SEQ ID NO:360, CDR-H3 comprises SEQ ID NO:361, CDR-L1 comprises SEQ ID NO:356, CDR-L2 comprises SEQ ID NO:357, and CDR-L3 comprises SEQ ID NO:358;
(bb) the VH domain comprises CDR-H1, CDR-H2, and CDR-H3 and the VL domain comprises CDR-L1, CDR-L2, and CDR-L3, wherein CDR-H1 comprises SEQ ID NO:377, CDR-H2 comprises SEQ ID NO:378, CDR-H3 comprises SEQ ID NO:379, CDR-L1 comprises SEQ ID NO:374, CDR-L2 comprises SEQ ID NO:375, and CDR-L3 comprises SEQ ID NO:376;
(cc) the VH domain comprises CDR-H1, CDR-H2, and CDR-H3 and the VL domain comprises CDR-L1, CDR-L2, and CDR-L3, wherein CDR-H1 comprises SEQ ID NO:395, CDR-H2 comprises SEQ ID NO:396, CDR-H3 comprises SEQ ID NO:397, CDR-L1 comprises SEQ ID NO:392, CDR-L2 comprises SEQ ID NO:393, and CDR-L3 comprises SEQ ID NO:394;
(dd) the VH domain comprises CDR-H1, CDR-H2, and CDR-H3 and the VL domain comprises CDR-L1, CDR-L2, and CDR-L3, wherein CDR-H1 comprises SEQ ID NO:413, CDR-H2 comprises SEQ ID NO:414, CDR-H3 comprises SEQ ID NO:415, CDR-L1 comprises SEQ ID NO:410, CDR-L2 comprises SEQ ID NO:411, and CDR-L3 comprises SEQ ID NO:412;
(ee) the VH domain comprises CDR-H1, CDR-H2, and CDR-H3 and the VL domain comprises CDR-L1, CDR-L2, and CDR-L3, wherein CDR-H1 comprises SEQ ID NO:431, CDR-H2 comprises SEQ ID NO:432, CDR-H3 comprises SEQ ID NO:433, CDR-L1 comprises SEQ ID NO:428, CDR-L2 comprises SEQ ID NO:429, and CDR-L3 comprises SEQ ID NO:430;
(ff) the VH domain comprises CDR-H1, CDR-H2, and CDR-H3 and the VL domain comprises CDR-L1, CDR-L2, and CDR-L3, wherein CDR-H1 comprises SEQ ID NO:449, CDR-H2 comprises SEQ ID NO:450, CDR-H3 comprises SEQ ID NO:451, CDR-L1 comprises SEQ ID NO:446, CDR-L2 comprises SEQ ID NO:447, and CDR-L3 comprises SEQ ID NO:448;
(gg) the VH domain comprises CDR-H1, CDR-H2, and CDR-H3 and the VL domain comprises CDR-L1, CDR-L2, and CDR-L3, wherein CDR-H1 comprises SEQ ID NO:467, CDR-H2 comprises SEQ ID NO:468, CDR-H3 comprises SEQ ID NO:469, CDR-L1 comprises SEQ ID NO:464, CDR-L2 comprises SEQ ID NO:465, and CDR-L3 comprises SEQ ID NO:466;
(hh) the VH domain comprises CDR-H1, CDR-H2, and CDR-H3 and the VL domain comprises CDR-L1, CDR-L2, and CDR-L3, wherein CDR-H1 comprises SEQ ID NO:479, CDR-H2 comprises SEQ ID NO:480, CDR-H3 comprises SEQ ID NO:481, CDR-L1 comprises SEQ ID NO:476, CDR-L2 comprises SEQ ID NO:477, and CDR-L3 comprises SEQ ID NO:478;
(ii) the VH domain comprises CDR-H1, CDR-H2, and CDR-H3 and the VL domain comprises CDR-L1, CDR-L2, and CDR-L3, wherein CDR-H1 comprises SEQ ID NO:485, CDR-H2 comprises SEQ ID NO:486, CDR-H3 comprises SEQ ID NO:487, CDR-L1 comprises SEQ ID NO:482, CDR-L2 comprises SEQ ID NO:483, and CDR-L3 comprises SEQ ID NO:484;
(jj) the VH domain comprises CDR-H1, CDR-H2, and CDR-H3 and the VL domain comprises CDR-L1, CDR-L2, and CDR-L3, wherein CDR-H1 comprises SEQ ID NO:473, CDR-H2 comprises SEQ ID NO:474, CDR-H3 comprises SEQ ID NO:475, CDR-L1 comprises SEQ ID NO:470, CDR-L2 comprises SEQ ID NO:471, and CDR-L3 comprises SEQ ID NO:472;
(kk) the VL domain comprises CDR-L1, CDR-L2, and CDR-L3 and the VH domain comprises CDR-H1, CDR-H2, and CDR-H3, wherein CDR-L1 comprises SEQ ID NO:562, CDR-L2 comprises SEQ ID NO:563, and CDR-L3 comprises SEQ ID NO:564, wherein CDR-H1 comprises SEQ ID NO:565, CDR-H2 comprises SEQ ID NO:566, and CDR-H3 comprises SEQ ID NO:567;
(ll) the VL domain comprises CDR-L1, CDR-L2, and CDR-L3 and the VH domain comprises CDR-H1, CDR-H2, and CDR-H3, wherein CDR-L1 comprises SEQ ID NO:580, CDR-L2 comprises SEQ ID NO:581, and CDR-L3 comprises SEQ ID NO:582, wherein CDR-H1 comprises SEQ ID NO:583, CDR-H2 comprises SEQ ID NO:584, and CDR-H3 comprises SEQ ID NO:585;
(mm) the VL domain comprises CDR-L1, CDR-L2, and CDR-L3 and the VH domain comprises CDR-H1, CDR-H2, and CDR-H3, wherein CDR-L1 comprises SEQ ID NO:598, CDR-L2 comprises SEQ ID NO:599, and CDR-L3 comprises SEQ ID NO:600, wherein CDR-H1 comprises SEQ ID NO:601, CDR-H2 comprises SEQ ID NO:602, and CDR-H3 comprises SEQ ID NO:603;
(nn) the VL domain comprises CDR-L1, CDR-L2, and CDR-L3 and the VH domain comprises CDR-H1, CDR-H2, and CDR-H3, wherein CDR-L1 comprises SEQ ID NO:616, CDR-L2 comprises SEQ ID NO:617, and CDR-L3 comprises SEQ ID NO:618, wherein CDR-H1 comprises SEQ ID NO:619, CDR-H2 comprises SEQ ID NO:620, and CDR-H3 comprises SEQ ID NO:621;
(oo) the VL domain comprises CDR-L1, CDR-L2, and CDR-L3 and the VH domain comprises CDR-H1, CDR-H2, and CDR-H3, wherein CDR-L1 comprises SEQ ID NO:634, CDR-L2 comprises SEQ ID NO:635, and CDR-L3 comprises SEQ ID NO:636, wherein CDR-H1 comprises SEQ ID NO:637, CDR-H2 comprises SEQ ID NO:638, and CDR-H3 comprises SEQ ID NO:639;
(pp) the VL domain comprises CDR-L1, CDR-L2, and CDR-L3 and the VH domain comprises CDR-H1, CDR-H2, and CDR-H3, wherein CDR-L1 comprises SEQ ID NO:652, CDR-L2 comprises SEQ ID NO:653, and CDR-L3 comprises SEQ ID NO:654, wherein CDR-H1 comprises SEQ ID NO:655, CDR-H2 comprises SEQ ID NO:656, and CDR-H3 comprises SEQ ID NO:657;
(qq) the VL domain comprises CDR-L1, CDR-L2, and CDR-L3 and the VH domain comprises CDR-H1, CDR-H2, and CDR-H3, wherein CDR-L1 comprises SEQ ID NO:670, CDR-L2 comprises SEQ ID NO:671, and CDR-L3 comprises SEQ ID NO:672, wherein CDR-H1 comprises SEQ ID NO:673, CDR-H2 comprises SEQ ID NO:674, and CDR-H3 comprises SEQ ID NO:675;
(rr) the VL domain comprises CDR-L1, CDR-L2, and CDR-L3 and the VH domain comprises CDR-H1, CDR-H2, and CDR-H3, wherein CDR-L1 comprises SEQ ID NO:688, CDR-L2 comprises SEQ ID NO:689, and CDR-L3 comprises SEQ ID NO:690, wherein CDR-H1 comprises SEQ ID NO:691, CDR-H2 comprises SEQ ID NO:692, and CDR-H3 comprises SEQ ID NO:693;
(ss) the VL domain comprises CDR-L1, CDR-L2, and CDR-L3 and the VH domain comprises CDR-H1, CDR-H2, and CDR-H3, wherein CDR-L1 comprises SEQ ID NO:706, CDR-L2 comprises SEQ ID NO:707, and CDR-L3 comprises SEQ ID NO:708, wherein CDR-H1 comprises SEQ ID NO:709, CDR-H2 comprises SEQ ID NO:710, and CDR-H3 comprises SEQ ID NO:711;
(tt) the VL domain comprises CDR-L1, CDR-L2, and CDR-L3 and the VH domain comprises CDR-H1, CDR-H2, and CDR-H3, wherein CDR-L1 comprises SEQ ID NO:724, CDR-L2 comprises SEQ ID NO:725, and CDR-L3 comprises SEQ ID NO:726, wherein CDR-H1 comprises SEQ ID NO:727, CDR-H2 comprises SEQ ID NO:728, and CDR-H3 comprises SEQ ID NO:729;
(uu) the VL domain comprises CDR-L1, CDR-L2, and CDR-L3 and the VH domain comprises CDR-H1, CDR-H2, and CDR-H3, wherein CDR-L1 comprises SEQ ID NO:742, CDR-L2 comprises SEQ ID NO:743, and CDR-L3 comprises SEQ ID NO:744, wherein CDR-H1 comprises SEQ ID NO:745, CDR-H2 comprises SEQ ID NO:746, and CDR-H3 comprises SEQ ID NO:747;
(vv) the VL domain comprises CDR-L1, CDR-L2, and CDR-L3 and the VH domain comprises CDR-H1, CDR-H2, and CDR-H3, wherein CDR-L1 comprises SEQ ID NO:760, CDR-L2 comprises SEQ ID NO:761, and CDR-L3 comprises SEQ ID NO:762, wherein CDR-H1 comprises SEQ ID NO:763, CDR-H2 comprises SEQ ID NO:764, and CDR-H3 comprises SEQ ID NO:765;
(ww) the VL domain comprises CDR-L1, CDR-L2, and CDR-L3 and the VH domain comprises CDR-H1, CDR-H2, and CDR-H3, wherein CDR-L1 comprises SEQ ID NO:778, CDR-L2 comprises SEQ ID NO:779, and CDR-L3 comprises SEQ ID NO:780, wherein CDR-H1 comprises SEQ ID NO:781, CDR-H2 comprises SEQ ID NO:782, and CDR-H3 comprises SEQ ID NO:783;
(xx) the VL domain comprises CDR-L1, CDR-L2, and CDR-L3 and the VH domain comprises CDR-H1, CDR-H2, and CDR-H3, wherein CDR-L1 comprises SEQ ID NO:796, CDR-L2 comprises SEQ ID NO:797, and CDR-L3 comprises SEQ ID NO:798, wherein CDR-H1 comprises SEQ ID NO:799, CDR-H2 comprises SEQ ID NO:800, and CDR-H3 comprises SEQ ID NO:801;
(yy) the VL domain comprises CDR-L1, CDR-L2, and CDR-L3 and the VH domain comprises CDR-H1, CDR-H2, and CDR-H3, wherein CDR-L1 comprises SEQ ID NO:814, CDR-L2 comprises SEQ ID NO:815, and CDR-L3 comprises SEQ ID NO:816, wherein CDR-H1 comprises SEQ ID NO:817, CDR-H2 comprises SEQ ID NO:818, and CDR-H3 comprises SEQ ID NO:819; or
(zz) the VL domain comprises CDR-L1, CDR-L2, and CDR-L3 and the VH domain comprises CDR-H1, CDR-H2, and CDR-H3, wherein CDR-L1 comprises SEQ ID NO:832, CDR-L2 comprises SEQ ID NO:833, and CDR-L3 comprises SEQ ID NO:834, wherein CDR-H1 comprises SEQ ID NO:835, CDR-H2 comprises SEQ ID NO:836, and CDR-H3 comprises SEQ ID NO:837.

In some embodiments of the antibody:
(a) the VH domain comprises the sequence of amino acids selected from the group consisting of SEQ ID NOs:249, 251, 253, 255, 257, 259, 261, 263, 265, 267, 269, 271, 901, 903, 905, 907, 909, 911, 913, 915, 917, 919, 921, 923, 925, 927, 929, 931, 933, 935, 937, 939, 941, 943, and 945 and/or the VL domain comprises the sequence of amino acids selected from the group consisting of SEQ ID NOs: 248, 250, 252, 254, 256, 258, 260, 262, 264, 266, 268, 270, 900, 902, 904, 906, 908, 910, 912, 914, 916, 918, 920, 922, 924, 926, 928, 930, 932, 934, 936, 938, 940, 942, and 944;
(b) the VL domain comprises the sequence of SEQ ID NO:248 and/or the VH domain comprises the sequence of SEQ ID NO:249;
(c) the VL domain comprises the sequence of SEQ ID NO:250 and/or the VH domain comprises the sequence of SEQ ID NO:251;
(d) the VL domain comprises the sequence of SEQ ID NO:252 and/or the VH domain comprises the sequence of SEQ ID NO:253;
(e) the VL domain comprises the sequence of SEQ ID NO:254 and/or the VH domain comprises the sequence of SEQ ID NO:255;
(f) the VL domain comprises the sequence of SEQ ID NO:256 and/or the VH domain comprises the sequence of SEQ ID NO:257;
(g) the VL domain comprises the sequence of SEQ ID NO:258 and/or the VH domain comprises the sequence of SEQ ID NO:259;
(h) the VL domain comprises the sequence of SEQ ID NO:260 and/or the VH domain comprises the sequence of SEQ ID NO:261;
(i) the VL domain comprises the sequence of SEQ ID NO:262 and/or the VH domain comprises the sequence of SEQ ID NO:263;
(j) the VL domain comprises the sequence of SEQ ID NO:264 and/or the VH domain comprises the sequence of SEQ ID NO:265;
(k) the VL domain comprises the sequence of SEQ ID NO:266 and/or the VH domain comprises the sequence of SEQ ID NO:267;
(l) the VL domain comprises the sequence of SEQ ID NO:268 and/or the VH domain comprises the sequence of SEQ ID NO:269;
(m) the VL domain comprises the sequence of SEQ ID NO:270 and/or the VH domain comprises the sequence of SEQ ID NO:271;
(n) the VL domain comprises the sequence of SEQ ID NO:900 and/or the VH domain comprises the sequence of SEQ ID NO:901;
(o) the VL domain comprises the sequence of SEQ ID NO:902 and/or the VH domain comprises the sequence of SEQ ID NO:903;
(p) the VL domain comprises the sequence of SEQ ID NO:904 and/or the VH domain comprises the sequence of SEQ ID NO:905;
(q) the VL domain comprises the sequence of SEQ ID NO:906 and/or the VH domain comprises the sequence of SEQ ID NO:907;
(r) the VL domain comprises the sequence of SEQ ID NO:908 and/or the VH domain comprises the sequence of SEQ ID NO:909;
(s) the VL domain comprises the sequence of SEQ ID NO:910 and/or the VH domain comprises the sequence of SEQ ID NO:911;
(t) the VL domain comprises the sequence of SEQ ID NO:912 and/or the VH domain comprises the sequence of SEQ ID NO:913;
(u) the VL domain comprises the sequence of SEQ ID NO:914 and/or the VH domain comprises the sequence of SEQ ID NO:915;
(v) the VL domain comprises the sequence of SEQ ID NO:916 and/or the VH domain comprises the sequence of SEQ ID NO:917;
(w) the VL domain comprises the sequence of SEQ ID NO:918 and/or the VH domain comprises the sequence of SEQ ID NO:919;
(x) the VL domain comprises the sequence of SEQ ID NO:920 and/or the VH domain comprises the sequence of SEQ ID NO:921;
(y) the VL domain comprises the sequence of SEQ ID NO:922 and/or the VH domain comprises the sequence of SEQ ID NO:923;
(z) the VL domain comprises the sequence of SEQ ID NO:924 and/or the VH domain comprises the sequence of SEQ ID NO:925;
(aa) the VL domain comprises the sequence of SEQ ID NO:926 and/or the VH domain comprises the sequence of SEQ ID NO:927;
(bb) the VL domain comprises the sequence of SEQ ID NO:928 and/or the VH domain comprises the sequence of SEQ ID NO:929;
(cc) the VL domain comprises the sequence of SEQ ID NO:930 and/or the VH domain comprises the sequence of SEQ ID NO:931;
(dd) the VL domain comprises the sequence of SEQ ID NO:932 and/or the VH domain comprises the sequence of SEQ ID NO:933;
(ee) the VL domain comprises the sequence of SEQ ID NO:934 and/or the VH domain comprises the sequence of SEQ ID NO:935;
(ff) the VL domain comprises the sequence of SEQ ID NO:936 and/or the VH domain comprises the sequence of SEQ ID NO:937;
(gg) the VL domain comprises the sequence of SEQ ID NO:938 and/or the VH domain comprises the sequence of SEQ ID NO:939;
(hh) the VL domain comprises the sequence of SEQ ID NO:940 and/or the VH domain comprises the sequence of SEQ ID NO:941;
(ii) the VL domain comprises the sequence of SEQ ID NO:942 and/or the VH domain comprises the sequence of SEQ ID NO:943; or
(jj) the VL domain comprises the sequence of SEQ ID NO:944 and/or the VH domain comprises the sequence of SEQ ID NO:945.

In some embodiments, the antibody is linked to one or more Q-tag peptides (Q) comprising at least one glutamine residue. In some embodiments, antibody comprises two antibody heavy chains and two antibody light chains, and wherein one Q-tag is attached to one or both heavy chain(s) of the antibody. In some embodiments, the Q-tag is fused to the C-terminus of the heavy chain of the antibody. In some embodiments, each Q-tag independently comprises a peptide sequence comprising between 5 and 15 amino acid residues. In some embodiments, the Q-tag is naturally occurring. In some embodiments, the peptide sequence of each Q-tag is independently selected from the group consisting of SEQ ID NOs:39-55. In some embodiments, the Q-tag comprises the peptide sequence RPQGF (SEQ ID NO:47). In some embodiments, each Q-tag independently comprises RPQGF (SEQ ID NO:47), RPQGFPP (SEQ ID NO:48), or RPQGFGPP (SEQ ID NO:49). In some embodiments, 1 or 2 Q-tags is/are linked to the antibody. In some embodiments, the antibody further comprises an amino acid sequence selected from the group consisting of SEQ ID Nos:92-107, 111, 112, 178, and 494-497.

In some embodiments, the antibody is a Fab, F(ab′)2, Fab′-SH, Fv, scFv, single domain, single heavy chain, or single light chain antibody or antibody fragment. In some embodiments, the antibody comprises an Fc region. In some embodiments, the Fc region is a human Fc region selected from the group consisting of an IgG1 Fc region, an IgG2 Fc region, and an IgG4 Fc region. In some embodiments, the Fc region is a human Fc region comprising one or more amino acid substitutions that reduce binding to C1q. In some embodiments, the Fc region is a human Fc region comprising one or more amino acid substitutions that increase binding to C1q. In some embodiments, the Fc region is a human Fc region exhibiting wild-type complement activation. In some embodiments, the Fc region is a human Fc region comprising one or more amino acid substitutions that increase complement activation. In some embodiments, the Fc region is a human Fc region comprising one or more amino acid substitutions and exhibiting wild-type binding to C1q. In some embodiments, the Fc region is a human Fc region comprising one or more amino acid substitutions that reduce effector function, as compared with a human Fc region that lacks the amino acid substitution(s). In some embodiments, the Fc region is: (a) a human IgG1 Fc region comprising L234A, L235A, and/or G237A substitutions, amino acid position numbering according to EU index; (b) a human IgG2 Fc region comprising A330S and/or P331S substitutions, amino acid position numbering according to EU index; or (c) a human IgG4 Fc region comprising S228P and/or L235E substitutions, amino acid position numbering according to EU index. In some embodiments, the Fc region further comprises an N297A substitution, amino acid position numbering according to EU index. In some embodiments, the Fc region is a wild-type human IgG1 Fc region. In some embodiments, the Fc region is a human IgG1 Fc region that has been engineered to improve antibody-dependent cell-mediated cytotoxicity (ADCC) activity. In some embodiments, at least one or two of the heavy chains of the antibody are non-fucosylated. In some embodiments, the antibody is produced in a cell line having a alpha1,6-fucosyltransferase (Fut8) knockout. In some embodiments, the antibody is produced in a cell line overexpressing γ1,4-N-acetylglycosminyltransferase III (GnT-III). In some embodiments, the cell line additionally overexpresses Golgi μ-mannosidase II (ManII). In some embodiments, the antibody comprises at least one amino acid substitution in the Fc region that improves ADCC activity. In some embodiments, the antibody comprises at least one amino acid substitution in the Fc region that improves ADCP activity. In some embodiments, the antibody comprises at least one amino acid substitution in the Fc region that improves CDC activity. In some embodiments, the Fc region comprises the substitution G236A, with numbering according to EU index. In some embodiments, the Fc region comprises the substitution G236A, with numbering according to EU index, and the Fc region is non-fucosylated.

In some embodiments of the antibody:
(a) the antibody comprises a light chain comprising a light chain sequence selected from the group consisting of SEQ ID NOs:236-247 and 1000-1022 and/or a heavy chain comprising a heavy chain sequence and a Q-tag, wherein the heavy chain comprises a sequence selected from the group consisting of SEQ ID NOs:224-235 and 1030-1065;
(b) the antibody comprises a light chain comprising a light chain sequence selected from the group consisting of SEQ ID NOs:236-247 and 1000-1022 and/or a heavy chain comprising a heavy chain sequence selected from the group consisting of SEQ ID NOs:1070-1117;
(c) the antibody comprises a light chain comprising SEQ ID NO:236 and/or a heavy chain comprising SEQ ID NO:224;
(d) the antibody comprises a light chain comprising SEQ ID NO:237 and/or a heavy chain comprising SEQ ID NO:225;
(e) the antibody comprises a light chain comprising SEQ ID NO:238 and/or a heavy chain comprising SEQ ID NO:226;
(f) the antibody comprises a light chain comprising SEQ ID NO:239 and/or a heavy chain comprising SEQ ID NO:227;
(g) the antibody comprises a light chain comprising SEQ ID NO:240 and/or a heavy chain comprising SEQ ID NO:228;
(h) the antibody comprises a light chain comprising SEQ ID NO:241 and/or a heavy chain comprising SEQ ID NO:229;
(i) the antibody comprises a light chain comprising SEQ ID NO:242 and/or a heavy chain comprising SEQ ID NO:230;
(j) the antibody comprises a light chain comprising SEQ ID NO:243 and/or a heavy chain comprising SEQ ID NO:231;
(k) the antibody comprises a light chain comprising SEQ ID NO:244 and/or a heavy chain comprising SEQ ID NO:232;
(l) the antibody comprises a light chain comprising SEQ ID NO:245 and/or a heavy chain comprising SEQ ID NO:233;
(m) the antibody comprises a light chain comprising SEQ ID NO:246 and/or a heavy chain comprising SEQ ID NO:234;
(n) the antibody comprises a light chain comprising SEQ ID NO:247 and/or a heavy chain comprising SEQ ID NO:235;
(o) the antibody comprises a light chain comprising SEQ ID NO:1000 and/or a heavy chain comprising SEQ ID NO:1030;
(p) the antibody comprises a light chain comprising SEQ ID NO:1001 and/or a heavy chain comprising SEQ ID NO:1031;
(q) the antibody comprises a light chain comprising SEQ ID NO:1002 and/or a heavy chain comprising SEQ ID NO:1032;
(r) the antibody comprises a light chain comprising SEQ ID NO:1003 and/or a heavy chain comprising SEQ ID NO:1033;
(s) the antibody comprises a light chain comprising SEQ ID NO:1004 and/or a heavy chain comprising SEQ ID NO:1034;
(t) the antibody comprises a light chain comprising SEQ ID NO:1005 and/or a heavy chain comprising SEQ ID NO:1035;
(u) the antibody comprises a light chain comprising SEQ ID NO:1006 and/or a heavy chain comprising SEQ ID NO:1036;
(v) antibody comprises a light chain comprising SEQ ID NO:1007 and/or a heavy chain comprising SEQ ID NO:1037;
(w) the antibody comprises a light chain comprising SEQ ID NO:1008 and/or a heavy chain comprising SEQ ID NO:1038;
(x) the antibody comprises a light chain comprising SEQ ID NO:1009 and/or a heavy chain comprising SEQ ID NO:1039;
(y) the antibody comprises a light chain comprising SEQ ID NO:1010 and/or a heavy chain comprising SEQ ID NO:1040;
(z) the antibody comprises a light chain comprising SEQ ID NO:1011 and/or a heavy chain comprising SEQ ID NO:1041;
(aa) the antibody comprises a light chain comprising SEQ ID NO:1012 and/or a heavy chain comprising SEQ ID NO:1042;
(bb) the antibody comprises a light chain comprising SEQ ID NO:1013 and/or a heavy chain comprising SEQ ID NO:1043;
(cc) the antibody comprises a light chain comprising SEQ ID NO:1014 and/or a heavy chain comprising SEQ ID NO:1044;
(dd) the antibody comprises a light chain comprising SEQ ID NO:1015 and/or a heavy chain comprising SEQ ID NO:1045;
(ee) the antibody comprises a light chain comprising SEQ ID NO:1016 and/or a heavy chain comprising SEQ ID NO:1046;
(ff) the antibody comprises a light chain comprising SEQ ID NO:1017 and/or a heavy chain comprising SEQ ID NO:1047;
(gg) the antibody comprises a light chain comprising SEQ ID NO:1018 and/or a heavy chain comprising SEQ ID NO:1048;
(hh) the antibody comprises a light chain comprising SEQ ID NO:1019 and/or a heavy chain comprising SEQ ID NO:1049;
(ii) the antibody comprises a light chain comprising SEQ ID NO:1020 and/or a heavy chain comprising SEQ ID NO:1050;
(jj) the antibody comprises a light chain comprising SEQ ID NO:1021 and/or a heavy chain comprising SEQ ID NO:1051;
(kk) the antibody comprises a light chain comprising SEQ ID NO:1022 and/or a heavy chain comprising SEQ ID NO:1052;
(ll) the antibody comprises a light chain comprising SEQ ID NO:236 and/or a heavy chain comprising SEQ ID NO:1053;
(mm) the antibody comprises a light chain comprising SEQ ID NO:242 and/or a heavy chain comprising SEQ ID NO:1054;
(nn) the antibody comprises a light chain comprising SEQ ID NO:243 and/or a heavy chain comprising SEQ ID NO:1055;
(oo) the antibody comprises a light chain comprising SEQ ID NO:245 and/or a heavy chain comprising SEQ ID NO:1056;
(pp) the antibody comprises a light chain comprising SEQ ID NO:1007 and/or a heavy chain comprising SEQ ID NO:1057;
(qq) the antibody comprises a light chain comprising SEQ ID NO:1008 and/or a heavy chain comprising SEQ ID NO:1058;
(rr) the antibody comprises a light chain comprising SEQ ID NO:1012 and/or a heavy chain comprising SEQ ID NO:1059;
(ss) the antibody comprises a light chain comprising SEQ ID NO:1017 and/or a heavy chain comprising SEQ ID NO:1060;
(tt) the antibody comprises a light chain comprising SEQ ID NO:1018 and/or a heavy chain comprising SEQ ID NO:1061;
(uu) the antibody comprises a light chain comprising SEQ ID NO:1019 and/or a heavy chain comprising SEQ ID NO:1062;
(vv) the antibody comprises a light chain comprising SEQ ID NO:1020 and/or a heavy chain comprising SEQ ID NO:1063;
(ww) the antibody comprises a light chain comprising SEQ ID NO:1021 and/or a heavy chain comprising SEQ ID NO:1064;
(xx) the antibody comprises a light chain comprising SEQ ID NO:1022 and/or a heavy chain comprising SEQ ID NO:1065;
(yy) the antibody comprises a light chain comprising SEQ ID NO:236 and/or a heavy chain comprising SEQ ID NO:1070;
(zz) the antibody comprises a light chain comprising SEQ ID NO:237 and/or a heavy chain comprising SEQ ID NO:1071;
(aaa) the antibody comprises a light chain comprising SEQ ID NO:238 and/or a heavy chain comprising SEQ ID NO:1072;
(bbb) the antibody comprises a light chain comprising SEQ ID NO:239 and/or a heavy chain comprising SEQ ID NO:1073;
(ccc) the antibody comprises a light chain comprising SEQ ID NO:240 and/or a heavy chain comprising SEQ ID NO:1074;
(ddd) the antibody comprises a light chain comprising SEQ ID NO:241 and/or a heavy chain comprising SEQ ID NO:1075;
(eee) the antibody comprises a light chain comprising SEQ ID NO:242 and/or a heavy chain comprising SEQ ID NO:1076;
(fff) the antibody comprises a light chain comprising SEQ ID NO:243 and/or a heavy chain comprising SEQ ID NO:1077;
(ggg) the antibody comprises a light chain comprising SEQ ID NO:244 and/or a heavy chain comprising SEQ ID NO:1078;
(hhh) the antibody comprises a light chain comprising SEQ ID NO:245 and/or a heavy chain comprising SEQ ID NO:1079;
(iii) the antibody comprises a light chain comprising SEQ ID NO:246 and/or a heavy chain comprising SEQ ID NO:1080;
(jjj) the antibody comprises a light chain comprising SEQ ID NO:247 and/or a heavy chain comprising SEQ ID NO:1081;
(kkk) the antibody comprises a light chain comprising SEQ ID NO:1000 and/or a heavy chain comprising SEQ ID NO:1082;
(lll) the antibody comprises a light chain comprising SEQ ID NO:1001 and/or a heavy chain comprising SEQ ID NO:1083;
(mmm) the antibody comprises a light chain comprising SEQ ID NO:1002 and/or a heavy chain comprising SEQ ID NO:1084;
(nnn) the antibody comprises a light chain comprising SEQ ID NO:1003 and/or a heavy chain comprising SEQ ID NO:1085;
(ooo) the antibody comprises a light chain comprising SEQ ID NO:1004 and/or a heavy chain comprising SEQ ID NO:1086;
(ppp) the antibody comprises a light chain comprising SEQ ID NO:1005 and/or a heavy chain comprising SEQ ID NO:1087;
(qqq) the antibody comprises a light chain comprising SEQ ID NO:1006 and/or a heavy chain comprising SEQ ID NO:1088;
(rrr) the antibody comprises a light chain comprising SEQ ID NO:1007 and/or a heavy chain comprising SEQ ID NO:1089;
(sss) the antibody comprises a light chain comprising SEQ ID NO:1008 and/or a heavy chain comprising SEQ ID NO:1090;
(ttt) the antibody comprises a light chain comprising SEQ ID NO:1009 and/or a heavy chain comprising SEQ ID NO:1091;
(uuu) the antibody comprises a light chain comprising SEQ ID NO:1010 and/or a heavy chain comprising SEQ ID NO:1092;
(vvv) the antibody comprises a light chain comprising SEQ ID NO:1011 and/or a heavy chain comprising SEQ ID NO:1093;
(www) the antibody comprises a light chain comprising SEQ ID NO:1012 and/or a heavy chain comprising SEQ ID NO:1094;
(xxx) the antibody comprises a light chain comprising SEQ ID NO:1013 and/or a heavy chain comprising SEQ ID NO:1095;
(yyy) the antibody comprises a light chain comprising SEQ ID NO:1014 and/or a heavy chain comprising SEQ ID NO:1096;
(zzz) the antibody comprises a light chain comprising SEQ ID NO:1015 and/or a heavy chain comprising SEQ ID NO:1097;
(aaaa) the antibody comprises a light chain comprising SEQ ID NO:1016 and/or a heavy chain comprising SEQ ID NO:1098;
(bbbb) the antibody comprises a light chain comprising SEQ ID NO:1017 and/or a heavy chain comprising SEQ ID NO:1099;
(cccc) the antibody comprises a light chain comprising SEQ ID NO:1018 and/or a heavy chain comprising SEQ ID NO:1100;
(dddd) the antibody comprises a light chain comprising SEQ ID NO:1019 and/or a heavy chain comprising SEQ ID NO:1101;
(eeee) the antibody comprises a light chain comprising SEQ ID NO:1020 and/or a heavy chain comprising SEQ ID NO:1102;
(ffff) the antibody comprises a light chain comprising SEQ ID NO:1021 and/or a heavy chain comprising SEQ ID NO:1103;
(gggg) the antibody comprises a light chain comprising SEQ ID NO:1022 and/or a heavy chain comprising SEQ ID NO:1104;
(hhhh) the antibody comprises a light chain comprising SEQ ID NO:236 and/or a heavy chain comprising SEQ ID NO:1105;
(iiii) the antibody comprises a light chain comprising SEQ ID NO:242 and/or a heavy chain comprising SEQ ID NO:1106;
(jjjj) the antibody comprises a light chain comprising SEQ ID NO:243 and/or a heavy chain comprising SEQ ID NO:1107;
(kkkk) the antibody comprises a light chain comprising SEQ ID NO:245 and/or a heavy chain comprising SEQ ID NO:1108;
(llll) the antibody comprises a light chain comprising SEQ ID NO:1007 and/or a heavy chain comprising SEQ ID NO:1109;
(mmmm) the antibody comprises a light chain comprising SEQ ID NO:1008 and/or a heavy chain comprising SEQ ID NO:1110;
(nnnn) the antibody comprises a light chain comprising SEQ ID NO:1012 and/or a heavy chain comprising SEQ ID NO:1111;
(oooo) the antibody comprises a light chain comprising SEQ ID NO:1017 and/or a heavy chain comprising SEQ ID NO:1112;
(pppp) the antibody comprises a light chain comprising SEQ ID NO:1018 and/or a heavy chain comprising SEQ ID NO:1113;
(qqqq) the antibody comprises a light chain comprising SEQ ID NO:1019 and/or a heavy chain comprising SEQ ID NO:1114;
(rrrr) the antibody comprises a light chain comprising SEQ ID NO:1020 and/or a heavy chain comprising SEQ ID NO:1115;
(ssss) the antibody comprises a light chain comprising SEQ ID NO:1021 and/or a heavy chain comprising SEQ ID NO:1116; or
(tttt) the antibody comprises a light chain comprising SEQ ID NO:1022 and/or a heavy chain comprising SEQ ID NO:1117.

In some embodiments, the antibody is a bispecific or multispecific antibody that comprises at least a second VL domain and a second VH domain, wherein the second VL domain and second VH domain specifically bind a different target than human nectin-4.

In one aspect, provided herein is a method for preparing a conjugate that comprises (i) an antibody or antigen-binding fragment thereof (Ab) that specifically binds human nectin-4 and (ii) one or more immunomodulating oligonucleotides (P), wherein the antibody or antigen-binding fragment is linked to one or more Q-tag peptides (Q) comprising the amino acid sequence RPQGF (SEQ ID NO:47), and wherein each immunomodulating oligonucleotide is linked to a Q-tag peptide via an amide bond with the glutamine residue of the Q-tag peptide and a linker (L) as shown in formula (A),

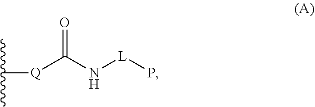

wherein:
[symbol] indicates the point of attachment of each Q to the antibody or antigen-binding fragment thereof (Ab);
each Q independently comprises a Q-tag peptide sequence RPQGF (SEQ ID NO:47);
each L is independently a bond or a linker moiety connected to Q via an amide bond with the glutamine residue; and
each P is independently an immunomodulating oligonucleotide;
   comprising contacting a compound of formula (B)

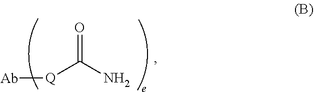

wherein Ab and Q are as defined for formula (A) above, and e is an integer from 1 to 20, with one or more immunomodulating oligonucleotides P, wherein each P independently:
(i) has the following formula:

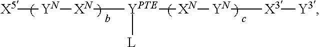

wherein
X^{5′} is a 5′ terminal nucleoside;
X^{Y} is a 3′ terminal nucleoside;
Y^{PTE} is an internucleoside phosphotriester;
Y^{3′} is a terminal phosphotriester;
each X^{N} is independently a nucleoside;
each Y^{N} is independently an internucleoside linker;
b and c are each independently an integer from 1 to 25; with the proviso that the sum of b and c is at least 5; and
L is a linker moiety comprising a terminal amine; or
(ii) is an immunomodulating oligonucleotide of formula (C) or (D);
   in the presence of a transglutaminase. In some embodiments, each immunomodulating oligonucleotide is independently an oligonucleotide of formula (C) or formula (D) is selected from the group consisting of the oligonucleotides of Table 4 and Table 6. In some embodiments, the antibody is according to any one of the antibodies described above. In some embodiments, the method further comprises separating the conjugate having a DAR of 1 from free oligonucleotide, unconjugated antibody, and conjugates having a DAR of 2.

In one aspect, provided herein is a conjugate comprising any one of the antibodies described above conjugated to an agent. In some embodiments, the agent is a label. In some embodiments, the agent is a cytotoxic agent. In some embodiments, the agent is a moiety that modulates the immune system. In some embodiments, the moiety is selected from the group consisting of an IDO/TDO inhibitor, AhR inhibitor, arginase inhibitor, A2aR inhibitor, TLR agonist, STING agonist, and Rig-1 agonist. In some embodiments, the moiety comprises a cytokine. In some embodiments, the cytokine is selected from the group consisting of IL2, IL7, IL10, IL15, or an IFN. In some embodiments, the moiety modulates the activity of a cytokine. In some embodiments, the moiety modulates the activity of IL2, IL7, IL10, IL15, or an interferon. In some embodiments, the moiety comprises a cancer vaccine.

In some embodiments of the antibodies described above or the conjugates described above, the antibody or the antibody of the conjugate is linked to a modified oligonucleotide structure according to any one of the modified oligonucleotide structures of Tables 3-6.

In one aspect, provided herein is a pharmaceutical composition comprising any one of the antibodies described above or any one of the conjugates described above, and a pharmaceutically acceptable carrier.

In one aspect, provided herein is a conjugate comprising (i) an anti-nectin-4 antibody or antigen binding fragment thereof and (ii) and one or more immunomodulating oligonucleotides (P), wherein the antibody or antigen-binding fragment is linked to one or more Q-tag peptides (Q), and wherein each immunomodulating oligonucleotide is linked to a Q-tag peptide via an amide bond with the glutamine residue of the Q-tag peptide and a linker (L) as shown in Formula (A)

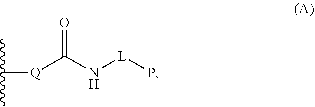

wherein:
[symbol] indicates the point of attachment of each Q to the antibody or antigen-binding fragment thereof (Ab)
each Q independently comprises a Q-tag peptide sequence RPQGFGPP (SEQ ID NO:49);
each L is independently a bond or a linker moiety

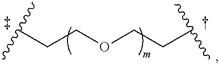

wherein m is an integer ranging from about 0 to about 50, and wherein [symbol] † indicates the point of attachment to P, and [symbol] ‡ indicates the point of attachment to the rest of the conjugate connected to Q via an amide bond with the glutamine residue; and
each P is independently an immunomodulating oligonucleotide having the structure

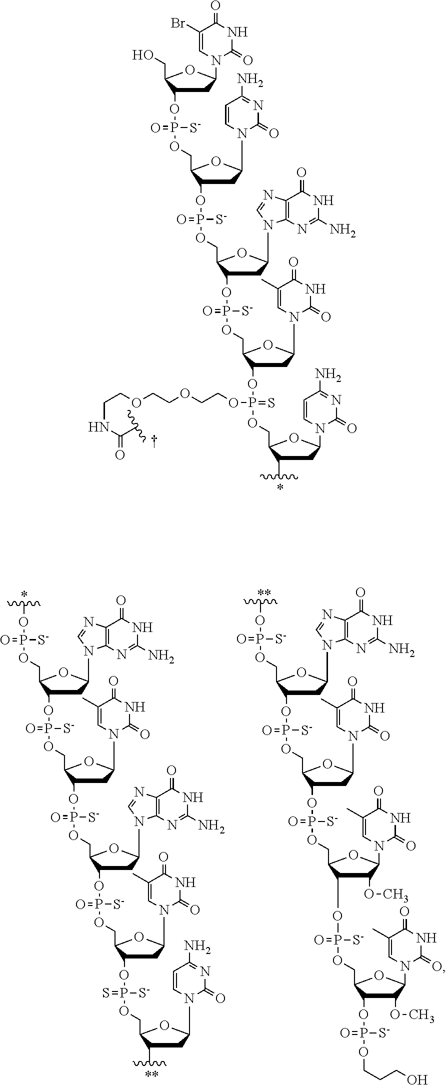

wherein [symbol] and [symbol] ** indicate the points of attachment within the oligonucleotide, and wherein [symbol] † indicates the point of attachment to the linker L;
wherein the antibody comprises a heavy chain variable region (VH) domain and a light chain variable region (VL) domain, wherein the VH domain comprises CDR-H1, CDR-H2, and CDR-H3 and the VL domain comprises CDR-L1, CDR-L2, and CDR-L3, wherein CDR-H1 comprises the sequence of SEQ ID NO:275, CDR-H2 comprises the sequence of SEQ ID NO:276, CDR-H3 comprises the sequence of SEQ ID NO:277, CDR-L1 comprises the sequence of SEQ ID NO:272, CDR-L2 comprises the sequence of SEQ ID NO:273, and CDR-L3 comprises the sequence of SEQ ID NO:274.

In one aspect, provided herein is a conjugate comprising (i) an anti-nectin-4 antibody or antigen binding fragment thereof and (ii) and one or more immunomodulating oligonucleotides (P), wherein the antibody or antigen-binding fragment is linked to one or more Q-tag peptides (Q), and wherein each immunomodulating oligonucleotide is linked to a Q-tag peptide via an amide bond with the glutamine residue of the Q-tag peptide and a linker (L) as shown in Formula (A)

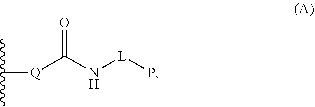

wherein:
[symbol] indicates the point of attachment of each Q to the antibody or antigen-binding fragment thereof (Ab)
each Q independently comprises a Q-tag peptide sequence RPQGFGPP (SEQ ID NO:49);
each L is independently a bond or a linker moiety

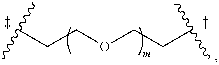

wherein m is an integer ranging from about 0 to about 50, and wherein [symbol] ‡ indicates the point of attachment to P, and [symbol] ‡ indicates the point of attachment to the rest of the conjugate connected to Q via an amide bond with the glutamine residue; and
each P is independently an immunomodulating oligonucleotide having the structure

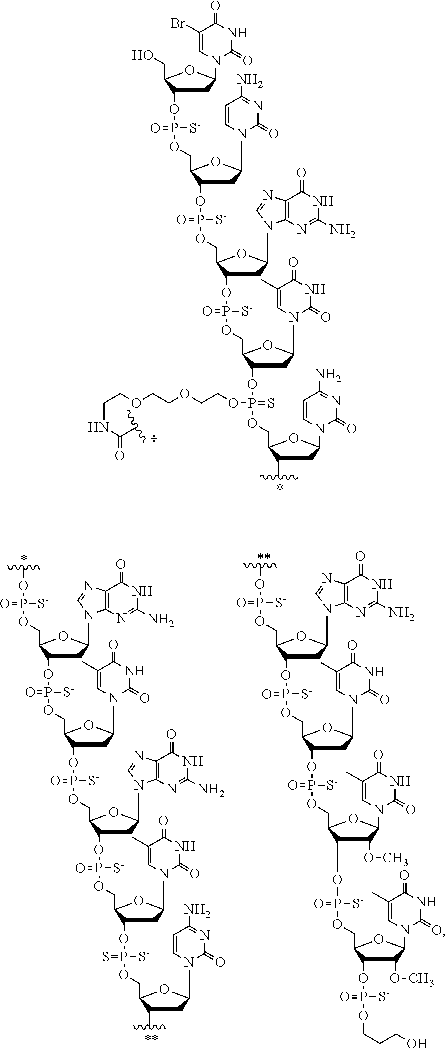

wherein [symbol] * and [symbol] ** indicate the points of attachment within the oligonucleotide, and wherein [symbol] † indicates the point of attachment to the linker L;
wherein the antibody comprises a heavy chain variable region (VH) domain and a light chain variable region (VL) domain, wherein the VH domain comprises CDR-H1, CDR-H2, and CDR-H3 and the VL domain comprises CDR-L1, CDR-L2, and CDR-L3, wherein CDR-H1 comprises the sequence of SEQ ID NO:401, CDR-H2 comprises the sequence of SEQ ID NO:402, CDR-H3 comprises the sequence of SEQ ID NO:403, CDR-L1 comprises the sequence of SEQ ID NO:398, CDR-L2 comprises the sequence of SEQ ID NO:399, and CDR-L3 comprises the sequence of SEQ ID NO:400.

In one aspect, provided herein is a conjugate comprising (i) an anti-nectin-4 antibody or antigen binding fragment thereof and (ii) and one or more immunomodulating oligonucleotides (P), wherein the antibody or antigen-binding fragment is linked to one or more Q-tag peptides (Q), and wherein each immunomodulating oligonucleotide is linked to a Q-tag peptide via an amide bond with the glutamine residue of the Q-tag peptide and a linker (L) as shown in Formula (A)

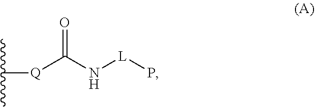

wherein:
[symbol] indicates the point of attachment of each Q to the antibody or antigen-binding fragment thereof (Ab)
each Q independently comprises a Q-tag peptide sequence RPQGFGPP (SEQ ID NO:49);
each L is independently a bond or a linker moiety

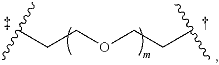

wherein m is an integer ranging from about 0 to about 50, and wherein [symbol] † indicates the point of attachment to P, and [symbol] ‡ indicates the point of attachment to the rest of the conjugate connected to Q via an amide bond with the glutamine residue; and
each P is independently an immunomodulating oligonucleotide having the structure

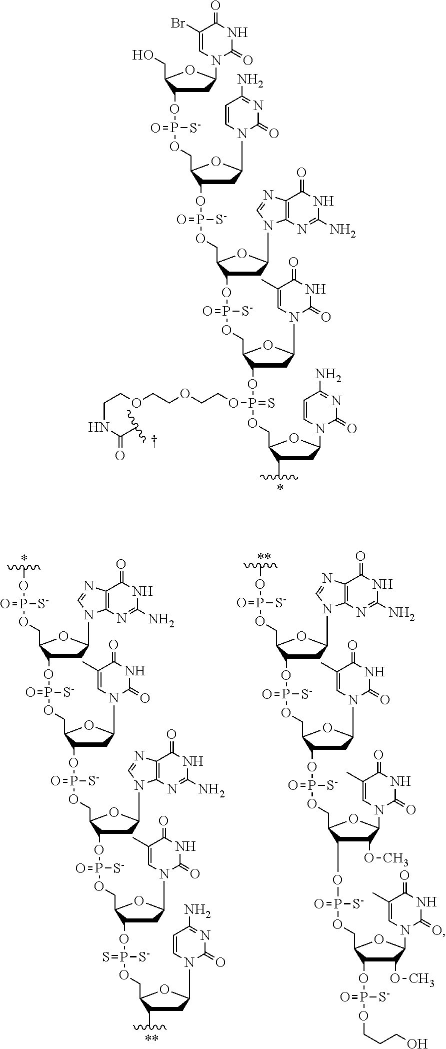

wherein [symbol] * and [symbol] ** indicate the points of attachment within the oligonucleotide, and wherein [symbol] † indicates the point of attachment to the linker L;
wherein the antibody comprises a heavy chain variable region (VH) domain and a light chain variable region (VL) domain, wherein the VH domain comprises CDR-H1, CDR-H2, and CDR-H3 and the VL domain comprises CDR-L1, CDR-L2, and CDR-L3, wherein CDR-H1 comprises the sequence of SEQ ID NO:679, CDR-H2 comprises the sequence of SEQ ID NO:680, CDR-H3 comprises the sequence of SEQ ID NO:681, CDR-L1 comprises the sequence of SEQ ID NO:676, CDR-L2 comprises the sequence of SEQ ID NO:677, and CDR-L3 comprises the sequence of SEQ ID NO:678.

In one aspect, provided herein is a conjugate comprising (i) an anti-nectin-4 antibody or antigen binding fragment thereof and (ii) and one or more immunomodulating oligonucleotides (P), wherein the antibody or antigen-binding fragment is linked to one or more Q-tag peptides (Q), and wherein each immunomodulating oligonucleotide is linked to a Q-tag peptide via an amide bond with the glutamine residue of the Q-tag peptide and a linker (L) as shown in Formula (A)

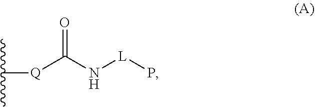

wherein:
[symbol] indicates the point of attachment of each Q to the antibody or antigen-binding fragment thereof (Ab)
each Q independently comprises a Q-tag peptide sequence RPQGFGPP (SEQ ID NO:49);
each L is independently a bond or a linker moiety

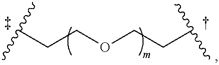

wherein m is an integer ranging from about 0 to about 50, and wherein [symbol] † indicates the point of attachment to P, and [symbol] ‡ indicates the point of attachment to the rest of the conjugate connected to Q via an amide bond with the glutamine residue; and
each P is independently an immunomodulating oligonucleotide having the structure

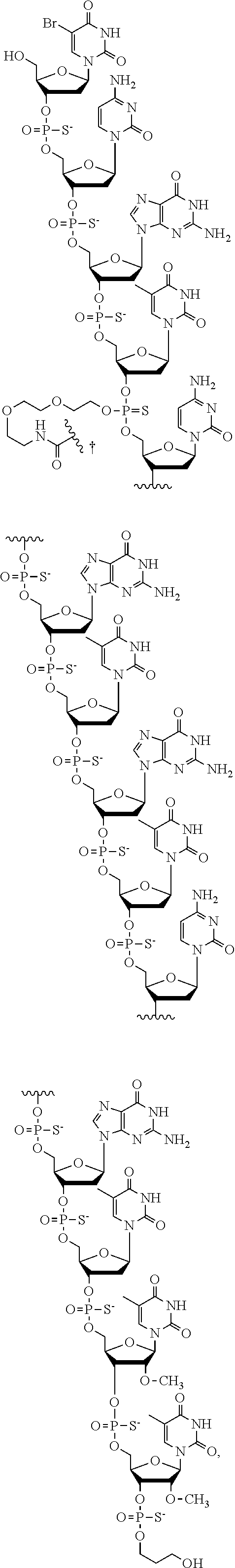

wherein [symbol] * and [symbol] ** indicate the points of attachment within the oligonucleotide, and wherein [symbol] † indicates the point of attachment to the linker L;
wherein the antibody comprises a heavy chain variable region (VH) domain and a light chain variable region (VL) domain, wherein the VH domain comprises CDR-H1, CDR-H2, and CDR-H3 and the VL domain comprises CDR-L1, CDR-L2, and CDR-L3, wherein CDR-H1 comprises the sequence of SEQ ID NO:275, CDR-H2 comprises the sequence of SEQ ID NO:276, CDR-H3 comprises the sequence of SEQ ID NO:277, CDR-L1 comprises the sequence of SEQ ID NO:272, CDR-L2 comprises the sequence of SEQ ID NO:273, and CDR-L3 comprises the sequence of SEQ ID NO:274.

In one aspect, provided herein is a conjugate comprising (i) an anti-nectin-4 antibody or antigen binding fragment thereof and (ii) and one or more immunomodulating oligonucleotides (P), wherein the antibody or antigen-binding fragment is linked to one or more Q-tag peptides (Q), and wherein each immunomodulating oligonucleotide is linked to a Q-tag peptide via an amide bond with the glutamine residue of the Q-tag peptide and a linker (L) as shown in Formula (A)

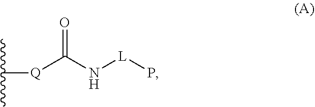

wherein:
[symbol] indicates the point of attachment of each Q to the antibody or antigen-binding fragment thereof (Ab)
each Q independently comprises a Q-tag peptide sequence RPQGFGPP (SEQ ID NO:49);
each L is independently a bond or a linker moiety

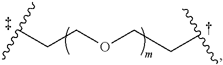

wherein m is an integer ranging from about 0 to about 50, and wherein [symbol] † indicates the point of attachment to P, and [symbol] ‡ indicates the point of attachment to the rest of the conjugate connected to Q via an amide bond with the glutamine residue; and
each P is independently an immunomodulating oligonucleotide having the structure

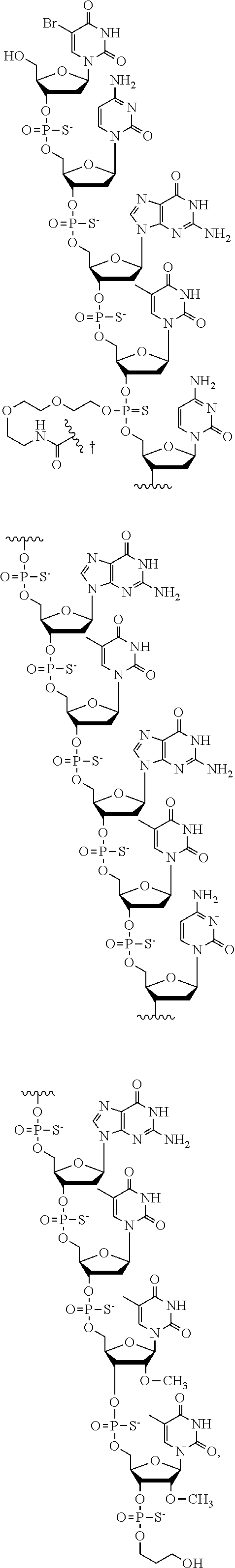

wherein [symbol] * and [symbol] ** indicate the points of attachment within the oligonucleotide, and wherein [symbol] † indicates the point of attachment to the linker L;
   wherein the antibody comprises a heavy chain variable region (VH) domain and a light chain variable region (VL) domain, wherein the VH domain comprises CDR-H1, CDR-H2, and CDR-H3 and the VL domain comprises CDR-L1, CDR-L2, and CDR-L3, wherein CDR-H1 comprises the sequence of SEQ ID NO:401, CDR-H2 comprises the sequence of SEQ ID NO:402, CDR-H3 comprises the sequence of SEQ ID NO:403, CDR-L1 comprises the sequence of SEQ ID NO:398, CDR-L2 comprises the sequence of SEQ ID NO:399, and CDR-L3 comprises the sequence of SEQ ID NO:400.

In one aspect, provided herein is a conjugate comprising (i) an anti-nectin-4 antibody or antigen binding fragment thereof and (ii) and one or more immunomodulating oligonucleotides (P), wherein the antibody or antigen-binding fragment is linked to one or more Q-tag peptides (Q), and wherein each immunomodulating oligonucleotide is linked to a Q-tag peptide via an amide bond with the glutamine residue of the Q-tag peptide and a linker (L) as shown in Formula (A)

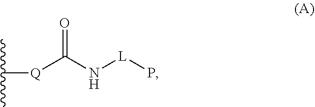

wherein:
[symbol] indicates the point of attachment of each Q to the antibody or antigen-binding fragment thereof (Ab)
each Q independently comprises a Q-tag peptide sequence RPQGFGPP (SEQ ID NO:49);
each L is independently a bond or a linker moiety

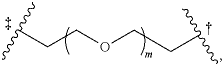

wherein m is an integer ranging from about 0 to about 50, and wherein [symbol] † indicates the point of attachment to P, and [symbol] indicates the point of attachment to the rest of the conjugate connected to Q via an amide bond with the glutamine residue; and
each P is independently an immunomodulating oligonucleotide having the structure

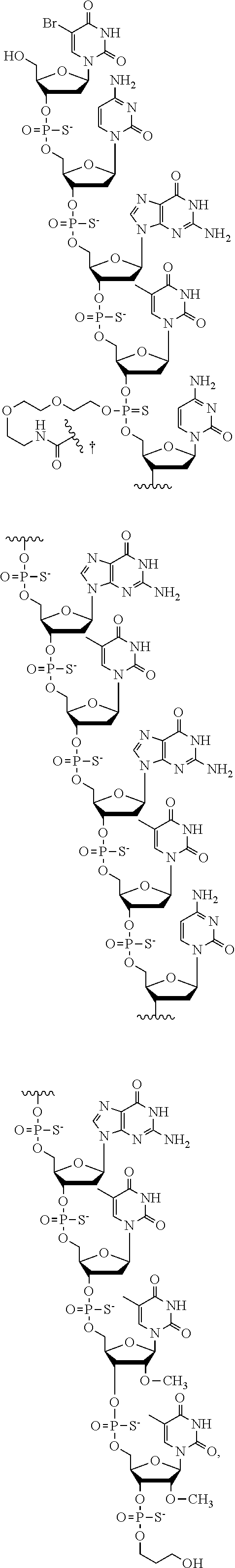

wherein [symbol] * and [symbol] ** indicate the points of attachment within the oligonucleotide, and wherein [symbol] † indicates the point of attachment to the linker L;
   wherein the antibody comprises a heavy chain variable region (VH) domain and a light chain variable region (VL) domain, wherein the VH domain comprises CDR-H1, CDR-H2, and CDR-H3 and the VL domain comprises CDR-L1, CDR-L2, and CDR-L3, wherein CDR-H1 comprises the sequence of SEQ ID NO:679, CDR-H2 comprises the sequence of SEQ ID NO:680, CDR-H3 comprises the sequence of SEQ ID NO:681, CDR-L1 comprises the sequence of SEQ ID NO:676, CDR-L2 comprises the sequence of SEQ ID NO:677, and CDR-L3 comprises the sequence of SEQ ID NO:678.

In some embodiments of the conjugate, the antibody of the conjugate comprises (a) a heavy chain variable region (VH) domain comprising SEQ ID NO: 935 and a light chain variable region (VL) domain comprising SEQ ID NO: 934; (b) a heavy chain comprising a heavy chain sequence and a Q-tag, wherein the heavy chain comprises SEQ ID NO: 1047, and a light chain comprising SEQ ID NO: 1017; (c) a heavy chain comprising a heavy chain sequence and a Q-tag, wherein the heavy chain comprises SEQ ID NO: 1060 and a light chain comprising SEQ ID NO: 1017; (d) a heavy chain comprising SEQ ID NO:1099 and a light chain comprising SEQ ID NO:1017; or (e) a heavy chain comprising SEQ ID NO:1112 and a light chain comprising SEQ ID NO:1017. In some embodiments of the conjugate, the antibody of the conjugate comprises (a) a heavy chain variable region (VH) domain comprising SEQ ID NO: 937 and a light chain variable region (VL) domain comprising SEQ ID NO: 936; (b) a heavy chain comprising a heavy chain sequence and a Q-tag, wherein the heavy chain comprises SEQ ID NO: 1048, and a light chain comprising SEQ ID NO:1018; (c) a heavy chain comprising a heavy chain sequence and a Q-tag, wherein the heavy chain comprises SEQ ID NO: 1061 and a light chain comprising SEQ ID NO:1018; (d) a heavy chain comprising SEQ ID NO:1100 and a light chain comprising SEQ ID NO:1018; or (e) a heavy chain comprising SEQ ID NO:1113 and a light chain comprising SEQ ID NO:1018. In some embodiments of the conjugate, the antibody of the conjugate comprises (a) a heavy chain variable region (VH) domain comprising SEQ ID NO: 939 and a light chain variable region (VL) domain comprising SEQ ID NO: 938; (b) a heavy chain comprising a heavy chain sequence and a Q-tag, wherein the heavy chain comprises SEQ ID NO: 1049, and a light chain comprising SEQ ID NO: 1019; (c) a heavy chain comprising a heavy chain sequence and a Q-tag, wherein the heavy chain comprises SEQ ID NO: 1062 and a light chain comprising SEQ ID NO: 1019; (d) a heavy chain comprising SEQ ID NO:1101 and a light chain comprising SEQ ID NO:1019; or (e) a heavy chain comprising SEQ ID NO:1114 and a light chain comprising SEQ ID NO:1019.

In some embodiments of the conjugate, m is from about 20 to about 30. In some embodiments of the conjugate, m is about 24 or m is 24. In some embodiments of the conjugate, L comprises a polyethylene glycol moiety. In some embodiments of the conjugate, the polyethylene glycol moiety contains about 24 ethylene glycol units or contains 24 ethylene glycol units. In some embodiments of the conjugate, each (P) and (L) in the conjugate independently comprise a modified oligonucleotide of SEQ ID NO:35. In some embodiments of the conjugate, each (P) and (L) in the conjugate independently comprise a modified oligonucleotide of SEQ ID NO:9. In some embodiments of the conjugate, the antibody of the conjugate comprises an Fc region. In some embodiments of the conjugate, the Fc region is a human IgG1 Fc region. In some embodiments of the conjugate, the Fc region is non-fucosylated. In some embodiments of the conjugate, the Fc region comprises the substitution G236A, with numbering according to EU index. In some embodiments of the conjugate, the Fc region comprises the substitution G236A, with numbering according to EU index, and the Fc region is non-fucosylated. In some embodiments of the conjugate, the conjugate has a DAR of 1 or 2.

In one aspect, provided herein is a pharmaceutical composition comprising any one of the conjugates described above and a pharmaceutically acceptable carrier.

In one aspect, provided herein is a method for treating cancer, comprising administering to an individual an effective amount of any one of the conjugates described above, any one of the antibodies described above, or any one of the pharmaceutical compositions described above. In some embodiments, the cancer tumor cells overexpress nectin-4 compared to the normal cells the cancer is derived from. In some embodiments, the cancer tumor cells express normal or moderate levels of nectin-4 compared to normal cells the cancer is derived from. In some embodiments, the cancer tumor cells express low levels of nectin-4 compared to the normal cells the cancer is derived from. In some embodiments, the method further comprises administering at least one additional cancer therapeutic. In some embodiments, at least one additional cancer therapeutic comprises a chemotherapeutic, an immunotherapeutic, a small molecule inhibitor (SMQ), a therapeutic antibody, or a cancer vaccine. In some embodiments, the cancer is a solid cancer. In some embodiments, the cancer is a liquid tumor. In some embodiments, the cancer is esophageal cancer, stomach cancer, breast cancer, ovarian cancer, lung cancer, pancreatic adenocarcinoma, colon carcinoma, bladder cancer, cervical cancer, thyroid cancer, uterine cancer, rectal cancer, or gallbladder cancer. In some embodiments, administration of the conjugate or the pharmaceutical composition activates T cells, dendritic cells, monocytes, and/or NK cells.

In one aspect, provided herein is a method of activating immune cells in an individual, the method comprising administering to the individual an effective amount of any one of the conjugates described above or any one of the pharmaceutical compositions described above comprising a conjugate, wherein administration of the conjugate or the pharmaceutical composition activates T cells, dendritic cells, monocytes, and/or NK cells. In some embodiments, the individual has a cancer. In some embodiments, the method further comprises administering at least one additional cancer therapeutic. In some embodiments, the at least one additional cancer therapeutic comprises a chemotherapeutic, an immunotherapeutic, a small molecule inhibitor (SMQ), a therapeutic antibody, or a cancer vaccine. In some embodiments, the cancer is a solid cancer. In some embodiments, the cancer is a liquid tumor. In some embodiments, the cancer is esophageal cancer, stomach cancer, breast cancer, ovarian cancer, lung cancer, pancreatic adenocarcinoma, colon carcinoma, bladder cancer, cervical cancer, thyroid cancer, uterine cancer, rectal cancer, or gallbladder cancer. In some embodiments, the cancer is refractory to immune checkpoint inhibitor therapy. In some embodiments, the immune checkpoint inhibitor therapy comprises a PD-1 inhibitor or a PD-L1 inhibitor.

In one aspect, provided herein is a method of treating a tumor or tumor cells, comprising administering an effective amount of any one of the conjugates described above, any one of the antibodies described above, or any one of the pharmaceutical compositions described above. In some embodiments, treating the tumor or tumor cells comprises killing the tumor or tumor cells, inducing cytotoxicity of the tumor or tumor cells, reducing or inhibiting viability of the tumor or tumor cells, inhibiting growth of the tumor or tumor cells, inhibiting establishment of the tumor or tumor cells, inhibiting or reducing metastasis of the tumor or tumor cells, reducing the size of the tumor, or activiating an immune response against the tumor or tumor cells.

In one aspect, provided herein is a nucleic acid encoding the antibody of any one of the conjugate described above or any one of the antibodies described above.

In one aspect, provided herein is a vector comprising the nucleic acid described above. In some embodiments, the vector is an expression vector.

In one aspect, provided herein is a host cell comprising any one of the vectors described above. In some embodiments, the host cell is a mammalian cell, a yeast cell, or a bacterial cell. In some embodiments, the host cell is a CHO cell.

In one aspect, provided herein is a method of producing any one of the antibodies described above, the method comprising culturing any one of the host cells described above under conditions to express the antibody in the host cell. In some embodiments, the method further comprises isolating and/or purifying the antibody from the host cell.

### BRIEF DESCRIPTION OF THE FIGURES

The present application can be understood by reference to the following description taken in conjunction with the accompanying figures.

FIGS. 1A-1B depict the activity of immunomodulating oligonucleotides alone in human PBMCs based upon observed increased expression of (a) HLADR (FIG. 1A) and (b) CD40 (FIG. 1B).

FIGS. 2A-2C show the effect of immunomodulating oligonucleotides and their antibody conjugates on increasing B cells numbers and activation. FIG. 2A depicts the observed effect on B cell numbers by the various immunomodulating polypeptides alone. FIGS. 2B-2C depict the observed activation of B cells (via detection of CD40 expression) produced by the immunomodulating oligonucleotides alone.

FIG. 3 shows the percentage yields of transglutaminase-mediated conjugations and deconjugations with a polyethylene glycol linker (—NH—C(═O)—PEG₂₃-NH₂) and various Q-tags. Shown are SEQ ID Nos:39-47 and 50-52.

FIGS. 4A and 4B show the percentage change of conjugation and decongjuation over time in transglutaminase conjugation of two Q-tag peptides (LSLSPGLLQGG, SEQ ID NO:39; and RPQGF, SEQ ID NO:47).

FIG. 5 shows activity of indicated free CpG oligonucleotides on human PBMCs, as assayed by CD40 expression on CD19+B cells.

FIG. 6 shows activity of indicated free CpG oligonucleotides on human PBMCs, as assayed by Ramos NFkb Reporter Assay.

FIGS. 7A-7C show activity of indicated free CpG oligonucleotides on human PBMCs from three different donor lines (D559, FIG. 7A, D804, FIG. 7B, and D643, FIG. 7C), as observed by CD40 expression.

FIG. 8 shows activity of indicated free CpG oligonucleotides on human PBMCs, as assayed by CD40 expression on CD19+B cells.

FIGS. 9A-9D show a schematic diagram of exemplary conjugates, in accordance with some embodiments. Exemplary antibody:CpG conjugates with an engineered Q-tag fused to the C-terminus of the heavy chain are shown in FIG. 9A (with a DAR 1) and in FIG. 9B (with DAR 2). Exemplary antibody:CpG conjugates with a naturally occurring Q-tag (Q295) exposed for conjugation by an N297A mutation are shown in FIG. 9C (with a DAR 1) and in FIG. 9D (with DAR 2).

FIG. 10 shows the results of a competition experiment between various nectin-4 antibodies.

FIG. 11 shows monocyte activation by nectin-4 antibody-CpG conjugates in nectin-4-expressing cells (left) and nectin-4 negative cells (right).

FIG. 12 shows dendritic cell activation by nectin-4 antibody-CpG conjugates in nectin-4-expressing cells (left) and nectin-4 negative cells (right).

FIG. 13 shows enhancement of tumor phagocytosis by various nectin-4-antibody-conjugates.

FIGS. 14A-14D shows enhancement of cytokine secretion from PBMCs by various nectin-4 antibody-CpG conjugates graphed as fold activity over the corresponding unconjugated nectin-4 antibody. FIG. 14A shows IFNα2 induction, FIG. 14B shows IFNγ induction, FIG. 14C shows IL6 induction, and FIG. 14D shows IL10 induction.

FIG. 15 shows internalization of nectin-4 antibodies.

FIGS. 16A-16B shows anti-tumor activity of a nectin-4 antibody conjugate on MC38 nectin-4 tumors. FIG. 16A shows the broad expression of nectin-4 on MC38 nectin-4 cells and FIG. 16B shows anti-tumor activity of a nectin-4 antibody conjugate.

FIG. 17 shows activation of T cells by nectin-4 antibody conjugates, as assayed by CD69 percentage on CD3⁺ T cells.

FIG. 18 shows activation of NK cells by nectin-4 antibody conjugates, as assayed by CD69 expression on CD56⁺CD3⁻NK cells.

FIG. 19 shows activation of NK cells by nectin-4 antibody conjugates, as assayed by IRF7 expression.

FIG. 20A-20B shows in vivo anti-tumor activity of nectin-4 antibody conjugates. The arrows show days of administration.

FIG. 21 shows binding of various nectin-4 antibodies to SKBR3 cells endogenously expressing nectin-4.

FIG. 22 shows internalization of indicated nectin-4 antibodies in T47D (top) or nectin-4-DLD cells (bottom).

FIG. 23 shows immune cell activation by nectin-4 antibody conjugates in nectin-4-DLD cells graphed as fold over the corresponding unconjugated nectin-4 antibody.

FIG. 24 shows immune cell activation by nectin-4 antibody conjugates in OE19 cells graphed as fold over the corresponding unconjugated nectin-4 antibody.

FIG. 25 shows immune cell activation by nectin-4 antibody conjugates in nectin-4-DLD cells graphed as fold over the corresponding unconjugated nectin-4 antibody.

FIG. 26 shows immune cell activation by nectin-4 antibody conjugates in HT1376 cells graphed as fold over the corresponding unconjugated nectin-4 antibody.

FIG. 27 shows immune cell activation by nectin-4 antibody conjugates in H292 cells graphed as fold over the corresponding unconjugated nectin-4 antibody.

FIG. 28 shows immune cell activation in OE19 cells by a nectin-4 antibody CpG conjugate (TNT-266a, left) or a nectin-4 antibody conjugated to a TLR8 agonist (the conjugate is designated TNT-347xx, right) graphed as fold over the corresponding unconjugated nectin-4 antibody.

FIG. 29 shows anti-tumor activity of a nectin-4 antibody conjugate and nectin-4 antibody MMAE conjugate on MC38 nectin-4 tumors.

FIG. 30 shows immune cell activation by nectin-4 antibody conjugates with human IgG1 or non-fucosylated human IgG1 in nectin-4 DLD cells.

FIG. 31 shows B cell activation by nectin-4 antibody conjugates in nectin-4 DLD (top left), HT1376 (top right), OE19 (bottom left), and H292 (bottom right) cells.

FIG. 32 shows B cell activation by nectin-4 antibody conjugates in nectin-4 DLD (top left), HT1376 (middle right), and H292 (bottom left) cells.

FIG. 33 shows memory response in MC38 tumor cells after treatment with the indicated antibody conjugates or PBS.

FIG. 34 shows in vivo anti-tumor activity of nectin-4 antibody conjugates against SKBR3 tumors.

FIGS. 35A-35B show in vivo anti-tumor activity of nectin-4 antibody conjugates against nectin-4 expressing CT26 tumors (FIG. 35A) and EMT6 tumors (FIG. 35B).

FIGS. 36A-36B shows in vivo anti-tumor activity of nectin-4 antibody conjugates against Nectin-4 expressing CT26 tumors (FIG. 36A) and EMT6 tumors (FIG. 36B).

FIG. 37 a comparison of in vivo anti-tumor activity of nectin-4 antibody conjugates with DAR1 and DAR2.

FIG. 38 shows the activity of fucosylated and non-fucosylated conjugates in HT1376 and H292 cell lines.

FIG. 39 shows the activity of fucosylated and non-fucosylated nectin-4 antibody conjugates on dendritic cell proliferation.

FIG. 40 shows the activity of nectin-4 antibody conjugates on tumor cell cytotoxicity.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

The present disclosure is based, at least in part, on the discovery of novel nectin-4 antibodies (i.e., anti-nectin-4 antibodies; antibodies that bind nectin-4; antibodies that specifically bind nectin-4). The disclosure is further based, in part, on the conjugation of nectin-4 antibodies to an agent, such as an immunomodulating oligonucleotide. In some embodiments, the nectin-4 antibodies are conjugated to CpG oligonucleotides (i.e., also referred to herein as nectin-4 antibody CpG conjugates, nectin-4 antibody-oligonucleotide conjugates, nectin-4 antibody-conjugates, anti-nectin-4 antibody conjugates, or, unless context indicates otherwise, a “conjugate”) which provide targeted delivery of the CpG oligonucleotides for TLR9 activation. Means of preparing these conjugates are also described. Particularly, the conjugation can be performed by a transglutaminase (Tgase)-mediated reaction. Also provided are intermediate compounds which can be used to prepare these conjugates.

Also provided herein are pharmaceutical compositions comprising the nectin-4 antibody conjugates and nectin-4 antibodies, kits, and methods of treatment (including methods of treating cancer) using the conjugates and antibodies.

### I. Definitions

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to particular method steps, reagents, or conditions are specifically embraced by the present disclosure and are disclosed herein just as if each and every combination was individually and explicitly disclosed.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Throughout this application, unless the context indicates otherwise, references to a compound of Formula (A)-(D) include ionic forms, polymorphs, pseudopolymorphs, amorphous forms, solvates, co-crystals, chelates, isomers, tautomers, oxides (e.g., N-oxides, S⁻oxides), esters, prodrugs, isotopes and/or protected forms thereof. In some embodiments, references to a compound of Formula (A)-(D) include polymorphs, solvates, co-crystals, isomers, tautomers and/or oxides thereof. In some embodiments, references to a compound of Formula (A)-(D) include polymorphs, solvates, and/or co-crystals thereof. In some embodiments, references to a compound of Formula (A)-(D) include isomers, tautomers and/or oxides thereof. In some embodiments, references to a compound of Formula (A)-(D) include solvates thereof.

“Alkyl” encompasses straight and branched carbon chains having the indicated number of carbon atoms, for example, from 1 to 20 carbon atoms, or 1 to 8 carbon atoms, or 1 to 6 carbon atoms. For example, C₁-6 alkyl encompasses both straight and branched chain alkyl of from 1 to 6 carbon atoms. When an alkyl residue having a specific number of carbons is named, all branched and straight chain versions having that number of carbons are intended to be encompassed; thus, for example, “propyl” includes n-propyl and isopropyl; and “butyl” includes n-butyl, sec-butyl, isobutyl and t-butyl. Examples of alkyl groups include, but are not limited to, methyl, ethyl, propyl, isopropyl, n-butyl, sec-butyl, tert-butyl, pentyl, 2-pentyl, 3-pentyl, isopentyl, neopentyl, hexyl, 2-hexyl, 3-hexyl, and 3-methylpentyl.

When a range of values is given (e.g., C₁₋₆ alkyl), each value within the range as well as all intervening ranges are included. For example, “C₁₋₆ alkyl” includes C₁, C₂, C₃, C₄, C₅, C₆, C₁₋₆, C₂₋₆, C₃₋₆, C₄₋₆, C₅₋₆, C₁₋₅, C₂₋₅, C₃₋₅, C₄₋₅, C₁₋₄, C₂₋₄, C₃₋₄, C₁₋₃, C₂₋₃, and C₁₋₂ alkyl.

“Alkenyl” refers to an unsaturated branched or straight-chain alkyl group having the indicated number of carbon atoms (e.g., 2 to 8, or 2 to 6 carbon atoms) and at least one carbon-carbon double bond. The group may be in either the cis or trans configuration (Z or E configuration) about the double bond(s). Alkenyl groups include, but are not limited to, ethenyl, propenyl (e.g., prop-1-en-1-yl, prop-1-en-2-yl, prop-2-en-1-yl (allyl), prop-2-en-2-yl), and butenyl (e.g., but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl).

“Alkynyl” refers to an unsaturated branched or straight-chain alkyl group having the indicated number of carbon atoms (e.g., 2 to 8 or 2 to 6 carbon atoms) and at least one carbon-carbon triple bond. Alkynyl groups include, but are not limited to, ethynyl, propynyl (e.g., prop-1-yn-1-yl, prop-2-yn-1-yl) and butynyl (e.g., but-1-yn-1-yl, but-1-yn-3-yl, but-3-yn-1-yl).

The term “amino,” as used herein, represents —N(R^{N1})₂, where, if amino is unsubstituted, both R^{N1} are H; or, if amino is substituted, each R^{N1} is independently H, —OH, —NO₂, —N(R^{N2})₂, —SO₂OR^{N2}, —SO₂R^{N2}, —SOR^{N2}, —COOR^{N2}, an N-protecting group, alkyl, alkenyl, alkynyl, alkoxy, aryl, arylalkyl, aryloxy, cycloalkyl, cycloalkenyl, heteroalkyl, or heterocyclyl, provided that at least one R^{N1} is not H, and where each R^{N2} is independently H, alkyl, or aryl. Each of the substituents may itself be unsubstituted or substituted with unsubstituted substituent(s) defined herein for each respective group. In some embodiments, amino is unsubstituted amino (i.e., —NH₂) or substituted amino (e.g., —NHR^{N1}), where R^{N1} is independently —OH, —SO₂OR^{N2}, SO₂R^{N2}, —SOR^{N2}, —COOR^{N2}, optionally substituted alkyl, or optionally substituted aryl, and each R^{N2} can be optionally substituted alkyl or optionally substituted aryl. In some embodiments, substituted amino may be alkylamino, in which the alkyl groups are optionally substituted as described herein for alkyl. In certain embodiments, an amino group is —NHR^{N1}, in which R^{N1} is optionally substituted alkyl. Non-limiting examples of —NHR^{N1}, in which R^{N1} is optionally substituted alkyl, include: optionally substituted alkylamino, a proteinogenic amino acid, a non-proteinogenic amino acid, a C₁₋₆ alkyl ester of a proteinogenic amino acid, and a C₁₋₆ alkyl ester of a non-proteinogenic amino acid. The amino acid employed is optionally in the L-form.

The term “immunomodulating oligonucleotide” as used herein, represents a oligonucleotide construct containing a total of from 6 to 50 contiguous nucleosides covalently bound together by internucleoside bridging groups independently selected from the group consisting of internucleoside phosphoesters and optionally internucleoside abasic spacers. The immunomodulating oligonucleotides are capped at 5′- and 3′-termini with 5′- and 3′-capping groups, respectively. The immunomodulating oligonucleotides are capable of modulating an innate immune response, as determined by, e.g., a change in the activation of intracellular signaling pathway(s) including but not limited to NFκB, a change in the expression of an activation marker or a change in the secretion of at least one inflammatory cytokine or at least one type I interferon in an immune cell (e.g., antigen-presenting cell) to which an immunomodulating oligonucleotide was delivered (e.g., in comparison to another immune cell (e.g., antigen-presenting cell) to which an immunomodulating oligonucleotide was not delivered) or in an immune cell that interacts with an immune cell (e.g., antigen-presenting cell) to which an immunomodulating oligonucleotide was delivered (including direct cell-to-cell interactions as well as indirect stimulation, e.g., from one or more cytokines secreted by the cell to which an immunomodulating oligonucleotide was delivered). The immunomodulating oligonucleotide may contain a conjugating group or, if the immunomodulating oligonucleotide is part of a conjugate, a linker bonded to a targeting moiety and optionally to one or more (e.g., 1 to 6) auxiliary moieties (e.g., polyethylene glycols). The conjugating group or the linker may be part of the phosphotriester or the terminal capping group.

The term “immunostimulating oligonucleotide” as used herein, represents an immunomodulating oligonucleotide capable of activating an immune response, as determined by, e.g., an increase in the activation of intracellular signaling pathway(s) such as NFκB or an increase in levels of cell surface marker(s) of activation or function or an increase in the secretion of at least one inflammatory cytokine or at least one type I interferon in an immune cell (e.g., antigen-presenting cell) to which an immunostimulating oligonucleotide was delivered (e.g., in comparison to another immune cell (e.g., antigen-presenting cell) to which an immunostimulating oligonucleotide was not delivered) or in an immune cell that interacts with an immune cell (e.g., antigen-presenting cell) to which an immunomodulating oligonucleotide was delivered (including direct cell-to-cell interactions as well as indirect stimulation, e.g., from one or more cytokines secreted by the cell to which an immunomodulating oligonucleotide was delivered). In some embodiments, the immunostimulating oligonucleotide contains at least one cytidine-p-guanosine (CpG) sequence, in which p is an internucleoside phosphodiester (e.g., phosphate or phosphorothioate) or an internucleoside phosphotriester or phosphothiotriester. As used herein, the CpG-containing immunostimulating oligonucleotide can be naturally existing, such as CpG ODNs of bacterial or viral origins, or synthetic. For example, in some embodiments, the CpG sequence in the immunostimulating oligonucleotide contains 2′-deoxyribose.

The term “immunosuppressive oligonucleotide” as used herein, represents an immunomodulating oligonucleotide capable of antagonizing an immune response, as determined by e.g., a reduction in the activation or lack of activation of NFκB or lack on increase in the levels of cell surface marker(s) of activation of function or a reduction or lack of increase in the secretion of at least one inflammatory cytokine or at least one type I interferon in an immune cell (e.g., antigen-presenting cell) to which an immunosuppressive oligonucleotide was delivered (e.g., in comparison to another immune cell (e.g., antigen-presenting cell) to which an immunosuppressive oligonucleotide was not delivered) or in an immune cell that interacts with an immune cell (e.g., antigen-presenting cell) to which an immunomodulating oligonucleotide was delivered (including direct cell-to-cell interactions as well as indirect stimulation, e.g., from one or more cytokines secreted by the cell to which an immunomodulating oligonucleotide was delivered).

It is to be understood that the terms “oligonucleotide” and “oligonucleotide” may be used interchangeably herein. It is further to be understood that the terms “immunomodulating oligonucleotide,” “immunostimulating oligonucleotide,” “immunosuppressive oligonucleotide,” and “conjugate” encompass salts of the immunomodulating oligonucleotide, immunostimulating oligonucleotide, immunosuppressive oligonucleotide and conjugate, respectively. For example, the terms “immunomodulating oligonucleotide,” “immunostimulating oligonucleotide,” “immunosuppressive oligonucleotide,” and “conjugate” encompasses both the protonated, neutral form (P-XH moiety, where X is O or S) of a phosphate, phosphorothioate, or phosphorodithioate and the deprotonated, ionic form (P-X⁻moiety, where X is O or S) of a phosphate, phosphorothioate, or phosphorodithioate. Accordingly, it is to be understood that the phosphoesters and phosphodiesters described as having one or more of R^{E1}, R^{E2}, and R^{E3} as hydrogen encompass salts, in which the phosphate, phosphorothioate, or phosphorodithioate is present in a deprotonated, ionic form. In addition, the terms “free,” “naked,” and “unconjugated” referring to immunomodulating oligonucleotides, immunostimulating oligonucleotides, immunosuppressive oligonucleotides, and/or oligonucleotides (e.g., CpG oligonucleotides) may be used interchangeably herein.

The term “phosphotriester,” as used herein, refers to a phosphoester, in which all three valences are substituted with non-hydrogen substituents. The phosphotriester consists of phosphate, phosphorothioate, or phosphorodithioate; one or two bonds to nucleoside(s), or abasic spacer(s), and/or phosphoryl group(s); and one or two groups independently selected from the group consisting of a bioreversible group; a non-bioreversible group; an auxiliary moiety; a conjugating group; and a linker bonded to a targeting moiety and optionally to one or more (e.g., 1 to 6) auxiliary moieties. A terminal phosphotriester includes one bond to a group containing a nucleoside and two groups independently selected from the group consisting of a bioreversible group; a non-bioreversible group; an auxiliary moiety; a conjugating group; a phosphoryl group; and a linker bonded to a targeting moiety and optionally to one or more (e.g., 1 to 6) auxiliary moieties. In some embodiments, a terminal phosphotriester contains 1 or 0 linkers bonded to a targeting moiety and optionally to one or more (e.g., 1 to 6) auxiliary moieties. An internucleoside phosphotriester includes two bonds to nucleoside-containing groups. A phosphotriester may be a group of the following structure:

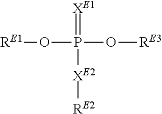

wherein:
each of X^{E1} and X^{E2} is independently O or S;
each or R^{E1} and R^{E3} is independently a bond to a nucleoside; a sugar analogue of an abasic spacer; a bioreversible group; a non-bioreversible group; an auxiliary moiety; a conjugating group; a linker bonded to a targeting moiety; a linker bonded to a targeting moiety and one or more (e.g., 1 to 6) auxiliary moieties; or the phosphorus atom in a group of formula —P(=XE1)(—X^{E2}-R^{E2}A)-O⁻,
   where R^{E2}A is hydrogen; a bioreversible group; a non-bioreversible group; an auxiliary moiety; a conjugating group; a linker bonded to a targeting moiety; or a linker bonded to a targeting moiety and one or more (e.g., 1 to 6) auxiliary moieties; and
R^{E2} is a bioreversible group; a non-bioreversible group; an auxiliary moiety; a conjugating group; a linker bonded to a targeting moiety; or a linker bonded to a targeting moiety and one or more (e.g., 1 to 6) auxiliary moieties;
provided that at least one of R^{E1} and R^{E3} is a bond to a group containing at least one nucleoside.
   If both R^{E1} and R^{E3} are bonds to groups containing at least one nucleoside, the phosphotriester is an internucleoside phosphotriester. If one and only one of R^{E1} and R^{E3} is a bond to a group containing a nucleoside, the phosphotriester is a terminal phosphotriester.

As used herein, the term “amino acid” refers to any amino acid (both standard and non-standard amino acids), including, but not limited to, α-amino acids, 3-amino acids, T-amino acids and 6-amino acids. Examples of suitable amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of suitable amino acids include, but are not limited to, omithine, hypusine, 2-aminoisobutyric acid, dehydroalanine, gamma-aminobutyric acid, citrulline, beta-alanine, alpha-ethyl-glycine, alpha-propyl-glycine and norleucine.

The terms “antibody,” “immunoglobulin,” and “Ig” are used interchangeably herein, and are used in the broadest sense and specifically cover, for example, individual monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments of antibodies. An antibody can be human, humanized, chimeric and/or affinity matured as well as an antibody from other species, for example, mouse and rabbit.

The term “antibody” is intended to include a polypeptide product of B cells within the immunoglobulin class of polypeptides that is able to bind to a specific antigen and is composed of two identical pairs of polypeptide chains, wherein each pair has one heavy chain (about 50-70 kDa) and one light chain (about 25 kDa) and each amino-terminal portion of each chain includes a variable region of about 100 to about 130 or more amino acids and each carboxyl-terminal portion of each chain includes a constant region. See Borrebaeck (ed.) (1995) Antibody Engineering, Second Ed., Oxford University Press.; Kuby (1997) Immunology, Third Ed., W. H. Freeman and Company, New York. Antibodies also include, but are not limited to, synthetic antibodies, monoclonal antibodies, recombinant antibodies, multispecific antibodies (including bispecific antibodies), human antibodies, humanized antibodies, camelized antibodies, chimeric antibodies, intrabodies, anti-idiotypic (anti-Id) antibodies, and functional fragments thereof, which refers a portion of an antibody heavy or light chain polypeptide that retains some or all of the binding activity of the antibody from which the fragment is derived. Non-limiting examples of functional fragments of an antibody include single-chain Fvs (scFv) (e.g., including monospecific or bispecific), Fab fragments, F(ab′) fragments, F(ab)₂ fragments, F(ab′)₂ fragments, disulfide-linked Fvs (sdFv), Fd fragments, Fv fragments, scRv-Fc, nanobody, diabody, triabody, tetrabody, and minibody. In some embodiments, the antibody comprises an Fc variant that has reduced or ablated effector function. In particular, antibodies provided herein include immunoglobulin molecules and immunologically active portions of immunoglobulin molecules, for example, antigen-binding domains or molecules that contain an antigen-binding site that binds to the antigen (e.g., one or more complementarity determining regions (CDRs) of an anti-CD56 antibody or an anti-SIRPα antibody). Such antibody fragments are described in, for example, Harlow and Lane, Antibodies: A Laboratory Manual, Cold Spring Harbor Laboratory, New York (1989); Myers (ed.), Molec. Biology and Biotechnology: A Comprehensive Desk Reference, New York: VCH Publisher, Inc.; Huston et al., Cell Biophysics 1993, 22, 189-224; Plickthun and Skerra, Meth. Enzymol. 1989, 178, 497-515; and Day, Advanced Immunochemistry, Second Ed., Wiley-Liss, Inc., New York, NY (1990). The antibodies provided herein can be of any type (e.g., IgG, IgE, IgM, IgD, IgA, and IgY), any class (e.g., IgG1, IgG2, IgG3, IgG4, IgA1, and IgA2), or any subclass (e.g., IgG2a and IgG2b) of an immunoglobulin molecule.

The term “antigen” refers to a predetermined target to which an antibody can selectively bind. A target antigen can be a polypeptide, carbohydrate, nucleic acid, lipid, hapten, or fragment thereof, or other naturally occurring or synthetic compound. In one embodiment, the target antigen is a polypeptide.

The terms “antigen-binding fragment,” “antigen-binding domain,” and “antigen-binding region” refer to a portion of an antibody that comprises the amino acid residues that interact with an antigen (e.g., a polypeptide, carbohydrate, nucleic acid, lipid, hapten, or fragment thereof, or other naturally occurring or synthetic compound) and confer on the binding agent its specificity and affinity for the antigen (e.g., complementarity determining regions (CDRs)).

The term “specific binding,” “specifically binds to,” or “specific for” a particular polypeptide or an epitope on a particular polypeptide target can be exhibited, for example, by a molecule (e.g., an antibody) having a dissociation constant (K_{d}) for the target of at least about 10⁻⁴ M, at least about 10⁻⁵ M, at least about 10⁻⁶ M, at least about 10⁻⁷ M, at least about 10⁻⁸ M, at least about 10⁻⁹ M, at least about 10⁻¹⁰ M, at least about 10⁻¹¹ M, or at least about 10⁻¹² M. In one embodiment, the term “specific binding” refers to binding where a molecule binds to a particular polypeptide or epitope on a particular polypeptide without substantially binding to any other polypeptide or polypeptide epitope.

A 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the a and 7 chains and four CH domains for and F isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, e.g., Basic and Clinical Immunology, 8th edition, Stites et al. (eds.), Appleton & Lange, Norwalk, CT, 1994, page 71 and Chapter 6.

The term “variable region” or “variable domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a R sheet configuration, connected by three hypervariable regions, which form loops connecting, and in some cases forming part of, the R sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, M D, 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. The variability in sequence is concentrated in the CDRs while the less variable portions in the variable region are referred to as framework regions (FR). The CDRs of the light and heavy chains are primarily responsible for the interaction of the antibody with antigen. In specific embodiments, the variable region is a human variable region.

The term “variable region residue numbering as in Kabat” or “amino acid position numbering as in Kabat”, and variations thereof, refers to the numbering system used for heavy chain variable regions or light chain variable regions of the compilation of antibodies in Kabat et al., Sequences of Proteins of Immunological Interest, 5th Ed. Public Health Service, National Institutes of Health, Bethesda, MD. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, including, for example, by AbM, Chothia, Contact, IMGT and AHon.

An “intact” antibody is one comprising an antigen-binding site as well as a CL and at least heavy chain constant regions, CH1, CH₂ and CH3. The constant regions may include human constant regions or amino acid sequence variants thereof. Preferably, an intact antibody has one or more effector functions.

The term “antibody fragment” refers to a portion of an intact antibody, preferably the antigen-binding or variable region of the intact antibody. Examples of antibody fragments include, without limitation, Fab, Fab′, F(ab′)₂, and Fv fragments; diabodies and di-diabodies (see, e.g., Holliger et al., Proc. Natl. Acad. Sci. U.S.A. 1993, 90, 6444-8; Lu et al., J. Biol. Chem. 2005, 280, 19665-72; Hudson et al., Nat. Med. 2003, 9, 129-134; WO 93/11161; and U.S. Pat. Nos. 5,837,242 and 6,492,123); single-chain antibody molecules (see, e.g., U.S. Pat. Nos. 4,946,778; 5,260,203; 5,482,858 and 5,476,786); dual variable domain antibodies (see, e.g., U.S. Pat. No. 7,612,181); single variable domain antibodies (SdAbs) (see, e.g., Woolven et al., Immunogenetics 1999, 50, 98-101 Streltsov et al., Proc. Natl. Acad. Sci. U.S.A. 2004, 101, 12444-12449); and multispecific antibodies formed from antibody fragments.

The term “functional fragment,” “binding fragment,” or “antigen-binding fragment” of an antibody refers to a molecule that exhibits at least one of the biological functions attributed to the intact antibody, the function comprising at least binding to the target antigen.

The term “heavy chain” when used in reference to an antibody refers to a polypeptide chain of about 50-70 kDa, wherein the amino-terminal portion includes a variable region of about 120 to 130 or more amino acids and a carboxyl-terminal portion that includes a constant region. The constant region can be one of five distinct types, (e.g., isotypes) referred to as alpha (a), delta (6), epsilon (s), gamma (7) and mu (p), based on the amino acid sequence of the heavy chain constant region. The distinct heavy chains differ in size: a, 6 and 7 contain approximately 450 amino acids, while p and F contain approximately 550 amino acids. When combined with a light chain, these distinct types of heavy chains give rise to five well known classes (e.g., isotypes) of antibodies, IgA, IgD, IgE, IgG and IgM, respectively, including four subclasses of IgG, namely IgG1, IgG2, IgG3, and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids and a carboxyl-terminal portion that includes a constant region. The approximate length of a light chain is 211 to 217 amino acids. There are two distinct types, referred to as kappa (x) of lambda (k) based on the amino acid sequence of the constant domains. Light chain amino acid sequences are well known in the art. A light chain can be a human light chain.

The term “monoclonal antibody” as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell, wherein the antibody binds to only a beta klotho epitope as determined, for example, by ELISA or other antigen-binding or competitive binding assay known in the art. The term “monoclonal” is not limited to any particular method for making the antibody. For example, the monoclonal antibodies useful in the present disclosure may be prepared by the hybridoma methodology first described by Kohler et al., Nature 1975, 256, 495; or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature 1991, 352, 624-628 and Marks et al., J. Mol. Biol. 1991, 222, 581-597, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel et al., eds., John Wiley and Sons, New York). Exemplary methods of producing monoclonal antibodies are provided in the Examples herein.

“Humanized” forms of nonhuman (e.g., murine) antibodies are chimeric antibodies that include human immunoglobulins (e.g., recipient antibody) in which the native CDR residues are replaced by residues from the corresponding CDR of a nonhuman species (e.g., donor antibody) such as mouse, rat, rabbit or nonhuman primate having the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 1986, 321, 522-525; Riechmann et al., Nature 1988, 332, 323-329; Presta, Curr. Opin. Biotechnol. 1992, 3, 394-398; Carter et al., Proc. Natl. Acad. Sci. U.S.A. 1992, 89, 4285-4289; and U.S. Pat. Nos. 6,800,738, 6,719,971, 6,639,055, 6,407,213, and 6,054,297.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 1991, 227, 381; Marks et al., J. Mol. Biol. 1991, 222, 581) and yeast display libraries (Chao et al., Nature Protocols 2006, 1, 755-768). Also available for the preparation of human monoclonal antibodies are methods described in Cole et al., Monoclonal Antibodies and Cancer Therapy, Alan R. Liss, p. 77 (1985); Boerner et al., J. Immunol. 1991, 147, 86-95. See also van Dijk and van de Winkel, Curr. Opin. Pharmacol. 2001, 5, 368-374. Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 1995, 6, 561-566; Bruggemann and Taussing, Curr. Opin. Biotechnol. 1997, 8, 455-458; and U.S. Pat. Nos. 6,075,181 and 6,150,584 regarding XENOMOUSE™ technology). See also, for example, Li et al., Proc. Natl. Acad. Sci. U.S.A. 2006, 103, 3557-3562 regarding human antibodies generated via a human B-cell hybridoma technology.

A “CDR” refers to one of three hypervariable regions (H1, H2, or H3) within the non-framework region of the immunoglobulin (Ig or antibody) VH j-sheet framework, or one of three hypervariable regions (L1, L2, or L3) within the non-framework region of the antibody VL j-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains. Kabat et al., J. Biol. Chem. 1977, 252, 6609-6616; Kabat, Adv. Protein Chem. 1978, 32, 1-75. CDR region sequences also have been defined structurally by Chothia as those residues that are not part of the conserved β-sheet framework, and thus are able to adapt different conformations. Chothia and Lesk, J. Mol. Biol. 1987, 196, 901-917. Both terminologies are well recognized in the art. CDR region sequences have also been defined by AbM, Contact and IMGT. The positions of CDRs within a canonical antibody variable region have been determined by comparison of numerous structures. Al-Lazikani et al., J. Mol. Biol. 1997, 273, 927-948; Morea et al., Methods. 2000, 20, 267-279. Because the number of residues within a hypervariable region varies in different antibodies, additional residues relative to the canonical positions are conventionally numbered with a, b, c and so forth next to the residue number in the canonical variable region numbering scheme. Al-Lazikani et al., supra (1997). Such nomenclature is similarly well known to those skilled in the art.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

“Cycloalkyl” indicates a non-aromatic, fully saturated carbocyclic ring having the indicated number of carbon atoms, for example, 3 to 10, or 3 to 8, or 3 to 6 ring carbon atoms. Cycloalkyl groups may be monocyclic or polycyclic (e.g., bicyclic, tricyclic). Examples of cycloalkyl groups include cyclopropyl, cyclobutyl, cyclopentyl, and cyclohexyl, as well as bridged and caged ring groups (e.g., norbornane, bicyclo[2.2.2]octane). In addition, one ring of a polycyclic cycloalkyl group may be aromatic, provided the polycyclic cycloalkyl group is bound to the parent structure via a non-aromatic carbon. For example, a 1,2,3,4-tetrahydronaphthalen-1-yl group (wherein the moiety is bound to the parent structure via a non-aromatic carbon atom) is a cycloalkyl group, while 1,2,3,4-tetrahydronaphthalen-5-yl (wherein the moiety is bound to the parent structure via an aromatic carbon atom) is not considered a cycloalkyl group. Examples of polycyclic cycloalkyl groups consisting of a cycloalkyl group fused to an aromatic ring are described below.

“Cycloalkenyl” indicates a non-aromatic carbocyclic ring, containing the indicated number of carbon atoms (e.g., 3 to 10, or 3 to 8, or 3 to 6 ring carbon atoms) and at least one carbon-carbon double bond. Cycloalkenyl groups may be monocyclic or polycyclic (e.g., bicyclic, tricyclic). Examples of cycloalkenyl groups include cyclopropenyl, cyclobutenyl, cyclopentenyl, cyclopentadienyl, and cyclohexenyl, as well as bridged and caged ring groups (e.g., bicyclo[2.2.2]octene). In addition, one ring of a polycyclic cycloalkenyl group may be aromatic, provided the polycyclic alkenyl group is bound to the parent structure via a non-aromatic carbon atom. For example, inden-1-yl (wherein the moiety is bound to the parent structure via a non-aromatic carbon atom) is considered a cycloalkenyl group, while inden-4-yl (wherein the moiety is bound to the parent structure via an aromatic carbon atom) is not considered a cycloalkenyl group. Examples of polycyclic cycloalkenyl groups consisting of a cycloalkenyl group fused to an aromatic ring are described below.

“Cycloalkynyl” refers to an unsaturated hydrocarbon group within a cycloalkyl having at least one site of acetylenic unsaturation (i.e., having at least one moiety of the formula C≡C). Cycloalkynyl can consist of one ring, such as cyclooctyne, or multiple rings. One cycloalkynyl moiety is an unsaturated cyclic hydrocarbon having from 5 to 10 annular carbon atoms (a “C₅-C₁₀ cycloalkynyl”). Examples include cyclopentyne, cyclohexyne, cycloheptyne, cyclooctyne, cyclononyne, and the like.

“Aryl” indicates an aromatic carbocyclic ring having the indicated number of carbon atoms, for example, 6 to 12 or 6 to 10 carbon atoms. Aryl groups may be monocyclic or polycyclic (e.g., bicyclic, tricyclic). In some instances, both rings of a polycyclic aryl group are aromatic (e.g., naphthyl). In other instances, polycyclic aryl groups may include a non-aromatic ring fused to an aromatic ring, provided the polycyclic aryl group is bound to the parent structure via an atom in the aromatic ring. Thus, a 1,2,3,4-tetrahydronaphthalen-5-yl group (wherein the moiety is bound to the parent structure via an aromatic carbon atom) is considered an aryl group, while 1,2,3,4-tetrahydronaphthalen-1-yl (wherein the moiety is bound to the parent structure via a non-aromatic carbon atom) is not considered an aryl group. Similarly, a 1,2,3,4-tetrahydroquinolin-8-yl group (wherein the moiety is bound to the parent structure via an aromatic carbon atom) is considered an aryl group, while 1,2,3,4-tetrahydroquinolin-1-yl group (wherein the moiety is bound to the parent structure via a non-aromatic nitrogen atom) is not considered an aryl group. However, the term “aryl” does not encompass or overlap with “heteroaryl”, as defined herein, regardless of the point of attachment (e.g., both quinolin-5-yl and quinolin-2-yl are heteroaryl groups). In some instances, aryl is phenyl or naphthyl. In certain instances, aryl is phenyl. Additional examples of aryl groups comprising an aromatic carbon ring fused to a non-aromatic ring are described below.

The term “DAR” refers to a drug-antibody ratio of an oligonucleotide-antibody conjugate, more specifically an immunomodulating oligonucleotide-antibody ratio. In some instances, for example, an oligonucleotide-antibody conjugate may be described herein as having a DAR of 1 or as a DAR1 conjugate, wherein the oligonucleotide-antibody ratio is 1-to-1. In other instances, an an oligonucleotide-antibody conjugate may be described herein as having a DAR of 2 or as a DAR2 conjugate, wherein the oligonucleotide-antibody ratio is 2-to-1.

“Heteroaryl” indicates an aromatic ring containing the indicated number of atoms (e.g., 5 to 12, or 5 to 10 membered heteroaryl) made up of one or more heteroatoms (e.g., 1, 2, 3 or 4 heteroatoms) selected from N, O and S and with the remaining ring atoms being carbon. Heteroaryl groups do not contain adjacent S and O atoms. In some embodiments, the total number of S and O atoms in the heteroaryl group is not more than 2. In some embodiments, the total number of S and O atoms in the heteroaryl group is not more than 1. Unless otherwise indicated, heteroaryl groups may be bound to the parent structure by a carbon or nitrogen atom, as valency permits. For example, “pyridyl” includes 2-pyridyl, 3-pyridyl and 4-pyridyl groups, and “pyrrolyl” includes 1-pyrrolyl, 2-pyrrolyl and 3-pyrrolyl groups.

In some instances, a heteroaryl group is monocyclic. Examples include pyrrole, pyrazole, imidazole, triazole (e.g., 1,2,3-triazole, 1,2,4-triazole, 1,2,4-triazole), tetrazole, furan, isoxazole, oxazole, oxadiazole (e.g., 1,2,3-oxadiazole, 1,2,4-oxadiazole, 1,3,4-oxadiazole), thiophene, isothiazole, thiazole, thiadiazole (e.g., 1,2,3-thiadiazole, 1,2,4-thiadiazole, 1,3,4-thiadiazole), pyridine, pyridazine, pyrimidine, pyrazine, triazine (e.g., 1,2,4-triazine, 1,3,5-triazine) and tetrazine.

In some instances, both rings of a polycyclic heteroaryl group are aromatic. Examples include indole, isoindole, indazole, benzoimidazole, benzotriazole, benzofuran, benzoxazole, benzoisoxazole, benzoxadiazole, benzothiophene, benzothiazole, benzoisothiazole, benzothiadiazole, 1H-pyrrolo[2,3-b]pyridine, 1H-pyrazolo[3,4-b]pyridine, 3H-imidazo[4,5-b]pyridine, 3H-[1,2,3]triazolo[4,5-b]pyridine, 1H-pyrrolo[3,2-b]pyridine, 1H-pyrazolo[4,3-b]pyridine, 1H-imidazo[4,5-b]pyridine, 1H-[1,2,3]triazolo[4,5-b]pyridine, 1H-pyrrolo[2,3-c]pyridine, 1H-pyrazolo[3,4-c]pyridine, 3H-imidazo[4,5-c]pyridine, 3H-[1,2,3]triazolo[4,5-c]pyridine, 1H-pyrrolo[3,2-c]pyridine, 1H-pyrazolo[4,3-c]pyridine, 1H-imidazo[4,5-c]pyridine, 1H-[1,2,3]triazolo[4,5-c]pyridine, furo[2,3-b]pyridine, oxazolo[5,4-b]pyridine, isoxazolo[5,4-b]pyridine, [1,2,3]oxadiazolo[5,4-b]pyridine, furo[3,2-b]pyridine, oxazolo[4,5-b]pyridine, isoxazolo[4,5-b]pyridine, [1,2,3]oxadiazolo[4,5-b]pyridine, furo[2,3-c]pyridine, oxazolo[5,4-c]pyridine, isoxazolo[5,4-c]pyridine, [1,2,3]oxadiazolo[5,4-c]pyridine, furo[3,2-c]pyridine, oxazolo[4,5-c]pyridine, isoxazolo[4,5-c]pyridine, [1,2,3]oxadiazolo[4,5-c]pyridine, thieno[2,3-b]pyridine, thiazolo[5,4-b]pyridine, isothiazolo[5,4-b]pyridine, [1,2,3]thiadiazolo[5,4-b]pyridine, thieno[3,2-b]pyridine, thiazolo[4,5-b]pyridine, isothiazolo[4,5-b]pyridine, [1,2,3]thiadiazolo[4,5-b]pyridine, thieno[2,3-c]pyridine, thiazolo[5,4-c]pyridine, isothiazolo[5,4-c]pyridine, [1,2,3]thiadiazolo[5,4-c]pyridine, thieno[3,2-c]pyridine, thiazolo[4,5-c]pyridine, isothiazolo[4,5-c]pyridine, [1,2,3]thiadiazolo[4,5-c]pyridine, quinoline, isoquinoline, cinnoline, quinazoline, quinoxaline, phthalazine, naphthyridine (e.g., 1,8-naphthyridine, 1,7-naphthyridine, 1,6-naphthyridine, 1,5-naphthyridine, 2,7-naphthyridine, 2,6-naphthyridine), imidazo[1,2-a]pyridine, 1H-pyrazolo[3,4-d]thiazole, 1H-pyrazolo[4,3-d]thiazole and imidazo[2,1-b]thiazole.

In other instances, polycyclic heteroaryl groups may include a non-aromatic ring (e.g., cycloalkyl, cycloalkenyl, heterocycloalkyl, heterocycloalkenyl) fused to a heteroaryl ring, provided the polycyclic heteroaryl group is bound to the parent structure via an atom in the aromatic ring. For example, a 4,5,6,7-tetrahydrobenzo[d]thiazol-2-yl group (wherein the moiety is bound to the parent structure via an aromatic carbon atom) is considered a heteroaryl group, while 4,5,6,7-tetrahydrobenzo[d]thiazol-5-yl (wherein the moiety is bound to the parent structure via a non-aromatic carbon atom) is not considered a heteroaryl group. Examples of polycyclic heteroaryl groups consisting of a heteroaryl ring fused to a non-aromatic ring are described below.

As used herein, the terms “including,” “containing,” and “comprising” are used in their open, non-limiting sense. It is also understood that aspects and embodiments of the invention described herein may include “consisting” and/or “consisting essentially of” aspects and embodiments.

It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

As used herein, a “carrier” includes pharmaceutically acceptable carriers, excipients, or stabilizers that are nontoxic to the cell or mammal being exposed thereto at the dosages and concentrations employed. Often the physiologically acceptable carrier is an aqueous pH buffered solution. Non-limiting examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

As used herein, the term “effective amount” or “therapeutically effective amount” of a substance is at least the minimum concentration required to bring about a measurable improvement or prevention of a particular disorder. An effective amount herein may vary according to factors such as the disease state, age, sex, and weight of the patient, and the ability of the substance to elicit a desired response in the individual. An effective amount is also one in which any toxic or detrimental effects of the treatment are outweighed by the therapeutically beneficial effects. In reference to cancer, an effective amount comprises an amount sufficient to cause a tumor to shrink and/or to decrease the growth rate of the tumor (such as to suppress tumor growth) or to prevent or delay other unwanted cell proliferation in cancer. In some embodiments, an effective amount is an amount sufficient to delay development of cancer. In some embodiments, an effective amount is an amount sufficient to prevent or delay recurrence. In some embodiments, an effective amount is an amount sufficient to reduce recurrence rate in the individual. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) reduce recurrence rate of tumor, and/or (viii) relieve to some extent one or more of the symptoms associated with the cancer. An effective amount can be administered in one or more administrations. For purposes of this disclosure, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments, etc.

The terms “protein,” “polypeptide” and “peptide” are used herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Typically, a protein for use herein will have a molecular weight of at least about 5-20 kDa, alternatively at least about 20-100 kDa, or at least about 100 kDa. Also included within the definition are, for example, proteins containing one or more analogs of an amino acid (including, for example, unnatural amino acids, etc.), as well as other modifications known in the art.

A “pharmaceutically acceptable salt” is a salt form that is non-toxic, biologically tolerable, or otherwise biologically suitable for administration to the subject. See generally Berge et al. (1977) J. Pharm. Sci. 66, 1. Particular pharmaceutically acceptable salts are those that are pharmacologically effective and suitable for contact with the tissues of subjects without undue toxicity, irritation, or allergic response. Pharmaceutically acceptable salts include, without limitation, acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like. These salts may be derived from inorganic or organic acids. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, 7-hydroxybutyrates, glycolates, tartrates, and mandelates. In some embodiments, pharmaceutically acceptable salts are formed when an acidic proton present in the parent compound either is replaced by a metal ion, e.g., an alkali metal ion, an alkaline earth ion, or an aluminum ion; or coordinates with an organic base. Salts derived from pharmaceutically acceptable organic non-toxic bases include, without limitation, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, tromethamine, trimetharnine, dicyclohexylamine, caffeine, procaine, hydrabamine, choline, betaine, ethylenediamine, glucosamine, N-ethylglucamine, N-methylglucamine, theobromine, purines, piperazine, piperidine, N-ethylpiperidine, polyamine resins, amino acids such as lysine, arginine, histidine, and the like. Examples of pharmaceutically acceptable base addition salts include those derived from inorganic bases such as sodium, potassium, lithium, ammonium, calcium, magnesium, iron, zinc, copper, manganese, aluminum salts and the like. In some embodiments, the organic non-toxic bases are L-amino acids, such as L-lysine and L-arginine, tromethamine, N-ethylglucamine and N-methylglucamine. Acceptable inorganic bases include, without limitation, aluminum hydroxide, calcium hydroxide, potassium hydroxide, sodium carbonate, sodium hydroxide, and the like. Lists of other suitable pharmaceutically acceptable salts are found in Remington's Pharmaceutical Sciences, 17th Edition, Mack Publishing Company, Easton, Pa., 1985.

A “solvate” is formed by the interaction of a solvent and a compound. Suitable solvents include, for example, water and alcohols (e.g., ethanol). Solvates include hydrates having any ratio of compound to water, such as monohydrates, dihydrates and hemi-hydrates.

A “subject,” “patient” or “individual” includes a mammal, such as a human or other animal, and typically is human. In some embodiments, the subject, e.g., patient, to whom the therapeutic agents and compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent, a dog, a cat, a farm animal, such as a cow or a horse, etc.

The term “cancer” or “tumor” refers to the presence of cells possessing characteristics typical of cancer-causing cells, such as uncontrolled proliferation, immortality, metastatic potential, rapid growth and proliferation rate, and certain characteristic morphological features. Cancer cells are often in the form of a solid tumor, which is detectable on the basis of tumor mass, e.g., by procedures such as CAT scan, MR imaging, X-ray, ultrasound or palpation, and/or which is detectable because of the expression of one or more cancer-specific antigens in a sample obtainable from a patient. In some embodiments, a solid tumor does not need to have measurable dimensions. Cancer cells may also in the form of a liquid tumor, which cancer cells may exist alone or disseminated within an animal. As used herein, the terms “disseminated tumor” and “liquid tumor” are used interchangeably, and include, without limitation, leukemia and lymphoma and other blood cell cancers.

The term “leukemia” refers to a type of cancer of the blood or bone marrow characterized by an abnormal increase of immature white blood cells called “blasts.” Leukemia is a broad term covering a spectrum of diseases. In turn, it is part of the even broader group of diseases affecting the blood, bone marrow, and lymphoid system, which are all known as hematological neoplasms. Leukemias can be divided into four major classifications, acute lymphocytic (or lymphoblastic) leukemia (ALL), acute myelogenous (or myeloid or non-lymphatic) leukemia (AML), chronic lymphocytic leukemia (CLL), and chronic myelogenous leukemia (CML). Further types of leukemia include Hairy cell leukemia (HCL), T-cell prolymphocytic leukemia (T-PLL), large granular lymphocytic leukemia, and adult T-cell leukemia.

The term “lymphoma” refers to a group of blood cell tumors that develop from lymphatic cells. The two main categories of lymphomas are Hodgkin lymphomas (HL) and non-Hodgkin lymphomas (NHL) Lymphomas include any neoplasms of the lymphatic tissues. The main classes are cancers of the lymphocytes, a type of white blood cell that belongs to both the lymph and the blood and pervades both.

As used herein, the term “cancer” includes premalignant as well as malignant cancers, and also includes primary tumors (e.g., those whose cells have not migrated to sites in the subject's body other than the site of the original tumor) and secondary tumors (e.g., those arising from metastasis, the migration of tumor cells to secondary sites that are different from the site of the original tumor), recurrent cancer and refractory cancer.

The terms “cancer recurrence” and “cancer relapse” are used interchangeably and refer to the return of a sign, symptom or disease after a remission. The recurrent cancer cells may re-appear in the same site of the primary tumor or in another location, such as in secondary cancer. The cancer cells may re-appear in the same diseased form as the primary cancer or a different diseased form. For example, in some embodiments, a primary cancer is a solid tumor, and the recurrent cancer is a liquid tumor. In other embodiments, a primary cancer is a liquid tumor, and the recurrent cancer is a solid tumor. In yet other embodiments, the primary cancer and the recurrent cancer are both solid tumors, or both liquid tumors. In some embodiments, the recurrent tumor expresses at least one tumor-associated antigen that is also expressed by the primary tumor.

The term “refractory cancer” as used herein refers to a cancer that does not respond to a treatment, for example, a cancer that is resistant at the beginning of treatment (e.g., treatment with an immunotherapy) or a cancer that may become resistant during treatment. The terms “respond,” “response” or “responsiveness” refer to an anti-cancer response, e.g. in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

As used herein, cancers include, but are not limited to, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, B cell cancer, e.g., multiple myeloma, Waldenstram's macroglobulinemia, the heavy chain diseases, such as, for example, alpha chain disease, gamma chain disease, and mu chain disease, benign monoclonal qammopathy, and immunocytic amyloidosis, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, sominoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

The term “cancer therapy” or “cancer therapeutic agent” as used herein, refers to those therapies or agents that can exert anti-tumor effect or have an anti-tumor activity. Such anti-tumor effect or anti-tumor activity can be exhibited as a reduction in the rate of tumor cell proliferation, viability, or metastatic activity. A possible way of showing anti-tumor activity is to show a decline in growth rate of abnormal cells that arises during therapy or tumor size stability or reduction. Such activity can be assessed using accepted in vitro or in vivo tumor models, including but not limited to xenograft models, allograft models, MMTV models, and other known models known in the art to investigate anti-tumor activity.

The terms “treat,” “treating,” and “treatment” are meant to include alleviating or abrogating a condition, disorder, or disease, or one or more of the symptoms associated with the condition, disorder, or disease; or alleviating or eradicating the cause(s) of the condition, disorder, or disease itself.

The terms “prevent,” “preventing,” and “prevention” are meant to include a method of delaying and/or precluding the onset of a condition, disorder, or disease, and/or its attendant symptoms; barring a subject from acquiring a condition, disorder, or disease; or reducing a subject's risk of acquiring a condition, disorder, or disease.

The term “substituted” means that the specified group or moiety bears one or more substituents including, but not limited to, substituents such as alkoxy, acyl, acyloxy, alkoxycarbonyl, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, alkyl, alkenyl, alkynyl, heterocyclyl, aralkyl, aminosulfonyl, sulfonylamino, sulfonyl, oxo, and the like. The term “unsubstituted” means that the specified group bears no substituents. Where the term “substituted” is used to describe a structural system, the substitution is meant to occur at any valency-allowed position on the system. When a group or moiety bears more than one substituent, it is understood that the substituents may be the same or different from one another. In some embodiments, a substituted group or moiety bears from one to five substituents. In some embodiments, a substituted group or moiety bears one substituent. In some embodiments, a substituted group or moiety bears two substituents. In some embodiments, a substituted group or moiety bears three substituents. In some embodiments, a substituted group or moiety bears four substituents. In some embodiments, a substituted group or moiety bears five substituents.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible, and/or inherently unstable. It will also be understood that where a group or moiety is optionally substituted, the disclosure includes both embodiments in which the group or moiety is substituted and embodiments in which the group or moiety is unsubstituted.

The term “Q-tag,” as used herein, refers to a portion of a polypeptide containing glutamine residue that, upon transglutaminase-mediated reaction with a compound containing —NH₂ amine, provides a conjugate containing the portion of polypeptide, in which the glutamine residue includes a side chain modified to include the amide bonded to the compound. Q-tags are known in the art. Non-limiting examples of Q-tags are LLQGG (SEQ ID NO:172), GGGLLQGG (SEQ ID NO:173), RPQGF (SEQ ID NO:47), RPQGFPP (SEQ ID NO:48), RPQGFGPP (SEQ ID NO:49), and Q-tags disclosed in Table 16 of this disclosure. In some embodiments, the Q tag is attached to the C terminal of the heavy chain of the antibody. In some embodiments, the Q tag is attached to the light chain of the antibody. In some embodiments, the Q tag is naturally occurring. For example, mutation of N297 to N297A exposes Q295 of the antibody, where the conjugation could occur (numbering according to EU index, e.g., as listed in Edelman, G. M. et al., Proc. Natl. Acad. USA, 63, 78-85 (1969) and Kabat, E. A. et al., Sequences of proteins of immunological interest. 5th Edition—US Department of Health and Human Services, NIH publication n° 91-3242, pp 662,680,689 (1991)). In some embodiments, the Q tag is within the Fc domain of the antibody.

Throughout this application, antibodies and conjugates are designated in the format “TNT-y” wherein “y” is a number (e.g., TNT-201) or a combination of a number and letters (e.g., “TNT-347xx”). The hyphen is provided simply for style and readability, and the hyphenated and unhyphenated designations are identical (e.g., “TNT-201” is the same construct as “TNT201”). Each antibody or conjugate may be defined by a variety of constituent sequences (e.g., TNT-201 includes at least sequences for CDR-L1, CDR-L2, CDR-L3, CDR-H1, CDR-H2, CDR-H3, VL, VH, heavy chain, and light chain).

### II. Conjugates

To improve targeting specificity and in vivo distribution, immunomodulating oligonucleotides (e.g., CpG ODNs) can be conjugated to a nectin-4 antibody. Any nectin-4 antibody (i.e., any antibody which specifically binds to nectin-4) may be conjugated to the immunomodulating oligonucleotides described herein. In particular, nectin-4 antibodies described in the section titled “Nectin-4 antibodies” of this disclosure may be used. Any immunomodulating oligonucleotide described herein may be used, including those described in the section titled “Immunomodulating oligonucleotides” of this disclosure. The nectin-4 antibodies may be conjugated to immunomodulating oligonucleotides or other agents (as otherwise described herein) using methods described herein or by other means known in the art, including those described in US 2018/0312536 A1, the contents of which are hereby incorporated by reference for all purposes. In some embodiments, the immunomodulating oligonucleotide of the conjugate comprises the structure of any one of formulas (C), (C′), (C″), (D), (D′), or (D″) as described throughout this disclosure. In some embodiments, the CpG sequence in the immunostimulating oligonucleotide is unmethylated.

Provided herein are nectin-4 antibody conjugates (i.e., also referred to herein as nectin-4 antibodies conjugated to CpG ODNs; nectin-4 antibody-conjugates, anti-nectin-4 antibody-conjugates, or just “conjugates”) wherein the CpG oligonucleotide and nectin-4 antibody are attached together via a linking moiety. In some embodiments, one nectin-4 antibody can be conjugated to one or more oligonucleotides. In some embodiments, the oligonucleotide-antibody conjugate is a conjugate comprising a nectin-4 antibody or antigen-binding fragment thereof and one or more immunomodulating oligonucleotides (P), wherein the antibody or antigen-binding fragment is linked to one or more Q-tag peptides (Q) comprising at least one glutamine residue, wherein each immunomodulating oligonucleotide is linked to a Q-tag peptide via an amide bond with the glutamine residue of the Q-tag peptide and a linker (L) as shown in Formula (A):

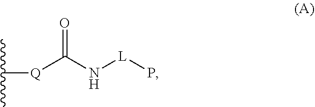

or a stereoisomer, a mixture of two or more diastereomers, a tautomer, or a mixture of two or more tautomers thereof; or a pharmaceutically acceptable salt, solvate, or hydrate thereof;
wherein:
[symbol] indicates the point of attachment of each Q to the antibody or antigen-binding fragment thereof (Ab);
each Q is independently a Q-tag peptide sequence comprising at least one glutamine residue;
each L is independently a bond or a linker moiety connected to Q via an amide bond with the glutamine residue; and
each P is independently an immunomodulating oligonucleotide.

In some embodiments, the conjugate is a conjugate comprising a nectin-4 antibody or antigen-binding fragment thereof and one or more immunomodulating oligonucleotides (P), wherein the nectin-4 antibody or antigen-binding fragment is linked to one or more Q-tag peptides (Q) that comprise the amino acid sequence RPQGF (SEQ ID NO:47), wherein each immunomodulating oligonucleotide is linked to a Q-tag peptide via an amide bond with the glutamine residue of the Q-tag peptide and a linker (L) as shown in Formula (A),

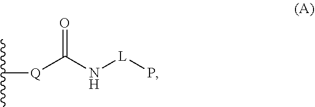

or a stereoisomer, a mixture of two or more diastereomers, a tautomer, or a mixture of two or more tautomers thereof; or a pharmaceutically acceptable salt, solvate, or hydrate thereof;
wherein:
[symbol] indicates the point of attachment of each Q to the antibody or antigen-binding fragment thereof (Ab);
each Q independently comprises a Q-tag peptide comprising a peptide sequence RPQGF (SEQ ID NO:47);
each L is independently a bond or a linker moiety connected to Q via an amide bond with the glutamine residue; and
each P is independently an immunomodulating oligonucleotide.

In some embodiments, the conjugate is a conjugate comprising a nectin-4 antibody or antigen-binding fragment thereof and one or more immunomodulating oligonucleotides (P), wherein the nectin-4 antibody or antigen-binding fragment is linked to one or more Q-tag peptides (Q) that comprise the amino acid sequence RPQGFGPP (SEQ ID NO:49), wherein each immunomodulating oligonucleotide is linked to a Q-tag peptide via an amide bond with the glutamine residue of the Q-tag peptide and a linker (L) as shown in Formula (A),

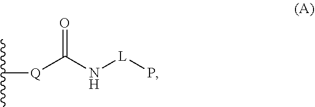

or a stereoisomer, a mixture of two or more diastereomers, a tautomer, or a mixture of two or more tautomers thereof; or a pharmaceutically acceptable salt, solvate, or hydrate thereof;
wherein:
[symbol] indicates the point of attachment of each Q to the antibody or antigen-binding fragment thereof (Ab);
each Q independently comprises a Q-tag peptide comprising a peptide sequence RPQGFGPP (SEQ ID NO:49);
each L is independently a bond or a linker moiety connected to Q via an amide bond with the glutamine residue; and
each P is independently an immunomodulating oligonucleotide.

In other embodiments, the conjugate is a conjugate comprising a nectin-4 antibody or antigen-binding fragment thereof and one or more immunomodulating oligonucleotides (P), wherein the antibody or antigen-binding fragment is linked to one or more Q-tag peptides (Q) comprising at least one glutamine residue, wherein each immunomodulating oligonucleotide is linked to a Q-tag peptide via an amide bond with the glutamine residue of the Q-tag peptide and a linker (L) as shown in formula (A),

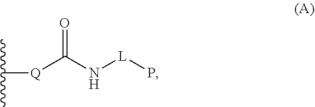

or a stereoisomer, a mixture of two or more diastereomers, a tautomer, or a mixture of two or more tautomers thereof; or a pharmaceutically acceptable salt, solvate, or hydrate thereof;
wherein:
[symbol] indicates the point of attachment of each Q to the antibody or antigen-binding fragment thereof (Ab);
each Q is independently a Q-tag peptide comprising at least one glutamine residue;
each L is independently a bond or a linker moiety connected to Q via an amide bond with the glutamine residue; and
each P is independently an immunomodulating oligonucleotide selected from the group consisting of the oligonucleotides of Table 4.

In one embodiment, the oligonucleotide-nectin-4 antibody conjugate has a DAR ranging from about 1 to about 20, from about 1 to about 10, from about 1 to about 8, from about 1 to about 4, or from about 1 to about 2. In another embodiment, the oligonucleotide-nectin-4 antibody conjugate has a DAR of about 1, about 2, about 3, about 4, about 5, about 6, about 7, or about 8.

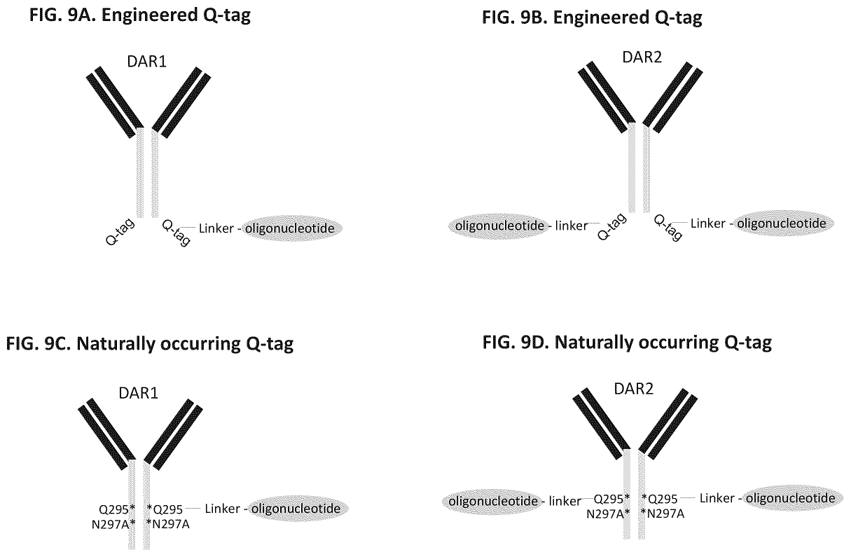

In some embodiments, the conjugate comprises one or more, two or more, three or more, four or more, five or more, six or more, seven or more, eight or more, nine or more, ten or more, or twenty or more Q-tag peptides. In some embodiments, the conjugate comprises one, two, three, four, five, six, seven, eight, nine, ten, or twenty Q-tag peptides. In some embodiments, the conjugate has 2 Q-tag peptides. In some embodiments, the conjugate comprises one or more, two or more, three or more, four or more, five or more, six or more, seven or more, eight or more, nine or more, ten or more, or twenty or more immunomodulating oligonucleotides. In some embodiments, the conjugate comprises one, two, three, four, five, six, seven, eight, nine, ten, or twenty immunomodulating oligonucleotides. In some embodiments, the conjugate has one immunomodulating oligonucleotide. An exemplary conjugate is shown in FIGS. 9A-9D which may comprise any nectin-4 antibody described herein (including any VH/VL described herein and/or any Fc region described herein), any linker described herein, any oligonucleotide described herein, and/or any Q-tag described herein.

In some embodiments, the conjugates as provided herein may also be prepared via conjugation of a nectin-4 antibody(ies) and one or more agents (such as one or more immunomodulating oligonucleotides) via a surface and/or exposed amino acid residues, such as lysines and/or cysteines, on the surface of the antibody and/or under suitable chemical conditions known in the art. In some embodiments, the agent(s) (such as one or more immunomodulating oligonucleotides) and/or the nectin-4 antibody(ies) is modified with a functional group capable of binding to a functional group present on the agent (such as the immunomodulating oligonucleotide) or antibody, or an amino acid residue exposed or present on the surface of the nectin-4 antibody. For example, in some embodiments, the agent(s) (such as one or more immunomodulating oligonucleotides) may be modified with a functional group capable of reacting with a lysine residue, such as via an acylation mechanism, including but not limited to N-hydroxysucinnamide-esters, iso(thio)cyanates, and benzoyl halides. In other embodiments, the agents(s) (such as one or more immunomodulating oligonucleotides) may be modified with a functional group capable of reacting with a cysteine residue, such as via formation of a disulfide bond, including but not limited to maleimides, haloacetamides, 2-thiopyridines, and 3-arylpropiolonitriles.

### II.A. IMMUNOMODULATING OLIGONUCLEOTIDES

In one aspect, the oligonucleotide in the nectin-4 antibody oligonucleotide-conjugate is an immunomodulating (e.g., immunostimulating) oligonucleotide. In certain embodiments, the immunomodulating oligonucleotide comprises a 5-modified uridine or 5-modified cytidine. In certain embodiments, the inclusion of 5-modified uridine (e.g., 5-ethynyl-uridine) at the 5′-terminus of the immunomodulating oligonucleotide (e.g., among the two 5′-terminal nucleosides) enhances the immunomodulating properties of the oligonucleotide. In certain embodiments, the immunomodulating oligonucleotide is shorter (e.g., comprising a total of from about 6 to about 16 nucleotides or from about 12 to about 14 nucleotides) than a typical CpG ODN, which is from 18 to 28 nucleotides in length. In certain embodiments, the shorter immunomodulating oligonucleotide (e.g., those comprising a total of from about 6 to about 16 nucleotides or from about 12 to about 14 nucleotides) retains the immunomodulating activity of a longer, typical CpG ODN; or exhibits higher immunomodulating activity (e.g., as measured by NFκB activation or by the changes in the expression levels of cell surface markers of activation or function such as CD40, HLADR, CD69 or CD80 or by the changes in the levels of at least one cytokine (e.g., IL-6 or IL-10), as compared to the longer CpG ODN. In certain embodiments, the immunomodulating oligonucleotide comprises an abasic spacer. In certain embodiments, the immunomodulating oligonucleotide comprises an internucleoside phosphotriester.

In certain embodiments, the immunomodulating oligonucleotide provided herein exhibits stability (e.g., stability against nucleases) that is superior to that of a CpG ODN containing mostly internucleoside phosphate (e.g., more than 50% of internucleoside phosphates) without substantially sacrificing its immunostimulating activity. This effect can be achieved, e.g., by incorporating at least 50% (e.g., at least 70%) internucleoside phosphorothioates or phosphorodithioates or through the inclusion of internucleoside phosphotriesters and/or internucleoside abasic spacers. Phosphotriesters and abasic spacers are also convenient for conjugation to a targeting moiety. Phosphate-based phosphotriesters and abasic spacers can also be used for reduction of off-target activity, relative to oligonucleotides with fully phosphorothioate backbones. Without wishing to be bound by theory, this effect may be achieved by reducing self-delivery without disrupting targeting moiety-mediated delivery to target cells. Accordingly, an oligonucleotide provided herein can include about 15 or fewer, about 14 or fewer, about 13 or fewer, about 12 or fewer, about 11 or fewer, or about 10 or fewer contiguous internucleoside phosphorothioates. For example, an immunostimulating oligonucleotide comprising a total of from about 12 to about 16 nucleosides can contain about 10 or fewer contiguous internucleoside phosphorothioates.

The immunostimulating oligonucleotide provided herein can contain a total of about 50 or fewer, about 30 or fewer, about 28 or fewer, or about 16 or fewer nucleosides. The immunostimulating oligonucleotide can contain a total of at least 6, about 10 or more, or about 12 or more nucleosides. For example, the immunostimulating oligonucleotide can contain a total of from about 6 to about 30, from about 6 to about 28, from about 6 to about 20, from about 6 to about 16, from about 10 to about 20, from about 10 to about 16, from about 12 to about 28, from about 12 to about 20, or from about 12 to about 16 nucleosides.

In certain embodiments, the immunostimulating oligonucleotide comprises one or more phosphotriesters (e.g., internucleoside phosphotriesters) and/orphosphorothioates (e.g., from about 1 to about 6 or from about 1 to about 4), e.g., at one or both termini (e.g., within the six 5′-terminal nucleosides or the six 3′-terminal nucleosides). The inclusion of one or more internucleoside phosphotriesters and/or phosphorothioates can enhance the stability of the oligonucleotide by reducing the rate of exonuclease-mediated degradation.

In certain embodiments, the immunostimulating oligonucleotide comprises a phosphotriester or a terminal phosphodiester, where the phosphotriester or the terminal phosphodiester comprises a linker bonded to a targeting moiety or a conjugating group and optionally to one or more (e.g., from about 1 to about 6) auxiliary moieties. In certain embodiments, the immunostimulating oligonucleotide comprises only one linker. In certain embodiments, the immunostimulating oligonucleotide comprises only one conjugating group.

The oligonucleotide provided herein can be a hybridized oligonucleotide including a strand and its partial or whole complement. The hybridized oligonucleotides can have at least 6 complementary base pairings (e.g., about 6, about 7, about 8, about 9, about 10, about 11, about 12, about 13, about 14, about 15, about 16, about 17, about 18, about 19, about 20, about 21, about 22, or about 23), up to the total number of the nucleotides present in the included shorter strand. For example, the hybridized portion of the hybridized oligonucleotide can contain about 6, about 7, about 8, about 9, about 10, about 11, about 12, about 13, about 14, about 15, about 16, about 17, about 18, about 19, about 20, about 21, about 22, or about 23 base pairs.

In one aspect, the oligonucleotide in the oligonucleotide-nectin-4 antibody conjugate comprises one or more CpG sites. In some embodiments, the oligonucleotide comprises at least 1, at least 2, or at least 3 CpG sites. In some embodiments, the oligonucleotide is an antisense oligonucleotide. As used herein, a “modified nucleotide” is a nucleotide other than a ribonucleotide (2′-hydroxyl nucleotide). In some embodiments, at least 50%, at least 55%, at least 60%, at least 65%, at least 70%, at least 75%, at least 80%, at least 85%, at least 90%, at least 95%, at least 97%, at least 98%, at least 99%, or 100% of the nucleotides are modified nucleotides. As used herein, modified nucleotides include, but are not limited to, deoxyribonucleotides, nucleotide mimics, abasic nucleotides, 2′-modified nucleotides, 3′ to 3′ linkages (inverted) nucleotides, non-natural base-comprising nucleotides, bridged nucleotides, peptide nucleic acids (PNAs), 2′,3′-seco nucleotide mimics (unlocked nucleobase analogues), locked nucleotides, 3′-O⁻methoxy (2′ internucleoside linked) nucleotides, 2′-F-Arabino nucleotides, 5′-Me, 2′-fluoro nucleotide, morpholino nucleotides, vinyl phosphonate deoxyribonucleotides, vinyl phosphonate containing nucleotides, and cyclopropyl phosphonate containing nucleotides (cPrpN). The 2′-modified nucleotides (i.e. a nucleotide with a group other than a hydroxyl group at the 2′ position of the five-membered sugar ring) include, but are not limited to, 2′-O⁻alkyl nucleotides, 2′-deoxy-2′-halo nucleotides, 2′-deoxy nucleotides, 2′-methoxyethyl (2′-O-2-methoxylethyl) nucleotides, 2′-amino nucleotides, 2′aminoalkyl nucleotides, and 2′-alkyl nucleotides. In some embodiments, modified nucleotide is selected from the group consisting of 5-bromo-2′-O⁻methyluridine, 5-bromo-2′-deoxyuridine, 2′-O⁻methyluridine, 2′-deoxyuridine, 2′-O⁻methylthymidine, 2′-O⁻methylcytidine, 2′-O⁻(2-methoxyethyl)thymidine and 8-oxo-7,8-dihydro-2′-deoxyguanosine. It is not necessary for all positions in a given compound to be uniformly modified. Conversely, more than one modification may be incorporated in a single oligonucleotide or even in a single nucleotide thereof. The oligonucleotides may be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and O-6 substituted purines, (e.g., 2-aminopropyladenine, 5-propynyluracil, or 5-propynylcytosine), 5-methylcytosine (5-Me-C), 5-hydroxymethyl cytosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil (e.g., 5-bromouracil and 5-iodouracil), cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl, 8-oxo and other 8-substituted adenines and guanines, 5-halo (e.g., 5-bromo and 5-iodo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, one or more nucleotides of the oligonucleotide are linked by non-standard linkages or backbones (e.g., modified internucleoside linkages or modified backbones). In some embodiments, a modified internucleoside linkage is a non-phosphate-containing covalent internucleoside linkage. Modified internucleoside linkages or backbones include, but are not limited to, 5′-phosphorothioate groups, chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, the oligonucleotide comprises at least 2, at least 3, at least 4, at least 5, at least 6, at least 7, at least 8, at least 9, at least 10, at least 11, at least 12, at least 13, at least 14, or at least 15 phosphorothioate linkages. In some embodiments, the oligonucleotide comprises at least 2, at least 3, at least 4, at least 5, at least 6, at least 7, at least 8, at least 9, at least 10, at least 11, at least 12, at least 13, at least 14, or at least 15 phosphorodithioate linkages. In some embodiments, the oligonucleotide comprises 1, 2, 3, 4, 5, 6, 7, 8, 9, 10, 11, 12, 13, 14, or 15 phosphorothioate linkages. In some embodiments, the oligonucleotide comprises 1, 2, 3, 4, 5, 6, 7, 8, 9, 10, 11, 12, 13, 14, or 15 phosphorodithioate linkages. In some embodiments, the phosphorothioate internucleoside linkages or phosphorodithioate internucleoside linkages are between the nucleotides at positions 1-3, 2-4, 3-5, 4-6, 4-5, 6-8, 7-9, 8-10, 9-11, 10-12, 11-13, 12-14, 13-15, 14-16, 15-17, 16-18, 17-19, 18-20 or 19-21 from the 5′ end of the oligonucleotide. In some embodiments, the oligonucleotide comprises one or more modified nucleotides and one or more modified internucleoside linkages.

In some embodiments, the oligonucleotide comprises a terminal cap. In some embodiments, the terminal cap is at the 3′ end of the oligonucleotide. In some embodiments, the terminal cap is at the 5′ end of the oligonucleotide. In some embodiments, the terminal cap is at the 5′ end and 3′ end of the oligonucleotide. The term “terminal cap” can also be referred to as “cap,” and has meaning generally accepted in the art. For example, the term refers to a moiety, which can be a chemically modified nucleotide or non-nucleotide that can be incorporated at one or more termini of one or more nucleic acid molecules of the invention. These terminal modifications can protect the nucleic acid molecule from exonuclease degradation, and can help in delivery and/or localization within a cell. In non-limiting examples, the cap includes, but is not limited to a polymer; a ligand; locked nucleic acid (LNA); glyceryl; an abasic ribose residue; inverted deoxy abasic residue; an inverted nucleotide; 4′,5′-methylene nucleotide; 1-(beta-D-erythrofuranosyl) nucleotide; 5′-mercapto moieties; 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide; 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; 5′-5′-inverted nucleotide moiety; 5′-5′-inverted abasic moiety; 5′-phosphoramidate; 5′-phosphorothioate; 1,4-butanediol phosphate; 5′-amino; bridging and/or nonbridging 5′-phosphoramidate; phosphorothioate and/or phosphorodithioate; or bridging or non-bridging methylphosphonate moiety. In some embodiments, the oligonucleotide comprises one or more terminal cap molecules. In some embodiments, [N] is a 3′ terminal cap. In some embodiments, the 3′ terminal cap is O⁻(3-hydroxypropyl)phosphorothioate.

In some embodiments, the oligonucleotide is about 10-30, about 10-15, about 15-20, about 20-25, about 25-30, about 15-25 nucleotides in length. In some embodiments, the oligonucleotide is about 18, 19, 20, 21, 22, 23, 24 or 25 nucleotides in length.

In another aspect, the oligonucleotide of the conjugate comprises:

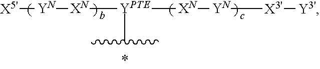

wherein
b and c are each independently an integer from 1 to 25; with the proviso that the sum of b and c is at least 5;
[symbol] indicates the point of attachment of the immunomodulating oligonucleotide P to the rest of the conjugate;
X⁵ is a 5′ terminal nucleoside comprising the structure

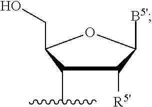

X³ is a 3′ terminal nucleoside comprising the structure

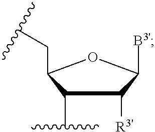

Y^{PTE} is an internucleoside phosphotriester comprising the structure

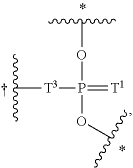

wherein * indicates the points of attachment to the rest of the oligonucleotide and [symbol] † indicates the point of attachment to the linker L, or, if L is absent, [symbol] † indicates the point of attachment to the Q tag peptide Q at the glutamine residue via an amide bond;
Y³ is a terminal phosphotriester comprising the structure

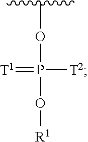

each X^{N} is independently a nucleoside comprising the structure

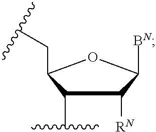

each Y^{N} is independently an internucleoside linker comprising the structure

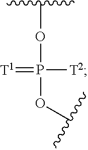

wherein each B^{N} is independently a modified or unmodified nucleobase;
each R^{N} is independently —H or —O⁻C₁₋₄-alkyl, wherein the C₁₋₄-alkyl of the —O⁻C₁₋₄-alkyl is optionally further substituted by —O⁻C₁₋₄-alkyl;
B^{5′} and B^{3′} are independently a modified or unmodified nucleobase;
R^{5′} and R^{3′} are independently —H or —O⁻C₁-C₄-alkyl, wherein the C₁₋₄-alkyl of the —O—C₁₋₄-alkyl is optionally further substituted by —O⁻C₁₋₄-alkyl;
each T₁ is independently O or S;
each T₂ is independently O⁻or S⁻; and
T₃ is a group comprising an oligoethylene glycol moiety; and
R¹ is C₁₋₄-alkylene-hydroxy.

In certain embodiments, the oligonucleotide comprises a nucleotide with a modified nucleobase. In some embodiments, B^{5′} is a modified nucleobase. In other embodiments, B^{3′} is a modified nucleobase. In some embodiments, B^{5′} is an unmodified nucleobase. In other embodiments, B^{3′} is an unmodified nucleobase. In still other embodiments, at least one B^{N} is a modified nucleobase.

In certain embodiments, b is an integer ranging from about 1 to about 15. In certain embodiments, b is an integer of about 1, about 2, about 3, about 4, about 5, about 6, about 7, about 8, about 9, about 10, about 11, about 12, about 13, about 14, or about 15. In certain embodiments, b is an integer of about 3, about 4, about 11, or about 14. In certain embodiments, b is an integer of about 3. In certain embodiments, b is an integer of about 4. In certain embodiments, b is an integer of about 11. In certain embodiments, b is an integer of about 14.

In certain embodiments, c is an integer ranging from about 0 to about 10. In certain embodiments, c is an integer of about 0, about 1, about 2, about 3, about 4, about 5, about 6, about 7, about 8, about 9, or about 10. In certain embodiments, c is an integer of about 0 or about 8. In certain embodiments, c is an integer of about 0. In certain embodiments, c is an integer of about 8.

In certain embodiments, b is an integer of about 3 and c is an integer of about 8. In certain embodiments, b is an integer of about 4 and c is an integer of about 8. In certain embodiments, b is an integer of about 11 and c is an integer of about 0. In certain embodiments, b is an integer of about 14 and c is an integer of about 0.

In certain embodiments, b and c together in total are ranging from about 5 to about 20. In certain embodiments, b and c together in total are ranging from about 5 to about 15. In certain embodiments, b and c together in total are about 5, about 6, about 7, about 8, about 9, about 10, about 11, about 12, about 13, about 14, or about 15. In certain embodiments, b and c together in total are about 8, about 9, about 10, about 11, about 12, about 13, or about 14. In certain embodiments, b and c together in total are about 11. In certain embodiments, b and c together in total are about 12. In certain embodiments, b and c together in total are about 14.

In certain embodiments, each X^{N} is independently a 2′-deoxyribonucleoside or a 2′-modified ribonucleoside. In certain embodiments, each X^{N} is independently 2′-deoxyadenosine (A), 2′-deoxyguanosine (G), 2′-deoxycytidine (C), a 5-halo-2′-deoxycytidine, 2′-deoxythymidine (T), 2′-deoxyuridine (U), a 5-halo-2′-deoxyuridine, a 2′-fluororibonucleoside, a 2′-methoxyribonucleoside, or a 2′-(2-methoxyethoxy)ribonucleoside. In certain embodiments, each X^{N} is independently a 2′-deoxyribonucleoside. In certain embodiments, each X^{N} is independently 2′-deoxyadenosine, 2′-deoxyguanosine, 2′-deoxycytidine, a 5-halo-2′-deoxycytidine, 2′-deoxythymidine, 2′-deoxyuridine, or a 5-halo-2′-deoxyuridine. In certain embodiments, each X^{N} is independently 2′-deoxyadenosine, 2′-deoxyguanosine, 2′-deoxycytidine, 2′-deoxythymidine, 5-bromo-2′-deoxyuridine, or 5-iodo-2′-deoxyuridine.

In certain embodiments, X^{3′} is a 2′-deoxyribonucleoside or a 2′-modified ribonucleoside. In certain embodiments, X^{3′} is a 2′-deoxyribonucleoside. In certain embodiments, X^{3′} is 2′-deoxyadenosine, 2′-deoxyguanosine, 2′-deoxycytidine, a 5-halo-2′-deoxycytidine, 2′-deoxythymidine, 2′-deoxyuridine, a 5-halo-2′-deoxyuridine, a 2′-fluororibonucleoside, a 2′-methoxyribonucleoside, or a 2′-(2-methoxyethoxy)ribonucleoside. In certain embodiments, X^{3′} is 2′-deoxyadenosine, 2′-deoxyguanosine, 2′-deoxycytidine, a 5-halo-2′-deoxycytidine, 2′-deoxythymidine, 2′-deoxyuridine, or a 5-halo-2′-deoxyuridine. In certain embodiments, X^{3′} is 2′-deoxythymidine. In certain embodiments, X^{3′} is a 2′-deoxyribonucleoside with a substituted pyrimidine base. In certain embodiments, X^{3′} is a 2′-deoxyribonucleoside with a 5-substituted pyrimidine base. In certain embodiments, X^{3′} is 2′-deoxythymidine, a 5-halo-2′-deoxycytidine, or a 5-halo-2′-deoxyuridine. In certain embodiments, X^{3′} is 2′-deoxythymidine, 5-bromo-2′-deoxycytidine, 5-iodo-2′-deoxycytidine, 5-bromo-2′-deoxyuridine, or 5-iodo-2′-deoxyuridine. In certain embodiments, X^{3′} is 2′-deoxythymidine, 5-bromo-2′-deoxyuridine, or 5-iodo-2′-deoxyuridine. In certain embodiments, X^{3′} is a terminal nucleotide comprising a 3′ capping group. In certain embodiments, the 3′ capping group is a terminal phosphoester. In certain embodiments, the 3′ capping group is 3-hydroxyl-propylphosphoryl (i.e., —P(O₂)—OCH₂CH₂CH₂OH).

In certain embodiments, X^{5′} is a 2′-deoxyribonucleoside or a 2′-modified ribonucleoside. In certain embodiments, X^{5′} is a 2′-deoxyribonucleoside. In certain embodiments, X^{5′} is 2′-deoxyadenosine, 2′-deoxyguanosine, 2′-deoxycytidine, a 5-halo-2′-deoxycytidine, 2′-deoxythymidine, 2′-deoxyuridine, a 5-halo-2′-deoxyuridine, a 2′-fluororibonucleoside, a 2′-methoxyribonucleoside, or a 2′-(2-methoxyethoxy)ribonucleoside. In certain embodiments, X^{5′} is 2′-deoxyadenosine, 2′-deoxyguanosine, 2′-deoxycytidine, a 5-halo-2′-deoxycytidine, 2′-deoxythymidine, 2′-deoxyuridine, or a 5-halo-2′-deoxyuridine. In certain embodiments, X^{5′} is a 2′-deoxyribonucleoside with a substituted pyrimidine base. In certain embodiments, X^{5′} is a 2′-deoxyribonucleoside with a 5-substituted pyrimidine base. In certain embodiments, X^{5′} is 2′-deoxythymidine, a 5-halo-2′-deoxycytidine, or a 5-halo-2′-deoxyuridine. In certain embodiments, X^{5′} is a 5-halo-2′-deoxycytidine. In some embodiments, X^{5′} is a 2′-deoxyuridine, a 5-halo-2′-deoxyuridine, 2′-methoxyuridine, or a 5-halo-2′-methoxyuridine. In certain embodiments, X^{5′} is a 5-halo-2′-deoxyuridine. In certain other embodiments, X^{5′} is a 2′-deoxyuridine. In certain embodiments, X^{5′} is a 5-halo-2′-methoxyuridine. In certain other embodiments, X^{5′} is a 2′-methoxyuridine. In certain embodiments, X^{5′} is 2′-deoxythymidine, 5-bromo-2′-deoxycytidine, 5-iodo-2′-deoxycytidine, 5-bromo-2′-deoxyuridine, or 5-iodo-2′-deoxyuridine. In certain embodiments, X^{5′} is 2′-deoxythymidine, 5-bromo-2′-deoxyuridine, or 5-iodo-2′-deoxyuridine. In certain embodiments, X^{5′} is 5-bromo-2′-deoxyuridine. In certain embodiments, X^{5′} is 5-iodo-2′-deoxyuridine. In certain embodiments, X^{5′} has a 3′-phosphorothioate group. In certain embodiments, X^{5′} has a 3′-phosphorothioate group with a chirality of Rp. In certain embodiments, X^{5′} has a 3′-phosphorothioate group with a chirality of Sp.

In certain embodiments, Y^{PTE} is an internucleoside phosphothiotriester.

In some embodiments, Y^{PTE} is

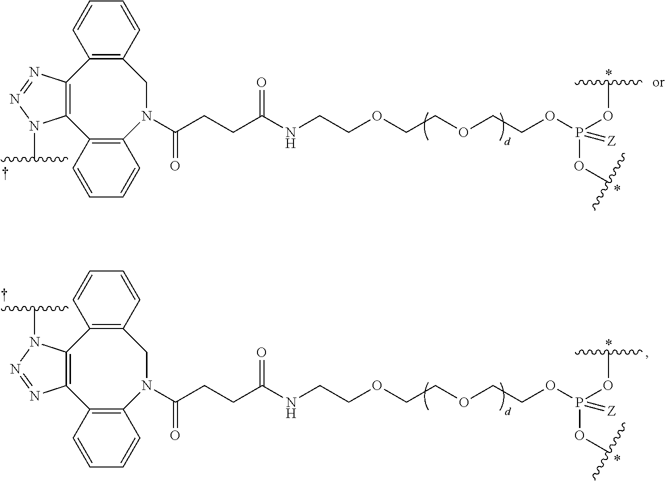

wherein Z is O or S; d is an integer ranging from about 0 to about 50; the two [symbol] * on the right side of the structure indicate the points of attachment to the oligonucleotide P; and the [symbol] † on the left side of the structure indicates the point of attachment to the rest of the conjugate. In certain embodiments, Z is O. In certain embodiments, Z is S. In certain embodiments, d is an integer ranging from about 0 to about 10. In certain embodiments, d is an integer ranging from about 0 to about 5. In certain embodiments, d is an integer of about 0, about 1, about 2, about 3, about 4, or about 5. In certain embodiments, d is an integer of about 0, about 1, or about 3.

In some embodiments, Y^{PTE} is

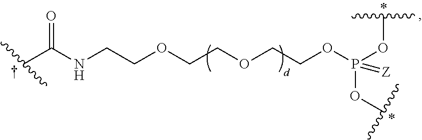

wherein Z is O or S; d is an integer ranging from about 0 to about 50; the two [symbol] * on the right side of the structure indicate the points of attachment to the oligonucleotide P; and the [symbol] † on the left side of the structure indicates the point of attachment to the rest of the conjugate. In certain embodiments, Z is O. In certain embodiments, Z is S. In certain embodiments, d is an integer ranging from about 0 to about 10. In certain embodiments, d is an integer ranging from about 0 to about 5. In certain embodiments, d is an integer of about 0, about 1, about 2, about 3, about 4, or about 5. In certain embodiments, d is an integer of about 0, about 1, or about 3.

In certain embodiments, the oligonucleotide comprises one additional internucleoside phosphotriester. In one embodiment, the additional internucleoside phosphotriester is a C₁₋₆ alkylphosphotriester. In another embodiment, the additional internucleoside phosphotriester is ethylphosphotriester.

In certain embodiments, the oligonucleotide comprises one 5-halo-2′-deoxyuridine. In one embodiment, the 5-halo-2′-deoxyuridine is 5-fluoro-2′-deoxyuridine, 5-bromo-2′-deoxyuridine, or 5-iodo-2′-deoxyuridine. In another embodiment, the 5-halo-2′-deoxyuridine is 5-bromo-2′-deoxyuridine or 5-iodo-2′-deoxyuridine. In yet another embodiment, the 5-halo-2′-deoxyuridine is 5-fluoro-2′-deoxyuridine. In yet another embodiment, the 5-halo-2′-deoxyuridine is 5-bromo-2′-deoxyuridine. In still another embodiment, the 5-halo-2′-deoxyuridine is 5-iodo-2′-deoxyuridine.

In certain embodiments, the oligonucleotide comprises three or more 2′-deoxycytidines. In certain embodiments, the oligonucleotide comprises three 2′-deoxycytidines.

In certain embodiments, the oligonucleotide comprises four or more 2′-deoxyguanosines. In certain embodiments, the oligonucleotide comprises four 2′-deoxyguanosines.

In certain embodiments, the oligonucleotide comprises three 2′-deoxycytidines and four 2′-deoxyguanosines. In certain embodiments, the oligonucleotide comprises one, two, or three CG dinucleotides. In certain embodiments, the oligonucleotide comprises three CG dinucleotides.

In certain embodiments, the oligonucleotide comprises three or more 2′-deoxythymidines. In certain embodiments, the oligonucleotide comprises three, four, five, six, seven, or eight 2′-deoxythymidines. In certain embodiments, the oligonucleotide comprises three, four, five, or eight 2′-deoxythymidines.

In certain embodiments, the oligonucleotide does not comprise a 2′-deoxyadenosine. In certain embodiments, the oligonucleotide comprises one or two 2′-deoxyadenosines.

In certain embodiments, the oligonucleotide has a length ranging from about 5 to about 20 or from about 6 to about 15. In certain embodiments, the oligonucleotide has a length of about 6, about 7, about 8, about 9, about 10, about 11, about 12, about 13, about 14, or about 15. In certain embodiments, the oligonucleotide has a length of about 10, about 11, about 12, about 13, about 14, or about 15.

In certain embodiments, the oligonucleotide comprises one or more internucleoside phosphorothioates. In certain embodiments, all the internucleoside phosphoesters in the oligonucleotide are internucleoside phosphorothioates. In certain embodiments, the oligonucleotide comprises one or more chiral internucleoside phosphorothioates.

In certain embodiments, the oligonucleotides comprising a sequence of N¹N²CGN³CG(T)_{X}GN⁴CGN⁵T (SEQ ID NO:174), or a stereoisomer, a mixture of two or more diastereomers, a tautomer, or a mixture of two or more tautomers thereof; or a pharmaceutically acceptable salt, solvate, or hydrate thereof are as described in, for example, WO2018/189382 A1.

In one embodiment, the oligonucleotide comprises a sequence of N¹N²CGN³CG(T)_{X}GN⁴CGN⁵T (SEQ ID NO:174), or a stereoisomer, a mixture of two or more diastereomers, a tautomer, or a mixture of two or more tautomers thereof; or a pharmaceutically acceptable salt, solvate, or hydrate thereof; wherein:
x is an integer ranging from about 1 to about 4;
N¹ is absent or 2′-deoxythymidine;
N² is a 2′-deoxyribonucleotide with a modified nucleobase;
N³ is 2′-deoxyadenosine or 2′-deoxythymidine, each optionally comprising a 3′-phosphotriester;
N⁴ is 2′-deoxyadenosine or 2′-deoxythymidine;
N⁵ is 2′-deoxythymidine optionally comprising a 3′-phosphotriester; and
C is 2′-deoxycytidine and G is 2′-deoxyguanosine.

In certain embodiments, in N¹N²CGN³CG(T)_{X}GN⁴CGN⁵T (SEQ ID NO:174), x is an integer of about 1, about 2, about 3, or about 4. In certain embodiments, in N¹N²CGN³CG(T)_{X}GN⁴CGN⁵T (SEQ ID NO:174), x is an integer of about 1. In certain embodiments, in N¹N²CGN³CG(T)_{X}GN⁴CGN⁵T (SEQ ID NO:174), x is an integer of about 4.

In certain embodiments, in N¹N²CGN³CG(T)_{X}GN⁴CGN⁵T (SEQ ID NO:174), N¹ is absent. In certain embodiments, in N¹N²CGN³CG(T)_{X}GN⁴CGN⁵T (SEQ ID NO:174), N¹ is 2′-deoxythymidine.

In certain embodiments, in N¹N²CGN³CG(T)_{X}GN⁴CGN⁵T (SEQ ID NO:174), N² is a 2′-deoxyribonucleotide with a substituted pyrimidine base. In certain embodiments, in N¹N²CGN³CG(T)_{X}GN⁴CGN⁵T (SEQ ID NO:174), N² is a 2′-deoxyribonucleotide with a 5-substituted pyrimidine base. In certain embodiments, in N¹N²CGN³CG(T)_{X}GN⁴CGN⁵T (SEQ ID NO:174), N² is a 5-halo-2′-deoxycytidine or a 5-halo-2′-deoxyuridine. In certain embodiments, in N¹N²CGN³CG(T)_{X}GN⁴CGN⁵T (SEQ ID NO:174), N² is 5-bromo-2′-deoxyuridine or 5-iodo-2′-deoxyuridine.

In certain embodiments, in N¹N²CGN³CG(T)_{X}GN⁴CGN⁵T (SEQ ID NO:174), N³ is 2′-deoxyadenosine comprising a 3′-phosphotriester. In certain embodiments, in N¹N²CGN³CG(T)_{X}GN⁴CGN⁵T (SEQ ID NO:174), N³ is 2′-deoxythymidine. In certain embodiments, in N¹N²CGN³CG(T)_{X}GN⁴CGN⁵T (SEQ ID NO:174), N³ is 2′-deoxythymidine comprising a 3′-phosphotriester.

In certain embodiments, in N¹N²CGN³CG(T)_{X}GN⁴CGN⁵T (SEQ ID NO:174), N⁴ is 2′-deoxyadenosine. In certain embodiments, in N¹N²CGN³CG(T)_{X}GN⁴CGN⁵T (SEQ ID NO:174), N⁴ is 2′-deoxythymidine.

In certain embodiments, in N¹N²CGN³CG(T)_{X}GN⁴CGN⁵T (SEQ ID NO:174), N⁵ is 2′-deoxythymidine. In certain embodiments, in N¹N²CGN³CG(T)_{X}GN⁴CGN⁵T (SEQ ID NO:174), N⁵ is 2′-deoxythymidine comprising a 3′-phosphotriester.

In certain embodiments, the oligonucleotide of N¹N²CGN³CG(T)_{X}GN⁴CGN⁵T (SEQ ID NO:174) comprises one or more internucleoside phosphorothioates or phosphorotdithioates. In certain embodiments, the oligonucleotide of N¹N²CGN³CG(T)_{X}GN⁴CGN⁵T (SEQ ID NO:174) comprises at least one chiral internucleoside phosphorothioate or phosphorotdithioates. In certain embodiments, the oligonucleotide of N¹N²CGN³CG(T)_{X}GN⁴CGN⁵T (SEQ ID NO:174) comprises at least one chiral phosphorotdithioates. In certain embodiments, the oligonucleotide of N¹N²CGN³CG(T)_{X}GN⁴CGN⁵T (SEQ ID NO:174) is an oligonucleotide sequence as described in, for example, WO2018/189382 A1.

In certain embodiments, the oligonucleotide is an immunostimulating oligonucleotide. In certain embodiments, the oligonucleotide provided herein functions as a PAMS. In certain embodiments, the oligonucleotide provided herein activates innate immune response or stimulates the adaptive immune response by triggering TLR9 signaling. In certain embodiments, the oligonucleotide provided herein is a TLR9 agonist.

In certain embodiments, the oligonucleotide is a CpG oligonucleotide, comprising a modification including 5-halouridine or 5-alkynyluridine, or a truncated version thereof (e.g., those comprising a total of about 6 to about 16 nucleosides). In certain embodiments, the truncated oligonucleotide provided herein comprises a truncated oligonucleotide sequence, from which one or more 3′-terminal nucleotides are eliminated or one or more of the intra-sequence nucleotides excised).

In certain embodiments, the oligonucleotide comprises at least one immunostimulating sequence (ISS). In certain embodiments, the oligonucleotide provided herein comprises about 1, about 2, about 3, or about 4 ISS. The ISS in immunostimulating oligonucleotides is dependent on the targeted organism. The common feature of the ISS used in the oligonucleotide provided herein is the cytidine-p-guanosine sequence, in which p is an internucleoside phosphodiester (e.g., phosphate or phosphorothioate) or an internucleoside phosphotriester. In certain embodiments, cytidine and guanosine in the ISS each independently comprises 2′-deoxyribose. In certain embodiments, the oligonucleotide provided herein comprises about 1, about 2, or about 3 human ISSs. In certain embodiments, the human ISS is CG or NCG, where N is uridine, cytidine, or thymidine, or a modified uridine or cytidine; and G is guanosine or a modified guanosine. In certain embodiments, the modified uridine or cytidine is a 5-halouridine (e.g., 5-iodouridine or 5-bromouridine), a 5-alkynyluridine (e.g., 5-ethynyluridine or 5-propynyluridine), 5-heteroaryluridine, or 5-halocytidine. In certain embodiments, the modified guanosine is 7-deazaguanosine. In certain embodiments, the human ISS is NCG, in one embodiment, N is 5-halouridine. In certain embodiments, the human ISS is UCG, in one embodiment, U is 5-alkynyluridine, and in another embodiment, U is 5-ethynyluridine. In certain embodiments, the oligonucleotide provided herein targeting humans comprises an ISS within four contiguous nucleotides that include a 5′-terminal nucleotide. In certain embodiments, the oligonucleotide provided herein targeting humans comprises a 5′-terminal ISS. In certain embodiments, the oligonucleotide provided herein comprises a murine ISS. In certain embodiments, the murine ISS is a hexameric nucleotide sequence: Pu—Pu-CG-Py-Py (SEQ ID NO:498), where each Pu is independently a purine nucleotide, and each Py is independently a pyrimidine nucleotide.

In certain embodiments, the 5′-flanking nucleotides relative to CpG in the oligonucleotide provided herein does not contain 2′-alkoxyriboses. In certain embodiments, the 5′-flanking nucleotides relative to CpG in the oligonucleotide provided herein comprises only 2′-deoxyriboses as sugars.

In certain embodiments, the oligonucleotide has (1) a high content of phosphorothioates or phosphorodithioates (e.g., at least 50%, at least 60%, at least 70%, or at least 80% of nucleosides may be linked by phosphorothioates or phosphorodithioates); (2) absence of poly-G tails; (3) nucleosides in the oligonucleotide comprises 2′-deoxyriboses or 2′-modified riboses (e.g., 2′-halo (e.g., 2′-fluoro, 2′-bromo, or 2′-iodo) or optionally substituted 2′-alkoxy (e.g., 2′-methoxy)); and/or (4) the inclusion of 5′-terminal ISS that is NCG, in which N is uridine, cytidine, or thymidine, or a modified uridine or cytidine, and G is guanosine or a modified guanosine.

In certain embodiments, the oligonucleotide suppresses the adaptive immune response by reducing activation of TLR9 signaling (e.g., through TLR9 antagonism). In certain embodiments, the immunosuppressive oligonucleotide provided herein comprises at least two 2′-alkoxynucleotides that are 5′-flanking relative to CpG as described by the formula of N¹—N²—CG, where N¹ and N² are each independently a nucleotide containing 2′-alkoxyribose (e.g., 2′-methoxyribose). In some embodiments, the immunosuppressive oligonucleotides are methylated.

In some embodiments, the oligonucleotide comprises the structure:

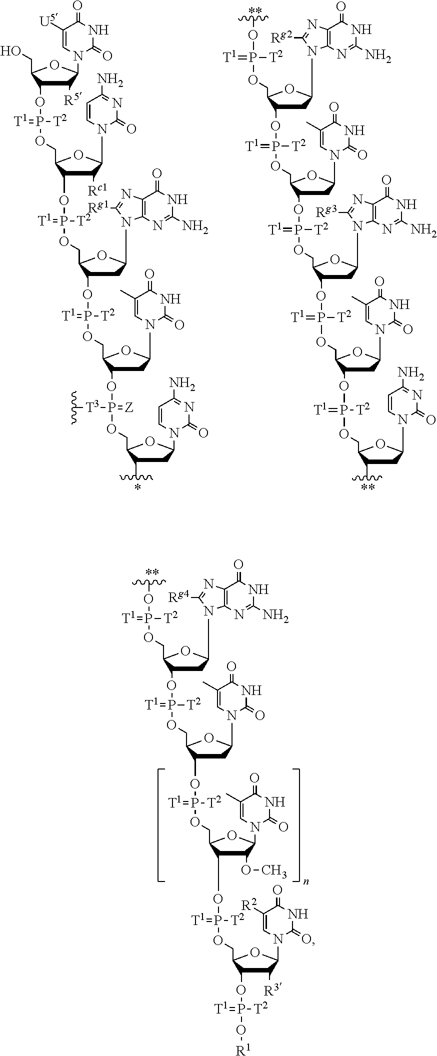

wherein
[symbol] * and [symbol] ** indicate the points of attachment within the oligonucleotide;
each T¹ is independently O or S;
each T² is O⁻ or S⁻;
T³ is a group

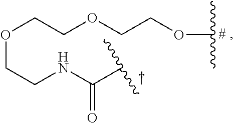 [SMILES: *OCCOCCOCCNC(*)=O]

wherein [symbol] † indicates the point of attachment to L and wherein [symbol] # indicates the point of attachment to the rest of the oligonucleotide;
Z is 0 or S;
U^{5′} is —H or halogen;
R^{5′} is —H or methoxy;
R^{c1} is —H or methoxy;
R^{g1}, R^{g2}, R^{g3}, and R⁹⁴ are H or oxo, wherein if one of R^{g1}, R^{g2}, R^{g3}, and R⁹⁴ is oxo, then the carbon to which the oxo is attached has a single bond to the ring nitrogen at the 7-position;
R^{3′} is methoxy;
R¹ is C₁₋₄-alkylene-hydroxy;
R² is —H or methyl; and
n is an integer from 0 to 2.

In other embodiments, the oligonucleotide comprises the structure:

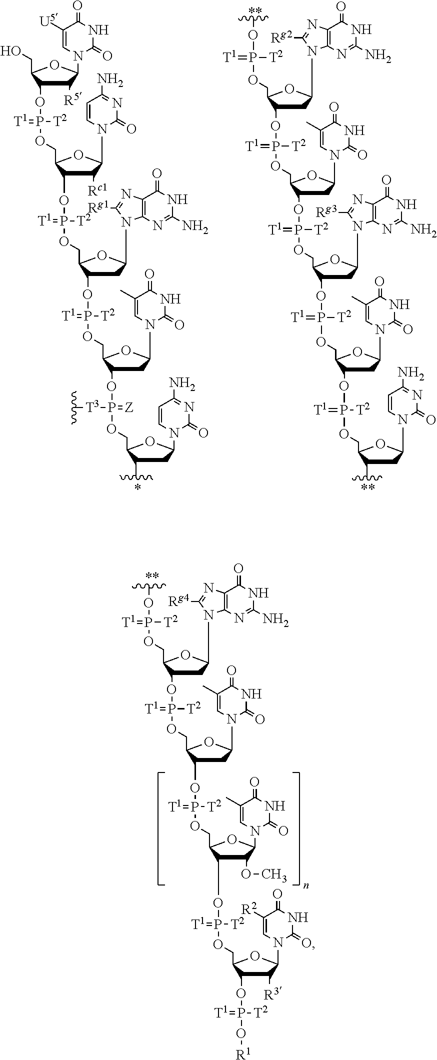

wherein
[symbol] * and [symbol] ** indicate the points of attachment within the oligonucleotide;
each T¹ is independently O or S;
each T² is O⁻or S⁻;
T³ is a group

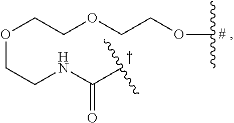 [SMILES: *OCCOCCOCCNC(*)=O]

wherein [symbol] † indicates the point of attachment to L and wherein [symbol] # indicates the point of attachment to the rest of the oligonucleotide;
Z is O or S;
R^{5′} is —H or methoxy;
R^{c1} is —H or methoxy;
R^{g1}, R^{g2}, R^{g3}, and R⁹⁴ are H or oxo, wherein if one of R^{g1}, R^{g2}, R^{g3}, and R⁹⁴ is oxo, then the carbon to which the oxo is attached has a single bond to the ring nitrogen at the 7-position;
R^{3′} is methoxy;
R¹ is C₁₋₄-alkylene-hydroxy;
R² is —H or methyl; and
n is an integer from 0 to 2.

In still other embodiments, the oligonucleotide comprises the structure:

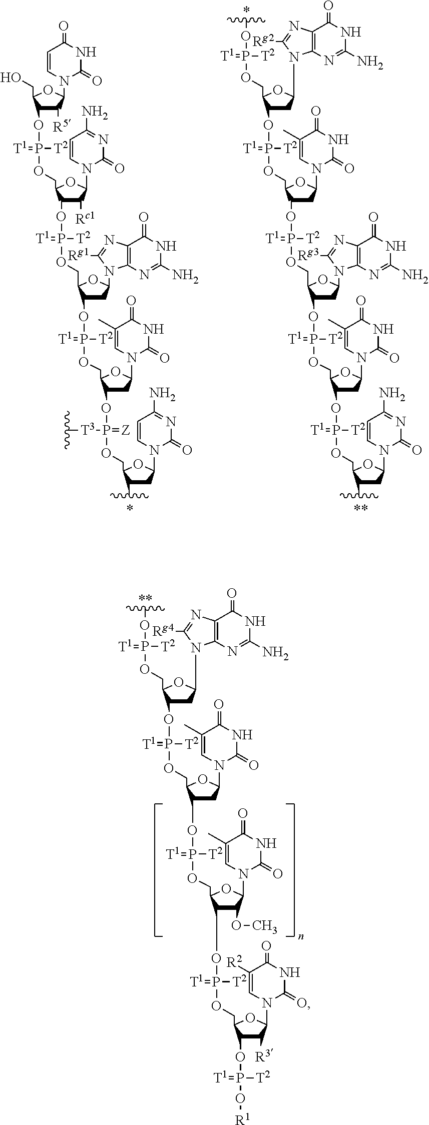

wherein
[symbol] * and [symbol] ** indicate the points of attachment within the oligonucleotide;
each T¹ is independently O or S;
each T² is O⁻or S⁻;
T³ is a group

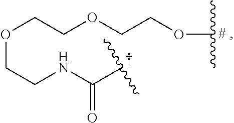 [SMILES: *OCCOCCOCCNC(*)=O]

wherein [symbol] † indicates the point of attachment to L and wherein [symbol] # indicates the point of attachment to the rest of the oligonucleotide;
Z is O or S;
R^{5′} is —H or methoxy;
R^{c1} is —H or methoxy;
R^{g1}, R^{g2}, R^{g3}, and R⁹⁴ are H or oxo, wherein if one of R^{g1}, R^{g2}, R^{g3}, and R⁹⁴ is oxo, then the carbon to which the oxo is attached has a single bond to the ring nitrogen at the 7-position;
R^{3′} is methoxy;
R¹ is C₁₋₄-alkylene-hydroxy;
R² is —H or methyl; and
n is an integer from 0 to 2.

In some embodiments, the oligonucleotide comprises one or more of unmodified sequences differing by 0, 1, 2 or 3 nucleobases from the sequences shown in Table 1. In some embodiments, the oligonucleotide comprises one or more of modified sequences differing by 0, 1, 2 or 3 nucleobases from the sequences shown in Table 2.

TABLE 1
Unmodified Oligonucleotides

| | Unmodified Oligonucleotide Sequence |
|---|---|
| SEQ ID NO. | (5′→3′) |
| 1 | tucgtcgtgacgtt |
| 2 | ucgtcgtgtcgtt |
| 129 | tcgtcgttttgtcgttttgtcgtt |

TABLE 2
Modified Oligonucleotides

| SEQ ID | Modified Oligonucleotide Sequence | |
|---|---|---|
| NO. | (5′→3′) | Cmpd # |
| 3 | uscsgstscsgstsgstscsgstsT-c3 | 1.1b |
| 4 | uscsgstscsgstsgstscsgstst-c3 | 2.1b |
| 5 | uscsgstscsgstsgstscsgstst-c3 | 2.2b |
| 6 | uscsgstscsgstsgstscsgstst-c3 | 2.3b |
| 7 | uscsgstscsgstsgstscsgstst-c3 | 2.4b |
| 8 | uscsgstscsgstsgstscsgstst-c3 | 3.1b |
| 9 | uscsgstscsgstsgstscsgststst-c3 | 3.2b |
| 10 | uscsgstscsgstsgstscsgstststst-c3 | 3.3b |
| 11 | uscsgstscsgstsgstscsgstst-c3 | 4.1b |
| 12 | uscsgstscsgstsgstscsgststst-c3 | 4.2b |
| 13 | uscsgstscsgstsgstscsgststst-c3 | 4.3b |
| 14 | uscs2gstscsgstsgstscsgstst-c3 | 5.1a |
| 15 | uscsgs2tscsgstsgstscsgstst-c3 | 5.2a |
| 16 | uscsgstscs2gstsgstscsgstst-c3 | 5.3a |
| 17 | uscsgstscsgs2tsgstscsgstst-c3 | 5.4a |
| 18 | uscsgstscsgsts2gstscsgstst-c3 | 5.5a |
| 19 | uscsgstscsgstsgs2tscsgstst-c3 | 5.6a |
| 20 | uscsgstscsgstsgsts2csgstst-c3 | 5.7a |
| 21 | uscsgstscsgstsgstscs2gstst-c3 | 5.8a |
| 22 | uscsgstscsgstsgstscsgs2tst-c3 | 5.9a |
| 23 | uscsgstscsgstsgstscsgsts2t-c3 | 5.10a |
| 24 | uscsgstscsgstsgstscsgstsus2-c3 | 5.11a |
| 25 | uscs2gstscsgstsgstscsgstsus2-c3 | 5.12a |
| 26 | uscsgs2tscsgstsgstscsgststst-c3 | 6.1b |
| 27 | uscsgstscsgstsgsts2csgststst-c3 | 6.2b |
| 28 | uscsgs2tscsgstsgsts2csgststst-c3 | 6.3b |
| 29 | uscs2gstscsgstsgstscsgststst-c3 | 7.1b |
| 30 | uscsgstscsgstsgs2tscsgststst-c3 | 7.2b |
| 31 | uscs2gstscsgstsgs2tscsgststst-c3 | 7.3b |
| 32 | uscsgstscs2gstsgstscsgststst-c3 | 7.4b |
| 33 | uscsgstscsgs2tsgstscsgststst-c3 | 7.5b |
| 34 | uscsgstscsgsts2gstscsgststst-c3 | 7.6b |
| 35 | uscsgstscsgstsgsts2csgststst-c3 | 7.7b |
| 36 | uscs2gstscsgstsgsts2csgststst-c3 | 7.8b |
| 37 | uscsgs2tscsgstsgstscsgststst-c3 | 7.9b |
| 38 | uscsgstscsgstsgstscs2gststst-c3 | 7.10b |
| 130 | uscsgstscsgstsgstscsgstsT-c3 | 8.1b |
| 131 | uscsgstscsgstsgstscsgstst-c3 | 9.1b |
| 132 | uscsgstscsgstsgstscsgstst-c3 | 9.2b |
| 133 | uscsgstscsgstsgstscsgstst-c3 | 9.3b |
| 134 | uscsgstscsgstsgstscsgstst-c3 | 9.4b |
| 135 | uscsgstscsgstsgstscsgstst-c3 | 10.1b |
| 136 | uscsgstscsgstsgstscsgststst-c3 | 10.2b |
| 137 | uscsgstscsgstsgstscsgstststst-c3 | 10.3b |
| 138 | uscsgstscsgstsgstscsgstst-c3 | 12.1b |
| 139 | uscsgstscsgstsgstscsgststst-c3 | 12.2b |
| 140 | uscsgstscsgstsgstscsgststst-c3 | 12.3b |
| 141 | uscs2gstscsgstsgstscsgstst-c3 | 13.1a |
| 142 | uscsgs2tscsgstsgstscsgstst-c3 | 13.2a |
| 143 | uscsgstscs2gstsgstscsgstst-c3 | 13.3a |
| 144 | uscsgstscsgs2tsgstscsgstst-c3 | 13.4a |
| 145 | uscsgstscsgsts2gstscsgstst-c3 | 13.5a |
| 146 | uscsgstscsgstsgs2tscsgstst-c3 | 13.6a |
| 147 | uscsgstscsgstsgsts2csgstst-c3 | 13.7a |
| 148 | uscsgstscsgstsgstscs2gstst-c3 | 13.8a |
| 149 | uscsgstscsgstsgstscsgs2tst-c3 | 13.9a |
| 150 | uscsgstscsgstsgstscsgsts2t-c3 | 13.10a |
| 151 | uscsgstscsgstsgstscsgstsus2-c3 | 13.11a |
| 152 | uscs2gstscsgstsgstscsgstsus2-c3 | 13.12a |
| 153 | uscsgs2tscsgstsgstscsgststst-c3 | 14.1b |
| 154 | uscsgstscsgstsgsts2csgststst-c3 | 14.2b |
| 155 | uscsgs2tscsgstsgsts2csgststst-c3 | 14.3b |
| 156 | uscs2gstscsgstsgstscsgststst-c3 | 15.1b |
| 157 | uscsgstscsgstsgs2tscsgststst-c3 | 15.2b |
| 158 | uscs2gstscsgstsgs2tscsgststst-c3 | 15.3b |
| 159 | uscsgstscs2gstsgstscsgststst-c3 | 15.4b |
| 160 | uscsgstscsgs2tsgstscsgststst-c3 | 15.5b |
| 161 | uscsgstscsgsts2gstscsgststst-c3 | 15.6b |
| 162 | uscsgstscsgstsgsts2csgststst-c3 | 15.7a |
| 163 | uscsgstscsgstsgsts2csgststst-c3 | 15.7b |
| 164 | uscs2gstscsgstsgsts2csgststst-c3 | 15.8b |
| 165 | uscsgs2tscsgstsgstscsgststst-c3 | 15.9b |
| 166 | uscsgstscsgstsgstscs2gststst-c3 | 15.10b |

*u: 5-Bromo-2′-deoxyuridine
g: 8-oxo-7,8-dihydro-2′-deoxyguanosine
u: 5-Bromo 2′-OMe uridine
c: 2′-OMe— Cytidine
t: 2′-OMe— Thymidine
u: 2′-OMe— Uridine
u: 2′-deoxyuridine
T: 2′-OMOE thymidine
ts: phosphotriester linker-PEG₂₄—NH₂ 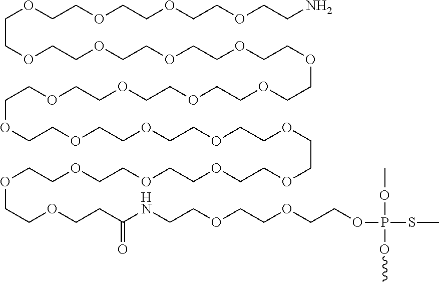
following thymidine;
ts: 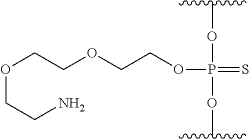 [SMILES: *OP(=S)(O*)OCCOCCOCCN]
phosphotriester linker following thymidine;
Lower case: 2′-deoxy nucleotide
s: phosphorothioate linkage
s2: phosphorodithioate linkage
c3:
s2-c3:

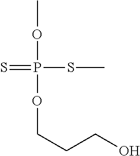

In some embodiments, the oligonucleotide is functionalized with a chemical tag for attachment to the linking moiety. In some embodiments, the chemical tag is attached to an inter-nucleoside linkage of the oligonucleotide. In some embodiments, the chemical tag is attached to a 5′ inter-nucleoside linkage. In some embodiments, the chemical tag is attached to a 3′ inter-nucleoside linkage. In some embodiments, the inter-nucleoside linkage is a phosphorothioate linkage. In some embodiments, the inter-nucleoside linkage is a phosphorodithioate linkage. In some embodiments, the chemical tag is closer to the 5′ end than the 3′ end of the oligonucleotide. In some embodiments, the chemical tag is attached to a nucleobase.

In some embodiments, the immunomodulating oligonucleotide comprises the structure of formula (C),

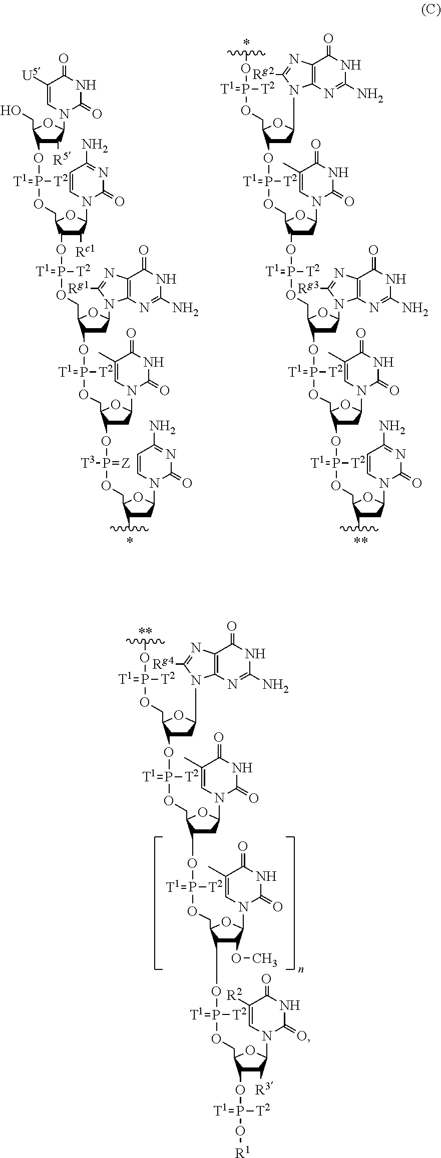

wherein
[symbol] * and [symbol] ** indicate the points of attachment within the oligonucleotide;
each T¹ is independently O or S;
each T² is S⁻;
T³ is a group

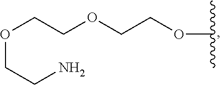 [SMILES: *OCCOCCOCCN]

wherein [symbol] indicates the point of attachment to the rest of the oligonucleotide;
Z is O or S;
U^{5′} is —H or halogen;
R^{5′} is —H or methoxy;
R^{c1} is —H or methoxy;
R^{g1}, R^{g2}, R^{g3}, and R⁹⁴ are H or oxo, optionally wherein at least one of R^{g1}, R^{g2}, R^{g3}, and R⁹⁴ is oxo, and wherein if one of R^{g1}, R^{g2}, R^{g3}, and R⁹⁴ is oxo, then the carbon to which the oxo is attached has a single bond to the ring nitrogen at the 7-position;
R^{3′} is methoxy or 2-methoxyethoxy;
R¹ is —(CH₂)₃—OH;
R² is —H or methyl; and
n is an integer from 0 to 2, or a pharmaceutically acceptable salt thereof.

In some embodiments of the present aspect, U^{5′} is —H In other embodiments, U^{5′} is halogen. In certain embodiments, U^{5′} is iodo or bromo. In some embodiments of the present aspect, the immunomodulatory oligonucleotide of formula (C) is an immunomodulatory oligonucleotide of formula (C′). In other embodiments of the present aspect, the immunomodulatory oligonucleotide of formula (C) is an immunomodulatory oligonucleotide of formula (C″).

In some embodiments the immunomodulatory oligonucleotide comprises the structure of formula (C′),

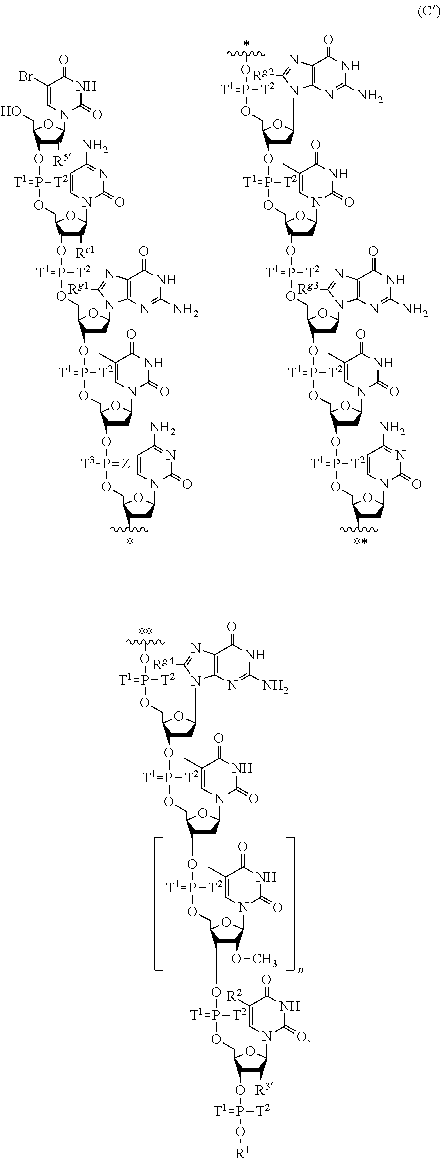

wherein
[symbol] * and [symbol] ** indicate the points of attachment within the oligonucleotide;
each T¹ is independently O or S;
each T² is S⁻;
T³ is a group

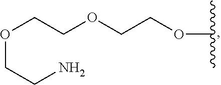 [SMILES: *OCCOCCOCCN]

wherein [symbol] indicates the point of attachment to the rest of the oligonucleotide; [symbol]
Z is O or S;
R^{5′} is —H or methoxy;
R^{c1} is —H or methoxy;
R^{g1}, R^{g2}, R^{g3}, and R⁹⁴ are H or oxo, optionally wherein at least one of R^{g1}, R^{g2}, R^{g3}, and R⁹⁴ is oxo and wherein if one of R^{g1}, R^{g2}, R^{g3}, and R⁹⁴ is oxo then the carbon to which the oxo is attached has a single bond to the ring nitrogen at the 7-position;
R^{3′} is methoxy or 2-methoxyethoxy;
R¹ is —(CH₂)₃—OH;
R² is —H or methyl; and
n is an integer from 0 to 2,
   or a pharmaceutically acceptable salt thereof.

In some embodiments, the immunomodulatory oligonucleotide comprises the structure of formula (C″),

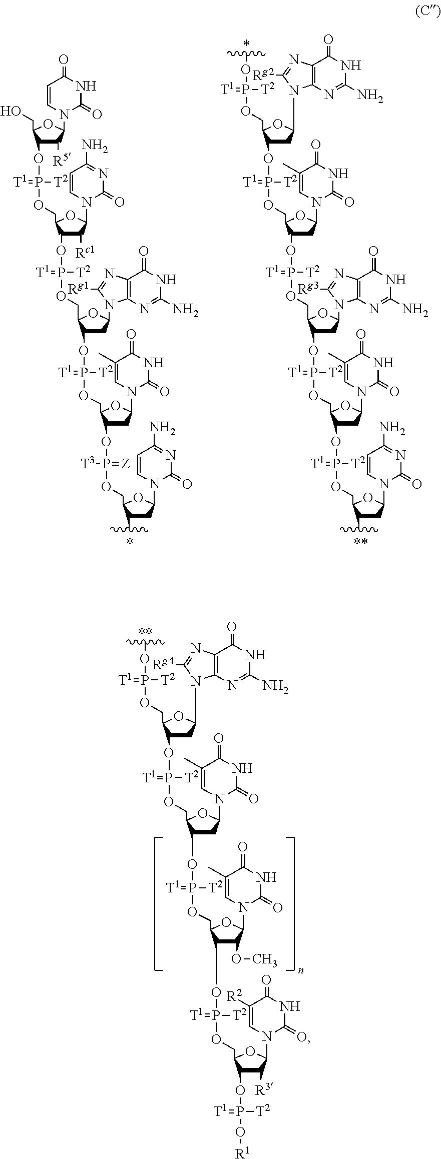

wherein
[symbol] * and [symbol] ** indicate the points of attachment within the oligonucleotide;
each T¹ is independently O or S;
each T² is S⁻;
T³ is a group

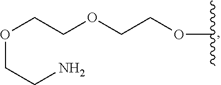 [SMILES: *OCCOCCOCCN]

wherein [symbol] indicates the point of attachment to the rest of the oligonucleotide;
Z is O or S;
R^{5′} is —H or methoxy;
R^{c1} is —H or methoxy;
R^{g1}, R^{g2}, R^{g3}, and R⁹⁴ are H or oxo, optionally wherein at least one of R^{g1}, R^{g2}, R^{g3}, and R⁹⁴ is oxo and wherein if one of R^{g1}, R^{g2}, R^{g3}, and R⁹⁴ is oxo then the carbon to which the oxo is attached has a single bond to the ring nitrogen at the 7-position;
R^{3′} is methoxy or 2-methoxyethoxy;
R¹ is —(CH₂)₃—OH;
R² is —H or methyl; and
n is an integer from 0 to 2,
   or a pharmaceutically acceptable salt thereof.

In some embodiments, the immunomodulatory oligonucleotide comprises the structure of formula (C′),

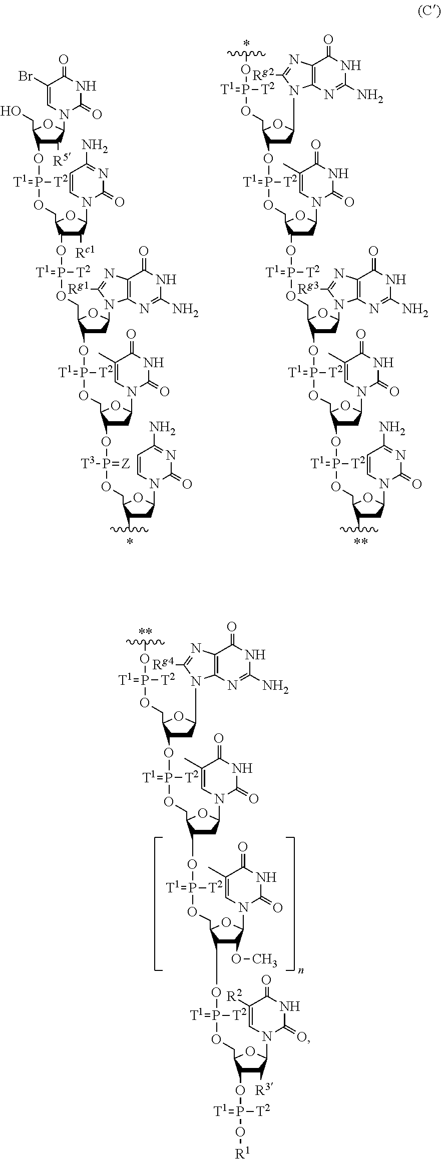

wherein:
[symbol] * and [symbol] ** indicate the points of attachment within the oligonucleotide;
each T¹ is independently O or S;
each T² is S⁻;
T³ is a group

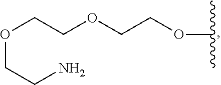 [SMILES: *OCCOCCOCCN]

wherein [symbol] indicates the point of attachment to the rest of the oligonucleotide;
Z is O or S;
R^{5′} is —H or methoxy;
R^{c1} is —H or methoxy;
R^{g1}, R^{g2}, R^{g3}, and R^{g4} are H;
R^{3′} is methoxy;
R¹ is —(CH₂)₃—OH;
R² is —H or methyl; and
n is an integer from 0 to 2,
   or a pharmaceutically acceptable salt thereof.

In some embodiments, the immunomodulatory oligonucleotide comprises the structure of formula (C″),

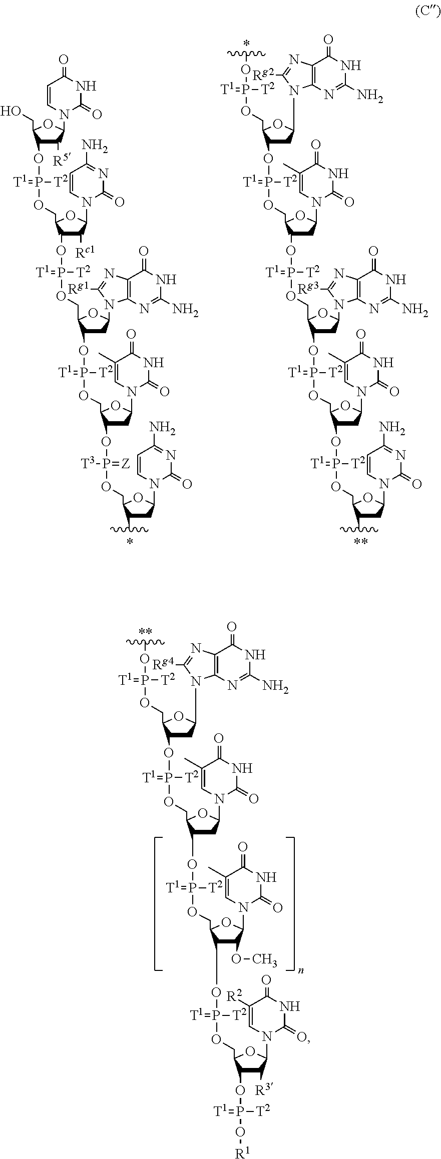

wherein:
[symbol] * and [symbol] ** indicate the points of attachment within the oligonucleotide;
each T¹ is independently O or S;
each T² is S⁻;
T³ is a group

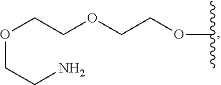 [SMILES: *OCCOCCOCCN]

wherein [symbol] indicates the point of attachment to the rest of the oligonucleotide;
Z is O or S;
R^{5′} is —H or methoxy;
R^{c1} is —H or methoxy;
R^{g1}, R^{g2}, R^{g3}, and R^{g4} are H;
R^{3′} is methoxy;
R¹ is —(CH₂)₃—OH;
R² is —H or methyl; and
n is an integer from 0 to 2,
   or a pharmaceutically acceptable salt thereof.

In some embodiments of the present aspect, Z is S. In additional embodiments, the oligonucleotide comprises at least one pair of geminal T¹ and T² wherein T¹ is S and T² is S⁻. In certain embodiments, the oligonucleotide comprises at least two pairs of geminal T¹ and T² wherein T¹ is S and T² is S⁻. The pair(s) of geminal T¹ and T² wherein T¹ is S and T² is S⁻may also be described as phosphorodithioate linkages.

It should be recognized that in some instances wherein the oligonucleotide has at least one pair of geminal T¹ and T² wherein T¹ is S and T² is S⁻, the phosphorodithioate linkage(s) may be further described in terms of the position within the oligonucleotide at which the linkage is located. The position of the linkage may be characterized, for example, as being between two nucleoside residues, e.g., between the first and second nucleoside residues (or between nucleoside residues 1 and 2) as counted from the 5′ end of the oligonucleotide. Alternatively, the position of the linkage may be described as being located at the 3′-position of a given nucleoside residue, e.g., on the internucleoside linker immediately following the specified nucleoside residue or the 3′-position of the′3-terminal residue.

In some embodiments wherein the oligonucleotide comprises at least one pair of geminal T¹ and T² wherein T¹ is S and T² is S⁻, and wherein n is 0, the at least one phosphorodithioate linkage is between nucleoside residues 1 and 2, between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, between nucleoside residues 10 and 11, or between nucleoside residues 11 and 12. In some embodiments wherein the oligonucleotide comprises at least one pair of geminal T¹ and T² wherein T¹ is S and T² is S⁻, and wherein n is 0, the at least one phosphorodithioate linkage is located at the 3′-position of nucleoside residue 1, nucleoside residue 2, nucleoside residue 3, nucleoside residue 5, nucleoside residue 6, nucleoside residue 7, nucleoside residue 8, nucleoside residue 9, nucleoside residue 10, nucleoside residue 11, nucleoside residue 12, or nucleoside residue 13.

In some embodiments wherein the oligonucleotide comprises at least one pair of geminal T¹ and T² wherein T¹ is S and T² is S⁻, and wherein n is 1, the at least one phosphorodithioate linkage is between nucleoside residues 1 and 2, between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, between nucleoside residues 10 and 11, between nucleoside residues 11 and 12, or between nucleoside residues 12 and 13. In some embodiments wherein the oligonucleotide comprises at least one pair of geminal T¹ and T² wherein T¹ is S and T² is S⁻, and wherein n is 0, the at least one phosphorodithioate linkage is located at the 3′-position of nucleoside residue 1, nucleoside residue 2, nucleoside residue 3, nucleoside residue 5, nucleoside residue 6, nucleoside residue 7, nucleoside residue 8, nucleoside residue 9, nucleoside residue 10, nucleoside residue 11, nucleoside residue 12, nucleoside residue 13, or nucleoside residue 14.

In some embodiments wherein the oligonucleotide comprises at least one pair of geminal T¹ and T² wherein T¹ is S and T² is S⁻, and wherein n is 1, the at least one phosphorodithioate linkage is between nucleoside residues 1 and 2, between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, between nucleoside residues 10 and 11, between nucleoside residues 11 and 12, or between nucleoside residues 12 and 13. In some embodiments wherein the oligonucleotide comprises at least one pair of geminal T¹ and T² wherein T¹ is S and T² is S⁻, and wherein n is 1, the at least one phosphorodithioate linkage is located at the 3′-position of nucleoside residue 1, nucleoside residue 2, nucleoside residue 3, nucleoside residue 5, nucleoside residue 6, nucleoside residue 7, nucleoside residue 8, nucleoside residue 9, nucleoside residue 10, nucleoside residue 11, nucleoside residue 12, nucleoside residue 13, or nucleoside residue 14.

In some embodiments wherein the oligonucleotide comprises at least one pair of geminal T¹ and T² wherein T¹ is S and T² is S⁻, and wherein n is 2, the at least one phosphorodithioate linkage is between nucleoside residues 1 and 2, between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, between nucleoside residues 10 and 11, between nucleoside residues 11 and 12, between nucleoside residues 12 and 13, or between residues 13 and 14. In some embodiments wherein the oligonucleotide comprises at least one pair of geminal T¹ and T² wherein T¹ is S and T² is S⁻, and wherein n is 2, the at least one phosphorodithioate linkage is located at the 3′-position of nucleoside residue 1, nucleoside residue 2, nucleoside residue 3, nucleoside residue 5, nucleoside residue 6, nucleoside residue 7, nucleoside residue 8, nucleoside residue 9, nucleoside residue 10, nucleoside residue 11, nucleoside residue 12, nucleoside residue 13, nucleoside residue 14, or residue 15.

In still other embodiments wherein the oligonucleotide has at least two phosphorodithioate linkages or comprises at least two pairs of geminal T¹ and T² wherein T¹ is S and T² is S⁻, the positions of one or both phosphorodithioate linkages or pairs of T¹ and T² may be specified. It should be recognized that the positions of one or both phosphorodithioate linkages may be independently varied.

In some embodiments the immunomodulatory oligonucleotide comprises the structure of formula (C′),

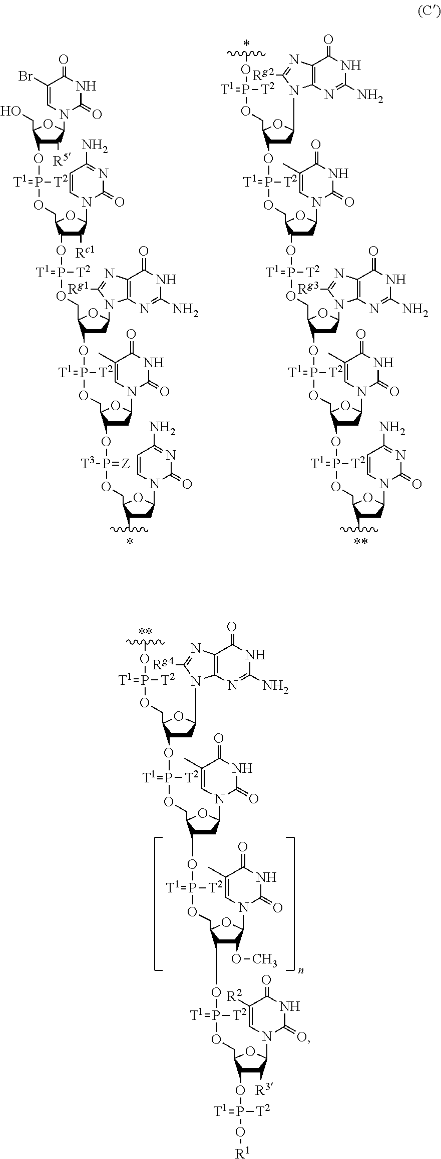

wherein:
[symbol] * and [symbol] **indicate the points of attachment within the oligonucleotide;
each T¹ is independently O or S;
each T² is S⁻;
T³ is a group

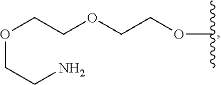 [SMILES: *OCCOCCOCCN]

wherein [symbol] indicates the point of attachment to the rest of the oligonucleotide;
Z is O or S;
R^{5′} is —H or methoxy;
R^{c1} is —H or methoxy;
R^{g1}, R^{g2}, R^{g3}, and R^{g4} are H;
R^{3′} is methoxy;
R¹ is —(CH₂)₃—OH;
R² is —H or methyl; and
n is an integer from 0 to 2,
   or a pharmaceutically acceptable salt thereof.

In some embodiments the immunomodulatory oligonucleotide comprises the structure of formula (C″),

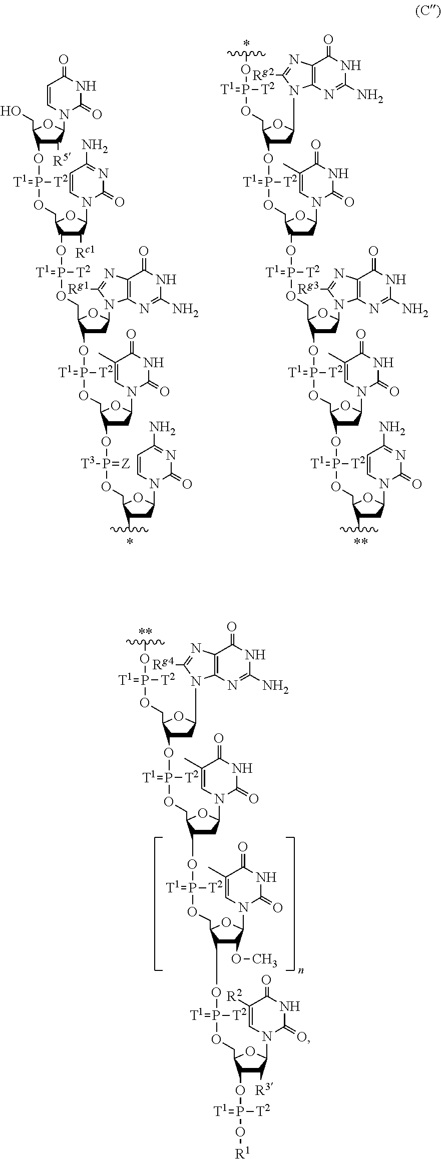

wherein:
[symbol] * and [symbol] ** indicate the points of attachment within the oligonucleotide;
each T¹ is independently O or S;
each T² is S⁻;
T³ is a group

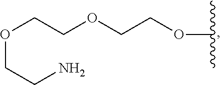 [SMILES: *OCCOCCOCCN]

wherein [symbol] indicates the point of attachment to the rest of the oligonucleotide;
Z is O or S;
R^{5′} is —H or methoxy;
R¹ is —H or methoxy;
R^{g1}, R^{g2}, R^{g3}, and R^{g4} are H;
R^{3′} is methoxy;
R¹ is —(CH₂)₃—OH;
R² is —H or methyl; and
n is an integer from 0 to 2,
   or a pharmaceutically acceptable salt thereof.

In some embodiments of the present aspect, Z is S. In additional embodiments, the oligonucleotide comprises at least one pair of geminal T¹ and T² wherein T¹ is S and T² is S⁻. In certain embodiments, the oligonucleotide comprises at least two pairs of geminal T¹ and T² wherein T¹ is S and T² is S⁻.

In some embodiments the oligonucleotide comprises the structure of formula (C),

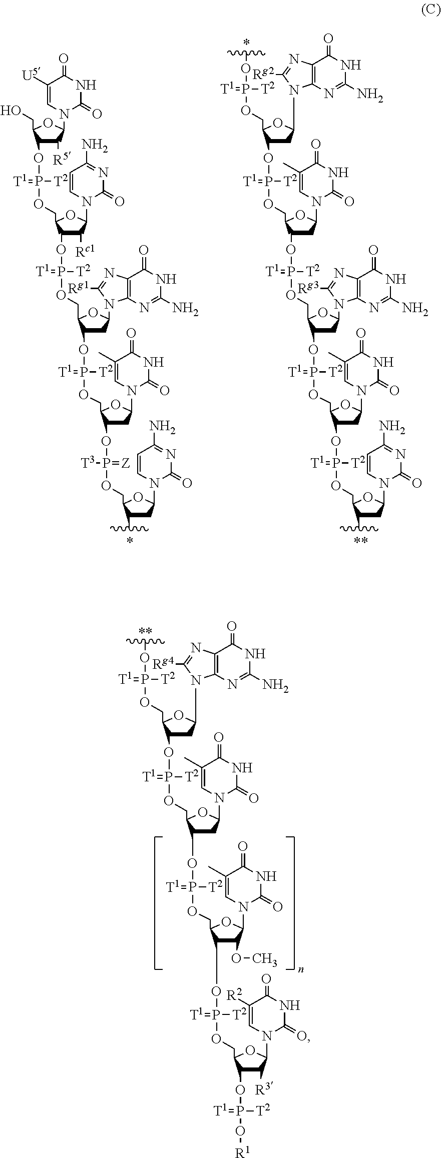

wherein [symbol] * and [symbol] ** indicate the points of attachment within the oligonucleotide;
each T¹ is independently O or S;
each T² is S⁻;
provided that the oligonucleotide comprises at least one pair of geminal T¹ and T² wherein T¹ is S and T² is S,
T³ is a group

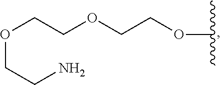 [SMILES: *OCCOCCOCCN]

wherein [symbol] indicates the point of attachment to the rest of the oligonucleotide;
Z is O or S;
U^{5′} is —H or halogen;
R^{5′} is —H;
R^{c1} is —H;
R^{g1}, R^{g2}, R^{g3}, and R^{g4} are H;
R^{3′} is methoxy;
R¹ is —(CH₂)₃—OH;
R² is -methyl; and
n is 1,
or a pharmaceutically acceptable salt thereof.

In some embodiments of any of the foregoing, the at least one pair of geminal T¹ and T² wherein T¹ is S and T² is S is between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11. In still other embodiments of the foregoing, the oligonucleotide comprises at least two pairs of of geminal T¹ and T² wherein T¹ is S and T² is S, and wherein the at least two pairs of of geminal T¹ and T² wherein T¹ is S and T² is S are between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11.

In some embodiments, the oligonucleotide comprises one or two pairs of geminal T¹ and T² wherein T¹ is S and T² is S, and wherein the one or two pairs of geminal T¹ and T² are between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11. In certain embodiments, the oligonucleotide comprises one pair of geminal T¹ and T² wherein T¹ is S and T² is S, and wherein the pair of geminal T¹ and T² is between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11. In certain other embodiments, the oligonucleotide comprises two pairs of geminal T¹ and T² wherein T¹ is S and T² is S, and wherein the two pairs of geminal T¹ and T² wherein T¹ is S and T² is S are between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11

In some embodiments, R^{5′} is H. In other embodiments, R^{5′} is methoxy. In some embodiments, R^{c1} is H. In yet other embodiments, R^{c1} is methoxy. In still further embodiments, R² is methyl. In still other embodiments, R² is H. In yet other additional embodiments, which may be combined with any of the preceding embodiments, T³ is

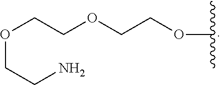 [SMILES: *OCCOCCOCCN]

In still other embodiments, T³ is

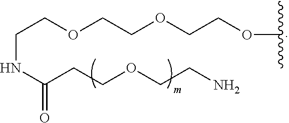

In certain embodiments, m is an integer from 20 to 25.

In another aspect, the immunomodulating oligonucleotide of formula (C) is an oligonucleotide selected from the group consisting of the oligonucleotides of Table 3 and Table 4, or a pharmaceutically acceptable salt thereof. In still other embodiments, the immunomodulating oligonucleotide of formula (C) is an oligonucleotide selected from the group consisting of the oligonucleotides of Table 4, or a pharmaceutically acceptable salt thereof.

TABLE 3
Modified Oligonucleotide Structures (with PEG₃NH₂)

| Cmpd | |
|---|---|
| # | Structure |
| 1.1a | 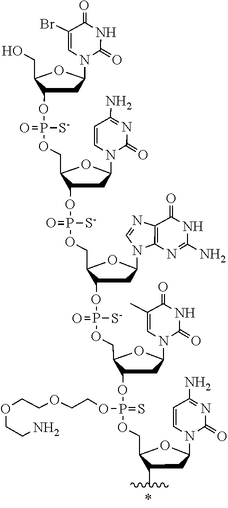 [SMILES: *[C@@H]1C[C@H](n2ccc(N)nc2=O)O[C@@H]1COP(=S)(OCCOCCOCCN)O[C@@H]1C[C@H](n2cc(C)c(=O)[nH]c2=O)O[C@@H]1COP(=O)([S-])O[C@@H]1C[C@H](n2cnc3c(=O)[nH]c(N)nc32)O[C@@H]1COP(=O)([S-])O[C@@H]1C[C@H](n2ccc(N)nc2=O)O[C@@H]1COP(=O)([S-])O[C@@H]1C[C@H](n2cc(Br)c(=O)[nH]c2=O)O[C@@H]1CO] |
| | 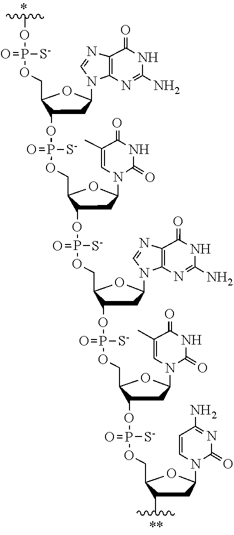 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 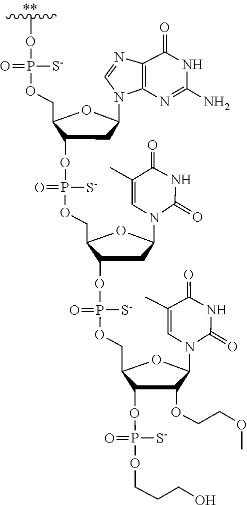 |
| 2.1a | 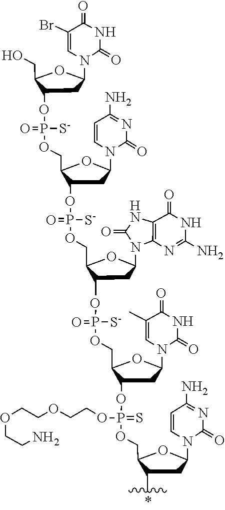 [SMILES: *[C@@H]1C[C@H](n2ccc(N)nc2=O)O[C@@H]1COP(=S)(OCCOCCOCCN)O[C@@H]1C[C@H](n2cc(C)c(=O)[nH]c2=O)O[C@@H]1COP(=O)([S-])O[C@@H]1C[C@H](n2c(=O)[nH]c3c(=O)[nH]c(N)nc32)O[C@@H]1COP(=O)([S-])O[C@@H]1C[C@H](n2ccc(N)nc2=O)O[C@@H]1COP(=O)([S-])O[C@@H]1C[C@H](n2cc(Br)c(=O)[nH]c2=O)O[C@@H]1CO] |
| | 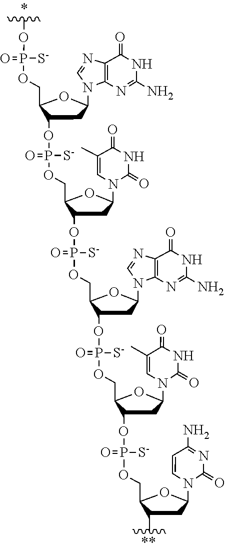 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 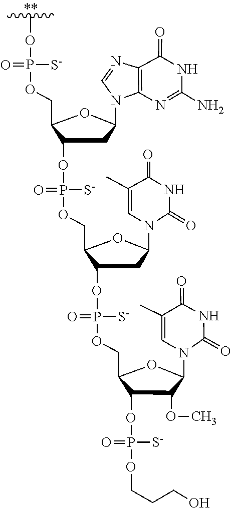 |
| 2.2a | 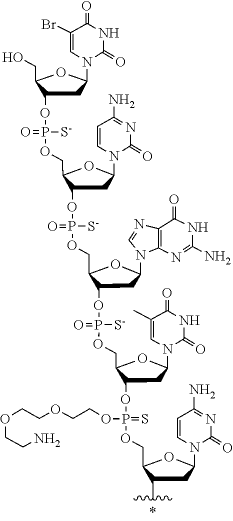 [SMILES: *[C@@H]1C[C@H](n2ccc(N)nc2=O)O[C@@H]1COP(=S)(OCCOCCOCCN)O[C@@H]1C[C@H](n2cc(C)c(=O)[nH]c2=O)O[C@@H]1COP(=O)([S-])O[C@@H]1C[C@H](n2cnc3c(=O)[nH]c(N)nc32)O[C@@H]1COP(=O)([S-])O[C@@H]1C[C@H](n2ccc(N)nc2=O)O[C@@H]1COP(=O)([S-])O[C@@H]1C[C@H](n2cc(Br)c(=O)[nH]c2=O)O[C@@H]1CO] |
| | 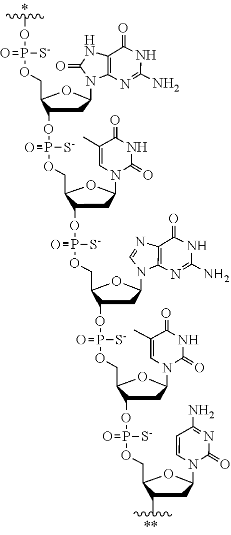 [SMILES: *OP(=O)([S-])OC[C@H]1O[C@@H](n2c(=O)[nH]c3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 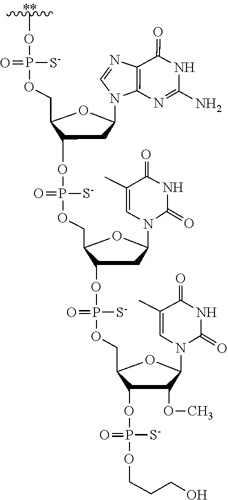 |
| 2.3a | 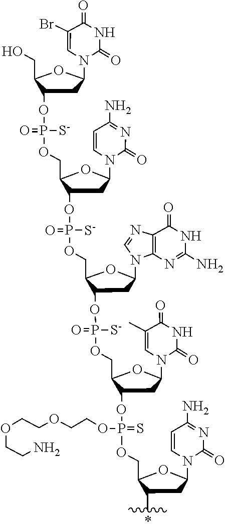 [SMILES: *[C@@H]1C[C@H](n2ccc(N)nc2=O)O[C@@H]1COP(=S)(OCCOCCOCCN)O[C@@H]1C[C@H](n2cc(C)c(=O)[nH]c2=O)O[C@@H]1COP(=O)([S-])O[C@@H]1C[C@H](n2cnc3c(=O)[nH]c(N)nc32)O[C@@H]1COP(=O)([S-])O[C@@H]1C[C@H](n2ccc(N)nc2=O)O[C@@H]1COP(=O)([S-])O[C@@H]1C[C@H](n2cc(Br)c(=O)[nH]c2=O)O[C@@H]1CO] |
| | 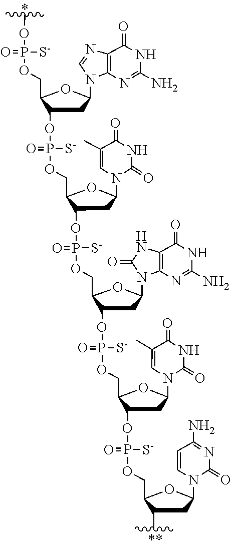 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2c(=O)[nH]c3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 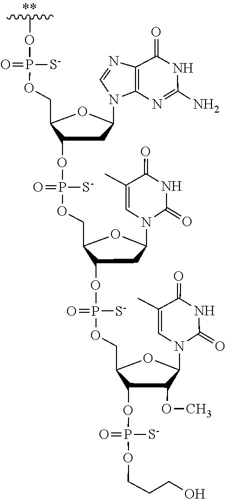 |
| 2.4a | 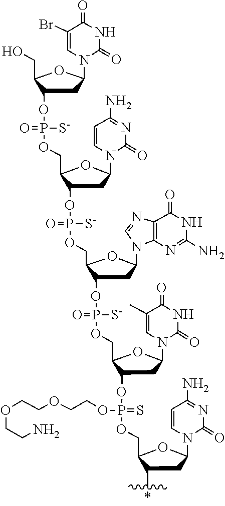 [SMILES: *[C@@H]1C[C@H](n2ccc(N)nc2=O)O[C@@H]1COP(=S)(OCCOCCOCCN)O[C@@H]1C[C@H](n2cc(C)c(=O)[nH]c2=O)O[C@@H]1COP(=O)([S-])O[C@@H]1C[C@H](n2cnc3c(=O)[nH]c(N)nc32)O[C@@H]1COP(=O)([S-])O[C@@H]1C[C@H](n2ccc(N)nc2=O)O[C@@H]1COP(=O)([S-])O[C@@H]1C[C@H](n2cc(Br)c(=O)[nH]c2=O)O[C@@H]1CO] |
| | 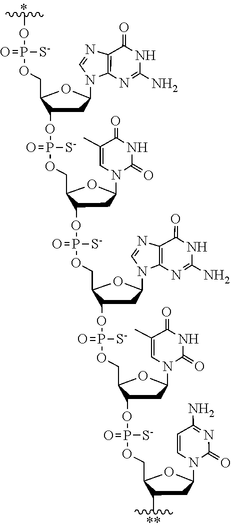 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 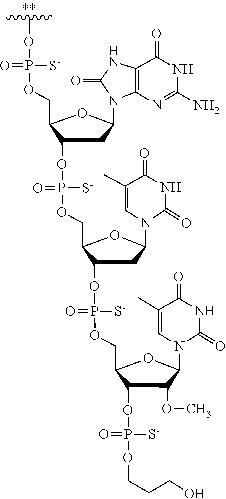 |

TABLE 4
Modified Oligonucleotide Structures (with —PEG₃NH₂)

| Cmpd | |
|---|---|
| # | Structure |
| 3.1a | 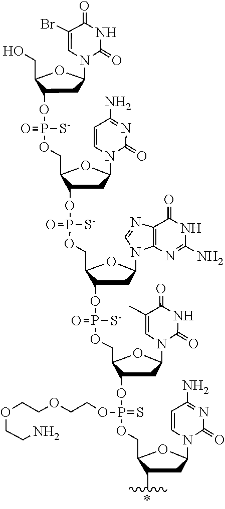 [SMILES: *[C@@H]1C[C@H](n2ccc(N)nc2=O)O[C@@H]1COP(=S)(OCCOCCOCCN)O[C@@H]1C[C@H](n2cc(C)c(=O)[nH]c2=O)O[C@@H]1COP(=O)([S-])O[C@@H]1C[C@H](n2cnc3c(=O)[nH]c(N)nc32)O[C@@H]1COP(=O)([S-])O[C@@H]1C[C@H](n2ccc(N)nc2=O)O[C@@H]1COP(=O)([S-])O[C@@H]1C[C@H](n2cc(Br)c(=O)[nH]c2=O)O[C@@H]1CO] |
| | 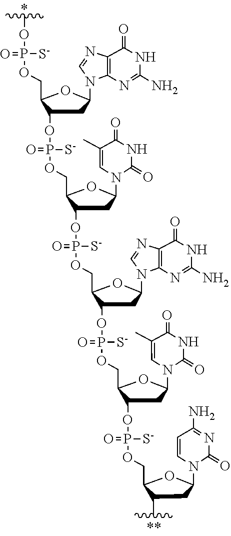 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 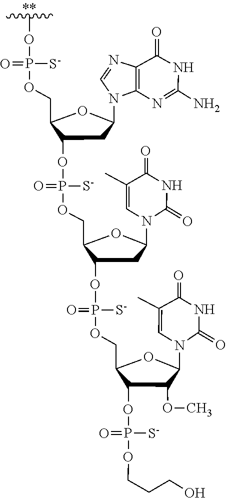 |
| 3.2a | 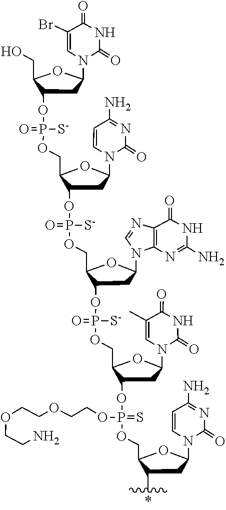 [SMILES: *[C@@H]1C[C@H](n2ccc(N)nc2=O)O[C@@H]1COP(=S)(OCCOCCOCCN)O[C@@H]1C[C@H](n2cc(C)c(=O)[nH]c2=O)O[C@@H]1COP(=O)([S-])O[C@@H]1C[C@H](n2cnc3c(=O)[nH]c(N)nc32)O[C@@H]1COP(=O)([S-])O[C@@H]1C[C@H](n2ccc(N)nc2=O)O[C@@H]1COP(=O)([S-])O[C@@H]1C[C@H](n2cc(Br)c(=O)[nH]c2=O)O[C@@H]1CO] |
| | 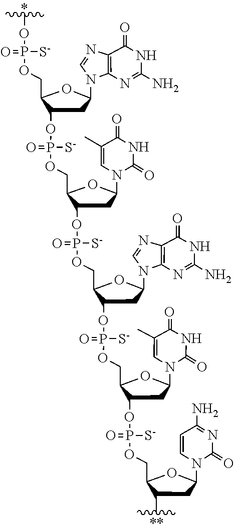 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 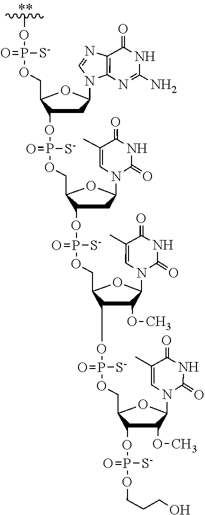 |
| 3.3a | 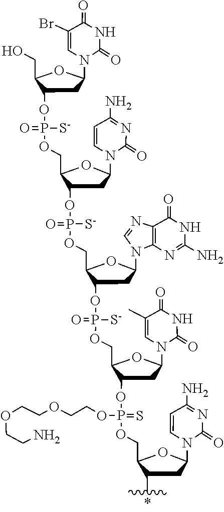 [SMILES: *[C@@H]1C[C@H](n2ccc(N)nc2=O)O[C@@H]1COP(=S)(OCCOCCOCCN)O[C@@H]1C[C@H](n2cc(C)c(=O)[nH]c2=O)O[C@@H]1COP(=O)([S-])O[C@@H]1C[C@H](n2cnc3c(=O)[nH]c(N)nc32)O[C@@H]1COP(=O)([S-])O[C@@H]1C[C@H](n2ccc(N)nc2=O)O[C@@H]1COP(=O)([S-])O[C@@H]1C[C@H](n2cc(Br)c(=O)[nH]c2=O)O[C@@H]1CO] |
| | 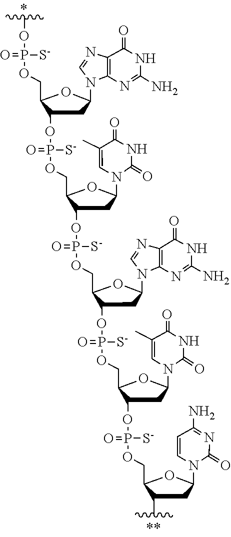 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 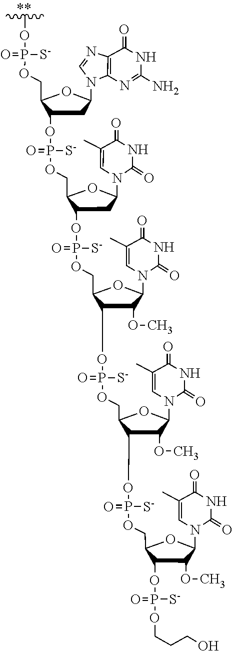 |
| 4.1a | 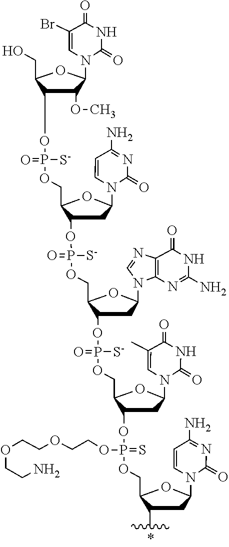 |
| | 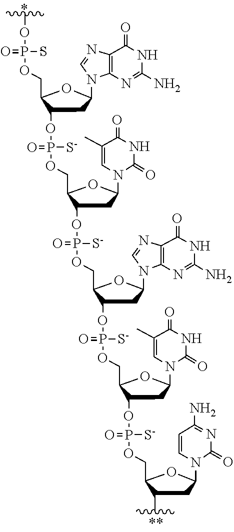 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 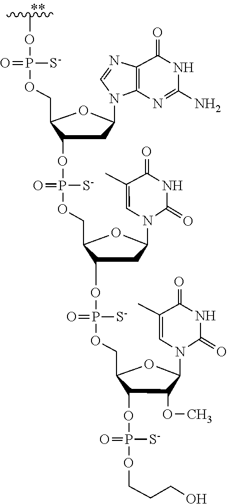 |
| 4.2a | 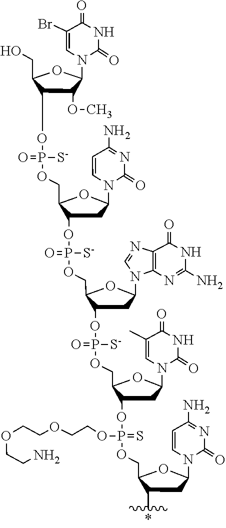 |
| | 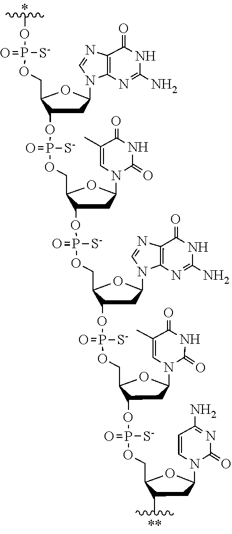 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 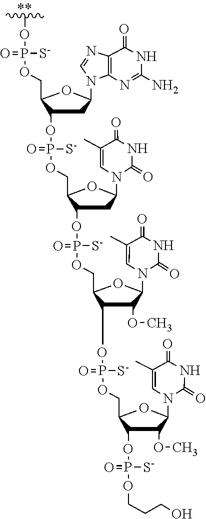 |
| 4.3a | 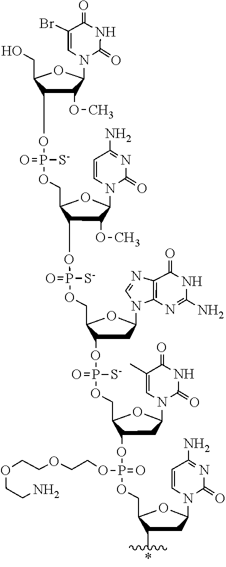 |
| | 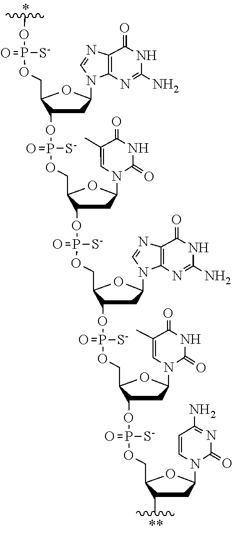 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 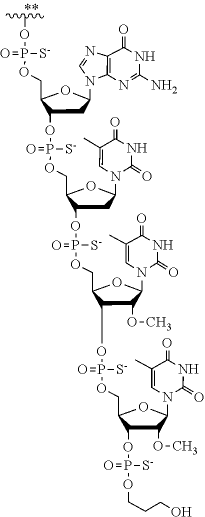 |
| 5.1a | 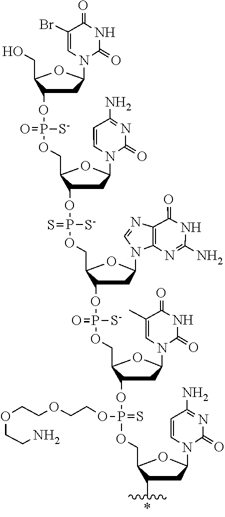 [SMILES: *[C@@H]1C[C@H](n2ccc(N)nc2=O)O[C@@H]1COP(=S)(OCCOCCOCCN)O[C@@H]1C[C@H](n2cc(C)c(=O)[nH]c2=O)O[C@@H]1COP(=O)([S-])O[C@@H]1C[C@H](n2cnc3c(=O)[nH]c(N)nc32)O[C@@H]1COP(=S)([S-])O[C@@H]1C[C@H](n2ccc(N)nc2=O)O[C@@H]1COP(=O)([S-])O[C@@H]1C[C@H](n2cc(Br)c(=O)[nH]c2=O)O[C@@H]1CO] |
| | 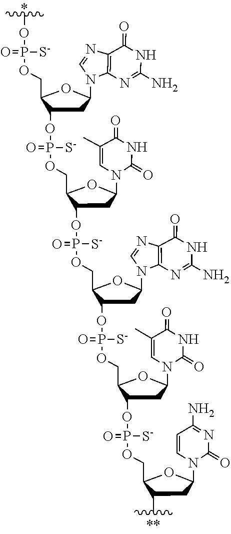 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 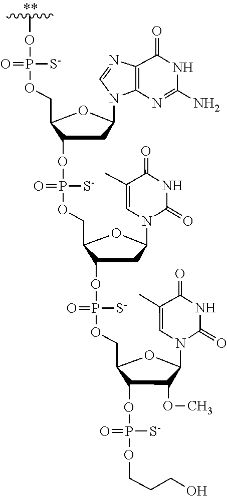 |
| 5.2a | 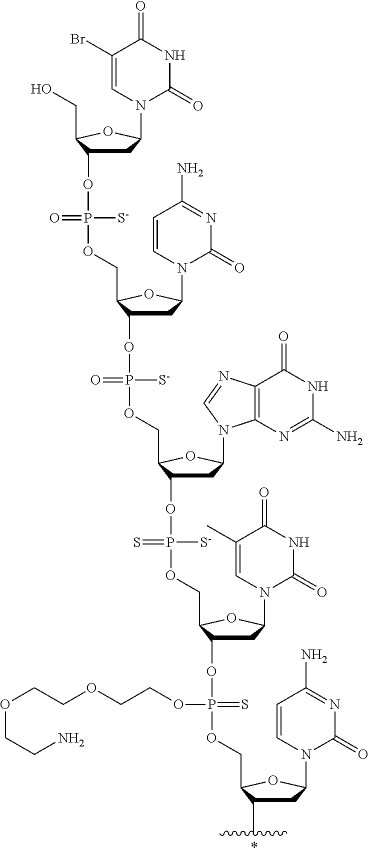 |
| | 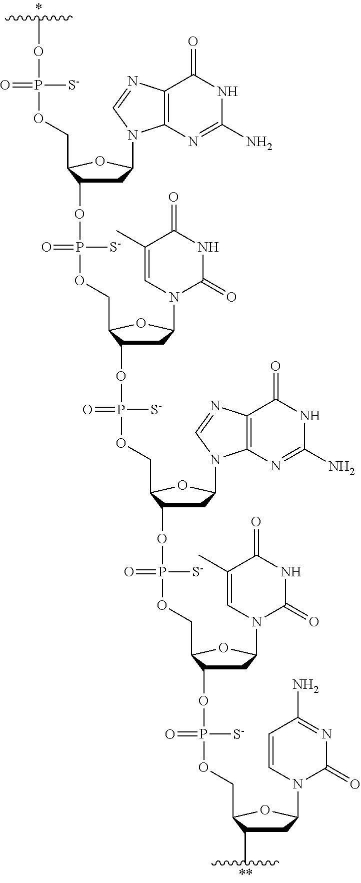 |
| | 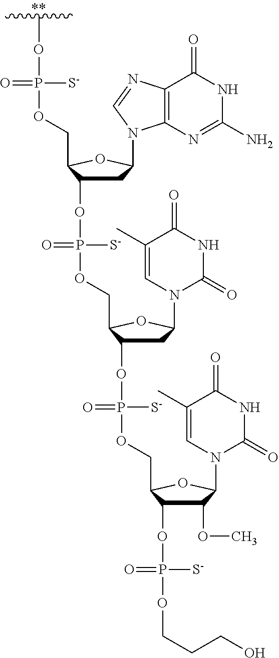 |
| 5.3a | 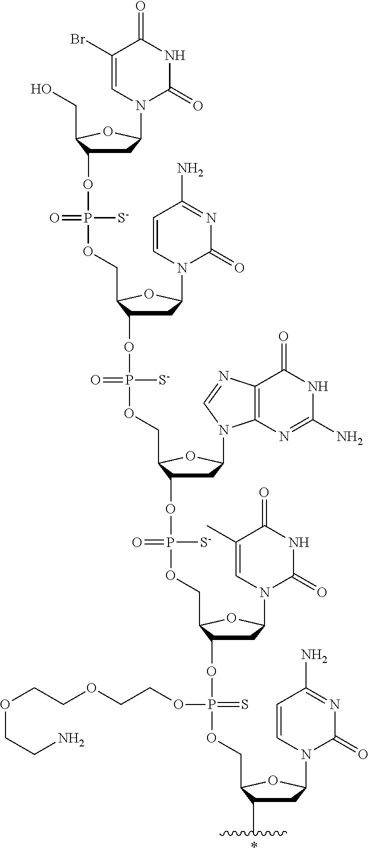 |
| | 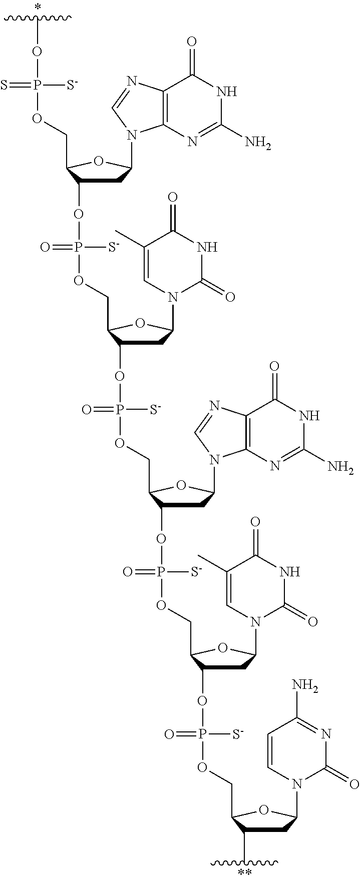 |
| | 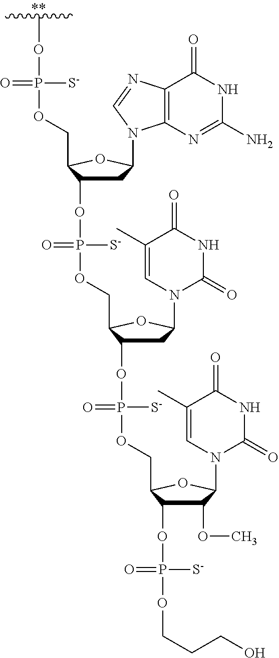 |
| 5.4a | 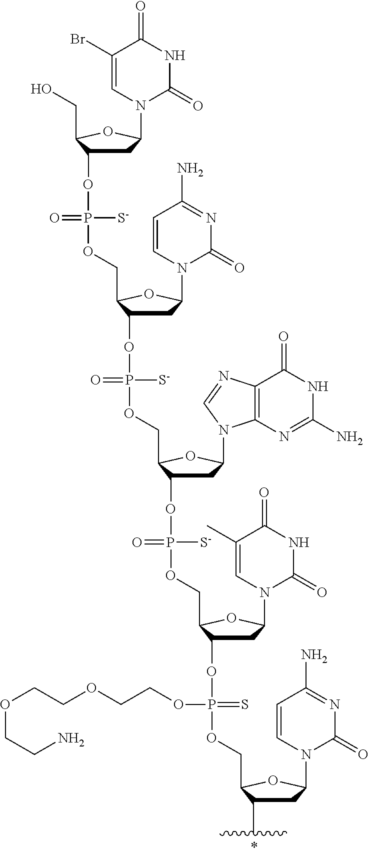 |
| | 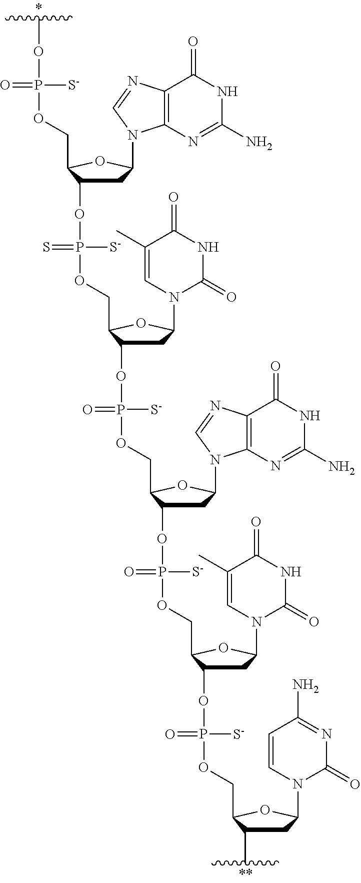 |
| | 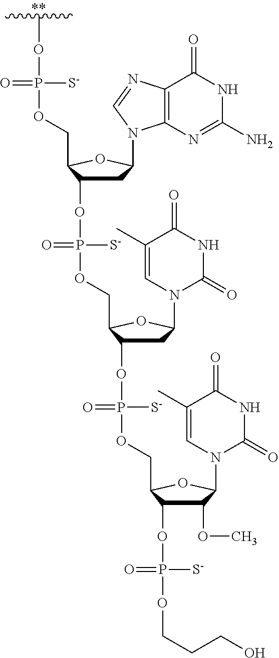 |
| 5.5a | 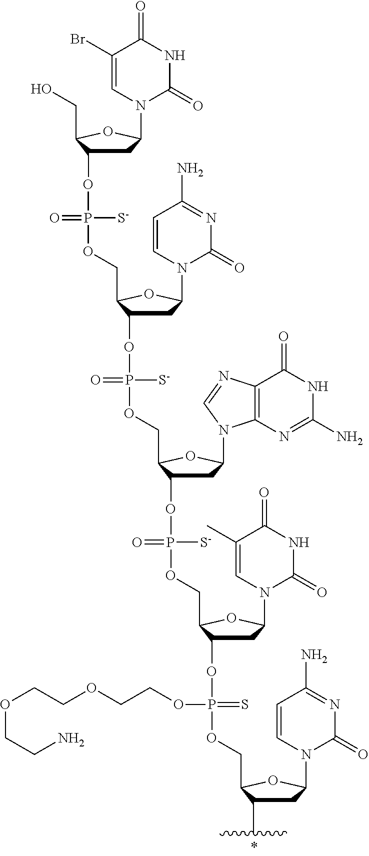 |
| | 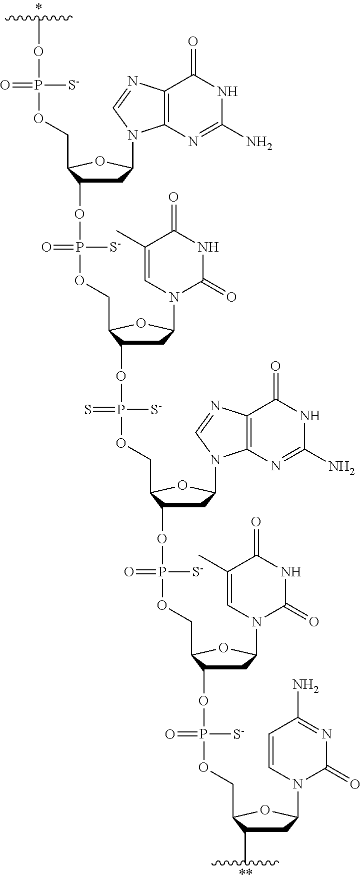 |
| | 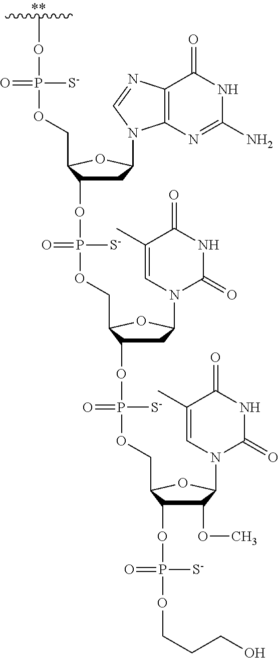 |
| 5.6a | 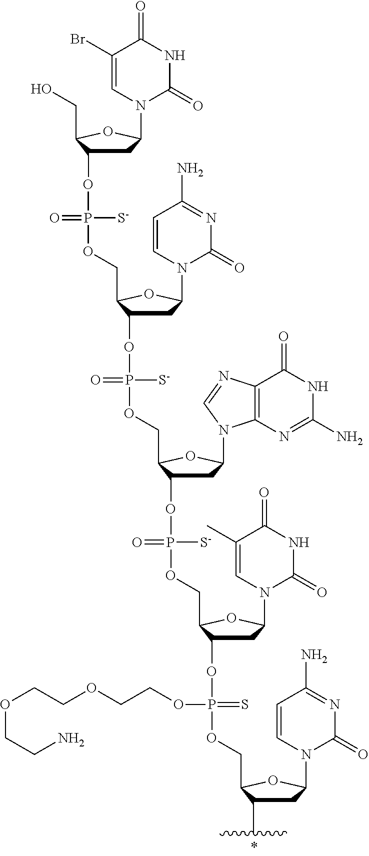 |
| | 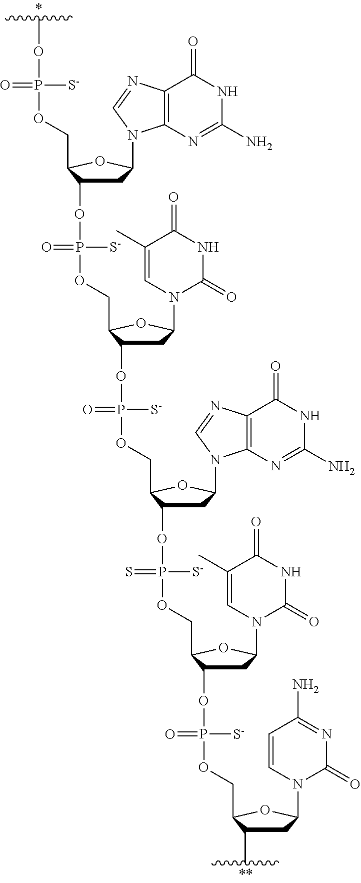 |
| | 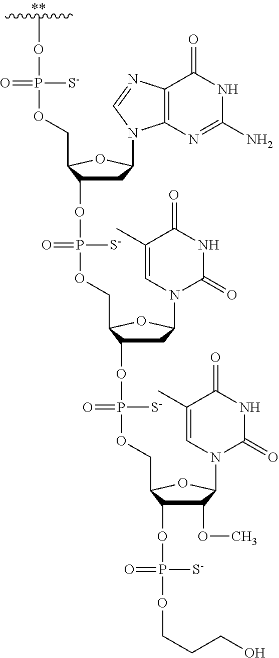 |
| 5.7a | 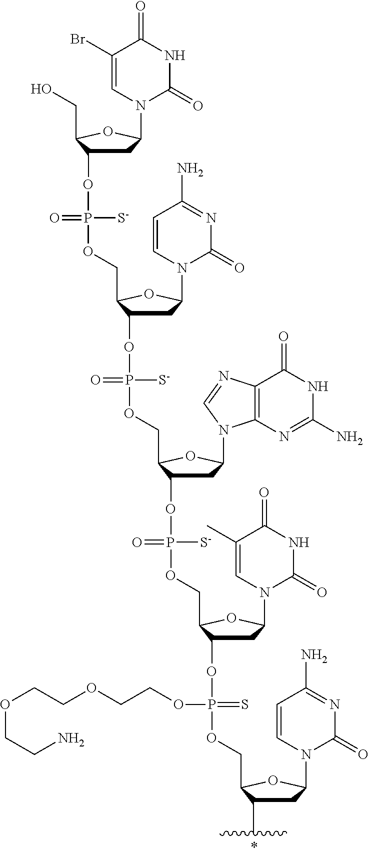 |
| | 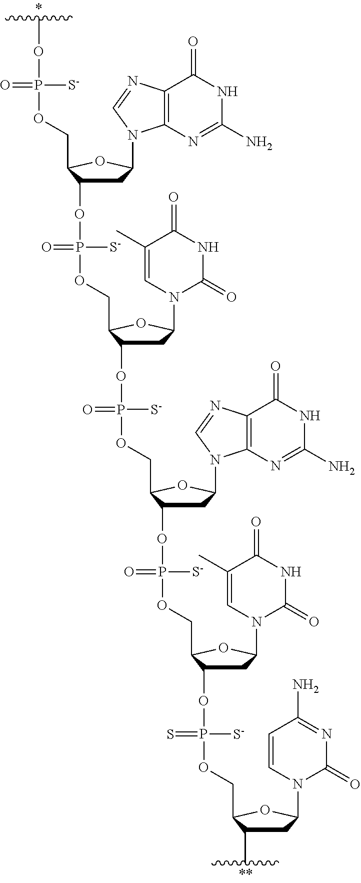 |
| | 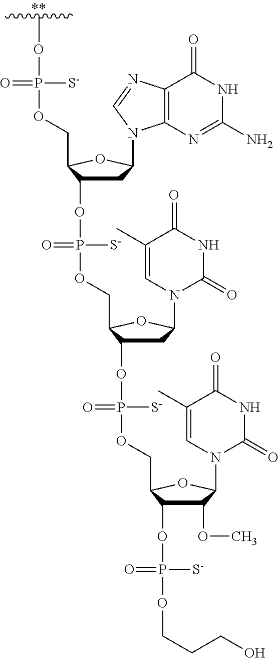 |
| 5.8a | 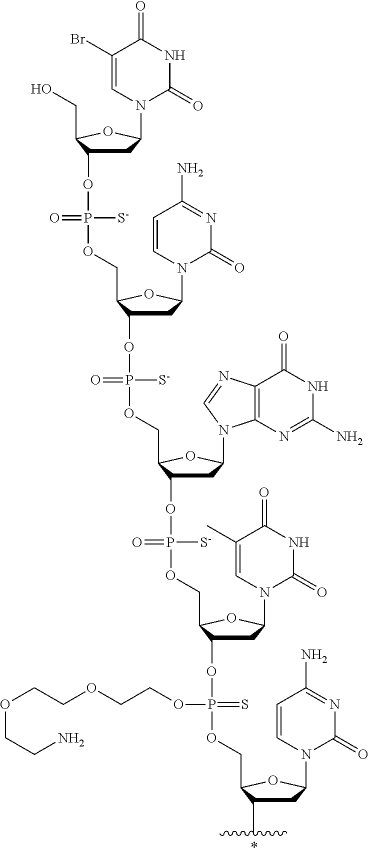 |
| | 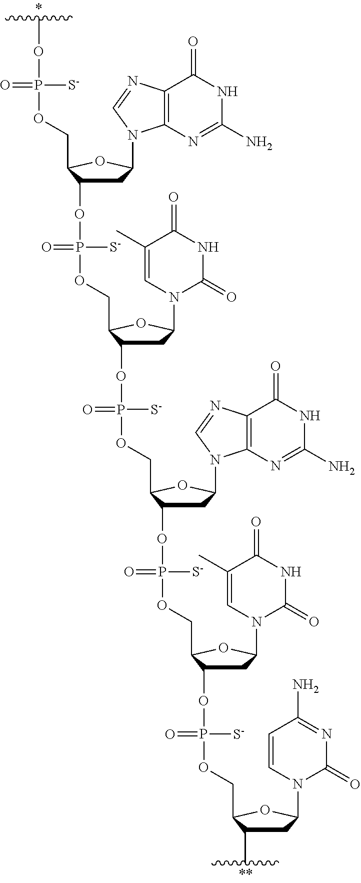 |
| | 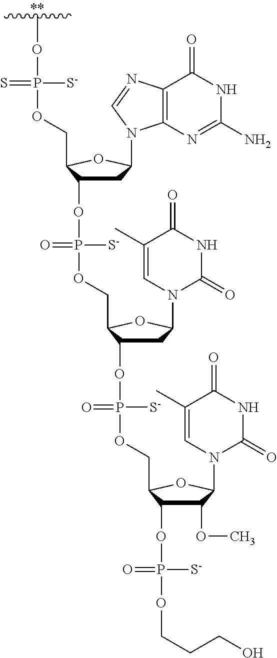 |
| 5.9a | 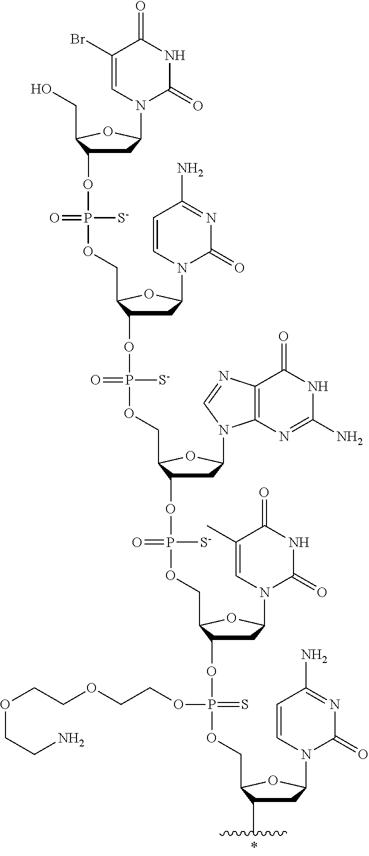 |
| | 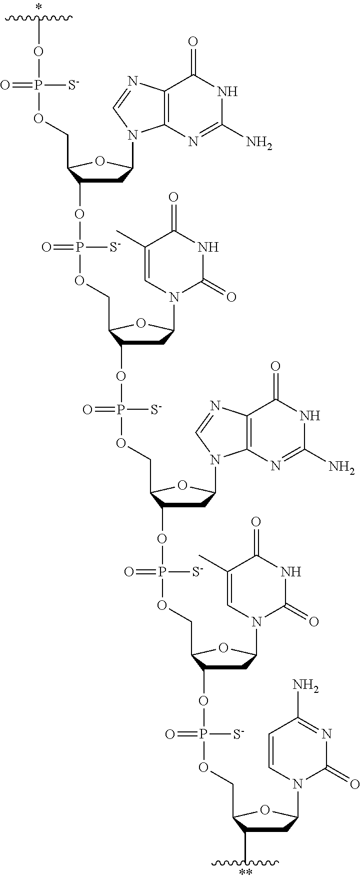 |
| | 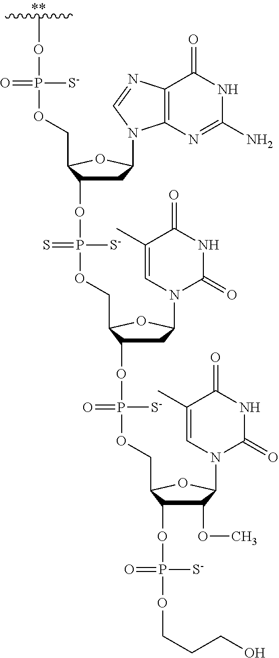 |
| 5.10a | 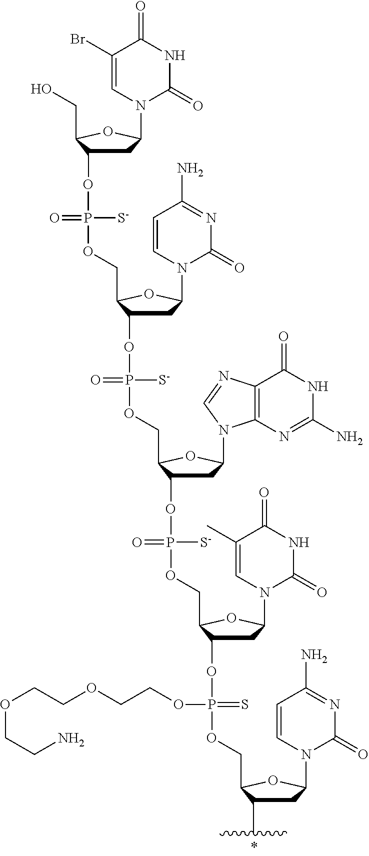 |
| | 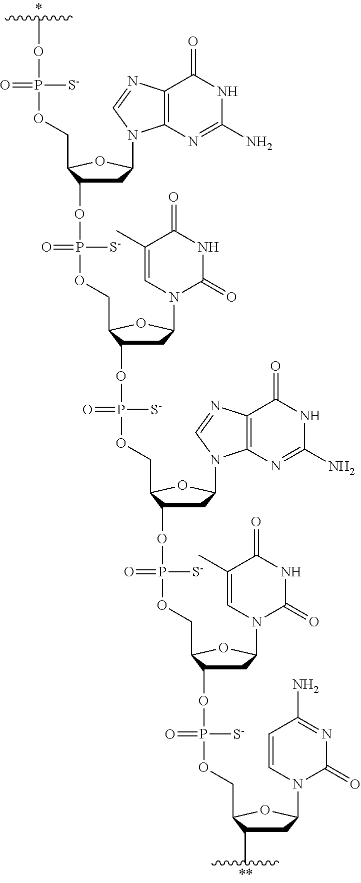 |
| | 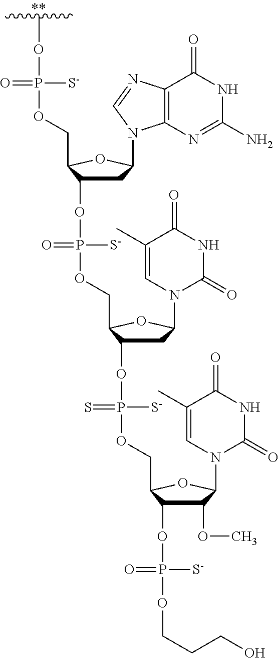 |
| 5.11a | 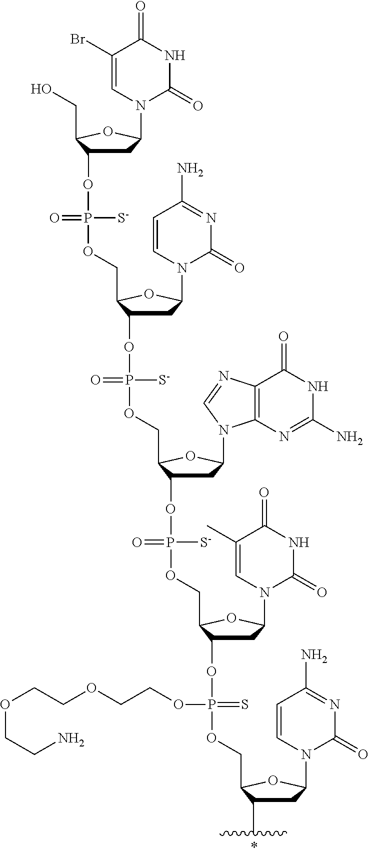 |
| | 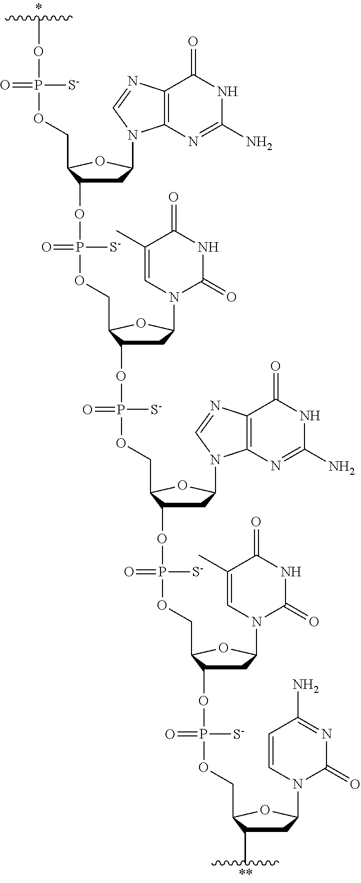 |
| | 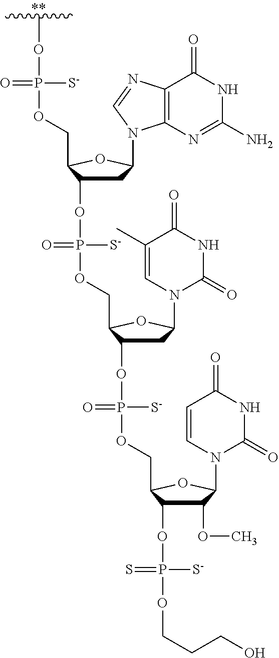 |
| 5.12a | 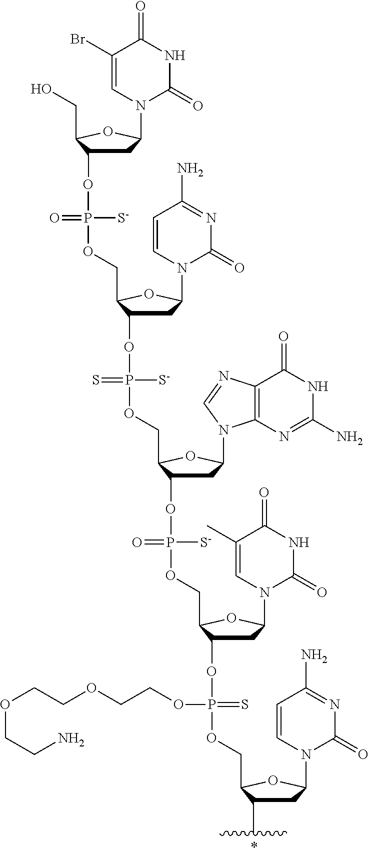 |
| | 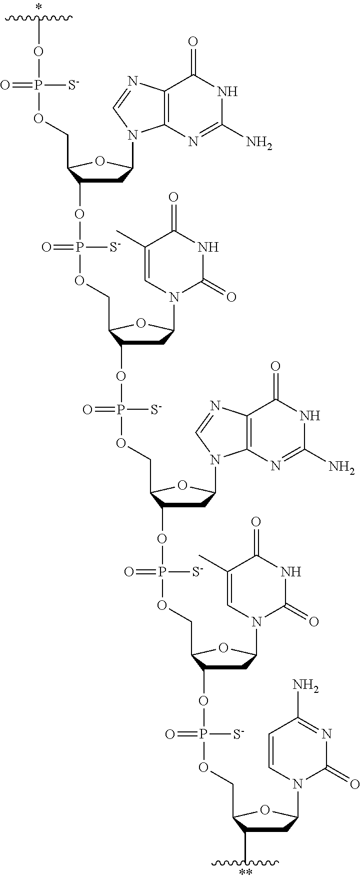 |
| | 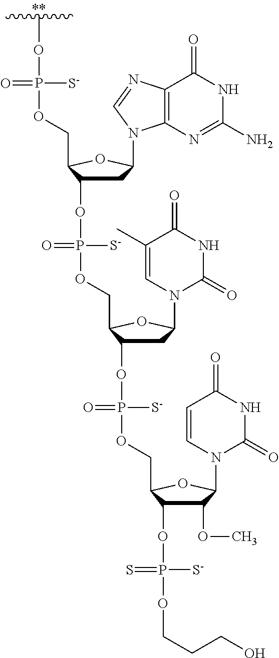 |
| 6.1a | 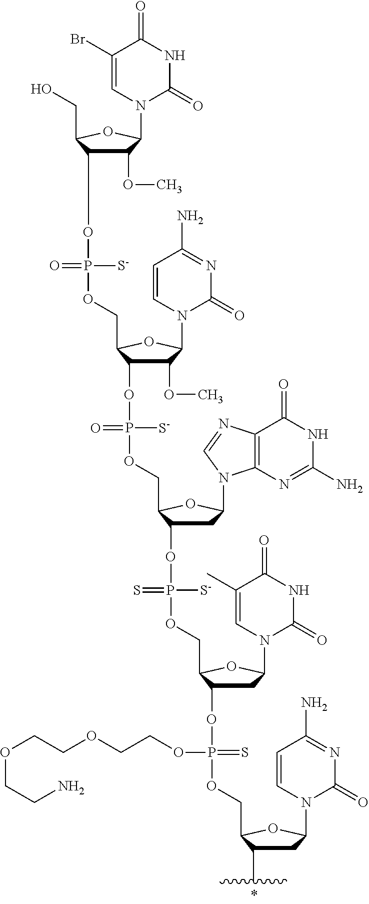 |
| | 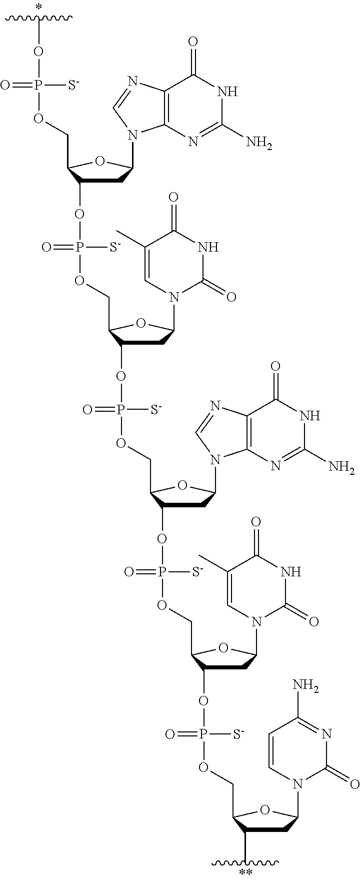 |
| | 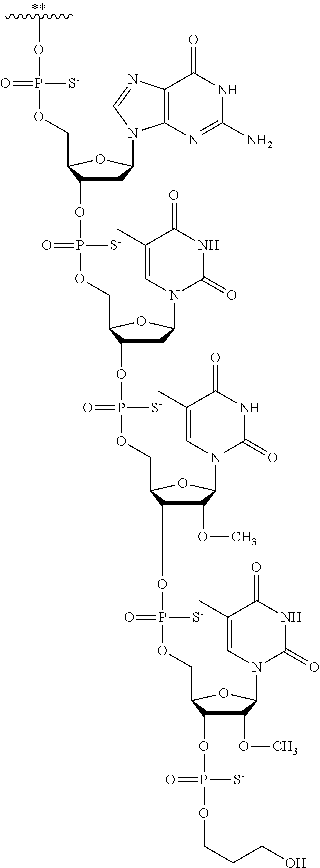 |
| 6.2a | 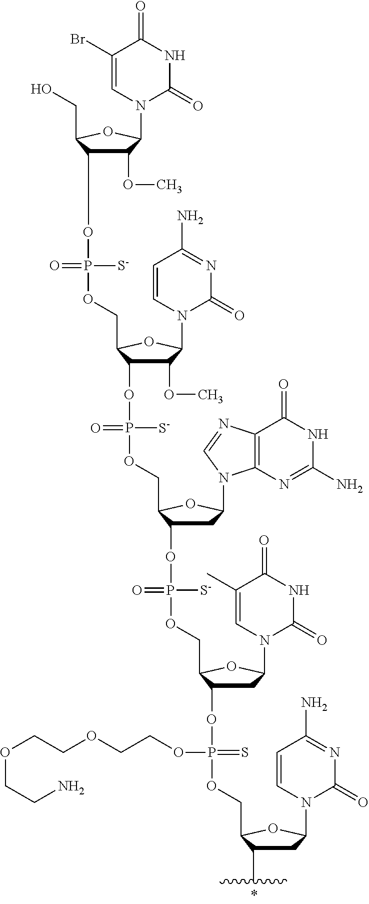 |
| | 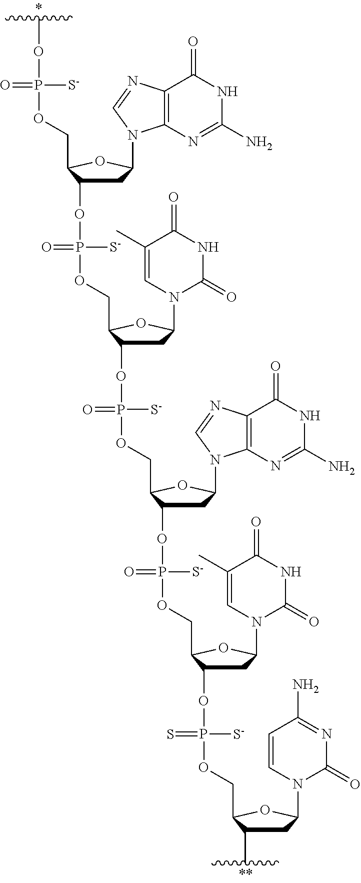 |
| | 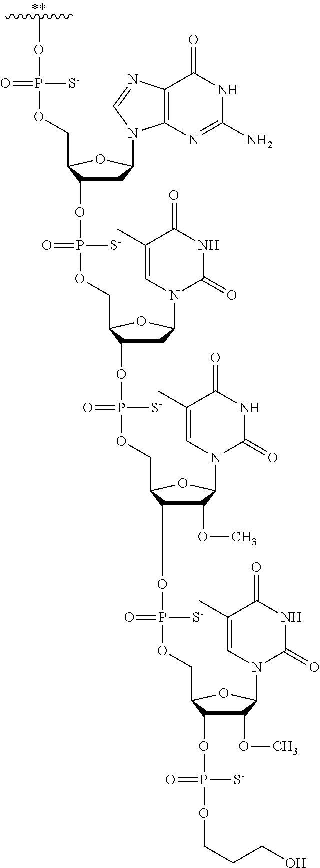 |
| 6.3a | 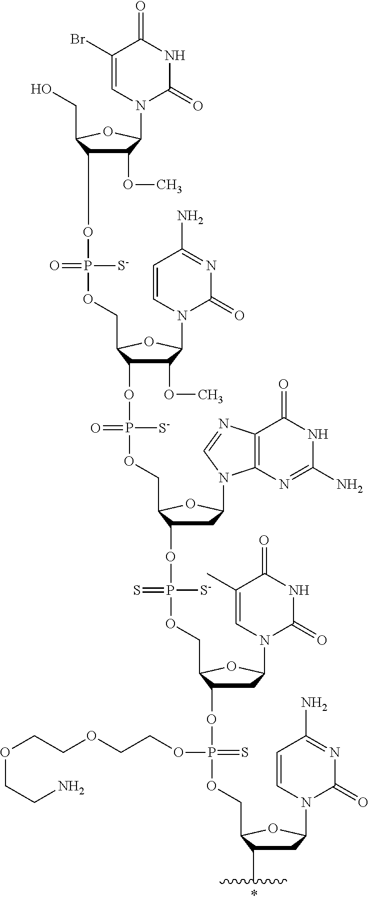 |
| | 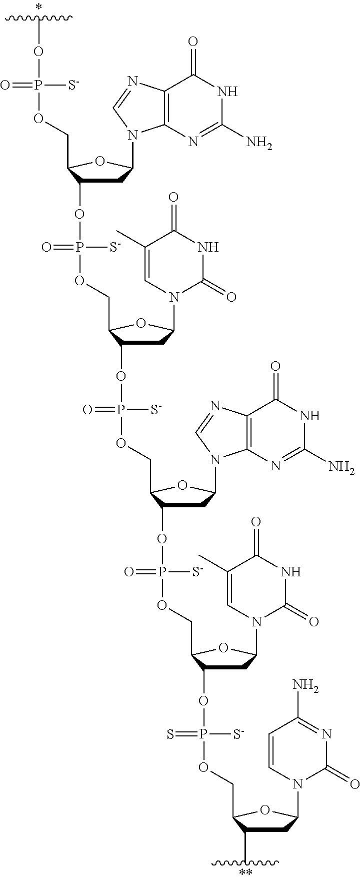 |
| | 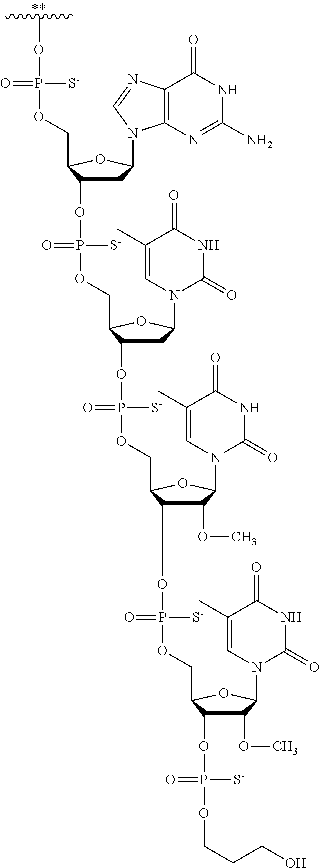 |
| 7.1a | 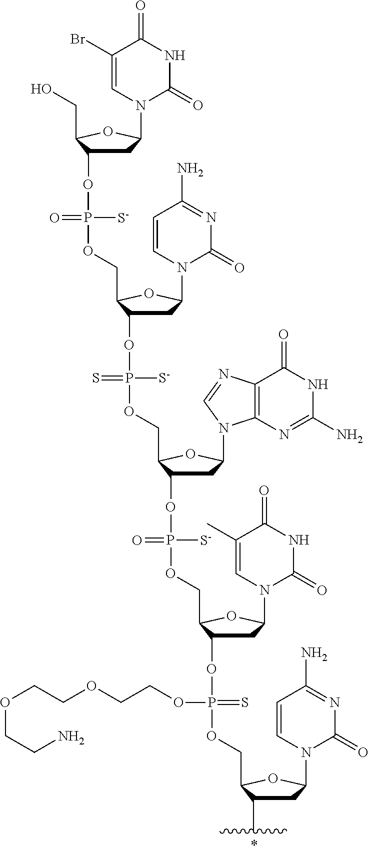 |
| | 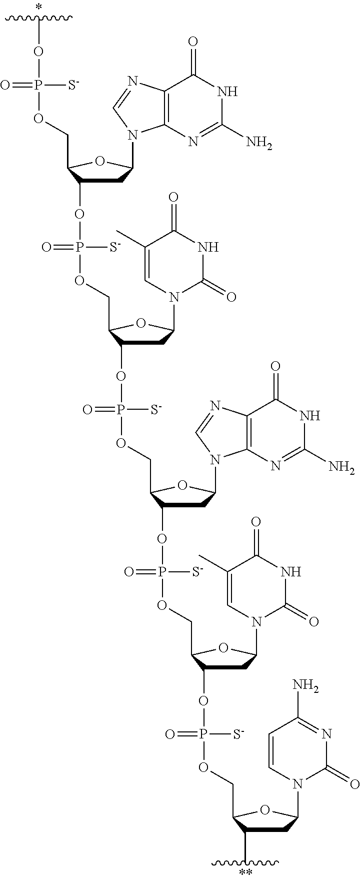 |
| | 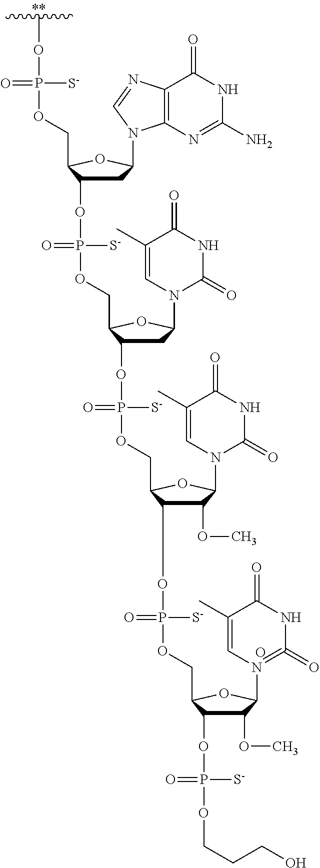 |
| 7.2a | 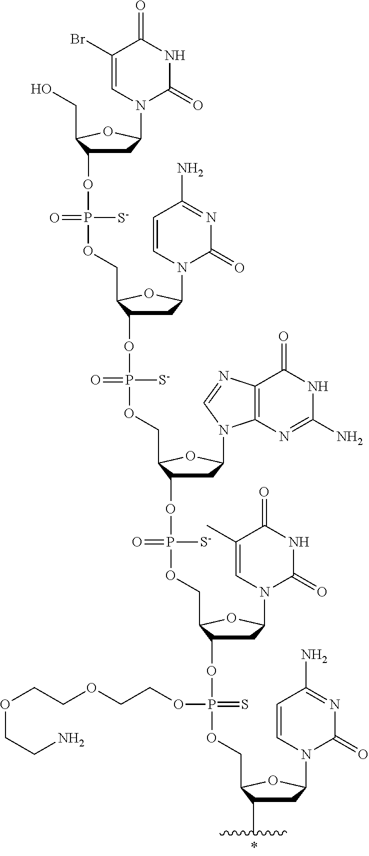 |
| | 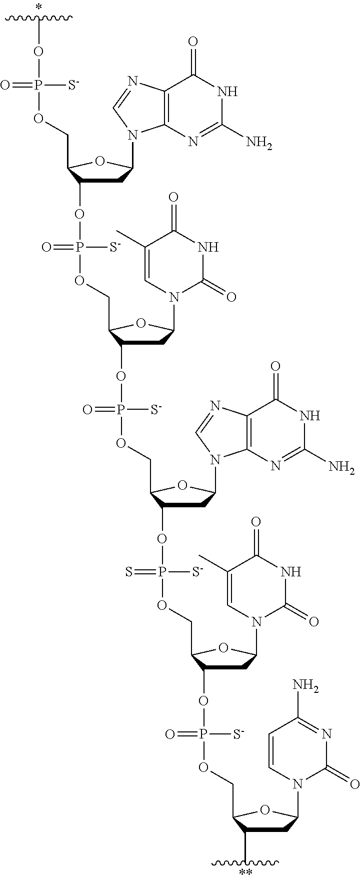 |
| | 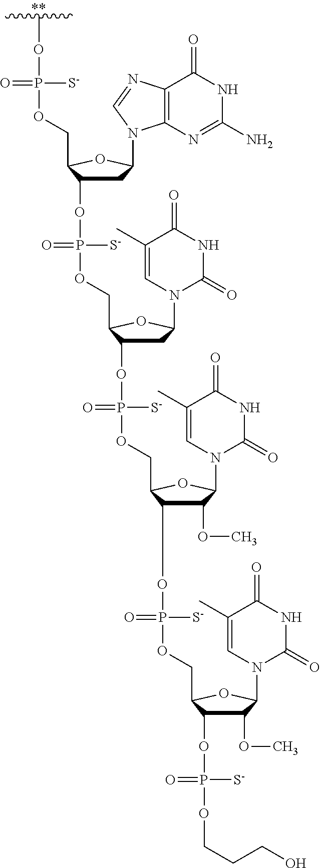 |
| 7.3a | 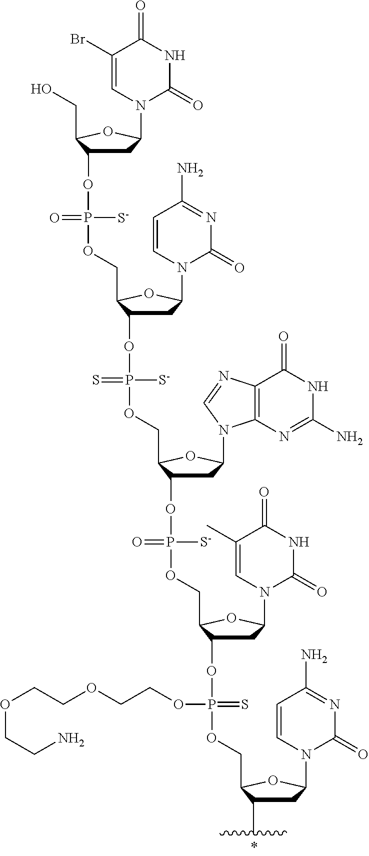 |
| | 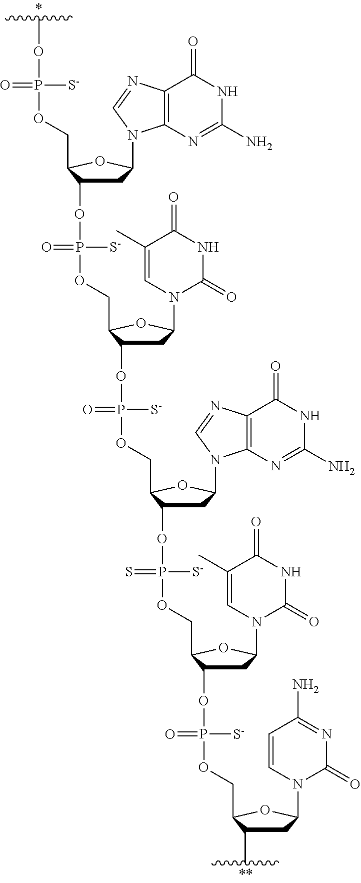 |
| | 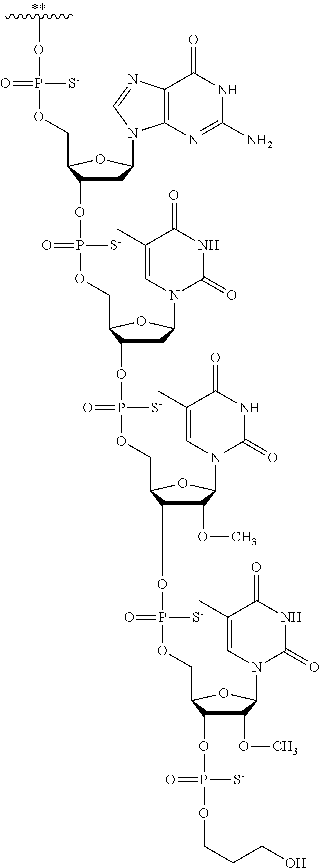 |
| 7.4a | 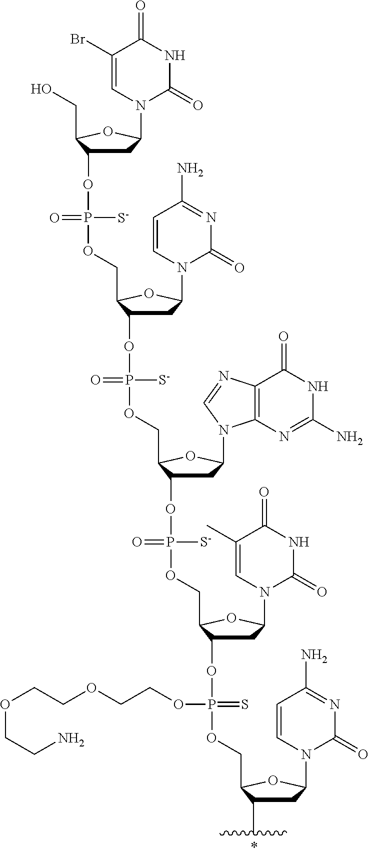 |
| | 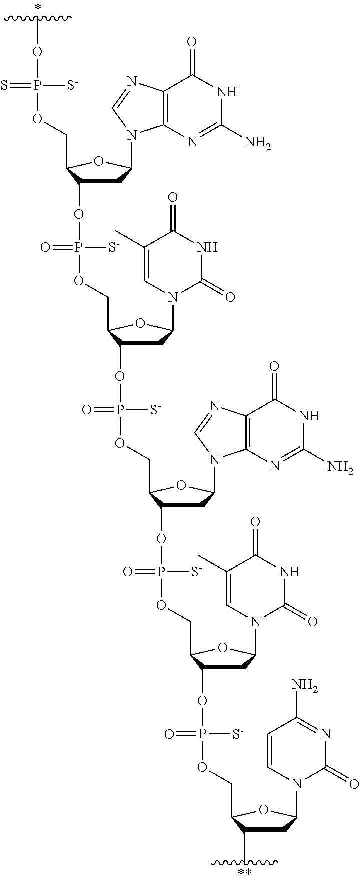 |
| | 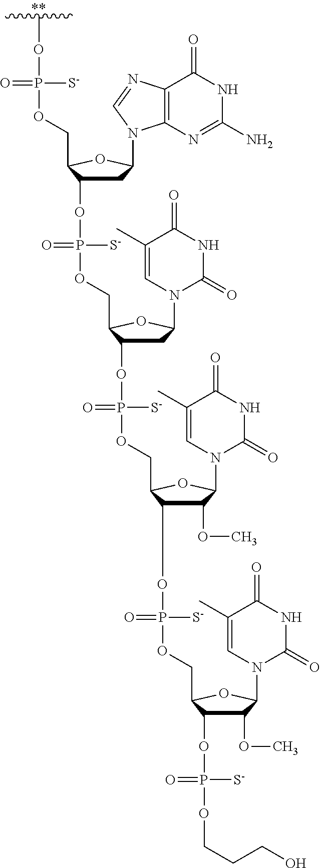 |
| 7.5a | 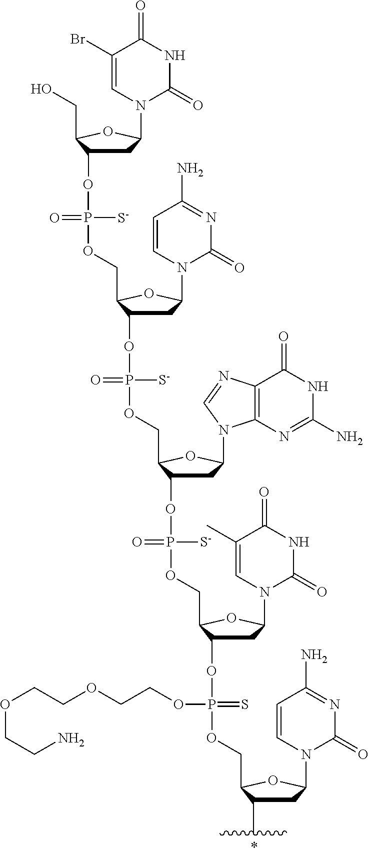 |
| | 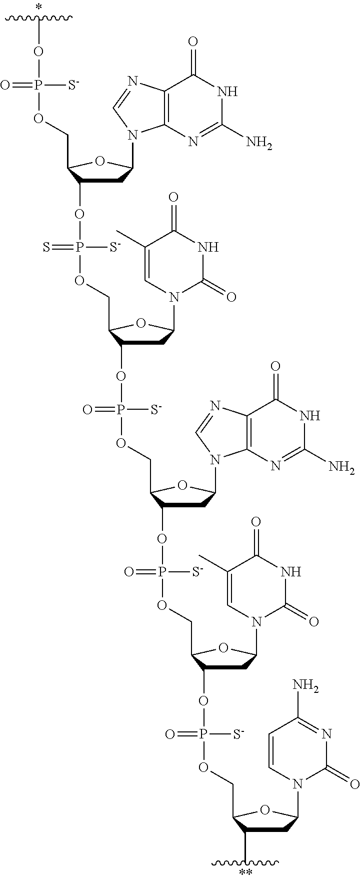 |
| | 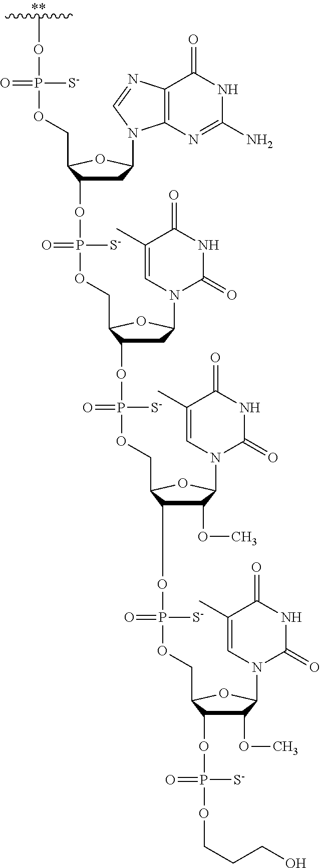 |
| 7.6a | 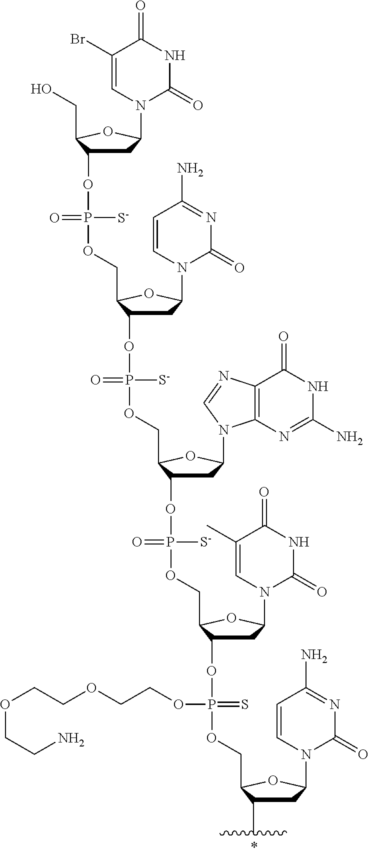 |
| | 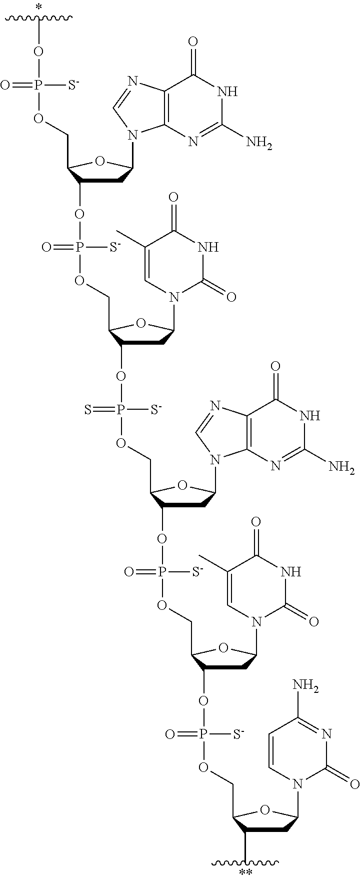 |
| | 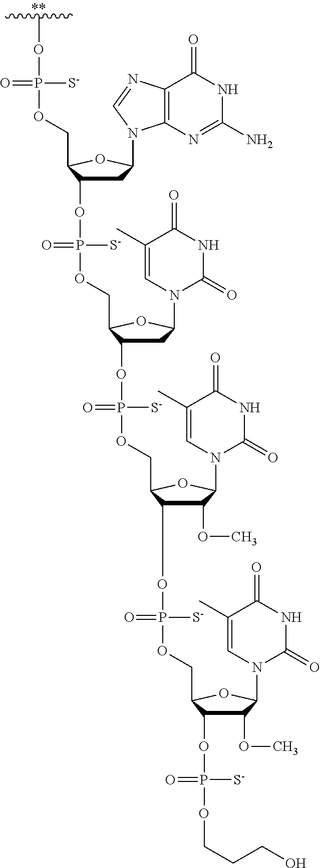 |
| 7.7a | 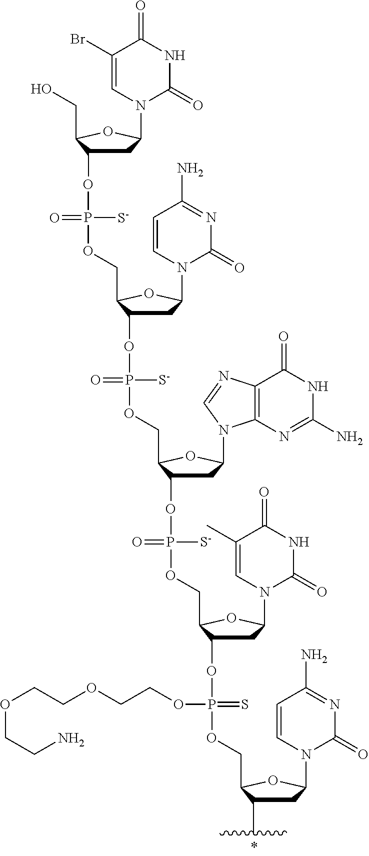 |
| | 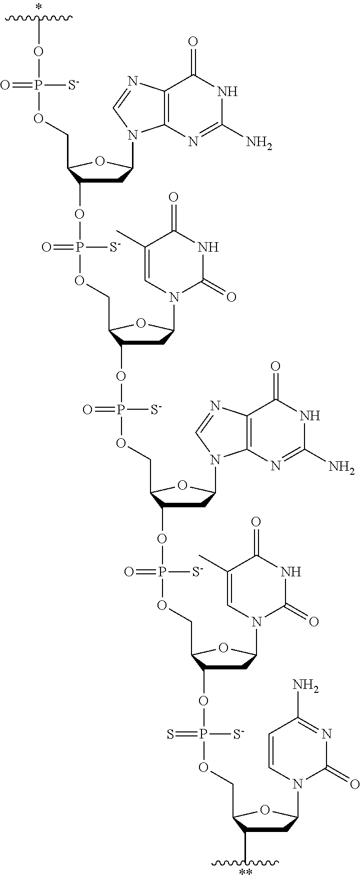 |
| | 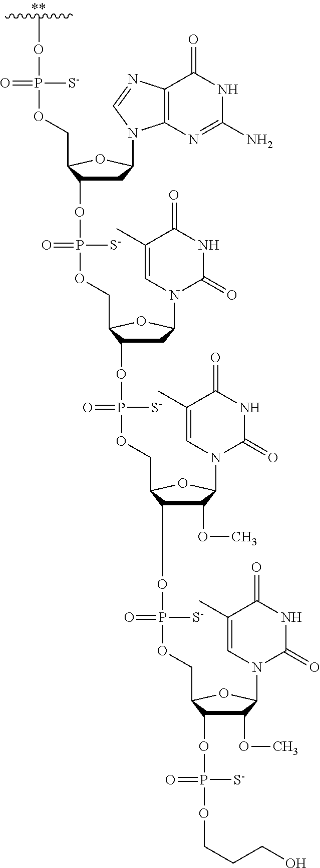 |
| 7.8a | 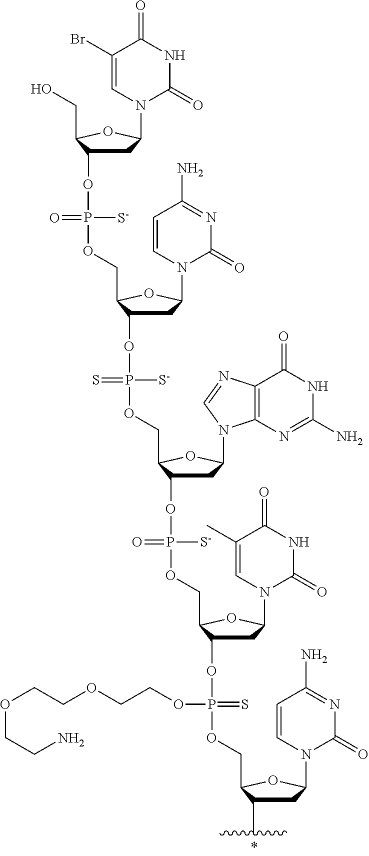 |
| | 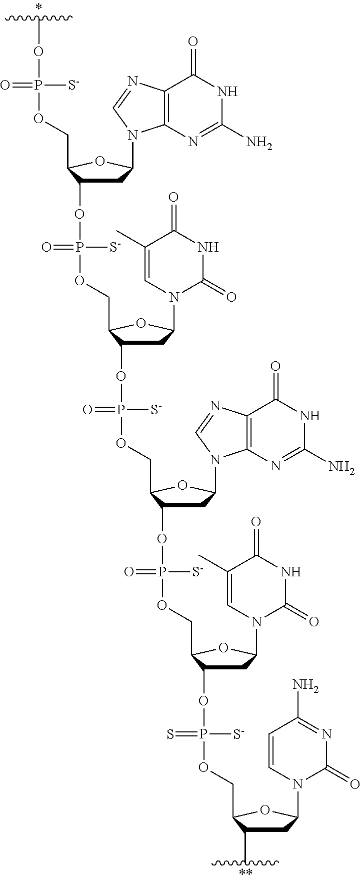 |
| | 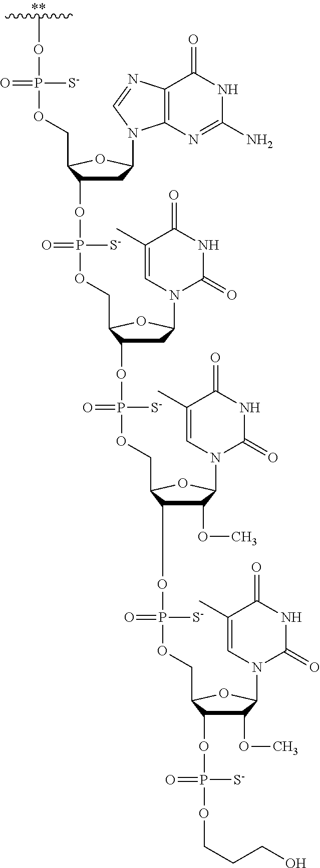 |
| 7.9a | 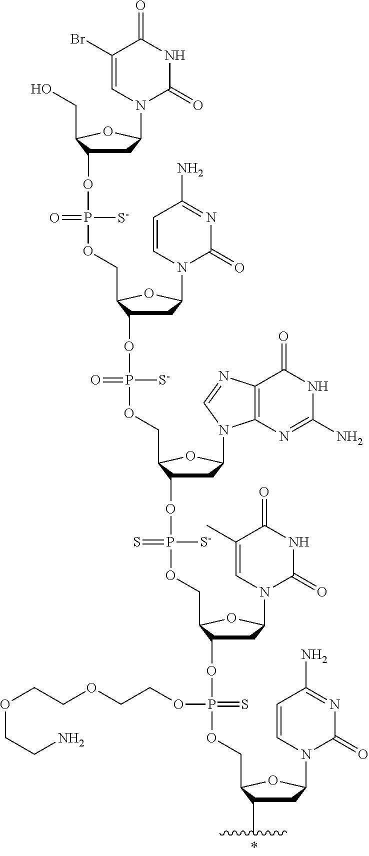 |
| | 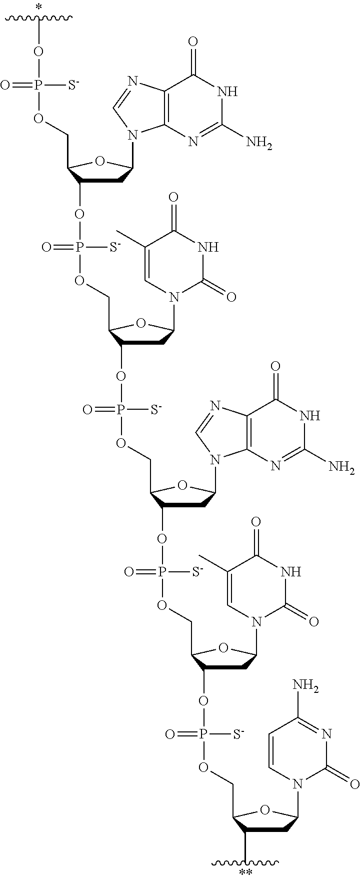 |
| | 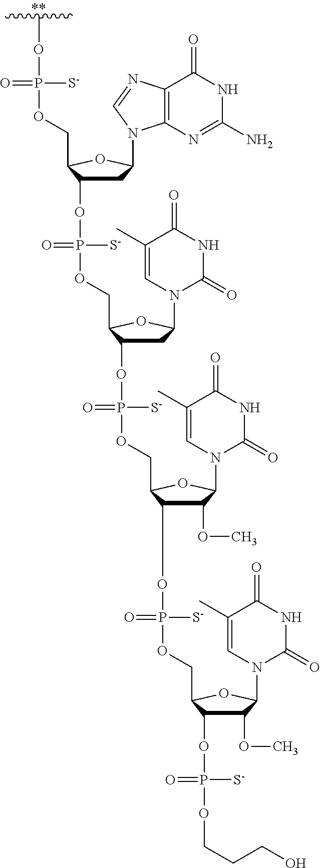 |
| 7.10a | 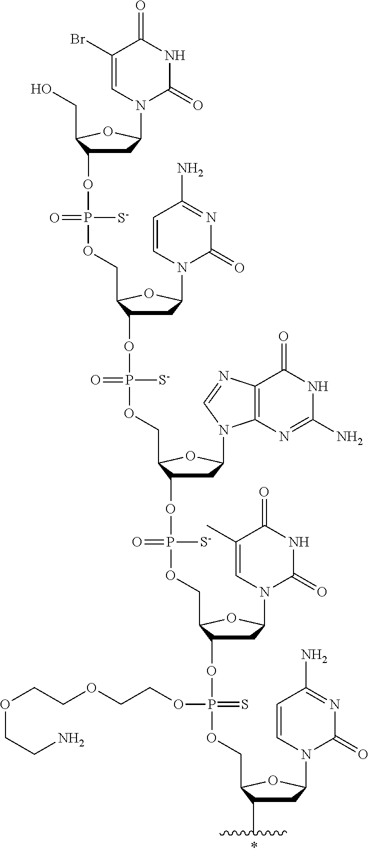 |
| | 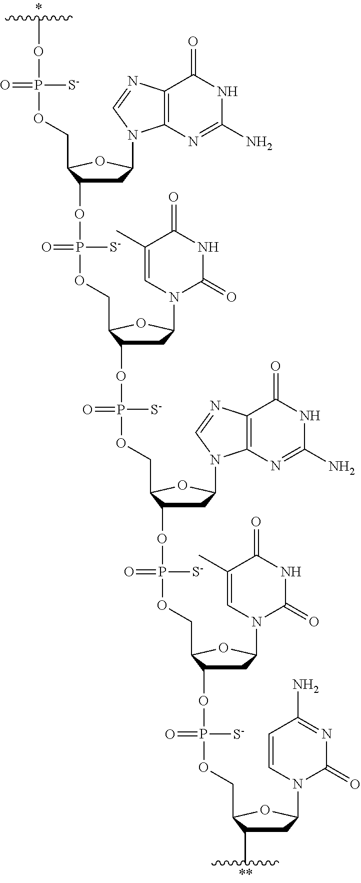 |
| | 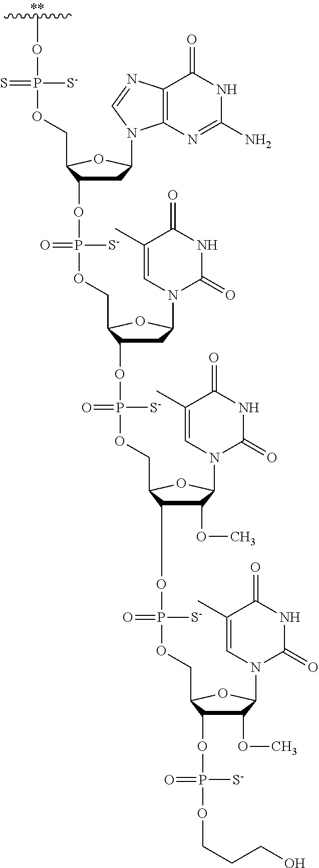 |
| 15.7a | 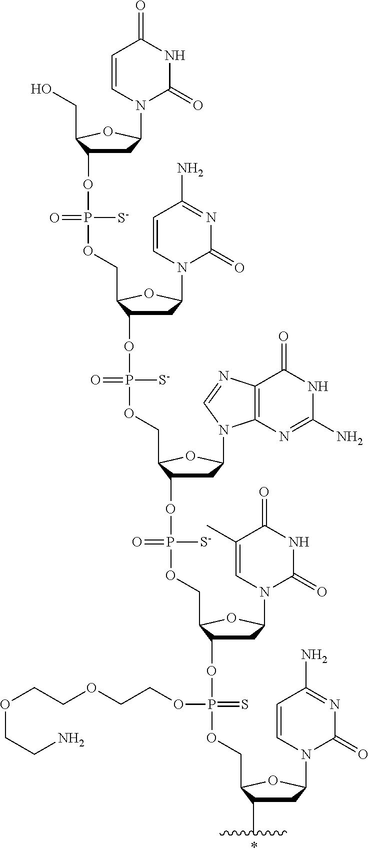 |
| | 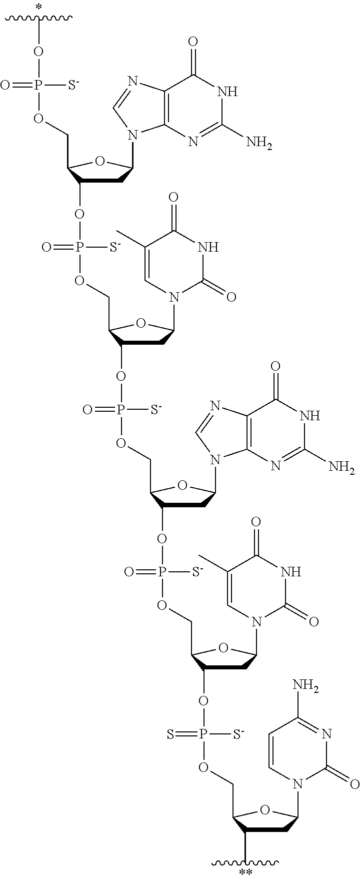 |
| | 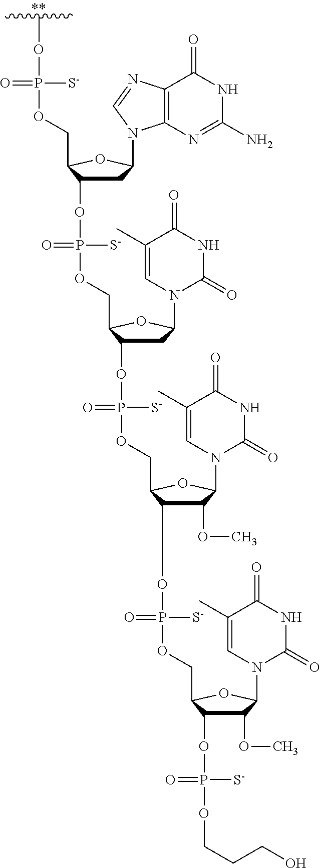 |

In some embodiments, the immunomodulating oligonucleotides of formula (C) may be used as precursors to prepare conjugates comprising a nectin-4 antibody or antigen-binding fragment thereof and one or more immunomodulating oligonucleotides of formula (C) linked via Q-tag as shown in the structures of formula (A) as described herein.

In some embodiments, the immunmodulating oligonucleotides of formula (C) may be modified to attach a linker moiety L to the terminal group T³ in formula (C) to provide immunomodulating oligonucleotides of formula (D). In still another aspect, the immunomodulating oligonucleotides comprises the structure of formula (D),

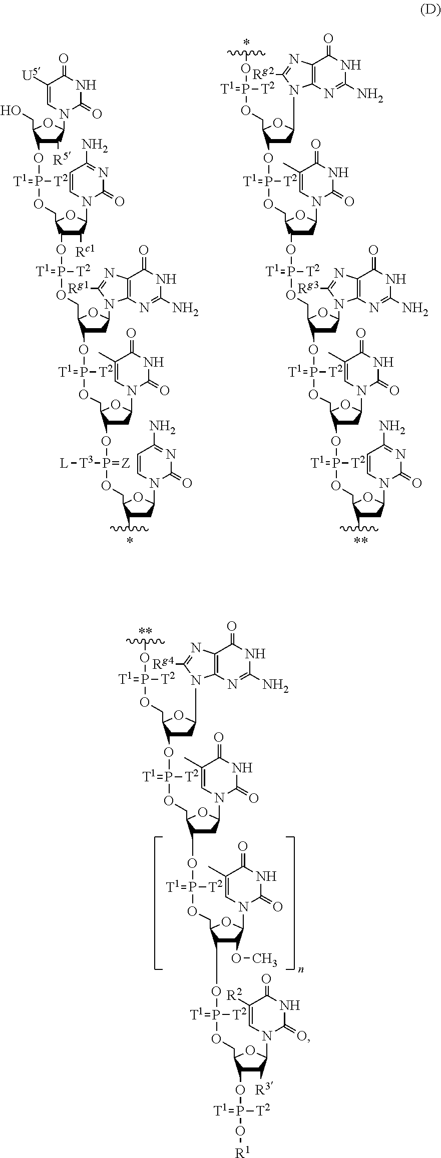

wherein
[symbol] * and [symbol] ** indicate the points of attachment within the oligonucleotide;
each T¹ is independently O or S;
each T² is S⁻;
T³ is a group

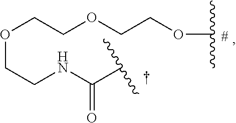 [SMILES: *OCCOCCOCCNC(*)=O]

wherein [symbol] † indicates the point of attachment to L and wherein [symbol] # indicates the point of attachment to the rest of the oligonucleotide;
L is a group

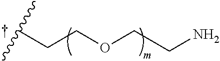

wherein m is an integer from 0 to 50 (such as about 0 to about 10, about 0 to about 30, about 10 to about 30, about 20 to about 30, and values in ranges therebetween) and wherein [symbol] † indicates the point of attachment to the rest of the oligonucleotide via T³;
Z is O or S;
U^{5′} is —H or halogen;
R^{5′} is —H or methoxy;
R^{c1} is —H or methoxy;
R^{g1}, R^{g2}, R^{g3}, and R⁹⁴ are H or oxo, optionally wherein at least one of R^{g1}, R^{g2}, R^{g3}, and R⁹⁴ is oxo and wherein if one of R^{g1}, R^{g2}, R^{g3}, and R⁹⁴ is oxo, then the carbon to which the oxo is attached has a single bond to the ring nitrogen at the 7-position;
R^{3′} is methoxy or 2-methoxyethoxy;
R¹ is —(CH₂)₃—OH;
R² is —H or methyl; and
n is an integer from 0 to 2,
   or a pharmaceutically acceptable salt thereof.

In some embodiments of the present aspect, U^{5′} is —H In other embodiments, U^{5′} is halogen. In certain embodiments, U^{5′} is iodo or bromo. In some embodiments of the present aspect, the immunomodulatory oligonucleotide of formula (D) is an immunomodulatory oligonucleotide of formula (D′). In other embodiments of the present aspect, the immunomodulatory oligonucleotide of formula (D) is an immunomodulatory oligonucleotide of formula (D″).

In some embodiments of the present aspect, the immunomodulatory oligonucleotide comprises the structure of formula (D′),

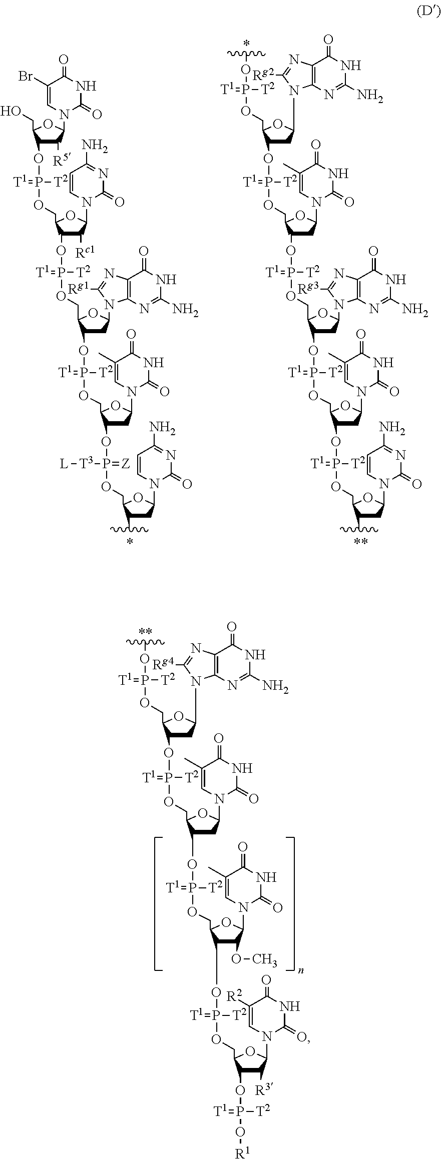

wherein
[symbol] *and [symbol] ** indicate the points of attachment within the oligonucleotide;
each T¹ is independently O or S;
each T² is S⁻;
T³ is a group

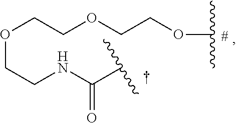 [SMILES: *OCCOCCOCCNC(*)=O]

wherein [symbol] † indicates the point of attachment to L and wherein [symbol] # indicates the point of attachment to the rest of the oligonucleotide;
L is a group

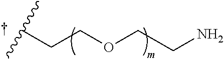

wherein m is an integer from 0 to 50 (such as about 0 to about 10, about 0 to about 30, about 10 to about 30, about 20 to about 30, and values in ranges therebetween) and wherein [symbol] † indicates the point of attachment to the rest of the oligonucleotide via T³;
Z is O or S;
R^{5′} is —H or methoxy;
R^{c1} is —H or methoxy;
R^{g1}, R^{g2}, R^{g3}, and R⁹⁴ are H or oxo, optionally wherein at least one of R^{g1}, R^{g2}, R^{g3}, and R⁹⁴ is oxo and wherein if one of R^{g1}, R^{g2}, R^{g3}, and R⁹⁴ is oxo, then the carbon to which the oxo is attached has a single bond to the ring nitrogen at the 7-position;
R^{3′} is methoxy or 2-methoxyethoxy;
R¹ is —(CH₂)₃—OH;
R² is —H or methyl; and
n is an integer from 0 to 2,
   or a pharmaceutically acceptable salt thereof.

In other embodiments of the present aspect, the immunomodulatory oligonucleotide comprises the structure of formula (D″),

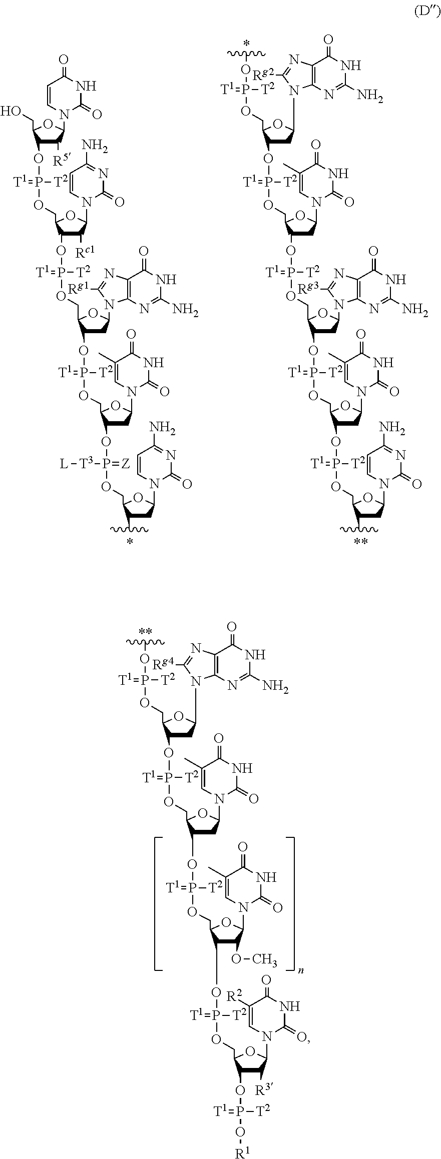

wherein
[symbol] * and [symbol] ** indicate the points of attachment within the oligonucleotide;
each T¹ is independently O or S;
each T² is S⁻;
T³ is a group

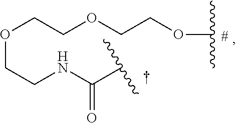 [SMILES: *OCCOCCOCCNC(*)=O]

wherein [symbol] † indicates the point of attachment to L and wherein [symbol] # indicates the point of attachment to the rest of the oligonucleotide;
L is a group

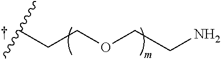

wherein m is an integer from 0 to 50 (such as about 0 to about 10, about 0 to about 30, about 10 to about 30, about 20 to about 30, and values in ranges therebetween) and wherein [symbol] † indicates the point of attachment to the rest of the oligonucleotide via T³;
Z is O or S;
R^{5′} is —H or methoxy;
R^{c1} is —H or methoxy;
R^{g1}, R^{g2}, R^{g3}, and R⁹⁴ are H or oxo, optionally wherein at least one of R^{g1}, R^{g2}, R^{g3}, and R⁹⁴ is oxo and wherein if one of R^{g1}, R^{g2}, R^{g3}, and R⁹⁴ is oxo, then the carbon to which the oxo is attached has a single bond to the ring nitrogen at the 7-position;
R^{3′} is methoxy or 2-methoxyethoxy;
R¹ is —(CH₂)₃—OH;
R² is —H or methyl; and
n is an integer from 0 to 2,
   or a pharmaceutically acceptable salt thereof.

In some embodiments of the present aspect, the immunomodulating oligonucleotide comprises the structure of formula (D′),

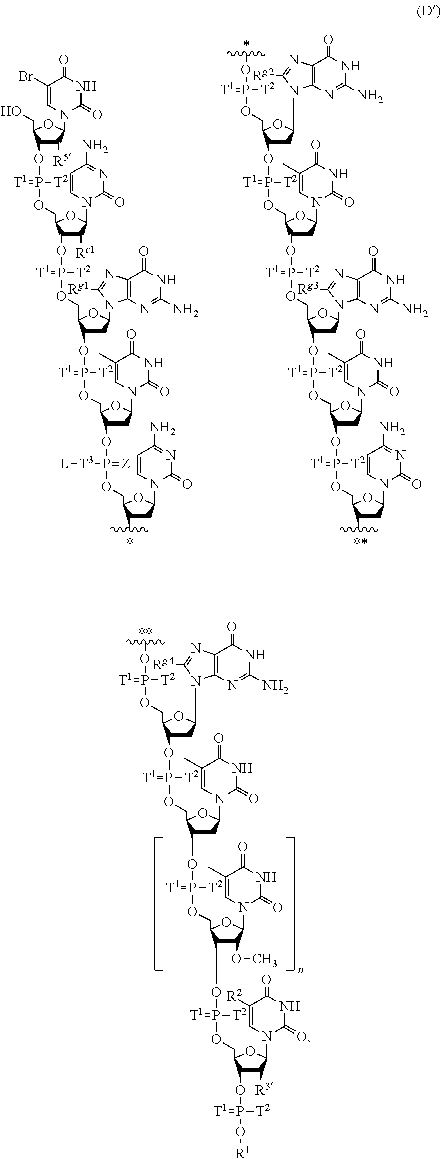

wherein
[symbol] * and [symbol] ** indicate the points of attachment within the oligonucleotide;
each T¹ is independently O or S;
each T² is S⁻;
T³ is a group

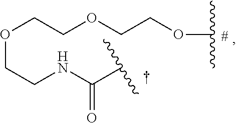 [SMILES: *OCCOCCOCCNC(*)=O]

wherein [symbol] † indicates the point of attachment to L and wherein [symbol] # indicates the point of attachment to the rest of the oligonucleotide;
L is a group

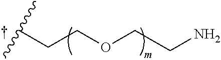

wherein m is an integer from 0 to 50 (such as about 0 to about 10, about 0 to about 30, about 10 to about 30, about 20 to about 30, and values in ranges therebetween) and wherein [symbol] † indicates the point of attachment to the rest of the oligonucleotide via T³;
Z is O or S;
R^{5′} is —H or methoxy;
R¹ is —H or methoxy;
R^{g1}, R^{g2}, R^{g3}, and R^{g4} are H;
R^{3′} is methoxy;
R¹ is —(CH₂)₃—OH;
R² is —H or methyl; and
n is an integer from 0 to 2,
   or a pharmaceutically acceptable salt thereof.

In still other embodiments of the present aspect, the immunomodulatory oligonucleotide comprises the structure of formula (D″),

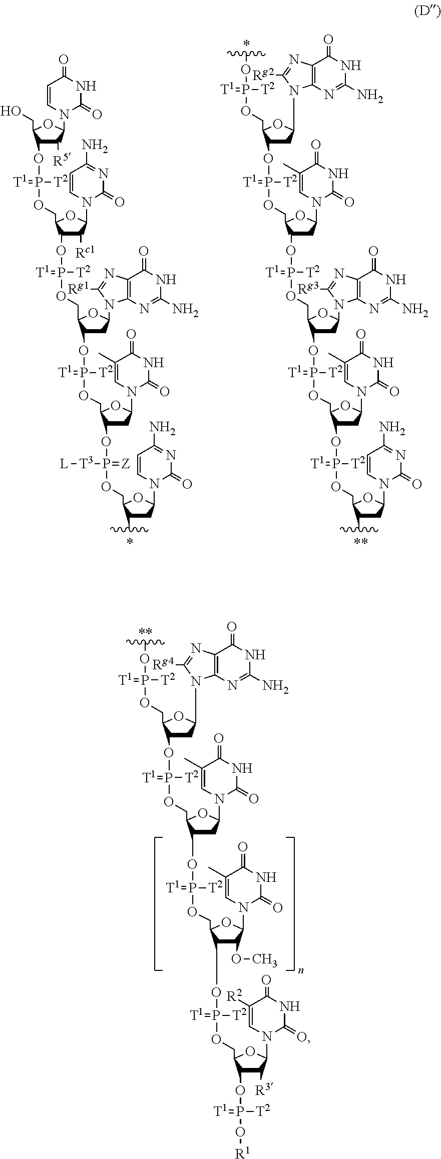

wherein
[symbol] * and [symbol] ** indicate the points of attachment within the oligonucleotide;
each T¹ is independently O or S;
each T² is S⁻;
T³ is a group

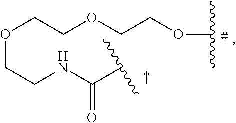 [SMILES: *OCCOCCOCCNC(*)=O]

wherein [symbol] † indicates the point of attachment to L and wherein [symbol] # indicates the point of attachment to the rest of the oligonucleotide;
L is a group

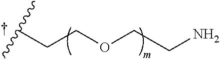

wherein m is an integer from 0 to 50 (such as about 0 to about 10, about 0 to about 30, about 10 to about 30, about 20 to about 30, and values in ranges therebetween) and wherein [symbol] † indicates the point of attachment to the rest of the oligonucleotide via T³;
Z is O or S;
R^{5′} is —H or methoxy;
R¹ is —H or methoxy;
R^{g1}, R^{g2}, R^{g3}, and R^{g4} are H;
R^{3′} is methoxy;
R¹ is —(CH₂)₃—OH;
R² is —H or methyl; and
n is an integer from 0 to 2,
   or a pharmaceutically acceptable salt thereof.

In some embodiments of the present aspect, Z is S. In additional embodiments, the oligonucleotide comprises at least one pair of geminal T¹ and T² wherein T¹ is S and T² is S⁻. In certain embodiments, the oligonucleotide comprises at least two pairs of geminal T¹ and T² wherein T¹ is S and T² is S⁻.

In still yet another embodiment of the present aspect, the oligonucleotide comprises the structure of formula (D),

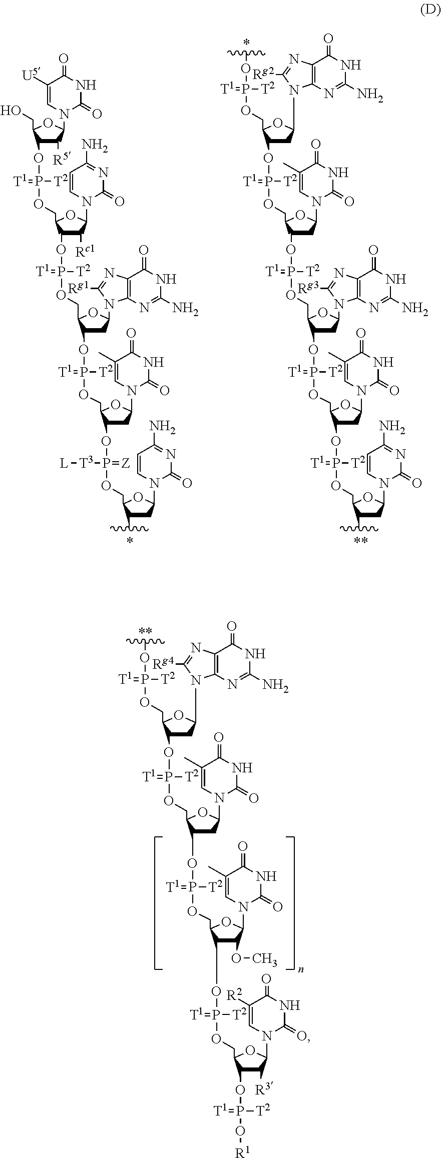

wherein [symbol] * and [symbol] ** indicate the points of attachment within the oligonucleotide;
each T¹ is independently O or S;
each T² is S⁻;
provided that the oligonucleotide comprises at least one pair of geminal T¹ and T² wherein T¹ is S and T² is 5,
T³ is a group

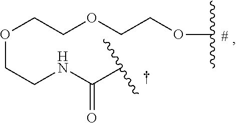 [SMILES: *OCCOCCOCCNC(*)=O]

wherein [symbol] † indicates the point of attachment to L and wherein [symbol] # indicates the point of attachment to the rest of the oligonucleotide;
L is a group

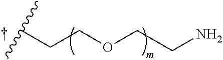

wherein m is an integer from 0 to 50 (such as about 0 to about 10, about 0 to about 30, about 10 to about 30, about 20 to about 30, and values in ranges therebetween) and wherein [symbol] † indicates the point of attachment to the rest of the oligonucleotide via T³;
Z is O or S;
U^{5′} is —H or halogen;
R^{5′} is —H;
R^{c1} is —H;
R^{g1}, R^{g2}, R^{g3}, and R^{g4} are H;
R^{3′} is methoxy;
R¹ is —(CH₂)₃—OH;
R² is -methyl; and
n is 1,
or a pharmaceutically acceptable salt thereof.

In some embodiments of any of the foregoing, the at least one pair of geminal T¹ and T² wherein T¹ is S and T² is S is between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11. In still other embodiments of the foregoing, the oligonucleotide comprises at least two pairs of of geminal T¹ and T² wherein T¹ is S and T² is S, and wherein the at least two pairs of of geminal T¹ and T² wherein T¹ is S and T² is S are between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11.

In some embodiments, the oligonucleotide comprises one or two pairs of geminal T¹ and T² wherein T¹ is S and T² is S, and wherein the one or two pairs of geminal T¹ and T² are between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11. In certain embodiments, the oligonucleotide comprises one pair of geminal T¹ and T² wherein T¹ is S and T² is S, and wherein the pair of geminal T¹ and T² is between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11. In certain other embodiments, the oligonucleotide comprises two pairs of geminal T¹ and T² wherein T¹ is S and T² is S, and wherein the two pairs of geminal T¹ and T² wherein T¹ is S and T² is S are between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11.

In some embodiments, R^{5′} is H. In other embodiments, R^{5′} is methoxy. In some embodiments, R^{c1} is H. In yet other embodiments, R^{c1} is methoxy. In still further embodiments, R² is methyl. In still other embodiments, R² is H. In yet other additional embodiments, which may be combined with any of the preceding embodiments, T³ is

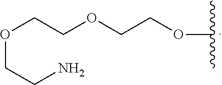 [SMILES: *OCCOCCOCCN]

In still other embodiments, T³ is

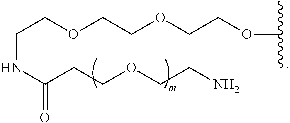

In certain embodiments, m is an integer from 20 to 25 (such as any of 20, 21, 22, 23, 24, and 25).

In another aspect, the immunomodulating oligonucleotide of formula (D) is an oligonucleotide selected from the group consisting of the oligonucleotides of Table 5 and Table 6, or a pharmaceutically acceptable salt thereof. In still further embodiments of the present aspect, the oligonucleotide of formula (D) is selected from the group consisting of the oligonucleotides of Table 6, or a pharmaceutically acceptable salt thereof.

TABLE 5
Modified Oligonucleotide Structures (with —PEG₃NHCOPEG₂₄NH₂)

| Cmpd | |
|---|---|
| # | Structure |
| 1.1b | 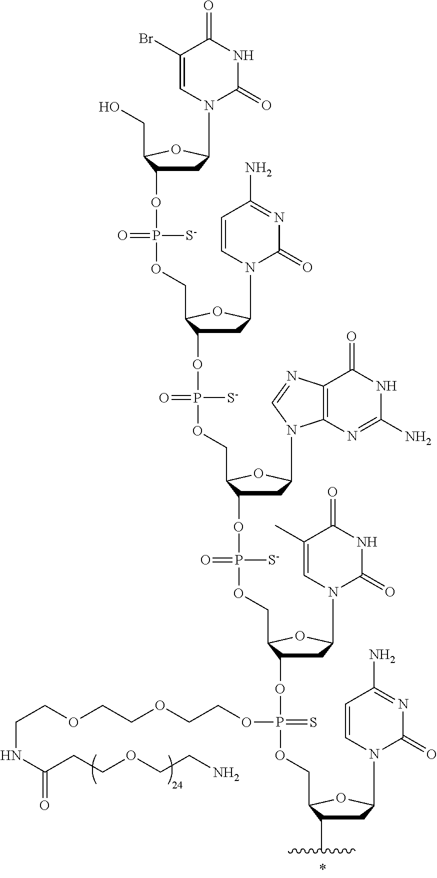 |
| | 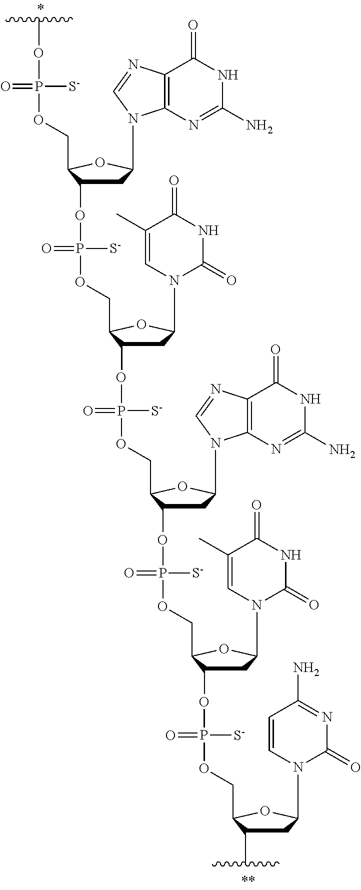 |
| | 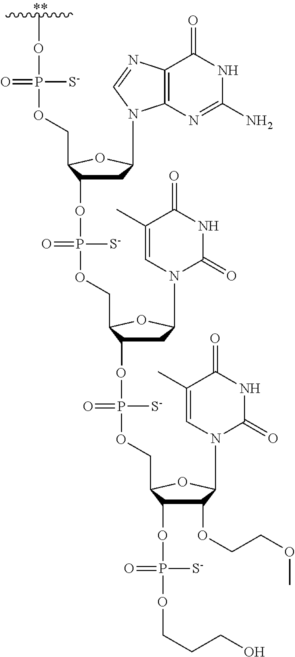 |
| 2.1b | 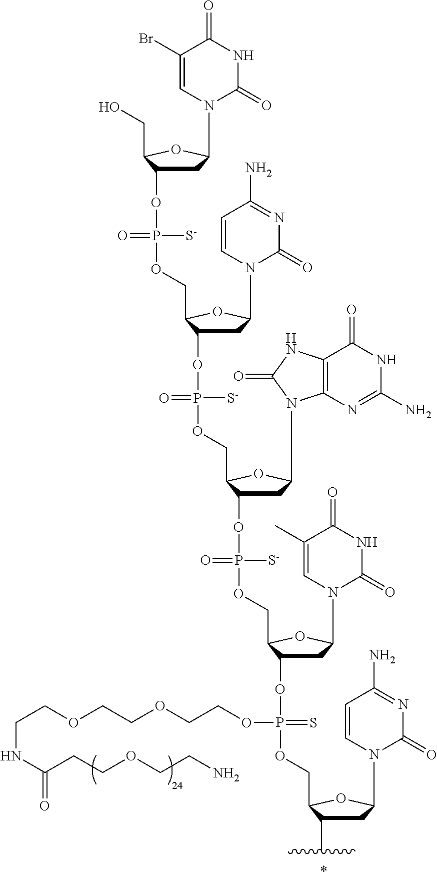 |
| | 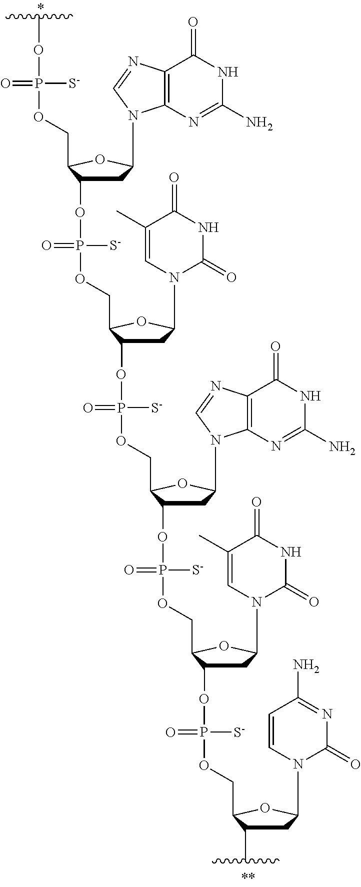 |
| | 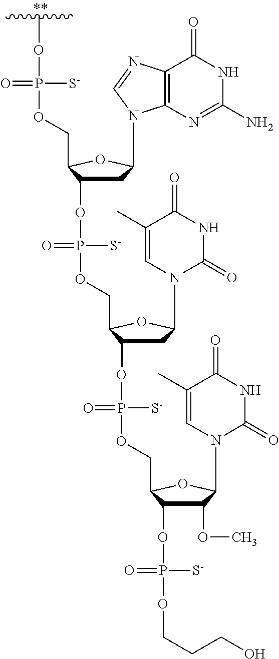 |
| 2.2b | 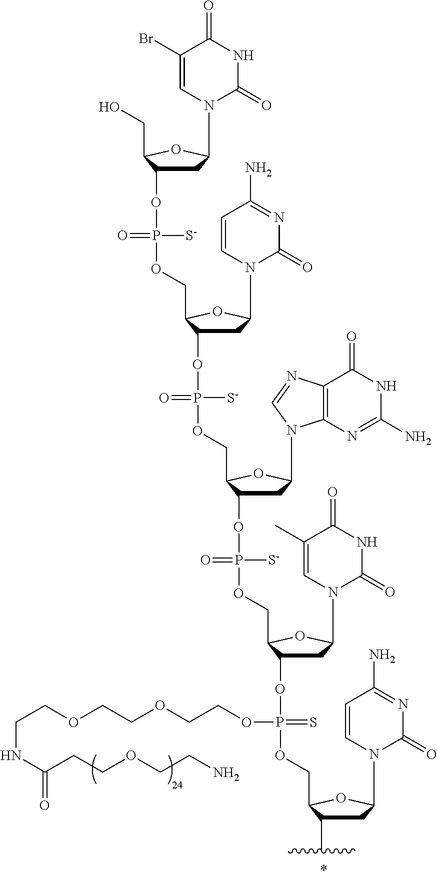 |
| | 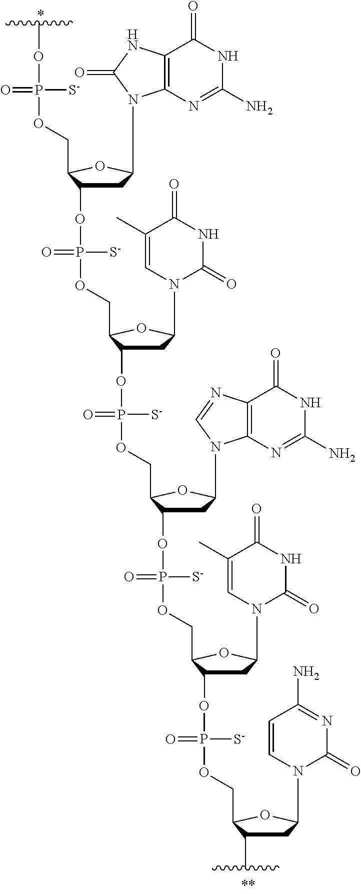 |
| | 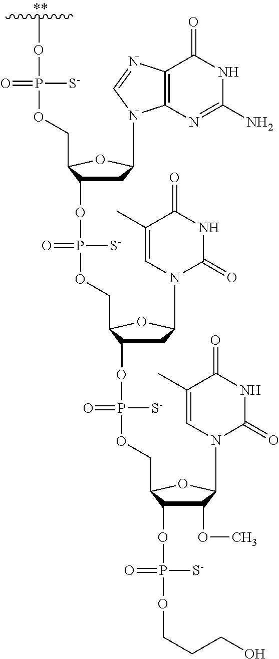 |
| 2.3b | 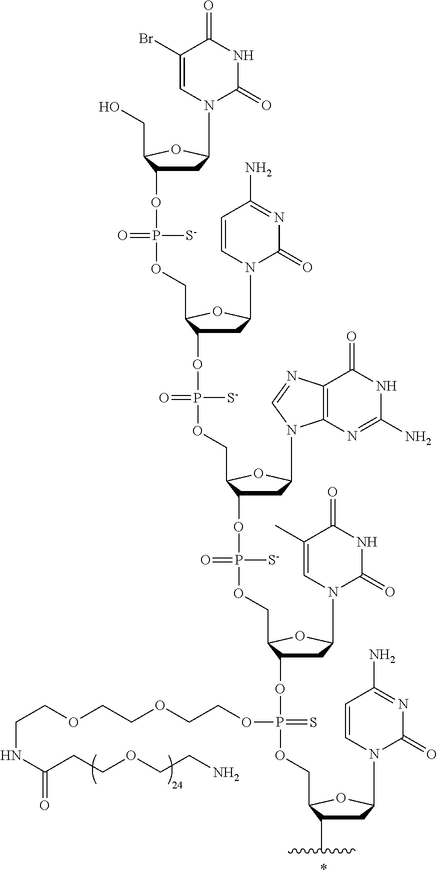 |
| | 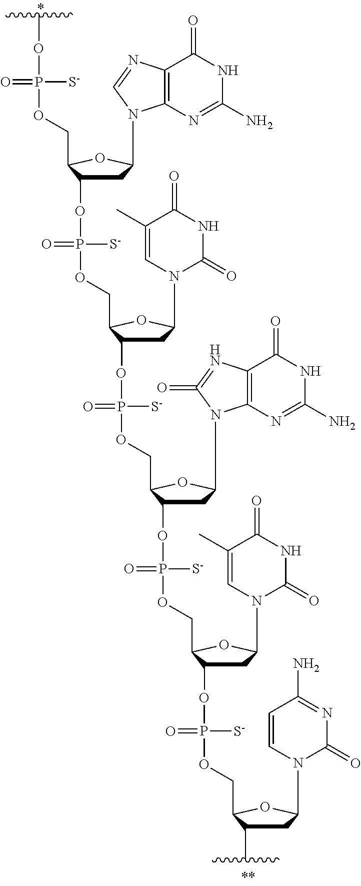 |
| | 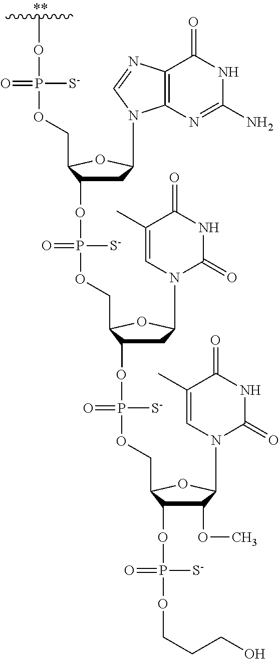 |
| 2.4b | 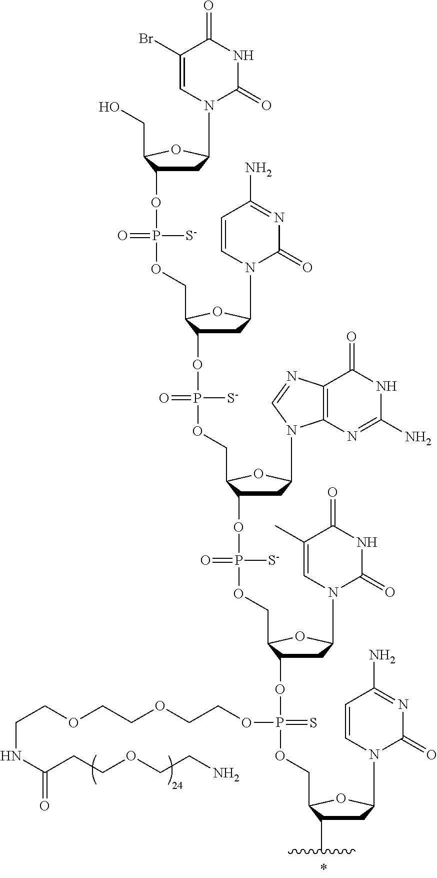 |
| | 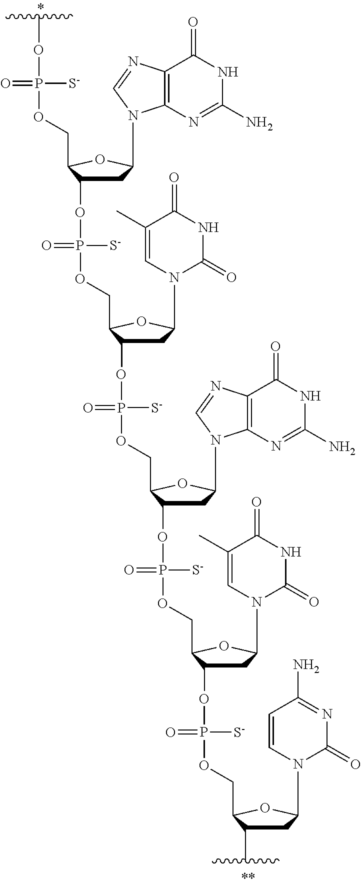 |
| | 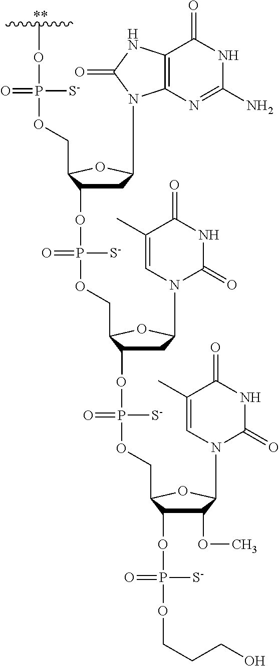 |

TABLE 6
Modified Oligonucleotide Structures (with —PEG₃NHCOPEG₂₄NH₂)

| Cmpd | |
|---|---|
| # | Structure |
| 3.1b | 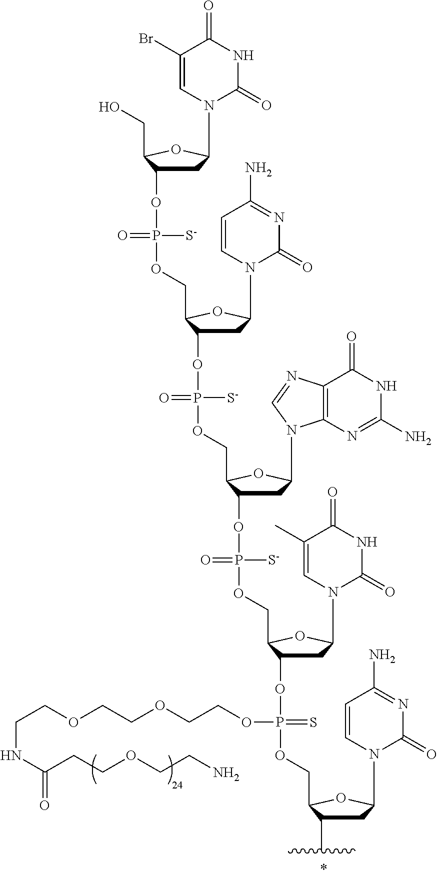 |
| | 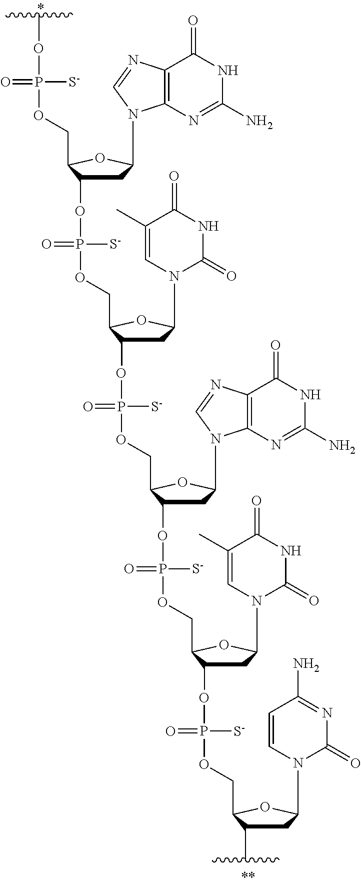 |
| | 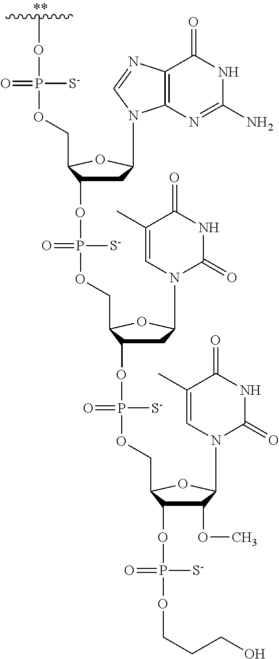 |
| 3.2b | 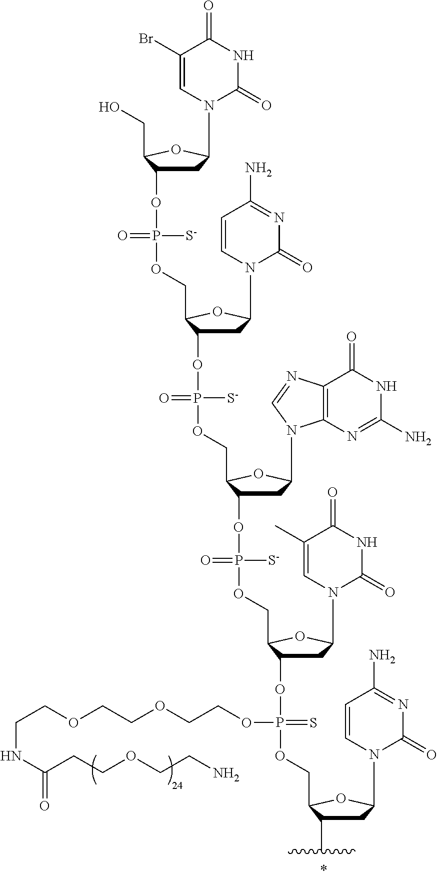 |
| | 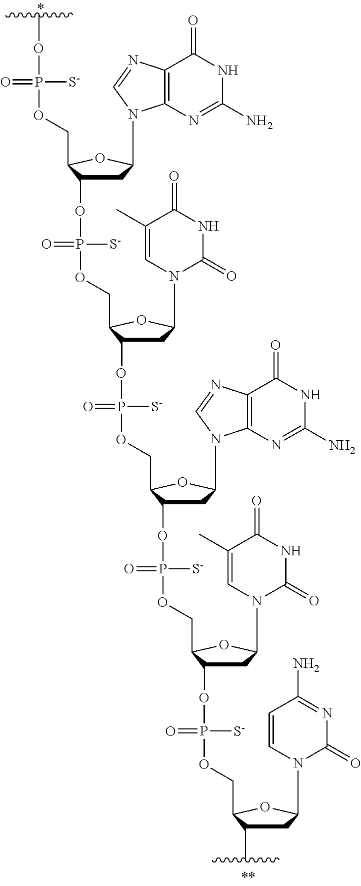 |
| | 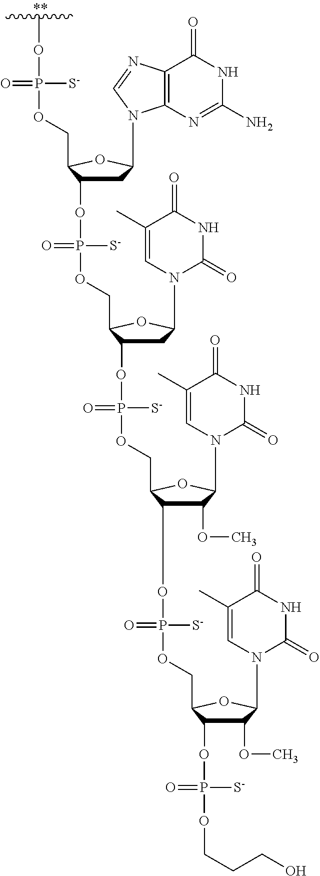 |
| 3.3b | 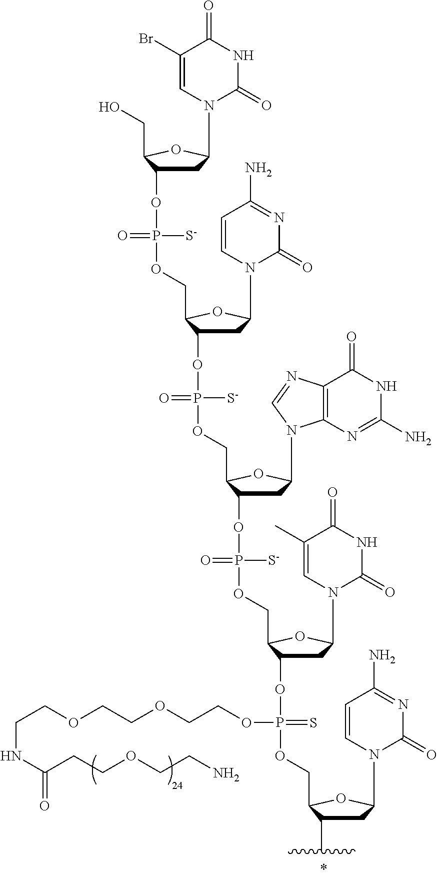 |
| | 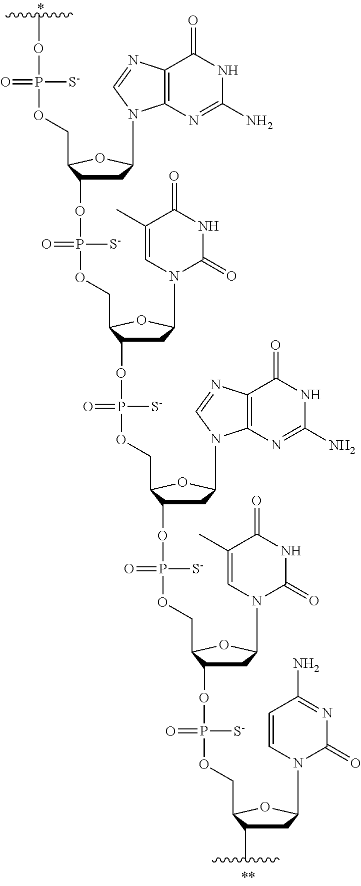 |
| | 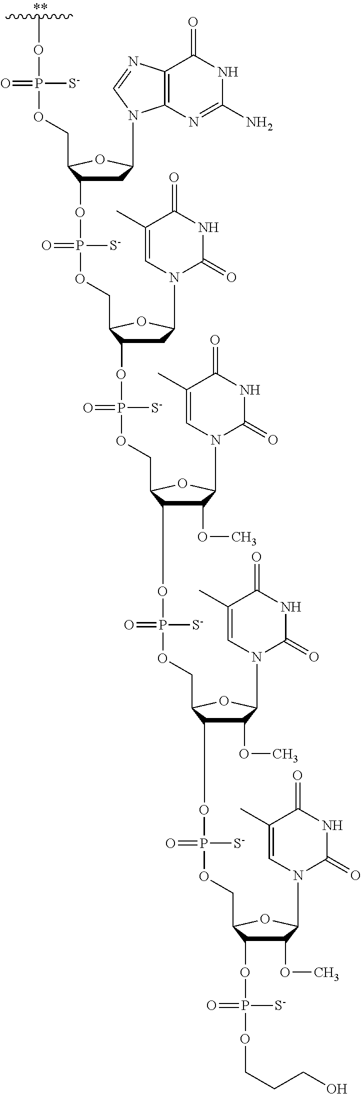 |
| 4.1b | 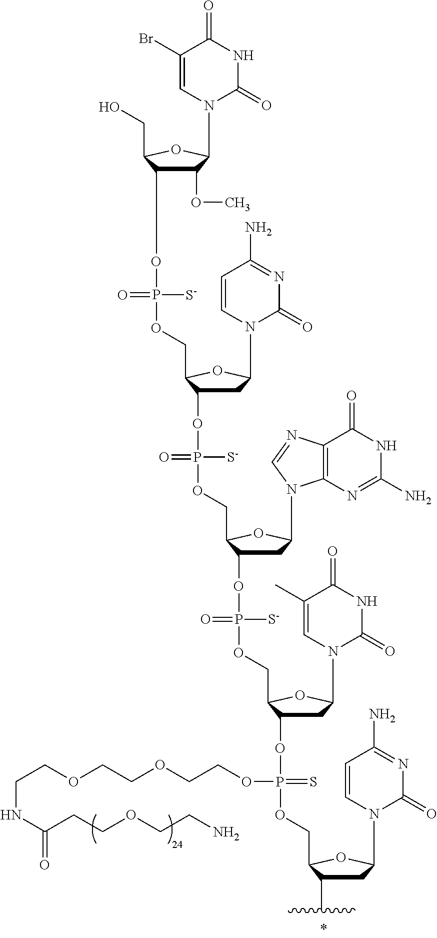 |
| | 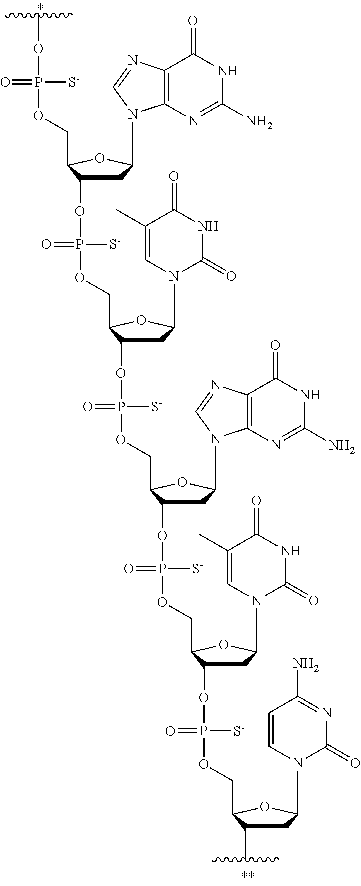 |
| | 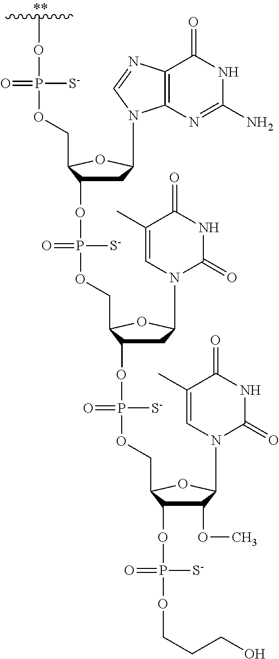 |
| 4.2b | 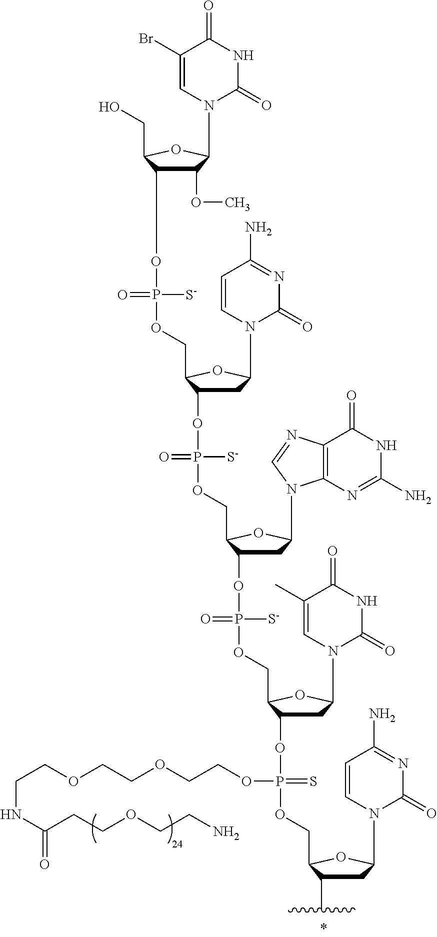 |
| | 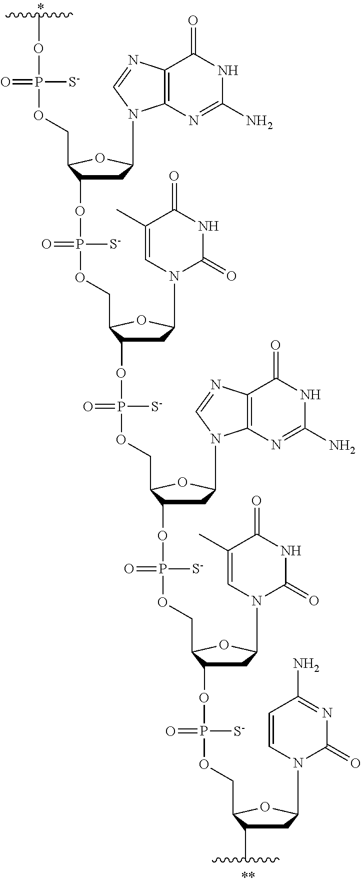 |
| | 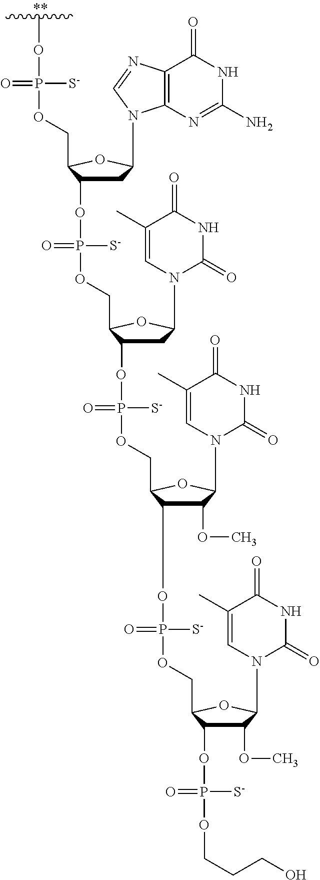 |
| 4.3b | 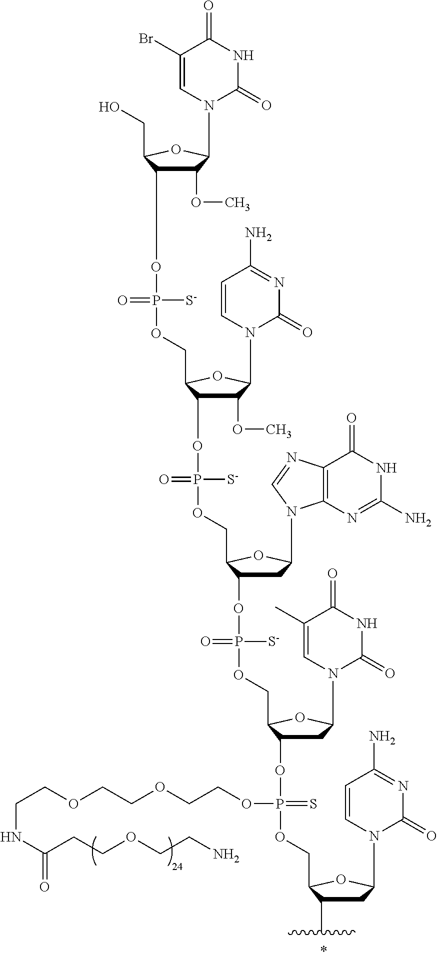 |
| | 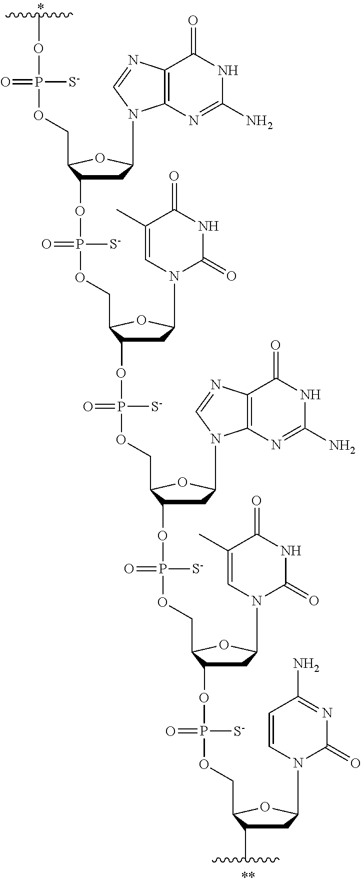 |
| | 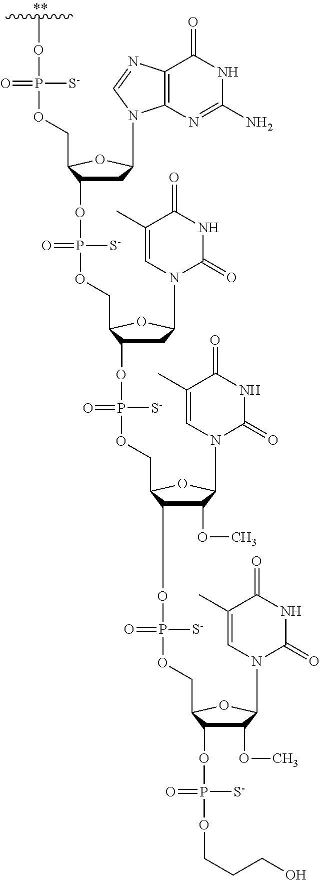 |
| 5.1b | 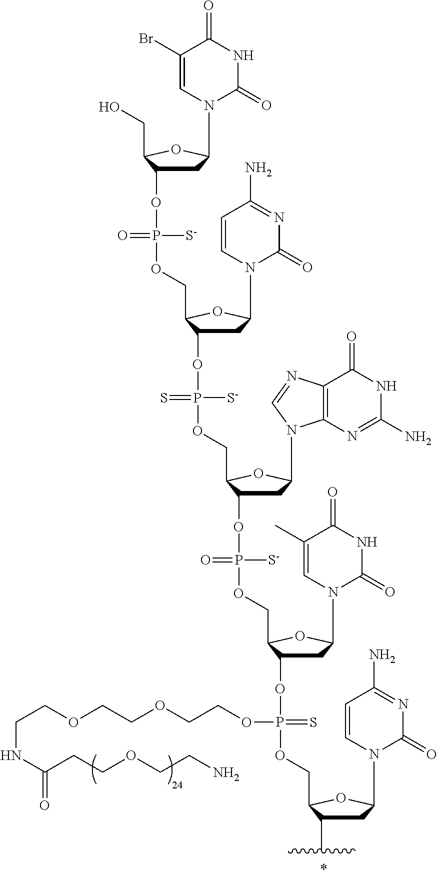 |
| | 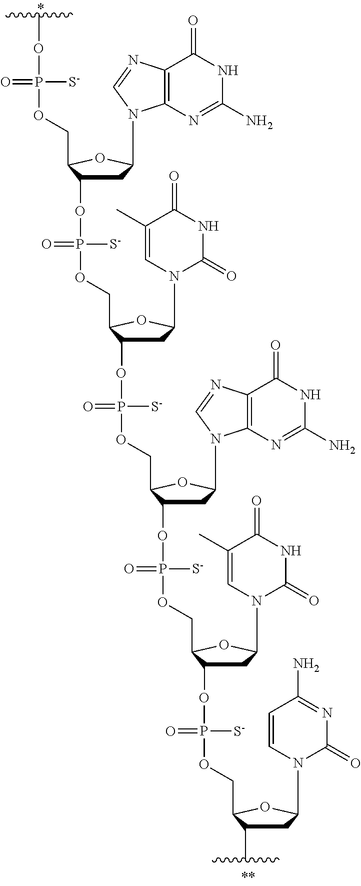 |
| | 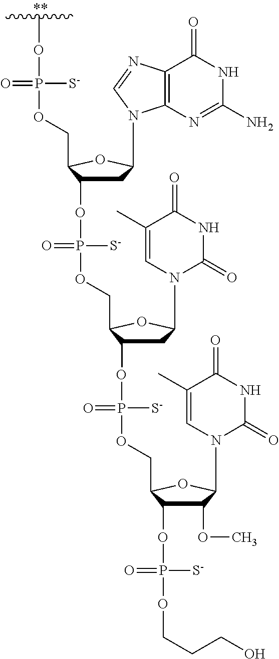 |
| 5.2b | 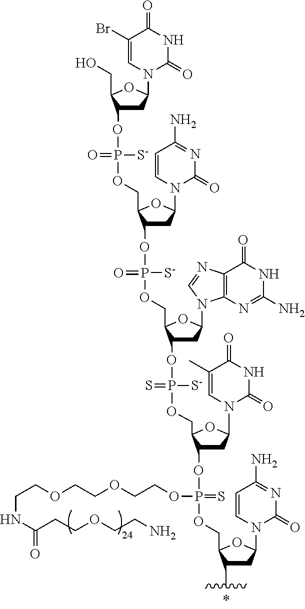 |
| | 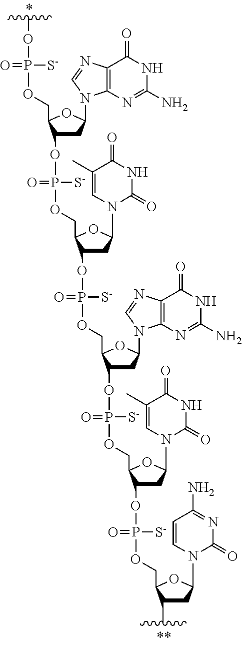 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 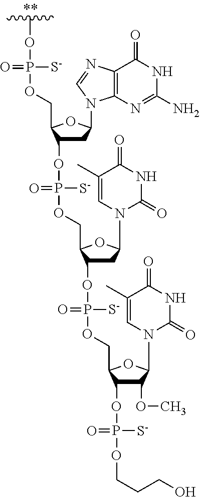 |
| 5.3b | 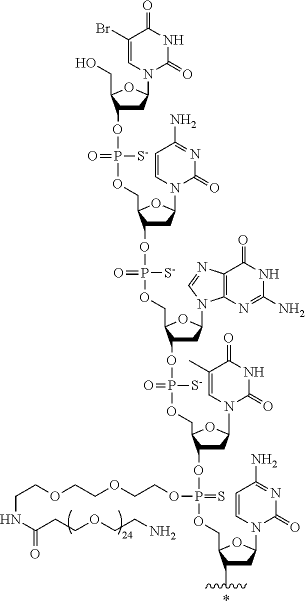 |
| | 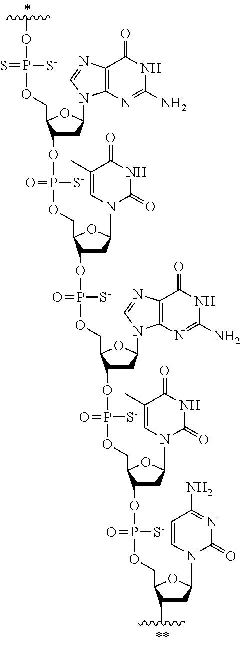 [SMILES: *OP(=S)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 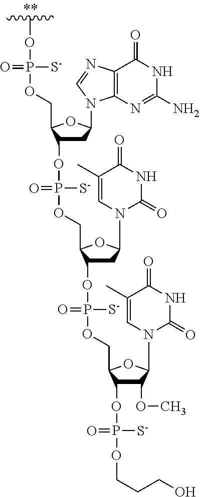 |
| 5.4b | 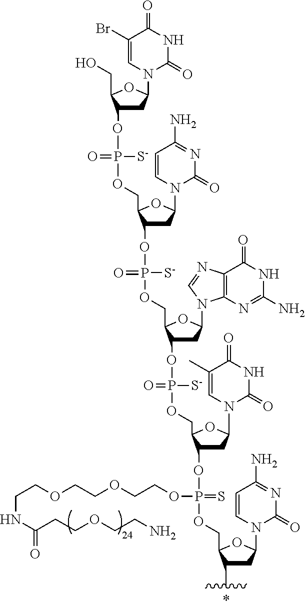 |
| | 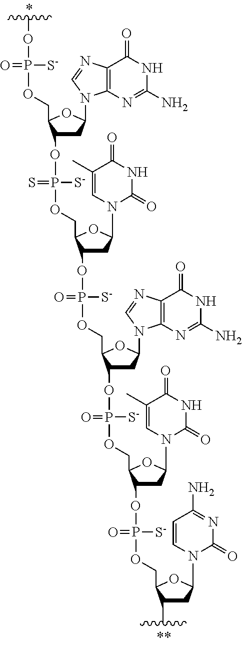 [SMILES: *OP(=O)([S-])OC[C@H]1O[C@@H](n2cnc3c(=O)[nH]c(N)nc32)C[C@H]1OP(=S)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 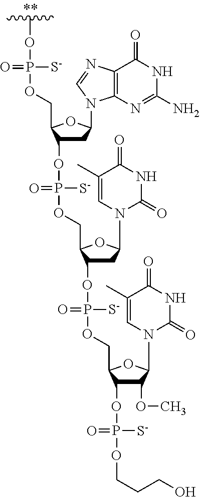 |
| 5.5b | 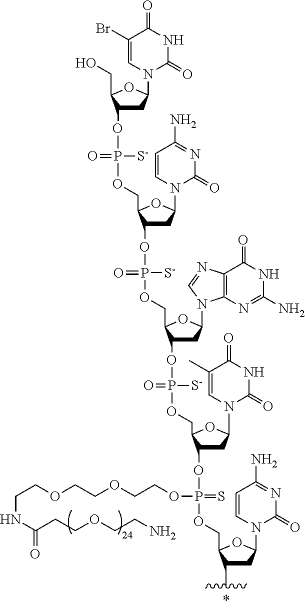 |
| | 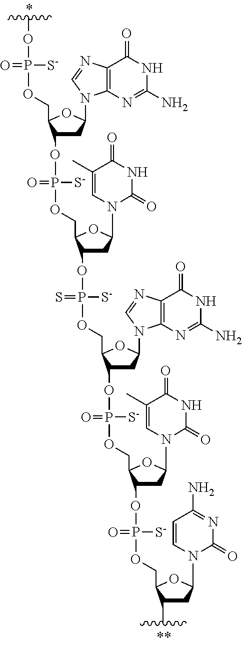 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=S)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 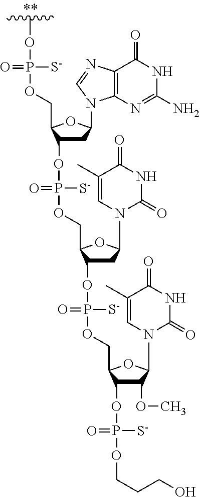 |
| 5.6b | 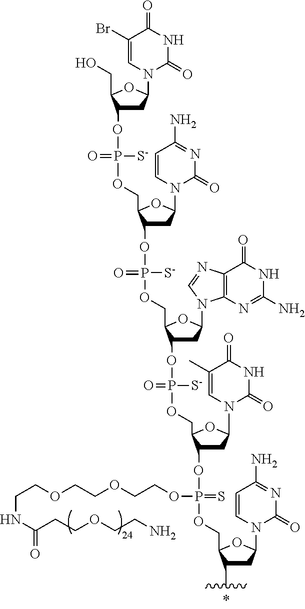 |
| | 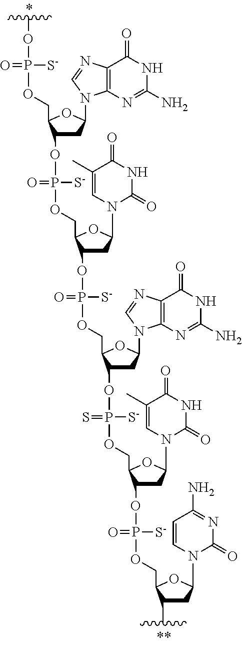 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=S)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 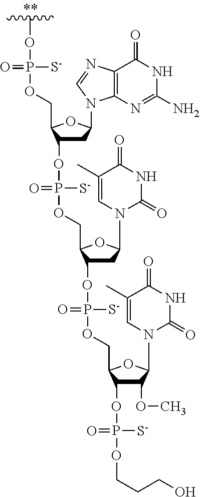 |
| 5.7b | 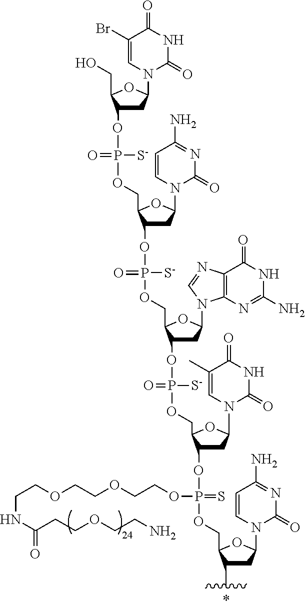 |
| | 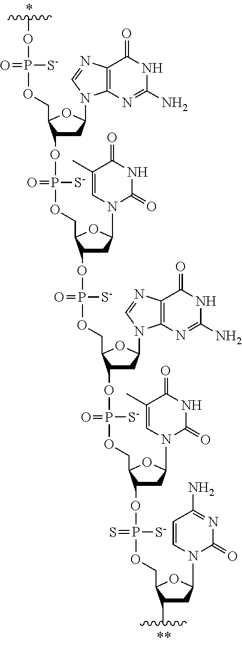 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=S)([S-])OC[C@H]1O[C@@H](n2ccc(N)nc2=O)C[C@H]1*] |
| | 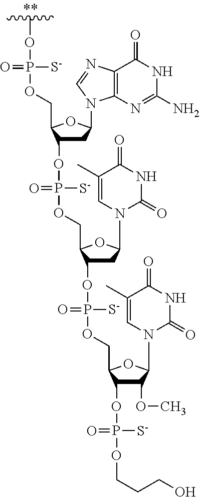 |
| 5.8b | 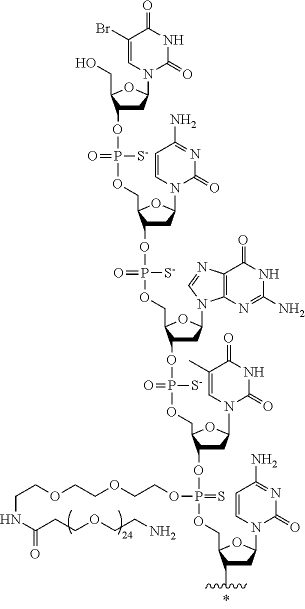 |
| | 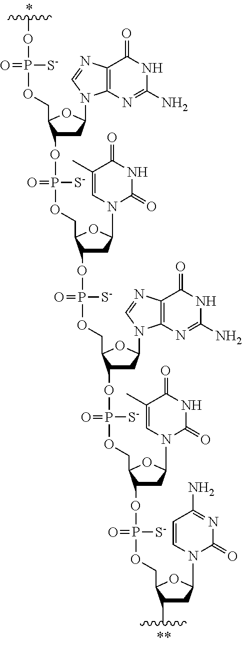 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 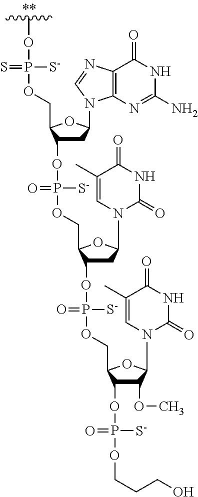 |
| 5.9b | 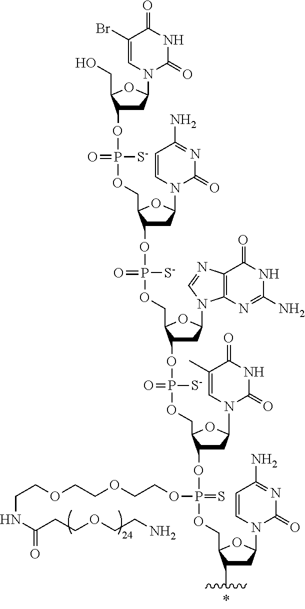 |
| | 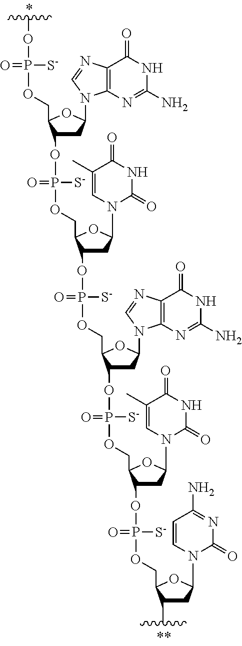 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 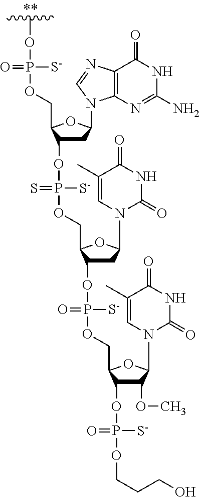 |
| 5.10b | 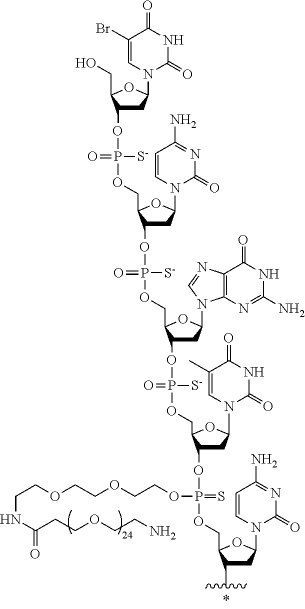 |
| | 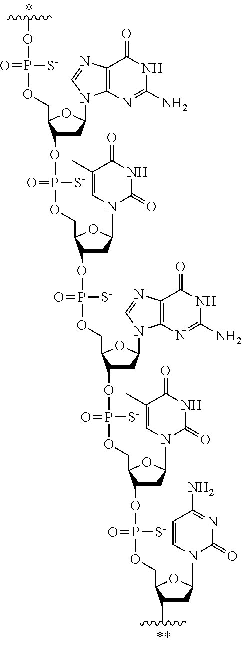 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 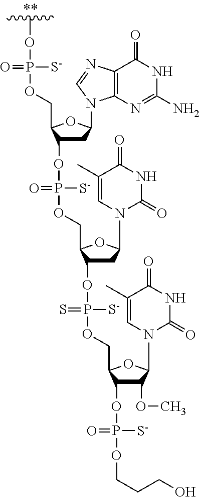 |
| 5.11b | 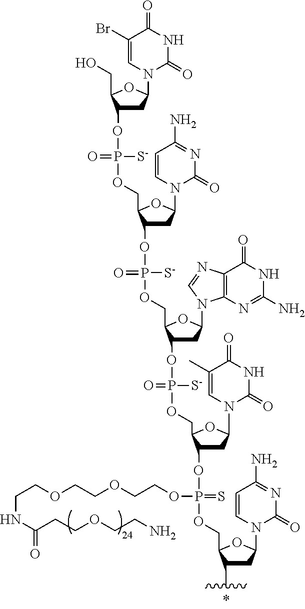 |
| | 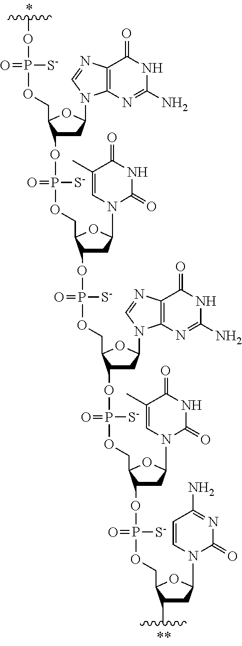 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 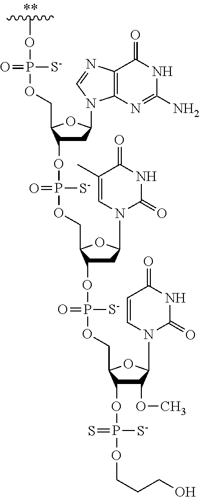 |
| 5.12b | 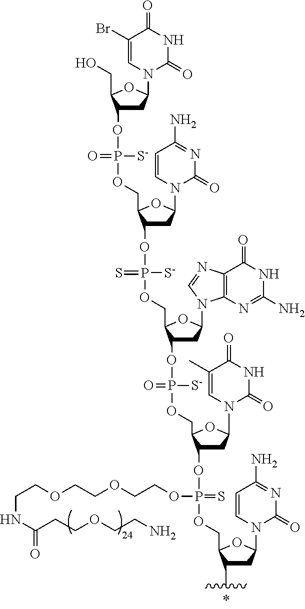 |
| | 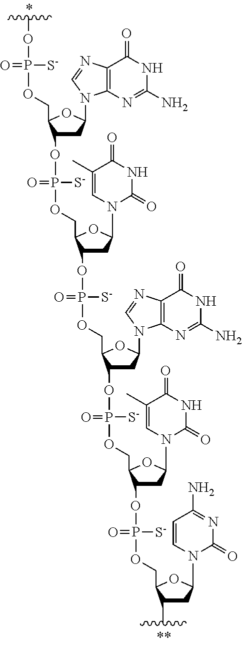 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 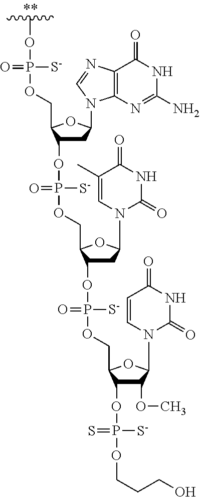 |
| 6.1b | 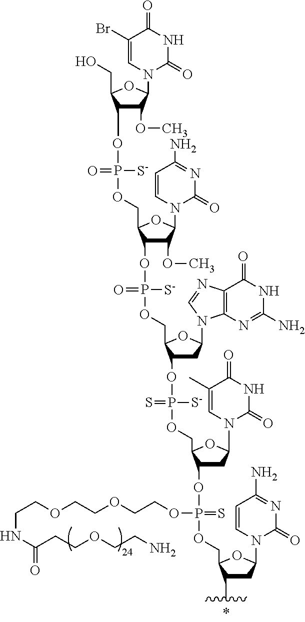 |
| | 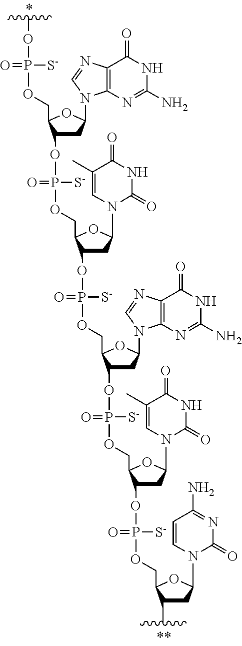 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 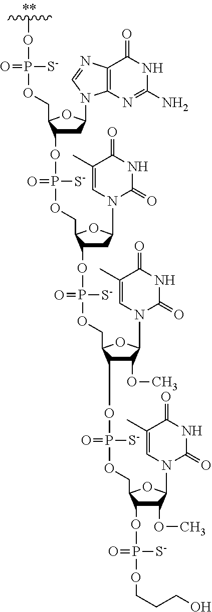 |
| 6.2b | 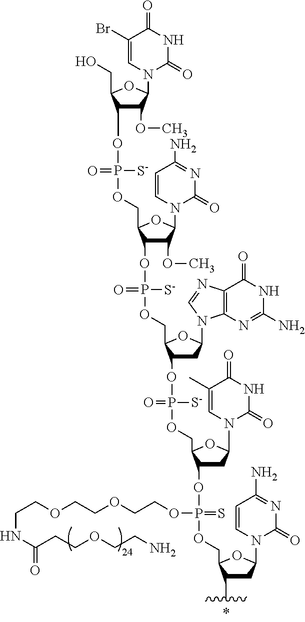 |
| | 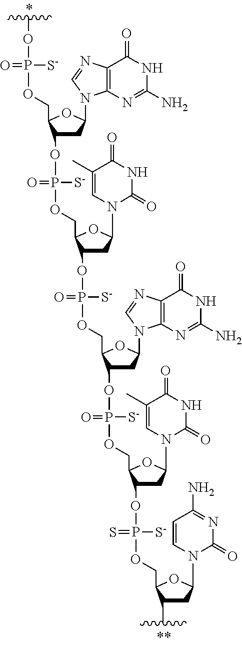 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=S)([S-])OC[C@H]1O[C@@H](n2ccc(N)nc2=O)C[C@H]1*] |
| | 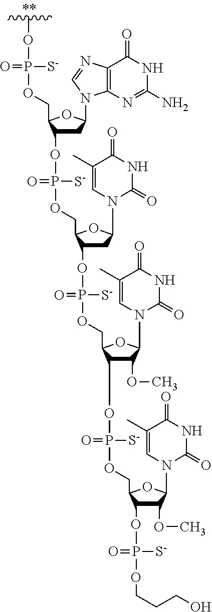 |
| 6.3b | 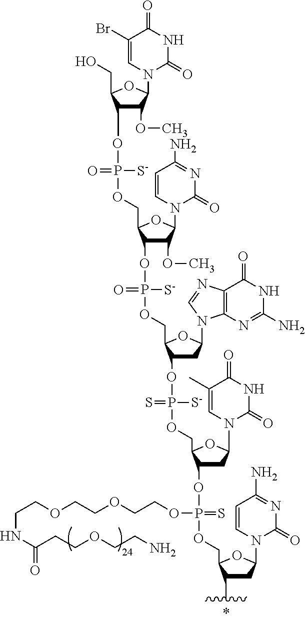 |
| | 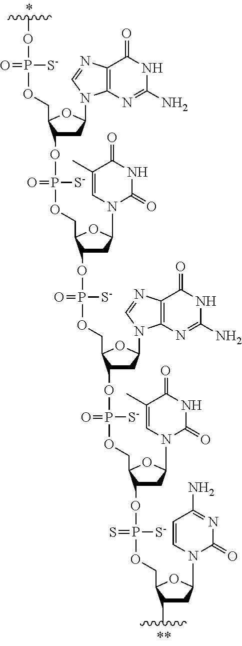 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=S)([S-])OC[C@H]1O[C@@H](n2ccc(N)nc2=O)C[C@H]1*] |
| | 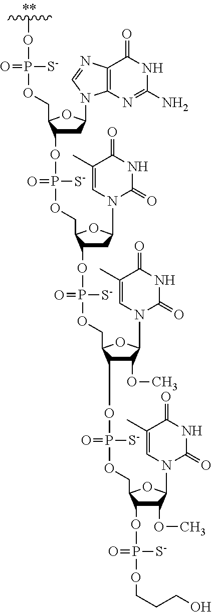 |
| 7.1b | 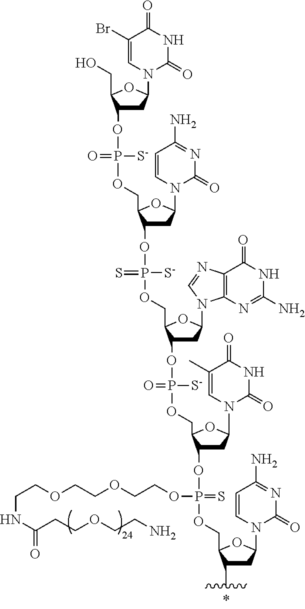 |
| | 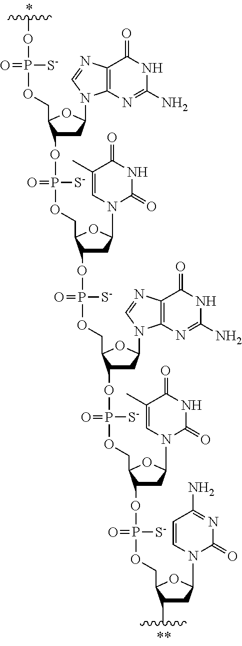 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 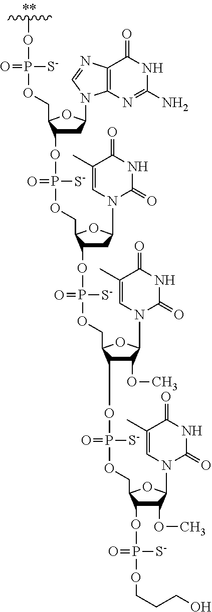 |
| 7.2b | 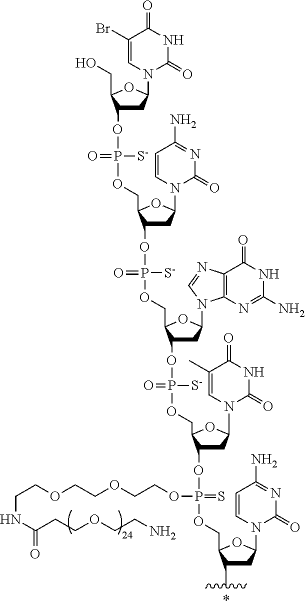 |
| | 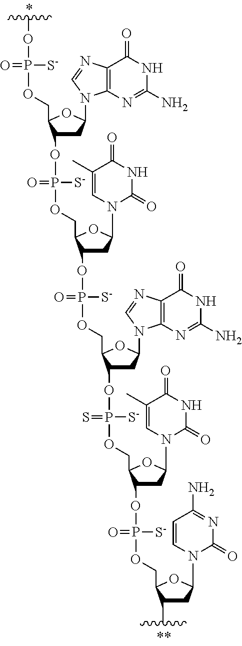 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=S)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 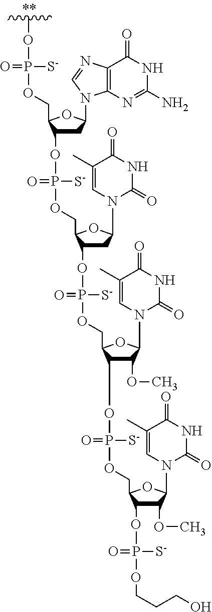 |
| 7.3b | 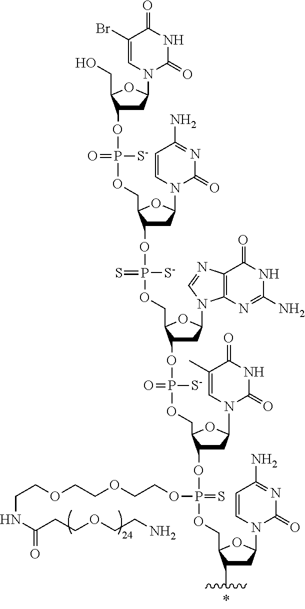 |
| | 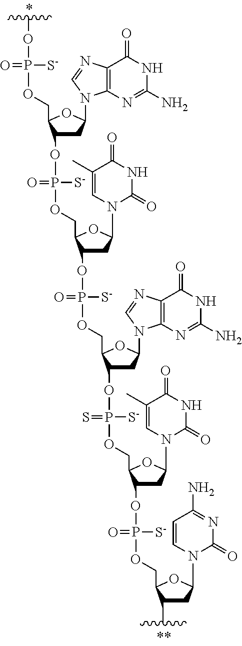 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=S)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 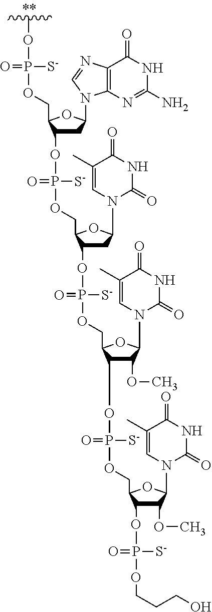 |
| 7.4b | 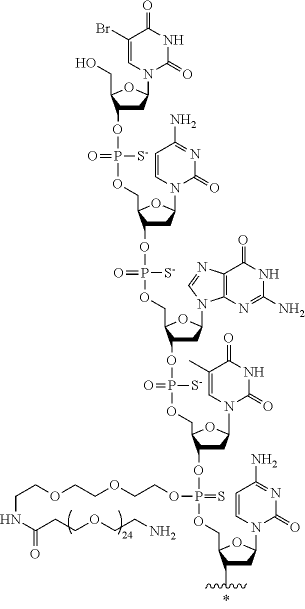 |
| | 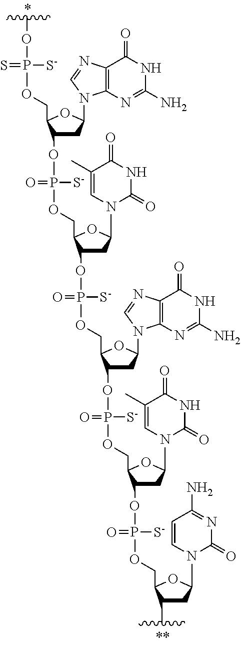 [SMILES: *OP(=S)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 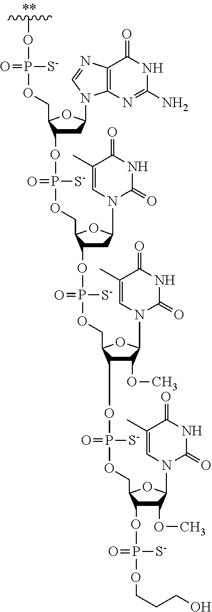 |
| 7.5b | 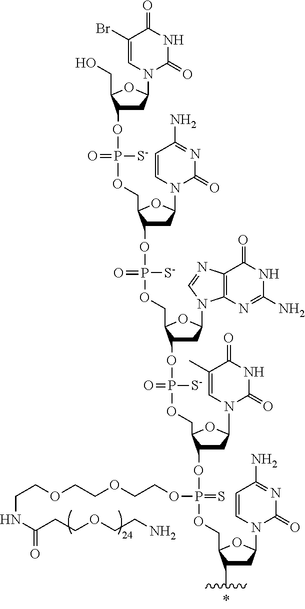 |
| | 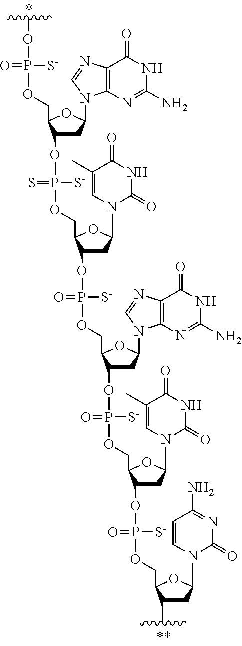 [SMILES: *OP(=O)([S-])OC[C@H]1O[C@@H](n2cnc3c(=O)[nH]c(N)nc32)C[C@H]1OP(=S)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 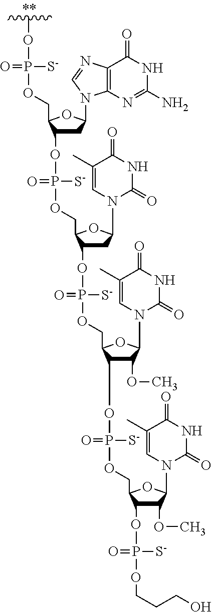 |
| 7.6b | 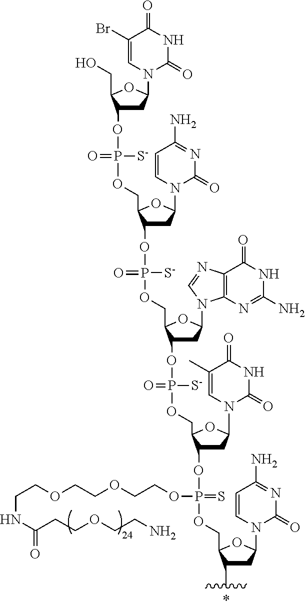 |
| | 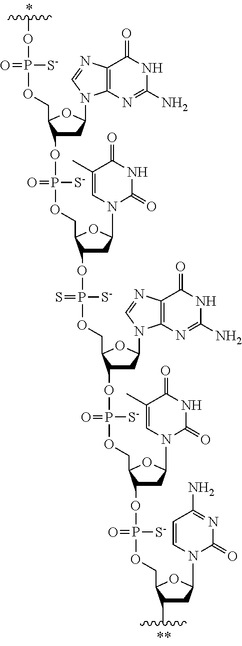 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=S)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 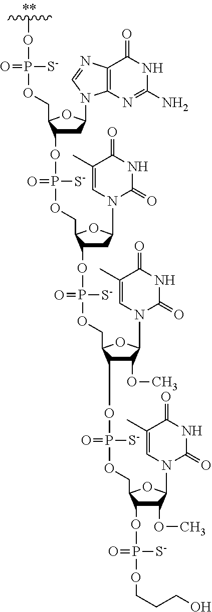 |
| 7.7b | 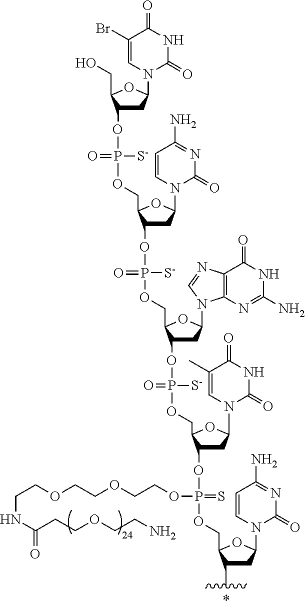 |
| | 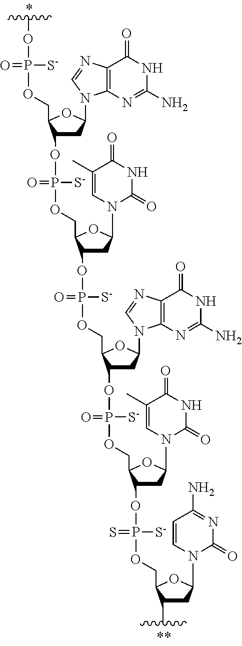 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=S)([S-])OC[C@H]1O[C@@H](n2ccc(N)nc2=O)C[C@H]1*] |
| | 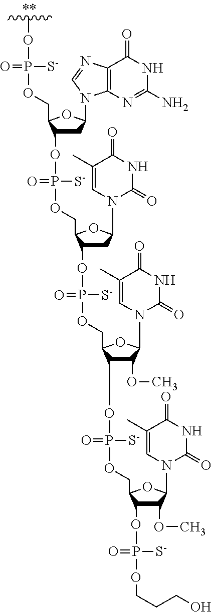 |
| 7.8b | 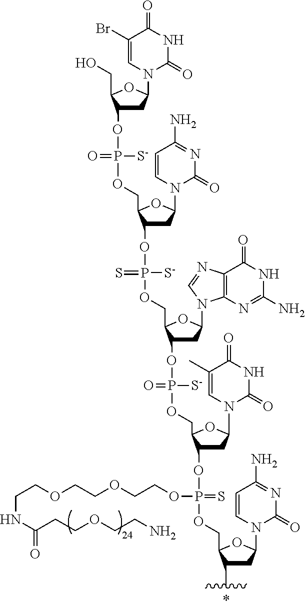 |
| | 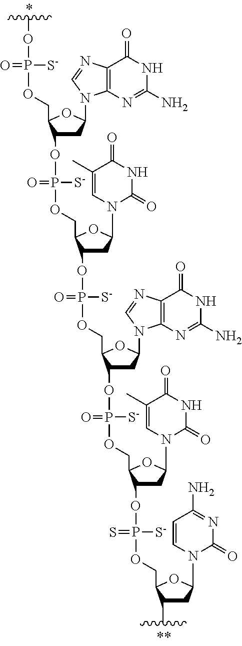 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=S)([S-])OC[C@H]1O[C@@H](n2ccc(N)nc2=O)C[C@H]1*] |
| | 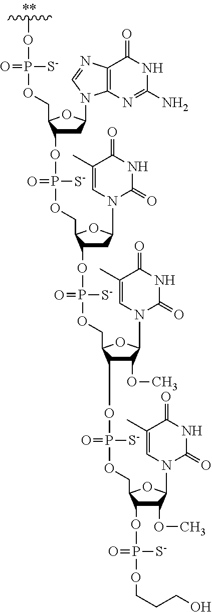 |
| 7.9b | 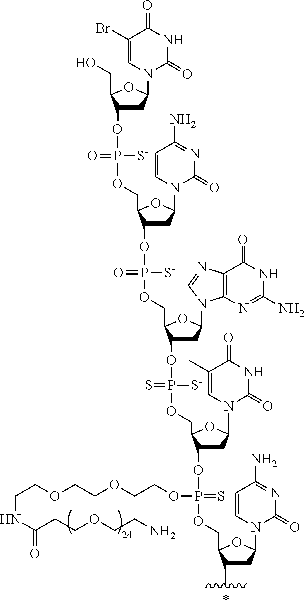 |
| | 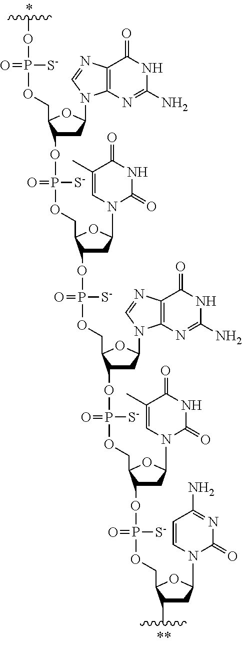 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 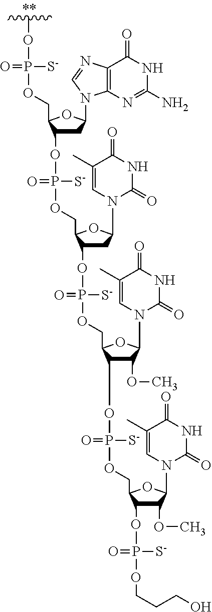 |
| 7.10b | 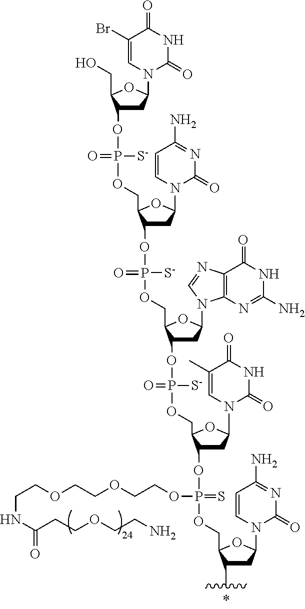 |
| | 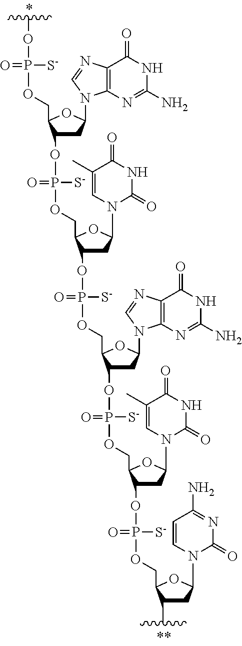 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2ccc(N)nc2=O)C[C@H]1*] |
| | 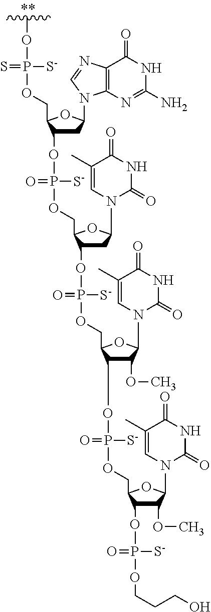 |
| 15.7b | 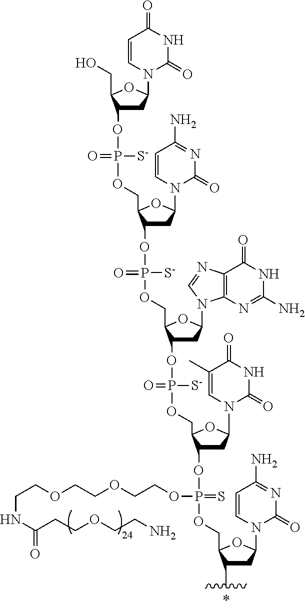 |
| | 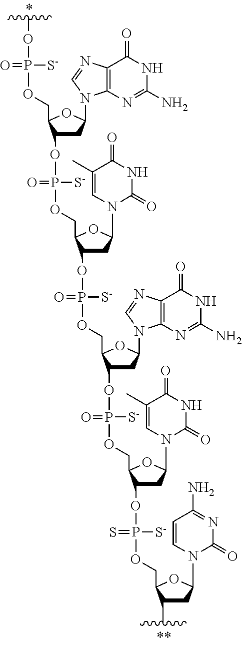 [SMILES: *OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=O)([S-])OC[C@H]1O[C@@H](n2cnc3c(=O)[nH]c(N)nc32)C[C@H]1OP(=O)([S-])OC[C@H]1O[C@@H](n2cc(C)c(=O)[nH]c2=O)C[C@H]1OP(=S)([S-])OC[C@H]1O[C@@H](n2ccc(N)nc2=O)C[C@H]1*] |
| | 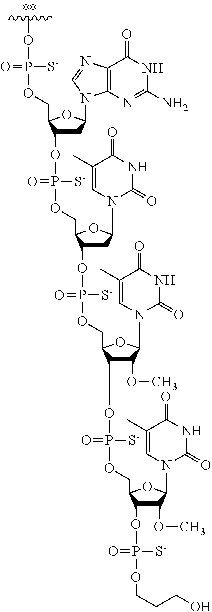 |

As with the oligonucleotides of formula (C), the immunomodulating oligonucleotides of formula (D) may be used as precursors to prepare conjugates comprising a nectin-4 antibody or antigen-binding fragment thereof and one or more immunomodulating oligonucleotides of formula (D) linked via Q-tag as shown in the structures of formula (A) as described herein.

The immunomodulating oligonucleotides of formulae (C) and (D) as described herein may be prepared according to methods known in the art. A general method for the preparation of immunomodulating oligonucleotides, including those provided in the present disclosure, is described in the section titled General Oligonucleotide Synthesis of this disclosure.

### II. B. Linking Moieties

In another aspect, the oligonucleotide is conjugated to the nectin-4 antibody via a linking moiety. The length, rigidity and chemical composition of the linking moiety impact the conjugation reaction rates and the stability of the resulting conjugates. In some embodiments, the linking moiety comprises polyethylene glycol (PEG). In some embodiments, the PEG contains about 10-50 ethylene glycol units. In some embodiments, the linking moiety is an aliphatic chain.

For Formula (A), the linking moiety is represented by L. In some embodiments, the linker L comprises an oligoethylene glycol or polyethylene glycol moiety. In certain embodiments, the linker L is a group comprising the structure

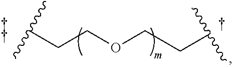

wherein [symbol] † indicates the point of attachment to Y^{PTE}, and [symbol] ‡ indicates the point of attachment to the rest of the conjugate.

In other embodiments, the linker L is a group comprising the structure

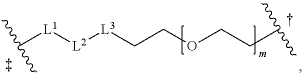

wherein [symbol] † indicates the point of attachment to Y^{PTE}, and [symbol] ‡ indicates the point of attachment to the rest of the conjugate. In some embodiments, L¹ is absent. In some embodiments, L¹ is unsubstituted alkyl. In some embodiments, L¹ is independently an unsubstituted C₁₋₆ alkyl. In some embodiments, each L¹ is methyl or ethyl. In some embodiments, L¹ is independently a substituted alkyl. In some embodiments, L^{T} is independently a substituted C₁₋₆ alkyl. In some embodiments, L¹ is C₁₋₆ alkyl substituted with one or more substituents selected from the group consisting of alkoxy, acyl, acyloxy, alkoxycarbonyl, carbonylalkoxy, acylamino, amino, aminoacyl, aminocarbonylamino, aminocarbonyloxy, cycloalkyl, cycloalkenyl, cyano, azido, halo, hydroxyl, nitro, carboxyl, thiol, thioalkyl, alkyl, alkenyl, alkynyl, heterocyclyl, aminosulfonyl, sulfonylamino, sulfonyl and oxo.

In some embodiments, L² is absent. In some embodiments, L² is unsubstituted or substituted alkyl.

In some embodiments, L³ is absent. In some embodiments, L³ is a linker moiety. In some embodiments, the linker moiety is an unsubstituted or substituted alkyl. In some embodiments, the linker moiety is independently an unsubstituted C₁₋₆ alkyl. In some embodiments, the linker moiety is methyl or ethyl. In some embodiments, the linker moiety is independently a substituted alkyl. In some embodiments, the linker moiety is independently a substituted C₁₋₆ alkyl. In some embodiments, the linker moiety is C₁₋₆ alkyl substituted with one or more substituents selected from the group consisting of alkoxy, acyl, acyloxy, alkoxycarbonyl, carbonylalkoxy, acylamino, amino, aminoacyl, aminocarbonylamino, aminocarbonyloxy, cycloalkyl, cycloalkenyl, cyano, azido, halo, hydroxyl, nitro, carboxyl, thiol, thioalkyl, alkyl, alkenyl, alkynyl, heterocyclyl, aminosulfonyl, sulfonylamino, sulfonyl and oxo. In some embodiments, the linker moiety is an amino acid residue. In some embodiments, the amino acid is selected from the group consisting of glycine, alanine, glutamic acid and proline. In some embodiments, the linker is methyl. In some embodiments, the linker moiety is —R⁵C(O)R⁶NHR⁷—, wherein R⁵, and R⁷ are independently absent or unsubstituted or substituted alkyl and R⁶ is an amino acid residue. In some embodiments, the amino acid is selected from the group consisting of glycine, alanine, glutamic acid and proline. In some embodiments, the linker moiety is —R³C(O)NHR⁴—, wherein R³ and R⁴ are independently absent or unsubstituted or substituted alkyl. In some embodiments, R³ is methylene and R⁴ is —(CH₂)₄—. In some embodiments, R³ is methylene and R⁴ is absent. When more than one oligonucleotide (i.e., p=2), the two L¹ can be different or same, the two L² can be different or same and the two L³ can be different or same.

In some embodiments, m is about 3-10, about 10-15, about 15-20, about 20-25, about 25-30, about 5-16, about 15-30, about 15-25 or about 20-30. In some embodiments, m is 20, 21, 22, 23, 24 or 25. In some embodiments, m is about 24.

In any of the described embodiments, the linker may be a cleavable linker. In any of the described embodiments, the linker may be a non-cleavable linker.

### III. Nectin-4 Antibodies

Described herein, in various embodiments, are antibodies which specifically bind nectin-4 (i.e., anti-nectin-4 antibodies, nectin-4 antibodies, antibodies targeting nectin-4), particularly antibodies which specifically bind human nectin-4, and conjugates thereof. In some embodiments, nectin-4 refers to human nectin-4, and the antibodies specifically bind human nectin-4. Nectin-4 gene and polypeptide sequences (e.g., human gene and polypeptide sequences) are known in the art. In some embodiments, the nectin-4 antibody specifically binds to an extracellular domain of the nectin-4 polypeptide, e.g., a human nectin-4 polypeptide. In some embodiments, the nectin-4 antibody specifically binds to the IgV domain of the nectin-4 polypeptide, e.g., a human nectin-4 polypeptide. In some embodiments, the nectin-4 antibody specifically binds to an extracellular domain which is not an IgV domain of the nectin-4 polypeptide, e.g., a human nectin-4 polypeptide. In some embodiments, the nectin-4 conjugate (i.e., anti-nectin-4 conjugate) specifically binds to a cell (e.g., a tumor cell) that expresses a nectin-4 polypeptide, e.g., a human nectin-4 polypeptide, on its cell surface.

In some embodiments, the oligonucleotide is conjugated to a nectin-4 antibody via one or more Q tags. In some embodiments, the Q tag comprises a glutamine residue which is linked to the rest of the conjugate. In still further embodiments of the present aspect, which may be combined with any of the embodiments herein, each Q tag independently comprises or is a peptide sequence selected from the group consisting of SEQ ID NOs: 39-55. In some embodiments, each Q tag independently comprises or is a peptide sequence selected from the group consisting of the peptide sequences of Table 16. In other embodiments of the present aspect, each Q tag independently comprises or is a peptide sequence selected from the group consisting of SEQ ID NOs: 40-55. In yet other embodiments, each Q tag independently comprises or is a peptide sequence selected from the group consisting of SEQ ID NOs: 47-49. In some embodiments, the Q-tag comprises LLQGG (SEQ ID NO:172), GGGLLQGG (SEQ ID NO:173), RPQGF (SEQ ID NO:47), or RPQGFGPP (SEQ ID NO:49). In some embodiments, the Q-tag comprises a peptide sequence RPQGF (SEQ ID NO:47). In certain embodiments, the Q-tag comprising a peptide sequence RPQGF (SEQ ID NO:47) is selected from the group consisting of RPQGF (SEQ ID NO:47), RPQGFPP (SEQ ID NO:48), and RPQGFGPP (SEQ ID NO:49).

In some embodiments, the nectin-4 antibody is selected from a group consisting of a polyclonal antibody, a monoclonal antibody, a humanized antibody, a human antibody, a chimeric antibody, and an antibody fragment. In some embodiments, the nectin-4 antibody fragment is selected from the group consisting of Fab, Fab′, Fab′-SH, F(ab′)₂, Fv fragments, scFv, single domain antibody, single heavy chain antibody and single light chain antibody. In some embodiments, the nectin-4 antibody is a bispecific antibody (i.e., specifically binds to human nectin-4 and specifically binds to another target than human nectin-4). In some embodiments, the nectin-4 antibody is multispecific (i.e., specifically binds to human nectin-4 and specifically binds to at least two other targets than human nectin-4).

In some embodiments, the nectin-4 antibody or conjugate comprises a VH domain and a VL domain of an antibody designated TNT-201, TNT-202, TNT-203, TNT-204, TNT-205, TNT-206, TNT-207, TNT-208, TNT-209, TNT-210, TNT-211, TNT-212, TNT-317, TNT-318, TNT-320, TNT-321, TNT-322, TNT-323, TNT-324, TNT-327, TNT-328, TNT-330, TNT-331, TNT-332, TNT-333, TNT-334, TNT-335, TNT-336, TNT-337, TNT-412, TNT-413, TNT-414, TNT-417, TNT-418, TNT-419, TNT-420, TNT-421, TNT-422, TNT-423, TNT-424, TNT-425, TNT-426, TNT-427, TNT-428, TNT-429, TNT-430, TNT-431, or TNT-432, wherein the VH domains are as in Table 7 and the VL domains are as in Table 8. In some embodiments, the nectin-4 antibody or conjugate comprises a VH domain comprising a CDR-H1, CDR-H2, and CDR-H3 and a VL domain comprising a CDR-L1, CDR-L2, and CDR-L2 of an antibody designated TNT-201, TNT-202, TNT-203, TNT-204, TNT-205, TNT-206, TNT-207, TNT-208, TNT-209, TNT-210, TNT-211, TNT-212, TNT-317, TNT-318, TNT-320, TNT-321, TNT-322, TNT-323, TNT-324, TNT-327, TNT-328, TNT-330, TNT-331, TNT-332, TNT-333, TNT-334, TNT-335, TNT-336, or TNT-337 wherein CDR-H1, CDR-H2, CDR-H3, CDR-L1, CDR-L2, and CDR-L3 are as in Table 9. In some embodiments, the nectin-4 antibody or conjugate comprises a VH domain comprising a CDR-H1, CDR-H2, and CDR-H3 and a VL domain comprising a CDR-L1, CDR-L2, and CDR-L3 of an antibody designated TNT-201, TNT-202, TNT-203, TNT-204, TNT-205, TNT-206, TNT-207, TNT-208, TNT-209, TNT-210, TNT-211, TNT-212, TNT-317, TNT-318, TNT-320, TNT-321, TNT-322, TNT-323, TNT-324, TNT-327, TNT-328, TNT-330, TNT-331, TNT-332, TNT-333, TNT-334, TNT-335, TNT-336, or TNT-337 wherein CDR-H1, CDR-H2, CDR-H3, CDR-L1, CDR-L2, and CDR-L3 are as in Table 10. In some embodiments, the nectin-4 antibody or conjugate comprises a VH domain comprising a CDR-H1, CDR-H2, and CDR-H3 and a VL domain comprising a CDR-L1, CDR-L2, and CDR-L3 of an antibody designated TNT-201, TNT-202, TNT-203, TNT-204, TNT-205, TNT-206, TNT-207, TNT-208, TNT-209, TNT-210, TNT-211, TNT-212, TNT-317, TNT-318, TNT-320, TNT-321, TNT-322, TNT-323, TNT-324, TNT-327, TNT-328, TNT-330, TNT-331, TNT-332, TNT-333, TNT-334, TNT-335, TNT-336, or TNT-337 wherein CDR-H1, CDR-H2, CDR-H3, CDR-L1, CDR-L2, and CDR-L3 are as in Table 11. In some embodiments, the nectin-4 antibody or conjugate comprises a heavy chain and a light chain of an antibody designated TNT-201, TNT-202, TNT-203, TNT-204, TNT-205, TNT-206, TNT-207, TNT-208, TNT-209, TNT-210, TNT-211, TNT-212, TNT-317, TNT-318, TNT-320, TNT-321, TNT-322, TNT-323, TNT-324, TNT-327, TNT-328, TNT-330, TNT-331, TNT-332, TNT-333, TNT-334, TNT-335, TNT-336, TNT-337, TNT-412, TNT-413, TNT-414, TNT-417, TNT-418, TNT-419, TNT-420, TNT-421, TNT-422, TNT-423, TNT-424, TNT-425, TNT-426, TNT-427, TNT-428, TNT-429, TNT-430, TNT-431, or TNT-432, wherein the light chain is as in Table 14 and (i) the heavy chain comprises a Q tag and is as in Table 15 or (ii) the heavy chain is as in Table 15A.

In some embodiments, the antibody or conjugate comprises a light chain variable domain (VL) and a heavy chain variable domain (VH). In some embodiments, the VH comprises the sequence selected from a group consisting of the sequences disclosed in Table 7, below. In some embodiments, the VL comprises the sequence selected from a group consisting of the sequences disclosed in Table 8, below. In some embodiments, the VL comprises the sequence SEQ ID NO:248 and the VH comprises the sequence SEQ ID NO:249. In some embodiments, the VL comprises the sequence SEQ ID NO:250 and the VH comprises the sequence SEQ ID NO:251. In some embodiments, the VL comprises the sequence SEQ ID NO:252 and the VH comprises the sequence SEQ ID NO:253. In some embodiments, the VL comprises the sequence SEQ ID NO:254 and the VH comprises the sequence SEQ ID NO:255. In some embodiments, the VL comprises the sequence SEQ ID NO:256 and the VH comprises the sequence SEQ ID NO:257. In some embodiments, the VL comprises the sequence SEQ ID NO:258 and the VH comprises the sequence SEQ ID NO:259. In some embodiments, the VL comprises the sequence SEQ ID NO:260 and the VH comprises the sequence SEQ ID NO:261. In some embodiments, the VL comprises the sequence SEQ ID NO:262 and the VH comprises the sequence SEQ ID NO:263. In some embodiments, the VL comprises the sequence SEQ ID NO:264 and the VH comprises the sequence SEQ ID NO:265. In some embodiments, the VL comprises the sequence SEQ ID NO:266 and the VH comprises the sequence SEQ ID NO:267. In some embodiments, the VL comprises the sequence SEQ ID NO:268 and the VH comprises the sequence SEQ ID NO:269. In some embodiments, the VL comprises the sequence SEQ ID NO:270 and the VH comprises the sequence SEQ ID NO:271. In some embodiments, the VL comprises the sequence of SEQ ID NO:900 and the VH comprises the sequence of SEQ ID NO:901. In some embodiments, the VL comprises the sequence of SEQ ID NO:902 and the VH comprises the sequence of SEQ ID NO:903. In some embodiments, the VL comprises the sequence of SEQ ID NO:904 and the VH comprises the sequence of SEQ ID NO:905. In some embodiments, the VL comprises the sequence of SEQ ID NO:906 and the VH comprises the sequence of SEQ ID NO:907. In some embodiments, the VL comprises the sequence of SEQ ID NO:908 and the VH comprises the sequence of SEQ ID NO:909. In some embodiments, the VL comprises the sequence of SEQ ID NO:910 and the VH comprises the sequence of SEQ ID NO:911. In some embodiments, the VL comprises the sequence of SEQ ID NO:912 and the VH comprises the sequence of SEQ ID NO:913. In some embodiments, the VL comprises the sequence of SEQ ID NO:914 and the VH comprises the sequence of SEQ ID NO:915. In some embodiments, the VL comprises the sequence of SEQ ID NO:916 and the VH comprises the sequence of SEQ ID NO:917. In some embodiments, the VL comprises the sequence of SEQ ID NO:918 and the VH comprises the sequence of SEQ ID NO:919. In some embodiments, the VL comprises the sequence of SEQ ID NO:920 and the VH comprises the sequence of SEQ ID NO:921. In some embodiments, the VL comprises the sequence of SEQ ID NO:922 and the VH comprises the sequence of SEQ ID NO:923. In some embodiments, the VL comprises the sequence of SEQ ID NO:924 and the VH comprises the sequence of SEQ ID NO:925. In some embodiments, the VL comprises the sequence of SEQ ID NO:926 and the VH comprises the sequence of SEQ ID NO:927. In some embodiments, the VL comprises the sequence of SEQ ID NO:928 and the VH comprises the sequence of SEQ ID NO:929. In some embodiments, the VL comprises the sequence of SEQ ID NO:930 and the VH comprises the sequence of SEQ ID NO:931.

TABLE 7
Nectin-4 antibody VH domains

| Antibody | Sequence |
|---|---|
| TNT-188; | EVQLVQSGAEVKKPGSSVKVSCKASGYTFTGYYMHWVRQAPGQGLEWMGWIN |
| TNT-201 | PNSGGTNYAQKFQGRVTMTRDTSISTAYMELRSLRSDDTAVYYCARGSALHFFG |
| | VFDYWGQGTLVTVSS (SEQ ID NO: 249) |
| TNT-189; | EVQLVQSGAEVKKPGSSVKVSCKASGGTFSSYAISWVRQAPGQGLEWMGGIIPIF |
| TNT-202 | GTANYAQKFQGRVTITADESTSTAYMELSSLRSEDTAVYYCARGYSSSWYEPYY |
| | FDYWGQGTLVTVSS (SEQ ID NO: 251) |
| TNT-190; | EVQLVQSGGGLVKPGGSLRLSCAASGFTFSDYYMSWVRQAPGKGLEWVSAISGS |
| TNT-203 | GGSTYYADSVKGRFTISRDNSKNTLYLQMNSLRAEDTAVYYCARDEGYYYMDV |
| | WGKGTTVTVSS (SEQ ID NO: 253) |
| TNT-191; | EVQLLESGGGVVQPGRSLRLSCAASGFTFSSYGMHWVRQAPGKGLEWVAVISY |
| TNT-204 | DGSNKYYADSVKGRFTISRDNSKNTLYLQMNSLRAEDTAVYYCAKSSYYDYVW |
| | GSYRYTDAFDIWGQGTMVTVSS (SEQ ID NO: 255) |
| TNT-192; | EVQLLESGGGVVQPGRSLRLSCAASGFTFSSYGMHWVRQAPGKGLEWVAVISY |
| TNT-205 | DGSNKYYADSVKGRFTISRDNSKNTLYLQMNSLRAEDTAVYYCAKDYGYNWN |
| | FFDYWGQGTLVTVSS (SEQ ID NO: 257) |
| TNT-193; | QVQLQESGPGLVKPSGTLSLTCTVSGGSISSGGYYWAWIRQPPGKGLEWIGSIYY |
| TNT-206 | TGSTYYNPSLKSRVTIYVDTSKNQFSLKLSSVTAADTAVYYCARDPFPEPPDAFDI |
| | WGQGTMVTVSS (SEQ ID NO: 259) |
| TNT-194; | EVQLVQSGAEVKKPGASVKVSCKASGYTFTSYDINWVRQAPGQGLEWMGGIIPI |
| TNT-207 | FGTANYAQKFQGRVTITADESTSTAYMELRSLRSEDTAVYYCASTAVAGFGIDY |
| | WGQGTLVTVSS (SEQ ID NO: 261) |
| TNT-195; | EVQLVQSGAEVKKPGSSVKVSCKASGGTFSSYAISWVRQAPGQGLEWMGGIIPIF |
| TNT-208 | GTANYAQKFQGRVTITADKSTSTAYMELSSLRSEDTAVYYCAAGYYYDSSGYY |
| | HQPYYFDYWGQGTLVTVSS (SEQ ID NO: 263) |
| TNT-196; | EVQLVESGGGLVQPGGSLRLSCAASGFTFSDYYMSWIRQAPGKGLEWVSGISWN |
| TNT-209 | SGSIGYADSVKGRFTISRDNSKNTLYLQMNSLRAEDTAVYYCASSGSWYLPLDY |
| | WGQGTLVTVSS (SEQ ID NO: 265) |
| TNT-197; | EVQLVQSGGGVVQPGRSLRLSCAASGFTFSSYAMHWVRQAPGKGLEWVAVISY |
| TNT-210 | DGSNKYYADSVKGRFTISRDNSKNTLYLQMNSLRAEDTAVYYCARATLQLWLP |
| | LFDYWGQGTLVTVSS (SEQ ID NO: 267) |
| TNT-198; | EVQLVQSGAEVKKPGSSVKVSCKASGGTFSSYAISWVRQAPGQGLEWMGWINP |
| TNT-211 | NSGGTNYAQKFQGRVTMTRDTSTSTAYMELSSLRSEDTAVYYCATGYYGDLYY |
| | FDYWGQGTLVTVSS (SEQ ID NO: 269) |
| TNT-153; | EVQLVESGGGLVQPGGSLRLSCAASGFTFSSYNMNWVRQAPGKGLEWVSYISSS |
| TNT-154; | SSTIYYADSVKGRFTISRDNAKNSLSLQMNSLRDEDTAVYYCARAYYYGMDVW |
| TNT-212 | GQGTTVTVSS (SEQ ID NO: 271) |
| TNT-317 | QVQLQESGPGLVKPSETLSLTCTVSGGSISSYFWSWIRQPPGKGLEWIGYIYYSGN |
| | TNYNPSLKSRVTISVDMSKNQFSLRLSSVTAADTAVYYCARDFHHYDGSAYRW |
| | YFDLWGRGTLVTVSS (SEQ ID NO: 901) |
| TNT-318 | QVQLQESGPGLVKPSETLSLTCTVSGGSISSNYWTWIRQSAGKGLEWIGRIHISES |
| | TNYNPSLKSRVTMSVDTSKNQFSLKLSSVTAADTAVYFCAREGEWKVLGAFDIW |
| | GQGTMVTVSS (SEQ ID NO: 903) |
| TNT-320 | QVQLVQSGAEVKKPGASVKVSCKASGYTFTVYYIHWVRQAPGQGLEWMGWISP |
| | NSGGTNYAQKFQGRVTMTRDTSISTAYMELTRLRSDDTAIFYCARQTVVTPNWG |
| | FDYWGQGTLVTVSS (SEQ ID NO: 905) |
| TNT-321 | QVQLQESGPGLVKPSETLSLTCTVSGGSISSYYWSWIRQPPGKGLEWIGYIYYSGN |
| | VNYNPSLKSRVTISVDASKNQFSLKLTSVTAADTAVYYCAIGGVGAQWDAFDIW |
| | GQGTMVTVSS (SEQ ID NO: 907) |
| TNT-322 | QVQLQESGPGLVKPSATLSLTCTVSGGSISSYYWNWIRQPPGKGLEWIGYIYYSG |
| | NTNYNPSLKSRVTISVDMSKNQFSLKLNSVTAADTAVYYCARSESYGYWFFYLW |
| | GRGTLVTVSS (SEQ ID NO: 909) |
| TNT-323 | QVQLQESGPGLVKPSETLSLTCTVSGGSISSYFWNWIRQPPGKGLEWIGYLYYIGT |
| | TNYNPSLKSRVTISVDTSKNQFSLKLNSVTAADTAVYYCARSGNYYNDAFDIWG |
| | QGTMVTVSS (SEQ ID NO: 911) |
| TNT-324 | QVQLVQSGAEVKKPGASVKVSCKASGYTFTGYFIHWVRQAPGQGLEWMGWINP |
| | NSGGTNYAQKFQGRVTMTRDTSISTAYMELSRLRSDDTAVYYCARDTFYYYYG |
| | MDVWGQGTTVTVSS (SEQ ID NO: 913) |
| TNT-327 | QVQLQESGPGLVKPSETLSLTCTVSGGSISDYYWIWIRQPAGKGLEWIGRIYTTGG |
| | AKYNPSLKSRVTMSIDTSKNNFSLKLSSVTAADTAVYFCARDRNWEWNFDLWG |
| | RGTLVTVSS (SEQ ID NO: 915) |
| TNT-328 | QVQLQESGPGLVKPSETLSLTCTVSGGSMSSYFWSWIRQPPERGLEWVGYMYYS |
| | GNTNYNPSLKSRVTTSIDTSKNQFSLKLTSVTAADTAVYYCARSGNFYWNFDLW |
| | GRGTLVTVSS (SEQ ID NO: 917) |
| TNT-330 | EVQLVESGGGLVKPGGSLRLSCAVSGFTFSSYNMNWVRQAPGKGLEWVSSISISS |
| | SYIYYADSVKGRFTISRDNAKNSLYLQMNSLRAEDTAVYYCARDRGLDDAFDIW |
| | GQGTVVTVSS (SEQ ID NO: 919) |
| TNT-331 | QVHLQESGPGLVKPSETLSLTCTVSGGSIRSYYWSWIRQPAGKGLEWIGRIYTSGS |
| | TNYNPSLKSRVTMSVDTSKNRLSLKLSSVTAADTAVYYCAKENWGPSFDYWGQ |
| | GTLVTVSS (SEQ ID NO: 921) |
| TNT-332 | QVQLVQSGAEAKKPGASVRVSCKTSGYTFTNYYIHWVRQAPGQGLEWMGIINPT |
| | LDSTSYAQKFQDRVTMTRDTSTNTDYMDLSSLRFEDTAVYYCARSAGFDAFDIW |
| | GQGTMVTVSS (SEQ ID NO: 923) |
| TNT-333 | QVQLQESGPGLVKPSETLSLTCTISGGSISSYYWSWIRQPPGKGLEWIGYIFYIGNT |
| | NYYPSLKSRVTISVDASKNQFSLKLSSVTAADTAVYYCARSYPYWYFDLWGRGT |
| | LVTVSS (SEQ ID NO: 925) |
| TNT-334 | EVQLVESGGGLVQPGGSLRLSCAASGFTFSRYWMHWVRQAPGKGLVWVSRINS |
| | DGSSSNYADSVKGRFTFSRDNAKNTLYLQMNSLRAEDTAVYFCVRDNWYFDY |
| | WGQGTLVTVSS (SEQ ID NO: 927) |
| TNT-335 | QVQLQESGPGLVKPSETLSLTCTVSGGSISSYYWSWIRQPPGKGLEWIGYIHYSGI |
| | SNYNPSLKSRVAISLDTSNNHFSLKLTSLTAADTAVYFCARGSYWFDPWGQGTL |
| | VTVSS (SEQ ID NO: 929) |
| TNT-336 | KVQLVESGGGLVQPGGSLRLSCEASGFTFSNYWMHWVRQVPGEGLVWVSRLN |
| | NGGTSTRYADSVKSRFTISRDNAKNTLYLQMNSLRAEDTAVYYCVRDWYFDLW |
| | GRGTLVTVSS (SEQ ID NO: 931) |
| TNT-337 | EVQLVESGGGLVQPGGSLRLSCAASGFTFSNYDMSWVRQAPGKGLEWVATISSG |
| | GSYTYYVDSVKGRFTISRDNAKNSLYLQMNSLRAEDTAVYYCARQELGSYYAM |
| | DYWGQGTTVTVSS (SEQ ID NO: 933) |
| TNT-412 | QVQLVQSGAEVKKPGASVKVSCKASGYTFTGYYMHWVRQAPGQGLEWMGWI |
| | NPNSGGTNYAQKFQGRVTMTRDTSISTAYMELSRLRSDDTAVYYCARGSALHFF |
| | GVFDYWGQGTLVTVSS (SEQ ID NO: 935) |
| TNT-417 | QVQLVQSGAEVKKPGSSVKVSCKASGGTFSSYAISWVRQAPGQGLEWMGGIIPIF |
| | GTANYAQKFQGRVTITADKSTSTAYMELSSLRSEDTAVYYCAAGYYYDSSGYY |
| | HQPYYFDYWGQGTLVTVSS (SEQ ID NO: 937) |
| TNT-418 | QVQLQESGPGLVKPSETLSLTCTVSGGSISDYYWIWIRQPAGKGLEWIGRIYTTGG |
| | AKYNPSLKSRVTMSVDTSKNQFSLKLSSVTAADTAVYFCARDRNWEWNFDLWG |
| | RGTLVTVSS (SEQ ID NO: 939) |
| TNT-419 | QVQLQESGPGLVKPSETLSLTCTVSGGSISDYYWSWIRQPAGKGLEWIGRIYTTG |
| | GAKYNPSLKSRVTMSVDTSKNQFSLKLSSVTAADTAVYYCARDRNWEWNFDL |
| | WGRGTLVTVSS (SEQ ID NO: 941) |
| TNT-420 | QVQLQESGPGLVKPSETLSLTCTVSGGSMSSYFWSWIRQPPGKGLEWIGYMYYS |
| | GNTNYNPSLKSRVTISVDTSKNQFSLKLSSVTAADTAVYYCARSGNFYWNFDLW |
| | GRGTLVTVSS (SEQ ID NO: 943) |
| TNT-421 | QVQLQESGPGLVKPSETLSLTCTVSGGSISSYYWSWIRQPPGKGLEWIGYIFYIGN |
| | TNYYPSLKSRVTISVDTSKNQFSLKLSSVTAADTAVYYCARSYPYWYFDLWGRG |
| | TLVTVSS (SEQ ID NO: 945) |
| TNT-413 | EVQLVQSGAEVKKPGSSVKVSCKASGYTFTGYYMHWVRQAPGQGLEWMGWIN |
| | PNSGGTNYAQKFQGRVTMTRDTSISTAYMELRSLRSDDTAVYYCARGSALHFFG |
| | VFDYWGQGTLVTVSS (SEQ ID NO: 947) |
| TNT-422 | EVQLVQSGAEVKKPGASVKVSCKASGYTFTSYDINWVRQAPGQGLEWMGGIIPI |
| | FGTANYAQKFQGRVTITADESTSTAYMELRSLRSEDTAVYYCASTAVAGFGIDY |
| | WGQGTLVTVSS (SEQ ID NO: 949) |
| TNT-423 | EVQLVQSGAEVKKPGSSVKVSCKASGGTFSSYAISWVRQAPGQGLEWMGGIIPIF |
| | GTANYAQKFQGRVTITADKSTSTAYMELSSLRSEDTAVYYCAAGYYYDSSGYY |
| | HQPYYFDYWGQGTLVTVSS (SEQ ID NO: 951) |
| TNT-424 | EVQLVQSGGGVVQPGRSLRLSCAASGFTFSSYAMHWVRQAPGKGLEWVAVISY |
| | DGSNKYYADSVKGRFTISRDNSKNTLYLQMNSLRAEDTAVYYCARATLQLWLP |
| | LFDYWGQGTLVTVSS (SEQ ID NO: 953) |
| TNT-425 | QVQLQESGPGLVKPSETLSLTCTVSGGSISDYYWIWIRQPAGKGLEWIGRIYTTGG |
| | AKYNPSLKSRVTMSIDTSKNNFSLKLSSVTAADTAVYFCARDRNWEWNFDLWG |
| | RGTLVTVSS (SEQ ID NO: 955) |
| TNT-426 | QVQLQESGPGLVKPSETLSLTCTVSGGSMSSYFWSWIRQPPERGLEWVGYMYYS |
| | GNTNYNPSLKSRVTTSIDTSKNQFSLKLTSVTAADTAVYYCARSGNFYWNFDLW |
| | GRGTLVTVSS (SEQ ID NO: 957) |
| TNT-427 | QVQLQESGPGLVKPSETLSLTCTISGGSISSYYWSWIRQPPGKGLEWIGYIFYIGNT |
| | NYYPSLKSRVTISVDASKNQFSLKLSSVTAADTAVYYCARSYPYWYFDLWGRGT |
| | LVTVSS (SEQ ID NO: 959) |
| TNT-414 | QVQLVQSGAEVKKPGASVKVSCKASGYTFTGYYMHWVRQAPGQGLEWMGWI |
| | NPNSGGTNYAQKFQGRVTMTRDTSISTAYMELSRLRSDDTAVYYCARGSALHFF |
| | GVFDYWGQGTLVTVSS (SEQ ID NO: 961) |
| TNT-428 | QVQLVQSGAEVKKPGSSVKVSCKASGGTFSSYAISWVRQAPGQGLEWMGGIIPIF |
| | GTANYAQKFQGRVTITADKSTSTAYMELSSLRSEDTAVYYCAAGYYYDSSGYY |
| | HQPYYFDYWGQGTLVTVSS (SEQ ID NO: 963) |
| TNT-429 | QVQLQESGPGLVKPSETLSLTCTVSGGSISDYYWIWIRQPAGKGLEWIGRIYTTGG |
| | AKYNPSLKSRVTMSVDTSKNQFSLKLSSVTAADTAVYFCARDRNWEWNFDLWG |
| | RGTLVTVSS (SEQ ID NO: 965) |
| TNT-430 | QVQLQESGPGLVKPSETLSLTCTVSGGSISDYYWSWIRQPAGKGLEWIGRIYTTG |
| | GAKYNPSLKSRVTMSVDTSKNQFSLKLSSVTAADTAVYYCARDRNWEWNFDL |
| | WGRGTLVTVSS (SEQ ID NO: 967) |
| TNT-431 | QVQLQESGPGLVKPSETLSLTCTVSGGSMSSYFWSWIRQPPGKGLEWIGYMYYS |
| | GNTNYNPSLKSRVTISVDTSKNQFSLKLSSVTAADTAVYYCARSGNFYWNFDLW |
| | GRGTLVTVSS (SEQ ID NO: 969) |
| TNT-432 | QVQLQESGPGLVKPSETLSLTCTVSGGSISSYYWSWIRQPPGKGLEWIGYIFYIGN |
| | TNYYPSLKSRVTISVDTSKNQFSLKLSSVTAADTAVYYCARSYPYWYFDLWGRG |
| | TLVTVSS (SEQ ID NO: 971) |

TABLE 8
Nectin-4 antibody VL domains

| Antibody | Sequence |
|---|---|
| TNT-188; | DIVMTQSPSSLSASVGDRVTITCRASQSISSYLNWYQQKPGKAPKLLIYAASSLQS |
| TNT-201 | GVPSRFSGSGSGTDFTLTISSLQPEDFATYYCQQSYSTPLTFGQGTKVEIK (SEQ ID |
| | NO: 248) |
| TNT-189; | DIQLTQSPSSLSASVGDRVTITCRASQGISNYLAWYQQKPGKAPKLLIYAASTLQS |
| TNT-202 | GVPSRFSGSGSGTDFTLTISSLQPEDFATYYCQQSYSTPYTFGQGTKLEIK (SEQ ID |
| | NO: 250) |
| TNT-190; | EIVLTQSPATLSLSPGERATLSCRASQSVSSYLAWYQQKPGQAPRLLIYDASNRAT |
| TNT-203 | GIPARFSGSGSGTDFTLTISSLQAEDFATYYCQQSYSTRFGGGTKVEIK (SEQ ID |
| | NO: 252) |
| TNT-191; | EIVLTQSPATLSLSPGERATLSCRASQSVSSSYLAWYQQKPGQAPRLLIYGASSRA |
| TNT-204 | TGIPDRFSGSGSGTDFTLTISRLEPEDFAVYYCQQYNNWWTFGQGTKVDIK (SEQ |
| | ID NO: 254) |
| TNT-192; | DIVMTQSPSSLSASVGDRVTITCRASQGISNYLAWYQQKPGKVPKLLIYAASTLQ |
| TNT-205 | SGVPSRFSGSGSGTDFTLTISSLQPEDVATYYCQKYNSAPYTFGQGTKVEIK (SEQ |
| | ID NO: 256) |
| TNT-193; | EIVLTQSPATLSVSPGERATLSCRASQSVSSNLAWYQQKPGQAPRLLIYGASSRAT |
| TNT-206 | GIPDRFSGSGSGTDFTLTISRLEPEDFAVYYCHQYGSSPLTFGGGTKLEIK (SEQ ID |
| | NO: 258) |
| TNT-194; | DIQMTQSPSSLSASVGDRVTITCRASQGISNYLAWFQQKPGKAPKSLISAASSLQS |
| TNT-207 | GVPSRFSGSGSGTDFTLTISSLQPEDFATYYCLQHDSFPITFGGGTKLEIK (SEQ ID |
| | NO: 260) |
| TNT-195; | DIQMTQSPSTLSASVGDRVTITCRASQGISNYLAWYQQKPGKAPKLLIYAASSLQ |
| TNT-208 | SGVPSRFSGSGSGTDFTLTISSLQPEDFATYYCQQSYSTPLTFGGGTKVEIK (SEQ |
| | ID NO: 262) |
| TNT-196; | DIQMTQSPSSLSASVGDRVTITCQASQDISNYLNWYQQKPGKVPKLLIYAASTLQ |
| TNT-209 | SGVPSRFSGSGSGTDFTLTISSLQPEDFATYYCQQSYSTPLTFGGGTKVEIK (SEQ |
| | ID NO: 264) |
| TNT-197; | EIVLTQSPGTLSLSPGERATLSCRASQSVSSSYLAWYQQKPGQAPRLLIYGASSRA |
| TNT-210 | TGIPDRFSGSGSGTDFTLTISRLEPEDFAVYYCQQYGSSLTFGGGTKVEIK (SEQ ID |
| | NO: 266) |
| TNT-198; | DIVMTQSPSSLSASVGDRVTITCRASQGISNYLAWYQQKPGKVPKLLIYAASTLQ |
| TNT-211 | SGVPSRFSGSGSGTEFTLAISSLQPEDLATYYCQQLNSHPITFGQGTRLEIK (SEQ |
| | ID NO: 268) |
| TNT-153; | DIQMTQSPSSVSASVGDRVTITCRASQGISGWLAWYQQKPGKAPKFLIYAASTLQ |
| TNT-154; | SGVPSRFSGSGSGTDFTLTISSLQPEDFATYYCQQANSFPPTFGGGTKVEIK (SEQ |
| TNT-212 | ID NO: 270) |
| TNT-317 | QSVLTQPPSVSAAPGQKVTISCSGSSSNIGNNYVSWYQQFPGTAPKLLIYDNNKRP |
| | SGIPDRFSGSKSGTSATLGITGLQTGDEADYYCGTWDSSLNAGWVFGGGTKLTV |
| | L (SEQ ID NO: 900) |
| TNT-318 | QTVLTQPSSLSASPGASASLTCTLRSGINVGSYRIYWYQQKPGSPPQFLLRYKSDS |
| | DKQQGSGVPSRFSGSKDDSANAGILLISGLQSEDEADYYCMIWHSSASVFGTGTK |
| | VTVL (SEQ ID NO: 902) |
| TNT-320 | QSVLTQSPSASGTPGQRVTISCSGSSSNIGSNTVHWYQQLPGTAPKLLMYGNYQR |
| | PSGVPARFSGSKSGTSASLAISGLQSEDEADYYCATWDDSLNGWVFGGGTKVTV |
| | L (SEQ ID NO: 904) |
| TNT-321 | DIQMTQSPSAMSASVGDRVTITCRASQGISNYLAWFQQKPGKVPKRLIYAASSLQ |
| | SGVPSRFSGSGSGTEFTLTISSLQPEDFATYYCLQHSFYPLTFGPGTKVDIK (SEQ |
| | ID NO: 906) |
| TNT-322 | QSVLTQPPSVSAAPGQKVTISCSGSSSNIGNNYVSWYQQFPGTAPKLLIYDNNKRP |
| | SGIPDRFSGSKSGTSATLGITGLQTGDEADYYCGTWDSSLSAGVVFGGGTKLTVL |
| | (SEQ ID NO: 908) |
| TNT-323 | DVVMTQSPLSLPVTLGQPASMSCRSSQSLAYSDGNTYLNWFQQRPGQSPRRLIY |
| | KVSNRDSGVPDRFSGSGSGTDFTLKISRVEAEDVGVYYCMQGTHWPLTFGGGTK |
| | VEIK (SEQ ID NO: 910) |
| TNT-324 | QTVVTQEPSLTVSPGGTVTLTCASSTGAVTSGYYPNWFQQKPGQAPRALIYSTSN |
| | KHSWTPARFSGSLLGGKAALTLSGVQPEDEAEYYCLLYYDGAHLFGGGTKLTVL |
| | (SEQ ID NO: 912) |
| TNT-327 | QSALTQPASVSGSPGQSITISCTGTTSDIGSYNLVSWYQQHPGKAPKLMIYEVSMS |
| | PSGVSDRFSGSKSGSTASLTISGLQAEDEADYYCSSYAGSSTVVFGGGTRLTVL |
| | (SEQ ID NO: 914) |
| TNT-328 | QSVLTQPPSVSGAPGQRVTISCTGSSSNIGAGYNVHWYQQLPGTAPKVLISGNSN |
| | RPSGVPDRFSGSKSGTSASLAITGLQAEDEADYYCQSYDSSLSGSAVFGGGTKLT |
| | VL (SEQ ID NO: 916) |
| TNT-330 | DIVMTQTPLSLPVTPGEPASISCRSSQSLLDSDDGNTYLDWYLQKPGQSPQLLIYT |
| | LSYRASGVPDRFSGSGSGTDFTLKVSRVEAEDVGVYYCMQRIEFPYTFGQGTKLE |
| | IK (SEQ ID NO: 918) |
| TNT-331 | QSVLTQPPSVSAAPGQKVTISCSGSSSNIGNNYVSWYQQLPGTAPKLLIYDNNKR |
| | PSGIPDRFSGSKSGTSATLGITGLQTGDEADYYCGTWDTSLSSGWLFGGGTKLTV |
| | L (SEQ ID NO: 920) |
| TNT-332 | DIQLTQSPSFLSASVGDKVTITCRASQGINSYLAWYQQKPGKAPKLLIYSASTLQG |
| | GVPSRFSGSGSGTEFTLTISNLQPADFATYCCQQLNSYPFTFGPGTKVDIK (SEQ ID |
| | NO: 922) |
| TNT-333 | QSVLTQPPSVSAAPGQKVTISCSGSSSNIGNNYVSWYQQLPGTAPKLLISDNNKRP |
| | SGIPDRFSGSKSGTSATLGITGLQTGDEADYYCGTWDSSLSAGVVFGGGTKLTVL |
| | (SEQ ID NO: 924) |
| TNT-334 | EIVMTQSPATLPVSPGERATLSCRASQSVSINLAWYQQKPGQAPRLLIYGVSTRAT |
| | GIPARFSGSGSGTEFTLTISGLQSEDFAVYYCQQYNNWPRTFGQGTKVEIK (SEQ |
| | ID NO: 926) |
| TNT-335 | QSVLTQPPSASGTPGQRVTISCSGSSSNIGSDYIYWYQQLPGTAPKLLIYSNHQRPS |
| | GVPDRFSGSKSGTSASLAISGLRSEDEADYYCAAWEDSLSGPIFGGGTKLTVL |
| | (SEQ ID NO: 928) |
| TNT-336 | QLVLTQSPSASASLGASVKLTCTLSSGHSFYAIAWHQQQPEKGPRYLMQVNSNG |
| | SHNKGDGIPDRFSGSSSGAERYLTISSLQSEDEADYYCQSWDTDIWVFGGGTKLT |
| | VL (SEQ ID NO: 930) |
| TNT-337 | DIVMTQTPLSLPVTPGEPASISCRSSQSIVHSNANTYLEWYLQKPGQSPQLLIYKVS |
| | NRFSGVPDRFSGSGSGTDFTLKISRVEAEDVGVYYCFQGSHVPYTFGGGTKVEIK |
| | (SEQ ID NO: 932) |
| TNT-412 | DIQMTQSPSSLSASVGDRVTITCRASQSISSYLNWYQQKPGKAPKLLIYAASSLQS |
| | GVPSRFSGSGSGTDFTLTISSLQPEDFATYYCQQSYSTPLTFGQGTKVEIK (SEQ ID |
| | NO: 934) |
| TNT-417 | DIQMTQSPSSLSASVGDRVTITCRASQGISNYLAWYQQKPGKAPKLLIYAASSLQS |
| | GVPSRFSGSGSGTDFTLTISSLQPEDFATYYCQQSYSTPLTFGGGTKVEIK (SEQ ID |
| | NO: 936) |
| TNT-418 | QSALTQPASVSGSPGQSITISCTGTTSDIGSYNLVSWYQQHPGKAPKLMIYEVSMS |
| | PSGVSNRFSGSKSGNTASLTISGLQAEDEADYYCSSYAGSSTVVFGGGTRLTVL |
| | (SEQ ID NO: 938) |
| TNT-419 | QSALTQPASVSGSPGQSITISCTGTTSDIGSYNLVSWYQQHPGKAPKLMIYEVSMS |
| | PSGVSNRFSGSKSGNTASLTISGLQAEDEADYYCSSYAGSSTVVFGGGTRLTVL |
| | (SEQ ID NO: 940) |
| TNT-420 | QSVLTQPPSVSGAPGQRVTISCTGSSSNIGAGYNVHWYQQLPGTAPKLLIYGNSN |
| | RPSGVPDRFSGSKSGTSASLAITGLQAEDEADYYCQSYDSSLSGSAVFGGGTKLT |
| | VL (SEQ ID NO: 942) |
| TNT-421 | QSVLTQPPSVSAAPGQKVTISCSGSSSNIGNNYVSWYQQLPGTAPKLLIYDNNKR |
| | PSGIPDRFSGSKSGTSATLGITGLQTGDEADYYCGTWDSSLSAGVVFGGGTKLTV |
| | L (SEQ ID NO: 944) |
| TNT-413 | DIVMTQSPSSLSASVGDRVTITCRASQSISSYLNWYQQKPGKAPKLLIYAASSLQS |
| | GVPSRFSGSGSGTDFTLTISSLQPEDFATYYCQQSYSTPLTFGQGTKVEIK (SEQ ID |
| | NO: 946) |
| TNT-422 | DIQMTQSPSSLSASVGDRVTITCRASQGISNYLAWFQQKPGKAPKSLISAASSLQS |
| | GVPSRFSGSGSGTDFTLTISSLQPEDFATYYCLQHDSFPITFGGGTKLEIK (SEQ ID |
| | NO: 948) |
| TNT-423 | DIQMTQSPSTLSASVGDRVTITCRASQGISNYLAWYQQKPGKAPKLLIYAASSLQ |
| | SGVPSRFSGSGSGTDFTLTISSLQPEDFATYYCQQSYSTPLTFGGGTKVEIK (SEQ |
| | ID NO: 950) |
| TNT-424 | EIVLTQSPGTLSLSPGERATLSCRASQSVSSSYLAWYQQKPGQAPRLLIYGASSRA |
| | TGIPDRFSGSGSGTDFTLTISRLEPEDFAVYYCQQYGSSLTFGGGTKVEIK (SEQ ID |
| | NO: 952) |
| TNT-425 | QSALTQPASVSGSPGQSITISCTGTTSDIGSYNLVSWYQQHPGKAPKLMIYEVSMC |
| | PSGVSDRFSGSKSGSTASLTISGLQAEDEADYYCCSYAGSSTVVFGGGTRLTVL |
| | (SEQ ID NO: 954) |
| TNT-426 | QSVLTQPPSVSGAPGQRVTISCTGSSSNIGAGYNVHWYQQLPGTAPKVLISGNSN |
| | RPSGVPDRFSGSKSGTSASLAITGLQAEDEADYYCQSYDSSLSGSAVFGGGTKLT |
| | VL (SEQ ID NO: 956) |
| TNT-427 | QSVLTQPPSVSAAPGQKVTISCSGSSSNIGNNYVSWYQQLPGTAPKLLISDNNKRP |
| | SGIPDRFSGSKSGTSATLGITGLQTGDEADYYCGTWDSSLSAGVVFGGGTKLTVL |
| | (SEQ ID NO: 958) |
| TNT-414 | DIQMTQSPSSLSASVGDRVTITCRASQSISSYLNWYQQKPGKAPKLLIYAASSLQS |
| | GVPSRFSGSGSGTDFTLTISSLQPEDFATYYCQQSYSTPLTFGQGTKVEIK (SEQ ID |
| | NO: 960) |
| TNT-428 | DIQMTQSPSSLSASVGDRVTITCRASQGISNYLAWYQQKPGKAPKLLIYAASSLQS |
| | GVPSRFSGSGSGTDFTLTISSLQPEDFATYYCQQSYSTPLTFGGGTKVEIK (SEQ ID |
| | NO: 962) |
| TNT-429 | QSALTQPASVSGSPGQSITISCTGTTSDIGSYNLVSWYQQHPGKAPKLMIYEVSMS |
| | PSGVSNRFSGSKSGNTASLTISGLQAEDEADYYCSSYAGSSTVVFGGGTRLTVL |
| | (SEQ ID NO: 964) |
| TNT-430 | QSALTQPASVSGSPGQSITISCTGTTSDIGSYNLVSWYQQHPGKAPKLMIYEVSMS |
| | PSGVSNRFSGSKSGNTASLTISGLQAEDEADYYCSSYAGSSTVVFGGGTRLTVL |
| | (SEQ ID NO: 966) |
| TNT-431 | QSVLTQPPSVSGAPGQRVTISCTGSSSNIGAGYNVHWYQQLPGTAPKLLIYGNSN |
| | RPSGVPDRFSGSKSGTSASLAITGLQAEDEADYYCQSYDSSLSGSAVFGGGTKLT |
| | VL (SEQ ID NO: 968) |
| TNT-432 | QSVLTQPPSVSAAPGQKVTISCSGSSSNIGNNYVSWYQQLPGTAPKLLIYDNNKR |
| | PSGIPDRESGSKSGTSATLGITGLQTGDEADYYCGTWDSSLSAGVVFGGGTKLTV |
| | L (SEQ ID NO: 970) |

In some embodiments, the nectin-4 antibody or conjugate comprises a heavy chain variable (VH) domain and a light chain variable (VL) domain, wherein the VH domain comprises CDR-H1, CDR-H2, and CDR-H3 sequences from a VH domain sequence selected from the group consisting of the sequences disclosed in Table 7, above. In some embodiments, the CDR-H1, CDR-H2, and CDR-H3 are according to Kabat. In some embodiments, the CDR-H1, CDR-H2, and CDR-H3 are according to Chothia. In some embodiments, the CDR-H1, CDR-H2, and CDR-H3 are according to IMGT.

In other embodiments, which may be combined with any of the foregoing embodiments, the nectin-4 antibody or conjugate comprises a heavy chain variable (VH) domain and a light chain variable (VL) domain, wherein the VL domain comprises CDR-L1, CDR-L2, and CDR-L3 sequences from a VL domain sequence selected from the group consisting of the sequences disclosed in Table 8, above. In some embodiments, the CDR-L1, CDR-L2, and CDR-L3 are according to Kabat. In some embodiments, the CDR-L1, CDR-L2, and CDR-L3 are according to Chothia. In some embodiments, the CDR-L1, CDR-L2, and CDR-L3 are according to IMGT.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:275, CDR-H2 comprises SEQ ID NO:276, CDR-H3 comprises SEQ ID NO:277, CDR-L1 comprises SEQ ID NO:272, CDR-L2 comprises SEQ ID NO:273, and CDR-L3 comprises SEQ ID NO:274.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:293, CDR-H2 comprises SEQ ID NO:294, CDR-H3 comprises SEQ ID NO:295, CDR-L1 comprises SEQ ID NO:290, CDR-L2 comprises SEQ ID NO:291, and CDR-L3 comprises SEQ ID NO:292.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:311, CDR-H2 comprises SEQ ID NO:312, CDR-H3 comprises SEQ ID NO:313, CDR-L1 comprises SEQ ID NO:308, CDR-L2 comprises SEQ ID NO:309, and CDR-L3 comprises SEQ ID NO:310.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:329, CDR-H2 comprises SEQ ID NO:330, CDR-H3 comprises SEQ ID NO:331, CDR-L1 comprises SEQ ID NO:326, CDR-L2 comprises SEQ ID NO:327, and CDR-L3 comprises SEQ ID NO:328.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:347, CDR-H2 comprises SEQ ID NO:348, CDR-H3 comprises SEQ ID NO:349, CDR-L1 comprises SEQ ID NO:344, CDR-L2 comprises SEQ ID NO:345, and CDR-L3 comprises SEQ ID NO:346.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:365, CDR-H2 comprises SEQ ID NO:366, CDR-H3 comprises SEQ ID NO:367, CDR-L1 comprises SEQ ID NO:362, CDR-L2 comprises SEQ ID NO:363, and CDR-L3 comprises SEQ ID NO:364.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:383, CDR-H2 comprises SEQ ID NO:384, CDR-H3 comprises SEQ ID NO:385, CDR-L1 comprises SEQ ID NO:380, CDR-L2 comprises SEQ ID NO:381, and CDR-L3 comprises SEQ ID NO:382.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:401, CDR-H2 comprises SEQ ID NO:402, CDR-H3 comprises SEQ ID NO:403, CDR-L1 comprises SEQ ID NO:398, CDR-L2 comprises SEQ ID NO:399, and CDR-L3 comprises SEQ ID NO:400.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:419, CDR-H2 comprises SEQ ID NO:420, CDR-H3 comprises SEQ ID NO:421, CDR-L1 comprises SEQ ID NO:416, CDR-L2 comprises SEQ ID NO:417, and CDR-L3 comprises SEQ ID NO:418.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:437, CDR-H2 comprises SEQ ID NO:438, CDR-H3 comprises SEQ ID NO:439, CDR-L1 comprises SEQ ID NO:434, CDR-L2 comprises SEQ ID NO:435, and CDR-L3 comprises SEQ ID NO:436.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:455, CDR-H2 comprises SEQ ID NO:456, CDR-H3 comprises SEQ ID NO:457, CDR-L1 comprises SEQ ID NO:452, CDR-L2 comprises SEQ ID NO:453, and CDR-L3 comprises SEQ ID NO:454.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:473, CDR-H2 comprises SEQ ID NO:474, CDR-H3 comprises SEQ ID NO:475, CDR-L1 comprises SEQ ID NO:470, CDR-L2 comprises SEQ ID NO:471, and CDR-L3 comprises SEQ ID NO:472.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:281, CDR-H2 comprises SEQ ID NO:282, CDR-H3 comprises SEQ ID NO:283, CDR-L1 comprises SEQ ID NO:278, CDR-L2 comprises SEQ ID NO:279, and CDR-L3 comprises SEQ ID NO:280.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:299, CDR-H2 comprises SEQ ID NO:300, CDR-H3 comprises SEQ ID NO:301, CDR-L1 comprises SEQ ID NO:296, CDR-L2 comprises SEQ ID NO:297, and CDR-L3 comprises SEQ ID NO:298.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:317, CDR-H2 comprises SEQ ID NO:318, CDR-H3 comprises SEQ ID NO:319, CDR-L1 comprises SEQ ID NO:314, CDR-L2 comprises SEQ ID NO:315, and CDR-L3 comprises SEQ ID NO:316.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:335, CDR-H2 comprises SEQ ID NO:336, CDR-H3 comprises SEQ ID NO:337, CDR-L1 comprises SEQ ID NO:332, CDR-L2 comprises SEQ ID NO:333, and CDR-L3 comprises SEQ ID NO:334.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:353, CDR-H2 comprises SEQ ID NO:354, CDR-H3 comprises SEQ ID NO:355, CDR-L1 comprises SEQ ID NO:350, CDR-L2 comprises SEQ ID NO:351, and CDR-L3 comprises SEQ ID NO:352.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:371, CDR-H2 comprises SEQ ID NO:372, CDR-H3 comprises SEQ ID NO:373, CDR-L1 comprises SEQ ID NO:368, CDR-L2 comprises SEQ ID NO:369, and CDR-L3 comprises SEQ ID NO:370.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:389, CDR-H2 comprises SEQ ID NO:390, CDR-H3 comprises SEQ ID NO:391, CDR-L1 comprises SEQ ID NO:386, CDR-L2 comprises SEQ ID NO:387, and CDR-L3 comprises SEQ ID NO:388.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:407, CDR-H2 comprises SEQ ID NO:408, CDR-H3 comprises SEQ ID NO:409, CDR-L1 comprises SEQ ID NO:404, CDR-L2 comprises SEQ ID NO:405, and CDR-L3 comprises SEQ ID NO:406.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:425, CDR-H2 comprises SEQ ID NO:426, CDR-H3 comprises SEQ ID NO:427, CDR-L1 comprises SEQ ID NO:422, CDR-L2 comprises SEQ ID NO:423, and CDR-L3 comprises SEQ ID NO:424.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:443, CDR-H2 comprises SEQ ID NO:444, CDR-H3 comprises SEQ ID NO:445, CDR-L1 comprises SEQ ID NO:440, CDR-L2 comprises SEQ ID NO:441, and CDR-L3 comprises SEQ ID NO:442.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:461, CDR-H2 comprises SEQ ID NO:462, CDR-H3 comprises SEQ ID NO:463, CDR-L1 comprises SEQ ID NO:458, CDR-L2 comprises SEQ ID NO:459, and CDR-L3 comprises SEQ ID NO:460.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:479, CDR-H2 comprises SEQ ID NO:480, CDR-H3 comprises SEQ ID NO:481, CDR-L1 comprises SEQ ID NO:476, CDR-L2 comprises SEQ ID NO:477, and CDR-L3 comprises SEQ ID NO:478.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:287, CDR-H2 comprises SEQ ID NO:288, CDR-H3 comprises SEQ ID NO:289, CDR-L1 comprises SEQ ID NO:284, CDR-L2 comprises SEQ ID NO:285, and CDR-L3 comprises SEQ ID NO:286.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:305, CDR-H2 comprises SEQ ID NO:306, CDR-H3 comprises SEQ ID NO:307, CDR-L1 comprises SEQ ID NO:302, CDR-L2 comprises SEQ ID NO:303, and CDR-L3 comprises SEQ ID NO:304.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:323, CDR-H2 comprises SEQ ID NO:324, CDR-H3 comprises SEQ ID NO:325, CDR-L1 comprises SEQ ID NO:320, CDR-L2 comprises SEQ ID NO:321, and CDR-L3 comprises SEQ ID NO:322.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:341, CDR-H2 comprises SEQ ID NO:342, CDR-H3 comprises SEQ ID NO:343, CDR-L1 comprises SEQ ID NO:338, CDR-L2 comprises SEQ ID NO:339, and CDR-L3 comprises SEQ ID NO:340.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:359, CDR-H2 comprises SEQ ID NO:360, CDR-H3 comprises SEQ ID NO:361, CDR-L1 comprises SEQ ID NO:356, CDR-L2 comprises SEQ ID NO:357, and CDR-L3 comprises SEQ ID NO:358.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:377, CDR-H2 comprises SEQ ID NO:378, CDR-H3 comprises SEQ ID NO:379, CDR-L1 comprises SEQ ID NO:374, CDR-L2 comprises SEQ ID NO:375, and CDR-L3 comprises SEQ ID NO:376.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:395, CDR-H2 comprises SEQ ID NO:396, CDR-H3 comprises SEQ ID NO:397, CDR-L1 comprises SEQ ID NO:392, CDR-L2 comprises SEQ ID NO:393, and CDR-L3 comprises SEQ ID NO:394.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:413, CDR-H2 comprises SEQ ID NO:414, CDR-H3 comprises SEQ ID NO:415, CDR-L1 comprises SEQ ID NO:410, CDR-L2 comprises SEQ ID NO:411, and CDR-L3 comprises SEQ ID NO:412.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:431, CDR-H2 comprises SEQ ID NO:432, CDR-H3 comprises SEQ ID NO:433, CDR-L1 comprises SEQ ID NO:428, CDR-L2 comprises SEQ ID NO:429, and CDR-L3 comprises SEQ ID NO:430.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:449, CDR-H2 comprises SEQ ID NO:450, CDR-H3 comprises SEQ ID NO:451, CDR-L1 comprises SEQ ID NO:446, CDR-L2 comprises SEQ ID NO:447, and CDR-L3 comprises SEQ ID NO:448.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:467, CDR-H2 comprises SEQ ID NO:468, CDR-H3 comprises SEQ ID NO:469, CDR-L1 comprises SEQ ID NO:464, CDR-L2 comprises SEQ ID NO:465, and CDR-L3 comprises SEQ ID NO:466.

In some embodiments, the nectin-4 antibody or conjugate comprises a VH domain and a VL domain, wherein the VH domain comprises CDR-H1, CDR-H2, and CDR-H3, and the VL domain comprises CDR-L1, CDR-L2, and CDR-L3, wherein CDR-H1 comprises SEQ ID NO:485, CDR-H2 comprises SEQ ID NO:486, CDR-H3 comprises SEQ ID NO:487, CDR-L1 comprises SEQ ID NO:482, CDR-L2 comprises SEQ ID NO:483, and CDR-L3 comprises SEQ ID NO:484.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:550, CDR-L2 comprises SEQ ID NO:551, and CDR-L3 comprises SEQ ID NO:552, wherein CDR-H1 comprises SEQ ID NO:553, CDR-H2 comprises SEQ ID NO:554, and CDR-H3 comprises SEQ ID NO:555.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:556, CDR-L2 comprises SEQ ID NO:557, and CDR-L3 comprises SEQ ID NO:558, wherein CDR-H1 comprises SEQ ID NO:559, CDR-H2 comprises SEQ ID NO:560, and CDR-H3 comprises SEQ ID NO:561.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:562, CDR-L2 comprises SEQ ID NO:563, and CDR-L3 comprises SEQ ID NO:564, wherein CDR-H1 comprises SEQ ID NO:565, CDR-H2 comprises SEQ ID NO:566, and CDR-H3 comprises SEQ ID NO:567.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:568, CDR-L2 comprises SEQ ID NO:569, and CDR-L3 comprises SEQ ID NO:570, wherein CDR-H1 comprises SEQ ID NO:571, CDR-H2 comprises SEQ ID NO:572, and CDR-H3 comprises SEQ ID NO:573.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:574, CDR-L2 comprises SEQ ID NO:575, and CDR-L3 comprises SEQ ID NO:576, wherein CDR-H1 comprises SEQ ID NO:577, CDR-H2 comprises SEQ ID NO:578, and CDR-H3 comprises SEQ ID NO:579.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:580, CDR-L2 comprises SEQ ID NO:581, and CDR-L3 comprises SEQ ID NO:582, wherein CDR-H1 comprises SEQ ID NO:583, CDR-H2 comprises SEQ ID NO:584, and CDR-H3 comprises SEQ ID NO:585.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:586, CDR-L2 comprises SEQ ID NO:587, and CDR-L3 comprises SEQ ID NO:588, wherein CDR-H1 comprises SEQ ID NO:589, CDR-H2 comprises SEQ ID NO:590, and CDR-H3 comprises SEQ ID NO:591.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:592, CDR-L2 comprises SEQ ID NO:593, and CDR-L3 comprises SEQ ID NO:594, wherein CDR-H1 comprises SEQ ID NO:595, CDR-H2 comprises SEQ ID NO:596, and CDR-H3 comprises SEQ ID NO:597.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:598, CDR-L2 comprises SEQ ID NO:599, and CDR-L3 comprises SEQ ID NO:600, wherein CDR-H1 comprises SEQ ID NO:601, CDR-H2 comprises SEQ ID NO:602, and CDR-H3 comprises SEQ ID NO:603.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:604, CDR-L2 comprises SEQ ID NO:605, and CDR-L3 comprises SEQ ID NO:606, wherein CDR-H1 comprises SEQ ID NO:607, CDR-H2 comprises SEQ ID NO:608, and CDR-H3 comprises SEQ ID NO:609.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:610, CDR-L2 comprises SEQ ID NO:611, and CDR-L3 comprises SEQ ID NO:612, wherein CDR-H1 comprises SEQ ID NO:613, CDR-H2 comprises SEQ ID NO:614, and CDR-H3 comprises SEQ ID NO:615.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:616, CDR-L2 comprises SEQ ID NO:617, and CDR-L3 comprises SEQ ID NO:618, wherein CDR-H1 comprises SEQ ID NO:619, CDR-H2 comprises SEQ ID NO:620, and CDR-H3 comprises SEQ ID NO:621.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:622, CDR-L2 comprises SEQ ID NO:623, and CDR-L3 comprises SEQ ID NO:624, wherein CDR-H1 comprises SEQ ID NO:625, CDR-H2 comprises SEQ ID NO:626, and CDR-H3 comprises SEQ ID NO:627.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:628, CDR-L2 comprises SEQ ID NO:629, and CDR-L3 comprises SEQ ID NO:630, wherein CDR-H1 comprises SEQ ID NO:631, CDR-H2 comprises SEQ ID NO:632, and CDR-H3 comprises SEQ ID NO:633.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:634, CDR-L2 comprises SEQ ID NO:635, and CDR-L3 comprises SEQ ID NO:636, wherein CDR-H1 comprises SEQ ID NO:637, CDR-H2 comprises SEQ ID NO:638, and CDR-H3 comprises SEQ ID NO:639.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:640, CDR-L2 comprises SEQ ID NO:641, and CDR-L3 comprises SEQ ID NO:642, wherein CDR-H1 comprises SEQ ID NO:643, CDR-H2 comprises SEQ ID NO:644, and CDR-H3 comprises SEQ ID NO:645.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:646, CDR-L2 comprises SEQ ID NO:647, and CDR-L3 comprises SEQ ID NO:648, wherein CDR-H1 comprises SEQ ID NO:649, CDR-H2 comprises SEQ ID NO:650, and CDR-H3 comprises SEQ ID NO:651.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:652, CDR-L2 comprises SEQ ID NO:653, and CDR-L3 comprises SEQ ID NO:654, wherein CDR-H1 comprises SEQ ID NO:655, CDR-H2 comprises SEQ ID NO:656, and CDR-H3 comprises SEQ ID NO:657.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:658, CDR-L2 comprises SEQ ID NO:659, and CDR-L3 comprises SEQ ID NO:660, wherein CDR-H1 comprises SEQ ID NO:661, CDR-H2 comprises SEQ ID NO:662, and CDR-H3 comprises SEQ ID NO:663.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:664, CDR-L2 comprises SEQ ID NO:665, and CDR-L3 comprises SEQ ID NO:666, wherein CDR-H1 comprises SEQ ID NO:667, CDR-H2 comprises SEQ ID NO:668, and CDR-H3 comprises SEQ ID NO:669.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:670, CDR-L2 comprises SEQ ID NO:671, and CDR-L3 comprises SEQ ID NO:672, wherein CDR-H1 comprises SEQ ID NO:673, CDR-H2 comprises SEQ ID NO:674, and CDR-H3 comprises SEQ ID NO:675.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:676, CDR-L2 comprises SEQ ID NO:677, and CDR-L3 comprises SEQ ID NO:678, wherein CDR-H1 comprises SEQ ID NO:679, CDR-H2 comprises SEQ ID NO:680, and CDR-H3 comprises SEQ ID NO:681.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:682, CDR-L2 comprises SEQ ID NO:683, and CDR-L3 comprises SEQ ID NO:684, wherein CDR-H1 comprises SEQ ID NO:685, CDR-H2 comprises SEQ ID NO:686, and CDR-H3 comprises SEQ ID NO:687.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:688, CDR-L2 comprises SEQ ID NO:689, and CDR-L3 comprises SEQ ID NO:690, wherein CDR-H1 comprises SEQ ID NO:691, CDR-H2 comprises SEQ ID NO:692, and CDR-H3 comprises SEQ ID NO:693.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:694, CDR-L2 comprises SEQ ID NO:695, and CDR-L3 comprises SEQ ID NO:696, wherein CDR-H1 comprises SEQ ID NO:697, CDR-H2 comprises SEQ ID NO:698, and CDR-H3 comprises SEQ ID NO:699.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:700, CDR-L2 comprises SEQ ID NO:701, and CDR-L3 comprises SEQ ID NO:702, wherein CDR-H1 comprises SEQ ID NO:703, CDR-H2 comprises SEQ ID NO:704, and CDR-H3 comprises SEQ ID NO:705.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:706, CDR-L2 comprises SEQ ID NO:707, and CDR-L3 comprises SEQ ID NO:708, wherein CDR-H1 comprises SEQ ID NO:709, CDR-H2 comprises SEQ ID NO:710, and CDR-H3 comprises SEQ ID NO:711.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:712, CDR-L2 comprises SEQ ID NO:713, and CDR-L3 comprises SEQ ID NO:714, wherein CDR-H1 comprises SEQ ID NO:715, CDR-H2 comprises SEQ ID NO:716, and CDR-H3 comprises SEQ ID NO:717.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:718, CDR-L2 comprises SEQ ID NO:719, and CDR-L3 comprises SEQ ID NO:720, wherein CDR-H1 comprises SEQ ID NO:721, CDR-H2 comprises SEQ ID NO:722, and CDR-H3 comprises SEQ ID NO:723.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:724, CDR-L2 comprises SEQ ID NO:725, and CDR-L3 comprises SEQ ID NO:726, wherein CDR-H1 comprises SEQ ID NO:727, CDR-H2 comprises SEQ ID NO:728, and CDR-H3 comprises SEQ ID NO:729.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:730, CDR-L2 comprises SEQ ID NO:731, and CDR-L3 comprises SEQ ID NO:732, wherein CDR-H1 comprises SEQ ID NO:733, CDR-H2 comprises SEQ ID NO:734, and CDR-H3 comprises SEQ ID NO:735.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:736, CDR-L2 comprises SEQ ID NO:737, and CDR-L3 comprises SEQ ID NO:738, wherein CDR-H1 comprises SEQ ID NO:739, CDR-H2 comprises SEQ ID NO:740, and CDR-H3 comprises SEQ ID NO:741.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:742, CDR-L2 comprises SEQ ID NO:743, and CDR-L3 comprises SEQ ID NO:744, wherein CDR-H1 comprises SEQ ID NO:745, CDR-H2 comprises SEQ ID NO:746, and CDR-H3 comprises SEQ ID NO:747.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:748, CDR-L2 comprises SEQ ID NO:749, and CDR-L3 comprises SEQ ID NO:750, wherein CDR-H1 comprises SEQ ID NO:751, CDR-H2 comprises SEQ ID NO:752, and CDR-H3 comprises SEQ ID NO:753.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:754, CDR-L2 comprises SEQ ID NO:755, and CDR-L3 comprises SEQ ID NO:756, wherein CDR-H1 comprises SEQ ID NO:757, CDR-H2 comprises SEQ ID NO:758, and CDR-H3 comprises SEQ ID NO:759.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:760, CDR-L2 comprises SEQ ID NO:761, and CDR-L3 comprises SEQ ID NO:762, wherein CDR-H1 comprises SEQ ID NO:763, CDR-H2 comprises SEQ ID NO:764, and CDR-H3 comprises SEQ ID NO:765.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:766, CDR-L2 comprises SEQ ID NO:767, and CDR-L3 comprises SEQ ID NO:768, wherein CDR-H1 comprises SEQ ID NO:769, CDR-H2 comprises SEQ ID NO:770, and CDR-H3 comprises SEQ ID NO:771.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:772, CDR-L2 comprises SEQ ID NO:773, and CDR-L3 comprises SEQ ID NO:774, wherein CDR-H1 comprises SEQ ID NO:775, CDR-H2 comprises SEQ ID NO:776, and CDR-H3 comprises SEQ ID NO:777.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:778, CDR-L2 comprises SEQ ID NO:779, and CDR-L3 comprises SEQ ID NO:780, wherein CDR-H1 comprises SEQ ID NO:781, CDR-H2 comprises SEQ ID NO:782, and CDR-H3 comprises SEQ ID NO:783.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:784, CDR-L2 comprises SEQ ID NO:785, and CDR-L3 comprises SEQ ID NO:786, wherein CDR-H1 comprises SEQ ID NO:787, CDR-H2 comprises SEQ ID NO:788, and CDR-H3 comprises SEQ ID NO:789.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:790, CDR-L2 comprises SEQ ID NO:791, and CDR-L3 comprises SEQ ID NO:792, wherein CDR-H1 comprises SEQ ID NO:793, CDR-H2 comprises SEQ ID NO:794, and CDR-H3 comprises SEQ ID NO:795.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:796, CDR-L2 comprises SEQ ID NO:797, and CDR-L3 comprises SEQ ID NO:798, wherein CDR-H1 comprises SEQ ID NO:799, CDR-H2 comprises SEQ ID NO:800, and CDR-H3 comprises SEQ ID NO:801.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:802, CDR-L2 comprises SEQ ID NO:803, and CDR-L3 comprises SEQ ID NO:804, wherein CDR-H1 comprises SEQ ID NO:805, CDR-H2 comprises SEQ ID NO:806, and CDR-H3 comprises SEQ ID NO:807.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:808, CDR-L2 comprises SEQ ID NO:809, and CDR-L3 comprises SEQ ID NO:810, wherein CDR-H1 comprises SEQ ID NO:811, CDR-H2 comprises SEQ ID NO:812, and CDR-H3 comprises SEQ ID NO:813.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:814, CDR-L2 comprises SEQ ID NO:815, and CDR-L3 comprises SEQ ID NO:816, wherein CDR-H1 comprises SEQ ID NO:817, CDR-H2 comprises SEQ ID NO:818, and CDR-H3 comprises SEQ ID NO:819.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:820, CDR-L2 comprises SEQ ID NO:821, and CDR-L3 comprises SEQ ID NO:822, wherein CDR-H1 comprises SEQ ID NO:823, CDR-H2 comprises SEQ ID NO:824, and CDR-H3 comprises SEQ ID NO:825.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:826, CDR-L2 comprises SEQ ID NO:827, and CDR-L3 comprises SEQ ID NO:828, wherein CDR-H1 comprises SEQ ID NO:829, CDR-H2 comprises SEQ ID NO:830, and CDR-H3 comprises SEQ ID NO: 831.

In some embodiments, the nectin-4 antibody or conjugate comprises a VL domain and a VH domain, wherein the VL domain comprises CDR-L1, CDR-L2, and CDR-L3, wherein CDR-L1 comprises SEQ ID NO:832, CDR-L2 comprises SEQ ID NO:833, and CDR-L3 comprises SEQ ID NO:834, wherein CDR-H1 comprises SEQ ID NO:835, CDR-H2 comprises SEQ ID NO:836, and CDR-H3 comprises SEQ ID NO:837.

TABLE 9
Nectin-4 antibody CDRs according to CHOTHIA

| Antibody | CDR-H1 | CDR-H2 | CDR-H3 | CDR-L1 | CDR-L2 | CDR-L3 |
|---|---|---|---|---|---|---|
| TNT-188; | GYTFTG | NPNSGG | GSALHFFGVFDY | RASQS | AASSL | QQSYSTPL |
| TNT-201; | Y (SEQ | (SEQ ID | (SEQ ID NO: 277) | ISSYLN | QS | T (SEQ ID |
| TNT-413; | ID | NO: 276) | | (SEQ ID | (SEQ | NO: 274) |
| TNT-412; | NO: 275) | | | NO: 272) | ID | |
| TNT-414 | | | | | NO: 273) | |
| TNT-189; | GGTFSS | IPIFGT | GYSSSWYEPYYFDY | RASQG | AAST | QQSYSTPY |
| TNT-202 | Y (SEQ | (SEQ ID | (SEQ ID NO: 295) | ISNYL | LQS | T (SEQ ID |
| | ID | NO: 294) | | A (SEQ | (SEQ | NO: 292) |
| | NO: 293) | | | ID | ID | |
| | | | | NO: 290) | NO: 291) | |
| TNT-190; | GFTFSD | SGSGGS | DEGYYYMDV (SEQ | RASQS | DASN | QQSYSTR |
| TNT-203 | Y (SEQ | (SEQ ID | ID NO: 313) | VSSYL | RAT | (SEQ ID |
| | ID | NO: 312) | | A (SEQ | (SEQ | NO: 310) |
| | NO: 311) | | | ID | ID | |
| | | | | NO: 308) | NO: 309) | |
| TNT-191; | GFTFSSY | SYDGSN | SSYYDYVWGSYRYT | RASQS | GASS | QQYNNWW |
| TNT-204 | (SEQ ID | (SEQ ID | DAFDI (SEQ ID | VSSSY | RAT | T (SEQ ID |
| | NO: 329) | NO: 330) | NO: 331) | LA (SEQ | (SEQ | NO: 328) |
| | | | | ID | ID | |
| | | | | NO: 326) | NO: 327) | |
| TNT-192; | GFTFSSY | SYDGSN | DYGYNWNFFDY | RASQG | AAST | QKYNSAPY |
| TNT-205 | (SEQ ID | (SEQ ID | (SEQ ID NO: 349) | ISNYL | LQS | T (SEQ ID |
| | NO: 347) | NO: 348) | | A (SEQ | (SEQ | NO: 346) |
| | | | | ID | ID | |
| | | | | NO: 344) | NO: 345) | |
| TNT-193; | GGSISSG | YYTGS | DPFPEPPDAFDI | RASQS | GASS | HQYGSSPL |
| TNT-206 | GY (SEQ | (SEQ ID | (SEQ ID NO: 367) | VSSNL | RAT | T (SEQ ID |
| | ID | NO: 366) | | A (SEQ | (SEQ | NO: 364) |
| | NO: 365) | | | ID | ID | |
| | | | | NO: 362) | NO: 363) | |
| TNT-194; | GYTFTS | IPIFGT | TAVAGFGIDY (SEQ | RASQG | AASSL | LQHDSFPIT |
| TNT-207; | Y (SEQ | (SEQ ID | ID NO: 385) | ISNYL | QS | (SEQ ID |
| TNT-422 | ID | NO: 384) | | A (SEQ | (SEQ | NO: 382) |
| | NO: 383) | | | ID | ID | |
| | | | | NO: 380) | NO: 381) | |
| TNT-195; | GGTFSS | IPIFGT | GYYYDSSGYYHQPY | RASQG | AASSL | QQSYSTPL |
| TNT-208; | Y (SEQ | (SEQ ID | YFDY (SEQ ID | ISNYL | QS | T (SEQ ID |
| TNT-423; | ID | NO: 402) | NO: 403) | A (SEQ | (SEQ | NO: 400) |
| TNT-417; | NO: 401) | | | ID | ID | |
| TNT-428 | | | | NO: 398) | NO: 399) | |
| TNT-196; | GFTFSD | SWNSGS | SGSWYLPLDY (SEQ | QASQD | AAST | QQSYSTPL |
| TNT-209 | Y (SEQ | (SEQ ID | ID NO: 421) | ISNYL | LQS | T (SEQ ID |
| | ID | NO: 420) | | N (SEQ | (SEQ | NO: 418) |
| | NO: 419) | | | ID | ID | |
| | | | | NO: 416) | NO: 417) | |
| TNT-197; | GFTFSSY | SYDGSN | ATLQLWLPLFDY | RASQS | GASS | QQYGSSLT |
| TNT-210; | (SEQ ID | (SEQ ID | (SEQ ID NO: 439) | VSSSY | RAT | (SEQ ID |
| TNT-424 | NO: 437) | NO: 438) | | LA | (SEQ | NO: 436) |
| | | | | (SEQ ID | ID | |
| | | | | NO: 434) | NO: 435) | |
| TNT-198; | GGTFSS | NPNSGG | GYYGDLYYFDY | RASQG | AAST | QQLNSHPI |
| TNT-211 | Y (SEQ | (SEQ ID | (SEQ ID NO: 457) | ISNYL | LQS | T (SEQ ID |
| | ID | NO: 456) | | A (SEQ | (SEQ | NO: 454) |
| | NO: 455) | | | ID | ID | |
| | | | | NO: 452) | NO: 453) | |
| TNT-153; | GGTFSS | SSSSST | AYYYGMDV (SEQ | RASQG | AAST | QQANSFPP |
| TNT-154 | Y (SEQ | (SEQ ID | ID NO: 475) | ISGWL | LQS | T (SEQ ID |
| TNT-212 | ID | NO: 474) | | A (SEQ | (SEQ | NO: 472) |
| | NO: 473) | | | ID | ID | |
| | | | | NO: 470) | NO: 471) | |
| TNT-317 | GGSISSY | YYSGN | DFHHYDGSAYRWYF | SGSSS | DNNK | GTWDSSLN |
| | (SEQ ID | (SEQ ID | DL | NIGNN | RPS | AGWV |
| | NO: 553) | NO: 554) | (SEQ ID NO: 555) | YVS | (SEQ | (SEQ ID |
| | | | | (SEQ ID | ID | NO: 552) |
| | | | | NO: 550) | NO: 551) | |
| TNT-318 | GGSISSN | HISES | EGEWKVLGAFDI | TLRSGI | YKSD | MIWHSSAS |
| | (SEQ ID | (SEQ ID | (SEQ ID NO: 573) | NVGSY | SDKQ | V |
| | NO: 571) | NO: 572) | | RIY | QGS | (SEQ ID |
| | | | | (SEQ ID | (SEQ | NO: 570) |
| | | | | NO: 568) | ID | |
| | | | | | NO: 569) | |
| TNT-320 | GYTFTV | SPNSGG | QTVVTPNWGFDY | SGSSS | GNYQ | ATWDDSL |
| | Y | (SEQ ID | (SEQ ID NO: 591) | NIGSN | RPS | NGWV |
| | (SEQ ID | NO: 590) | | TVH | (SEQ | (SEQ ID |
| | NO: 589) | | | (SEQ ID | ID | NO: 588) |
| | | | | NO: 586) | NO: 587) | |
| TNT-321 | GGSISSY | YYSGN | GGVGAQWDAFDI | RASQG | AASSL | LQHSFYPL |
| | (SEQ ID | (SEQ ID | (SEQ ID NO: 609) | ISNYL | QS | T |
| | NO: 607) | NO: 608) | | A | (SEQ | (SEQ ID |
| | | | | (SEQ ID | ID | NO: 606) |
| | | | | NO: 604) | NO: 605) | |
| TNT-322 | GGSISSY | YYSGN | SESYGYWFFYL | SGSSS | DNNK | GTWDSSLS |
| | (SEQ ID | (SEQ ID | (SEQ ID NO: 627) | NIGNN | RPS | AGVV |
| | NO: 625) | NO: 626) | | YVS | (SEQ | (SEQ ID |
| | | | | (SEQ ID | ID | NO: 624) |
| | | | | NO: 622) | NO: 623) | |
| TNT-323 | GGSISSY | YYIGT | SGNYYNDAFDI | RSSQS | KVSN | MQGTHWP |
| | (SEQ ID | (SEQ ID | (SEQ ID NO: 645) | LAYSD | RDS | LT |
| | NO: 643) | NO: 644) | | GNTYL | (SEQ | (SEQ ID |
| | | | | N | ID | NO: 642) |
| | | | | (SEQ ID | NO: 641) | |
| | | | | NO: 640) | | |
| TNT-324 | GYTFTG | NPNSGG | DTFYYYYGMDV | ASSTG | STSNK | LLYYDGA |
| | Y | (SEQ ID | (SEQ ID NO: 663) | AVTSG | HS | HL |
| | (SEQ ID | NO: 662) | | YYPN | (SEQ | (SEQ ID |
| | NO: 661) | | | (SEQ ID | ID | NO: 660) |
| | | | | NO: 658) | NO: 659) | |
| TNT-327 | GGSISDY | YTTGG | DRNWEWNFDL | TGTTS | EVSM | SSYAGSST |
| TNT-425; | (SEQ ID | (SEQ ID | (SEQ ID NO: 681) | DIGSY | SPS | VV |
| TNT-418; | NO: 679) | NO: 680) | | NLVS | (SEQ | (SEQ ID |
| TNT-419; | | | | (SEQ ID | ID | NO: 678) |
| TNT-429; | | | | NO: 676) | NO: 677) | |
| TNT-430 | | | | | | |
| TNT-328; | GGSMSS | YYSGN | SGNFYWNFDL | TGSSS | GNSN | QSYDSSLS |
| TNT-426; | Y | (SEQ ID | (SEQ ID NO: 699) | NIGAG | RPS | GSAV |
| TNT-420; | (SEQ ID | NO: 698) | | YNVH | (SEQ | (SEQ ID |
| TNT-431 | NO: 697) | | | (SEQ ID | ID | NO: 696) |
| | | | | NO: 694) | NO: 695) | |
| TNT-330 | GFTFSSY | SISSSY | DRGLDDAFDI | RSSQS | TLSYR | MQRIEFPY |
| | (SEQ ID | (SEQ ID | (SEQ ID NO: 717) | LLDSD | AS | T |
| | NO: 715) | NO: 716) | | DGNTY | (SEQ | (SEQ ID |
| | | | | LD | ID | NO: 714) |
| | | | | (SEQ ID | NO: 713) | |
| | | | | NO: 712) | | |
| TNT-331 | GGSIRSY | YTSGS | ENWGPSFDY | SGSSS | DNNK | GTWDTSLS |
| | (SEQ ID | (SEQ ID | (SEQ ID NO: 735) | NIGNN | RPS | SGWL |
| | NO: 733) | NO: 734) | | YVS | (SEQ | (SEQ ID |
| | | | | (SEQ ID | ID | NO: 732) |
| | | | | NO: 730) | NO: 731) | |
| TNT-332 | GYTFTN | NPTLDS | SAGFDAFDI | RASQG | SASTL | QQLNSYPF |
| | Y | (SEQ ID | (SEQ ID NO: 753) | INSYL | QG | T |
| | (SEQ ID | NO: 752) | | A | (SEQ | (SEQ ID |
| | NO: 751) | | | (SEQ ID | ID | NO: 750) |
| | | | | NO: 748) | NO: 749) | |
| TNT-333; | GGSISSY | FYIGN | SYPYWYFDL | SGSSS | DNNK | GTWDSSLS |
| TNT-427; | (SEQ ID | (SEQ ID | (SEQ ID NO: 771) | NIGNN | RPS | AGVV |
| TNT-421; | NO: 769) | NO: 770) | | YVS | (SEQ | (SEQ ID |
| TNT-432 | | | | (SEQ ID | ID | NO: 768) |
| | | | | NO: 766) | NO: 767) | |
| TNT-334 | GFTFSR | NSDGSS | DNWYFDY | RASQS | GVST | QQYNNWP |
| | Y | (SEQ ID | (SEQ ID NO: 789) | VSINL | RAT | RT |
| | (SEQ ID | NO: 788) | | A | (SEQ | (SEQ ID |
| | NO: 787) | | | (SEQ ID | ID | NO: 786) |
| | | | | NO: 784) | NO: 785) | |
| TNT-335 | GGSISSY | HYSGI | GSYWFDP | SGSSS | SNHQ | AAWEDSLS |
| | (SEQ ID | (SEQ ID | (SEQ ID NO: 807) | NIGSD | RPS | GPI |
| | NO: 805) | NO: 806) | | YIY | (SEQ | (SEQ ID |
| | | | | (SEQ ID | ID | NO: 804) |
| | | | | NO: 802) | NO: 803) | |
| TNT-336 | GFTFSN | NNGGTS | DWYFDL | TLSSG | VNSQ | QSWDTDI |
| | Y | (SEQ ID | (SEQ ID NO: 825) | HSFYA | GSHN | WV |
| | (SEQ ID | NO: 824) | | IA | KGD | (SEQ ID |
| | NO: 823) | | | (SEQ ID | (SEQ | NO: 822) |
| | | | | NO: 820) | ID | |
| | | | | | NO: 821) | |
| TNT-337 | GFTFSN | SSGGSY | QELGSYYAMDY | RSSQSI | KVSN | FQGSHVPY |
| | Y | (SEQ ID | (SEQ ID NO: 843) | VHSNA | RFS | T |
| | (SEQ ID | NO: 842) | | NTYLE | (SEQ | (SEQ ID |
| | NO: 841) | | | (SEQ ID | ID | NO: 840) |
| | | | | NO: 838) | NO: 839) | |

TABLE 10
Nectin-4 antibody CDRs according to KABAT

| Antibody | CDR-H1 | CDR-H2 | CDR-H3 | CDR-L1 | CDR-L2 | CDR-L3 |
|---|---|---|---|---|---|---|
| TNT-188; | GYYMH | WINPNS | GSALHFFGVFDY | RASQS | AASSL | QQSYSTPL |
| TNT-201; | (SEQ ID | GGTNYA | (SEQ ID NO: 283) | ISSYLN | QS | T (SEQ ID |
| TNT-413; | NO: 281) | QKFQG | | (SEQ ID | (SEQ | NO: 280) |
| TNT-412; | | (SEQ ID | | NO: 278 | ID | |
| TNT-414 | | NO: 282) | | | NO: 279) | |
| TNT-189; | SYAIS | GIIPIFGT | GYSSSWYEPYYFDY | RASQG | AAST | QQSYSTPY |
| TNT-202 | (SEQ ID | ANYAQ | (SEQ ID NO: 301) | ISNYL | LQS | T (SEQ ID |
| | NO: 299) | KFQG | | A (SEQ | (SEQ | NO: 298) |
| | | (SEQ ID | | ID | ID | |
| | | NO: 300) | | NO: 296) | NO: 297) | |
| TNT-190; | DYYMS | AISGSG | DEGYYYMDV (SEQ | RASQS | DASN | QQSYSTR |
| TNT-203 | (SEQ ID | GSTYYA | ID NO: 319) | VSSYL | RAT | (SEQ ID |
| | NO: 317) | DSVKG | | A (SEQ | (SEQ | NO: 316) |
| | | (SEQ ID | | ID | ID | |
| | | NO: 318) | | NO: 314) | NO: 315) | |
| TNT-191; | SYGMH | VISYDG | SSYYDYVWGSYRYT | RASQS | GASS | QQYNNWW |
| TNT-204 | (SEQ ID | SNKYYA | DAFDI (SEQ ID | VSSSY | RAT | T (SEQ ID |
| | NO: 335) | DSVKG | NO: 337) | LA | (SEQ | NO: 334) |
| | | (SEQ ID | | (SEQ ID | ID | |
| | | NO: 336) | | NO: 332) | NO: 333) | |
| TNT-192; | SYGMH | VISYDG | DYGYNWNFFDY | RASQG | AAST | QKYNSAPY |
| TNT-205 | (SEQ ID | SNKYYA | (SEQ ID NO: 355) | ISNYL | LQS | T (SEQ ID |
| | NO: 353) | DSVKG | | A (SEQ | (SEQ | NO: 352) |
| | | (SEQ ID | | ID | ID | |
| | | NO: 354) | | NO: 350) | NO: 351) | |
| TNT-193; | SGGYY | SIYYTGS | DPFPEPPDAFDI | RASQS | GASS | HQYGSSPL |
| TNT-206 | WA (SEQ | TYYNPS | (SEQ ID NO: 373) | VSSNL | RAT | T (SEQ ID |
| | ID | LKS | | A (SEQ | (SEQ | NO: 370) |
| | NO: 371) | (SEQ ID | | ID | ID | |
| | | NO: 372) | | NO: 368) | NO: 369) | |
| TNT-194; | SYDIN | GIIPIFGT | TAVAGFGIDY (SEQ | RASQG | AASSL | LQHDSFPIT |
| TNT-207; | (SEQ ID | ANYAQ | ID NO: 391) | ISNYL | QS | (SEQ ID |
| TNT-422 | NO: 389) | KFQG | | A (SEQ | (SEQ | NO: 388) |
| | | (SEQ ID | | ID | ID | |
| | | NO: 390) | | NO: 386) | NO: 387) | |
| TNT-195; | SYAIS | GIIPIFGT | GYYYDSSGYYHQPY | RASQG | AASSL | QQSYSTPL |
| TNT-208; | (SEQ ID | ANYAQ | YFDY (SEQ ID | ISNYL | QS | T (SEQ ID |
| TNT-423; | NO: 407) | KFQG | NO: 409) | A (SEQ | (SEQ | NO: 406) |
| TNT-417; | | (SEQ ID | | ID | ID | |
| TNT-428 | | NO: 408) | | NO: 404) | NO: 405) | |
| TNT-196; | DYYMS | GISWNS | SGSWYLPLDY (SEQ | QASQD | AAST | QQSYSTPL |
| TNT-209 | (SEQ ID | GSIGYA | ID NO: 427) | ISNYL | LQS | T (SEQ ID |
| | NO: 425) | DSVKG | | N (SEQ | (SEQ | NO: 424) |
| | | (SEQ ID | | ID | ID | |
| | | NO: 426) | | NO: 422) | NO: 423) | |
| TNT-197; | SYAMH | VISYDG | ATLQLWLPLFDY | RASQS | GASS | QQYGSSLT |
| TNT-210; | (SEQ ID | SNKYYA | (SEQ ID NO: 445) | VSSSY | RAT | (SEQ ID |
| TNT-424 | NO: 443) | DSVKG | | LA | (SEQ | NO: 442) |
| | | (SEQ ID | | (SEQ ID | ID | |
| | | NO: 444) | | NO: 440) | NO: 441) | |
| TNT-198; | SYAIS | WINPNS | GYYGDLYYFDY | RASQG | AAST | QQLNSHPI |
| TNT-211 | (SEQ ID | GGTNYA | (SEQ ID NO: 463) | ISNYL | LQS | T (SEQ ID |
| | NO: 461) | QKFQG | | A (SEQ | (SEQ | NO: 460) |
| | | (SEQ ID | | ID | ID | |
| | | NO: 462) | | NO: 458) | NO: 459) | |
| TNT-153; | SYNMN | YISSSSS | AYYYGMDV (SEQ ID | RASQG | AAST | QQANSFPP |
| TNT-154 | (SEQ ID | TIYYAD | NO: 481) | ISGWL | LQS | T (SEQ ID |
| TNT-212 | NO: 479) | SVKG | | A (SEQ | (SEQ | NO: 478) |
| | | (SEQ ID | | ID | ID | |
| | | NO: 480) | | NO: 476) | NO: 477) | |
| TNT-317 | SYFWS | YIYYSG | DFHHYDGSAYRWYF | SGSSS | DNNK | GTWDSSLN |
| | (SEQ ID | NTNYNP | DL | NIGNN | RPS | AGWV |
| | NO: 559) | SLKS | (SEQ ID NO: 561) | YVS | (SEQ | (SEQ ID |
| | | (SEQ ID | | (SEQ ID | ID | NO: 558) |
| | | NO: 560) | | NO: 556) | NO: 557) | |
| TNT-318 | SNYWT | RIHISES | EGEWKVLGAFDI | TLRSGI | SDKQ | MIWHSSAS |
| | (SEQ ID | TNYNPS | (SEQ ID NO: 579) | NVGSY | QGS | V |
| | NO: 577) | LKS | | RIY | (SEQ | (SEQ ID |
| | | (SEQ ID | | (SEQ ID | ID | NO: 576) |
| | | NO: 578) | | NO: 574) | NO: 575) | |
| TNT-320 | VYYIH | WISPNS | QTVVTPNWGFDY | SGSSS | GNYQ | ATWDDSL |
| | (SEQ ID | GGTNYA | (SEQ ID NO: 597) | NIGSN | RPS | NGWV |
| | NO: 595) | QKFQG | | TVH | (SEQ | (SEQ ID |
| | | (SEQ ID | | (SEQ ID | ID | NO: 594) |
| | | NO: 596) | | NO: 592) | NO: 593) | |
| TNT-321 | SYYWS | YIYYSG | GGVGAQWDAFDI | RASQG | AASSL | LQHSFYPL |
| | (SEQ ID | NVNYNP | (SEQ ID NO: 615) | ISNYL | QS | T |
| | NO: 613) | SLKS | | A | (SEQ | (SEQ ID |
| | | (SEQ ID | | (SEQ ID | ID | NO: 612) |
| | | NO: 614) | | NO: 610) | NO: 611) | |
| TNT-322 | SYYWN | YIYYSG | SESYGYWFFYL | SGSSS | DNNK | GTWDSSLS |
| | (SEQ ID | NTNYNP | (SEQ ID NO: 633) | NIGNN | RPS | AGVV |
| | NO: 631) | SLKS | | YVS | (SEQ | (SEQ ID |
| | | (SEQ ID | | (SEQ ID | ID | NO: 630) |
| | | NO: 632) | | NO: 628) | NO: 629) | |
| TNT-323 | SYFWN | YLYYIG | SGNYYNDAFDI | RSSQS | KVSN | MQGTHWP |
| | (SEQ ID | TTNYNP | (SEQ ID NO: 651) | LAYSD | RDS | LT |
| | NO: 649) | SLKS | | GNTYL | (SEQ | (SEQ ID |
| | | (SEQ ID | | N | ID | NO: 648) |
| | | NO: 650) | | (SEQ ID | NO: 647) | |
| | | | | NO: 646) | | |
| TNT-324 | GYFIH | WINPNS | DTFYYYYGMDV | ASSTG | STSNK | LLYYDGA |
| | (SEQ ID | GGTNYA | (SEQ ID NO: 669) | AVTSG | HS | HL |
| | NO: 667) | QKFQG | | YYPN | (SEQ | (SEQ ID |
| | | (SEQ ID | | (SEQ ID | ID | NO: 666) |
| | | NO: 668) | | NO: 664) | NO: 665) | |
| TNT-327; | DYYWI | RIYTTG | DRNWEWNFDL | TGTTS | EVSM | SSYAGSST |
| TNT-425; | (SEQ ID | GAKYNP | (SEQ ID NO: 687) | DIGSY | SPS | VV |
| TNT-418; | NO: 685) | SLKS | | NLVS | (SEQ | (SEQ ID |
| TNT-419; | | (SEQ ID | | (SEQ ID | ID | NO: 684) |
| TNT-429; | | NO: 686) | | NO: 682) | NO: 683) | |
| TNT-430 | | | | | | |
| TNT-328; | SYFWS | YMYYS | SGNFYWNFDL | TGSSS | GNSN | QSYDSSLS |
| TNT-426; | (SEQ ID | GNTNYN | (SEQ ID NO: 705) | NIGAG | RPS | GSAV |
| TNT-420; | NO: 703) | PSLKS | | YNVH | (SEQ | (SEQ ID |
| TNT-431 | | (SEQ ID | | (SEQ ID | ID | NO: 702) |
| | | NO: 704) | | NO: 700) | NO: 701) | |
| TNT-330 | SYNMN | SISISSSY | DRGLDDAFDI | RSSQS | TLSYR | MQRIEFPY |
| | (SEQ ID | IYYADS | (SEQ ID NO: 723) | LLDSD | AS | T |
| | NO: 721) | VKG | | DGNTY | (SEQ | (SEQ ID |
| | | (SEQ ID | | LD | ID | NO: 720) |
| | | NO: 722) | | (SEQ ID | NO: 719) | |
| | | | | NO: 718) | | |
| TNT-331 | SYYWS | RIYTSGS | ENWGPSFDY | SGSSS | DNNK | GTWDTSLS |
| | (SEQ ID | TNYNPS | (SEQ ID NO: 741) | NIGNN | RPS | SGWL |
| | NO: 739) | LKS | | YVS | (SEQ | (SEQ ID |
| | | (SEQ ID | | (SEQ ID | ID | NO: 738) |
| | | NO: 740) | | NO: 736) | NO: 737) | |
| TNT-332 | NYYIH | IINPTLD | SAGFDAFDI | RASQG | SASTL | QQLNSYPF |
| | (SEQ ID | STSYAQ | (SEQ ID NO: 759) | INSYL | QG | T |
| | NO: 757) | KFQD | | A | (SEQ | (SEQ ID |
| | | (SEQ ID | | (SEQ ID | ID | NO: 756) |
| | | NO: 758) | | NO: 754) | NO: 755) | |
| TNT-333; | SYYWS | YIFYIGN | SYPYWYFDL | SGSSS | DNNK | GTWDSSLS |
| TNT-427; | (SEQ ID | TNYYPS | (SEQ ID NO: 777) | NIGNN | RPS | AGVV |
| TNT-421; | NO: 775) | LKS | | YVS | (SEQ | (SEQ ID |
| TNT-432 | | (SEQ ID | | (SEQ ID | ID | NO: 774) |
| | | NO: 776) | | NO: 772) | NO: 773) | |
| TNT-334 | RYWMH | RINSDGS | DNWYFDY | RASQS | GVST | QQYNNWP |
| | (SEQ ID | SSNYAD | (SEQ ID NO: 795) | VSINL | RAT | RT |
| | NO: 793) | SVKG | | A | (SEQ | (SEQ ID |
| | | (SEQ ID | | (SEQ ID | ID | NO: 792) |
| | | NO: 794) | | NO: 790) | NO: 791) | |
| TNT-335 | SYYWS | YIHYSGI | GSYWFDP | SGSSS | SNHQ | AAWEDSLS |
| | (SEQ ID | SNYNPS | (SEQ ID NO: 813) | NIGSD | RPS | GPI |
| | NO: 811) | LKS | | YIY | (SEQ | (SEQ ID |
| | | (SEQ ID | | (SEQ ID | ID | NO: 810) |
| | | NO: 812) | | NO: 808) | NO: 809) | |
| TNT-336 | NYWMH | RLNNGG | DWYFDL | TLSSG | GSHN | QSWDTDI |
| | (SEQ ID | TSTRYA | (SEQ ID NO: 831) | HSFYA | KGD | WV |
| | NO: 829) | DSVKS | | IA | (SEQ | (SEQ ID |
| | | (SEQ ID | | (SEQ ID | ID | NO: 828) |
| | | NO: 830) | | NO: 826) | NO: 827) | |
| TNT-337 | NYDMS | TISSGGS | QELGSYYAMDY | RSSQSI | KVSN | FQGSHVPY |
| | (SEQ ID | YTYYVD | (SEQ ID NO: 849) | VHSNA | RFS | T |
| | NO: 847) | SVKG | | NTYLE | (SEQ | (SEQ ID |
| | | (SEQ ID | | (SEQ | ID | NO: 846) |
| | | NO: 848) | | ID | NO: 845) | |
| | | | | NO: 844) | | |

TABLE 11
Nectin-4 antibody CDRs according to IMGT

| Antibody | CDR-H1 | CDR-H2 | CDR-H3 | CDR-L1 | CDR-L2 | CDR-L3 |
|---|---|---|---|---|---|---|
| TNT-188; | GYTFTG | INPNSG | ARGSALHFFGVFDY | QSISSY | AAS | QQSYSTPL |
| TNT-201; | YY (SEQ | GT (SEQ | (SEQ ID NO: 289) | (SEQ ID | (SEQ | T (SEQ ID |
| TNT-413; | ID | ID | | NO: 284 | ID | NO: 286) |
| TNT-412; | NO: 287) | NO: 288) | | | NO: 285) | |
| TNT-414 | | | | | | |
| TNT-189; | GGTFSS | IIPIFGTA | ARGYSSSWYEPYYF | QGISN | AAS | QQSYSTPY |
| TNT-202 | YA (SEQ | (SEQ ID | DY | Y (SEQ | (SEQ | T (SEQ ID |
| | ID | NO: 306) | (SEQ ID NO: 307) | ID | ID | NO: 304) |
| | NO: 305) | | | NO: 302) | NO: 303) | |
| TNT-190; | GFTFSD | ISGSGGS | ARDEGYYYMDV | QSVSS | DAS | QQSYSTR |
| TNT-203 | YY (SEQ | T (SEQ | (SEQ ID NO: 325) | Y (SEQ | (SEQ | (SEQ ID |
| | ID | ID | | ID | ID | NO: 322) |
| | NO: 323) | NO: 324) | | NO: 320) | NO: 321) | |
| TNT-191; | GFTFSSY | ISYDGS | AKSSYYDYVWGSYR | QSVSS | GAS | QQYNNWW |
| TNT-204 | G (SEQ | NK (SEQ | YTDAFDI (SEQ ID | SY | (SEQ | T (SEQ ID |
| | ID | ID | NO: 343) | (SEQ ID | ID | NO: 340) |
| | NO: 341) | NO: 342) | | NO: 338) | NO: 339) | |
| TNT-192; | GFTFSSY | ISYDGS | AKDYGYNWNFFDY | QGISN | AAS | QKYNSAPY |
| TNT-205 | G (SEQ | NK (SEQ | (SEQ ID NO: 361) | Y (SEQ | (SEQ | T (SEQ ID |
| | ID | ID | | ID | ID | NO: 358) |
| | NO: 359) | NO: 360) | | NO: 356) | NO: 357) | |
| TNT-193; | GGSISSG | IYYTGST | ARDPFPEPPDAFDI | QSVSS | GAS | HQYGSSPL |
| TNT-206 | GYY | (SEQ ID | (SEQ ID NO: 379) | N (SEQ | (SEQ | T (SEQ ID |
| | (SEQ ID | NO: 378) | | ID | ID | NO: 376) |
| | NO: 377) | | | NO: 374) | NO: 375) | |
| TNT-194; | GYTFTS | IIPIFGTA | ASTAVAGFGIDY | QGISN | AAS | LQHDSFPIT |
| TNT-207; | YD (SEQ | (SEQ ID | (SEQ ID NO: 397) | Y (SEQ | (SEQ | (SEQ ID |
| TNT-422 | ID | NO: 396) | | ID | ID | NO: 394) |
| | NO: 395) | | | NO: 392) | NO: 393) | |
| TNT-195; | GGTFSS | IIPIFGTA | AAGYYYDSSGYYHQ | QGISN | AAS | QQSYSTPL |
| TNT-208; | YA (SEQ | (SEQ ID | PYYFDY (SEQ ID | Y (SEQ | (SEQ | T (SEQ ID |
| TNT-423; | ID | NO: 414) | NO: 415) | ID | ID | NO: 412) |
| TNT-417; | NO: 413) | | | NO: 410) | NO: 411) | |
| TNT-428 | | | | | | |
| TNT-196; | GFTFSD | ISWNSG | ASSGSWYLPLDY | QDISN | AAS | QQSYSTPL |
| TNT-209 | YY (SEQ | SI (SEQ | (SEQ ID NO: 433) | Y (SEQ | (SEQ | T (SEQ ID |
| | ID | ID | | ID | ID | NO: 430) |
| | NO: 431) | NO: 432) | | NO: 428) | NO: 429) | |
| TNT-197; | GFTFSSY | ISYDGS | ARATLQLWLPLFDY | QSVSS | GAS | QQYGSSLT |
| TNT-210; | A (SEQ | NK (SEQ | (SEQ ID NO: 451) | SY | (SEQ | (SEQ ID |
| TNT-424 | ID | ID | | (SEQ ID | ID | NO: 448) |
| | NO: 449) | NO: 450) | | NO: 446) | NO: 447) | |
| TNT-198; | GGTFSS | INPNSG | ATGYYGDLYYFDY | QGISN | AAS | QQLNSHPI |
| TNT-211 | YA (SEQ | GT (SEQ | (SEQ ID NO: 469) | Y (SEQ | (SEQ | T (SEQ ID |
| | ID | ID | | ID | ID | NO: 466) |
| | NO: 467) | NO: 468) | | NO: 464) | NO: 465) | |
| TNT-153; | GFTFSSY | ISSSSSTI | ARAYYYGMDV (SEQ | QGISG | AAS | QQANSFPP |
| TNT-154 | N (SEQ | (SEQ ID | ID NO: 487) | W (SEQ | (SEQ | T (SEQ ID |
| TNT-212 | ID | NO: 486) | | ID | ID | NO: 484) |
| | NO: 485) | | | NO: 482) | NO: 483) | |
| TNT-317 | GGSISSY | IYYSGN | ARDFHHYDGSAYRW | SSNIG | DNN | GTWDSSLN |
| | F | T | YFDL | NNY | (SEQ | AGWV |
| | (SEQ ID | (SEQ ID | (SEQ ID NO: 567) | (SEQ ID | ID | (SEQ ID |
| | NO: 565) | NO: 566) | | NO: 562) | NO: 563) | NO: 564) |
| TNT-318 | GGSISSN | IHISEST | AREGEWKVLGAFDI | SGINV | YKSD | MIWHSSAS |
| | Y | (SEQ ID | (SEQ ID NO: 585) | GSYR | SDK | V |
| | (SEQ ID | NO: 584) | | (SEQ ID | (SEQ | (SEQ ID |
| | NO: 583) | | | NO: 580) | ID | NO: 582) |
| | | | | | NO: 581) | |
| TNT-320 | GYTFTV | ISPNSGG | ARQTVVTPNWGFDY | SSNIGS | GNY | ATWDDSL |
| | (SEQ ID | T | (SEQ ID NO: 603) | NT | (SEQ | NGWV |
| | NO: 601) | (SEQ ID | | (SEQ ID | ID | (SEQ ID |
| | | NO: 602) | | NO: 598) | NO: 599) | NO: 600) |
| TNT-321 | GGSISSY | IYYSGN | AIGGVGAQWDAFDI | QGISN | AAS | LQHSFYPL |
| | Y | V | (SEQ ID NO: 621) | Y | (SEQ | T |
| | (SEQ ID | (SEQ ID | | (SEQ ID | ID | (SEQ ID |
| | NO: 619) | NO: 620) | | NO: 616) | NO: 617) | NO: 618) |
| TNT-322 | GGSISSY | IYYSGN | ARSESYGYWFFYL | SSNIG | DNN | GTWDSSLS |
| | Y | T | (SEQ ID NO: 639) | NNY | (SEQ | AGVV |
| | (SEQ ID | (SEQ ID | | (SEQ ID | ID | (SEQ ID |
| | NO: 637) | NO: 638) | | NO: 634) | NO: 635) | NO: 636) |
| TNT-323 | GGSISSY | LYYIGT | ARSGNYYNDAFDI | QSLAY | KVS | MQGTHWP |
| | F | T | (SEQ ID NO: 657) | SDGNT | (SEQ | LT |
| | (SEQ ID | (SEQ ID | | Y | ID | (SEQ ID |
| | NO: 655) | NO: 656) | | (SEQ ID | NO: 653) | NO: 654) |
| | | | | NO: 652) | | |
| TNT-324 | GYTFTG | INPNSG | ARDTFYYYYGMDV | TGAVT | STS | LLYYDGA |
| | YF | GT | (SEQ ID NO: 675) | SGYY | (SEQ | HL |
| | (SEQ ID | (SEQ ID | | (SEQ ID | ID | (SEQ ID |
| | NO: 673) | NO: 674) | | NO: 670) | NO: 671) | NO: 672) |
| TNT-327; | GGSISDY | IYTTGG | ARDRNWEWNFDL | TSDIGS | EVS | SSYAGSST |
| TNT-425; | Y | A | (SEQ ID NO: 693) | YNL | (SEQ | VV |
| TNT-418; | (SEQ ID | (SEQ ID | | (SEQ ID | ID | (SEQ ID |
| TNT-419; | NO: 691) | NO: 692) | | NO: 688) | NO: 689) | NO: 690) |
| TNT-429; | | | | | | |
| TNT-430 | | | | | | |
| TNT-328; | GGSMSS | MYYSG | ARSGNFYWNFDL | SSNIG | GNS | QSYDSSLS |
| TNT-426; | YF | NT | (SEQ ID NO: 711) | AGYN | (SEQ | GSAV |
| TNT-420; | (SEQ ID | (SEQ ID | | (SEQ ID | ID | (SEQ ID |
| TNT-431 | NO: 709) | NO: 710) | | NO: 706) | NO: 707) | NO: 708) |
| TNT-330 | GFTFSSY | ISISSSYI | ARDRGLDDAFDI | QSLLD | TLS | MQRIEFPY |
| | N | (SEQ ID | (SEQ ID NO: 729) | SDDGN | (SEQ | T |
| | (SEQ ID | NO: 728) | | TY | ID | (SEQ ID |
| | NO: 727) | | | (SEQ ID | NO: 725) | NO: 726) |
| | | | | NO: 724) | | |
| TNT-331 | GGSIRSY | IYTSGST | AKENWGPSFDY | SSNIG | DNN | GTWDTSLS |
| | Y | (SEQ ID | (SEQ ID NO: 747) | NNY | (SEQ | SGWL |
| | (SEQ ID | NO: 746) | | (SEQ ID | ID | (SEQ ID |
| | NO: 745) | | | NO: 742) | NO: 743) | NO: 744) |
| TNT-332 | GYTFTN | INPTLDS | ARSAGFDAFDI | QGINS | SAS | QQLNSYPF |
| | YY | T | (SEQ ID NO: 765) | Y | (SEQ | T |
| | (SEQ ID | (SEQ ID | | (SEQ ID | ID | (SEQ ID |
| | NO: 763) | NO: 764) | | NO: 760) | NO: 761) | NO: 762) |
| TNT-333; | GGSISSY | IFYIGNT | ARSYPYWYFDL | SSNIG | DNN | GTWDSSLS |
| TNT-427; | Y | (SEQ ID | (SEQ ID NO: 783) | NNY | (SEQ | AGVV |
| TNT-421; | (SEQ ID | NO: 782) | | (SEQ ID | ID | (SEQ ID |
| TNT-432 | NO: 781) | | | NO: 778) | NO: 779) | NO: 780) |
| TNT-334 | GFTFSR | INSDGSS | VRDNWYFDY | QSVSI | GVS | QQYNNWP |
| | YW | S | (SEQ ID NO: 801) | N | (SEQ | RT |
| | (SEQ ID | (SEQ ID | | (SEQ ID | ID | (SEQ ID |
| | NO: 799) | NO: 800) | | NO: 796) | NO: 797) | NO: 798) |
| TNT-335 | GGSISSY | IHYSGIS | ARGSYWFDP | SSNIGS | SNH | AAWEDSLS |
| | Y | (SEQ ID | (SEQ ID NO: 819) | DY | (SEQ | GPI |
| | (SEQ ID | NO: 818) | | (SEQ ID | ID | (SEQ ID |
| | NO: 817) | | | NO: 814) | NO: 815) | NO: 816) |
| TNT-336 | GFTFSN | LNNGGT | VRDWYFDL | SGHSF | VNSQ | QSWDTDI |
| | YW | ST | (SEQ ID NO: 837) | YA | GSH | WV |
| | (SEQ ID | (SEQ ID | | (SEQ ID | (SEQ | (SEQ ID |
| | NO: 835) | NO: 836) | | NO: 832) | ID | NO: 834) |
| | | | | | NO: 833) | |
| TNT-337 | GFTFSN | ISSGGSY | ARQELGSYYAMDY | QSIVH | KVS | FQGSHVPY |
| | YD (SEQ | T (SEQ | (SEQ ID NO: 855) | SNANT | (SEQ | T |
| | ID | ID | | Y (SEQ | ID | (SEQ ID |
| | NO: 853) | NO: 854) | | ID | NO: 851) | NO: 852) |
| | | | | NO: 850) | | |

In some embodiments, the nectin-4 antibody or conjugate comprises a light chain comprising a sequence selected from the group consisting of the sequences disclosed in Table 12, below. In some embodiments, the nectin-4 antibody comprises a heavy chain comprising a sequence selected from the group consisting of the sequences disclosed in Table 13, below. Table 12. Nectin-4 antibody mouse IgG2a light chain Antibody Sequence

TABLE 12
Nectin-4 antibody mouse IgG2a light chain

| Antibody | Sequence |
|---|---|
| TNT-188 | divmtqspsslsasvgdrvtitcrasqsissylnwyqqkpgkapklliyaasslqsgvpsrfsgsgsgtdftltisslqpedfa |
| | tyycqqsystpltfgqgtkveiKRadaaptvsifppsseqltsggasvvcflnnfypkdinvkwkidgserqngvlnswtdqd |
| | skdstysmsstltltkdeyerhnsytceathktstspivksfnrnec (SEQ ID NO: 212) |
| TNT-189 | diqltqspsslsasvgdrvtitcrasqgisnylawyqqkpgkapklliyaastlqsgvpsrfsgsgsgtdftltisslqpedfa |
| | tyycqqsystpytfgqgtkleiKRadaaptvsifppsseqltsggasvvcflnnfypkdinvkwkidgserqngvlnswtdqds |
| | kdstysmsstltltkdeyerhnsytceathktstspivksfnrnec (SEQ ID NO: 213) |
| TNT-190 | eivltqspatlslspgeratlscrasqsvssylawyqqkpgqaprlliydasnratgiparfsgsgsgtdftltisslqaedfa |
| | tyycqqsystrfgggtkveiKRadaaptvsifppsseqltsggasvvcflnnfypkdinvkwkidgserqngvlnswtdqdskd |
| | stysmsstltltkdeyerhnsytceathktstspivksfnrnec (SEQ ID NO: 214) |
| TNT-191 | eivltqspatlslspgeratlscrasqsvsssylawyqqkpgqaprlliygassratgipdrfsgsgsgtdftltisrlepedf |
| | avyycqqynnwwtfgqgtkvdiKRadaaptvsifppsseqltsggasvvcflnnfypkdinvkwkidgserqngvlnswtdqd |
| | skdstysmsstltltkdeyerhnsytceathktstspivksfnrnec (SEQ ID NO: 215) |
| TNT-192 | divmtqspsslsasvgdrvtitcrasqgisnylawyqqkpgkvpklliyaastlqsgvpsrfsgsgsgtdftltisslqpedva |
| | tyycqkynsapytfgqgtkveiKRadaaptvsifppsseqltsggasvvcflnnfypkdinvkwkidgserqngvlnswtd |
| | qdskdstysmsstltltkdeyerhnsytceathktstspivksfnrnec (SEQ ID NO: 216) |
| TNT-193 | eivltqspatlsvspgeratlscrasqsvssnlawyqqkpgqaprlliygassratgipdrfsgsgsgtdftltisrlepedfa |
| | vyychqygsspltfgggtkleiKRadaaptvsifppsseqltsggasvvcflnnfypkdinvkwkidgserqngvlnswtdqds |
| | kdstysmsstltltkdeyerhnsytceathktstspivksfnrnec (SEQ ID NO: 217) |
| TNT-194 | diqmtqspsslsasvgdrvtitcrasqgisnylawfqqkpgkapkslisaasslqsgvpsrfsgsgsgtdftltisslqpedf |
| | atyyclqhdsfpitfgggtkleiKRadaaptvsifppsseqltsggasvvcflnnfypkdinvkwkidgserqngvlnswtdq |
| | dskdstysmsstltltkdeyerhnsytceathktstspivksfnrnec (SEQ ID NO: 218) |
| TNT-195 | diqmtqspstlsasvgdrvtitcrasqgisnylawyqqkpgkapklliyaasslqsgvpsrfsgsgsgtdftltisslqpedfa |
| | tyycqqsystpltfgggtkveiKRadaaptvsifppsseqltsggasvvcflnnfypkdinvkwkidgserqngvlnswtdqd |
| | skdstysmsstltltkdeyerhnsytceathktstspivksfnrnec (SEQ ID NO: 219) |
| TNT-196 | diqmtqspsslsasvgdrvtitcqasqdisnylnwyqqkpgkvpklliyaastlqsgvpsrfsgsgsgtdftltisslqpedfa |
| | tyycqqsystpltfgggtkveiKRadaaptvsifppsseqltsggasvvcflnnfypkdinvkwkidgserqngvlnswtdq |
| | dskdstysmsstltltkdeyerhnsytceathktstspivksfnrnec (SEQ ID NO: 220) |
| TNT-197 | eivltqspgtlslspgeratlscrasqsvsssylawyqqkpgqaprlliygassratgipdrfsgsgsgtdftltisrlepedf |
| | avyycqqygssltfgggtkveiKRadaaptvsifppsseqltsggasvvcflnnfypkdinvkwkidgserqngvlnswtdqds |
| | kdstysmsstltltkdeyerhnsytceathktstspivksfnrnec (SEQ ID NO: 221) |
| TNT-198 | divmtqspsslsasvgdrvtitcrasqgisnylawyqqkpgkvpklliyaastlqsgvpsrfsgsgsgteftlaisslqpedla |
| | tyycqqlnshpitfgqgtrleiKRadaaptvsifppsseqltsggasvvcflnnfypkdinvkwkidgserqngvlnswtdqds |
| | kdstysmsstltltkdeyerhnsytceathktstspivksfnrnec (SEQ ID NO: 222) |
| TNT-153 | Diqmtqspssvsasvgdrvtitcrasqgisgwlawyqqkpgkapkfliyaastlqsgvpsrfsgsgsgtdftltisslqpedfa |
| | tyycqqansfpptfgggtkveikradaaptvsifppsseqltsggasvvcflnnfypkdinvkwkidgserqngvlnswtdq |
| | dskdstysmsstltltkdeyerhnsytceathktstspivksfnrnec (SEQ ID NO: 223) |

TABLE 13
Nectin-4 antibody mouse IgG2a heavy chain with Q tag

| Antibody | Sequence |
|---|---|
| TNT-188 | evqlvqsgaevkkpgssvkvsckasgytftgyymhwvrqapgqglewmgwinpnsggtnyaqkfqgrvtmtrdtsist |
| | aymelrslrsddtavyycargsalhffgvfdywgqgtlvtvsSakttapsvyplapvcgdttgssvtlgclvkgyfpepvtlt |
| | wnsgslssgvhtfpavlqsdlytlsssvtvtsstwpsqsitcnvahpasstkvdkkieprgptikpcppckcpapnllggpsv |
| | fifppkikdvlmislspivtcvvvdvseddpdvqiswfvnnvevhtaqtqthredynstlrvvsalpiqhqdwmsgkefkc |
| | kvnnkdlpapiertiskpkgsvrapqvyvlpppeeemtkkqvtltcmvtdfmpediyvewtnngktelnykntepvldsd |
| | gsyfmysklrvekknwvernsyscsvvheglhnhhttksfsrtpgrpqgfgpp (SEQ ID NO: 200) |
| TNT-189 | evqlvqsgaevkkpgssvkvsckasggtfssyaiswvrqapgqglewmggiipifgtanyaqkfqgrvtitadeststaym |
| | elsslrsedtavyycargyssswyepyyfdywgqgtlvtvsSakttapsvyplapvcgdttgssvtlgclvkgyfpepvtltw |
| | nsgslssgvhtfpavlqsdlytlsssvtvtsstwpsqsitenvahpasstkvdkkieprgptikpcppckcpapnllggpsvf |
| | ifppkikdvlmislspivtcvvvdvseddpdvqiswfvnnvevhtaqtqthredynstlrvvsalpighqdwmsgkefkckv |
| | nnkdlpapiertiskpkgsvrapqvyvlpppeeemtkkqvtltcmvtdfmpediyvewtnngktelnykntepvldsdgs |
| | yfmysklrvekknwvernsyscsvvheglhnhhttksfsrtpgrpqgfgpp (SEQ ID NO: 201) |
| TNT-190 | evqlvqsggglvkpggslrlscaasgftfsdyymswvrqapgkglewvsaisgsggstyyadsvkgrftisrdnskntlylq |
| | mnslraedtavyycardegyyymdvwgkgttvtvsSakttapsvyplapvcgdttgssvtlgclvkgyfpepvtltwnsgs |
| | lssgvhtfpavlqsdlytlsssvtvtsstwpsqsitcnvahpasstkvdkkieprgptikpcppckcpapnllggpsvfifpp |
| | kikdvlmislspivtcvvvdvseddpdvqiswfvnnvevhtaqtqthredynstlrvvsalpiqhqdwmsgkefkckvnnk |
| | dlpapiertiskpkgsvrapqvyvlpppeeemtkkqvtltcmvtdfmpediyvewtnngktelnykntepvldsdgsyfm |
| | ysklrvekknwvernsyscsvvheglhnhhttksfsrtpgrpqgfgpp (SEQ ID NO: 202) |
| TNT-191 | evqllesgggvvqpgrslrlscaasgftfssygmhwvrqapgkglewvavisydgsnkyyadsvkgrftisrdnskntlylq |
| | mnslraedtavyycakssyydyvwgsyrytdafdiwgqgtmvtvsSakttapsvyplapvcgdttgssvtlgclvkgyfp |
| | epvtltwnsgslssgvhtfpavlqsdlytlsssvtvtsstwpsqsitenvahpasstkvdkkieprgptikpcppckcpapnl |
| | lggpsvfifppkikdvlmislspivtcvvvdvseddpdvqiswfvnnvevhtaqtqthredynstlrvvsalpiqhqdwmsg |
| | kefkckvnnkdlpapiertiskpkgsvrapqvyvlpppeeemtkkqvtltcmvtdfmpediyvewtnngktelnykntep |
| | vldsdgsyfmysklrvekknwvernsyscsvvheglhnhhttksfsrtpgrpqgfgpp (SEQ ID NO: 203) |
| TNT-192 | evqllesgggvvqpgrslrlscaasgftfssygmhwvrqapgkglewvavisydgsnkyyadsvkgrftisrdnskntlylq |
| | mnslraedtavyycakdygynwnffdywgqgtlvtvsSakttapsvyplapvcgdttgssvtlgclvkgyfpepvtltwns |
| | gslssgvhtfpavlqsdlytlsssvtvtsstwpsqsitcnvahpasstkvdkkieprgptikpcppckcpapnllggpsvfif |
| | ppkikdvlmislspivtcvvvdvseddpdvqiswfvnnvevhtaqtqthredynstlrvvsalpiqhqdwmsgkefkckvnn |
| | kdlpapiertiskpkgsvrapqvyvlpppeeemtkkqvtltcmvtdfmpediyvewtnngktelnykntepvldsdgsyf |
| | mysklrvekknwvernsyscsvvheglhnhhttksfsrtpgrpqgfgpp (SEQ ID NO: 204) |
| TNT-193 | qvqlqesgpglvkpsgtlsltctvsggsissggyywawirqppgkglewigsiyytgstyynpslksrvtiyvdtsknqfslk |
| | lssvtaadtavyycardpfpeppdafdiwgqgtmvtvsSakttapsvyplapvcgdttgssvtlgclvkgyfpepvtltwnsg |
| | slssgvhtfpavlqsdlytlsssvtvtsstwpsqsitenvahpasstkvdkkieprgptikpcppckcpapnllggpsvfifp |
| | pkikdvlmislspivtcvvvdvseddpdvqiswfvnnvevhtaqtqthredynstlrvvsalpiqhqdwmsgkefkckvnnk |
| | dlpapiertiskpkgsvrapqvyvlpppeeemtkkqvtltcmvtdfmpediyvewtnngktelnykntepvldsdgsyfm |
| | ysklrvekknwvernsyscsvvheglhnhhttksfsrtpgrpqgfgpp (SEQ ID NO: 205) |
| TNT-194 | evqlvqsgaevkkpgasvkvsckasgytftsydinwvrqapgqglewmggiipifgtanyaqkfqgrvtitadeststaym |
| | elrslrsedtavyycastavagfgidywgqgtlvtvsSakttapsvyplapvcgdttgssvtlgclvkgyfpepvtltwnsgs |
| | lssgvhtfpavlqsdlytlsssvtvtsstwpsqsitcnvahpasstkvdkkieprgptikpcppckcpapnllggpsvfifpp |
| | kikdvlmislspivtcvvvdvseddpdvqiswfvnnvevhtaqtqthredynstlrvvsalpiqhqdwmsgkefkckvnnkdl |
| | papiertiskpkgsvrapqvyvlpppeeemtkkqvtltcmvtdfmpediyvewtnngktelnykntepvldsdgsyfmy |
| | sklrvekknwvernsyscsvvheglhnhhttksfsrtpgrpqgfgpp (SEQ ID NO: 206) |
| TNT-195 | evqlvqsgaevkkpgssvkvsckasggtfssyaiswvrqapgqglewmggiipifgtanyaqkfqgrvtitadkststaym |
| | elsslrsedtavyycaagyyydssgyyhqpyyfdywgqgtlvtvsSakttapsvyplapvcgdttgssvtlgclvkgyfpep |
| | vtltwnsgslssgvhtfpavlqsdlytlsssvtvtsstwpsqsitcnvahpasstkvdkkieprgptikpcppckcpapnllg |
| | gpsvfifppkikdvlmislspivtcvvvdvseddpdvqiswfvnnvevhtaqtqthredynstlrvvsalpiqhqdwmsgke |
| | fkckvnnkdlpapiertiskpkgsvrapqvyvlpppeeemtkkqvtltcmvtdfmpediyvewtnngktelnykntepvl |
| | dsdgsyfmysklrvekknwvernsyscsvvheglhnhhttksfsrtpgrpqgfgpp (SEQ ID NO: 207) |
| TNT-196 | evqlvesggglvqpggslrlscaasgftfsdyymswirqapgkglewvsgiswnsgsigyadsvkgrftisrdnskntlylq |
| | mnslraedtavyycassgswylpldywgqgtlvtvsSakttapsvyplapvcgdttgssvtlgclvkgyfpepvtltwnsgs |
| | lssgvhtfpavlqsdlytlsssvtvtsstwpsqsitcnvahpasstkvdkkieprgptikpcppckcpapnllggpsvfifpp |
| | kikdvlmislspivtcvvvdvseddpdvqiswfvnnvevhtaqtqthredynstlrvvsalpiqhqdwmsgkefkckvnnk |
| | dlpapiertiskpkgsvrapqvyvlpppeeemtkkqvtltcmvtdfmpediyvewtnngktelnykntepvldsdgsyfm |
| | ysklrvekknwvernsyscsvvheglhnhhttksfsrtpgrpqgfgpp (SEQ ID NO: 208) |
| TNT-197 | evqlvqsgggvvqpgrslrlscaasgftfssyamhwvrqapgkglewvavisydgsnkyyadsvkgrftisrdnskntlyl |
| | qmnslraedtavyycaratlqlwlplfdywgqgtlvtvsSakttapsvyplapvcgdttgssvtlgclvkgyfpepvtltwns |
| | gslssgvhtfpavlqsdlytlsssvtvtsstwpsqsitcnvahpasstkvdkkieprgptikpcppckcpapnllggpsvfif |
| | ppkikdvlmislspivtcvvvdvseddpdvqiswfvnnvevhtaqtqthredynstlrvvsalpiqhqdwmsgkefkckvnn |
| | kdlpapiertiskpkgsvrapqvyvlpppeeemtkkqvtltcmvtdfmpediyvewtnngktelnykntepvldsdgsyf |
| | mysklrvekknwvernsyscsvvheglhnhhttksfsrtpgrpqgfgpp (SEQ ID NO: 209) |
| TNT-198 | evqlvqsgaevkkpgssvkvsckasggtfssyaiswvrqapgqglewmgwinpnsggtnyaqkfqgrvtmtrdtststa |
| | ymelsslrsedtavyycatgyygdlyyfdywgqgtlvtvsSakttapsvyplapvcgdttgssvtlgclvkgyfpepvtltw |
| | nsgslssgvhtfpavlqsdlytlsssvtvtsstwpsqsitenvahpasstkvdkkieprgptikpcppckcpapnllggpsvf |
| | ifppkikdvlmislspivtcvvvdvseddpdvqiswfvnnvevhtaqtqthredynstlrvvsalpiqhqdwmsgkefkckv |
| | nnkdlpapiertiskpkgsvrapqvyvlpppeeemtkkqvtltcmvtdfmpediyvewtnngktelnykntepvldsdgs |
| | yfmysklrvekknwvernsyscsvvheglhnhhttksfsrtpgrpqgfgpp (SEQ ID NO: 210) |
| TNT-153 | Evqlvesggglvqpggslrlscaasgftfssynmnwvrqapgkglewvsyissssstiyyadsvkgrftisrdnaknslslq |
| | mnslrdedtavyycarayyygmdvwgqgttvtvssakttapsvyplapvcgdttgssvtlgclvkgyfpepvtltwnsgsl |
| | ssgvhtfpavlqsdlytlsssvtvtsstwpsqsitcnvahpasstkvdkkieprgptikpcppckcpapnllggpsvfifppk |
| | ikdvlmislspivtcvvvdvseddpdvqiswfvnnvevhtaqtqthredynstlrvvsalpiqhqdwmsgkefkckvnnk |
| | dlpapiertiskpkgsvrapqvyvlpppeeemtkkqvtltcmvtdfmpediyvewtnngktelnykntepvldsdgsyfm |
| | ysklrvekknwvernsyscsvvheglhnhhttksfsrtpgllqgg (SEQ ID NO: 211) |

In some embodiments, the nectin-4 antibody or conjugate comprises a light chain comprising a sequence selected from the group consisting of the sequences disclosed in Table 14, below. In some embodiments, the nectin-4 antibody or conjugate comprises a heavy chain comprising a Q tag comprising a sequence selected from the group consisting of the sequences disclosed in Table 15, below. In some embodiments, the nectin-4 antibody or conjugate comprises a heavy chain comprising a sequence selected from the group consisting of the sequences disclosed in Table 15A, below. In some embodiments, the nectin-4 antibody or conjugate comprises a light chain comprising a sequence selected from the group consisting of the sequences disclosed in Table 14 and a heavy chain comprising a Q tag comprising a sequence selected from the group consisting of the sequences disclosed in Table 15. In some embodiments, the nectin-4 antibody or conjugate comprises a light chain comprising a sequence selected from the group consisting of the sequences disclosed in Table 14 and a heavy chain comprising a sequence selected from the group consisting of the sequences disclosed in Table 15A.

TABLE 14
Nectin-4 antibody human IgG1 light chain

| Antibody | Sequence |
|---|---|
| TNT-201; | divmtqspsslsasvgdrvtitcrasqsissylnwyqqkpgkapklliyaasslqsgvpsrfsgsgsgtdftltisslqpedfa |
| TNT-413 | tyycqqsystpltfgqgtkveiKRtvaapsvfifppsdeqlksgtasvvcllnnfypreakvqwkvdnalqsgnsqesvteqd |
| | skdstyslsstltlskadyekhkvyacevthqglsspvtksfnrgec (SEQ ID NO: 236) |
| TNT-202 | diqltqspsslsasvgdrvtitcrasqgisnylawyqqkpgkapklliyaastlqsgvpsrfsgsgsgtdftltisslqpedfa |
| | tyycqqsystpytfgqgtkleiKRtvaapsvfifppsdeqlksgtasvvcllnnfypreakvqwkvdnalqsgnsqesvteqds |
| | kdstyslsstltlskadyekhkvyacevthqglsspvtksfnrgec (SEQ ID NO: 237) |
| TNT-203 | eivltqspatlslspgeratlscrasqsvssylawyqqkpgqaprlliydasnratgiparfsgsgsgtdftltisslqaedfa |
| | tyycqqsystrfgggtkveiKRtvaapsvfifppsdeqlksgtasvvcllnnfypreakvqwkvdnalqsgnsqesvteqdskd |
| | styslsstltlskadyekhkvyacevthqglsspvtksfnrgec (SEQ ID NO: 238) |
| TNT-204 | eivltqspatlslspgeratlscrasqsvsssylawyqqkpgqaprlliygassratgipdrfsgsgsgtdftltisrlepedf |
| | avyycqqynnwwtfgqgtkvdiKRtvaapsvfifppsdeqlksgtasvvcllnnfypreakvqwkvdnalqsgnsqesvteqd |
| | skdstyslsstltlskadyekhkvyacevthqglsspvtksfnrgec (SEQ ID NO: 239) |
| TNT-205 | divmtqspsslsasvgdrvtitcrasqgisnylawyqqkpgkvpklliyaastlqsgvpsrfsgsgsgtdftltisslqpedva |
| | tyycqkynsapytfgqgtkveiKRtvaapsvfifppsdeqlksgtasvvcllnnfypreakvqwkvdnalqsgnsqesvte |
| | qdskdstyslsstltlskadyekhkvyacevthqglsspvtksfnrgec (SEQ ID NO: 240) |
| TNT-206 | eivltqspatlsvspgeratlscrasqsvssnlawyqqkpgqaprlliygassratgipdrfsgsgsgtdftltisrlepedfa |
| | vyychqygsspltfgggtkleiKRtvaapsvfifppsdeqlksgtasvvcllnnfypreakvqwkvdnalqsgnsqesvteqds |
| | kdstyslsstltlskadyekhkvyacevthqglsspvtksfnrgec (SEQ ID NO: 241) |
| TNT-207; | diqmtqspsslsasvgdrvtitcrasqgisnylawfqqkpgkapkslisaasslqsgvpsrfsgsgsgtdftltisslqpedfa |
| TNT-422 | tyyclqhdsfpitfgggtkleiKRtvaapsvfifppsdeqlksgtasvvcllnnfypreakvqwkvdnalqsgnsqesvteqds |
| | kdstyslsstltlskadyekhkvyacevthqglsspvtksfnrgec (SEQ ID NO: 242) |
| TNT-208; | diqmtqspstlsasvgdrvtitcrasqgisnylawyqqkpgkapklliyaasslqsgvpsrfsgsgsgtdftltisslqpedfa |
| TNT-423 | tyycqqsystpltfgggtkveiKRtvaapsvfifppsdeqlksgtasvvcllnnfypreakvqwkvdnalqsgnsqesvteqd |
| | skdstyslsstltlskadyekhkvyacevthqglsspvtksfnrgec (SEQ ID NO: 243) |
| TNT-209 | diqmtqspsslsasvgdrvtitcqasqdisnylnwyqqkpgkvpklliyaastlqsgvpsrfsgsgsgtdftltisslqpedfa |
| | tyycqqsystpltfgggtkveiKRtvaapsvfifppsdeqlksgtasvvcllnnfypreakvqwkvdnalqsgnsqesvteq |
| | dskdstyslsstltlskadyekhkvyacevthqglsspvtksfnrgec (SEQ ID NO: 244) |
| TNT-210; | eivltqspgtlslspgeratlscrasqsvsssylawyqqkpgqaprlliygassratgipdrfsgsgsgtdftltisrlepedf |
| TNT-424 | avyycqqygssltfgggtkveiKRtvaapsvfifppsdeqlksgtasvvcllnnfypreakvqwkvdnalqsgnsqesvteqds |
| | kdstyslsstltlskadyekhkvyacevthqglsspvtksfnrgec (SEQ ID NO: 245) |
| TNT-211 | divmtqspsslsasvgdrvtitcrasqgisnylawyqqkpgkvpklliyaastlqsgvpsrfsgsgsgteftlaisslqpedla |
| | tyycqqlnshpitfgqgtrleiKRtvaapsvfifppsdeqlksgtasvvcllnnfypreakvqwkvdnalqsgnsqesvteqds |
| | kdstyslsstltlskadyekhkvyacevthqglsspvtksfnrgec (SEQ ID NO: 246) |
| TNT-154; | diqmtqspssvsasvgdrvtitcrasqgisgwlawyqqkpgkapkfliyaastlqsgvpsrfsgsgsgtdftltisslqpedfa |
| TNT-212 | tyycqqansfpptfgggtkveiKRtvaapsvfifppsdeqlksgtasvvcllnnfypreakvqwkvdnalqsgnsqesvteq |
| | dskdstyslsstltlskadyekhkvyacevthqglsspvtksfnrgec (SEQ ID NO: 247) |
| TNT-317 | QSVLTQPPSVSAAPGQKVTISCSGSSSNIGNNYVSWYQQFPGTAPKLLIYDNNKRP |
| | SGIPDRFSGSKSGTSATLGITGLQTGDEADYYCGTWDSSLNAGWVFGGGTKLTV |
| | Lgqpkanptvtlfppsseelqankatlvclisdfypgavtvawkadgspvkagvettkpskqsnnkyaassylsltpeqwk |
| | shrsyscqvthegstvektvaptecs |
| | (SEQ ID NO: 1000) |
| TNT-318 | QTVLTQPSSLSASPGASASLTCTLRSGINVGSYRIYWYQQKPGSPPQFLLRYKSDS |
| | DKQQGSGVPSRFSGSKDDSANAGILLISGLQSEDEADYYCMIWHSSASVFGTGTK |
| | VTVLgqpkanptvtlfppsseelqankatlvclisdfypgavtvawkadgspvkagvettkpskqsnnkyaassylsltp |
| | eqwkshrsyscqvthegstvektvaptecs (SEQ ID NO: 1001) |
| TNT-320 | QSVLTQSPSASGTPGQRVTISCSGSSSNIGSNTVHWYQQLPGTAPKLLMYGNYQR |
| | PSGVPARFSGSKSGTSASLAISGLQSEDEADYYCATWDDSLNGWVFGGGTKVTV |
| | Lgqpkanptvtlfppsseelqankatlvclisdfypgavtvawkadgspvkagvettkpskqsnnkyaassylsltpeqwk |
| | shrsyscqvthegstvektvaptecs (SEQ ID NO: 1002) |
| TNT-321 | DIQMTQSPSAMSASVGDRVTITCRASQGISNYLAWFQQKPGKVPKRLIYAASSLQ |
| | SGVPSRFSGSGSGTEFTLTISSLQPEDFATYYCLQHSFYPLTFGPGTKVDIKRtvaaps |
| | vfifppsdeqlksgtasvvclInnfypreakvqwkvdnalqsgnsqesvteqdskdstyslsstltlskadyekhkvyacevt |
| | hqglsspvtksfnrgec (SEQ ID NO: 1003) |
| TNT-322 | QSVLTQPPSVSAAPGQKVTISCSGSSSNIGNNYVSWYQQFPGTAPKLLIYDNNKRP |
| | SGIPDRFSGSKSGTSATLGITGLQTGDEADYYCGTWDSSLSAGVVFGGGTKLTVL |
| | gqpkanptvtlfppsseelqankatlvclisdfypgavtvawkadgspvkagvettkpskqsnnkyaassylsltpeqwks |
| | hrsyscqvthegstvektvaptecs (SEQ ID NO: 1004) |
| TNT-323 | DVVMTQSPLSLPVTLGQPASMSCRSSQSLAYSDGNTYLNWFQQRPGQSPRRLIY |
| | KVSNRDSGVPDRFSGSGSGTDFTLKISRVEAEDVGVYYCMQGTHWPLTFGGGTK |
| | VEIKRtvaapsvfifppsdeqlksgtasvvcllnnfypreakvqwkvdnalqsgnsqesvteqdskdstyslsstltlskad |
| | yekhkvyacevthqglsspvtksfnrgec (SEQ ID NO: 1005) |
| TNT-324 | QTVVTQEPSLTVSPGGTVTLTCASSTGAVTSGYYPNWFQQKPGQAPRALIYSTSN |
| | KHSWTPARFSGSLLGGKAALTLSGVQPEDEAEYYCLLYYDGAHLFGGGTKLTVL |
| | gqpkanptvtlfppsseelqankatlvclisdfypgavtvawkadgspvkagvettkpskqsnnkyaassylsltpeqwks |
| | hrsyscqvthegstvektvaptecs (SEQ ID NO: 1006) |
| TNT-327; | QSALTQPASVSGSPGQSITISCTGTTSDIGSYNLVSWYQQHPGKAPKLMIYEVSMS |
| TNT-425 | PSGVSDRFSGSKSGSTASLTISGLQAEDEADYYCSSYAGSSTVVFGGGTRLTVLgq |
| | pkanptvtlfppsseelqankatlvclisdfypgavtvawkadgspvkagvettkpskqsnnkyaassylsltpeqwkshrs |
| | yscqvthegstvektvaptecs |
| | (SEQ ID NO: 1007) |
| TNT-328; | QSVLTQPPSVSGAPGQRVTISCTGSSSNIGAGYNVHWYQQLPGTAPKVLISGNSN |
| TNT-426 | RPSGVPDRFSGSKSGTSASLAITGLQAEDEADYYCQSYDSSLSGSAVFGGGTKLT |
| | VLgqpkanptvtlfppsseelqankatlvclisdfypgavtvawkadgspvkagvettkpskqsnnkyaassylsltpeqw |
| | kshrsyscqvthegstvektvaptecs (SEQ ID NO: 1008) |
| TNT-330 | DIVMTQTPLSLPVTPGEPASISCRSSQSLLDSDDGNTYLDWYLQKPGQSPQLLIYT |
| | LSYRASGVPDRFSGSGSGTDFTLKVSRVEAEDVGVYYCMQRIEFPYTFGQGTKLE |
| | IKRtvaapsvfifppsdeqlksgtasvvcllnnfypreakvqwkvdnalqsgnsqesvteqdskdstyslsstltlskadyek |
| | hkvyacevthqglsspvtksfnrgec (SEQ ID NO: 1009) |
| TNT-331 | QSVLTQPPSVSAAPGQKVTISCSGSSSNIGNNYVSWYQQLPGTAPKLLIYDNNKR |
| | PSGIPDRFSGSKSGTSATLGITGLQTGDEADYYCGTWDTSLSSGWLFGGGTKLTV |
| | Lgqpkanptvtlfppsseelqankatlvclisdfypgavtvawkadgspvkagvettkpskqsnnkyaassylsltpeqwk |
| | shrsyscqvthegstvektvaptecs (SEQ ID NO: 1010) |
| TNT-332 | DIQLTQSPSFLSASVGDKVTITCRASQGINSYLAWYQQKPGKAPKLLIYSASTLQG |
| | GVPSRFSGSGSGTEFTLTISNLQPADFATYCCQQLNSYPFTFGPGTKVDIKRtvaapsv |
| | fifppsdeqlksgtasvvcllnnfypreakvqwkvdnalqsgnsqesvteqdskdstyslsstltlskadyekhkvyacevth |
| | qglsspvtksfnrgec (SEQ ID NO: 1011) |
| TNT-333; | QSVLTQPPSVSAAPGQKVTISCSGSSSNIGNNYVSWYQQLPGTAPKLLISDNNKRP |
| TNT-427 | SGIPDRFSGSKSGTSATLGITGLQTGDEADYYCGTWDSSLSAGVVFGGGTKLTVL |
| | gqpkanptvtlfppsseelqankatlvclisdfypgavtvawkadgspvkagvettkpskqsnnkyaassylsltpeqwks |
| | hrsyscqvthegstvektvaptecs (SEQ ID NO: 1012) |
| TNT-334 | EIVMTQSPATLPVSPGERATLSCRASQSVSINLAWYQQKPGQAPRLLIYGVSTRAT |
| | GIPARFSGSGSGTEFTLTISGLQSEDFAVYYCQQYNNWPRTFGQGTKVEIKRtvaaps |
| | vfifppsdeqlksgtasvvcllnnfypreakvqwkvdnalqsgnsqesvteqdskdstyslsstltlskadyekhkvyacevt |
| | hqglsspvtksfnrgec (SEQ ID NO: 1013) |
| TNT-335 | QSVLTQPPSASGTPGQRVTISCSGSSSNIGSDYIYWYQQLPGTAPKLLIYSNHQRPS |
| | GVPDRFSGSKSGTSASLAISGLRSEDEADYYCAAWEDSLSGPIFGGGTKLTVLgqp |
| | kanptvtlfppsseelqankatlvclisdfypgavtvawkadgspvkagvettkpskqsnnkyaassylsltpeqwkshrsy |
| | scqvthegstvektvaptecs (SEQ ID NO: 1014) |
| TNT-336 | QLVLTQSPSASASLGASVKLTCTLSSGHSFYAIAWHQQQPEKGPRYLMQVNSNG |
| | SHNKGDGIPDRFSGSSSGAERYLTISSLQSEDEADYYCQSWDTDIWVFGGGTKLT |
| | VLgqpkanptvtlfppsseelqankatlvclisdfypgavtvawkadgspvkagvettkpskqsnnkyaassylsltpeqw |
| | kshrsyscqvthegstvektvaptecs (SEQ ID NO: 1015) |
| TNT-337 | DIVMTQTPLSLPVTPGEPASISCRSSQSIVHSNANTYLEWYLQKPGQSPQLLIYKVS |
| | NRFSGVPDRFSGSGSGTDFTLKISRVEAEDVGVYYCFQGSHVPYTFGGGTKVEIK |
| | Rtvaapsvfifppsdeqlksgtasvvcllnnfypreakvqwkvdnalqsgnsqesvteqdskdstyslsstltlskadyekhk |
| | vyacevthqglsspvtksfnrgec (SEQ ID NO: 1016) |
| TNT-412; | DIQMTQSPSSLSASVGDRVTITCRASQSISSYLNWYQQKPGKAPKLLIYAASSLqS |
| TNT-414 | GVPSRFSGSGSGTDFTLTISSLQPEDFATYYCQQSYSTPLTFGQGTKVEIKRtvaapsv |
| | fifppsdeqlksgtasvvcllnnfypreakvqwkvdnalqsgnsqesvteqdskdstyslsstltlskadyekhkvyacevth |
| | qglsspvtksfnrgec (SEQ ID NO: 1017) |
| TNT-417; | DIQMTQSPSSLSASVGDRVTITCRASQGISNYLAWYQQKPGKAPKLLIYAASSLqS |
| TNT-428 | GVPSRFSGSGSGTDFTLTISSLQPEDFATYYCQQSYSTPLTFGGGTKVEIKRtvaapsv |
| | fifppsdeqlksgtasvvcllnnfypreakvqwkvdnalqsgnsqesvteqdskdstyslsstltlskadyekhkvyacevth |
| | qglsspvtksfnrgec (SEQ ID NO: 1018) |
| TNT-418; | QSALTQPASVSGSPGQSITISCTGTTSDIGSYNLVSWYQQHPGKAPKLMIYEVSMS |
| TNT-429 | PSGVSNRFSGSKSGNTASLTISGLQAEDEADYYCSSYAGSSTVVFGGGTRLTVLgq |
| | pkanptvtlfppsseelqankatlvclisdfypgavtvawkadgspvkagvettkpskqsnnkyaassylsltpeqwkshrs |
| | yscqvthegstvektvaptecs (SEQ ID NO: 1019) |
| TNT-419; | QSALTQPASVSGSPGQSITISCTGTTSDIGSYNLVSWYQQHPGKAPKLMIYEVSMS |
| TNT-430 | PSGVSNRFSGSKSGNTASLTISGLQAEDEADYYCSSYAGSSTVVFGGGTRLTVLgq |
| | pkanptvtlfppsseelqankatlvclisdfypgavtvawkadgspvkagvettkpskqsnnkyaassylsltpeqwkshrs |
| | yscqvthegstvektvaptecs (SEQ ID NO: 1020) |
| TNT-420; | QSVLTQPPSVSGAPGQRVTISCTGSSSNIGAGYNVHWYQQLPGTAPKLLIYGNSN |
| TNT-431 | RPSGVPDRFSGSKSGTSASLAITGLQAEDEADYYCQSYDSSLSGSAVFGGGTKLT |
| | VLgqpkanptvtlfppsseelqankatlvclisdfypgavtvawkadgspvkagvettkpskqsnnkyaassylsltpeqw |
| | kshrsyscqvthegstvektvaptecs (SEQ ID NO: 1021) |
| TNT-421; | QSVLTQPPSVSAAPGQKVTISCSGSSSNIGNNYVSWYQQLPGTAPKLLIYDNNKR |
| TNT-432 | PSGIPDRFSGSKSGTSATLGITGLQTGDEADYYCGTWDSSLSAGVVFGGGTKLTV |
| | Lgqpkanptvtlfppsseelqankatlvclisdfypgavtvawkadgspvkagvettkpskqsnnkyaassylsltpeqwk |
| | shrsyscqvthegstvektvaptecs (SEQ ID NO: 1022) |

TABLE 15
Nectin-4 antibody human IgG1 heavy chain with a Q-tag

| Antibody | Sequence |
|---|---|
| TNT-201 | evqlvqsgaevkkpgssvkvsckasgytftgyymhwvrqapgqglewmgwinpnsggtnyaqkfqgrvtmtrdtsist |
| with Q tag | aymelrslrsddtavyycargsalhffgvfdywgqgtlvtvsSastkgpsvfplapsskstsggtaalgclvkdyfpepvtvs |
| | wnsgaltsgvhtfpavlqssglyslssvvtvpssslgtqtyicnvnhkpsntkvdkkvepkscdkthtcppcpapellggpsv |
| | flfppkpkdtlmisrtpevtcvvvdvshedpevkfnwyvdgvevhnaktkpreeqynstyrvvsvltvlhqdwlngkey |
| | kckvsnkalpapiektiskakgqprepqvytlppsreemtknqvsltclvkgfypsdiavewesngqpennykttppvlds |
| | dgsfflyskltvdksrwqqgnvfscsvmhealhnhytqkslslspgrpqgfgpp (SEQ ID NO: 224) |
| TNT-202 | evqlvqsgaevkkpgssvkvsckasggtfssyaiswvrqapgqglewmggiipifgtanyaqkfqgrvtitadeststaym |
| with Q tag | elsslrsedtavyycargyssswyepyyfdywgqgtlvtvsSastkgpsvfplapsskstsggtaalgclvkdyfpepvtvs |
| | wnsgaltsgvhtfpavlqssglyslssvvtvpssslgtqtyicnvnhkpsntkvdkkvepkscdkthtcppcpapellggpsv |
| | flfppkpkdtlmisrtpevtcvvvdvshedpevkfnwyvdgvevhnaktkpreeqynstyrvvsvltvlhqdwlngkey |
| | kckvsnkalpapiektiskakgqprepqvytlppsreemtknqvsltclvkgfypsdiavewesngqpennykttppvlds |
| | dgsfflyskltvdksrwqqgnvfscsvmhealhnhytqkslslspgrpqgfgpp (SEQ ID NO: 225) |
| TNT-203 | evqlvqsggglvkpggslrlscaasgftfsdyymswvrqapgkglewvsaisgsggstyyadsvkgrftisrdnskntlylq |
| with Q tag | mnslraedtavyycardegyyymdvwgkgttvtvsSastkgpsvfplapsskstsggtaalgclvkdyfpepvtvswnsg |
| | altsgvhtfpavlqssglyslssvvtvpssslgtqtyicnvnhkpsntkvdkkvepkscdkthtcppcpapellggpsvflfpp |
| | kpkdtlmisrtpevtcvvvdvshedpevkfnwyvdgvevhnaktkpreeqynstyrvvsvltvlhqdwlngkeykckvs |
| | nkalpapiektiskakgqprepqvytlppsreemtknqvsltclvkgfypsdiavewesngqpennykttppvldsdgsffl |
| | yskltvdksrwqqgnvfscsvmhealhnhytqkslslspgrpqgfgpp (SEQ ID NO: 226) |
| TNT-204 | evqllesgggvvqpgrslrlscaasgftfssygmhwvrqapgkglewvavisydgsnkyyadsvkgrftisrdnskntlylq |
| with Q tag | mnslraedtavyycakssyydyvwgsyrytdafdiwgqgtmvtvsSastkgpsvfplapsskstsggtaalgclvkdyfp |
| | epvtvswnsgaltsgvhtfpavlqssglyslssvvtvpssslgtqtyicnvnhkpsntkvdkkvepkscdkthtcppcpapel |
| | lggpsvflfppkpkdtlmisrtpevtcvvvdvshedpevkfnwyvdgvevhnaktkpreeqynstyrvvsvltvlhqdwl |
| | ngkeykckvsnkalpapiektiskakgqprepqvytlppsreemtknqvsltclvkgfypsdiavewesngqpennyktt |
| | ppvldsdgsfflyskltvdksrwqqgnvfscsvmhealhnhytqkslslspgrpqgfgpp (SEQ ID NO: 227) |
| TNT-205 | evqllesgggvvqpgrslrlscaasgftfssygmhwvrqapgkglewvavisydgsnkyyadsvkgrftisrdnskntlylq |
| with Q tag | mnslraedtavyycakdygynwnffdywgqgtlvtvsSastkgpsvfplapsskstsggtaalgclvkdyfpepvtvswn |
| | sgaltsgvhtfpavlqssglyslssvvtvpssslgtqtyicnvnhkpsntkvdkkvepkscdkthtcppcpapellggpsvflf |
| | ppkpkdtlmisrtpevtcvvvdvshedpevkfnwyvdgvevhnaktkpreeqynstyrvvsvltvlhqdwlngkeykc |
| | kvsnkalpapiektiskakgqprepqvytlppsreemtknqvsltclvkgfypsdiavewesngqpennykttppvldsdg |
| | sfflyskltvdksrwqqgnvfscsvmhealhnhytqkslslspgrpqgfgpp (SEQ ID NO: 228) |
| TNT-206 | qvqlqesgpglvkpsgtlsltctvsggsissggyywawirqppgkglewigsiyytgstyynpslksrvtiyvdtsknqfslkl |
| with Q tag | ssvtaadtavyycardpfpeppdafdiwgqgtmvtvsSastkgpsvfplapsskstsggtaalgclvkdyfpepvtvswns |
| | galtsgvhtfpavlqssglyslssvvtvpssslgtqtyicnvnhkpsntkvdkkvepkscdkthtcppcpapellggpsvflfp |
| | pkpkdtlmisrtpevtcvvvdvshedpevkfnwyvdgvevhnaktkpreeqynstyrvvsvltvlhqdwlngkeykck |
| | vsnkalpapiektiskakgqprepqvytlppsreemtknqvsltclvkgfypsdiavewesngqpennykttppvldsdgs |
| | fflyskltvdksrwqqgnvfscsvmhealhnhytqkslslspgrpqgfgpp (SEQ ID NO: 229) |
| TNT-207 | evqlvqsgaevkkpgasvkvsckasgytftsydinwvrqapgqglewmggiipifgtanyaqkfqgrvtitadeststaym |
| with Q tag | elrslrsedtavyycastavagfgidywgqgtlvtvsSastkgpsvfplapsskstsggtaalgclvkdyfpepvtvswnsgal |
| | tsgvhtfpavlqssglyslssvvtvpssslgtqtyicnvnhkpsntkvdkkvepkscdkthtcppcpapellggpsvflfppk |
| | pkdtlmisrtpevtcvvvdvshedpevkfnwyvdgvevhnaktkpreeqynstyrvvsvltvlhqdwlngkeykckvsn |
| | kalpapiektiskakgqprepqvytlppsreemtknqvsltclvkgfypsdiavewesngqpennykttppvldsdgsffly |
| | skltvdksrwqqgnvfscsvmhealhnhytqkslslspgrpqgfgpp (SEQ ID NO: 230) |
| TNT-208 | evqlvqsgaevkkpgssvkvsckasggtfssyaiswvrqapgqglewmggiipifgtanyaqkfqgrvtitadkststaym |
| with Q tag | elsslrsedtavyycaagyyydssgyyhqpyyfdywgqgtlvtvsSastkgpsvfplapsskstsggtaalgclvkdyfpep |
| | vtvswnsgaltsgvhtfpavlqssglyslssvvtvpssslgtqtyicnvnhkpsntkvdkkvepkscdkthtcppcpapellg |
| | gpsvflfppkpkdtlmisrtpevtcvvvdvshedpevkfnwyvdgvevhnaktkpreeqynstyrvvsvltvlhqdwlng |
| | keykckvsnkalpapiektiskakgqprepqvytlppsreemtknqvsltclvkgfypsdiavewesngqpennykttpp |
| | vldsdgsfflyskltvdksrwqqgnvfscsvmhealhnhytqkslslspgrpqgfgpp (SEQ ID NO: 231) |
| TNT-209 | evqlvesggglvqpggslrlscaasgftfsdyymswirqapgkglewvsgiswnsgsigyadsvkgrftisrdnskntlylq |
| with Q tag | mnslraedtavyycassgswylpldywgqgtlvtvsSastkgpsvfplapsskstsggtaalgclvkdyfpepvtvswnsg |
| | altsgvhtfpavlqssglyslssvvtvpssslgtqtyicnvnhkpsntkvdkkvepkscdkthtcppcpapellggpsvflfpp |
| | kpkdtlmisrtpevtcvvvdvshedpevkfnwyvdgvevhnaktkpreeqynstyrvvsvltvlhqdwlngkeykckvs |
| | nkalpapiektiskakgqprepqvytlppsreemtknqvsltclvkgfypsdiavewesngqpennykttppvldsdgsffl |
| | yskltvdksrwqqgnvfscsvmhealhnhytqkslslspgrpqgfgpp (SEQ ID NO: 232) |
| TNT-210 | evqlvqsgggvvqpgrslrlscaasgftfssyamhwvrqapgkglewvavisydgsnkyyadsvkgrftisrdnskntlyl |
| with Q tag | qmnslraedtavyycaratlqlwlplfdywgqgtlvtvsSastkgpsvfplapsskstsggtaalgclvkdyfpepvtvswn |
| | sgaltsgvhtfpavlqssglyslssvvtvpssslgtqtyicnvnhkpsntkvdkkvepkscdkthtcppcpapellggpsvflf |
| | ppkpkdtlmisrtpevtcvvvdvshedpevkfnwyvdgvevhnaktkpreeqynstyrvvsvltvlhqdwlngkeykc |
| | kvsnkalpapiektiskakgqprepqvytlppsreemtknqvsltclvkgfypsdiavewesngqpennykttppvldsdg |
| | sfflyskltvdksrwqqgnvfscsvmhealhnhytqkslslspgrpqgfgpp (SEQ ID NO: 233) |
| TNT-211 | evqlvqsgaevkkpgssvkvsckasggtfssyaiswvrqapgqglewmgwinpnsggtnyaqkfqgrvtmtrdtststa |
| with Q tag | ymelsslrsedtavyycatgyygdlyyfdywgqgtlvtvsSastkgpsvfplapsskstsggtaalgclvkdyfpepvtvsw |
| | nsgaltsgvhtfpavlqssglyslssvvtvpssslgtqtyicnvnhkpsntkvdkkvepkscdkthtcppcpapellggpsvfl |
| | fppkpkdtlmisrtpevtcvvvdvshedpevkfnwyvdgvevhnaktkpreeqynstyrvvsvltvlhqdwlngkeykc |
| | kvsnkalpapiektiskakgqprepqvytlppsreemtknqvsltclvkgfypsdiavewesngqpennykttppvldsdg |
| | sfflyskltvdksrwqqgnvfscsvmhealhnhytqkslslspgrpqgfgpp (SEQ ID NO: 234) |
| TNT-154 | evqlvesggglvqpggslrlscaasgftfssynmnwvrqapgkglewvsyissssstiyyadsvkgrftisrdnaknslslq |
| with Q tag; | mnslrdedtavyycarayyygmdvwgqgttvtvsSastkgpsvfplapsskstsggtaalgclvkdyfpepvtvswnsga |
| TNT-212 | ltsgvhtfpavlqssglyslssvvtvpssslgtqtyicnvnhkpsntkvdkkvepkscdkthtcppcpapellggpsvflfppk |
| with Q tag | pkdtlmisrtpevtcvvvdvshedpevkfnwyvdgvevhnaktkpreeqynstyrvvsvltvlhqdwlngkeykckvsn |
| | kalpapiektiskakgqprepqvytlppsreemtknqvsltclvkgfypsdiavewesngqpennykttppvldsdgsffly |
| | skltvdksrwqqgnvfscsvmhealhnhytqkslslspgrpqgfgpp (SEQ ID NO: 235) |
| TNT-317 | QVQLQESGPGLVKPSETLSLTCTVSGGSISSYFWSWIRQPPGKGLEWIGYIYYSGN |
| with Q tag | TNYNPSLKSRVTISVDMSKNQFSLRLSSVTAADTAVYYCARDFHHYDGSAYRW |
| | YFDLWGRGTLVTVSSastkgpsvfplapsskstsggtaalgclvkdyfpepvtvswnsgaltsgvhtfpavlqss |
| | glyslssvvtvpssslgtqtyicnvnhkpsntkvdkkvepkscdkthtcppcpapellggpsvflfppkpkdtlmisrtpevt |
| | cvvvdvshedpevkfnwyvdgvevhnaktkpreeqynstyrvvsvltvlhqdwlngkeykckvsnkalpapiektiska |
| | kgqprepqvytlppsreemtknqvsltclvkgfypsdiavewesngqpennykttppvldsdgsfflyskltvdksrwqqg |
| | nvfscsvmhealhnhytqkslslspgrpqgfgpp (SEQ ID NO: 1030) |
| TNT-318 | QVQLQESGPGLVKPSETLSLTCTVSGGSISSNYWTWIRQSAGKGLEWIGRIHISES |
| with Q tag | TNYNPSLKSRVTMSVDTSKNQFSLKLSSVTAADTAVYFCAREGEWKVLGAFDIW |
| | GQGTMVTVSSastkgpsvfplapsskstsggtaalgclvkdyfpepvtvswnsgaltsgvhtfpavlqssglyslssv |
| | vtvpssslgtqtyicnvnhkpsntkvdkkvepkscdkthtcppcpapellggpsvflfppkpkdtlmisrtpevtcvvvdvs |
| | hedpevkfnwyvdgvevhnaktkpreeqynstyrvvsvltvlhqdwlngkeykckvsnkalpapiektiskakgqprep |
| | qvytlppsreemtknqvsltclvkgfypsdiavewesngqpennykttppvldsdgsfflyskltvdksrwqqgnvfscsv |
| | mhealhnhytqkslslspgrpqgfgpp (SEQ ID NO: 1031) |
| TNT-320 | QVQLVQSGAEVKKPGASVKVSCKASGYTFTVYYIHWVRQAPGQGLEWMGWISP |
| with Q tag | NSGGTNYAQKFQGRVTMTRDTSISTAYMELTRLRSDDTAIFYCARQTVVTPNWG |
| | FDYWGQGTLVTVSSastkgpsvfplapsskstsggtaalgclvkdyfpepvtvswnsgaltsgvhtfpavlqssgl |
| | yslssvvtvpssslgtqtyicnvnhkpsntkvdkkvepkscdkthtcppcpapellggpsvflfppkpkdtlmisrtpevtcv |
| | vvdvshedpevkfnwyvdgvevhnaktkpreeqynstyrvvsvltvlhqdwlngkeykckvsnkalpapiektiskakg |
| | qprepqvytlppsreemtknqvsltclvkgfypsdiavewesngqpennykttppvldsdgsfflyskltvdksrwqqgnv |
| | fscsvmhealhnhytqkslslspgrpqgfgpp (SEQ ID NO: 1032) |
| TNT-321 | QVQLQESGPGLVKPSETLSLTCTVSGGSISSYYWSWIRQPPGKGLEWIGYIYYSGN |
| with Q tag | VNYNPSLKSRVTISVDASKNQFSLKLTSVTAADTAVYYCAIGGVGAQWDAFDIW |
| | GQGTMVTVSSastkgpsvfplapsskstsggtaalgclvkdyfpepvtvswnsgaltsgvhtfpavlqssglyslssv |
| | vtvpssslgtqtyicnvnhkpsntkvdkkvepkscdkthtcppcpapellggpsvflfppkpkdtlmisrtpevtcvvvdvs |
| | hedpevkfnwyvdgvevhnaktkpreeqynstyrvvsvltvlhqdwlngkeykckvsnkalpapiektiskakgqprep |
| | qvytlppsreemtknqvsltclvkgfypsdiavewesngqpennykttppvldsdgsfflyskltvdksrwqqgnvfscsv |
| | mhealhnhytqkslslspgrpqgfgpp (SEQ ID NO: 1033) |
| TNT-322 | QVQLQESGPGLVKPSATLSLTCTVSGGSISSYYWNWIRQPPGKGLEWIGYIYYSG |
| with Q tag | NTNYNPSLKSRVTISVDMSKNQFSLKLNSVTAADTAVYYCARSESYGYWFFYLW |
| | GRGTLVTVSSastkgpsvfplapsskstsggtaalgclvkdyfpepvtvswnsgaltsgvhtfpavlqssglyslssvv |
| | tvpssslgtqtyicnvnhkpsntkvdkkvepkscdkthtcppcpapellggpsvflfppkpkdtlmisrtpevtcvvvdvsh |
| | edpevkfnwyvdgvevhnaktkpreeqynstyrvvsvltvlhqdwlngkeykckvsnkalpapiektiskakgqprepq |
| | vytlppsreemtknqvsltclvkgfypsdiavewesngqpennykttppvldsdgsfflyskltvdksrwqqgnvfscsvm |
| | healhnhytqkslslspgrpqgfgpp (SEQ ID NO: 1034) |
| TNT-323 | QVQLQESGPGLVKPSETLSLTCTVSGGSISSYFWNWIRQPPGKGLEWIGYLYYIGT |
| with Q tag | TNYNPSLKSRVTISVDTSKNQFSLKLNSVTAADTAVYYCARSGNYYNDAFDIWG |
| | QGTMVTVSSastkgpsvfplapsskstsggtaalgclvkdyfpepvtvswnsgaltsgvhtfpavlqssglyslssvvt |
| | vpssslgtqtyicnvnhkpsntkvdkkvepkscdkthtcppcpapellggpsvflfppkpkdtlmisrtpevtcvvvdvshe |
| | dpevkfnwyvdgvevhnaktkpreeqynstyrvvsvltvlhqdwlngkeykckvsnkalpapiektiskakgqprepqv |
| | ytlppsreemtknqvsltclvkgfypsdiavewesngqpennykttppvldsdgsfflyskltvdksrwqqgnvfscsvmh |
| | ealhnhytqkslslspgrpqgfgpp (SEQ ID NO: 1035) |
| TNT-324 | QVQLVQSGAEVKKPGASVKVSCKASGYTFTGYFIHWVRQAPGQGLEWMGWINP |
| with Q tag | NSGGTNYAQKFQGRVTMTRDTSISTAYMELSRLRSDDTAVYYCARDTFYYYYG |
| | MDVWGQGTTVTVSSastkgpsvfplapsskstsggtaalgclvkdyfpepvtvswnsgaltsgvhtfpavlqssg |
| | lyslssvvtvpssslgtqtyicnvnhkpsntkvdkkvepkscdkthtcppcpapellggpsvflfppkpkdtlmisrtpevtc |
| | vvvdvshedpevkfnwyvdgvevhnaktkpreeqynstyrvvsvltvlhqdwlngkeykckvsnkalpapiektiskak |
| | gqprepqvytlppsreemtknqvsltclvkgfypsdiavewesngqpennykttppvldsdgsfflyskltvdksrwqqgn |
| | vfscsvmhealhnhytqkslslspgrpqgfgpp (SEQ ID NO: 1036) |
| TNT-327 | QVQLQESGPGLVKPSETLSLTCTVSGGSISDYYWIWIRQPAGKGLEWIGRIYTTGG |
| with Q tag | AKYNPSLKSRVTMSIDTSKNNFSLKLSSVTAADTAVYFCARDRNWEWNFDLWG |
| | RGTLVTVSSastkgpsvfplapsskstsggtaalgclvkdyfpepvtvswnsgaltsgvhtfpavlqssglyslssvvtv |
| | pssslgtqtyicnvnhkpsntkvdkkvepkscdkthtcppcpapellggpsvflfppkpkdtlmisrtpevtcvvvdvshed |
| | pevkfnwyvdgvevhnaktkpreeqynstyrvvsvltvlhqdwlngkeykckvsnkalpapiektiskakgqprepqvy |
| | tlppsreemtknqvsltclvkgfypsdiavewesngqpennykttppvldsdgsfflyskltvdksrwqqgnvfscsvmhe |
| | alhnhytqkslslspgrpqgfgpp (SEQ ID NO: 1037) |
| TNT-328 | QVQLQESGPGLVKPSETLSLTCTVSGGSMSSYFWSWIRQPPERGLEWVGYMYYS |
| with Q tag | GNTNYNPSLKSRVTTSIDTSKNQFSLKLTSVTAADTAVYYCARSGNFYWNFDLW |
| | GRGTLVTVSSastkgpsvfplapsskstsggtaalgclvkdyfpepvtvswnsgaltsgvhtfpavlqssglyslssvv |
| | tvpssslgtqtyicnvnhkpsntkvdkkvepkscdkthtcppcpapellggpsvflfppkpkdtlmisrtpevtcvvvdvsh |
| | edpevkfnwyvdgvevhnaktkpreeqynstyrvvsvltvlhqdwlngkeykckvsnkalpapiektiskakgqprepq |
| | vytlppsreemtknqvsltclvkgfypsdiavewesngqpennykttppvldsdgsfflyskltvdksrwqqgnvfscsvm |
| | healhnhytqkslslspgrpqgfgpp (SEQ ID NO: 1038) |
| TNT-330 | EVQLVESGGGLVKPGGSLRLSCAVSGFTFSSYNMNWVRQAPGKGLEWVSSISISS |
| with Q tag | SYIYYADSVKGRFTISRDNAKNSLYLQMNSLRAEDTAVYYCARDRGLDDAFDIW |
| | GQGTVVTVSSastkgpsvfplapsskstsggtaalgclvkdyfpepvtvswnsgaltsgvhtfpavlqssglyslssvv |
| | tvpssslgtqtyicnvnhkpsntkvdkkvepkscdkthtcppcpapellggpsvflfppkpkdtlmisrtpevtcvvvdvsh |
| | edpevkfnwyvdgvevhnaktkpreeqynstyrvvsvltvlhqdwlngkeykckvsnkalpapiektiskakgqprepq |
| | vytlppsreemtknqvsltclvkgfypsdiavewesngqpennykttppvldsdgsfflyskltvdksrwqqgnvfscsvm |
| | healhnhytqkslslspgrpqgfgpp (SEQ ID NO: 1039) |
| TNT-331 | QVHLQESGPGLVKPSETLSLTCTVSGGSIRSYYWSWIRQPAGKGLEWIGRIYTSGS |
| with Q tag | TNYNPSLKSRVTMSVDTSKNRLSLKLSSVTAADTAVYYCAKENWGPSFDYWGQ |
| | GTLVTVSSastkgpsvfplapsskstsggtaalgclvkdyfpepvtvswnsgaltsgvhtfpavlqssglyslssvvtvp |
| | ssslgtqtyicnvnhkpsntkvdkkvepkscdkthtcppcpapellggpsvflfppkpkdtlmisrtpevtcvvvdvshedp |
| | evkfnwyvdgvevhnaktkpreeqynstyrvvsvltvlhqdwlngkeykckvsnkalpapiektiskakgqprepqvytl |
| | ppsreemtknqvsltclvkgfypsdiavewesngqpennykttppvldsdgsfflyskltvdksrwqqgnvfscsvmhea |
| | lhnhytqkslslspgrpqgfgpp (SEQ ID NO: 1040) |
| TNT-332 | QVQLVQSGAEAKKPGASVRVSCKTSGYTFTNYYIHWVRQAPGQGLEWMGIINPT |
| with Q tag | LDSTSYAQKFQDRVTMTRDTSTNTDYMDLSSLRFEDTAVYYCARSAGFDAFDIW |
| | GQGTMVTVSSastkgpsvfplapsskstsggtaalgclvkdyfpepvtvswnsgaltsgvhtfpavlqssglyslssv |
| | vtvpssslgtqtyicnvnhkpsntkvdkkvepkscdkthtcppcpapellggpsvflfppkpkdtlmisrtpevtcvvvdvs |
| | hedpevkfnwyvdgvevhnaktkpreeqynstyrvvsvltvlhqdwlngkeykckvsnkalpapiektiskakgqprep |
| | qvytlppsreemtknqvsltclvkgfypsdiavewesngqpennykttppvldsdgsfflyskltvdksrwqqgnvfscsv |
| | mhealhnhytqkslslspgrpqgfgpp |
| | (SEQ ID NO: 1041) |
| TNT-333 | QVQLQESGPGLVKPSETLSLTCTISGGSISSYYWSWIRQPPGKGLEWIGYIFYIGNT |
| with Q tag | NYYPSLKSRVTISVDASKNQFSLKLSSVTAADTAVYYCARSYPYWYFDLWGRGT |
| | LVTVSSastkgpsvfplapsskstsggtaalgclvkdyfpepvtvswnsgaltsgvhtfpavlqssglyslssvvtvpsssl |
| | gtqtyicnvnhkpsntkvdkkvepkscdkthtcppcpapellggpsvflfppkpkdtlmisrtpevtcvvvdvshedpevk |
| | fnwyvdgvevhnaktkpreeqynstyrvvsvltvlhqdwlngkeykckvsnkalpapiektiskakgqprepqvytlpps |
| | reemtknqvsltclvkgfypsdiavewesngqpennykttppvldsdgsfflyskltvdksrwqqgnvfscsvmhealhn |
| | hytqkslslspgrpqgfgpp (SEQ ID NO: 1042) |
| TNT-334 | EVQLVESGGGLVQPGGSLRLSCAASGFTFSRYWMHWVRQAPGKGLVWVSRINS |
| with Q tag | DGSSSNYADSVKGRFTFSRDNAKNTLYLQMNSLRAEDTAVYFCVRDNWYFDY |
| | WGQGTLVTVSSastkgpsvfplapsskstsggtaalgclvkdyfpepvtvswnsgaltsgvhtfpavlqssglyslss |
| | vvtvpssslgtqtyicnvnhkpsntkvdkkvepkscdkthtcppcpapellggpsvflfppkpkdtlmisrtpevtcvvvdv |
| | shedpevkfnwyvdgvevhnaktkpreeqynstyrvvsvltvlhqdwlngkeykckvsnkalpapiektiskakgqpre |
| | pqvytlppsreemtknqvsltclvkgfypsdiavewesngqpennykttppvldsdgsfflyskltvdksrwqqgnvfscs |
| | vmhealhnhytqkslslspgrpqgfgpp |
| | (SEQ ID NO: 1043) |
| TNT-335 | QVQLQESGPGLVKPSETLSLTCTVSGGSISSYYWSWIRQPPGKGLEWIGYIHYSGI |
| with Q tag | SNYNPSLKSRVAISLDTSNNHFSLKLTSLTAADTAVYFCARGSYWFDPWGQGTL |
| | VTVSSastkgpsvfplapsskstsggtaalgclvkdyfpepvtvswnsgaltsgvhtfpavlqssglyslssvvtvpssslgt |
| | qtyicnvnhkpsntkvdkkvepkscdkthtcppcpapellggpsvflfppkpkdtlmisrtpevtcvvvdvshedpevkfn |
| | wyvdgvevhnaktkpreeqynstyrvvsvltvlhqdwlngkeykckvsnkalpapiektiskakgqprepqvytlppsre |
| | emtknqvsltclvkgfypsdiavewesngqpennykttppvldsdgsfflyskltvdksrwqqgnvfscsvmhealhnhy |
| | tqkslslspgrpqgfgpp (SEQ ID NO: 1044) |
| TNT-336 | KVQLVESGGGLVQPGGSLRLSCEASGFTFSNYWMHWVRQVPGEGLVWVSRLN |
| with Q tag | NGGTSTRYADSVKSRFTISRDNAKNTLYLQMNSLRAEDTAVYYCVRDWYFDLW |
| | GRGTLVTVSSastkgpsvfplapsskstsggtaalgclvkdyfpepvtvswnsgaltsgvhtfpavlqssglyslssvv |
| | tvpssslgtqtyicnvnhkpsntkvdkkvepkscdkthtcppcpapellggpsvflfppkpkdtlmisrtpevtcvvvdvsh |
| | edpevkfnwyvdgvevhnaktkpreeqynstyrvvsvltvlhqdwlngkeykckvsnkalpapiektiskakgqprepq |
| | vytlppsreemtknqvsltclvkgfypsdiavewesngqpennykttppvldsdgsfflyskltvdksrwqqgnvfscsvm |
| | healhnhytqkslslspgrpqgfgpp (SEQ ID NO: 1045) |
| TNT-337 | EVQLVESGGGLVQPGGSLRLSCAASGFTFSNYDMSWVRQAPGKGLEWVATISSG |
| with Q tag | GSYTYYVDSVKGRFTISRDNAKNSLYLQMNSLRAEDTAVYYCARQELGSYYAM |
| | DYWGQGTTVTVSSastkgpsvfplapsskstsggtaalgclvkdyfpepvtvswnsgaltsgvhtfpavlqssgly |
| | slssvvtvpssslgtqtyicnvnhkpsntkvdkkvepkscdkthtcppcpapellggpsvflfppkpkdtlmisrtpevtcvv |
| | vdvshedpevkfnwyvdgvevhnaktkpreeqynstyrvvsvltvlhqdwlngkeykckvsnkalpapiektiskakgq |
| | prepqvytlppsreemtknqvsltclvkgfypsdiavewesngqpennykttppvldsdgsfflyskltvdksrwqqgnvf |
| | scsvmhealhnhytqkslslspgrpqgfgpp (SEQ ID NO: 1046) |
| TNT-412 | QVQLVQSGAEVKKPGASVKVSCKASGYTFTGYYMHWVRQAPGQGLEWMGWI |
| with Q tag | NPNSGGTNYAQKFQGRVTMTRDTSISTAYMELSRLRSDDTAVYYCARGSALHFF |
| | GVFDYWGQGTLVTVSSastkgpsvfplapsskstsggtaalgclvkdyfpepvtvswnsgaltsgvhtfpavlq |
| | ssglyslssvvtvpssslgtqtyicnvnhkpsntkvdkkvepkscdkthtcppcpapellggpsvflfppkpkdtlmisrtpe |
| | vtcvvvdvshedpevkfnwyvdgvevhnaktkpreeqynstyrvvsvltvlhqdwlngkeykckvsnkalpapiektis |
| | kakgqprepqvytlppsreemtknqvsltclvkgfypsdiavewesngqpennykttppvldsdgsfflyskltvdksrwq |
| | qgnvfscsvmhealhnhytqkslslspgrpqgfgpp (SEQ ID NO: 1047) |
| TNT-417 | QVQLVQSGAEVKKPGSSVKVSCKASGGTFSSYAISWVRQAPGQGLEWMGGIIPIF |
| with Q tag | GTANYAQKFQGRVTITADKSTSTAYMELSSLRSEDTAVYYCAAGYYYDSSGYY |
| | HQPYYFDYWGQGTLVTVSSastkgpsvfplapsskstsggtaalgclvkdyfpepvtvswnsgaltsgvhtfp |
| | avlqssglyslssvvtvpssslgtqtyicnvnhkpsntkvdkkvepkscdkthtcppcpapellggpsvflfppkpkdtlmis |
| | rtpevtcvvvdvshedpevkfnwyvdgvevhnaktkpreeqynstyrvvsvltvlhqdwlngkeykckvsnkalpapie |
| | ktiskakgqprepqvytlppsreemtknqvsltclvkgfypsdiavewesngqpennykttppvldsdgsfflyskltvdks |
| | rwqqgnvfscsvmhealhnhytqkslslspgrpqgfgpp (SEQ ID NO: 1048) |
| TNT-418 | QVQLQESGPGLVKPSETLSLTCTVSGGSISDYYWIWIRQPAGKGLEWIGRIYTTGG |
| with Q tag | AKYNPSLKSRVTMSVDTSKNQFSLKLSSVTAADTAVYFCARDRNWEWNFDLWG |
| | RGTLVTVSSastkgpsvfplapsskstsggtaalgclvkdyfpepvtvswnsgaltsgvhtfpavlqssglyslssvvtv |
| | pssslgtqtyicnvnhkpsntkvdkkvepkscdkthtcppcpapellggpsvflfppkpkdtlmisrtpevtcvvvdvshed |
| | pevkfnwyvdgvevhnaktkpreeqynstyrvvsvltvlhqdwlngkeykckvsnkalpapiektiskakgqprepqvy |
| | tlppsreemtknqvsltclvkgfypsdiavewesngqpennykttppvldsdgsfflyskltvdksrwqqgnvfscsvmhe |
| | alhnhytqkslslspgrpqgfgpp (SEQ ID NO: 1049) |
| TNT-419 | QVQLQESGPGLVKPSETLSLTCTVSGGSISDYYWSWIRQPAGKGLEWIGRIYTTG |
| with Q tag | GAKYNPSLKSRVTMSVDTSKNQFSLKLSSVTAADTAVYYCARDRNWEWNFDL |
| | WGRGTLVTVSSastkgpsvfplapsskstsggtaalgclvkdyfpepvtvswnsgaltsgvhtfpavlqssglyslss |
| | vvtvpssslgtqtyicnvnhkpsntkvdkkvepkscdkthtcppcpapellggpsvflfppkpkdtlmisrtpevtcvvvdv |
| | shedpevkfnwyvdgvevhnaktkpreeqynstyrvvsvltvlhqdwlngkeykckvsnkalpapiektiskakgqpre |
| | pqvytlppsreemtknqvsltclvkgfypsdiavewesngqpennykttppvldsdgsfflyskltvdksrwqqgnvfscs |
| | vmhealhnhytqkslslspgrpqgfgpp (SEQ ID NO: 1050) |
| TNT-420 | QVQLQESGPGLVKPSETLSLTCTVSGGSMSSYFWSWIRQPPGKGLEWIGYMYYS |
| with Q tag | GNTNYNPSLKSRVTISVDTSKNQFSLKLSSVTAADTAVYYCARSGNFYWNFDLW |
| | GRGTLVTVSSastkgpsvfplapsskstsggtaalgclvkdyfpepvtvswnsgaltsgvhtfpavlqssglyslssvv |
| | tvpssslgtqtyicnvnhkpsntkvdkkvepkscdkthtcppcpapellggpsvflfppkpkdtlmisrtpevtcvvvdvsh |
| | edpevkfnwyvdgvevhnaktkpreeqynstyrvvsvltvlhqdwlngkeykckvsnkalpapiektiskakgqprepq |
| | vytlppsreemtknqvsltclvkgfypsdiavewesngqpennykttppvldsdgsfflyskltvdksrwqqgnvfscsvm |
| | healhnhytqkslslspgrpqgfgpp (SEQ ID NO: 1051) |
| TNT-421 | QVQLQESGPGLVKPSETLSLTCTVSGGSISSYYWSWIRQPPGKGLEWIGYIFYIGN |
| with Q tag | TNYYPSLKSRVTISVDTSKNQFSLKLSSVTAADTAVYYCARSYPYWYFDLWGRG |
| | TLVTVSSastkgpsvfplapsskstsggtaalgclvkdyfpepvtvswnsgaltsgvhtfpavlqssglyslssvvtvpss |
| | slgtqtyicnvnhkpsntkvdkkvepkscdkthtcppcpapellggpsvflfppkpkdtlmisrtpevtcvvvdvshedpe |
| | vkfnwyvdgvevhnaktkpreeqynstyrvvsvltvlhqdwlngkeykckvsnkalpapiektiskakgqprepqvytlp |
| | psreemtknqvsltclvkgfypsdiavewesngqpennykttppvldsdgsfflyskltvdksrwqqgnvfscsvmheal |
| | hnhytqkslslspgrpqgfgpp (SEQ ID NO: 1052) |
| TNT-413 | EVQLVQSGAEVKKPGSSVKVSCKASGYTFTGYYMHWVRQAPGQGLEWMGWIN |
| with Q tag | PNSGGTNYAQKFQGRVTMTRDTSISTAYMELRSLRSDDTAVYYCARGSALHFFG |
| | VFDYWGQGTLVTVSSastkgpsvfplapsskstsggtaalgclvkdyfpepvtvswnsgaltsgvhtfpavlqss |
| | glyslssvvtvpssslgtqtyicnvnhkpsntkvdkkvepkscdkthtcppcpapellAgpsvflfppkpkdtlmisrtpevt |
| | cvvvdvshedpevkfnwyvdgvevhnaktkpreeqynstyrvvsvltvlhqdwlngkeykckvsnkalpapiektiska |
| | kgqprepqvytlppsreemtknqvsltclvkgfypsdiavewesngqpennykttppvldsdgsfflyskltvdksrwqqg |
| | nvfscsvmhealhnhytqkslslspgrpqgfgpp (SEQ ID NO: 1053) |
| TNT-422 | EVQLVQSGAEVKKPGASVKVSCKASGYTFTSYDINWVRQAPGQGLEWMGGIIPI |
| with Q tag | FGTANYAQKFQGRVTITADESTSTAYMELRSLRSEDTAVYYCASTAVAGFGIDY |
| | WGQGTLVTVSSastkgpsvfplapsskstsggtaalgclvkdyfpepvtvswnsgaltsgvhtfpavlqssglyslss |
| | vvtvpssslgtqtyicnvnhkpsntkvdkkvepkscdkthtcppcpapellAgpsvflfppkpkdtlmisrtpevtcvvvd |
| | vshedpevkfnwyvdgvevhnaktkpreeqynstyrvvsvltvlhqdwlngkeykckvsnkalpapiektiskakgqpr |
| | epqvytlppsreemtknqvsltclvkgfypsdiavewesngqpennykttppvldsdgsfflyskltvdksrwqqgnvfsc |
| | svmhealhnhytqkslslspgrpqgfgpp (SEQ ID NO: 1054) |
| TNT-423 | EVQLVQSGAEVKKPGSSVKVSCKASGGTFSSYAISWVRQAPGQGLEWMGGIIPIF |
| with Q tag | GTANYAQKFQGRVTITADKSTSTAYMELSSLRSEDTAVYYCAAGYYYDSSGYY |
| | HQPYYFDYWGQGTLVTVSSastkgpsvfplapsskstsggtaalgclvkdyfpepvtvswnsgaltsgvhtfp |
| | avlqssglyslssvvtvpssslgtqtyicnvnhkpsntkvdkkvepkscdkthtcppcpapellAgpsvflfppkpkdtlmi |
| | srtpevtcvvvdvshedpevkfnwyvdgvevhnaktkpreeqynstyrvvsvltvlhqdwlngkeykckvsnkalpapi |
| | ektiskakgqprepqvytlppsreemtknqvsltclvkgfypsdiavewesngqpennykttppvldsdgsfflyskltvdk |
| | srwqqgnvfscsvmhealhnhytqkslslspgrpqgfgpp (SEQ ID NO: 1055) |
| TNT-424 | EVQLVQSGGGVVQPGRSLRLSCAASGFTFSSYAMHWVRQAPGKGLEWVAVISY |
| with Q tag | DGSNKYYADSVKGRFTISRDNSKNTLYLQMNSLRAEDTAVYYCARATLQLWLP |
| | LFDYWGQGTLVTVSSastkgpsvfplapsskstsggtaalgclvkdyfpepvtvswnsgaltsgvhtfpavlqss |
| | glyslssvvtvpssslgtqtyicnvnhkpsntkvdkkvepkscdkthtcppcpapellAgpsvflfppkpkdtlmisrtpevt |
| | cvvvdvshedpevkfnwyvdgvevhnaktkpreeqynstyrvvsvltvlhqdwlngkeykckvsnkalpapiektiska |
| | kgqprepqvytlppsreemtknqvsltclvkgfypsdiavewesngqpennykttppvldsdgsfflyskltvdksrwqqg |
| | nvfscsvmhealhnhytqkslslspgrpqgfgpp (SEQ ID NO: 1056) |
| TNT-425 | QVQLQESGPGLVKPSETLSLTCTVSGGSISDYYWIWIRQPAGKGLEWIGRIYTTGG |
| with Q tag | AKYNPSLKSRVTMSIDTSKNNFSLKLSSVTAADTAVYFCARDRNWEWNFDLWG |
| | RGTLVTVSSastkgpsvfplapsskstsggtaalgclvkdyfpepvtvswnsgaltsgvhtfpavlqssglyslssvvtv |
| | pssslgtqtyicnvnhkpsntkvdkkvepkscdkthtcppcpapellAgpsvflfppkpkdtlmisrtpevtcvvvdvshe |
| | dpevkfnwyvdgvevhnaktkpreeqynstyrvvsvltvlhqdwlngkeykckvsnkalpapiektiskakgqprepqv |
| | ytlppsreemtknqvsltclvkgfypsdiavewesngqpennykttppvldsdgsfflyskltvdksrwqqgnvfscsvmh |
| | ealhnhytqkslslspgrpqgfgpp (SEQ ID NO: 1057) |
| TNT-426 | QVQLQESGPGLVKPSETLSLTCTVSGGSMSSYFWSWIRQPPERGLEWVGYMYYS |
| with Q tag | GNTNYNPSLKSRVTTSIDTSKNQFSLKLTSVTAADTAVYYCARSGNFYWNFDLW |
| | GRGTLVTVSSastkgpsvfplapsskstsggtaalgclvkdyfpepvtvswnsgaltsgvhtfpavlqssglyslssvv |
| | tvpssslgtqtyicnvnhkpsntkvdkkvepkscdkthtcppcpapellAgpsvflfppkpkdtlmisrtpevtcvvvdvsh |
| | edpevkfnwyvdgvevhnaktkpreeqynstyrvvsvltvlhqdwlngkeykckvsnkalpapiektiskakgqprepq |
| | vytlppsreemtknqvsltclvkgfypsdiavewesngqpennykttppvldsdgsfflyskltvdksrwqqgnvfscsvm |
| | healhnhytqkslslspgrpqgfgpp (SEQ ID NO: 1058) |
| TNT-427 | QVQLQESGPGLVKPSETLSLTCTISGGSISSYYWSWIRQPPGKGLEWIGYIFYIGNT |
| with Q tag | NYYPSLKSRVTISVDASKNQFSLKLSSVTAADTAVYYCARSYPYWYFDLWGRGT |
| | LVTVSSastkgpsvfplapsskstsggtaalgclvkdyfpepvtvswnsgaltsgvhtfpavlqssglyslssvvtvpsssl |
| | gtqtyicnvnhkpsntkvdkkvepkscdkthtcppcpapellAgpsvflfppkpkdtlmisrtpevtcvvvdvshedpev |
| | kfnwyvdgvevhnaktkpreeqynstyrvvsvltvlhqdwlngkeykckvsnkalpapiektiskakgqprepqvytlpp |
| | sreemtknqvsltclvkgfypsdiavewesngqpennykttppvldsdgsfflyskltvdksrwqqgnvfscsvmhealh |
| | nhytqkslslspgrpqgfgpp (SEQ ID NO: 1059) |
| TNT-414 | QVQLVQSGAEVKKPGASVKVSCKASGYTFTGYYMHWVRQAPGQGLEWMGWI |
| with Q tag | NPNSGGTNYAQKFQGRVTMTRDTSISTAYMELSRLRSDDTAVYYCARGSALHFF |
| | GVFDYWGQGTLVTVSSastkgpsvfplapsskstsggtaalgclvkdyfpepvtvswnsgaltsgvhtfpavlq |
| | ssglyslssvvtvpssslgtqtyicnvnhkpsntkvdkkvepkscdkthtcppcpapellAgpsvflfppkpkdtlmisrtpe |
| | vtcvvvdvshedpevkfnwyvdgvevhnaktkpreeqynstyrvvsvltvlhqdwlngkeykckvsnkalpapiektis |
| | kakgqprepqvytlppsreemtknqvsltclvkgfypsdiavewesngqpennykttppvldsdgsfflyskltvdksrwq |
| | qgnvfscsvmhealhnhytqkslslspgrpqgfgpp (SEQ ID NO: 1060) |
| TNT-428 | QVQLVQSGAEVKKPGSSVKVSCKASGGTFSSYAISWVRQAPGQGLEWMGGIIPIF |
| with Q tag | GTANYAQKFQGRVTITADKSTSTAYMELSSLRSEDTAVYYCAAGYYYDSSGYY |
| | HQPYYFDYWGQGTLVTVSSastkgpsvfplapsskstsggtaalgclvkdyfpepvtvswnsgaltsgvhtfp |
| | avlqssglyslssvvtvpssslgtqtyicnvnhkpsntkvdkkvepkscdkthtcppcpapellAgpsvflfppkpkdtlmi |
| | srtpevtcvvvdvshedpevkfnwyvdgvevhnaktkpreeqynstyrvvsvltvlhqdwlngkeykckvsnkalpapi |
| | ektiskakgqprepqvytlppsreemtknqvsltclvkgfypsdiavewesngqpennykttppvldsdgsfflyskltvdk |
| | srwqqgnvfscsvmhealhnhytqkslslspgrpqgfgpp (SEQ ID NO: 1061) |
| TNT-429 | QVQLQESGPGLVKPSETLSLTCTVSGGSISDYYWIWIRQPAGKGLEWIGRIYTTGG |
| with Q tag | AKYNPSLKSRVTMSVDTSKNQFSLKLSSVTAADTAVYFCARDRNWEWNFDLWG |
| | RGTLVTVSSastkgpsvfplapsskstsggtaalgclvkdyfpepvtvswnsgaltsgvhtfpavlqssglyslssvvtv |
| | pssslgtqtyicnvnhkpsntkvdkkvepkscdkthtcppcpapellAgpsvflfppkpkdtlmisrtpevtcvvvdvshe |
| | dpevkfnwyvdgvevhnaktkpreeqynstyrvvsvltvlhqdwlngkeykckvsnkalpapiektiskakgqprepqv |
| | ytlppsreemtknqvsltclvkgfypsdiavewesngqpennykttppvldsdgsfflyskltvdksrwqqgnvfscsvmh |
| | ealhnhytqkslslspgrpqgfgpp (SEQ ID NO: 1062) |
| TNT-430 | QVQLQESGPGLVKPSETLSLTCTVSGGSISDYYWSWIRQPAGKGLEWIGRIYTTG |
| with Q tag | GAKYNPSLKSRVTMSVDTSKNQFSLKLSSVTAADTAVYYCARDRNWEWNFDL |
| | WGRGTLVTVSSastkgpsvfplapsskstsggtaalgclvkdyfpepvtvswnsgaltsgvhtfpavlqssglyslss |
| | vvtvpssslgtqtyicnvnhkpsntkvdkkvepkscdkthtcppcpapellAgpsvflfppkpkdtlmisrtpevtcvvvd |
| | vshedpevkfnwyvdgvevhnaktkpreeqynstyrvvsvltvlhqdwlngkeykckvsnkalpapiektiskakgqpr |
| | epqvytlppsreemtknqvsltclvkgfypsdiavewesngqpennykttppvldsdgsfflyskltvdksrwqqgnvfsc |
| | svmhealhnhytqkslslspgrpqgfgpp (SEQ ID NO: 1063) |
| TNT-431 | QVQLQESGPGLVKPSETLSLTCTVSGGSMSSYFWSWIRQPPGKGLEWIGYMYYS |
| with Q tag | GNTNYNPSLKSRVTISVDTSKNQFSLKLSSVTAADTAVYYCARSGNFYWNFDLW |
| | GRGTLVTVSSastkgpsvfplapsskstsggtaalgclvkdyfpepvtvswnsgaltsgvhtfpavlqssglyslssvv |
| | tvpssslgtqtyicnvnhkpsntkvdkkvepkscdkthtcppcpapellAgpsvflfppkpkdtlmisrtpevtcvvvdvsh |
| | edpevkfnwyvdgvevhnaktkpreeqynstyrvvsvltvlhqdwlngkeykckvsnkalpapiektiskakgqprepq |
| | vytlppsreemtknqvsltclvkgfypsdiavewesngqpennykttppvldsdgsfflyskltvdksrwqqgnvfscsvm |
| | healhnhytqkslslspgrpqgfgpp (SEQ ID NO: 1064) |
| TNT-432 | QVQLQESGPGLVKPSETLSLTCTVSGGSISSYYWSWIRQPPGKGLEWIGYIFYIGN |
| with Q tag | TNYYPSLKSRVTISVDTSKNQFSLKLSSVTAADTAVYYCARSYPYWYFDLWGRG |
| | TLVTVSSastkgpsvfplapsskstsggtaalgclvkdyfpepvtvswnsgaltsgvhtfpavlqssglyslssvvtvpss |
| | slgtqtyicnvnhkpsntkvdkkvepkscdkthtcppcpapellAgpsvflfppkpkdtlmisrtpevtcvvvdvshedpe |
| | vkfnwyvdgvevhnaktkpreegynstyrvvsvltvlhqdwlngkeykckvsnkalpapiektiskakgqprepqvytlp |
| | psreemtknqvsltclvkgfypsdiavewesngqpennykttppvldsdgsfflyskltvdksrwqqgnvfscsvmheal |
| | hnhytqkslslspgrpqgfgpp (SEQ ID NO: 1065) |

TABLE 15A
Nectin-4 antibody human IgG1 heavy chain

| Antibody | Sequence |
|---|---|
| TNT-201 | evqlvqsgaevkkpgssvkvsckasgytftgyymhwvrqapgqglewmgwinpnsggtnyaqkfqgrvtmtrdtsist |
| | aymelrslrsddtavyycargsalhffgvfdywgqgtlvtvsSastkgpsvfplapsskstsggtaalgclvkdyfpepvtvs |
| | wnsgaltsgvhtfpavlqssglyslssvvtvpssslgtqtyicnvnhkpsntkvdkkvepkscdkthtcppcpapellggpsv |
| | flfppkpkdtlmisrtpevtcvvvdvshedpevkfnwyvdgvevhnaktkpreeqynstyrvvsvltvlhqdwlngkey |
| | kckvsnkalpapiektiskakgqprepqvytlppsreemtknqvsltclvkgfypsdiavewesngqpennykttppvlds |
| | dgsfflyskltvdksrwqqgnvfscsvmhealhnhytqkslslspg (SEQ ID NO: 1070) |
| TNT-202 | evqlvqsgaevkkpgssvkvsckasggtfssyaiswvrqapgqglewmggiipifgtanyaqkfqgrvtitadeststaym |
| | elsslrsedtavyycargyssswyepyyfdywgqgtlvtvsSastkgpsvfplapsskstsggtaalgclvkdyfpepvtvs |
| | wnsgaltsgvhtfpavlqssglyslssvvtvpssslgtqtyicnvnhkpsntkvdkkvepkscdkthtcppcpapellggpsv |
| | flfppkpkdtlmisrtpevtcvvvdvshedpevkfnwyvdgvevhnaktkpreeqynstyrvvsvltvlhqdwlngkey |
| | kckvsnkalpapiektiskakgqprepqvytlppsreemtknqvsltclvkgfypsdiavewesngqpennykttppvlds |
| | dgsfflyskltvdksrwqqgnvfscsvmhealhnhytqkslslspg (SEQ ID NO: 1071) |
| TNT-203 | evqlvqsggglvkpggslrlscaasgftfsdyymswvrqapgkglewvsaisgsggstyyadsvkgrftisrdnskntlylq |
| | mnslraedtavyycardegyyymdvwgkgttvtvsSastkgpsvfplapsskstsggtaalgclvkdyfpepvtvswnsg |
| | altsgvhtfpavlqssglyslssvvtvpssslgtqtyicnvnhkpsntkvdkkvepkscdkthtcppcpapellggpsvflfpp |
| | kpkdtlmisrtpevtcvvvdvshedpevkfnwyvdgvevhnaktkpreeqynstyrvvsvltvlhqdwlngkeykckvs |
| | nkalpapiektiskakgqprepqvytlppsreemtknqvsltclvkgfypsdiavewesngqpennykttppvldsdgsffl |
| | yskltvdksrwqqgnvfscsvmhealhnhytqkslslspg (SEQ ID NO: 1072) |
| TNT-204 | evqllesgggvvqpgrslrlscaasgftfssygmhwvrqapgkglewvavisydgsnkyyadsvkgrftisrdnskntlylq |
| | mnslraedtavyycakssyydyvwgsyrytdafdiwgqgtmvtvsSastkgpsvfplapsskstsggtaalgclvkdyfp |
| | epvtvswnsgaltsgvhtfpavlqssglyslssvvtvpssslgtqtyicnvnhkpsntkvdkkvepkscdkthtcppcpapel |
| | lggpsvflfppkpkdtlmisrtpevtcvvvdvshedpevkfnwyvdgvevhnaktkpreeqynstyrvvsvltvlhqdwl |
| | ngkeykckvsnkalpapiektiskakgqprepqvytlppsreemtknqvsltclvkgfypsdiavewesngqpennyktt |
| | ppvldsdgsfflyskltvdksrwqqgnvfscsvmhealhnhytqkslslspg (SEQ ID NO: 1073) |
| TNT-205 | evqllesgggvvqpgrslrlscaasgftfssygmhwvrqapgkglewvavisydgsnkyyadsvkgrftisrdnskntlylq |
| | mnslraedtavyycakdygynwnffdywgqgtlvtvsSastkgpsvfplapsskstsggtaalgclvkdyfpepvtvswn |
| | sgaltsgvhtfpavlqssglyslssvvtvpssslgtqtyicnvnhkpsntkvdkkvepkscdkthtcppcpapellggpsvflf |
| | ppkpkdtlmisrtpevtcvvvdvshedpevkfnwyvdgvevhnaktkpreeqynstyrvvsvltvlhqdwlngkeykc |
| | kvsnkalpapiektiskakgqprepqvytlppsreemtknqvsltclvkgfypsdiavewesngqpennykttppvldsdg |
| | sfflyskltvdksrwqggnvfscsvmhealhnhytqkslslspg (SEQ ID NO: 1074) |
| TNT-206 | qvqlqesgpglvkpsgtlsltctvsggsissggyywawirqppgkglewigsiyytgstyynpslksrvtiyvdtsknqfslkl |
| | ssvtaadtavyycardpfpeppdafdiwgqgtmvtvsSastkgpsvfplapsskstsggtaalgclvkdyfpepvtvswns |
| | galtsgvhtfpavlqssglyslssvvtvpssslgtqtyicnvnhkpsntkvdkkvepkscdkthtcppcpapellggpsvflfp |
| | pkpkdtlmisrtpevtcvvvdvshedpevkfnwyvdgvevhnaktkpreeqynstyrvvsvltvlhqdwlngkeykck |
| | vsnkalpapiektiskakgqprepqvytlppsreemtknqvsltclvkgfypsdiavewesngqpennykttppvldsdgs |
| | fflyskltvdksrwqqgnvfscsvmhealhnhytkslslspg (SEQ ID NO: 1075) |
| TNT-207 | evqlvqsgaevkkpgasvkvsckasgytftsydinwvrqapgqglewmggiipifgtanyaqkfqgrvtitadeststaym |
| | elrslrsedtavyycastavagfgidywgqgtlvtvsSastkgpsvfplapsskstsggtaalgclvkdyfpepvtvswnsgal |
| | tsgvhtfpavlqssglyslssvvtvpssslgtqtyicnvnhkpsntkvdkkvepkscdkthtcppcpapellggpsvflfppk |
| | pkdtlmisrtpevtcvvvdvshedpevkfnwyvdgvevhnaktkpreeqynstyrvvsvltvlhqdwlngkeykckvsn |
| | kalpapiektiskakgqprepqvytlppsreemtknqvsltclvkgfypsdiavewesngqpennykttppvldsdgsffly |
| | skltvdksrwqqgnvfscsvmhealhnhytqkslslspg (SEQ ID NO: 1076) |
| TNT-208 | evqlvqsgaevkkpgssvkvsckasggtfssyaiswvrqapgqglewmggiipifgtanyaqkfqgrvtitadkststaym |
| | elsslrsedtavyycaagyyydssgyyhqpyyfdywgqgtlvtvsSastkgpsvfplapsskstsggtaalgclvkdyfpep |
| | vtvswnsgaltsgvhtfpavlqssglyslssvvtvpssslgtqtyicnvnhkpsntkvdkkvepkscdkthtcppcpapellg |
| | gpsvflfppkpkdtlmisrtpevtcvvvdvshedpevkfnwyvdgvevhnaktkpreeqynstyrvvsvltvlhqdwlng |
| | keykckvsnkalpapiektiskakgqprepqvytlppsreemtknqvsltclvkgfypsdiavewesngqpennykttpp |
| | vldsdgsfflyskltvdksrwqqgnvfscsvmhealhnhytqkslslspg (SEQ ID NO: 1077) |
| TNT-209 | evqlvesggglvqpggslrlscaasgftfsdyymswirqapgkglewvsgiswnsgsigyadsvkgrftisrdnskntlylq |
| | mnslraedtavyycassgswylpldywgqgtlvtvsSastkgpsvfplapsskstsggtaalgclvkdyfpepvtvswnsg |
| | altsgvhtfpavlqssglyslssvvtvpssslgtqtyicnvnhkpsntkvdkkvepkscdkthtcppcpapellggpsvflfpp |
| | kpkdtlmisrtpevtcvvvdvshedpevkfnwyvdgvevhnaktkpreeqynstyrvvsvltvlhqdwlngkeykckvs |
| | nkalpapiektiskakgqprepqvytlppsreemtknqvsltclvkgfypsdiavewesngqpennykttppvldsdgsffl |
| | yskltvdksrwqqgnvfscsvmhealhnhytqkslslspg (SEQ ID NO: 1078) |
| TNT-210 | evqlvqsgggvvqpgrslrlscaasgftfssyamhwvrqapgkglewvavisydgsnkyyadsvkgrftisrdnskntlyl |
| | qmnslraedtavyycaratlqlwlplfdywgqgtlvtvsSastkgpsvfplapsskstsggtaalgclvkdyfpepvtvswn |
| | sgaltsgvhtfpavlqssglyslssvvtvpssslgtqtyicnvnhkpsntkvdkkvepkscdkthtcppcpapellggpsvflf |
| | ppkpkdtlmisrtpevtcvvvdvshedpevkfnwyvdgvevhnaktkpreeqynstyrvvsvltvlhqdwlngkeykc |
| | kvsnkalpapiektiskakgqprepqvytlppsreemtknqvsltclvkgfypsdiavewesngqpennykttppvldsdg |
| | sfflyskltvdksrwqqgnvfscsvmhealhnhytqkslslspg (SEQ ID NO: 1079) |
| TNT-211 | evqlvqsgaevkkpgssvkvsckasggtfssyaiswvrqapgqglewmgwinpnsggtnyaqkfqgrvtmtrdtststa |
| | ymelsslrsedtavyycatgyygdlyyfdywgqgtlvtvsSastkgpsvfplapsskstsggtaalgclvkdyfpepvtvsw |
| | nsgaltsgvhtfpavlqssglyslssvvtvpssslgtqtyicnvnhkpsntkvdkkvepkscdkthtcppcpapellggpsvfl |
| | fppkpkdtlmisrtpevtcvvvdvshedpevkfnwyvdgvevhnaktkpreeqynstyrvvsvltvlhqdwlngkeykc |
| | kvsnkalpapiektiskakgqprepqvytlppsreemtknqvsltclvkgfypsdiavewesngqpennykttppvldsdg |
| | sfflyskltvdksrwqqgnvfscsvmhealhnhytqkslslspg (SEQ ID NO: 1080) |
| TNT-154; | evqlvesggglvqpggslrlscaasgftfssynmnwvrqapgkglewvsyissssstiyyadsvkgrftisrdnaknslslq |
| TNT-212 | mnslrdedtavyycarayyygmdvwgqgttvtvsSastkgpsvfplapsskstsggtaalgclvkdyfpepvtvswnsga |
| | ltsgvhtfpavlqssglyslssvvtvpssslgtqtyicnvnhkpsntkvdkkvepkscdkthtcppcpapellggpsvflfppk |
| | pkdtlmisrtpevtcvvvdvshedpevkfnwyvdgvevhnaktkpreeqynstyrvvsvltvlhqdwlngkeykckvsn |
| | kalpapiektiskakgqprepqvytlppsreemtknqvsltclvkgfypsdiavewesngqpennykttppvldsdgsffly |
| | skltvdksrwqqgnvfscsvmhealhnhytqkslslspg (SEQ ID NO: 1081) |
| TNT-317 | QVQLQESGPGLVKPSETLSLTCTVSGGSISSYFWSWIRQPPGKGLEWIGYIYYSGN |
| | TNYNPSLKSRVTISVDMSKNQFSLRLSSVTAADTAVYYCARDFHHYDGSAYRW |
| | YFDLWGRGTLVTVSSastkgpsvfplapsskstsggtaalgclvkdyfpepvtvswnsgaltsgvhtfpavlqss |
| | glyslssvvtvpssslgtqtyicnvnhkpsntkvdkkvepkscdkthtcppcpapellggpsvflfppkpkdtlmisrtpevt |
| | cvvvdvshedpevkfnwyvdgvevhnaktkpreeqynstyrvvsvltvlhqdwlngkeykckvsnkalpapiektiska |
| | kgqprepqvytlppsreemtknqvsltclvkgfypsdiavewesngqpennykttppvldsdgsfflyskltvdksrwqqg |
| | nvfscsvmhealhnhytqkslslspg (SEQ ID NO: 1082) |
| TNT-318 | QVQLQESGPGLVKPSETLSLTCTVSGGSISSNYWTWIRQSAGKGLEWIGRIHISES |
| | TNYNPSLKSRVTMSVDTSKNQFSLKLSSVTAADTAVYFCAREGEWKVLGAFDIW |
| | GQGTMVTVSSastkgpsvfplapsskstsggtaalgclvkdyfpepvtvswnsgaltsgvhtfpavlqssglyslssv |
| | vtvpssslgtqtyicnvnhkpsntkvdkkvepkscdkthtcppcpapellggpsvflfppkpkdtlmisrtpevtcvvvdvs |
| | hedpevkfnwyvdgvevhnaktkpreeqynstyrvvsvltvlhqdwlngkeykckvsnkalpapiektiskakgqprep |
| | qvytlppsreemtknqvsltclvkgfypsdiavewesngqpennykttppvldsdgsfflyskltvdksrwqqgnvfscsv |
| | mhealhnhytqkslslspg (SEQ ID NO: 1083) |
| TNT-320 | QVQLVQSGAEVKKPGASVKVSCKASGYTFTVYYIHWVRQAPGQGLEWMGWISP |
| | NSGGTNYAQKFQGRVTMTRDTSISTAYMELTRLRSDDTAIFYCARQTVVTPNWG |
| | FDYWGQGTLVTVSSastkgpsvfplapsskstsggtaalgclvkdyfpepvtvswnsgaltsgvhtfpavlqssgl |
| | yslssvvtvpssslgtqtyicnvnhkpsntkvdkkvepkscdkthtcppcpapellggpsvflfppkpkdtlmisrtpevtcv |
| | vvdvshedpevkfnwyvdgvevhnaktkpreeqynstyrvvsvltvlhqdwlngkeykckvsnkalpapiektiskakg |
| | qprepqvytlppsreemtknqvsltclvkgfypsdiavewesngqpennykttppvldsdgsfflyskltvdksrwqqgnv |
| | fscsvmhealhnhytqkslslspg (SEQ ID NO: 1084) |
| TNT-321 | QVQLQESGPGLVKPSETLSLTCTVSGGSISSYYWSWIRQPPGKGLEWIGYIYYSGN |
| | VNYNPSLKSRVTISVDASKNQFSLKLTSVTAADTAVYYCAIGGVGAQWDAFDIW |
| | GQGTMVTVSSastkgpsvfplapsskstsggtaalgclvkdyfpepvtvswnsgaltsgvhtfpavlqssglyslssv |
| | vtvpssslgtqtyicnvnhkpsntkvdkkvepkscdkthtcppcpapellggpsvflfppkpkdtlmisrtpevtcvvvdvs |
| | hedpevkfnwyvdgvevhnaktkpreeqynstyrvvsvltvlhqdwlngkeykckvsnkalpapiektiskakgqprep |
| | qvytlppsreemtknqvsltclvkgfypsdiavewesngqpennykttppvldsdgsfflyskltvdksrwqqgnvfscsv |
| | mhealhnhytqkslslspg (SEQ ID NO: 1085) |
| TNT-322 | QVQLQESGPGLVKPSATLSLTCTVSGGSISSYYWNWIRQPPGKGLEWIGYIYYSG |
| | NTNYNPSLKSRVTISVDMSKNQFSLKLNSVTAADTAVYYCARSESYGYWFFYLW |
| | GRGTLVTVSSastkgpsvfplapsskstsggtaalgclvkdyfpepvtvswnsgaltsgvhtfpavlqssglyslssvv |
| | tvpssslgtqtyicnvnhkpsntkvdkkvepkscdkthtcppcpapellggpsvflfppkpkdtlmisrtpevtcvvvdvsh |
| | edpevkfnwyvdgvevhnaktkpreeqynstyrvvsvltvlhqdwlngkeykckvsnkalpapiektiskakgqprepq |
| | vytlppsreemtknqvsltclvkgfypsdiavewesngqpennykttppvldsdgsfflyskltvdksrwqqgnvfscsvm |
| | healhnhytqkslslspg (SEQ ID NO: 1086) |
| TNT-323 | QVQLQESGPGLVKPSETLSLTCTVSGGSISSYFWNWIRQPPGKGLEWIGYLYYIGT |
| | TNYNPSLKSRVTISVDTSKNQFSLKLNSVTAADTAVYYCARSGNYYNDAFDIWG |
| | QGTMVTVSSastkgpsvfplapsskstsggtaalgclvkdyfpepvtvswnsgaltsgvhtfpavlqssglyslssvvt |
| | vpssslgtqtyicnvnhkpsntkvdkkvepkscdkthtcppcpapellggpsvflfppkpkdtlmisrtpevtcvvvdvshe |
| | dpevkfnwyvdgvevhnaktkpreeqynstyrvvsvltvlhqdwlngkeykckvsnkalpapiektiskakgqprepqv |
| | ytlppsreemtknqvsltclvkgfypsdiavewesngqpennykttppvldsdgsfflyskltvdksrwqqgnvfscsvmh |
| | ealhnhytqkslslspg (SEQ ID NO: 1087) |
| TNT-324 | QVQLVQSGAEVKKPGASVKVSCKASGYTFTGYFIHWVRQAPGQGLEWMGWINP |
| | NSGGTNYAQKFQGRVTMTRDTSISTAYMELSRLRSDDTAVYYCARDTFYYYYG |
| | MDVWGQGTTVTVSSastkgpsvfplapsskstsggtaalgclvkdyfpepvtvswnsgaltsgvhtfpavlqssg |
| | lyslssvvtvpssslgtqtyicnvnhkpsntkvdkkvepkscdkthtcppcpapellggpsvflfppkpkdtlmisrtpevtc |
| | vvvdvshedpevkfnwyvdgvevhnaktkpreeqynstyrvvsvltvlhqdwlngkeykckvsnkalpapiektiskak |
| | gqprepqvytlppsreemtknqvsltclvkgfypsdiavewesngqpennykttppvldsdgsfflyskltvdksrwqqgn |
| | vfscsvmhealhnhytqkslslspg (SEQ ID NO: 1088) |
| TNT-327 | QVQLQESGPGLVKPSETLSLTCTVSGGSISDYYWIWIRQPAGKGLEWIGRIYTTGG |
| | AKYNPSLKSRVTMSIDTSKNNFSLKLSSVTAADTAVYFCARDRNWEWNFDLWG |
| | RGTLVTVSSastkgpsvfplapsskstsggtaalgclvkdyfpepvtvswnsgaltsgvhtfpavlqssglyslssvvtv |
| | pssslgtqtyicnvnhkpsntkvdkkvepkscdkthtcppcpapellggpsvflfppkpkdtlmisrtpevtcvvvdvshed |
| | pevkfnwyvdgvevhnaktkpreeqynstyrvvsvltvlhqdwlngkeykckvsnkalpapiektiskakgqprepqvy |
| | tlppsreemtknqvsltclvkgfypsdiavewesngqpennykttppvldsdgsfflyskltvdksrwqqgnvfscsvmhe |
| | alhnhytqkslslspg (SEQ ID NO: 1089) |
| TNT-328 | QVQLQESGPGLVKPSETLSLTCTVSGGSMSSYFWSWIRQPPERGLEWVGYMYYS |
| | GNTNYNPSLKSRVTTSIDTSKNQFSLKLTSVTAADTAVYYCARSGNFYWNFDLW |
| | GRGTLVTVSSastkgpsvfplapsskstsggtaalgclvkdyfpepvtvswnsgaltsgvhtfpavlqssglyslssvv |
| | tvpssslgtqtyicnvnhkpsntkvdkkvepkscdkthtcppcpapellggpsvflfppkpkdtlmisrtpevtcvvvdvsh |
| | edpevkfnwyvdgvevhnaktkpreeqynstyrvvsvltvlhqdwlngkeykckvsnkalpapiektiskakgqprepq |
| | vytlppsreemtknqvsltclvkgfypsdiavewesngqpennykttppvldsdgsfflyskltvdksrwqqgnvfscsvm |
| | healhnhytqkslslspg (SEQ ID NO: 1090) |
| TNT-330 | EVQLVESGGGLVKPGGSLRLSCAVSGFTFSSYNMNWVRQAPGKGLEWVSSISISS |
| | SYIYYADSVKGRFTISRDNAKNSLYLQMNSLRAEDTAVYYCARDRGLDDAFDIW |
| | GQGTVVTVSSastkgpsvfplapsskstsggtaalgclvkdyfpepvtvswnsgaltsgvhtfpavlqssglyslssvv |
| | tvpssslgtqtyicnvnhkpsntkvdkkvepkscdkthtcppcpapellggpsvflfppkpkdtlmisrtpevtcvvvdvsh |
| | edpevkfnwyvdgvevhnaktkpreeqynstyrvvsvltvlhqdwlngkeykckvsnkalpapiektiskakgqprepq |
| | vytlppsreemtknqvsltclvkgfypsdiavewesngqpennykttppvldsdgsfflyskltvdksrwqqgnvfscsvm |
| | healhnhytqkslslspg (SEQ ID NO: 1091) |
| TNT-331 | QVHLQESGPGLVKPSETLSLTCTVSGGSIRSYYWSWIRQPAGKGLEWIGRIYTSGS |
| | TNYNPSLKSRVTMSVDTSKNRLSLKLSSVTAADTAVYYCAKENWGPSFDYWGQ |
| | GTLVTVSSastkgpsvfplapsskstsggtaalgclvkdyfpepvtvswnsgaltsgvhtfpavlqssglyslssvvtvp |
| | ssslgtqtyicnvnhkpsntkvdkkvepkscdkthtcppcpapellggpsvflfppkpkdtlmisrtpevtcvvvdvshedp |
| | evkfnwyvdgvevhnaktkpreeqynstyrvvsvltvlhqdwlngkeykckvsnkalpapiektiskakgqprepqvytl |
| | ppsreemtknqvsltclvkgfypsdiavewesngqpennykttppvldsdgsfflyskltvdksrwqqgnvfscsvmhea |
| | lhnhytqkslslspg (SEQ ID NO: 1092) |
| TNT-332 | QVQLVQSGAEAKKPGASVRVSCKTSGYTFTNYYIHWVRQAPGQGLEWMGIINPT |
| | LDSTSYAQKFQDRVTMTRDTSTNTDYMDLSSLRFEDTAVYYCARSAGFDAFDIW |
| | GQGTMVTVSSastkgpsvfplapsskstsggtaalgclvkdyfpepvtvswnsgaltsgvhtfpavlqssglyslssv |
| | vtvpssslgtqtyicnvnhkpsntkvdkkvepkscdkthtcppcpapellggpsvflfppkpkdtlmisrtpevtcvvvdvs |
| | hedpevkfnwyvdgvevhnaktkpreeqynstyrvvsvltvlhqdwlngkeykckvsnkalpapiektiskakgqprep |
| | qvytlppsreemtknqvsltclvkgfypsdiavewesngqpennykttppvldsdgsfflyskltvdksrwqqgnvfscsv |
| | mhealhnhytqkslslspg |
| | (SEQ ID NO: 1093) |
| TNT-333 | QVQLQESGPGLVKPSETLSLTCTISGGSISSYYWSWIRQPPGKGLEWIGYIFYIGNT |
| | NYYPSLKSRVTISVDASKNQFSLKLSSVTAADTAVYYCARSYPYWYFDLWGRGT |
| | LVTVSSastkgpsvfplapsskstsggtaalgclvkdyfpepvtvswnsgaltsgvhtfpavlqssglyslssvvtvpsssl |
| | gtqtyicnvnhkpsntkvdkkvepkscdkthtcppcpapellggpsvflfppkpkdtlmisrtpevtcvvvdvshedpevk |
| | fnwyvdgvevhnaktkpreeqynstyrvvsvltvlhqdwlngkeykckvsnkalpapiektiskakgqprepqvytlpps |
| | reemtknqvsltclvkgfypsdiavewesngqpennykttppvldsdgsfflyskltvdksrwqqgnvfscsvmhealhn |
| | hytqkslslspg (SEQ ID NO: 1094) |
| TNT-334 | EVQLVESGGGLVQPGGSLRLSCAASGFTFSRYWMHWVRQAPGKGLVWVSRINS |
| | DGSSSNYADSVKGRFTFSRDNAKNTLYLQMNSLRAEDTAVYFCVRDNWYFDY |
| | WGQGTLVTVSSastkgpsvfplapsskstsggtaalgclvkdyfpepvtvswnsgaltsgvhtfpavlqssglyslss |
| | vvtvpssslgtqtyicnvnhkpsntkvdkkvepkscdkthtcppcpapellggpsvflfppkpkdtlmisrtpevtcvvvdv |
| | shedpevkfnwyvdgvevhnaktkpreeqynstyrvvsvltvlhqdwlngkeykckvsnkalpapiektiskakgqpre |
| | pqvytlppsreemtknqvsltclvkgfypsdiavewesngqpennykttppvldsdgsfflyskltvdksrwqqgnvfscs |
| | vmhealhnhytqkslslspg |
| | (SEQ ID NO: 1095) |
| TNT-335 | QVQLQESGPGLVKPSETLSLTCTVSGGSISSYYWSWIRQPPGKGLEWIGYIHYSGI |
| | SNYNPSLKSRVAISLDTSNNHFSLKLTSLTAADTAVYFCARGSYWFDPWGQGTL |
| | VTVSSastkgpsvfplapsskstsggtaalgclvkdyfpepvtvswnsgaltsgvhtfpavlqssglyslssvvtvpssslgt |
| | qtyicnvnhkpsntkvdkkvepkscdkthtcppcpapellggpsvflfppkpkdtlmisrtpevtcvvvdvshedpevkfn |
| | wyvdgvevhnaktkpreeqynstyrvvsvltvlhqdwlngkeykckvsnkalpapiektiskakgqprepqvytlppsre |
| | emtknqvsltclvkgfypsdiavewesngqpennykttppvldsdgsfflyskltvdksrwqqgnvfscsvmhealhnhy |
| | tqkslslspg (SEQ ID NO: 1096) |
| TNT-336 | KVQLVESGGGLVQPGGSLRLSCEASGFTFSNYWMHWVRQVPGEGLVWVSRLN |
| | NGGTSTRYADSVKSRFTISRDNAKNTLYLQMNSLRAEDTAVYYCVRDWYFDLW |
| | GRGTLVTVSSastkgpsvfplapsskstsggtaalgclvkdyfpepvtvswnsgaltsgvhtfpavlqssglyslssvv |
| | tvpssslgtqtyicnvnhkpsntkvdkkvepkscdkthtcppcpapellggpsvflfppkpkdtlmisrtpevtcvvvdvsh |
| | edpevkfnwyvdgvevhnaktkpreeqynstyrvvsvltvlhqdwlngkeykckvsnkalpapiektiskakgqprepq |
| | vytlppsreemtknqvsltclvkgfypsdiavewesngqpennykttppvldsdgsfflyskltvdksrwqqgnvfscsvm |
| | healhnhytqkslslspg (SEQ ID NO: 1097) |
| TNT-337 | EVQLVESGGGLVQPGGSLRLSCAASGFTFSNYDMSWVRQAPGKGLEWVATISSG |
| | GSYTYYVDSVKGRFTISRDNAKNSLYLQMNSLRAEDTAVYYCARQELGSYYAM |
| | DYWGQGTTVTVSSastkgpsvfplapsskstsggtaalgclvkdyfpepvtvswnsgaltsgvhtfpavlqssgly |
| | slssvvtvpssslgtqtyicnvnhkpsntkvdkkvepkscdkthtcppcpapellggpsvflfppkpkdtlmisrtpevtcvv |
| | vdvshedpevkfnwyvdgvevhnaktkpreeqynstyrvvsvltvlhqdwlngkeykckvsnkalpapiektiskakgq |
| | prepqvytlppsreemtknqvsltclvkgfypsdiavewesngqpennykttppvldsdgsfflyskltvdksrwqqgnvf |
| | scsvmhealhnhytqkslslspg (SEQ ID NO: 1098) |
| TNT-412 | QVQLVQSGAEVKKPGASVKVSCKASGYTFTGYYMHWVRQAPGQGLEWMGWI |
| | NPNSGGTNYAQKFQGRVTMTRDTSISTAYMELSRLRSDDTAVYYCARGSALHFF |
| | GVFDYWGQGTLVTVSSastkgpsvfplapsskstsggtaalgclvkdyfpepvtvswnsgaltsgvhtfpavlq |
| | ssglyslssvvtvpssslgtqtyicnvnhkpsntkvdkkvepkscdkthtcppcpapellggpsvflfppkpkdtlmisrtpe |
| | vtcvvvdvshedpevkfnwyvdgvevhnaktkpreeqynstyrvvsvltvlhqdwlngkeykckvsnkalpapiektis |
| | kakgqprepqvytlppsreemtknqvsltclvkgfypsdiavewesngqpennykttppvldsdgsfflyskltvdksrwq |
| | qgnvfscsvmhealhnhytqkslslspg (SEQ ID NO: 1099) |
| TNT-417 | QVQLVQSGAEVKKPGSSVKVSCKASGGTFSSYAISWVRQAPGQGLEWMGGIIPIF |
| | GTANYAQKFQGRVTITADKSTSTAYMELSSLRSEDTAVYYCAAGYYYDSSGYY |
| | HQPYYFDYWGQGTLVTVSSastkgpsvfplapsskstsggtaalgclvkdyfpepvtvswnsgaltsgvhtfp |
| | avlqssglyslssvvtvpssslgtqtyicnvnhkpsntkvdkkvepkscdkthtcppcpapellggpsvflfppkpkdtlmis |
| | rtpevtcvvvdvshedpevkfnwyvdgvevhnaktkpreeqynstyrvvsvltvlhqdwlngkeykckvsnkalpapie |
| | ktiskakgqprepqvytlppsreemtknqvsltclvkgfypsdiavewesngqpennykttppvldsdgsfflyskltvdks |
| | rwqqgnvfscsvmhealhnhytqkslslspg (SEQ ID NO: 1100) |
| TNT-418 | QVQLQESGPGLVKPSETLSLTCTVSGGSISDYYWIWIRQPAGKGLEWIGRIYTTGG |
| | AKYNPSLKSRVTMSVDTSKNQFSLKLSSVTAADTAVYFCARDRNWEWNFDLWG |
| | RGTLVTVSSastkgpsvfplapsskstsggtaalgclvkdyfpepvtvswnsgaltsgvhtfpavlqssglyslssvvtv |
| | pssslgtqtyicnvnhkpsntkvdkkvepkscdkthtcppcpapellggpsvflfppkpkdtlmisrtpevtcvvvdvshed |
| | pevkfnwyvdgvevhnaktkpreeqynstyrvvsvltvlhqdwlngkeykckvsnkalpapiektiskakgqprepqvy |
| | tlppsreemtknqvsltclvkgfypsdiavewesngqpennykttppvldsdgsfflyskltvdksrwqqgnvfscsvmhe |
| | alhnhytqkslslspg (SEQ ID NO: 1101) |
| TNT-419 | QVQLQESGPGLVKPSETLSLTCTVSGGSISDYYWSWIRQPAGKGLEWIGRIYTTG |
| | GAKYNPSLKSRVTMSVDTSKNQFSLKLSSVTAADTAVYYCARDRNWEWNFDL |
| | WGRGTLVTVSSastkgpsvfplapsskstsggtaalgclvkdyfpepvtvswnsgaltsgvhtfpavlqssglyslss |
| | vvtvpssslgtqtyicnvnhkpsntkvdkkvepkscdkthtcppcpapellggpsvflfppkpkdtlmisrtpevtcvvvdv |
| | shedpevkfnwyvdgvevhnaktkpreeqynstyrvvsvltvlhqdwlngkeykckvsnkalpapiektiskakgqpre |
| | pqvytlppsreemtknqvsltclvkgfypsdiavewesngqpennykttppvldsdgsfflyskltvdksrwqqgnvfscs |
| | vmhealhnhytqkslslspg (SEQ ID NO: 1102) |
| TNT-420 | QVQLQESGPGLVKPSETLSLTCTVSGGSMSSYFWSWIRQPPGKGLEWIGYMYYS |
| | GNTNYNPSLKSRVTISVDTSKNQFSLKLSSVTAADTAVYYCARSGNFYWNFDLW |
| | GRGTLVTVSSastkgpsvfplapsskstsggtaalgclvkdyfpepvtvswnsgaltsgvhtfpavlqssglyslssvv |
| | tvpssslgtqtyicnvnhkpsntkvdkkvepkscdkthtcppcpapellggpsvflfppkpkdtlmisrtpevtcvvvdvsh |
| | edpevkfnwyvdgvevhnaktkpreeqynstyrvvsvltvlhqdwlngkeykckvsnkalpapiektiskakgqprepq |
| | vytlppsreemtknqvsltclvkgfypsdiavewesngqpennykttppvldsdgsfflyskltvdksrwqqgnvfscsvm |
| | healhnhytqkslslspg (SEQ ID NO: 1103) |
| TNT-421 | QVQLQESGPGLVKPSETLSLTCTVSGGSISSYYWSWIRQPPGKGLEWIGYIFYIGN |
| | TNYYPSLKSRVTISVDTSKNQFSLKLSSVTAADTAVYYCARSYPYWYFDLWGRG |
| | TLVTVSSastkgpsvfplapsskstsggtaalgclvkdyfpepvtvswnsgaltsgvhtfpavlqssglyslssvvtvpss |
| | slgtqtyicnvnhkpsntkvdkkvepkscdkthtcppcpapellggpsvflfppkpkdtlmisrtpevtcvvvdvshedpe |
| | vkfnwyvdgvevhnaktkpreeqynstyrvvsvltvlhqdwlngkeykckvsnkalpapiektiskakgqprepqvytlp |
| | psreemtknqvsltclvkgfypsdiavewesngqpennykttppvldsdgsfflyskltvdksrwqqgnvfscsvmheal |
| | hnhytqkslslspg (SEQ ID NO: 1104) |
| TNT-413 | EVQLVQSGAEVKKPGSSVKVSCKASGYTFTGYYMHWVRQAPGQGLEWMGWIN |
| | PNSGGTNYAQKFQGRVTMTRDTSISTAYMELRSLRSDDTAVYYCARGSALHFFG |
| | VFDYWGQGTLVTVSSastkgpsvfplapsskstsggtaalgclvkdyfpepvtvswnsgaltsgvhtfpavlqss |
| | glyslssvvtvpssslgtqtyicnvnhkpsntkvdkkvepkscdkthtcppcpapellAgpsvflfppkpkdtlmisrtpevt |
| | cvvvdvshedpevkfnwyvdgvevhnaktkpreeqynstyrvvsvltvlhqdwlngkeykckvsnkalpapiektiska |
| | kgqprepqvytlppsreemtknqvsltclvkgfypsdiavewesngqpennykttppvldsdgsfflyskltvdksrwqqg |
| | nvfscsvmhealhnhytqkslslspg (SEQ ID NO: 1105) |
| TNT-422 | EVQLVQSGAEVKKPGASVKVSCKASGYTFTSYDINWVRQAPGQGLEWMGGIIPI |
| | FGTANYAQKFQGRVTITADESTSTAYMELRSLRSEDTAVYYCASTAVAGFGIDY |
| | WGQGTLVTVSSastkgpsvfplapsskstsggtaalgclvkdyfpepvtvswnsgaltsgvhtfpavlqssglyslss |
| | vvtvpssslgtqtyicnvnhkpsntkvdkkvepkscdkthtcppcpapellAgpsvflfppkpkdtlmisrtpevtcvvvd |
| | vshedpevkfnwyvdgvevhnaktkpreeqynstyrvvsvltvlhqdwlngkeykckvsnkalpapiektiskakgqpr |
| | epqvytlppsreemtknqvsltclvkgfypsdiavewesngqpennykttppvldsdgsfflyskltvdksrwqqgnvfsc |
| | svmhealhnhytqkslslspg (SEQ ID NO: 1106) |
| TNT-423 | EVQLVQSGAEVKKPGSSVKVSCKASGGTFSSYAISWVRQAPGQGLEWMGGIIPIF |
| | GTANYAQKFQGRVTITADKSTSTAYMELSSLRSEDTAVYYCAAGYYYDSSGYY |
| | HQPYYFDYWGQGTLVTVSSastkgpsvfplapsskstsggtaalgclvkdyfpepvtvswnsgaltsgvhtfp |
| | avlqssglyslssvvtvpssslgtqtyicnvnhkpsntkvdkkvepkscdkthtcppcpapellAgpsvflfppkpkdtlmi |
| | srtpevtcvvvdvshedpevkfnwyvdgvevhnaktkpreeqynstyrvvsvltvlhqdwlngkeykckvsnkalpapi |
| | ektiskakgqprepqvytlppsreemtknqvsltclvkgfypsdiavewesngqpennykttppvldsdgsfflyskltvdk |
| | srwqqgnvfscsvmhealhnhytqkslslspg (SEQ ID NO: 1107) |
| TNT-424 | EVQLVQSGGGVVQPGRSLRLSCAASGFTFSSYAMHWVRQAPGKGLEWVAVISY |
| | DGSNKYYADSVKGRFTISRDNSKNTLYLQMNSLRAEDTAVYYCARATLQLWLP |
| | LFDYWGQGTLVTVSSastkgpsvfplapsskstsggtaalgclvkdyfpepvtvswnsgaltsgvhtfpavlqss |
| | glyslssvvtvpssslgtqtyicnvnhkpsntkvdkkvepkscdkthtcppcpapellAgpsvflfppkpkdtlmisrtpevt |
| | cvvvdvshedpevkfnwyvdgvevhnaktkpreeqynstyrvvsvltvlhqdwlngkeykckvsnkalpapiektiska |
| | kgqprepqvytlppsreemtknqvsltclvkgfypsdiavewesngqpennykttppvldsdgsfflyskltvdksrwqqg |
| | nvfscsvmhealhnhytqkslslspg (SEQ ID NO: 1108) |
| TNT-425 | QVQLQESGPGLVKPSETLSLTCTVSGGSISDYYWIWIRQPAGKGLEWIGRIYTTGG |
| | AKYNPSLKSRVTMSIDTSKNNFSLKLSSVTAADTAVYFCARDRNWEWNFDLWG |
| | RGTLVTVSSastkgpsvfplapsskstsggtaalgclvkdyfpepvtvswnsgaltsgvhtfpavlqssglyslssvvtv |
| | pssslgtqtyicnvnhkpsntkvdkkvepkscdkthtcppcpapellAgpsvflfppkpkdtlmisrtpevtcvvvdvshe |
| | dpevkfnwyvdgvevhnaktkpreeqynstyrvvsvltvlhqdwlngkeykckvsnkalpapiektiskakgqprepqv |
| | ytlppsreemtknqvsltclvkgfypsdiavewesngqpennykttppvldsdgsfflyskltvdksrwqqgnvfscsvmh |
| | ealhnhytqkslslspg (SEQ ID NO: 1109) |
| TNT-426 | QVQLQESGPGLVKPSETLSLTCTVSGGSMSSYFWSWIRQPPERGLEWVGYMYYS |
| | GNTNYNPSLKSRVTTSIDTSKNQFSLKLTSVTAADTAVYYCARSGNFYWNFDLW |
| | GRGTLVTVSSastkgpsvfplapsskstsggtaalgclvkdyfpepvtvswnsgaltsgvhtfpavlqssglyslssvv |
| | tvpssslgtqtyicnvnhkpsntkvdkkvepkscdkthtcppcpapellAgpsvflfppkpkdtlmisrtpevtcvvvdvsh |
| | edpevkfnwyvdgvevhnaktkpreeqynstyrvvsvltvlhqdwlngkeykckvsnkalpapiektiskakgqprepq |
| | vytlppsreemtknqvsltclvkgfypsdiavewesngqpennykttppvldsdgsfflyskltvdksrwqqgnvfscsvm |
| | healhnhytqkslslspg (SEQ ID NO: 1110) |
| TNT-427 | QVQLQESGPGLVKPSETLSLTCTISGGSISSYYWSWIRQPPGKGLEWIGYIFYIGNT |
| | NYYPSLKSRVTISVDASKNQFSLKLSSVTAADTAVYYCARSYPYWYFDLWGRGT |
| | LVTVSSastkgpsvfplapsskstsggtaalgclvkdyfpepvtvswnsgaltsgvhtfpavlqssglyslssvvtvpsssl |
| | gtqtyicnvnhkpsntkvdkkvepkscdkthtcppcpapellAgpsvflfppkpkdtlmisrtpevtcvvvdvshedpev |
| | kfnwyvdgvevhnaktkpreeqynstyrvvsvltvlhqdwlngkeykckvsnkalpapiektiskakgqprepqvytlpp |
| | sreemtknqvsltclvkgfypsdiavewesngqpennykttppvldsdgsfflyskltvdksrwqqgnvfscsvmhealh |
| | nhytqkslslspg (SEQ ID NO: 1111) |
| TNT-414 | QVQLVQSGAEVKKPGASVKVSCKASGYTFTGYYMHWVRQAPGQGLEWMGWI |
| | NPNSGGTNYAQKFQGRVTMTRDTSISTAYMELSRLRSDDTAVYYCARGSALHFF |
| | GVFDYWGQGTLVTVSSastkgpsvfplapsskstsggtaalgclvkdyfpepvtvswnsgaltsgvhtfpavlq |
| | ssglyslssvvtvpssslgtqtyicnvnhkpsntkvdkkvepkscdkthtcppcpapellAgpsvflfppkpkdtlmisrtpe |
| | vtcvvvdvshedpevkfnwyvdgvevhnaktkpreeqynstyrvvsvltvlhqdwlngkeykckvsnkalpapiektis |
| | kakgqprepqvytlppsreemtknqvsltclvkgfypsdiavewesngqpennykttppvldsdgsfflyskltvdksrwq |
| | qgnvfscsvmhealhnhytqkslslspg (SEQ ID NO: 1112) |
| TNT-428 | QVQLVQSGAEVKKPGSSVKVSCKASGGTFSSYAISWVRQAPGQGLEWMGGIIPIF |
| | GTANYAQKFQGRVTITADKSTSTAYMELSSLRSEDTAVYYCAAGYYYDSSGYY |
| | HQPYYFDYWGQGTLVTVSSastkgpsvfplapsskstsggtaalgclvkdyfpepvtvswnsgaltsgvhtfp |
| | avlqssglyslssvvtvpssslgtqtyicnvnhkpsntkvdkkvepkscdkthtcppcpapellAgpsvflfppkpkdtlmi |
| | srtpevtcvvvdvshedpevkfnwyvdgvevhnaktkpreeqynstyrvvsvltvlhqdwlngkeykckvsnkalpapi |
| | ektiskakgqprepqvytlppsreemtknqvsltclvkgfypsdiavewesngqpennykttppvldsdgsfflyskltvdk |
| | srwqqgnvfscsvmhealhnhytqkslslspg (SEQ ID NO: 1113) |
| TNT-429 | QVQLQESGPGLVKPSETLSLTCTVSGGSISDYYWIWIRQPAGKGLEWIGRIYTTGG |
| | AKYNPSLKSRVTMSVDTSKNQFSLKLSSVTAADTAVYFCARDRNWEWNFDLWG |
| | RGTLVTVSSastkgpsvfplapsskstsggtaalgclvkdyfpepvtvswnsgaltsgvhtfpavlqssglyslssvvtv |
| | pssslgtqtyicnvnhkpsntkvdkkvepkscdkthtcppcpapellAgpsvflfppkpkdtlmisrtpevtcvvvdvshe |
| | dpevkfnwyvdgvevhnaktkpreeqynstyrvvsvltvlhqdwlngkeykckvsnkalpapiektiskakgqprepqv |
| | ytlppsreemtknqvsltclvkgfypsdiavewesngqpennykttppvldsdgsfflyskltvdksrwqqgnvfscsvmh |
| | ealhnhytqkslslspg (SEQ ID NO: 1114) |
| TNT-430 | QVQLQESGPGLVKPSETLSLTCTVSGGSISDYYWSWIRQPAGKGLEWIGRIYTTG |
| | GAKYNPSLKSRVTMSVDTSKNQFSLKLSSVTAADTAVYYCARDRNWEWNFDL |
| | WGRGTLVTVSSastkgpsvfplapsskstsggtaalgclvkdyfpepvtvswnsgaltsgvhtfpavlqssglyslss |
| | vvtvpssslgtqtyicnvnhkpsntkvdkkvepkscdkthtcppcpapellAgpsvflfppkpkdtlmisrtpevtcvvvd |
| | vshedpevkfnwyvdgvevhnaktkpreeqynstyrvvsvltvlhqdwlngkeykckvsnkalpapiektiskakgqpr |
| | epqvytlppsreemtknqvsltclvkgfypsdiavewesngqpennykttppvldsdgsfflyskltvdksrwqqgnvfsc |
| | svmhealhnhytqkslslspg (SEQ ID NO: 1115) |
| TNT-431 | QVQLQESGPGLVKPSETLSLTCTVSGGSMSSYFWSWIRQPPGKGLEWIGYMYYS |
| | GNTNYNPSLKSRVTISVDTSKNQFSLKLSSVTAADTAVYYCARSGNFYWNFDLW |
| | GRGTLVTVSSastkgpsvfplapsskstsggtaalgclvkdyfpepvtvswnsgaltsgvhtfpavlqssglyslssvv |
| | tvpssslgtqtyicnvnhkpsntkvdkkvepkscdkthtcppcpapellAgpsvflfppkpkdtlmisrtpevtcvvvdvsh |
| | edpevkfnwyvdgvevhnaktkpreeqynstyrvvsvltvlhqdwlngkeykckvsnkalpapiektiskakgqprepq |
| | vytlppsreemtknqvsltclvkgfypsdiavewesngqpennykttppvldsdgsfflyskltvdksrwqqgnvfscsvm |
| | healhnhytqkslslspg (SEQ ID NO: 1116) |
| TNT-432 | QVQLQESGPGLVKPSETLSLTCTVSGGSISSYYWSWIRQPPGKGLEWIGYIFYIGN |
| | TNYYPSLKSRVTISVDTSKNQFSLKLSSVTAADTAVYYCARSYPYWYFDLWGRG |
| | TLVTVSSastkgpsvfplapsskstsggtaalgclvkdyfpepvtvswnsgaltsgvhtfpavlqssglyslssvvtvpss |
| | slgtqtyicnvnhkpsntkvdkkvepkscdkthtcppcpapellAgpsvflfppkpkdtlmisrtpevtcvvvdvshedpe |
| | vkfnwyvdgvevhnaktkpreeqynstyrvvsvltvlhqdwlngkeykckvsnkalpapiektiskakgqprepqvytlp |
| | psreemtknqvsltclvkgfypsdiavewesngqpennykttppvldsdgsfflyskltvdksrwqqgnvfscsvmheal |
| | hnhytqkslslspg (SEQ ID NO: 1117) |

In some embodiments, the nectin-4 antibody or conjugate comprises a light chain comprising SEQ ID NO:236 and a heavy chain comprising SEQ ID NO:224. In some embodiments, the nectin-4 antibody or conjugate comprises a light chain comprising SEQ ID NO:236 and a heavy chain comprising SEQ ID NO: 1070.

In some embodiments, the nectin-4 antibody or conjugate comprises a light chain comprising SEQ ID NO:237 and a heavy chain comprising SEQ ID NO:225. In some embodiments, the nectin-4 antibody or conjugate comprises a light chain comprising SEQ ID NO:237 and a heavy chain comprising SEQ ID NO:1071.

In some embodiments, the nectin-4 antibody or conjugate comprises a light chain comprising SEQ ID NO:238 and a heavy chain comprising SEQ ID NO:226. In some embodiments, the nectin-4 antibody or conjugate comprises a light chain comprising SEQ ID NO:238 and a heavy chain comprising SEQ ID NO:1072.

In some embodiments, the nectin-4 antibody or conjugate comprises a light chain comprising SEQ ID NO:239 and a heavy chain comprising SEQ ID NO:227. In some embodiments, the nectin-4 antibody or conjugate comprises a light chain comprising SEQ ID NO:239 and a heavy chain comprising SEQ ID NO:1073.

In some embodiments, the nectin-4 antibody or conjugate comprises a light chain comprising SEQ ID NO:240 and a heavy chain comprising SEQ ID NO:228. In some embodiments, the nectin-4 antibody or conjugate comprises a light chain comprising SEQ ID NO:240 and a heavy chain comprising SEQ ID NO:1074.

In some embodiments, the nectin-4 antibody or conjugate comprises a light chain comprising SEQ ID NO:241 and a heavy chain comprising SEQ ID NO:229. In some embodiments, the nectin-4 antibody or conjugate comprises a light chain comprising SEQ ID NO:241 and a heavy chain comprising SEQ ID NO:1075.

In some embodiments, the nectin-4 antibody or conjugate comprises a light chain comprising SEQ ID NO:242 and a heavy chain comprising SEQ ID NO:230. In some embodiments, the nectin-4 antibody or conjugate comprises a light chain comprising SEQ ID NO:242 and a heavy chain comprising SEQ ID NO:1076.

In some embodiments, the nectin-4 antibody or conjugate comprises a light chain comprising SEQ ID NO:243 and a heavy chain comprising SEQ ID NO:231. In some embodiments, the nectin-4 antibody or conjugate comprises a light chain comprising SEQ ID NO:243 and a heavy chain comprising SEQ ID NO:1077.

In some embodiments, the nectin-4 antibody or conjugate comprises a light chain comprising SEQ ID NO:244 and a heavy chain comprising SEQ ID NO:232. In some embodiments, the nectin-4 antibody or conjugate comprises a light chain comprising SEQ ID NO:244 and a heavy chain comprising SEQ ID NO:1078.

In some embodiments, the nectin-4 antibody or conjugate comprises a light chain comprising SEQ ID NO:245 and a heavy chain comprising SEQ ID NO:233. In some embodiments, the nectin-4 antibody or conjugate comprises a light chain comprising SEQ ID NO:245 and a heavy chain comprising SEQ ID NO:1079.

In some embodiments, the nectin-4 antibody or conjugate comprises a light chain comprising SEQ ID NO:246 and a heavy chain comprising SEQ ID NO:234. In some embodiments, the nectin-4 antibody or conjugate comprises a light chain comprising SEQ ID NO:246 and a heavy chain comprising SEQ ID NO:1080.

In some embodiments, the nectin-4 antibody or conjugate comprises a light chain comprising SEQ ID NO:247 and a heavy chain comprising SEQ ID NO:235. In some embodiments, the nectin-4 antibody or conjugate comprises a light chain comprising SEQ ID NO:247 and a heavy chain comprising SEQ ID NO:1081.

In some embodiments, the nectin-4 antibody or conjugate comprises a light chain comprising SEQ ID NO:1000 and a heavy chain comprising SEQ ID NO:1030. In some embodiments, the nectin-4 antibody or conjugate comprises a light chain comprising SEQ ID NO:1000 and a heavy chain comprising SEQ ID NO:1082.

In some embodiments, the nectin-4 antibody or conjugate comprises a light chain comprising SEQ ID NO:1001 and a heavy chain comprising SEQ ID NO:1031. In some embodiments, the nectin-4 antibody or conjugate comprises a light chain comprising SEQ ID NO:1001 and a heavy chain comprising SEQ ID NO:1083.

In some embodiments, the nectin-4 antibody or conjugate comprises a light chain comprising SEQ ID NO:1002 and a heavy chain comprising SEQ ID NO:1032. In some embodiments, the nectin-4 antibody or conjugate comprises a light chain comprising SEQ ID NO:1002 and a heavy chain comprising SEQ ID NO:1084.

In some embodiments, the nectin-4 antibody or conjugate comprises a light chain comprising SEQ ID NO:1003 and a heavy chain comprising SEQ ID NO:1033. In some embodiments, the nectin-4 antibody or conjugate comprises a light chain comprising SEQ ID NO:1003 and a heavy chain comprising SEQ ID NO:1085.

In some embodiments, the nectin-4 antibody or conjugate comprises a light chain comprising SEQ ID NO:1004 and a heavy chain comprising SEQ ID NO:1034. In some embodiments, the nectin-4 antibody or conjugate comprises a light chain comprising SEQ ID NO:1004 and a heavy chain comprising SEQ ID NO:1086.

In some embodiments, the nectin-4 antibody or conjugate comprises a light chain comprising SEQ ID NO:1005 and a heavy chain comprising SEQ ID NO:1035. In some embodiments, the nectin-4 antibody or conjugate comprises a light chain comprising SEQ ID NO:1005 and a heavy chain comprising SEQ ID NO:1087.

In some embodiments, the nectin-4 antibody or conjugate comprises a light chain comprising SEQ ID NO:1006 and a heavy chain comprising SEQ ID NO:1036. In some embodiments, the nectin-4 antibody or conjugate comprises a light chain comprising SEQ ID NO:1006 and a heavy chain comprising SEQ ID NO:1088.

In some embodiments, the nectin-4 antibody or conjugate comprises a light chain comprising SEQ ID NO:1007 and a heavy chain comprising SEQ ID NO:1037. In some embodiments, the nectin-4 antibody or conjugate comprises a light chain comprising SEQ ID NO:1007 and a heavy chain comprising SEQ ID NO:1089.

In some embodiments, the nectin-4 antibody or conjugate comprises a light chain comprising SEQ ID NO:1008 and a heavy chain comprising SEQ ID NO:1038. In some embodiments, the nectin-4 antibody or conjugate comprises a light chain comprising SEQ ID NO:1008 and a heavy chain comprising SEQ ID NO:1090.

In some embodiments, the nectin-4 antibody or conjugate comprises a light chain comprising SEQ ID NO:1009 and a heavy chain comprising SEQ ID NO:1039. In some embodiments, the nectin-4 antibody or conjugate comprises a light chain comprising SEQ ID NO:1009 and a heavy chain comprising SEQ ID NO:1091.

In some embodiments, the nectin-4 antibody or conjugate comprises a light chain comprising SEQ ID NO:1010 and a heavy chain comprising SEQ ID NO:1040. In some embodiments, the nectin-4 antibody or conjugate comprises a light chain comprising SEQ ID NO:1010 and a heavy chain comprising SEQ ID NO:1092.

In some embodiments, the nectin-4 antibody or conjugate comprises a light chain comprising SEQ ID NO:1011 and a heavy chain comprising SEQ ID NO:1041. In some embodiments, the nectin-4 antibody or conjugate comprises a light chain comprising SEQ ID NO:1011 and a heavy chain comprising SEQ ID NO:1093.

In some embodiments, the nectin-4 antibody or conjugate comprises a light chain comprising SEQ ID NO:1012 and a heavy chain comprising SEQ ID NO:1042. In some embodiments, the nectin-4 antibody or conjugate comprises a light chain comprising SEQ ID NO:1012 and a heavy chain comprising SEQ ID NO:1094.

In some embodiments, the nectin-4 antibody or conjugate comprises a light chain comprising SEQ ID NO:1013 and a heavy chain comprising SEQ ID NO:1043. In some embodiments, the nectin-4 antibody or conjugate comprises a light chain comprising SEQ ID NO:1013 and a heavy chain comprising SEQ ID NO:1095.

In some embodiments, the nectin-4 antibody or conjugate comprises a light chain comprising SEQ ID NO:1014 and a heavy chain comprising SEQ ID NO:1044. In some embodiments, the nectin-4 antibody or conjugate comprises a light chain comprising SEQ ID NO:1014 and a heavy chain comprising SEQ ID NO:1096.

In some embodiments, the nectin-4 antibody or conjugate comprises a light chain comprising SEQ ID NO:1015 and a heavy chain comprising SEQ ID NO:1045. In some embodiments, the nectin-4 antibody or conjugate comprises a light chain comprising SEQ ID NO:1015 and a heavy chain comprising SEQ ID NO:1097.

In some embodiments, the nectin-4 antibody or conjugate comprises a light chain comprising SEQ ID NO:1016 and a heavy chain comprising SEQ ID NO:1046. In some embodiments, the nectin-4 antibody or conjugate comprises a light chain comprising SEQ ID NO:1016 and a heavy chain comprising SEQ ID NO:1098.

In some embodiments, the nectin-4 antibody or conjugate comprises a light chain comprising SEQ ID NO:1017 and a heavy chain comprising SEQ ID NO:1047. In some embodiments, the nectin-4 antibody or conjugate comprises a light chain comprising SEQ ID NO:1017 and a heavy chain comprising SEQ ID NO:1099.

In some embodiments, the nectin-4 antibody or conjugate comprises a light chain comprising SEQ ID NO:1018 and a heavy chain comprising SEQ ID NO:1048. In some embodiments, the nectin-4 antibody or conjugate comprises a light chain comprising SEQ ID NO:1018 and a heavy chain comprising SEQ ID NO:1100.

In some embodiments, the nectin-4 antibody or conjugate comprises a light chain comprising SEQ ID NO:1019 and a heavy chain comprising SEQ ID NO:1049. In some embodiments, the nectin-4 antibody or conjugate comprises a light chain comprising SEQ ID NO:1019 and a heavy chain comprising SEQ ID NO:1101.

In some embodiments, the nectin-4 antibody or conjugate comprises a light chain comprising SEQ ID NO:1020 and a heavy chain comprising SEQ ID NO:1050. In some embodiments, the nectin-4 antibody or conjugate comprises a light chain comprising SEQ ID NO:1020 and a heavy chain comprising SEQ ID NO:1102.

In some embodiments, the nectin-4 antibody or conjugate comprises a light chain comprising SEQ ID NO:1021 and a heavy chain comprising SEQ ID NO:1051. In some embodiments, the nectin-4 antibody or conjugate comprises a light chain comprising SEQ ID NO:1021 and a heavy chain comprising SEQ ID NO:1103.

In some embodiments, the nectin-4 antibody or conjugate comprises a light chain comprising SEQ ID NO:1022 and a heavy chain comprising SEQ ID NO:1052. In some embodiments, the nectin-4 antibody or conjugate comprises a light chain comprising SEQ ID NO:1022 and a heavy chain comprising SEQ ID NO:1104.

In some embodiments, the nectin-4 antibody or conjugate comprises a light chain comprising SEQ ID NO:236 and a heavy chain comprising SEQ ID NO:1053. In some embodiments, the nectin-4 antibody or conjugate comprises a light chain comprising SEQ ID NO:236 and a heavy chain comprising SEQ ID NO:1105.

In some embodiments, the nectin-4 antibody or conjugate comprises a light chain comprising SEQ ID NO:242 and a heavy chain comprising SEQ ID NO:1054. In some embodiments, the nectin-4 antibody or conjugate comprises a light chain comprising SEQ ID NO:242 and a heavy chain comprising SEQ ID NO:1106.

In some embodiments, the nectin-4 antibody or conjugate comprises a light chain comprising SEQ ID NO:243 and a heavy chain comprising SEQ ID NO:1055. In some embodiments, the nectin-4 antibody or conjugate comprises a light chain comprising SEQ ID NO:243 and a heavy chain comprising SEQ ID NO:1107.

In some embodiments, the nectin-4 antibody or conjugate comprises a light chain comprising SEQ ID NO:245 and a heavy chain comprising SEQ ID NO:1056. In some embodiments, the nectin-4 antibody or conjugate comprises a light chain comprising SEQ ID NO:245 and a heavy chain comprising SEQ ID NO:1108.

In some embodiments, the nectin-4 antibody or conjugate comprises a light chain comprising SEQ ID NO:1007 and a heavy chain comprising SEQ ID NO:1057. In some embodiments, the nectin-4 antibody or conjugate comprises a light chain comprising SEQ ID NO:1007 and a heavy chain comprising SEQ ID NO:1109.

In some embodiments, the nectin-4 antibody or conjugate comprises a light chain comprising SEQ ID NO:1008 and a heavy chain comprising SEQ ID NO:1058. In some embodiments, the nectin-4 antibody or conjugate comprises a light chain comprising SEQ ID NO:1008 and a heavy chain comprising SEQ ID NO:1110.

In some embodiments, the nectin-4 antibody or conjugate comprises a light chain comprising SEQ ID NO:1012 and a heavy chain comprising SEQ ID NO:1059. In some embodiments, the nectin-4 antibody or conjugate comprises a light chain comprising SEQ ID NO:1012 and a heavy chain comprising SEQ ID NO:1111.

In some embodiments, the nectin-4 antibody or conjugate comprises a light chain comprising SEQ ID NO:236 and a heavy chain comprising SEQ ID NO:1060. In some embodiments, the nectin-4 antibody or conjugate comprises a light chain comprising SEQ ID NO:1017 and a heavy chain comprising SEQ ID NO:1112.

In some embodiments, the nectin-4 antibody or conjugate comprises a light chain comprising SEQ ID NO:243 and a heavy chain comprising SEQ ID NO:1061. In some embodiments, the nectin-4 antibody or conjugate comprises a light chain comprising SEQ ID NO:1018 and a heavy chain comprising SEQ ID NO:1113.

In some embodiments, the nectin-4 antibody or conjugate comprises a light chain comprising SEQ ID NO:1007 and a heavy chain comprising SEQ ID NO:1062. In some embodiments, the nectin-4 antibody or conjugate comprises a light chain comprising SEQ ID NO:1019 and a heavy chain comprising SEQ ID NO:1114.

In some embodiments, the nectin-4 antibody or conjugate comprises a light chain comprising SEQ ID NO:1007 and a heavy chain comprising SEQ ID NO:1063. In some embodiments, the nectin-4 antibody or conjugate comprises a light chain comprising SEQ ID NO:1020 and a heavy chain comprising SEQ ID NO:1115.

In some embodiments, the nectin-4 antibody or conjugate comprises a light chain comprising SEQ ID NO:1008 and a heavy chain comprising SEQ ID NO:1064. In some embodiments, the nectin-4 antibody or conjugate comprises a light chain comprising SEQ ID NO:1021 and a heavy chain comprising SEQ ID NO:1116.

In some embodiments, the nectin-4 antibody or conjugate comprises a light chain comprising SEQ ID NO:1012 and a heavy chain comprising SEQ ID NO:1065. In some embodiments, the nectin-4 antibody or conjugate comprises a light chain comprising SEQ ID NO:1022 and a heavy chain comprising SEQ ID NO:1117.

Antibodies that target cell surface antigens can trigger immunostimulatory and effector functions that are associated with Fc receptor (FcR) engagement on immune cells. There are a number of Fc receptors that are specific for particular classes of antibodies, including IgG (gamma receptors), IgE (eta receptors), IgA (alpha receptors) and IgM (mu receptors). Binding of the Fc region to Fc receptors on cell surfaces can trigger a number of biological responses including phagocytosis of antibody-coated particles (antibody-dependent cell-mediated phagocytosis, or ADCP), clearance of immune complexes, lysis of antibody-coated cells by killer cells (antibody-dependent cell-mediated cytotoxicity, or ADCC) and, release of inflammatory mediators, placental transfer, and control of immunoglobulin production. Additionally, binding of the C₁ component of complement to antibodies can activate the complement system and complement dependent cytolysis (CDC). Activation of complement can be important for the lysis of cellular pathogens. However, the activation of complement can also stimulate the inflammatory response and can also be involved in autoimmune hypersensitivity or other immunological disorders. Variant Fc regions with reduced or ablated ability to bind certain Fc receptors are useful for developing therapeutic antibodies and Fc-fusion polypeptide constructs which act by targeting, activating, or neutralizing ligand functions while not damaging or destroying local cells or tissues. Variant Fc regions with enhanced or active Fc domains that bind to certain Fc receptors are also useful for developing therapeutic antibodies and Fc-fusion polypeptide constructs, and can provide an active or enhanced immune response. The Fc domain is not involved directly in binding an antibody to its target, but can be involved in various effector functions, such as participation of the antibody in ADCP, ADCC and CDC. In some embodiments, which may be combined with any of the foregoing embodiments, the nectin-4 antibody or nectin-4 antibody conjugate comprises an Fc region. In some embodiments of the nectin-4 antibodies and nectin-4 antibody conjugates described herein, the Fc region is a human IgG1 Fc region. In some instances, a human IgG1 Fc domain is a wildtype IgG1 Fc domain, such as human wildtype IgG1 Fc domain. In some instances, a human IgG1 Fc domain is a human IgG1 Fc variant, such as a human IgG1 Fc variant. In some instances, a human IgG1 Fc variant has up to 12, 11, 10, 9, 8, 7, 6, 5 or 4 or fewer mutations in total as compared to wild-type human IgG1 sequence. In some embodiments, one or more additional deletions are included in such IgG1 Fc variant. For example, in some embodiments, the C-terminal lysine of the Fc IgG1 heavy chain constant region is deleted, for example to increase the homogeneity of the polypeptide when the polypeptide is produced in bacterial or mammalian cells.

In some embodiments of the nectin-4 antibodies and nectin-4 antibody conjugates described herein, the Fc region is a variant Fc region (also referred to herein as non-native Fc regions and variant Fc domains) with reduced or ablated ability to bind certain Fc receptors. Such Fc variants are useful for developing therapeutic antibodies and therapeutic conjugates which act by targeting, activating, or neutralizing ligand functions while not damaging or destroying local cells or tissues. In some embodiments, the Fc domain in an antibody or conjugate of the disclosure comprises one or more amino acid substitutions, additions or insertions, deletions, or any combinations thereof that lead to decreased effector function such as decreased antibody-dependent cell-mediated cytotoxicity (ADCC), decreased complement-dependent cytolysis (CDC), decreased antibody-dependent cell-mediated phagocytosis (ADCP), or any combinations thereof. In some embodiments, the antibodies or conjugates of the disclosure are characterized by decreased binding (e.g., minimal binding or absence of binding) to a human Fc receptor and decreased binding (e.g., minimal binding or absence of binding) to complement protein C1q. In some embodiments, the antibodies or conjugates comprise an Fc region with reduced effector function and the Fc region comprises one or more of the mutations shown in Table 25, below.

TABLE 25
Fc modifications that reduced effector function.

| | | Reduced |
|---|---|---|
| | | effector |
| Mutation | Phenotype | function |
| Leu235Glu | Decreased binding to | ADCC |
| | cell surface FcγRs | |
| Leu234Ala/Leu235Ala | Decreased binding to | ADCC, |
| | FcγRI, II, III | ADCP, CDC |
| Ser228Pro/Leu235Glu | Decreased FcγRI binding | ADCC |
| Leu234Ala/Leu235Ala/ | Eliminated binding to | ADCP |
| Pro329Gly | FcγRI, II, III, C1q | |
| Leu234Glu/Leu235Phe/ | Decreased binding to | CDC |
| Pro331Ser | FcγRI, II, III and C1q | |
| Asp265Ala | Decreased binding to | ADCC |
| | FcγRI, II, III | ADCP |
| Gly237Ala | Decreased binding to FcγRII | ADCP |
| Glu318Ala | Decreased binding to FcγRII | ADCP |
| Glu233Pro | Decreased binding to | ADCC |
| | FcγRI, II, and III | ADCP |
| Gly236Arg/Leu328Arg | Decreased binding to all FcγR | ADCC |
| His268Gln/Val309Leu/ | Decreased binding to all FcγR | ADCC, |
| Ala330Ser/Pro331Ser | Decreased C1q binding | ADCP, CDC |
| Val234Ala/Gly237Ala/ | Near complete elimination of | ADCC, |
| Pro238Ser/His268Ala/ | FcγRI, IIa, IIb, | ADCP, CDC |
| Val309Leu/Ala330Ser/ | and IIIa binding | |
| Pro331Ser | Decreased C1q binding | |
| | Binds FcγRn | |
| Leu234Ala/L235Ala/ | Near complete elimination | ADCC, CDC |
| Gly237Ala/P238Ser/ | of FcγRI, IIa, | |
| His268Ala/Ala330Ser/ | IIb, and IIIa binding | |
| Pro331Ser | Binds FcγRn | |
| Leu235Ala/Gly237Ala/ | Unable to bind to human cell | ADCC |
| Glu318Ala | lines expressing FcγRs | |
| Ala330Leu | Decreased C1q binding | CDC |
| Asp270Ala | Decreased C1q binding | CDC |
| Lys322Ala | Decreased C1q binding | CDC |
| Pro329Ala | Decreased C1q binding | CDC |
| Pro331Ala | Decreased C1q binding | CDC |
| Val264Ala | Decreased C1q binding | CDC |
| Phe241Ala | Decreased C1q binding | CDC |
| Asn297Ala or | Decreased binding to | ADCC, |
| Gly or Gln | FcγRI and IIIa | ADCP, CDC |
| | Decreased C1q binding | |
| Ser228Pro/Phe234Ala/ | Decreased binding to | ADCC, CDC |
| Leu235Ala | FcγRI, IIa and IIIa | |
| Ser298Gly/Thr299Ala | Abolished or significantly | ADCC, CDC |
| | reduced binding to | |
| | C1q and most FcγRs | |
| Leu234Phe/Leu235Glu/ | No detectable binding | ADCC, CDC |
| Asp265Ala | to FcγRI and reduced | |
| | binding to C1q | |
| Ser267Glu/Leu328Phe | Increase in binding affinity | ADCC |
| | to FcγRIIB; eliminated | |
| | binding to FcγRIIIA-V158 | |
| Pro238Asp | Increase in binding affinity | ADCP |
| | to FcγRIIB; Reducing | |
| | binding to FcγRI, FcγRIIA- | |
| | H131, FcγRIIIA-V131 | |
| Glu237Asp/Pro238Asp/ | Increase in binding affinity | ADCP |
| His268Asp/Pro271Gly/ | to FcγRIIB | |
| Ala330Arg | | |
| Glu233Asp/Glu237Asp/ | Increase in binding affinity | ADCP |
| Pro238Asp/His268Asp/ | to FcγRIIB; Reduction | |
| Pro271Gly/Ala330Arg | in binding to FcγRI and | |
| | FcγRIIA-H131 | |
| Asn298Ala | Lacks Fc receptor and | ADCC, CDC |
| | complement binding | |

In some embodiments, an Fc domain can refer to a dimer of two Fc domain PP38T, monomers. In a wild-type Fc domain, two Fc domain monomers dimerize by the interaction between the two CH3 antibody constant domains, as well as one or more disulfide bonds that form between the hinge domains of the two dimerized Fc domain monomers. In some embodiments, an Fc domain is mutated to lack effector functions, for example a “dead Fc domain.” In some embodiments, each of the Fc domain monomers in an Fc domain includes amino acid substitutions in the CH₂ antibody constant domain to reduce the interaction or binding between the Fc domain and an Fc receptor, such as an Fcγ receptor (FcγR), an Fcα receptor (FcαR), or an Fcε (FcεR).

Antibody-dependent cell-mediated cytotoxicity, which is also referred to herein as ADCC, refers to a form of cytotoxicity in which secreted Ig bound onto Fc receptors (FcRs) present on certain cytotoxic cells (e.g., Natural Killer (NK) cells and neutrophils) enabling these cytotoxic effector cells to bind specifically to an antigen-bearing target cell and subsequently kill the target cell. Antibody-dependent cell-mediated phagocytosis, which is also referred to herein as ADCP, refers to a form of cytotoxicity in which secreted Ig bound onto Fc receptors (FcRs) present on certain phagocytic cells (e.g., macrophages) enabling these phagocytic effector cells to bind specifically to an antigen-bearing target cell and subsequently engulf and digest the target cell. Ligand-specific high-affinity IgG antibodies directed to the surface of target cells can stimulate the cytotoxic or phagocytic cells and can be used for such killing. Fc receptors include, but are not limited to, FcγRI (CD64), FcγRIIa (CD32a), FcγRIIb (CD32b), FcγRIIc (CD32c), FcγRIIIa (CD16a), and FcγRIIIb (CD16b).

In some embodiments, the IgG Fc variants herein are minimally glycosylated or have reduced glycosylation relative to a wild-type sequence. In some embodiments, the reduced glycosylation modulates the level of effector function provided by the Fc region. In some embodiments, deglycosylation is accomplished with a mutation of N297A, or by mutating N297 to any amino acid which is not N. In some embodiments, deglycosylation is accomplished by disrupting the motif N-Xaa1-Xaa2-Xaa3 (wherein N=asparagine; Xaa1=any amino acid except P (proline); Xaa2=T (threonine), S (serine) or C (cysteine); and Xaa3=any amino acid except P (proline)). In one embodiment, the N-Xaa1-Xaa2-Xaa3 motif refers to residues 297-300 as designated according to Kabat et al., 1991. In some embodiments, a mutation to any one or more of N, Xaa1, Xaa2, or Xaa3 results in deglycosylation of the Fc variant.

In some embodiments, the antibodies or conjugates herein comprising a variant Fc region exhibit reduced or ablated binding to at least one of Fcγ receptors CD16a, CD32a, CD32b, CD32c, and CD64 as compared to a polypeptide construct comprising a native Fc region. In some cases, the antibodies or conjugates described herein exhibit reduced or ablated binding to CD16a, CD32a, CD32b, CD32c, and CD64 Fcγ receptors. In some embodiments, the Fc variant exhibits reduced binding to an Fc receptor of the subject compared to the wild-type human IgG Fc region. In some embodiments, the Fc variant exhibits ablated binding to an Fc receptor of the subject compared to the wild-type human IgG Fc region. In some instances, an Fc variant has reduced binding to an Fcγ receptor by a factor of 10%, 20% 30%, 40%, 50%, 60%, 70%, 80%, 90%, 95%, 96%, 97%, 98%, 99% or 100% (fully ablated effector function). In some embodiments, the reduced binding is for any one or more Fcγ receptor, e.g., CD16a, CD32a, CD32b, CD32c, or CD64. In some embodiments, the reduction or ablation in binding to an Fc7 receptor results in a reduction or ablation of ADCC. In some embodiments, antibodies or conjugates comprising an Fc variant as described herein exhibit at least a 5%, 10%, 15%, 20%, 30%, 40%, 50%, 60%, 70%, 80%, 90% or greater reduction in ADCC. In some embodiments, antibodies or conjugates comprising an Fc variant as described herein exhibit a 100% (fully ablated) ADCC.

In some embodiments, the Fc variant exhibits a reduction of phagocytosis (ADCP) compared to the wild-type human IgG Fc region. Such Fc variants exhibit a reduction in phagocytosis compared to its wild-type human IgG Fc region, wherein the reduction of phagocytosis activity is e.g., by a factor of 10%, 20%, 30%, 40%, 50%, 60%, 70%, 80%, 90%, 95%, 96%, 97%, 98%, 99% or 100%. In some instances, an Fc variant exhibits ablated phagocytosis compared to its wild-type human IgG Fc region.

Complement-directed cytotoxicity, which is also referred to herein as CDC, refers to a form of cytotoxicity in which the complement cascade is activated by the complement component C1q binding to antibody Fc. In some embodiments, the antibodies and conjugates comprising a non-native Fc region described herein exhibit at least a 5%, 10%, 15%, 20%, 30%, 40%, 50%, 60%, 70%, 80%, 90% or greater reduction in C1q binding compared to the antibody or conjugate comprising a wild-type Fc region. In some cases, antibodies or conjugates comprising a non-native Fc region as described herein exhibit reduced CDC as compared to antibodies or conjugates comprising a wild-type Fc region. In some embodiments, antibodies or conjugates comprising a non-native Fc region as described herein exhibit at least a 5%, 10%, 15%, 20%, 30%, 40%, 50%, 60%, 70%, 80%, 90% or greater reduction in CDC compared to antibodies or conjugates comprising a wild-type Fc region. In some cases, antibodies or conjugates comprising a non-natural Fc variant as described herein exhibit negligible CDC as compared to antibodies or conjugates comprising a wild-type Fc region.

In some cases, antibodies or conjugates comprising a non-natural Fc variant as described herein exhibit reduced or negligible CDC and also are characterized by decreased binding (e.g., minimal binding or absence of binding) to one or more of human FcγRI (CD64), FcγRIIa (CD32a), FcγRIIb (CD32b), FcγRIIc (CD32c), FcγRIIIa (CD16a), and FcγRIIIb (CD16b). In some embodiments, the Fc region of the antibodies or conjugates described herein has wild-type or native CDC activity. In some embodiments, the Fc region of the antibodies or conjugates described herein has wild-type or native CDC activity and is characterized by decreased binding (e.g., minimal binding or absence of binding) to one or more of human FcγRI (CD64), FcγRIIa (CD32a), FcγRIIb (CD32b), FcγRIIc (CD32c), FcγRIIIa (CD16a), and FcγRIIIb (CD16b).

In certain embodiments, the nectin-4 antibodies or nectin-4 antibody conjugates described herein comprise an IgG Fc region selected from the group consisting of an IgG1 Fc region, an IgG2 Fc region, and an IgG4 Fc region. In some embodiments, the Fc region is a wild-type human IgG1, IgG2, or IgG4 Fc region. In some embodiments, the IgG Fc region is a human IgG Fc region comprising one or more amino acid substitutions that reduce or eliminate one or more effector functions, as compared with the effector function(s) of a human Fc region that lacks the amino acid substitution(s). For example, in some embodiments, an IgG Fc domain is mutated to lack effector functions, typical of a “dead” Fc domain. In some embodiments, to alter or reduce an antibody-dependent effector function, such as ADCC, CDC, ADCP, or any combinations thereof, the Fc domains in antibodies or conjugates of the disclosure are of the IgG class and comprise one or more amino acid substitutions at E233, L234, L235, G236, G237, D265, D270, N297, E318, K320, K322, A327, A330, P331, or P329 (numbering according to the EU index of Kabat (Sequences of Proteins of Immunological Interest, 5th Ed. Public Health Service, National Institutes of Health, Bethesda, MD. (1991)); the Kabat numbering of amino acid residues can be determined for a given antibody by alignment at regions of homology of the sequence of the antibody with a “standard” Kabat numbered sequence).

In some embodiments, the IgG Fc variants herein are minimally glycosylated or have reduced glycosylation relative to a wild-type sequence. In some embodiments, the reduced glycosylation modulates the level of effector function provided by the Fc region. In some embodiments, deglycosylation is accomplished with a mutation of N297A, or by mutating N297 to any amino acid which is not N. In some embodiments, deglycosylation is accomplished by disrupting the motif N-Xaa1-Xaa2-Xaa3 (wherein N=asparagine; Xaa1=any amino acid except P (proline); Xaa2=T (threonine), S (serine) or C (cysteine); and Xaa3=any amino acid except P (proline)). In one embodiment, the N-Xaa1-Xaa2-Xaa3 motif refers to residues 297-300 as designated according to Kabat et al., 1991. In some embodiments, a mutation to any one or more of N, Xaa1, Xaa2, or Xaa3 results in deglycosylation of the Fc variant.

In some embodiments of the nectin-4 antibodies and nectin-4 antibody conjugates described herein, the Fc region is a human IgG1 Fc region. In some instances, a human IgG1 Fc domain is a wildtype IgG1 Fc domain, such as human wildtype IgG1 Fc domain. In some instances, a human IgG1 Fc domain is a human IgG1 Fc variant, such as a human IgG1 Fc variant. In some instances, a human IgG1 Fc variant has up to 12, 11, 10, 9, 8, 7, 6, 5 or 4 or fewer mutations in total as compared to wild-type human IgG1 sequence. In some embodiments, one or more additional deletions are included in such IgG1 Fc variant. For example, in some embodiments, the C-terminal lysine of the Fc IgG1 heavy chain constant region is deleted, for example to increase the homogeneity of the polypeptide when the polypeptide is produced in bacterial or mammalian cells.

In some embodiments, an Fc domain monomer is from an IgG1 antibody and includes one or more of amino acid substitutions L234A, L235A, G237A, and N297A (as designated according to the EU numbering system per Kabat et al., 1991). In some embodiments, one or more additional mutations are included in such IgG1 Fc variant. Non-limiting examples of such additional mutations for human IgG1 Fc variants include E318A and K322A.

In some embodiments, an Fc domain includes specific amino acid substitutions that are known to minimize the interaction between the Fc domain and an Fcγ receptor. In some instances, the Fc region is a human IgG1 Fc region comprising L234A, L235A, and/or G237A substitutions, amino acid position numbering according to EU index. In some embodiments, the Fc variant includes at least one of the mutations L234A, L235A, G237A In some embodiments, the Fc variant includes at least two of the mutations L234A, L235A, G237A of an IgG1 Fc region Fc region. In some embodiments, the Fc variant includes the mutations L234A, L235A, and G237A of an IgG1 Fc region (IgG1 AAA). In some embodiments, the IgG1 Fc variant includes 1, 2 or 3 of the mutations L234A, L235A, and G237A and further includes an additional one or more mutations such as N297A and D265A.

In some embodiments, the antibodies or conjugates comprise a variant Fc region with one or modifications that improve antibody half-life, for example by association of the antibody with the neonatal Fc receptor (FcRn) leading to antibody recycling and minimal endosomal degradation. In some cases, Fc variants with extended half-life may have increased or enhanced binding to or affinity for one or more Fc receptors. In some cases, the binding or affinity may be affected at a specific pH range, for example related to the pH in the endosome. In some embodiments, the antibodies or conjugates herein comprise a variant Fc region with one or modifications that improve antibody circulation half-life and comprise one or more mutations provided in Table 26, below.

TABLE 26
Fc modifications to improve antibody circulation half-life.

| Mutation | Phenotype |
|---|---|
| Arg435His | Increased binding to FcRn at low pH |
| Asn434Ala | Increased binding to FcRn at low pH |
| Met252Tyr/Ser254Thr/ | Slowed off-rate for Fc and FcRn |
| Thr256Glu | Increased FcRn affinity |
| | Decreased ADCC |
| Met252Tyr/Ser254Thr/ | Increase in binding to FcRn at both |
| Thr256Glu + His433K/ | endosomal pH and extracellular pH |
| Asn434Phe | |
| Met428Leu/Asn434Ser | Increased affinity to and slowed off-rate |
| | for FcRn at pH |
| | No change in ADCC |
| Thr252Leu/Thr253Ser/ | Increased binding to FcRn at low pH |
| Thr254Phe | |
| Glu294delta/Thr307Pro/ | Increased binding to FcRn at low pH |
| Asn434Tyr | No binding to FcRn at extracellular pH |
| | Decreased FcgRIIa binding and ADCC |
| Thr256Asn/Ala378Val/ | Increased binding to FcRn at low pH |
| Ser383Asn/Asn434Tyr | No binding to FcRn at extracellular pH |
| Glu294delta | Increased sialylation |
| Thr252Leu/Thr254Ser/ | Increase biological half-life |
| Thr256Phe | |
| Met252Tyr/Val308Pro/ | Enhance binding to FcRn at endosomal |
| Asn343Tyr | pH and extracellular pH |
| Leu309Asp/Gln311His/ | Enhancement in binding to |
| Asn434Ser | FcRn at endosomal pH and no |
| | binding to FcRn at extracellular pH |
| His285Asp/Thr307Gln/ | Enhanced binding to FcRn at endosomal |
| Ala378Val | pH |
| His433Lys/Asn434Phe | Enhanced binding to FcRn at endosomal pH |
| | while minimizing changes to FcRn binding at |
| | extracellular pH |
| Val308Pro | Enhancement of binding to FcRn at endosomal |
| | pH, with no change in binding to FcRn at |
| | extracellular pH 7 |
| Thr250Gln/Met428Leu | Increase in binding to FcRn at endosomal pH |
| | while not affecting binding to FcRn at |
| | extracellular pH |
| Thr307Ala/Glu380Ala/ | Increase in FcRn binding at endosomal pH |
| Asn434Ala | while retaining equivalent binding to the |
| | low affinity FcγRs; increased binding to |
| | FcRn at extracellular pH |

In some embodiments of the nectin-4 antibodies and nectin-4 antibody conjugates described herein, the Fc region is a variant Fc region with enhanced effector function, such as enhanced binding or activity with respect to one or more Fc receptors. In some embodiments, the Fc region of the antibodies or conjugates described herein is characterized by enhanced or increased binding to one or more of human FcγRI (CD64), FcγRIIa (CD32a), FcγRIIb (CD32b), FcγRIIc (CD32c), FcγRIIIa (CD16a), and FcγRIIIb. In some cases, the variant Fc region has improved binding to C1q. In some embodiments, the Fc region of the antibodies or conjugates described herein is characterized by enhanced or increased binding to one or more of human FcγRI (CD64), FcγRIIa (CD32a), FcγRIIb (CD32b), FcγRIIc (CD32c), FcγRIIIa (CD16a), and FcγRIIIb and the Fc region has improved binding to C1q. In some embodiments, the antibodies or conjugates comprise an Fc region with enhanced effector function and the Fc region comprises one or more of the mutations shown in Table 27, below.

In some embodiments, the Fc region has been engineered to improve effector function, including, but not limited to, the Fc modifications and enhanced effector functions of Table 27, below. In some embodiments, the antibodies or conjugates herein comprise a variant Fc region exhibiting enhanced antibody-dependent cell-mediated cytotoxicity (ADCC) activity. In some embodiments, the variant Fc region exhibits at least a 5%, 10%, 15%, 20%, 30%, 40%, 50%, 60%, 70%, 80%, 90%, or greater increase in ADCC activity compared to the wild-type Fc region. In some embodiments, the antibodies or conjugates herein comprise a variant Fc region exhibiting enhanced antibody-dependent cellular phagocytosis (ADCP) activity. In some embodiments, the variant Fc region exhibits at least a 5%, 10%, 15%, 20%, 30%, 40%, 50%, 60%, 70%, 80%, 90%, or greater increase in ADCP activity compared to the wild-type Fc region. In some embodiments, the antibodies or conjugates herein comprise a variant Fc region exhibiting enhanced complement-dependent cytotoxicity (CDC) activity. In some embodiments, the variant Fc region exhibits at least a 5%, 10%, 15%, 20%, 30%, 40%, 50%, 60%, 70%, 80%, 90%, or greater increase in CDC activity compared to the wild-type Fc region.

TABLE 27
Fc modifications that enhance effector function.

| | | Enhanced |
|---|---|---|
| | | effector |
| Mutation | Phenotype | function |
| Ser298Ala/Glu333Ala/Lys334Ala | Enhanced FcγRIIIa affinity | ADCC |
| Ser239Asp/Ala330Leu/Ile332Glu | Increased FcγRIIIa affinity | ADCC, |
| | Low binding to inhibitory | ADCP |
| | FcγRIIb | |
| Ser239Asp/Ile332Glu | Increased FcγRIIIa | ADCC, |
| | Strong binding to inhibitory | ADCP |
| | FcγRIIb | |
| Gly236Ala/Ser239Asp/ | Increased binding affinity | ADCC |
| Ala330Leu/Ile332Glu | to FcγRIIa and FcγRIIIa | |
| Gly236Ala | Increases FcγRIIa affinity | ADCP |
| Gly236Ala/Ser239Asp/ | Increases FcγRIIIa and | ADCC, |
| Ile332Glu | FcγRIIa | ADCP |
| | Enhanced FcγRIIb binding | |
| Leu234Tyr/Gly236Trp/ | Improved FcγRIIIa affinity | ADCC |
| Ser298Ala | when present in 1 heavy | |
| | chain constant region | |
| Phe243Leu/Arg292Pro/ | Enhanced FcγRIIa and | ADCC |
| Tyr300Leu/Val305Ile/ | FcγRIIIa off-rates | |
| Pro396Leu | | |
| Ser267Glu/His268Phe/ | Increased C1q binding | ADCC, |
| Ser324Thr/Gly236Ala/ | | CDC |
| Ise332Glu | | |
| Glu345 Lys | Increased C1q binding | ADCC, |
| | | CDC |
| Glu430Gly | Increased C1q binding | ADCC, |
| | | CDC |
| Leu235Val/ | Increased FcγRIIIa binding | ADCC |
| Phe243Leu/Arg292Pro/ | | |
| Tyr300Leu/Pro396Leu | | |
| Glu236Ala/Ala330Leu/ | Enhanced binding to | ADCC |
| Ile332Glu | FcγRIIA and FcγRIIIA | |
| One heavy chain: | Increased binding affinity | ADCC |
| Leu234Tyr/Leu235Gln/ | to FcγRIIA-H131, | |
| Gly236Trp/Ser239Met/ | FcγRIIA-R131, FcγRIIIA- | |
| His268Asp/Asp270Glu/ | F158, FcγRIIIA-V158 | |
| Ser298Ala | | |
| opposing heavy chain: | | |
| Asp270Glu/Lys326Asp/ | | |
| Ala330Met/Lys334Glu | | |
| Lys326Trp/Glu333Ser | Increased C1q binding | CDC |
| | CDC activity improved | |
| Lys326Ala/Glu333Ala | Increased C1q binding | CDC |
| Lys326Met/Glu333Ser | Increased CDC activity | CDC |
| Cys221Asp/Asp222Cys | Increased C1q binding | CDC |
| Ser267Glu, His268Phe, | Increased C1q binding | CDC |
| and Ser324Thr | | |
| Ser267Glu/His268Glu/ | Enhanced C1q binding | CDC |
| Ser324Thr | affinity | |
| His268Phe and Ser324Thr | Improved CDC | CDC |
| Glu345Arg | Increased C1q binding | CDC |
| Glu345Arg/Glu430Gly/ | Improved CDC | CDC |
| Ser440Tyr | | |

In some embodiments, the antibodies or conjugates herein comprising a variant Fc region exhibit improved binding to at least one of Fcγ receptors CD 16a, CD32a, CD32b, CD32c, and CD64 as compared to a polypeptide construct comprising a native Fc region. In some cases, the antibodies or conjugates described herein exhibit improved binding to CD16a, CD32a, CD32b, CD32c, and CD64 Fcγ receptors. In some embodiments, the Fc variant exhibits improved binding to an Fc receptor of the subject compared to the wild-type human IgG Fc region. In some instances, an Fc variant has improved binding to an Fcγ receptor by a factor of 10%, 20% 30%, 40%, 50%, 60%, 70%, 80%, 90%, 100% or more as compared to the wildtype. In some embodiments, the improved binding is for any one or more Fcγ receptor, e.g., CD16a, CD32a, CD32b, CD32c, or CD64. In some embodiments, the improved in binding to an Fc7 receptor results in an improvement of ADCC. In some embodiments, antibodies or conjugates comprising an Fc variant as described herein exhibit at least a 5%, 10%, 15%, 20%, 30%, 40%, 50%, 60%, 70%, 80%, 90% or greater improvement in ADCC.

In some embodiments, the Fc variant exhibits an improvement of phagocytosis (ADCP) compared to the wild-type human IgG Fc region. Such Fc variants exhibit an improvement in phagocytosis compared to its wild-type human IgG Fc region, wherein the improvement of phagocytosis activity is e.g., by a factor of 10%, 20%, 30%, 40%, 50%, 60%, 70%, 80%, 90%, 100% or more.

Complement-directed cytotoxicity, which is also referred to herein as CDC, refers to a form of cytotoxicity in which the complement cascade is activated by the complement component C1q binding to antibody Fc. In some embodiments, the antibodies and conjugates comprising a non-native Fc region described herein exhibit at least a 5%, 10%, 15%, 20%, 30%, 40%, 50%, 60%, 70%, 80%, 90%, 100% or greater improvement in C1q binding compared to the antibody or conjugate comprising a wild-type Fc region. In some cases, antibodies or conjugates comprising a non-native Fc region as described herein exhibit improved CDC as compared to antibodies or conjugates comprising a wild-type Fc region. In some embodiments, antibodies or conjugates comprising a non-native Fc region as described herein exhibit at least a 5%, 10%, 15%, 20%, 30%, 40%, 50%, 60%, 70%, 80%, 90% 100% or greater improvement in CDC compared to antibodies or conjugates comprising a wild-type Fc region.

In some cases, antibodies or conjugates comprising a non-natural Fc variant as described herein exhibit improved CDC and also are characterized by improved binding (=to one or more of human FcγRI (CD64), FcγRIIa (CD32a), FcγRIIb (CD32b), FcγRIIc (CD32c), FcγRIIIa (CD16a), and FcγRIIIb (CD16b). In some embodiments, the Fc region of the antibodies or conjugates described herein has wild-type or native CDC activity. In some embodiments, the Fc region of the antibodies or conjugates described herein has wild-type or native CDC activity and is characterized by improved binding to one or more of human FcγRI (CD64), FcγRIIa (CD32a), FcγRIIb (CD32b), FcγRIIc (CD32c), FcγRIIIa (CD16a), and FcγRIIIb (CD16b).

In any of the embodiments herein, the nectin-4 antibody and nectin-4 antibody conjugates can be engineered to improve antibody-dependent cell-mediated cytotoxicity (ADCC), antibody-dependent cell-mediated phagocytosis (ADCP) and complement dependent cytolysis (CDC) activity. In any of the embodiments herein, the antibody and conjugates may comprise two heavy chains and wherein at least one of the two or both heavy chains of the antibody is non-fucosylated. In further embodiments, the antibody and conjugates may be produced in a cell line having an alpha1,6-fucosyltransferase (Fut8) knockout. In some further embodiments, the antibody and conjugates may be produced in a cell line overexpressing γ1,4-N-acetylglycosminyltransferase III (GnT-III). In further embodiments, the cell line additionally overexpresses Golgi μ-mannosidase II (ManII). In any of the embodiments herein, the antibody and conjugates may comprise at least one amino acid substitution in the Fc region that improves ADCC activity.

In any of the embodiments herein, the nectin-4 antibody and nectin-4 antibody conjugates may comprise at least one amino acid substitution in the Fc region that improves ADCC activity. Some exemplary Fc modifications include the following combinations of amino acid substutions in the Fc region of human IgGs, e.g., L235V/F243L/R292P/Y300L/P396L, S239D/I332E, S239D/A330L/I332E, P247I/A339Q, S298A/E333A/K334A (Liu et al., Fc—engineering for modulated effector functions—improving antibodies for cancer treatment, Antibodies 2020, 9, 64; Kellner et al., Modulating cytotoxic effector functions by Fc engineering to improve cancer therapy, Transfus. Med. Hemother. 2017; 44: 327-336).

In some embodiments, a Q-tag of the present disclosure is attached to the nectin-4 antibody. In some embodiments, a Q-tag of the present disclosure is attached to the nectin-4 antibody and the Q tag serves as the component to conjugate an oligonucleotide, such as a CpG oligonucleotide, which may be conjugated with a linker to the Q tag.

In some embodiments, the Q-tag is attached to the heavy chain of the nectin-4 antibody. In some embodiments, the Q-tag is attached to the heavy chain of the nectin-4 antibody via a linker (e.g., an amino acid or other chemical linker). In some embodiments, the Q-tag is attached to the heavy chain of the nectin-4 antibody (e.g., fused in frame with the heavy chain). In some embodiments, the Q-tag is attached at the C-terminus of the heavy chain of the nectin-4 antibody. In some embodiments, the Q-tag is fused to the C-terminus of the heavy chain of the nectin-4 antibody (e.g., in frame and contiguous with the amino acid sequence of the C-terminus). In some embodiments, the Q-tag is within the Fc domain of the nectin-4 antibody. In some embodiments, the Q tag is naturally occurring. For example, mutation of N297 to N297A exposes Q295 of the antibody, where the conjugation could occur. In certain embodiments wherein the Fc region comprises an N297A substitution, the conjugate further comprises an immunomodulating oligonucleotide P attached to the Q295 residue as shown in the following formula

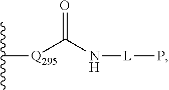

wherein L is a linker moiety connected to Q295 via an amide bond.

In some embodiments, the Q-tag comprises one or more sequences shown in Table 16.

TABLE 16
Q-tag Peptide Sequences

| SEQ ID NO. | Peptide Sequences |
|---|---|
| 39 | LSLSPGLLQGG-OH |
| 40 | WPAQGPT |
| 41 | WPQGPT |
| 42 | WAPQGPT |
| 43 | WAQGPT |
| 44 | TPGQAPW |
| 45 | PNPQLPF |
| 46 | RPQQF |
| 47 | RPQGF |
| 48 | RPQGFPP |
| 49 | RPQGFGPP |
| 50 | RPRPQQF |
| 51 | LSQSKVLG |
| 52 | WGGQLL |
| 53 | WALQRPHYSYPD |
| 54 | WALQRPYTLTES |
| 55 | WALQGPYTLTES |

In some embodiments, an antibody or conjugate of the present disclosure can be delivered as a naked protein-drug conjugate, or as a protein-drug conjugate formulated with a carrier and delivered, e.g., as encapsulated or as part of a nanocarrier, nanoparticle, liposome, polymer vesicle, or viral envelope. In some embodiments, an antibody or conjugate of the present disclosure can be delivered intracellularly, e.g., by conjugation to a protein-transduction domain or mimic. In some embodiments, an antibody or conjugate of the present disclosure can be delivered by electroporation or microinjection.

In some embodiments, the nectin-4 antibody or conjugate has one or more effector functions, including without limitation ADCC and/or ADCP. In some embodiments, the nectin-4 antibody or conjugate comprises a human Fc region, e.g., a human IgG Fc region. In some embodiments, the nectin-4 antibody or conjugate comprises a wild-type human IgG1, IgG2, or IgG4 Fc region. In some embodiments, the nectin-4 antibody or conjugate comprises the antibody constant domain sequence of SEQ ID NO:178.

In certain embodiments, the nectin-4 antibodies or nectin-4 antibody conjugates described herein comprise an IgG Fc region selected from the group consisting of an IgG1 Fc region, an IgG2 Fc region, and an IgG4 Fc region. In some embodiments, the Fc region is a wild-type human IgG1, IgG2, or IgG4 Fc region. In some embodiments, the IgG Fc region is a human IgG Fc region comprising one or more amino acid substitutions that increaseor enhance one or more effector functions, as compared with the effector function(s) of a human Fc region that lacks the amino acid substitution(s). For example, in some embodiments, an IgG Fc domain is mutated to increase effector functions, to create an “active” or enhanced Fc domain. In some embodiments, to change or enhance an antibody-dependent effector function, such as ADCC, CDC, ADCP, or any combinations thereof, the Fc domain are of the IgG class and comprise one or more amino acid substitutions as described and listed in Table 27.

In some embodiments, the nectin-4 antibody or conjugate of the present disclosure comprises an antibody constant domain. In some embodiments, the nectin-4 antibody or conjugate of the present disclosure comprises an antibody heavy chain constant domain and/or antibody light chain constant domain listed in Table 17. In some embodiments, the nectin-4 antibody or conjugate of the present disclosure comprises an antibody heavy chain constant domain selected from the group consisting of SEQ ID NOs:92-107, 111, 112, 178, and 494-497. In some embodiments, the nectin-4 antibody of the present disclosure comprises an antibody heavy chain constant domain with a Q-tag at the C-terminus of the Fc region, e.g., as shown in SEQ ID No: 95 or 178. In some embodiments, the nectin-4 antibody or conjugate of the present disclosure comprises two antibody heavy chains, each with a constant domain, wherein each of the two antibody heavy chains comprises a Q-tag at the C-terminus of the Fc region, e.g., as shown in SEQ ID No: 95 or 178. In some embodiments, the nectin-4 antibody or conjugate of the present disclosure comprises two antibody heavy chains, each with a constant domain, wherein only one of the two antibody heavy chains comprises a Q-tag at the C-terminus of the Fc region, e.g., as shown in SEQ ID No: 95 or 178. In some embodiments, the nectin-4 antibody or conjugate of the present disclosure comprises an antibody light chain constant domain selected from the group consisting of SEQ ID Nos: 108-110. In some embodiments, the nectin-4 antibody or conjugate of the present disclosure comprises a non-fucosylated or reduced fucosylation of the Fc domain. In some embodiments, the nectin-4 antibody or conjugate of the present disclosure comprises a non-fucosylated or reduced fucosylation of the Fc domain and the antibody constant comain comprises one of SEQ ID NOs:95, 112, and 178.

TABLE 17
Antibody constant domain sequences

| | SEQ ID | |
|---|---|---|
| Name | NO: | Sequence |
| IgG1 wildtype | 92 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRW |
| | | QQGNVFSCSVMHEALHNHYTQKSLSLSPG |
| IgG1_AAA_N297A | 93 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPEAAGAPSV |
| | | FLFPPKPKDTLMISRTPEVTCVVVDVSHEDPEVKFNWYVD |
| | | GVEVHNAKTKPREEQYASTYRVVSVLTVLHQDWLNGKEY |
| | | KCKVSNKALPAPIEKTISKAKGQPREPQVYTLPPSREEMTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPG |
| IgG1_AAA | 94 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPEAAGAPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSREEMTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPG |
| IgG1_AAA + Q-tag | 95 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPEAAGAPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSREEMTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPGRPQGFGPP |
| IgG1_N297A | 96 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGPSV |
| | | FLFPPKPKDTLMISRTPEVTCVVVDVSHEDPEVKFNWYVD |
| | | GVEVHNAKTKPREEQYASTYRVVSVLTVLHQDWLNGKEY |
| | | KCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRW |
| | | QQGNVFSCSVMHEALHNHYTQKSLSLSPG |
| IgG1_D265A | 97 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGPSV |
| | | FLFPPKPKDTLMISRTPEVTCVVVAVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRW |
| | | QQGNVFSCSVMHEALHNHYTQKSLSLSPG |
| IgG1_N297A/D265A | 98 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGPSV |
| | | FLFPPKPKDTLMISRTPEVTCVVVAVSHEDPEVKFNWYVD |
| | | GVEVHNAKTKPREEQYASTYRVVSVLTVLHQDWLNGKEY |
| | | KCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRW |
| | | QQGNVFSCSVMHEALHNHYTQKSLSLSPG |
| IgG2 | 99 | ASTKGPSVFPLAPCSRSTSESTAALGCLVKDYFPEPVTVSW |
| | | NSGALTSGVHTFPAVLQSSG |
| | | LYSLSSVVTVPSSNFGTQTYTCNVDHKPSNTKVDKTVERK |
| | | CCVECPPCPAPPVAGPSVFL |
| | | FPPKPKDTLMISRTPEVTCVVVDVSHEDPEVQFNWYVDGV |
| | | EVHNAKTKPREEQFNSTFRV |
| | | VSVLTVVHQDWLNGKEYKCKVSNKGLPAPIEKTISKTKGQ |
| | | PREPQVYTLPPSREEMTKNQ |
| | | VSLTCLVKGFYPSDIAVEWESNGQPENNYKTTPPMLDSDG |
| | | SFFLYSKLTVDKSRWQQGNV |
| | | FSCSVMHEALHNHYTQKSLSLSPG |
| IgG2Da | 100 | ASTKGPSVFPLAPCSRSTSESTAALGCLVKDYFPEPVTVSW |
| | | NSGALTSGVHTFPAVLQSSGLYSLSSVVTVPSSNFGTQTYT |
| | | CNVDHKPSNTKVDKTVERKCCVECPPCPAPPVAGPSVFLFP |
| | | PKPKDTLMISRTPEVTCVVVDVSHEDPEVQFNWYVDGVEV |
| | | HNAKTKPREEQFNSTFRVVSVLTVVHQDWLNGKEYKCKV |
| | | SNKGLPSSIEKTISKTKGQPREPQVYTLPPSREEMTKNQVSL |
| | | TCLVKGFYPSDIAVEWESNGQPENNYKTTPPMLDSDGSFFL |
| | | YSKLTVDKSRWQQGNVFSCSVMHEALHNHYTQKSLSLSP |
| | | G |
| IgG2Da_N297A | 101 | ASTKGPSVFPLAPCSRSTSESTAALGCLVKDYFPEPVTVSW |
| | | NSGALTSGVHTFPAVLQSSGLYSLSSVVTVPSSNFGTQTYT |
| | | CNVDHKPSNTKVDKTVERKCCVECPPCPAPPVAGPSVFLFP |
| | | PKPKDTLMISRTPEVTCVVVDVSHEDPEVQFNWYVDGVEV |
| | | HNAKTKPREEQFASTFRVVSVLTVVHQDWLNGKEYKCKV |
| | | SNKGLPSSIEKTISKTKGQPREPQVYTLPPSREEMTKNQVSL |
| | | TCLVKGFYPSDIAVEWESNGQPENNYKTTPPMLDSDGSFFL |
| | | YSKLTVDKSRWQQGNVFSCSVMHEALHNHYTQKSLSLSP |
| | | G |
| IgG2_N297A | 102 | ASTKGPSVFPLAPCSRSTSESTAALGCLVKDYFPEPVTVSW |
| | | NSGALTSGVHTFPAVLQSSG |
| | | LYSLSSVVTVPSSNFGTQTYTCNVDHKPSNTKVDKTVERK |
| | | CCVECPPCPAPPVAGPSVFL |
| | | FPPKPKDTLMISRTPEVTCVVVDVSHEDPEVQFNWYVDGV |
| | | EVHNAKTKPREEQFASTFRV |
| | | VSVLTVVHQDWLNGKEYKCKVSNKGLPAPIEKTISKTKGQ |
| | | PREPQVYTLPPSREEMTKNQ |
| | | VSLTCLVKGFYPSDIAVEWESNGQPENNYKTTPPMLDSDG |
| | | SFFLYSKLTVDKSRWQQGNV |
| | | FSCSVMHEALHNHYTQKSLSLSPG |
| IgG2Da_D265A | 103 | ASTKGPSVFPLAPCSRSTSESTAALGCLVKDYFPEPVTVSW |
| | | NSGALTSGVHTFPAVLQSSGLYSLSSVVTVPSSNFGTQTYT |
| | | CNVDHKPSNTKVDKTVERKCCVECPPCPAPPVAGPSVFLFP |
| | | PKPKDTLMISRTPEVTCVVVAVSHEDPEVQFNWYVDGVEV |
| | | HNAKTKPREEQFNSTFRVVSVLTVVHQDWLNGKEYKCKV |
| | | SNKGLPSSIEKTISKTKGQPREPQVYTLPPSREEMTKNQVSL |
| | | TCLVKGFYPSDIAVEWESNGQPENNYKTTPPMLDSDGSFFL |
| | | YSKLTVDKSRWQQGNVFSCSVMHEALHNHYTQKSLSLSP |
| | | G |
| IgG4_S228P | 104 | ASTKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEFLGGPSVFLF |
| | | PPKPKDTLMISRTPEVTCVVVDVSQEDPEVQFNWYVDGVE |
| | | VHNAKTKPREEQFNSTYRVVSVLTVLHQDWLNGKEYKCK |
| | | VSNKGLPSSIEKTISKAKGQPREPQVYTLPPSQEEMTKNQV |
| | | SLTCLVKGFYPSDIAVEWESNGQPENNYKTTPPVLDSDGSF |
| | | FLYSRLTVDKSRWQEGNVFSCSVMHEALHNHYTQKSLSLS |
| | | LG |
| IgG4_S228P_D265A | 105 | ASTKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEFLGGPSVFLF |
| | | PPKPKDTLMISRTPEVTCVVVAVSQEDPEVQFNWYVDGVE |
| | | VHNAKTKPREEQFNSTYRVVSVLTVLHQDWLNGKEYKCK |
| | | VSNKGLPSSIEKTISKAKGQPREPQVYTLPPSQEEMTKNQV |
| | | SLTCLVKGFYPSDIAVEWESNGQPENNYKTTPPVLDSDGSF |
| | | FLYSRLTVDKSRWQEGNVFSCSVMHEALHNHYTQKSLSLS |
| | | LG |
| IgG4_S228P, L235E | 106 | ASTKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEFEGGPSVFLF |
| | | PPKPKDTLMISRTPEVTCVVVDVSQEDPEVQFNWYVDGVE |
| | | VHNAKTKPREEQFNSTYRVVSVLTVLHQDWLNGKEYKCK |
| | | VSNKGLPSSIEKTISKAKGQPREPQVYTLPPSQEEMTKNQV |
| | | SLTCLVKGFYPSDIAVEWESNGQPENNYKTTPPVLDSDGSF |
| | | FLYSRLTVDKSRWQEGNVFSCSVMHEALHNHYTQKSLSLS |
| | | LG |
| IgG4_S228P,N297A | 107 | ASTKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEFLGGPSVFLF |
| | | PPKPKDTLMISRTPEVTCVVVDVSQEDPEVQFNWYVDGVE |
| | | VHNAKTKPREEQFASTYRVVSVLTVLHQDWLNGKEYKCK |
| | | VSNKGLPSSIEKTISKAKGQPREPQVYTLPPSQEEMTKNQV |
| | | SLTCLVKGFYPSDIAVEWESNGQPENNYKTTPPVLDSDGSF |
| | | FLYSRLTVDKSRWQEGNVFSCSVMHEALHNHYTQKSLSLS |
| | | LG |
| IgG1_wt + Q-tag | 178 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRW |
| | | QQGNVFSCSVMHEALHNHYTQKSLSLSPGRPQGFGP |
| | | P |
| IgG1 | 494 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELVGGPSV |
| | | FLLPPKPKDTLMISRTPEVTCVVVDVSHEDPEVKFNWYVD |
| | | GVEVHNAKTKPPEEQYNSTLRVVSVLTVLHQDWLNGKEY |
| | | KCKVSNKALPAPIEKTISKAKGQPREPQVYTLPPSREEMTK |
| | | NQVSLTCLVKGFYPSDIAVEWESNGQPENNYKTTPLVLDS |
| | | DGSFFLYSKLTVDKSRWQQGNVFSCSVMHEALHNHYTQK |
| | | SLSLSPG |
| IgG1 G236A | 111 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLAGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSREEMTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPG |
| IgG1 G236A + Q-tag | 112 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLAGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSREEMTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPGRPQGFGPP |
| IgG1_S239E/I332E | 495 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGPDV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPEEKTISKAKGQPREPQVYTLPPSREEMTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPG |
| IgG1_S239D/A330L/I332E | 496 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGPDV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPLPEEKTISKAKGQPREPQVYTLPPSREEMTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPG |
| IgG1_P247I/A339Q | 497 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGPSV |
| | | FLFPPKIKDTLMISRTPEVTCVVVDVSHEDPEVKFNWYVDG |
| | | VEVHNAKTKPREEQYN |
| | | STYRVVSVLTVLHQDWLNGKEYKCKVSNKALPAPIEKTIS |
| | | KQKGQPREPQVYTLPPSREEMTKNQVSLTCLVKGFYPSDIA |
| | | VEWESNGQPENNYKTTPPVLDSDGSFFLYSKLTVDKSRWQ |
| | | QGNVFSCSVMHEALHNHYTQKSLSLSPG |
| Human Kappa | 108 | RTVAAPSVFIFPPSDEQLKSGTASVVCLLNNFYPREAKVQW |
| | | KVDNALQSGNSQESVTEQDSKDSTYSLSSTLTLSKADYEK |
| | | HKVYACEVTHQGLSSPVTKSFNRGEC |
| Human Lambda IGLC1 | 109 | GQPKANPTVTLFPPSSEELQANKATLVCLISDFYPGAVTVA |
| | | WKADGSPVKAGVETTKPSKQSNNKYAASSYLSLTPEQWK |
| | | SHRSYSCQVTHEGSTVEKTVAPTECS |
| Human Lambda IGLC2 | 110 | GQPKAAPSVTLFPPSSEELQANKATLVCLISDFYPGAVTVA |
| | | WKADSSPVKAGVETTTPSKQSNNKYAASSYLSLTPEQWKS |
| | | HRSYSCQVTHEGSTVEKTVAPTECS |

In another aspect of the present disclosure, provided herein is a conjugate comprising a nectin-4 antibody or antigen-binding fragment thereof and one or more immunomodulating oligonucleotides (P), wherein the nectin-4 antibody or antigen-binding fragment is linked to one or more Q-tag peptides (Q) comprising the amino acid sequence RPQGF (SEQ ID NO:47), wherein each immunomodulating oligonucleotide is linked to a Q-tag peptide via an amide bond with the glutamine residue of the Q-tag peptide and a linker (L) as shown in formula (A),

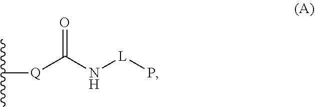

wherein:
each Q independently comprises a Q-tag peptide sequence RPQGF (SEQ ID NO:47);
each L is independently a bond or a linker moiety

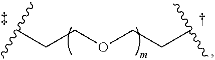

wherein m is an integer ranging from about 0 to about 50 (such as about 0 to about 10, about 0 to about 30, about 10 to about 30, about 20 to about 30, and values in ranges therebetween), and wherein [symbol] † indicates the point of attachment to P, and [symbol] ‡ indicates the point of attachment to the rest of the conjugate connected to Q via an amide bond with the glutamine residue;
and each P is independently an immunomodulating oligonucleotide comprising the structure

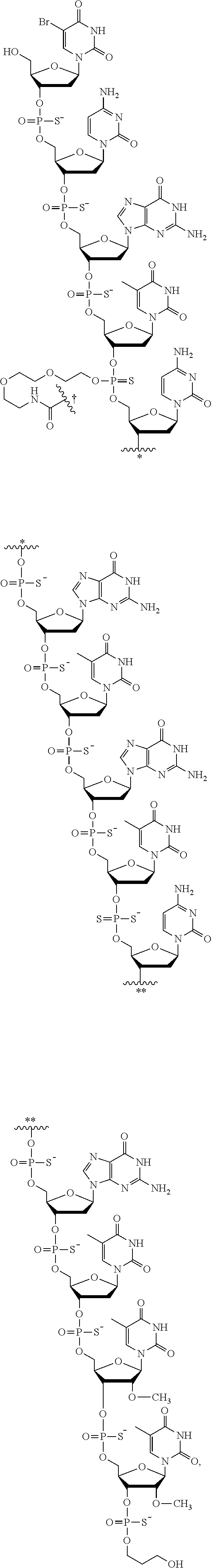

wherein [symbol] * and [symbol] ** indicate the points of attachment within the oligonucleotide, and wherein [symbol] † indicates the point of attachment to L;
wherein Ab comprises a heavy chain variable (VH) domain and a light chain variable (VL) domain, wherein the VH domain comprises CDR-H1, CDR-H2, and CDR-H3 sequences from a VH domain sequence selected from the group of sequences disclosed in Table 7 according to Kabat, Chothia, or IMGT;
wherein the VL domain comprises CDR-L1, CDR-L2, and CDR-L3 sequences from a VL domain sequence selected from the group of sequences disclosed in Table 8 according to Kabat, Chothia, or IMGT.

In still other aspects, provided herein is a conjugate comprising a protein, at least one Q tag peptide sequence comprising a glutamine residue, and at least one immunomodulatory oligonucleotide, wherein the Q-tag peptide sequence is naturally occurring or synthetic, and wherein the immunomodulatory oligonucleotide is linked to the Q-tag via an amide bond with the glutamine residue, wherein at least one Q-tag peptide sequence is selected from the group consisting of SEQ ID NOs: 39-55.

In some embodiments, the immunomodulatory oligonucleotide has a sequence selected from the group consisting of the oligonucleotides of Table 4 and Table 6.

In some embodiments, the nectin-4 antibody comprises at least one Q tag peptide sequence comprising a glutamine residue. In some embodiments, the Q tag peptide sequence is naturally occurring or synthetic. In certain embodiments, the Q tag peptide sequence is an internal reactive glutamine exposed by an amino acid substitution. In further embodiments, the Q tag is fused to the C-terminus of the heavy chain of the protein. In still further embodiments, at least one of the at least one Q tag peptide sequences is elected from the group consisting of SEQ ID NOs: 39-55.

In another aspect of the present disclosure, provided herein are nectin-4 antibodies of formula (B)

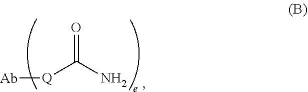

wherein:
each Q is independently a Q-tag comprising a peptide sequence with at least one glutamine residue;
Ab is the nectin-4 antibody or antigen-binding fragment thereof; and
e is an integer from 1 to 20.

The nectin-4 antibodies of formula (B) may be precursors to the antibody-oligonucleotide conjugates of formula (A) as described above. Accordingly, the properties and embodiments of the antibodies as described in the previous aspect of formula (A) may be the same or different from the properties and/or embodiments of the antibodies of formula (B).

In some embodiments of the present aspect, the nectin-4 antibody or fragment thereof is a monoclonal antibody or fragment thereof. In certain embodiments, the nectin-4 antibody or fragment thereof is a Fab, F(ab′)₂, Fab′-SH, Fv, scFv, single domain, single heavy chain, or single light chain antibody or antibody fragment. In other embodiments, the nectin-4 antibody or fragment thereof is a humanized, human, or chimeric antibody or fragment thereof.

In some embodiments, the nectin-4 antibody and nectin-4 antibody conjugates comprise an Fc region. In certain embodiments wherein the nectin-4 antibody or nectin-4 antibody conjugate comprises an Fc region, the Fc region is a human Fc region selected from the group consisting of an IgG1 Fc region, an IgG2 Fc region, and an IgG4 Fc region.

In certain embodiments of the present aspect, the Fc region is:
(a) a human IgG1 Fc region comprising L234A, L235A, and/or G237A substitutions, amino acid position numbering according to EU index;
(b) a human IgG2 Fc region comprising A330S and/or P331S substitutions, amino acid position numbering according to EU index; or
(c) a human IgG4 Fc region comprising S228P and/or L235E substitutions, amino acid position numbering according to EU index.

In some embodiments, the Fc region further comprises an N297A substitution, amino acid position numbering according to EU index. In other embodiments, the Fc region further comprises a D265A substitution, amino acid position numbering according to EU index. In yet further embodiments, the nectin-4 antibody comprises a human lambda light chain. In other embodiments, the nectin-4 antibody comprises a human kappa light chain.

In some embodiments, at least one Q-tag is attached to the heavy chain of the nectin-4 antibody. In certain embodiments, at least one Q-tag is fused to the C-terminus of the heavy chain of the nectin-4 antibody. In other embodiments, at least one Q-tag is attached to the light chain of the nectin-4 antibody. In still further embodiments, at least one Q-tag is within the Fc domain.

In some embodiments of the present aspect, the nectin-4 antibody is linked to from 1 to 20 Q-tags (Q). In certain embodiments, the number of Q-tags linked to the nectin-4 antibody conjugate is an integer of about 1, about 2, about 3, about 4, about 5, about 6, about 7 about 8, about 9, about 10, about 11 about 12, about 13, about 14, about 15, about 16, about 17, about 18, about 19, or about 20. In certain other embodiments, 1 or 2 Q-tags is/are linked to the nectin-4 antibody or antigen-binding fragment. In yet other embodiments, the number of Q-tags linked to the nectin-4 antibody conjugate is an integer from 1 to 10, from 10 to 20, from 5 to 10, from 10 to 15, from 15 to 20, or from 1 to 5.

In still further embodiments of the present aspect, which may be combined with any of the preceding embodiments, each Q tag independently comprises or is a peptide sequence selected from the group consisting of SEQ ID NOs: 39-55. In some embodiments, each Q tag independently comprises or is a peptide sequence selected from the group consisting of the peptide sequences of Table 16. In other embodiments of the present aspect, each Q tag independently comprises or is a peptide sequence selected from the group consisting of SEQ ID NOs: 40-55. In yet other embodiments, each Q tag independently comprises or is a peptide sequence selected from the group consisting of SEQ ID NOs: 47-49. In some embodiments, the Q-tag comprises LLQGG (SEQ ID NO:172), GGGLLQGG (SEQ ID NO:173), RPQGF (SEQ ID NO:47), or RPQGFGPP (SEQ ID NO:49). In some embodiments of the present aspect, each Q is independently a Q-tag comprising a peptide sequence RPQGF (SEQ ID NO:47). In certain embodiments, each Q-tag comprising a peptide sequence RPQGF (SEQ ID NO:47) is selected from the group consisting of RPQGF (SEQ ID NO:47), RPQGFPP (SEQ ID NO:48), and RPQGFGPP (SEQ ID NO:49). In certain embodiments, each Q tag independently comprises or is a peptide sequence RPQGFGPP (SEQ ID NO:49).

In some embodiments, the nectin-4 antibody of the present disclosure is conjugated to an agent. In some embodiments, the agent is a cytotoxic agent. Exemplary cytotoxic agents are described in U.S. Pat. No. 8,088,378 B2, the contents of which are incorporated herein by reference. In some embodiments, the agent is a label.

In some embodiments, the agent is a moiety that modulates the immune system. For example, the moiety may target and/or modulate the function of a cell expressing nectin-4 on its surface, such as a small molecule that modulates a cellular signaling pathway of the cell expressing nectin-4, e.g., an IDO/TDO inhibitor, AhR inhibitor, arginase inhibitor, A2aR inhibitor, TLR agonists, STING agonist, or Rig-1 agonist. In some embodiments, the moiety may recruit another macromolecule or cell into proximity with a cell expressing nectin-4 on its surface. In some embodiments, the moiety comprises a cytokine, e.g., IL2, IL7, IL-10, IL15, or IFN. In some embodiments, the moiety (e.g., a small molecule) modulates the activity of a cytokine, e.g., IL2, IL7, IL-10, IL15, or IFN. In some embodiments, the moiety comprises a cancer vaccine (comprising, e.g., DNA, RNA, peptide, or other cellular component(s)).

In some embodiments, the nectin-4 antibody is a bispecific antibody. In some embodiments, the nectin-4 antibody is a multispecific antibody. In some embodiments, the nectin-4 antibody is a bispecific or multispecific antibody comprising a first VL domain and a first VH domain which specifically binds human nectin-4 and at least a second VL domain and a second VH domain which specifically binds another target than human nectin-4. In some embodiments, the nectin-4 antibody is a bispecific or multispecific antibody that is conjugated to a therapeutic agent, such as an oligonucleotide, a CpG oligonucleotide, or other agent.

Where the antibody comprises an Fc region, the carbohydrate attached thereto may be altered. For example, antibodies with a mature carbohydrate structure that lacks fucose attached to an Fc region of the antibody are described in US 2003/0157108. See, also, US 2004/0093621. Antibodies with a bisecting N-acetylglucosamine (GlcNAc) in the carbohydrate attached to an Fc region of the antibody are referenced in WO 2003/011878 and U.S. Pat. No. 6,602,684. Antibodies with at least one galactose residue in the oligosaccharide attached to an Fc region of the antibody are reported in WO 1997/30087. See, also, WO 1998/58964 and WO 1999/22764 concerning antibodies with altered carbohydrate attached to the Fc region thereof. See also US 2005/0123546 on antigen-binding molecules with modified glycosylation.

In certain embodiments, a glycosylation variant comprises an Fc region, wherein a carbohydrate structure attached to the Fc region lacks fucose or has reduced fucose. Such variants are associated with improved ADCC function. Optionally, the Fc region further comprises one or more amino acid substitutions therein which further improve ADCC, for example, substitutions at positions 298, 333, and/or 334 of the Fc region (EU numbering of residues). Examples of publications related to “defucosylated,” “non-fucosylated,” “afucosylated,” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; Okazaki et al. J. Mol. Biol. 336:1239-1249 (2004); Yamane-Ohnuki et al. Biotech. Bioeng. 87: 614 (2004). Examples of cell lines producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (Yamane-Ohnuki et al. Biotech. Bioeng. 87: 614 (2004)), and cells overexpressing γ1,4-N-acetylglucosaminyltransferase III (GnT-III) and Golgi μ-mannosidase II (ManII). Other methods to produce defucosylated antibodies can include expression in the presence of 2-fluorofucose or 5-alkynylfucose derivatives in the cell line expression systems (transient or stable) (Okeley et al. Appl. Biol. Sciences. 110 (14) 5404-5409 (2013).

In some embodiments, the nectin-4 antibodies of the disclosure, or the nectin-4 antibody conjugates, have reduced fucose relative to the amount of fucose on the same antibody or antibody conjugate produced in a cell line that provides fucosylation of an antibody, such as a mammalian cell line, for example a wild-type CHO cell line or a HEK293 cell line. For example, the antibody has a lower amount of fucose than it would otherwise have if produced by native CHO cells (e.g., a CHO cell that produce a native glycosylation pattern, such as, a CHO cell containing a native FUT8 gene). In certain embodiments, a nectin-4 antibody provided herein is one wherein less than any of about 50%, about 40%, about 30%, about 20%, about 10%, about 5% or about 1% of the N-linked glycans thereon comprise fucose. In certain embodiments, a nectin-4 antibody or antibody conjugate provided herein is one wherein none of the N-linked glycans thereon comprise fucose, i.e., wherein the antibody is completely without fucose, or has no fucose or is non-fucosylated or is afucosylated. The amount of fucose can be determined by calculating the average amount of fucose within the sugar chain at Asn297, relative to the sum of all glycostructures attached to Asn297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. In some embodiments, at least one or two of the heavy chains of the antibody is non-fucosylated.

### IV General Oligonucleotide Synthesis

### General Scheme

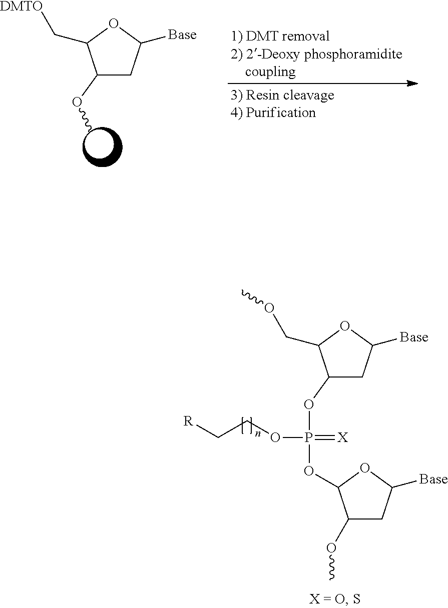

### Experimental Detail

Automated oligonucleotide synthesis (1 μmol scale) was carried out on MerMade 6 or 12 with the following reagents and solvents:
Oxidizer—0.02M 12 in THF/pyridine/H₂O (60 s oxidation per cycle),
Sulfurizing Reagent II—dithiazole derivative/pyridine/acetonitrile (0.05 M, in 6:4 pyridine:acetonitrile) (60 s per cycle)
Deblock—3% trichloroacetic acid (2× 40 s deblocks per cycle),
Cap Mix A—THF/2,6-lutidine/Ac20 (60 s capping per cycle), and
Cap Mix B—16% methyl imidazole in THF (60 s capping per cycle)

Exceptions to standard oligonucleotide synthesis conditions were as follows:
CPG supports with a non-nucleosidic linker called Uny-linker was used.
All 2′-deoxyribose-phosphoramidites were resuspended to 100 mM in 100% anhydrous acetonitrile prior to synthesis, except some of the modified 2′-deoxy-phosphoramidites were dissolved to 100 mM in THF/acetonitrile mixture (1:4) depend on the solubility of the starting material.
Phosphoramidite activation was performed with a 2.5-fold molar excess of 5-benzylthio-1H-tetrazole (BTT). Activated 2′-deoxyribose-phosphoramidites were coupled for 2× 1 minute coupling per insertion and modified phosphoramidites were coupled for 2× 3 minute coupling per insertion.
Sulfurization of the backbone was performed with 0.05M Sulfurizing Reagent II in pyridine/acetonitrile (6:4) for 1 min.

### Oligonucleotide Deprotection & Purification Protocol:

Following automated oligonucleotide synthesis, solid support and base protecting groups (such as A-Bz, C—Ac, G-iBu, etc.) and methyl esters of phosphotriesters were cleaved and de-protected in 1 mL of AMA (1:1 ratio of 36% aq. ammonia and 40% methylamine in methanol) for 2 h or more at room temperature followed by centrifugal evaporation.

Crude oligonucleotide pellets were resuspended in 100 μL of 50% acetonitrile, briefly heated to 65° C. and vortexed thoroughly.

For oligonucleotide purification, 100 μL crude oligonucleotides were injected onto RP-HPLC with the following buffers/gradient:
Buffer A=50 mM TEAA in Water;
Buffer B=90% Acetontrile; and
Flow Rate=1 mL/min;
Gradient:
   0-2 min (100% Buffer A/0% Buffer B),
   2-42 min (0% to 60% Buffer B), and
   42-55 min (60% to 100% Buffer B).

### DBCO Conjugation and Purification Protocol:

DBCO NHS ester was conjugated to the crude 2′-deoxy DMT-oligonucleotide as described here. The crude oligonucleotide pellet was suspended into 45 μL DMSO, briefly heated to 65° C. and vortexed thoroughly. 5 μL of DIPEA was added followed by DBCO-NHS ester (30 eq), which was pre-dissolved in DMSO (1M). The reaction was allowed to stand for 10 minutes or until product formation was confirmed by MALDI. Total 80 μL of crude oligonucleotide samples were injected onto RP-HPLC with the following buffers/gradient:
Buffer A=50 mM TEAA in Water
Buffer B=90% Acetonitrile
Flow Rate=1 mL/min
Gradient:
   0-2 min (90% Buffer A/10% Buffer B)
   2-42 min (0% to 60% Buffer B)
   42-55 min (60% to 100% Buffer B).

Across the dominant RP-HPLC peaks, 0.5 mL fractions were collected and analyzed by MALDI-TOF mass spectrometry to confirm presence of desired mass. Mass-selected, purified fractions were frozen and lyophilized. Once dry, fractions were re-suspended, combined with corresponding fractions, frozen and lyophilized.

DMT Cleavage: lyophilized pellets were suspended in 20 μL of 50% acetonitrile and added 80 μL of acetic acid, samples were kept standing at room temperature forl h, frozen and lyophilized. The dried samples were re-dissolved in 20% acetonitrile and desalted through NAP 10 (Sephadex™-G25 DNA Grade) columns. Collected, pure fractions were frozen and lyophilized for final product.

### Methods for Attaching Oligonucleotides to Linking Moiety

### Cu-Catalyzed Click Reaction

### Copper-THPTA Complex Preparation

A 5 mM aqueous solution of copper sulfate pentahydrate (CuSO₄-5H₂O) and a 10 mM aqueous solution of tris(3-hydroxypropyltriazolylmethyl)amine (THPTA) were mixed 1:1 (v/v) (1:2 molar ratio) and allowed to stand at room temperature for 1 hour. This complex can be used to catalyze Huisgen cycloaddition, e.g., as shown in the general conjugation schemes below.

### General Procedure (100 nM Scale):

To a solution of 710 μL of water and 100 μL tert-butanol (10% of final volume) in a 1.7 mL Eppendorf tube was added 60 μL of the copper-THPTA complex followed by 50 μL of a 2 mM solution of the oligo, 60 μL of a 20 mM aqueous sodium ascorbate solution and 20 μL of a 10 mM solution of targeting moiety-azide. After thorough mixing the solution was allowed to stand at room temperature for 1 hour. Completion of the reaction was confirmed by gel analysis. The reaction mixture is added to a screw cap vial containing 5-10 fold molar excess of SiliaMetS® TAAcONa (resin bound EDTA sodium salt). The mixture is stirred for 1 hour. This mixture is then eluted through an illustra™Nap™-10 column Sephadex™. The resulting solution is then frozen and lyophilized overnight.

### Attachment Through Amide Linkage:

Conjugation through amidation may be performed under the amidation reaction conditions known in the art. See, e.g., Aaronson et al., Bioconjugate Chem. 22:1723-1728, 2011.

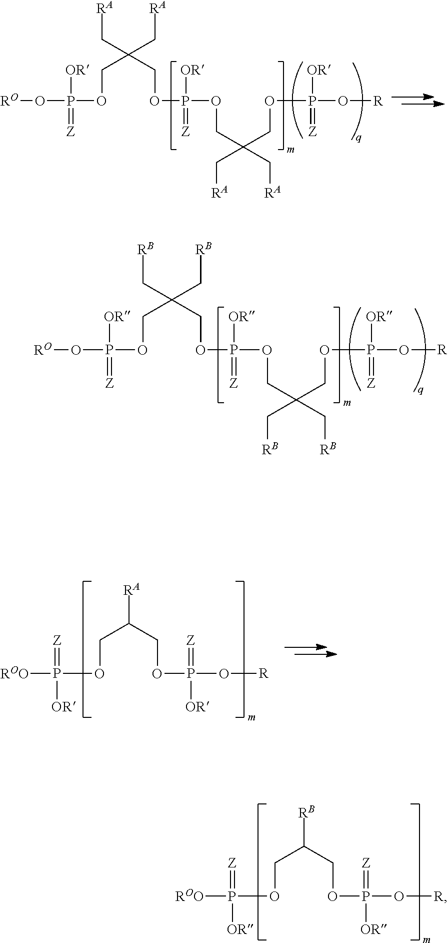

where
each q is 0 or 1;
each m is an integer from 0 to 5;
Z is O or S;
Rº is a bond to a nucleoside in an oligonucleotide;
R is a bond to H, a nucleoside in an oligonucleotide, to solid support, or to a capping group (e.g., —(CH₂)₃—OH);
each R′ is independently H, -Q¹-Q^{A1}, a bioreversible group, or a non-bioreversible group;
each R″ is independently H, -Q¹-Q^{A}-Q¹-T, a bioreversible group, or a non-bioreversible group;
each R^{A} is independently H or —OR^{C}, where R^{C} is -Q¹-Q^{A1}, a bioreversible group, or a non-bioreversible group;
each R^{B} is independently H or —OR^{D}, where R^{D} is QQ^{A}Q2-T, a bioreversible group, or a non-bioreversible group;
where
   each Q¹ is independently a divalent, trivalent, tetravalent, or pentavalent group, in which one valency is bonded to Q^{A} or Q^{A}, the second valency is open, and each of the remaining valencies, when present, is independently bonded to an auxiliary moiety;
   each Q² is independently a divalent, trivalent, tetravalent, or pentavalent group, in which one valency is bonded to Q^{A}, the second valency is bonded to T, and each of the remaining valencies, when present, is independently bonded to an auxiliary moiety;
   Q^{A} is optionally substituted C₂₋₁₂ heteroalkylene containing —C(O)—N(H)— or —N(H)—C(O)—;
   Q^{A1} is —NHR^{N1} or —COOR¹², where R^{N1} is H, N-protecting group, or optionally substituted C₁₋₆ alkyl, and R¹² is H, optionally substituted C₁₋₆ alkyl, or O⁻protecting group; and
   T is a linking moiety,
   provided that the starting materials contain at least one -Q¹-Q^{A1} and products contain -Q¹-QA-Q²-T.

Solution phase attachment:

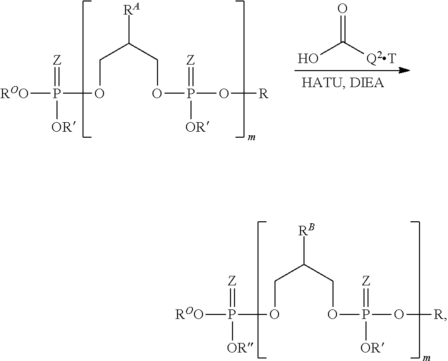

where
m is an integer from 0 to 5;
Z is O or S;
R^{O} is a bond to a nucleoside in an oligonucleotide;
R is a bond to H, a nucleoside in an oligonucleotide, or to a capping group;
each R′ is independently H, -Q¹-NH₂, a bioreversible group, or a non-bioreversible group;
each R^{c1} is independently H, -Q¹-NH—CO-Q²-T, a bioreversible group, or a non-bioreversible group;
each R^{A} is independently H or —OR^{C}, where R^{C} is -Q¹-NH₂, a bioreversible group, or a non-bioreversible group;
each R^{B} is independently H or —OR^{D}, where R^{D} is -Q¹-NH—CO-Q²-T, a bioreversible group, or a non-bioreversible group;
where
   each Q¹ is independently a divalent, trivalent, tetravalent, or pentavalent group, in which one valency is bonded to —NH—CO— or —NH₂, the second valency is open, and each of the remaining valencies, when present, is independently bonded to an auxiliary moiety;
   each Q² is independently a divalent, trivalent, tetravalent, or pentavalent group, in which one valency is bonded to —NH—CO—, the second valency is a bond to T, and each of the remaining valencies, when present, is independently bonded to an auxiliary moiety; and
   T is a linking moiety,
   provided that the starting material contains -Q¹-NH₂, and the product contains —Q¹-NH—CO-Q²-T.

On-support attachment:

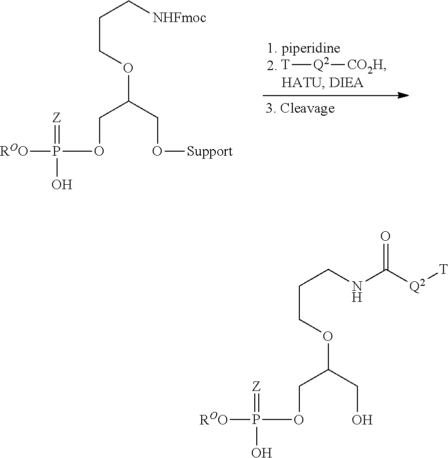

where
Z is O or S;
R^{O} is a bond to a nucleoside in an oligonucleotide;
each Q² is independently a divalent, trivalent, tetravalent, or pentavalent group, in which one valency is bonded to —NH—CO—, the second valency is a bond to T, and each of the remaining valencies, when present, is independently bonded to an auxiliary moiety; and
T is a linking moiety.

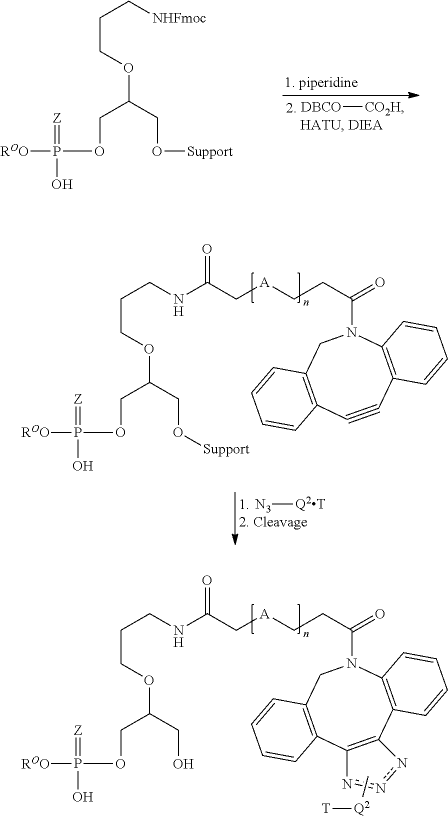

where
n is an integer from 1 to 8;
A is O or —CH₂—;
Z is O or S;
R^{O} is a bond to a nucleoside in an oligonucleotide;
each Q² is independently a divalent, trivalent, tetravalent, or pentavalent group; in which one valency is bonded to the azide or triazole, a second valency is bonded to T, and each of the remaining valencies, when present, is independently bonded to an auxiliary moiety; and
T is a linking moiety.

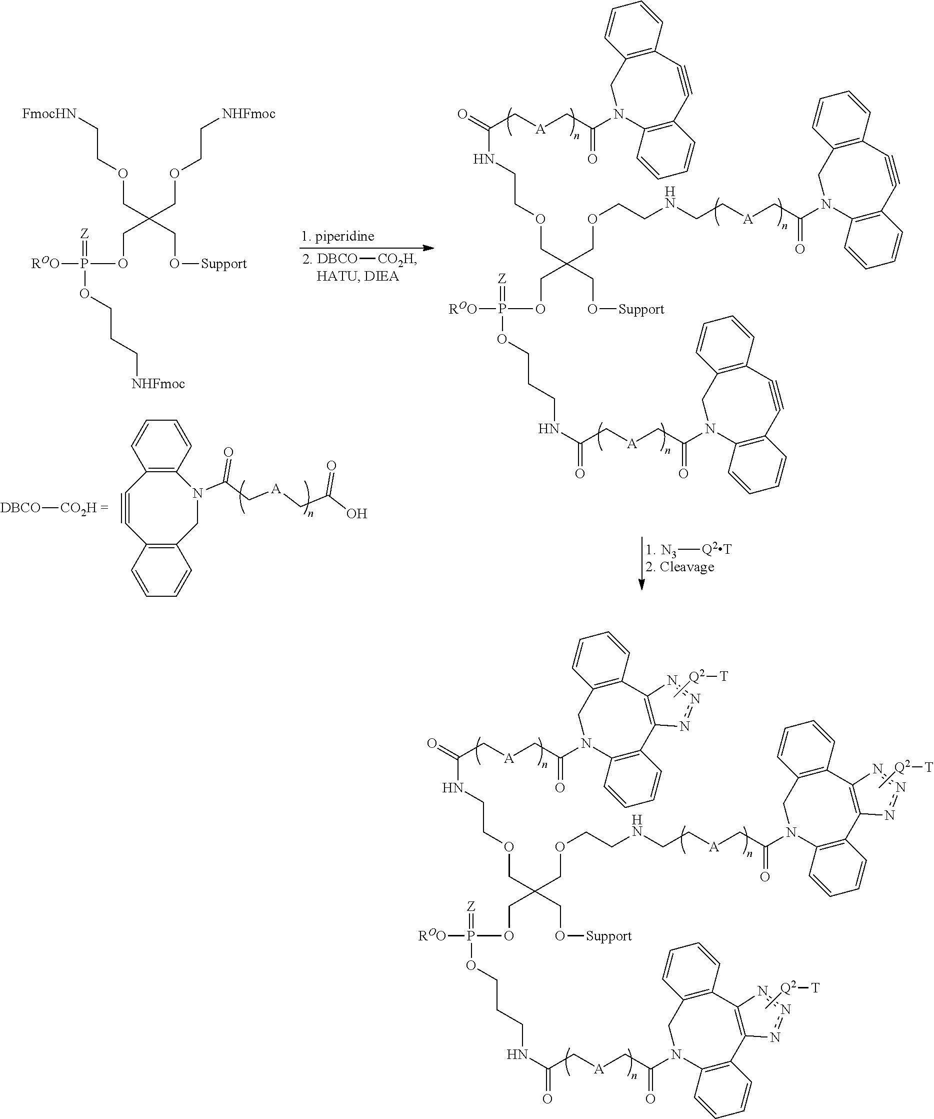

where
n is an integer from 1 to 8;
A is O or —CH₂—;
Z is O or S;
R^{O} is a bond to a nucleoside in an oligonucleotide;
each Q² is independently a divalent, trivalent, tetravalent, or pentavalent group; in which one valency is bonded to the azide or triazole, a second valency is bonded to T, and each of the remaining valencies, when present, is independently bonded to an auxiliary moiety; and
T is a linking moiety.

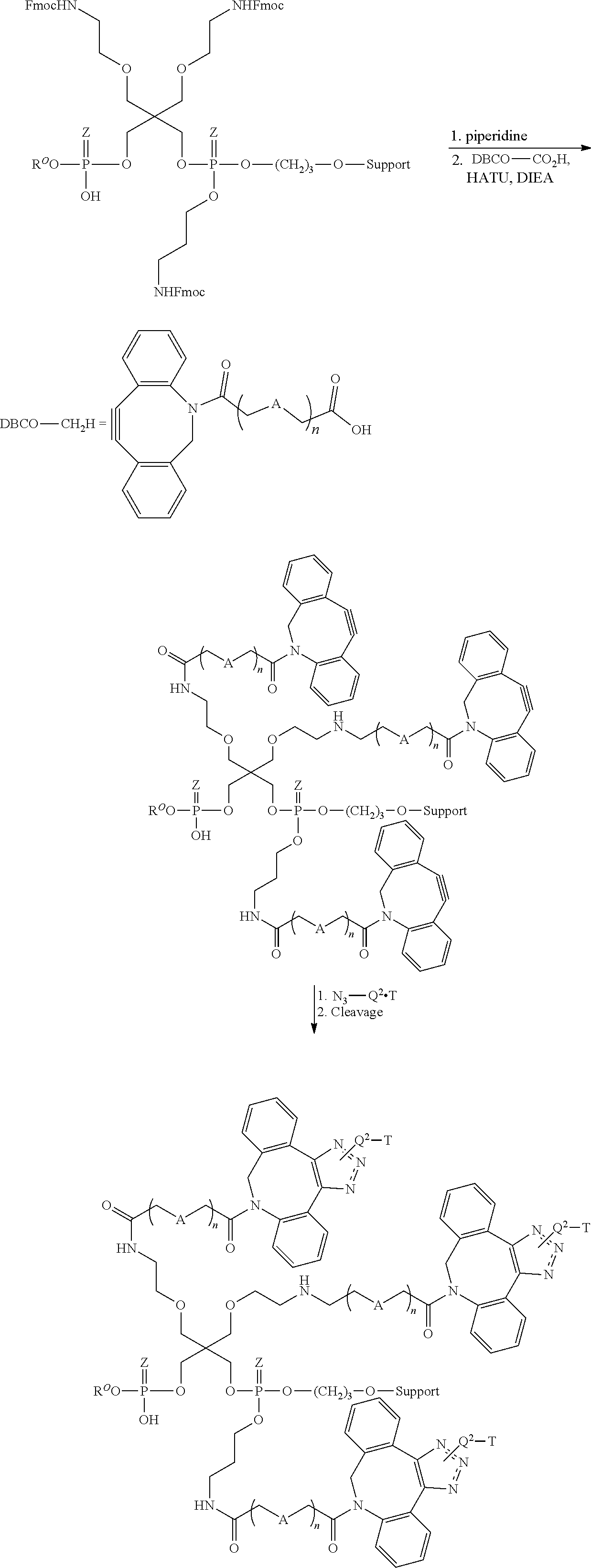

where
n is an integer from 1 to 8;
A is O or —CH₂—;
Z is O or S;
R^{O} is a bond to a nucleoside in an oligonucleotide;
each Q² is independently a divalent, trivalent, tetravalent, or pentavalent group; in which one valency is bonded to the azide or triazole, a second valency is bonded to T, and each of the remaining valencies, when present, is independently bonded to an auxiliary moiety; and
each T is independently a linking moiety.

### Representative Example of Fmoc Deprotection of a Phosphotriester:

An oligonucleotide including a phosphotriester with Fmoc-protected amine was subjected to deprotection conditions resulting in Fmoc deprotection without observable conversion of the phosphotriester into a phosphodiester.

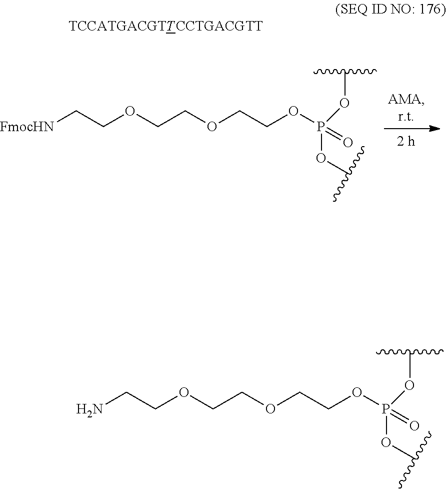

### DBCO-NHS Conjugation to TCCATGACGTTCCTGACGTT (SEQ ID NO:176)-Representative Example:

DBCO-NHS conjugation to the amino group in the phosphotriester was complete in 10 min at room temperature, as evidenced by mass spectrometric analysis.

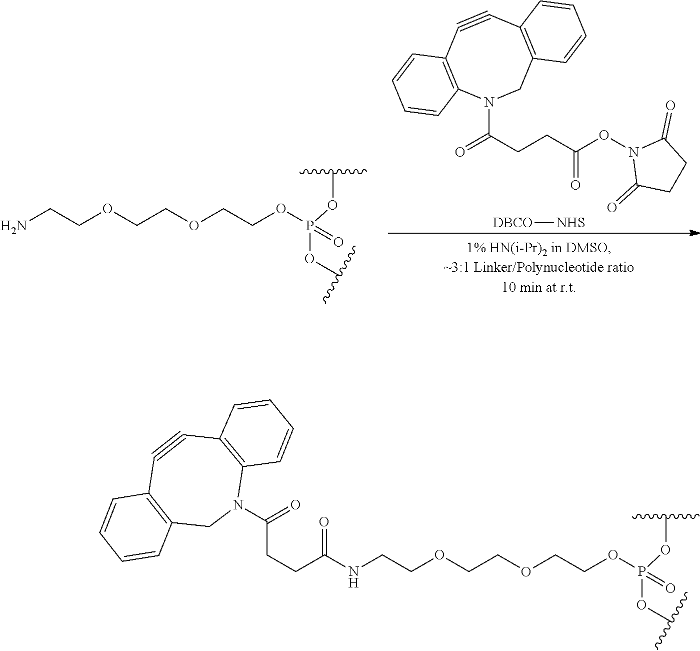

RP-HPLC purification of TCCATGACGTTCCTGACGTT (SEQ ID NO:176) containing a DBCO conjugating group was performed using the following conditions:
Buffer A=50 mM TEAA in Water;
Buffer B=90% Acetontrile; and
Flow Rate=1 mL/min;
Gradient:
   0-2 min (100% Buffer A/0% Buffer B),
   2-22 min (0% to 100% Buffer B), and
   22-25 min (100% Buffer B).

A similar procedure may be used to prepare an oligonucleotide using, e.g., 2′-modified nucleoside phosphoramidites, such as those described herein. Such a procedure is provided in International Patent application PCT/US2015/034749; the disclosure of the disulfide phosphotriester oligonucleotide synthesis in PCT/US2015/034749 is hereby incorporated by reference.

### V. Methods of Conjugation

Provided herein are methods for preparing a conjugate comprising a nectin-4 antibody or antigen-binding fragment thereof and one or more immunomodulating oligonucleotides linked via one or more Q-tag peptides as shown in the structure of Formula (A). In some embodiments, the methods comprise combining a nectin-4 antibody comprising at least one Q-tag peptide sequence with at least one exposed glutamine residue and an oligonucleotide under conditions sufficient to induce conjugation, i.e., amidation reaction between the CpG and Q tag. In other embodiments, the methods comprise reacting a nectin-4 antibody comprising at least one Q-tag peptide sequence with at least one exposed glutamine residue and an oligonucleotide under chemical conditions sufficient to induce conjugation. In still other embodiments, the methods comprise reacting a nectin-4 antibody comprising at least one Q-tag peptide sequence with at least one exposed glutamine residue and an oligonucleotide under enzymatic conditions, e.g., with transglutaminase, sufficient to induce conjugation.

### Transglutaminase-Mediated Conjugation Reaction Conditions

In one aspect, provided herein is a method of preparing a conjugate of formula (A), comprising combining one or more immunomodulating oligonucleotides (P) and a nectin-4 antibody comprising one or more glutamine residues. In one aspect, provided herein is a method of preparing a conjugate comprising a nectin-4 antibody or antigen-binding fragment (Ab) and one or more immunomodulating oligonucleotides (P), wherein the nectin-4 antibody or antigen-binding fragment is linked to one or more Q-tag peptides (Q) comprising the amino acid sequence RPQGF (SEQ ID NO:47), and wherein each immunomodulating oligonucleotide is linked to a Q-tag peptide via an amide bond with the glutamine residue of the Q-tag peptide and a linker (L) as shown in formula (A),

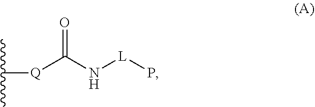

wherein:
[symbol] indicates the point of attachment of each Q to the nectin-4 antibody or antigen-binding fragment thereof (Ab);
each Q independently comprises a Q-tag peptide sequence RPQGF (SEQ ID NO:47);
each L is independently a bond or a linker moiety connected to Q via an amide bond with the glutamine residue; and
each P is independently an immunomodulating oligonucleotide; comprising contacting a compound of formula (B)

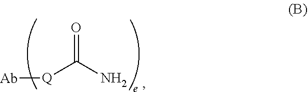

wherein Ab and Q are as defined for formula (A) above, and e is an integer from 1 to 20, with one or more immunomodulating oligonucleotides P, wherein each P independently has the following formula:

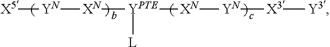

wherein
X^{5′} is a 5′ terminal nucleoside;
X^{3′} is a 3′ terminal nucleoside;
Y^{PTE} is an internucleoside phosphotriester;
Y³, is a terminal phosphotriester;
each X^{N} is independently a nucleoside;
each Y^{N} is independently an internucleoside linker;
b and c are each independently an integer from 1 to 25; with the proviso that the sum of b and c is at least 5; and
L is a linker moiety comprising a terminal amine, in the presence of a transglutaminase.

In another aspect, method for preparing a conjugate comprising a nectin-4 antibody or antigen-binding fragment (Ab) and one or more immunomodulating oligonucleotides (P), wherein the nectin-4 antibody or antigen-binding fragment is linked to one or more Q-tag peptides (Q) comprising at least one glutamine residue, and wherein each immunomodulating oligonucleotide is linked to a Q-tag peptide via an amide bond with the glutamine residue of the Q-tag peptide and a linker (L) as shown in Formula (A),

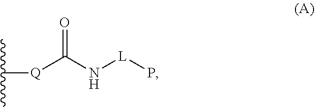

wherein:
[symbol] indicates the point of attachment of each Q to the nectin-4 antibody or antigen-binding fragment thereof (Ab);
each Q is independently a Q-tag peptide comprising at least one glutamine residue;
each L is independently a bond or a linker moiety connected to Q via an amide bond with the glutamine residue; and
each P is independently an immunomodulating oligonucleotide; comprising contacting a compound of formula (B)

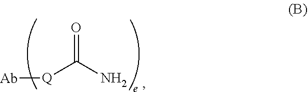

wherein Ab and Q are as defined for formula (A) above, and e is an integer from 1 to 20, with one or more immunomodulating oligonucleotides P, wherein each oligonucleotide P is independently an immunomodulating oligonucleotide of formula (C) or formula (D), in the presence of a transglutaminase.

In some embodiments, the conjugate comprises one or more, two or more, three or more, four or more, five or more, six or more, seven or more, eight or more, nine or more, ten or more, or twenty or more Q-tag peptides. In some embodiments, the conjugate comprises one, two, three, four, five, six, seven, eight, nine, ten, or twenty Q-tag peptides. In some embodiments, the conjugate has 2 Q-tag peptides. In some embodiments, the conjugate comprises one or more, two or more, three or more, four or more, five or more, six or more, seven or more, eight or more, nine or more, ten or more, or twenty or more immunomodulating oligonucleotides. In some embodiments, the conjugate comprises one, two, three, four, five, six, seven, eight, nine, ten, or twenty immunomodulating oligonucleotides. In some embodiments, the conjugate has one immunomodulating oligonucleotide.

In another aspect, the method comprises combining a compound of Formula (C) and a nectin-4 antibody of formula (B) comprising one or more glutamine residues in the presence of a transglutaminase. In some embodiments, the method comprises contacting a compound of Formula (D) and a nectin-4 antibody of formula (B) comprising one or more glutamine residues in the presence of a transglutaminase. In some embodiments, the final concentration of the compound of Formula (C) or Formula (D) is in the range of about 1-100 PM. In some embodiments, the final concentration of the Q tag comprising antibody is in the range of about 1-500 PM. In some embodiments, the final concentration of transglutaminase is in the range of about 1-500 PM. In some embodiments, the final concentration of transglutaminase is in the range of about 1-50 μM, about 50-100 μM, about 100-150 μM, about 150-200 μM, about 200-250 μM, about 250-300 μM, about 300-400 μM, about 400-500 μM, about 100-125 μM, about 125-150 PM, about 150-175 μM, about 175-200 μM, about 200-225 μM, about 225-250 μM, about 250-275 μM, about 275-300 μM, about 300-325 μM or about 325-350 μM.

In some embodiments, the ratio of the Q tag comprising antibody and the compound of Formula (C) or Formula (D) is in the range of about 1:1-250:1, about 1:1-5:1, about 5:1-10:1, about 10:1-20:1, about 20:1-30:1, about 30:1-40:1, about 40:1-50:1, about 50:1-75:1, about 75:1-100:1, about 100:1-150:1, about 150:1-200:1, about 200:1-250:1, about 1:1-25:1, about 25:1-50:1, about 50:1-75:1, about 75:1-100:1 or about 100:1-250:1 by weight. In some embodiments, the ratio of the compound of Formula (C) or Formula (D) and the antibody is about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1 or about 20:1 by molarity.

In some embodiments, the ratio of the Q tag comprising antibody and transglutaminase is in the range of about 1:1-500:1, about 1:1-5:1, about 5:1-10:1, about 10:1-20:1, about 20:1-30:1, about 30:1-40:1, about 40:1-50:1, about 50:1-75:1, about 75:1-100:1, about 100:1-150:1, about 150:1-200:1, about 200:1-250:1, about 1:1-25:1, about 25:1-50:1, about 50:1-75:1, about 75:1-100:1, about 100:1-150:1, about 150:1-200:1, about 200:1-250:1, about 250:1-300:1, about 300:1-400:1 or about 400:1-500:1 by weight. In some embodiments, the ratio of the peptide and transglutaminase is about 15:1, about 16:1, about 17:1, about 18:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 37:1, about 38:1, about 39:1, about 40:1, about 41:1, about 42:1, about 43:1, about 44:1, about 45:1, about 46:1, about 47:1, about 48:1, about 49:1 or about 50:1 by molarity.

In some embodiments, the ratio of Q tag: CpG: transglutaminase is about 1:1.3:10. In some embodiments, the ratio of Q tag: CpG: transglutaminase is about 1:1.5:10. In some embodiments, the ratio of Q tag: CpG: transglutaminase is about 1:1.3:15.

In some embodiments, the reaction is incubated at greater than 15° C., greater than 20° C., greater than 25° C., greater than 30° C., greater than 35° C., greater than 40° C., greater than 45° C., or greater than 50° C. In some embodiments, the reaction is incubated at about room temperature. In some embodiments, the reaction is incubated for at least 10 minute, 20 minutes, 30 minutes, 45 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours or 60 hours.

In some embodiments, the method described herein produces the compound of Formula (A) at greater than about 5%, greater than about 10%, greater than about 15%, greater than about 20%, greater than about 25%, greater than about 30%, greater than about 35%, greater than about 40%, greater than about 45%, greater than about 50%, greater than about 60%, greater than about 70%, greater than about 80%, greater than about 90%, greater than about 95%, greater than about 97% or greater than about 99% as compared to the peptide.

In some embodiments, the pH of the reaction is in the range of about 4-10. In some embodiments, the pH of the reaction is in the range of about 4-6, about 6-8 or about 8-10. In some embodiments, the pH of the reaction is in the range of about 7-8.

In another aspect, reactions useful for attaching a linking moiety to an oligonucleotide are known in the art, including, but not limited to Hiiisgen cycloaddition (metal-catalyzed or metal-free) between an azido and an alkyne-based conjugating group (e.g., optionally substituted C₆₋₁₆ heterocyclylene containing an endocyclic carbon-carbon triple bond or optionally substituted C₈₋₁₆ cycloalkynyl) to form a triazole moiety; the Diels-Alder reaction between a dienophile and a diene/hetero-diene; bond formation via pericyclic reactions such as the ene reaction; amide or thioamide bond formation; sulfonamide bond formation (e.g., with azido compounds); alcohol or phenol alkylation (e.g., Williamson alkylation), condensation reactions to form oxime, hydrazone, or semicarbazide group; conjugate addition reactions by nucleophiles (e.g., amines and thiols); disulfide bond formation; and nucleophilic substitution (e.g., by an amine, thiol, or hydroxyl nucleophile) at a carbonyl (e.g., at an activated carboxylic acid ester, such as pentafluorophenyl (PFP) ester or tetrafluorophenyl (TFP) ester) or at an electrophilic arene (e.g., SNAr at an oligofluorinated arene, a fluorobenzonitrile group, or fluoronitrobenzene group).

In certain embodiments, the attachment reaction is a dipolar cycloaddition, and the conjugation moiety includes azido, optionally substituted C₆₋₁₆ heterocyclylene containing an endocyclic carbon-carbon triple bond, or optionally substituted C₈₋₁₆ cycloalkynyl. The complementary reactive group and the conjugating group are selected for their mutual complementarity. For example, an azide is used in one of the conjugating group and the complementary reactive group, while an alkyne is used in the other of the conjugating group and the complementary reactive group.

### Attachment of Linking Moiety to the Oligonucleotide

A linking moiety can be attached to an oligonucleotide by forming a bond between a attaching group in the oligonucleotide and a complementary reactive group bonded to the linking moiety. In certain embodiments, the linking moiety, is modified to include a complementary reactive group. Methods of introducing such complementary reactive groups into a linking moiety is known in the art.

In certain embodiments, the complementary reactive group is optionally substituted C₂₋₁₂ alkynyl, optionally substituted N-protected amino, azido, N-maleimido, S⁻protected thiol,

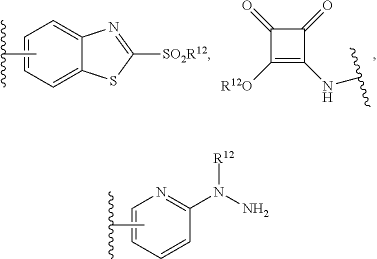

or a N-protected moiety thereof,

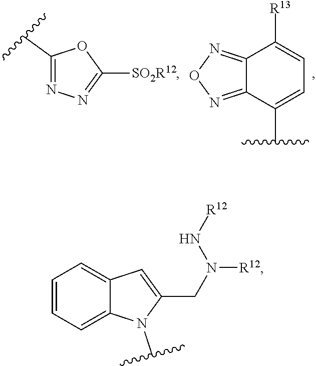

optionally substituted C₆₋₁₆ heterocyclyl containing an endocyclic carbon-carbon triple bond (e.g.,

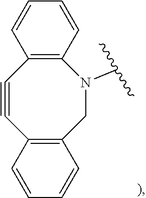 [SMILES: *N1Cc2ccccc2C#Cc2ccccc21]

1,2,4,5-tetrazine group (e.g.,

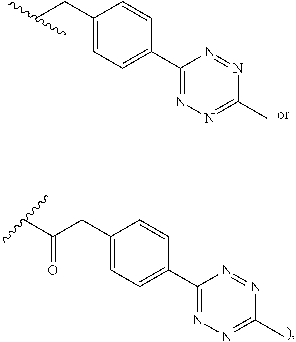 [SMILES: *Cc1ccc(-c2nnc(C)nn2)cc1 ; *C(=O)Cc1ccc(-c2nnc(C)nn2)cc1]

optionally substituted C₈₋₁₆ cycloalkynyl (e.g.,

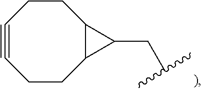 [SMILES: *CC1C2CCC#CCCC12]

—NHR^{N1}, optionally substituted C₄-8 strained cycloalkenyl (e.g., trans -cyclooctenyl or norbornenyl), or optionally substituted C₁₋₁₆ alkyl containing —COOR¹² or —CHO;
wherein:
R^{N}1 is H, N-protecting group, or optionally substituted C₁₋₆ alkyl;
each R¹² is independently H, optionally substituted C₁₋₆ alkyl, or O⁻protecting group (e.g., a carboxyl protecting group); and
R¹³ is halogen (e.g., F).

In certain embodiments, the complementary reactive group is protected until the conjugation reaction. For example, a complementary reactive group that is protected can include —COOR^{PGO} or —NHR^{PGN}, where R^{PGO} is an O⁻protecting group (e.g., a carboxyl protecting group), and R^{PGN} is an N-protecting group.

### VI. Pharmaceutical Compositions

The nectin-4 antibodies and conjugates of the present invention, such as the conjugates comprising structures of formula (A), antibodies of formula (B), and immunomodulating oligonucleotides of formulae (C), (C′), (C″), (D), (D′) and (D″), or a pharmaceutically acceptable salt of any of the foregoing, or any subgroup thereof may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form, as the active ingredient is combined in admixture with a pharmaceutically acceptable carrier, which carrier may take a wide variety of forms depending on the form of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, lubricants, binders, disintegrating agents and the like in the case of powders, pills, capsules, and tablets. Because of their ease in administration, tablets and capsules represent the most advantageous oral dosage unit forms, in which case solid pharmaceutical carriers are employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. The compounds of the present invention may also be administered via oral inhalation or insufflation in the form of a solution, a suspension or a dry powder using any art-known delivery system.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, suppositories, powder packets, wafers, injectable solutions or suspensions and the like, and segregated multiples thereof.

Administration can be, but is not limited to, intravenous, intraarterial, subcutaneous, intraperitoneal, subdermal (e.g., via an implanted device), and intraparenchymal administration. In some embodiments, the pharmaceutical compositions described herein are administered by subcutaneous injection.

The pharmaceutical compositions including a conjugate described herein can be delivered to a cell, group of cells, tumor, tissue, or subject using delivery technologies known in the art. In general, any suitable method recognized in the art for delivering a nucleic acid-protein conjugate (in vitro or in vivo) can be adapted for use with a herein described compositions. For example, delivery can be by local administration, (e.g., direct injection, implantation, or topical administering), systemic administration, or subcutaneous, intravenous, intraperitoneal, or parenteral routes, including intracranial (e.g., intraventricular, intraparenchymal and intrathecal), intramuscular, transdermal, airway (aerosol), nasal, oral, rectal, or topical (including buccal and sublingual) administration. In certain embodiments, the compositions are administered by subcutaneous or intravenous infusion or injection.

Accordingly, in some embodiments, the herein described pharmaceutical compositions may comprise one or more pharmaceutically acceptable excipients. In some embodiments, the pharmaceutical compositions described herein can be formulated for administration to a subject.

As used herein, a pharmaceutical composition or medicament includes a pharmacologically effective amount of at least one of the described therapeutic compounds or conjugates and one or more pharmaceutically acceptable excipients. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

The compound or conjugate can be formulated in compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the therapeutic effect to be achieved, and the limitations inherent in the art of compounding such an active compound for the treatment of individuals.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.).

Generally, an effective amount of an active compound will be in the range of from about 0.1 to about 100 mg/kg of body weight/day, e.g., from about 1.0 to about 50 mg/kg of body weight/day. In some embodiments, an effective amount of an active compound will be in the range of from about 0.25 to about 5 mg/kg of body weight per dose. In some embodiments, an effective amount of an active compound will be in the range of 25-400 mg per 1-18 weeks or 1-6 months. In some embodiments, an effective amount of an active compound will be in the range of 50-125 mg per 4 weeks or per one month. In some embodiments, an effective amount of an active ingredient will be in the range of from about 0.5 to about 3 mg/kg of body weight per dose. In some embodiments, an effective amount of an active ingredient will be in the range of from about 25-400 mg per dose. In some embodiments, an effective amount of an active ingredient will be in the range of from about 50-125 mg per dose. The amount administered will also likely depend on such variables as the overall health status of the patient, the relative biological efficacy of the compound delivered, the formulation of the drug, the presence and types of excipients in the formulation, and the route of administration. Also, it is to be understood that the initial dosage administered can be increased beyond the above upper level in order to rapidly achieve the desired blood-level or tissue level, or the initial dosage can be smaller than the optimum.

For treatment of disease or for formation of a medicament or composition for treatment of a disease, the pharmaceutical compositions described herein including a nectin-4 antibody or conjugate can be combined with an excipient or with a second therapeutic agent or treatment including, but not limited to: a second or other conjugates, a small molecule drug, an antibody, an antibody fragment, and/or a vaccine.

The described nectin-4 antibodies or conjugates, when added to pharmaceutically acceptable excipients or adjuvants, can be packaged into kits, containers, packs, or dispensers. The pharmaceutical compositions described herein may be packaged in pre-filled syringes or vials.

### VII. Kits

Also provided herein is a kit comprising a conjugate as described above.

In another aspect, the kit further comprises a package insert including, without limitation, appropriate instructions for preparation and administration of the formulation, side effects of the formulation, and any other relevant information. The instructions may be in any suitable format, including, but not limited to, printed matter, videotape, computer readable disk, optical disc or directions to internet-based instructions.

In another aspect, kits for treating an individual who suffers from or is susceptible to the conditions described herein are provided, comprising a first container comprising a dosage amount of a composition or formulation as disclosed herein, and a package insert for use. The container may be any of those known in the art and appropriate for storage and delivery of intravenous formulation. In certain embodiments, the kit further comprises a second container comprising a pharmaceutically acceptable carrier, diluent, adjuvant, etc. for preparation of the formulation to be administered to the individual.

In another aspect, kits may also be provided that contain sufficient dosages of the compositions described herein (including pharmaceutical compositions thereof) to provide effective treatment for an individual for an extended period, such as 1-3 days, 1-5 days, a week, 2 weeks, 3, weeks, 4 weeks, 6 weeks, 8 weeks, 1 cycle, 2 cycles, 3 cycles, 4 cycles, 5 cycles, 6 cycles, 7 cycles, 8 cycles or more.

In some embodiments, the kits may also include multiple doses and may be packaged in quantities sufficient for storage and use in pharmacies, for example, hospital pharmacies and compounding pharmacies. In certain embodiments the kits may include a dosage amount of at least one composition as disclosed herein.

### VIII. Methods of Treatment

Also provided herein are methods for treating a disease or disorder in a subject comprising administering an effective amount of a nectin-4 antibody or conjugate described herein to the subject in need thereof. Also provided herein are uses of a nectin-4 antibody or conjugate described herein in the preparation of a medicament for treating a patient in need of treatment with the oligonucleotide in the conjugate. Also provided are nectin-4 antibodies or conjugates as described herein for treating a disease or disorder in a subject in need of the treatment with the oligonucleotide in the nectin-4 antibodies or conjugates. Also provided are nectin-4 antibodies or conjugates as described herein for treating a patient comprising administering an effective amount of the nectin-4 antibody or conjugate to the patient. In some embodiments, the subject has or at the risk of developing cancer. In some embodiments, the disease or disorder is a viral infection. In some embodiments, the disease is caused by disorder of immune systems.

In some embodiments of the methods of treating cancer as described herein, the cancer being treated with the methods disclosed herein is a solid tumor. In some embodiments, the cancer being treated with the methods disclosed herein is a liquid tumor. In some embodiments, the cancer being treated with the methods disclosed herein is a solid tumor. In particular embodiments, the cancer being treated with the methods disclosed herein is breast cancer, colorectal cancer, lung cancer, head and neck cancer, melanoma, lymphoma, or leukemia. In some embodiments, cancers include, but are not limited to, multiple myeloma, Waldenström's macroglobulinemia, the heavy chain diseases, such as, for example, alpha chain disease, gamma chain disease, and mu chain disease, benign monoclonal qammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, sominoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated. In particular embodiments, the cancer being treated with the methods disclosed herein is selected from the list consisting of mantle cell cymphoma (MCL), diffuse large B-cell lymphoma (DLBCL), Burkitts lymphoma, multiple melanoma (MM), chronic lymphocytic leukemia (CLL), acute myeloid leukemia (AML), small lymphocytic lymphoma (SLL), hairy cell leukemia (HCL), lymphoplasmacytic lymphoma (LPL), skeletal muscle lymphoma (SML), splenic marginal zone lymphoma (SMZL), follicle center lymphoma (FCL), colorectal cancer, non-small cell lung cancer (NSCLC), head and neck cancer, breast cancer, pancreatic cancer, glioblastoma (GBM), prostate cancer, esophageal cancer, renal cell carcinoma, hepatic carcinoma, bladder cancer and gastric carcinoma.

In some embodiments, provided herein methods for treating a disease or disorder in a subject comprising administering an effective amount of a CpG-Ab immunoconjugate described herein to the subject in need thereof, wherein the CpG-Ab immunoconjugate binds to nectin-4, such as a CpG-Ab immunoconjugate comprising a nectin-4 antibody or antigen binding fragment thereof, and where the disease or disorder treated is a cancer characterized by nectin-4 overexpression. In some embodiments, such cancers include esophageal cancer, stomach cancer, breast cancer, ovarian cancer, lung cancer, pancreatic adenocarcinoma, colon carcinoma, bladder cancer, cervical cancer, thyroid cancer, uterine cancer, rectal cancer, and gallbladder cancer. In some embodiments, the CpG-Ab immunoconjugate comprises the sequence or structures of oligonucleotides listed in one of Tables 2-6.

In some embodiments, the methods of treatment include administration of a CpG-Ab immunoconjugate that binds to nectin-4 present on a tumor cell (i.e., CpG-nectin-4-Ab immunoconjugate) and the treatment results in the killing of or impairment of tumor cell(s) such that the volume, size and/or growth of the tumor is reduced or inhibited. In some embodiments, provided herein are methods for treating a disease or disorder in a subject comprising administering an effective amount of a CpG-Ab immunoconjugate described herein to the subject in need thereof, wherein the CpG-Ab immunoconjugate binds to nectin-4, such as a CpG-Ab immunoconjugate comprising a nectin-4 antibody or antigen binding fragment thereof, and where the disease or disorder treated is a cancer characterized by nectin-4-expressing tumor cells. In some embodiments, the disease or disorder treated is a cancer where tumor cells over-express nectin-4. In some embodiments, the disease or disorder treated is a cancer where tumor cells have low levels of nectin-4 expression. In some embodiments, the disease or disorder treated is a cancer where tumor cells have moderate levels of nectin-4 expression. In some embodiments, the disease or disorder treated is a cancer where tumor cells are not responsive or have a low responsiveness to another nectin-4 directed therapy (such as a nectin-4 antibody-small molecule drug conjugate). In some embodiments, the disease or disorder treated is a cancer where tumor cells have detectable and low to moderate levels of nectin-4 expression. In some embodiments, such cancers include skin melanoma, sarcoma, kidney cancer, glioblastoma, mesothelioma, thymoma, liver hepatocellular carcinoma, pheochromocytoma and paraganglioma, and lower grade glioma.

In some embodiments, the cancer being treated with the methods disclosed herein is resistant to at least one immunotherapy. In some embodiments, the cancer being treated with the methods disclosed herein is resistant to at least one cancer therapy selected from the group consisting of chemotherapy, radiation, targeted therapy, vaccine therapy, and CAR-T therapy. In some embodiments, the method of treating cancer comprises co-administering to a subject having cancer (i) a therapeutically effective amount of the CpG-nectin-4-Ab immunoconjugate; and (ii) the immunotherapeutic agent which the cancer being treated has shown to resist or not to respond, when the cancer is treated with the immunotherapeutic agent alone.

In some embodiments, the methods of treatment described herein include administration of a CpG-Ab immunoconjugate that binds to nectin-4 (CpG-nectin-4-Ab immunoconjugate) in combination with one or more other cancer therapies. In some embodiments, the one or more other cancer therapies can be immunotherapy, cancer therapeutics (including, but not limited to, antibodies and/or small molecule inhibitors), cell therapy, a cancer vaccine, and chemotherapy.

In particular embodiments, the methods of treatment provided herein include treatment of a subject who has been previously treated or concurrently treated with an immune checkpoint modulator. In some embodiments, the immune checkpoint modulator is a PD-1 inhibitor. In some embodiments, the immune checkpoint modulator is a PD-L1 inhibitor. In particular, in some embodiments, the inhibitor of PD-1 is an anti-PD-1 antibody or an antigen-binding fragment thereof. In some embodiments, the inhibitor of PD-L1 is an anti-PD-L1 antibody or an antigen-binding fragment thereof. In some embodiments, the tumor or tumor cells of the treated subject are responsive to the immune checkpoint modulator, and treatment with a CpG-nectin-4-Ab immunoconjugate improves the responsiveness of the tumor or tumor cells to the treatment. In some cases, the improvement is a reduction in the size of the tumor, growth of the tumor or tumor cells and/or a reduction in metastasis of tumor cells.

In some embodiments, the cancer to be treated is responsive (i.e., can be treated) by immune checkpoint inhibitor therapy. In some embodiments, the cancer is not response (i.e., is refractory to or resistant to) immune checkpoint inhibitor therapy. In some embodiments, the immune checkpoint inhibitor therapy comprises a PD-1 inhibitor, such as an anti-PD-1 antibody. In some embodiments, the immune checkpoint inhibitor therapy comprises a PD-L1 inhibitor, such as an anti-PD-L1 antibody. In some embodiments, the immune checkpoint inhibitor therapy comprises a CTLA-4 inhibitor, such as an anti-CTLA-4 antibody.

In particular embodiments, the tumor or tumor cells of a treated subject are not responsive or are less responsive to an immune checkpoint modulator (such as an inhibitor) and treatment with a CpG-nectin-4-Ab immunoconjugate improves the responsiveness of the tumor or tumor cells to the treatment. In particular embodiments, the immune checkpoint modulator is an inhibitor of PD-1. In particular embodiments, the immune checkpoint modulator is an inhibitor of PD-L1. In some embodiments, the method of treating cancer comprises co-administering to a subject having cancer (i) a therapeutically effective amount of a CpG-nectin-4-Ab immunoconjugate; and (ii) a therapeutically effective amount of the inhibitor of PD-1. In some embodiments, the method of treating cancer comprises co-administering to a subject having cancer (i) a therapeutically effective amount of a CpG-nectin-4-Ab immunoconjugate; and (ii) a therapeutically effective amount of the inhibitor of PD-L1. In particular, in some embodiments, the inhibitor of PD-1 is an anti-PD-1 antibody or an antigen-binding fragment thereof. In some embodiments, the inhibitor of PD-L1 is an anti-PD-L1 antibody or an antigen-binding fragment thereof. In some embodiments, the treatment is directed to a subject that does not respond to or is resistant to a PD-1 or PD-L1 inhibitor and such subject is treated with a CpG-Ab immunoconjugate that binds nectin-4, such as a CpG-Ab immunoconjugate comprising a nectin-4 antibody or antigen binding fragment thereof. In some cases, the response of the tumor or tumor cells to the co-administration treatment is greater than the response to either single agent administered alone.

Exemplary PD-L1 inhibitors are known in the art and include, without limitation, atezolizumab (TECENTRIQ®; Genentech), avelumab (BAVENCIO®; EMD Serono), durvalumab (IMFINZI®; AstraZeneca), KN035, CK-301, AUNP12, CA-170, and BMS-986189. Exemplary PD-1 inhibitors are known in the art and include, without limitation, pembrolizumab (KEYTRUDA®; Merck), nivolumab (OPDIVO®; Bristol Myers Squibb), cemiplimab-rwlc (LIBTAYO®; Regeneron/Sanofi), dostarlimab-gxly (JEMPERLI®; GlaxoSmithKline), JTX-4014, spartalizumab (PDR001), camrelizumab (SHR1210), sintilimab (IB1I308), tislelizumab (BGB-A317), toripalimab (JS 001), INCMGA00012, AMP-224, and AMP-514.

In some embodiments, the cancer being prevented or treated using the methods provided herein is an episode of cancer recurrence in a subject who is in partial or complete remission of a prior cancer. In particular embodiments, the prior cancer is a liquid cancer (e.g., blood cancer) and the recurrent cancer being prevented or treated is a liquid tumor. In particular embodiments, the prior cancer is a solid tumor cancer and the recurrent cancer being prevented or treated is a solid tumor. In particular embodiments, the prior cancer is a liquid tumor cancer and the recurrent cancer being prevented or treated is a solid tumor cancer. In particular embodiments, the prior cancer is a solid tumor cancer and the recurrent cancer being prevented or treated is a liquid tumor cancer.

In some embodiments, the cancer being prevented or treated using the methods provided herein is first episode of cancer recurrence in the subject after the subject showed partial or complete remission. In some embodiments, the cancer being prevented or treated using the methods provided herein is second episode of cancer recurrence in the subject after the subject showed partial or complete remission. In some embodiments, the cancer being prevented or treated using the methods provided herein is third episode of cancer recurrence in the subject after the subject showed partial or complete remission. In some embodiments, the cancer being prevented or treated using the methods provided herein is an episode of cancer recurrence subsequent to the third episode of cancer recurrence in the subject after the subject showed partial or complete remission.

In some embodiments of the methods and uses described herein, wherein upon administration of the CpG-nectin-4-Ab immunoconjugate, the CpG-containing immunostimulating oligonucleotide specifically activates a toll-like receptor 9 (TLR9), such as in a myeloid cell. In some embodiments of the methods and uses described herein, the administration of the CpG-nectin-4-Ab immunoconjugate results in one or more of dendritic cell activation, monocyte activation, enhancement of tumor phagocytosis by macrophages, and/or enhancement of cytokine secretion.

In some embodiments of the methods and uses described herein, administration of the CpG-nectin-4-Ab immunoconjugate to an individual activates immune cells in the individual. In some embodiments, the immune cells are T cells and/or NK cells. In some embodiments, the immune cells are T cells. In some embodiments, the immune cells are NK cells. In some embodiments, the immune cells are T cells and NK cells.

Also provided herein are methods of activating immune cells in an individual, comprising administering an effective amount of the CpG-nectin-4-Ab immunoconjugate described herein to the individual. Also provided herein are uses of the CpG-nectin-4-Ab immunoconjugate described herein in the preparation of a medicament for activating immune cells in an individual. In some embodiments, the immune cells are T cells and/or NK cells. In some embodiments, activating immune cells in the individual treats a cancer in the individual. In some embodiments, the cancer is selected from any of the cancers described above.

Without being bound to theory, nectin-4 antibody oligonucleotide conjugate's binding to nectin-4 on tumor cells and enrichment of nectin-4 antibody oligonucleotide conjugates in the tumor microenvironment, results in ligand-induced engagement of Fc gamma receptors on myeloid cells including dendritic cells, triggers Fc clustering followed by Fc-mediated signaling and internalization. Following internalization, the trafficking of the immunoglobulin complex to the endosome activates TLR9, triggering the secretion of pro-inflammatory cytokines. In dendritic cells, that internalization of immunoglobulin complexes with target may induce processing and presentation of tumor peptides. Nectin-4 binding on tumor cells may also induce phagocytosis of target cells, which can also lead to the internalization of the nectin-4 antibody-oligonucleotide conjugates, triggering TLR9 activation in the endosome. Therefore, co-engagement of tumor antigen and FcgR promote delivery of TLR9 agonist into myeloid cells and its activation.

### EXAMPLES

The presently disclosed subject matter will be better understood by reference to the following Examples, which are provided as exemplary of the invention, and not by way of limitation.

### Materials

Prototype peptides were made in house, but can be purchased at custom peptide suppliers (e.g., CPC Scientific). Oligonucleotides were made in-house or by LGC. Transglutaminase used in these examples were isolated from various bacterial Streptoverticillium strain (e.g., Ajinomoto). The Q-tag mAbs were produced at Sino Biologicals or internally.

### Production of Oligonucleotides

Oligonucleotides were generally prepared in accordance with the solid phase synthesis scheme shown below, beginning with an initial deprotection of the solid support for the oligonucleotide synthesis, followed by coupling of the solid support with to the first nucleotide, thiolation to give the phosphothioester and repeated deprotection and coupling to give the entire oligonucleotide sequence.

The general synthesis of oligonucleotides as provided herein is described below.

Deprotection: A dimethoxytrityl-1,3-propanediol glycolate protected controlled pore glass solid support (DMTO-C₃-CPG, 1000 Å, Bulk Density 0.26-0.36 g/cc, Loading 30-40 μmol/g) was reacted with 3% dichloroacetic acid in toluene (v/v) at 25° C., to give the deprotected CPG support. UV absorption of an aliquot of the reaction mixture was measured to identify the reaction endpoint (wavelength 350 nm, target minimum absorbance 0.25 OD, using a fixed watch command setting) and to confirm removal of the dimethoxytrityl protecting group.

Activation/Coupling: The deprotected CPG support was coupled with the first nucleotide phosphoramidite precursor for the 3′-end, for the respective oligonucleotide to be synthesized, by adding and mixing the desired 3′ nucleotide (3 equiv.) for 5 minutes at 25° C. to the reactor containing the deprotected CPG support in the presence of an activator 5-Ethylthio-1H-tetrazole (0.5M in ACN) at 60% of the nucleotide concentration.

Thiolation/Sulfurization: Following the coupling step, the linking phosphite triester moiety of the added nucleotide precursor was thiolated (or sulfurized) by adding Polyorg Sulfa (3-phenyl 1,2,4-dithiazoline-5-one), 0.15M in dry ACN, to give the phosphothioester.

Capping: After sulfurization, the CPG support and linked nucleotide were treated with two capping compositions (Capping composition A: 20% N-methylimidazole in ACN; Capping B composition B: 20% Acetic Anhydride, 30% Pyridine, 50% ACN) to block unreacted nucleotide reactants.

Repeat Synthesis: The remaining nucleotides were added in sequence from the 3′ end to the 5′ end, employing the appropriate phosphoramidite precursors in solution, by repeating the steps of deprotection, activation/coupling, thiolation/sulfurization and capping as described above to obtain the desired oligonucleotide sequence in protected form. All phosphoramidite prescursors were mixed with the CPG support for 5 minutes during the coupling step, except for dT-Thiophosphoramidite, which was mixed for 15 minutes.

Selected phosphoramidite precursors used in the synthesis are shown below. The phosphoramidite precursors were prepared in solutions with the solvents and at the concentrations, respectively shown, to be used in the coupling steps.

| Amidite | Structure | Concentration |
|---|---|---|
| DMT-dC(Ac) Amidite | 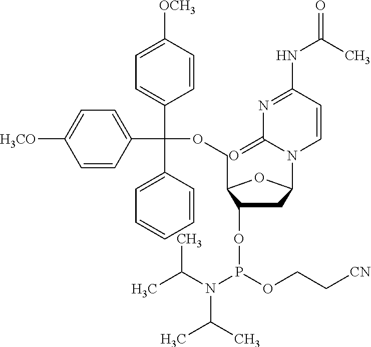 | 0.1M in dry ACN: |
| DMT-dG(dmf) Amidite | 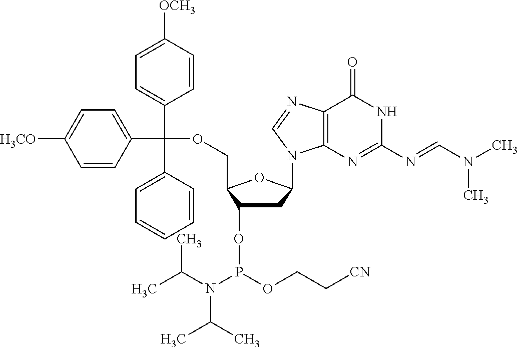 | 0.1M in dry ACN: |
| DMT-dT phosphoramidite | 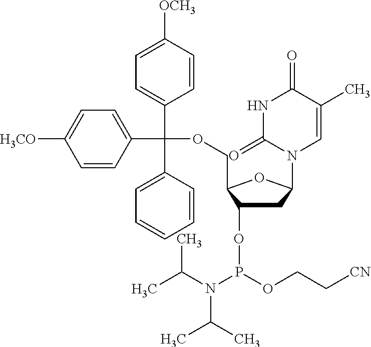 | 0.1M in dry ACN: |
| Fmoc-protected DMT- dT PEG2 NH2 Amidite | 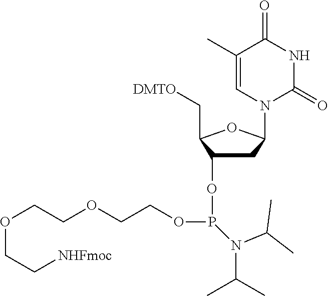 | 0.1M in dry ACN |
| 5-Br-dU-CE | | 0.1M in dry ACN |
| Phosphoramidite | | |
| dT- Thiophsophoramidite | 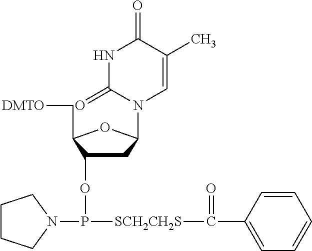 | 0.15M in dry 10% (v/v) DCM/ACN |
| 2′-O-Methyl 5-Methyl | | 0.1M in dry ACN |
| Uridine CED | | |
| phosphoramidite | | |
| dG- Thiophosphoramidite | 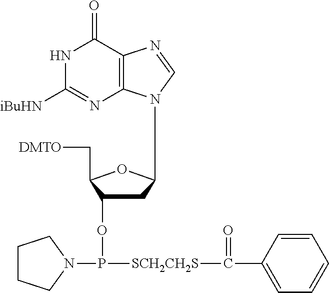 | 0.1M in dry ACN |

Exemplary Fmoc-protected oligonucleotide compounds 6.1a, 6.2a and 6.3a obtained from the synthesis steps described above are shown below. The deprotection, purification and coupling of compound 6.1a to prepare the compound 6.1b is further described below.

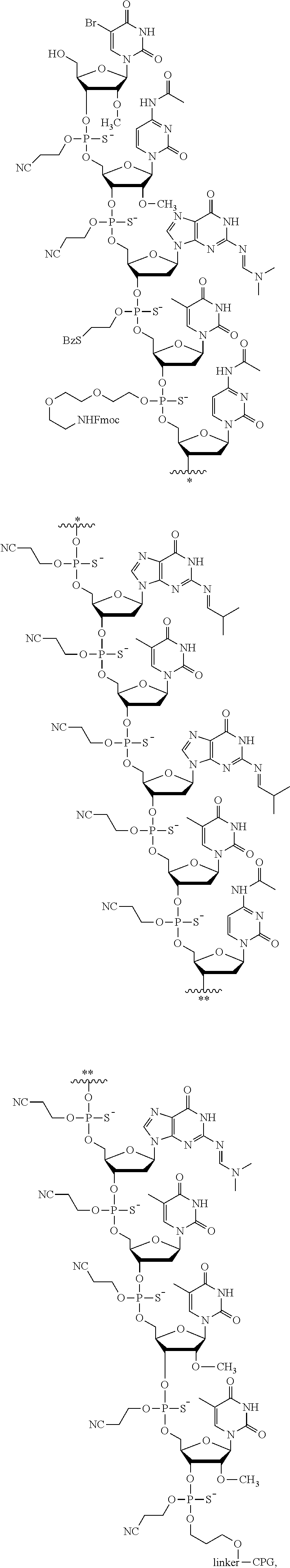

### Fmoc-Protected, CPG-Supported Compound 6.1a

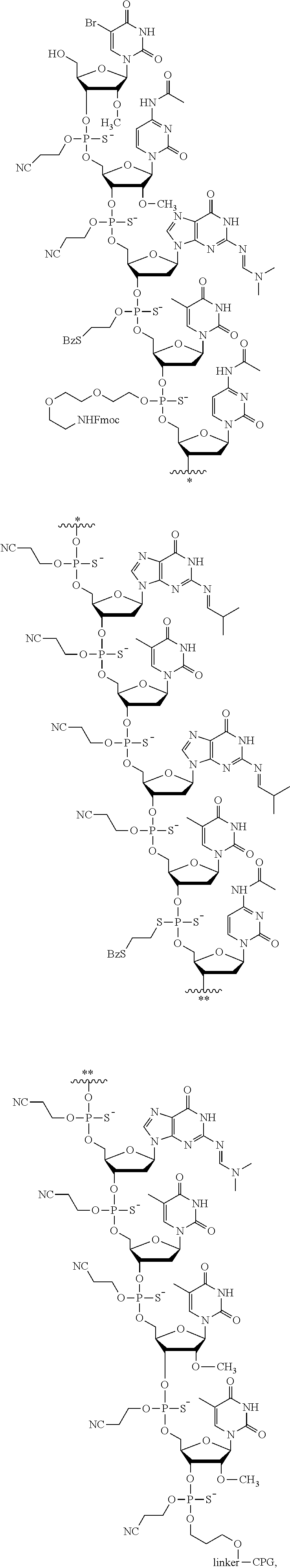

### Fmoc-Protected, CPG-Supported Compound 6.2a

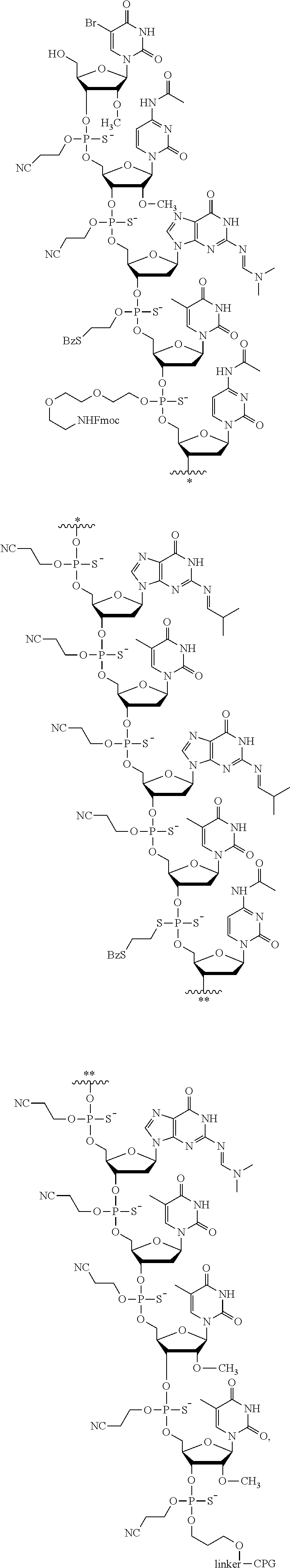

### Fmoc-Protected, CPG-Supported Compound 6.3a

The Fmoc-protected, CPG-supported oligonucleotide compound 6.1a obtained from the synthesis above was simultaneously cleaved from the support and deprotected by reacting the CPG support with 20 mM dithiothreitol in ammonium hydroxide:methylamine, 1:1 (v/v) for 2 hours at room temperature to give crude compound 6.1a. The crude product was purified by ion-pair reversed phase HPLC (IP—RP-HPLC) and its identity confirmed by ESI-MS. Crude compound 6.1a was purified by HPLC and desalted.

Compound 6.1a was subsequently reacted with O-[2-(Fmoc-amino)-ethyl]-0′-[3-(N-succinimidyloxy)-3-oxopropyl]polyethylene glycol (Fmoc-N-amido-dPEG₂₄-NHS ester) in sodium bicarbonate buffer to give Fmoc-protected compound 6.1b. Fmoc-protected compound 6.1b was reacted with 20 mM dithiothreitol in ammonium hydroxide:methylamine, 1:1 (v/v) for 2 hours at room temperature to give crude compound 6.1b. The crude product was purified by ion-pair reversed phase HPLC (IP—RP-HPLC) and its identity confirmed by ESI-MS. Crude compound 6.1b was purified by HPLC, desalted, and lyophilized to give the purified oligonucleotide 6.1b.

### Production of Antibodies

Antibodies generated in-house are typically expressed in suspension culture of Expi293 system (ThermoFisher) according to the manufacturer's manual. The expressed antibodies are purified via Protein A capture using MabSelectLX chromatography (GE), elution with 0.1M citrate (pH 3.3) and dialyzed in final buffer composition of 1×PBS (Phosphate Buffered Saline, pH 7.4). The afucosylated antibodies are typically produced with coexpression of compound 2-fluorofucose or 5-alkynylfucose derivatives in the Expi293 system.

### One-Step Conjugation Method Via mTG (Microbial Transglutaminase)

Q-tag with the sequence RPQGFGPP (SEQ ID NO: 49) was genetically linked to the C-terminus of the heavy chain of antibody. To perform conjugation, the purified antibody (containing the engineered Q tags at the C-terminal of heavy chain) were first buffer exchanged into 25 mM Tris, 150 mM NaCl pH 8. The Ab-Q-tag containing moiety and CpG were added in molar ratio of 1:1.3 and incubated overnight with a final concentration of 1% mTG (w/v) (Ajinomoto) at room temperature. Final concentration of antibody used for conjugation is generally ˜20-25 uM. Mixture was loaded to a Q Sepharose HP (GE) equilibrated in 20% Buffer B (40 mM Tris, 2M NaCl pH8) and 80% Buffer A (40 mM Tris, pH8). Column was washed with 5 column volumes of 20% Buffer B. Separation was achieved with using a linear gradient from 20% B to 60% B in 30 column volumes. DAR1 peak fractions (Q tag conjugated with one CpG moiety) were pooled and concentrated followed by a gel filtration step using S200 (GE). Monomeric peak fractions were pooled and concentrated.

### Biological Evaluation of CpG-Nucleotides and Antibody-CpG Nucleotide Conjugates

Trima residuals were received from Vitalant and diluted 1:4 with Phosphate Buffered Saline (PBS, Gibco). Diluted blood was split into two tubes and underplayed with 15 mL Ficoll-Paque (GE Healthcare). Tubes were centrifuged for 30 minutes at 400×g. PBMCs were collected from the interface and resuspended in FACS buffer (PBS with 0.5% Bovine Serum Albumin (Gibco)). B cells were purified by negative selection using the B Cell Isolation Kit II, human (Miltenyi Biotec) and LS columns (Miltenyi Biotec) according to manufacturer's protocol.

PBMCs were immediately plated onto a 96-well format (500K/well) in Complete RPMI (RPMI+10% FBS). Five-fold serial dilutions were added to the cells from 100 nM to 6.4 μM of antibody and conjugated antibody and 1 uM to 64 μM of CpG oligonucleotides at 37° C. under 5% CO2 for 48 to 96 hours. Cells were pelleted by centrifugation for five minutes at 400×g and stained at 4° C. in Fixable Viability Dye eFluor 780 (Thermo Fisher) diluted 1:4000 in PBS. Cells were centrifuged and stained at 4° C. in FACS buffer for 30 minutes containing FcR Blocking Reagent (Miltenyi Biotec), anti-CD19, anti-CD20, anti-CD40, anti-HLADR and anti-CD80 for B cell assays and anti-CD14, anti-CD3, anti-CD19, anti-CD14, anti-CD123, anti-CD 11c and anti-CD86 for pDC assays. Cells were centrifuged and washed twice in FACS buffer and fixed in 0.5% paraformaldehyde. CountBright™ Absolute Counting Beads (Thermo Fisher) were added to each well to count the number of cells. Cells were analyzed on Attune NxT Flow Cytometer (Thermo Fisher), with subsequent data analysis by Flowjo 10.7 (Treestar). Dead cells were excluded by gating on the eFluor 780-negative population. Lineage specific cells were first excluded (CD19, CD3, CD14) prior to gating CD123⁺CD11c⁻ cells to identify pDC and gating CD19+, CD20+ or CD19⁺CD20⁺cells to identify B cells.

### Example 1: Activities of Free Immunomodulating Oligonucleotides (CpGs) in Human PBMCs

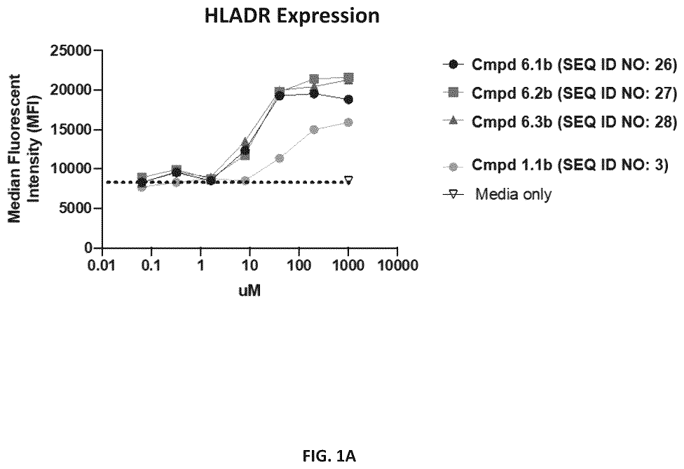

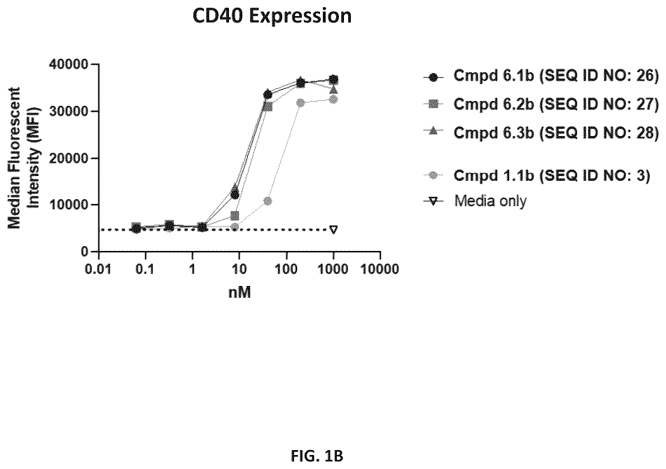

Human PBMCs were treated with free CpGs (SEQ ID NOs: 3 and 26-28) to evaluate their respective activities as observed by HLADR and CD40 expression on CD19 positive B cells (as shown in FIGS. 1A-1B). CpGs (SEQ ID NO: 26-28) all showed enhanced activities compared with CpG (SEQ ID NO: 3).

### Example 2: Activities of Immunomodulating Oligonucleotides

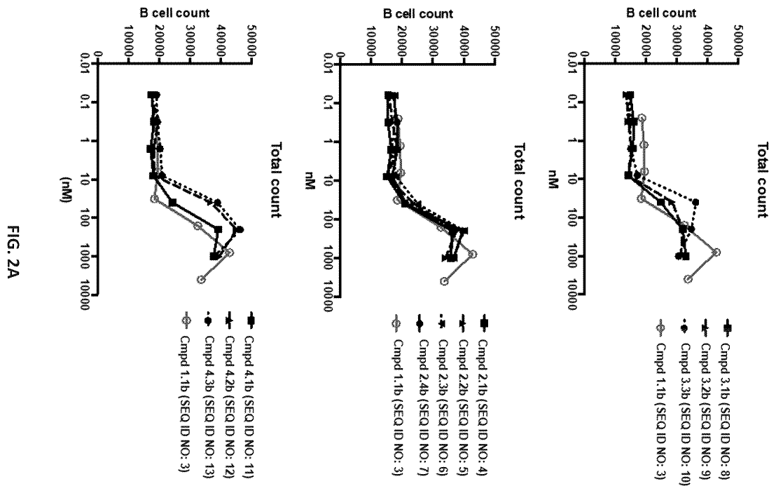

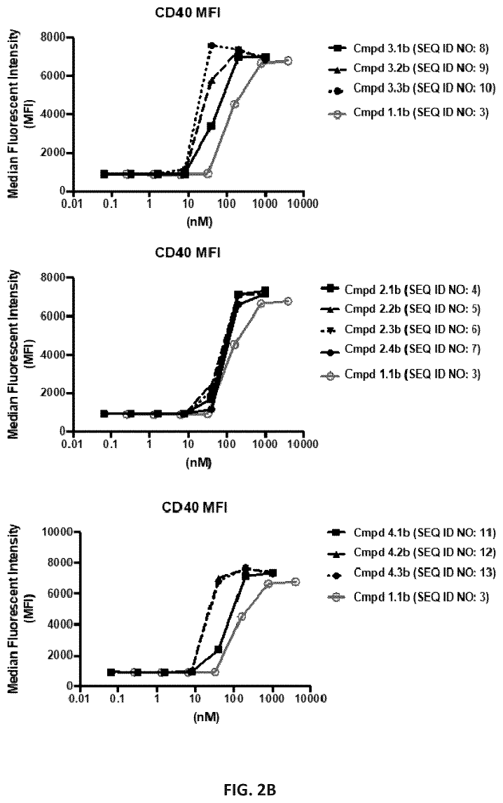

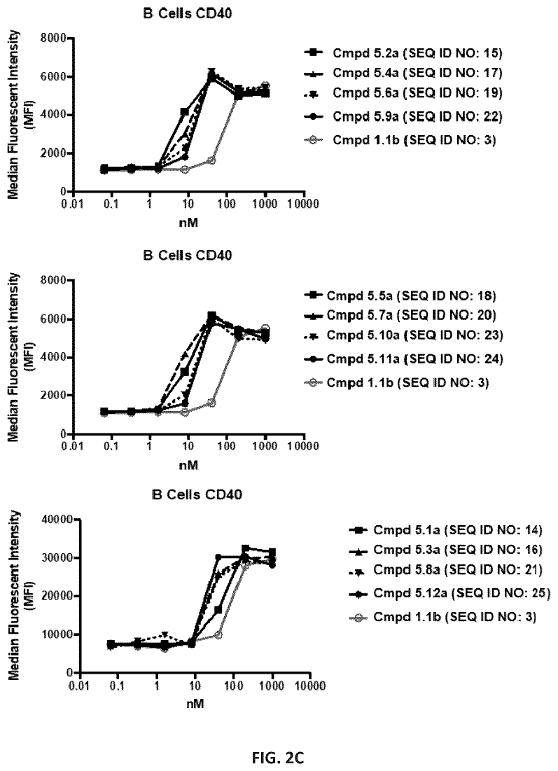

Various CpG oligonucleotides, SEQ ID NO: 3-25, were tested for their effects on proliferation and/or activation of B cells. FIGS. 2A-2C show the respective activities of select CpGs alone. All CpG oligonucleotides tested enhanced the activation of B cells after 48 hours of incubation. As determined by counting beads to calculate absolute B cell number and CD40 expression, all CpGs increased the number of B cells and CD40 expression. A select number of CpG oligonucleotides tested showed enhanced effects on B-cell proliferation and activation compared with CpG (SEQ ID NO: 3).

### Example 3: Transglutaminase-Mediated Conjugation

The transglutaminase-mediated conjugation was tested using an oligonucleotide A (with the sequence: tucgtcgtgacgtt, SEQ ID NO: 1) coordinated to a PEGylated linker (—NH—C(═O)—PEG₂₃-NH₂, structure shown below), and Q-tag peptides sequences SEQ ID NOs: 39-47 and 50-52.

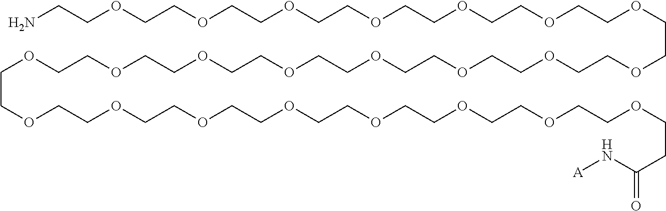

2 nmol of the Q-tag was added to 1 nmol of the linker in the present of 0.04 nmol of transglutaminase in PBS. The final concentration of linker is 50 μM. Reactions were kept at room temperature and quenched with 8 M formamide at 1 hour. The reaction solution was analyzed using reverse-phase HPLC with Xbridge C₁₈ column (4.6×150 mm) using solvent A (50 mM TEAA in water) and solvent B (Acetonitrile) with a gradient of 20% to 60% of solvent B in 10 minutes at 60° C. Alternatively, the reaction solution was analyzed using reverse-phase HPLC with Luna 3p C₁₈ column (4.6×50 mm) using solvent A (0.1% TFA in water) and solvent B (0.1% TFA in Acetonitrile) with a gradient of 10% to 70% of solvent B in 10 minutes at 50° C.

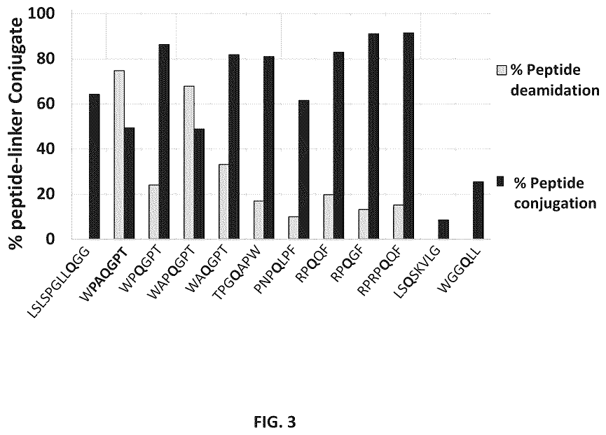

FIG. 3 shows the yields of the transglutaminase-mediated conjugation and peptide deamidation with various Q-tags. RPQGF (SEQ ID NO:47), RPQQF (SEQ ID NO:46), RPRPQQF (SEQ ID NO:50) showed high conjugate percentage and moderately low deamidation

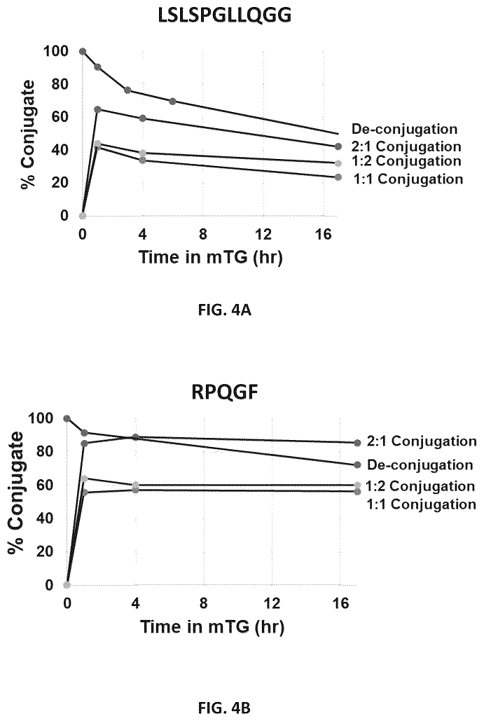

FIGS. 4A-4B show the conjugation and deconjugation of two conjugates prepared from Q-tag with SEQ ID NOs: 39 and 47 over time. RPQGF (SEQ ID NO:47) has higher percentage of conjugation with all Q-tag: linker+CpG ratio tested, over a duration of 16 hrs. Moreover, the deconjugation rate of RPQGF (SEQ ID NO:47) is also slower compared with LSLSPGLLQGG (SEQ ID NO:39).

### Example 4: Evaluation of Free CpG Activity on CD40 Expression by CD19+B Cells

### Materials and Methods

Trima residuals were received from Vitalant and diluted 1:2 with Phosphate Buffered Saline (PBS, Gibco). Diluted blood was split into two tubes and underplayed with 15 mL Ficoll-Paque (GE Healthcare). Tubes were centrifuged for 30 minutes at 400×g. PBMCs were collected from the interface, resuspended and washed in FACS buffer (PBS with 0.5% Bovine Serum Albumin (Gibco)). After one wash, PBMCs were resuspended in Complete RPMI (RPMI+10% FBS).

PBMCs were immediately plated onto a 96-well format (500K/well) in Complete RPMI. Five-fold serial dilutions were added to the cells from 1 uM to 64 μM of CpG oligonucleotides at 37° C. under 5% CO2 for 48 hours. Cells were pelleted by centrifugation for five minutes at 400×g and stained at 4° C. in Fixable Viability Dye eFluor 780 (Thermo Fisher) diluted 1:4000 in PBS. Cells were centrifuged and stained at 4° C. in FACS buffer for 30 minutes containing FcR Blocking Reagent (Miltenyi Biotec), anti-CD19, anti-CD20, anti-CD40, anti-HLADR and anti-CD80. Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (Treestar). Dead cells were excluded by gating on the eFluor 780-negative population. B cells were identified as CD19⁺CD20⁺cells and level of activation marker was assessed by median fluorescent intensity.

For Ramos NFkb Reporter Assay, Ramos-Blue Cells NF-kB/AP-1 Reporter B lymphocytes were purchased from Invivogen. Cells were grown and maintained in complete DMEM supplemented with 2 mM L-glutamine, 10% FBS, 100 ug/mL Normacin, Pen-Strep, 100 ug/mL Zeocin. Stimulation of the Ramos-Blue cells was performed. Briefly, cells were rinsed in growth medium without antibiotics. Cells were counted and resuspended in fresh complete DMEM without selection antibiotics at a density of 2×10⁶ cell/mL. 20 uL of 10 uM CpG 7-7 (7-7b; SEQ ID NO:35), CpG 12070 (SEQ ID NO:3) and ODN2006 titrated 1:5 were added to a flat-bottom 96-well plate, 180 uL of the cell suspension were added to a final concentration of 1 uM to 64 μM of CpG. Plate was incubated at 37° C. in a 5% CO2 incubator for 24 h. On day of assay, QB reagent and QB buffer were thawed before us. Quanti-Blue solution was prepared by adding 1 mL of QB reagent and 1 mL of 1 mL of QB buffer to 98 mL of sterile water in a sterile glass bottle. 180 uL of Quanti-Blue solution was dispensed per well into a new flat-bottom 96-well plate. 20 uL of supernatant from treated Ramos-Blue cells was then added to the 96-well plate. Plate was ten incubated for 6 h. Optical density was measured at OD655 using a plate reader (Molecular Devices), and data was tabulated in GraphPad Prism 9.0.

### Results

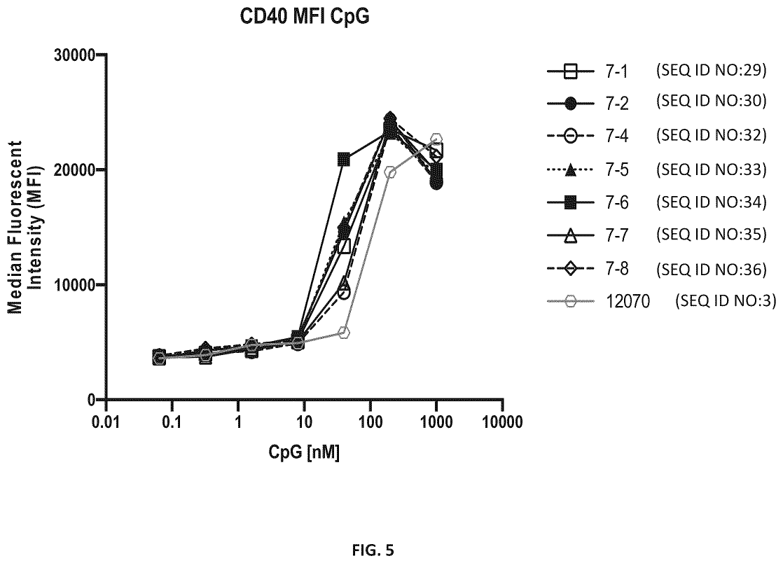

Human PBMCs were treated with free CpGs to evaluate their respective activities as observed by CD40 expression on CD19 positive B cells. As shown in FIG. 5, series 7 CpGs (SEQ ID NOS: 29, 30, and 32-36) all showed enhanced activities compared with CpG 12070 (SEQ ID NO: 3).

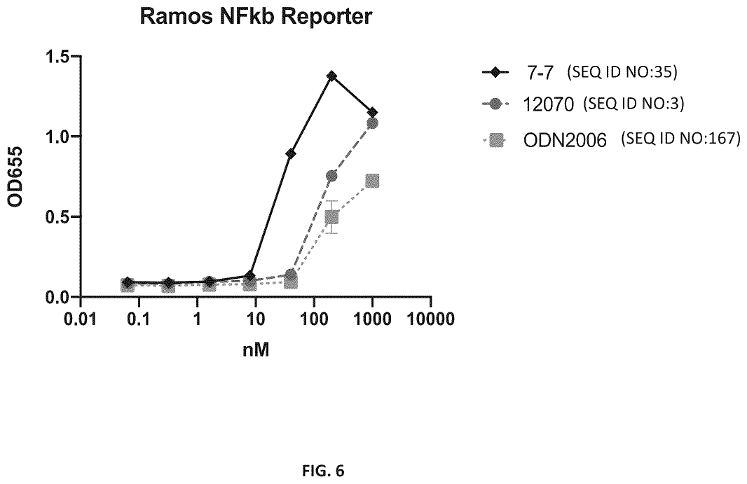

CpG oligos 7-7, 12070 and ODN2006 (5′-tcgtcgttttgtcgttttgtcgtt-3′; SEQ ID NO:167) were compared in a NFkb reporter assay. As shown in FIG. 6, CpG 7-7 showed significantly higher activity as compared to 12070 and ODN2006.

### Example 5: Evaluation of CpG Activity on PBMCs from Different Donors

The activity of CpG oligos 7-6, 7-7 and 12070 were compared for activity in PBMC cells from three different donor lines (D559, D804 and D643) as observed by CD40 expression. The evaluation of activity of the CpG oligos was performed using the same methods as Example 4 above.

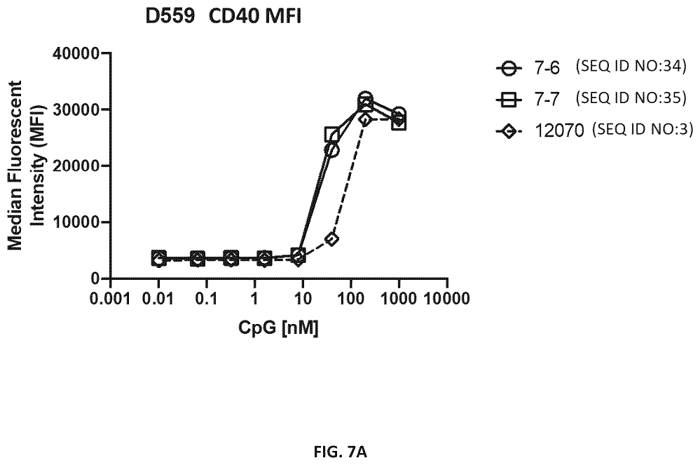

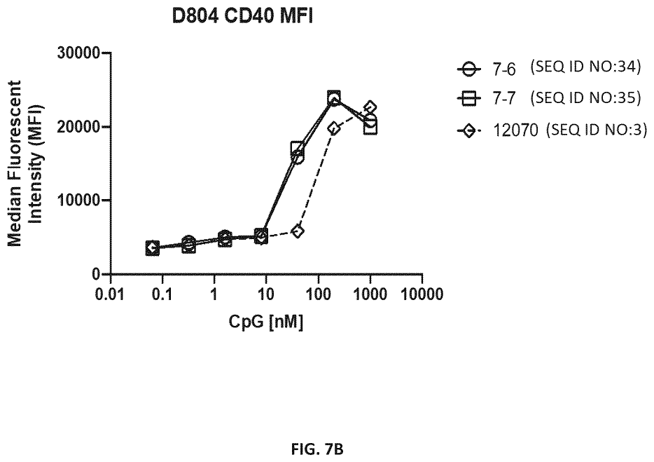

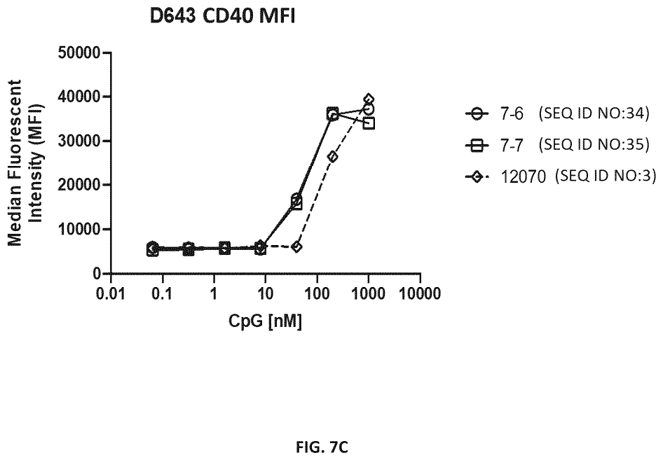

The results showed that the higher activities of 7-6 and 7-7 compared with 12070 were not dependent on the donor (FIGS. 7A-7C).

### Example 6: Contributions of 5′ Bromo 2′Deoxyuridine and PEG Linkage to CpG Activity

For evaluation of CpG oligonucleotides in human PBMCs, Trima residuals were received from Vitalant and diluted 1:4 with Phosphate Buffered Saline (PBS, Gibco). Diluted blood was split into two tubes and underplayed with 15 mL Ficoll-Paque (GE Healthcare). Tubes were centrifuged for 30 minutes at 400×g. PBMCs were collected from the interface and resuspended in FACS buffer (PBS with 0.5% Bovine Serum Albumin (Gibco)). PBMCs were immediately plated onto a 96-well format (500K/well) in Complete RPMI (RPMI+10% FBS). Five-fold serial dilutions were added to the cells from 1 uM to 64 μM of CpG oligonucleotides at 37° C. under 5% CO2 for 48 to 96 hours. Cells were pelleted by centrifugation for five minutes at 400×g and stained at 4° C. in Fixable Viability Dye eFluor 780 (Thermo Fisher) diluted 1:4000 in PBS. Cells were centrifuged and stained at 4° C. in FACS buffer for 30 minutes containing FcR. Blocking Reagent (Miltenyi Biotec), anti-CD19, anti-CD40, and anti-CD86. Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (Treestar). Dead cells were excluded by gating on the eFluor 780-negative population. Gating CD19+, CD20+ or CD19⁺CD20⁺cells to identify B cells. Data was tabulated using GraphPad Prism 9.0.

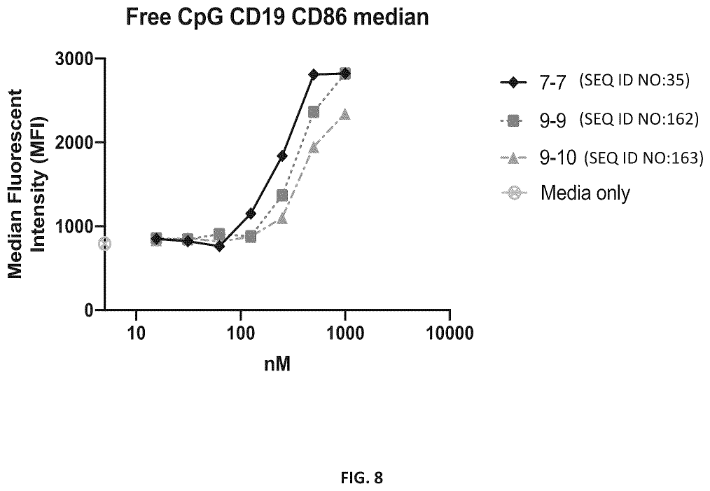

As shown in FIG. 8, CpG oligonucleotides 9-9 and 9-10 without the bromo modification at the 5′ uridine activated CD86 expression. This implies that the bromo modification is not an essential component of the respective oligonucleotides.

### Example 7: Generation of Antibodies Against Nectin-4

A proprietary human naïve B-cells phage library was used to screen for anti-human Nectin-4 antibodies. The methods used to generate the human naïve phage library was as described in Clackson et al., Nature 1991, 352, 624-628 or Marks et al., J. Mol. Biol., 1991, 222:581-597. For this human nectin-4 antibody discovery program, AcroBiosystems hNectin4-ECD-His-Avi protein (cat no: NE4-H₅₂H₃) and hNectin4 ECD-6His-Avi protein construct (TNT174; see Table 19) were used to complete 5 rounds of phage panning. The final enriched pooled phage clones were plated out for single clones. The amplified phage particles were screened by ELISA assay and the top clones were sequenced and analyzed.

IMMULON™ ELISA plates (THERMO SCIENTIFIC™; catalogue number 3855) were incubated overnight at room temperature with 50 uL/well of either 2 ug/mL human nectin-4 extracellular domain (ECD) (Acrobiosystems; catalogue number NE4-H₅₂H₃) in phosphate buffered saline pH 7.4 or 10% Bovine Serum Albumin (BSA) in PBS, washed with TBST (25 mM Tris, 0.15M NaCl, 0.05% Tween-20, pH 7.5) and blocked with 300 ul/well, 2% BSA in PBS, for 1 hour at room temperature. Plates were washed with TBST and 50 ul/well of freshly made phage clone cultures, diluted 1:100 in 2% BSA in PBS+0.01% Tween-20 and were added to wells coated with human Nectin-4 and to wells coated with 10% BSA and incubated for 1 hour at room temperature. Plates were washed with TBST before a 1 hour incubation of 100 ul/well, 0.5 ug/mL, anti-M13 phage coat protein g8p antibody diluted in 2% BSA in PBS+0.01% Tween-20. After washing with TBST, 100 ul/well of 0.5 ug/mL HRP conjugated goat anti-mouse IgG in 2% BSA in PBS+0.01% Tween-20 was added to the wells and incubated for 1 hour at room temperature. Wells were washed with TBST and 100 uL/well of 3,3′,5,5′-tetra-methylbenzidine peroxidase substrate was added to the plates for approximately 2-5 minutes. The reaction was terminated with 100 uL/well, 0.16M sulfuric acid and the resulting optical density (O.D.) absorbance was measured at 450 nm with a reference of 570 nm using a plate reader.

Clones were divided into four tiers based on O.D. of human nectin-4-coated wells divided by O.D. of BSA-coated wells. The tiers were >10-fold (38 out of 176 clones), >5-10-fold (43 out of 176 clones), 2-5-fold (62 out of 176 clones), and less than 2-fold (33 out of 176 clones).

Sequencing was carried out on the 38 clones with >10-fold titer. After sequence analyses, 11 clones were selected. Full-length recombinant monoclonal antibodies were generated using the chosen variable domains as mouse IgG2a antibodies. The antibodies were expressed in HEK293 suspension culture and purified via Protein A capture chromatography with 0.1M citrate (pH 3.3) elution and then dialyzed in final buffer composition of 1× PBS. The VL and VH sequences for the antibodies (individually termed TNT-188, TNT-189, TNT-190, TNT-191, TNT-192, TNT-193, TNT-194, TNT-195, TNT-196, TNT-197, and TNT-198 for the mouse IgG2a constructs) are summarized in Table 7 (VH domains) and Table 8 (VL domains). A reported nectin-4 antibody sequence is also generated and designated TNT-153 (VH and VL sequences also in Tables 7 and 8). The light chain and heavy chain sequences of the mouse IgG2a sequences are summarized in Tables 12 and 13, respectively.

The corresponding antibodies were also engineered as full-length antibodies with human IgG1. The human IgG1 antibodies were designated TNT-201, TNT-202, TNT-203, TNT-204, TNT-205, TNT-206, TNT-207, TNT-208, TNT-209, TNT-210, TNT-211, and TNT-212. The VH and VL domains are summarized in Table 7 and Table 8, respectively. The light chain and heavy chain sequences are summarized in Tables 14 and 15, respectively.

CDR identification according to Chothia, Kabat, and IMGT are summarized in Tables 9, 10, and 11, respectively, for each of these antibodies.

### Example 8: Characterization of Nectin-4 Antibodies

Initial testing of binding of nectin-4 antibodies to nectin-4. Binding of the nectin-4 antibodies described in the preceding example in mouse IgG2a to human nectin-4 ECD (TNT-175) was assessed by surface plasmon resonance (SPR) using Bio-Rad ProteOn™. Biotinylated protein A (15 μg/mL) was immobilized to the surface of an NLC chip. 30 nM of purified mAb supernatant was diluted in PBS-T and captured over the protein A surface. Serial dilutions of human nectin-4 were injected over the mAb-coated chips, and binding kinetics were determined. Chips were regenerated using 4:1 v/v of IgG elution butter/4M NaCl. Binding data are summarized in Table 18, below.

TABLE 18
Binding to human nectin-4

| Antibody | Ka (1/Ms) | Kd (1/s) | KD (M) |
|---|---|---|---|
| TNT-188 | 2.17E+05 | 1.53E−03 | 7.03E−09 |
| TNT-189 | 8.32E+05 | 4.23E−03 | 5.09E−09 |
| TNT-190 | 2.65E+05 | 8.31E−03 | 3.14E−08 |
| TNT-191 | 2.29E+05 | 9.83E−04 | 4.30E−09 |
| TNT-192 | 3.22E+05 | 2.94E−03 | 9.14E−09 |
| TNT-193 | 2.24E+06 | 8.37E−03 | 3.73E−09 |
| TNT-194 | 3.92E+06 | 2.30E−03 | 5.85E−10 |
| TNT-195 | 9.30E+05 | 1.25E−03 | 1.34E−09 |
| TNT-196 | 1.62E+06 | 2.45E−02 | 1.52E−08 |
| TNT-197 | 7.80E+05 | 3.34E−03 | 4.28E−09 |
| TNT-198 | 6.84E+04 | 1.38E−03 | 2.02E−08 |
| TNT-153 | 1.05E+06 | 3.99E−03 | 3.80E−09 |

All the nectin-4 antibodies bound to human nectin-4 in the range of 30 nM to 0.5 nM.

Further testing of select nectin-4 antibodies to human, mouse and cyno nectin-4. Additional assessment of the nectin-4 antibodies was performed to measure binding to human, mouse, and cynomolgus nectin-4. Biacore 8K (Cytiva, Marlborough, MA) was used to further characterize binding of four of the nectin-4 monoclonal antibodies. Their binding affinities to human nectin-4-ECD (TNT-175), mouse nectin-4-LCD (TNT-250), human nectin-4-IgV (TNT-252) domain construct, and a cyno-nectin4-ECD (TNT-255) protein construct were compared (see Table 19 for constructs).

TABLE 19
Protein constructs.

| Construct | Sequence |
|---|---|
| Human nectin-4 ECD- | GELETSDVVTVVLGQDAKLPCFYRGDSGEQVGQVAWARVDAGEGAQELALLH |
| 6His-Avi (TNT-174) | SKYGLHVSPAYEGRVEQPPPPRNPLDGSVLLRNAVQADEGEYECRVSTFPAGSF |
| | QARLRLRVLVPPLPSLNPGPALEE |
| | GQGLTLAASCTAEGSPAPSVTWDTEVKGTTSSRSFKHSRSAAVTSEFHLVPSRS |
| | MNGQPLTCVVSHPGLLQDQRITHILHVSFLAEASVRGLEDQNLWHIGREGAML |
| | KCLSEGQPPPSYNWTRLDGPLPS |
| | GVRVDGDTLGFPPLTTEHSGIYVCHVSNEFSSRDSQVTVDVLDPQEDSGKQVDL |
| | VSASHHHHHHGLNDIFEAQKIEWHE (SEQ ID NO: 488) |
| Human nectin-4 ECD-6His | GELETSDVVTVVLGQDAKLPCFYRGDSGEQVGQVAWARVDAGEGAQELALLH |
| (TNT-175) | SKYGLHVSPAYEGRVEQPPPPRNPLDGSVLLRNAVQADEGEYECRVSTFPAGSF |
| | QARLRLRVLVPPLPSLNPGPALEEGQGLTLAASCTAEGSPAPSVTWDTEVKGTT |
| | SSRSFKHSRSAAVTSEFHLVPSRSMNGQPLTCVVSHPGLLQDQRITHILHVSFLAE |
| | ASVRGLEDQNLWHIGREGAMLKCLSEGQPPPSYNWTRLDGPLPSGVRVDGDTL |
| | GFPPLTTEHSGIYVCHVSNEFSSRDSQVTVDVLDPQEDSGKQVDLVSASHHHHH |
| | H (SEQ ID NO: 489) |
| Mouse nectin-4 ECD-6His | GELETSDVVTVVLGQDAKLPCFYRGDPDEQVGQVAWARVDP |
| (TNT-250) | NEGIRELALLHSKYGLHVNPAYEDRVEQPPPPRDPLDGSVLLRNAVQADEGEYE |
| | CRVSTF |
| | PAGSFQARMRLRVLVPPLPSLNPGPPLEEGQGLTLAASCTAEGSPAPSVTWDTE |
| | VKGTQS |
| | SRSFTHPRSAAVTSEFHLVPSRSMNGQPLTCVVSHPGLLQDRRITHTLQVAFLAE |
| | ASVRG |
| | LEDQNLWQVGREGATLKCLSEGQPPPKYNWTRLDGPLPSGVRVKGDTLGFPPL |
| | TTEHSGV |
| | YVCHVSNELSSRDSQVTVEVLDPEDPGKQVDLVSASHHHHHH (SEQ ID |
| | NO: 490) |
| Mouse nectin-4 ECD-6his- | GELETSDVVTVVLGQDAKLPCFYRGDPDEQVGQVAWARVDP |
| Avi (TNT-176) | NEGIRELALLHSKYGLHVNPAYEDRVEQPPPPRDPLDGSVLLRNAVQADEGEYE |
| | CRVSTF |
| | PAGSFQARMRLRVLVPPLPSLNPGPPLEEGQGLTLAASCTAEGSPAPSVTWDTE |
| | VKGTQS |
| | SRSFTHPRSAAVTSEFHLVPSRSMNGQPLTCVVSHPGLLQDRRITHTLQVAFLAE |
| | ASVRG |
| | LEDQNLWQVGREGATLKCLSEGQPPPKYNWTRLDGPLPSGVRVKGDTLGFPPL |
| | TTEHSGV |
| | YVCHVSNELSSRDSQVTVEVLDPEDPGKQVDLVSASHHHHHH |
| | GLNDIFEAQKIEWHE (SEQ ID NO:501) |
| Human nectin-4-IgV-6His | GELETSDVVTVVLGQDAKLPCFYRGDSGEQVGQVAWARVDAGEGAQELALLH |
| (TNT-252) | SKYGLHVS |
| | PAYEGRVEQPPPPRNPLDGSVLLRNAVQADEGEYECRVSTFPAGSFQARLRLRV |
| | LVHHHH |
| | HH (SEQ ID NO: 491) |
| Human nectin-1 ECD-6His | QVVQVNDSMYGFIGTDVVLHCSFANPLPSVKITQVTWQKSTNGSKQNVAIYNPS |
| (TNT-260) | MGVSVL |
| | APYRERVEFLRPSFTDGTIRLSRLELEDEGVYICEFATFPTGNRESQLNLTVMAKP |
| | TNWI |
| | EGTQAVLRAKKGQDDKVLVATCTSANGKPPSVVSWETRLKGEAEYQEIRNPNG |
| | TVTVISR |
| | YRLVPSREAHQQSLACIVNYHMDRFKESLTLNVQYEPEVTIEGFDGNWYLQRM |
| | DVKLTCK |
| | ADANPPATEYHWTTLNGSLPKGVEAQNRTLFFKGPINYSLAGTYICEATNPIGTR |
| | SGQVE |
| | VNITEFPYTPSPPEHGRRAGPVPTAIIGHHHHHH (SEQ ID NO: 492) |
| Cyno nectin-4 ECD-6His | GELETSDVVTVVLGQDAKLPCFYRGDSGEQVGQVAWARADAGEGAQELALLH |
| (TNT-255) | SKYGLHVS |
| | PAYEGRVEQPPPPRNPLDGSVLLRNAVQADEGEYECRVSTFPAGSFQARLRLRV |
| | LVPPLP |
| | SLNPGPALEEGQGLTLAASCTAEGSPAPSVTWDTEVKGTTSSRSFKHSRSAAVTS |
| | EFHLV |
| | PSRSMNGQPLTCVVSHPGLLQDQRITHILHVSFLAEASVRGLEDQNLWHVGREG |
| | AMLKCL |
| | SEGQPPPSYNWTRLDGPLPSGVRVDGDTLGFPPLTTEHSGIYVCHVSNEFSSRDS |
| | QVTVD |
| | VLDPQEDSGKQVDLVSASHHHHHH (SEQ ID NO: 493) |

All experiments were performed at 25° C. temperature and in HBS-EP+ run buffer (10 mM HEPES, pH 7.4, 150 mM NaCl, 3 mM EDTA, 0.05% Tween-20). The antibodies were captured or immobilized on a Biacore chip either through amine-coupling method on a CM5 or captured by ProteinA chip or captured by anti-mouse polyclonal antibody or anti-human polyclonal antibody. Either single cycle kinetics or multicycle kinetics was performed using a 3× or 5× serial dilution of antigen proteins from 1 nM up to 900 nM, depending on affinity. The data were processed and analyzed in Biacore Insight Evaluation Software using double-reference subtraction of the data and then fitted to a simple Langmuir binding model using global kinetic rate constants for association (Kₐ), dissociation (K_{d}), and Rₘₐₓ values per sample. Fit quality was determined by inspection of the residuals and of statistical T values and the equilibrium dissociation (or affinity) constant was deduced by the ratio of the kinetic rate constants. K_{D}=K_{d}/Kₐ.

Compared to TNT-212, a reported nectin-4 antibody. TNT-207 and TNT-208 bind with higher affinity to human nectin-4 ECD, human nectin-4-IgV and cyno nectin-4 ECD. TNT-210 has comparable binding affinities as TNT-212 to both human nectin-4 ECD and cyno human nectin-4 ECD. TNT-203. TNT-207, TNT-208 and TNT-210 also bound mouse nectin-4 ECD with varied affinity from 115 nM to 6 nM. High quality kinetics data of TNT-212 binding to mouse nectin-4 ECD were not obtained in current assay format. The binding results are summarizing in Table 20, below.

TABLE 20
Binding of antibodies to nectin-4 proteins

| | Human | Mouse | Human | Cyno |
|---|---|---|---|---|
| | nectin-4 | nectin-4 | nectin- | nectin-4- |
| | ECD | ECD | 4-IgV | ECD |
| Antibody | (K_{D}; nM) | (K_{D}; nM) | (K_{D}; nM) | (K_{D}; nM) |
| TNT-203 | 70 | 115 | 138 | 172 |
| TNT-207 | 1.5 | 6 | 1.8 | 4.2 |
| TNT-208 | 5 | 21 | 5.4 | 12 |
| TNT-210 | 12 | 12 | 17 | 36 |
| TNT-212 | 8 | — | 10 | 40 |

Four nectin-4 antibodies from Table 20 (TNT-203, TNT-207, TNT-208, and TNT-210) were conjugated to CpG 7-7 (7-7b; SEQ ID NO:35) and the conjugates bound human nectin-4 ECD antigen with the same K_{D} as unconjugated antibodies (data not shown). This indicates conjugation of CpG oligonucleotides to nectin-4 antibodies do not perturb binding of the nectin-4 antibodies to nectin-4.

Cell binding of nectin-4 antibodies. Cell binding of the nectin-4 antibodies was also assessed. MNK74 (nectin-4 positive) and AGS (nectin-4 negative) cells were obtained from ATCC and cultured according to their protocols. Cells were detached with TrypLE (Gibco) and washed by centrifugation for five minutes at 400×g with FACS buffer (PBS+2% FBS). Cells were resuspended in FACS buffer, plated in 96-well format (0.25 e6/well) then pelleted by centrifugation for five minutes at 400×g. Cells were stained at 4° C. in Fixable Viability Dye eFluor 506 (eBioscience) diluted 1:4,000 in PBS. Cells were centrifuged and stained at 4° C. in FACS buffer for 30 minutes containing FcR Blocking Reagent (Miltenyi Biotec), and with each one of antibodies (TNT-188, TNT-189, TNT-190, TNT-191, TNT-192, TNT-193, TNT-194, TNT-195, TNT-196, TNT-197, TNT-198, or TNT-153). Cells were centrifuged, washed once in FACS buffer and stained at 4° C. with Goat F(ab′)₂ anti-Mouse IgG APC (Southern Biotech) diluted 1:400 in FACS buffer for 30 minutes or appropriate anti-human IgG. Cells were centrifuged, washed twice and fixed in 0.5% paraformaldehyde. Cells were analyzed on Attune NxT Flow Cytometer (Thermo Fisher), with subsequent data analysis by Flowjo 10.7 (BD). Dead cells were excluded by gating on the eFluor 506-negative population. Nectin4 expression levels were confirmed via positive control mouse anti-human nectin-4-AF488 (R&D Systems). Binding was assessed by median fluorescent intensity.

All nectin-4 antibodies tested (TNT-188 to TNT-198 and TNT-153) bound MNK74 cells expressing human nectin-4 specifically and did not bind AGS cells, which do not express human nectin-4. The results are summarized in the table, below.

TABLE 21
Binding of antibodies to nectin-4-positive cells

| | Fold-change of binding to MNK74 |
|---|---|
| Antibody | compared to control AGS |
| TNT-188 | 4.3 |
| TNT-189 | 5 |
| TNT-190 | 5.1 |
| TNT-191 | 5 |
| TNT-192 | 4.5 |
| TNT-193 | 5.2 |
| TNT-194 | 7 |
| TNT-195 | 6.4 |
| TNT-196 | 6 |
| TNT-197 | 4.8 |
| TNT-198 | 5.9 |
| TNT-153 | 7.4 |

Competition experiments of nectin-4 antibodies. ELISA plates were coated overnight at 4° C. with goat anti-mouse IgG at 1 mg/mL in PBS. Plates were washed 3× with 300 uL ELISA wash buffer (25 mM Tris, 150 mM NaCl, 0.05% Tween20) then incubated with 1 mg/mL capture antibody (TNT-153 and TNT-188 to TNT-198, nectin-4 antibodies as mouse IgG2a) for one hour at room temperature. Concurrently, a premix of each antibody (TNT-201-TNT212, nectin-4 antibodies as human IgG1) at 2 mg/mL was incubated for one hour at room temperature in diluent (PBS with 0.5% BSA) with nectin-4-ECD-6His-Avi (TNT-175) dilutions from 2:1 molar ratio of TNT-175 to each antibody down to 0.031:1 molar ratio. As positive control, nectin-4 was diluted at same concentrations without a premix antibody. Two wells per plate were allocated for diluent only as negative/background control. Plates were washed as previously, 50 μL/well of premix was added to each plate and incubated for one hour. Plates were washed then incubated at room temperature for one hour with Rabbit anti 6× HIS HRP diluted 1:4,000 in diluent. Plates were washed once more and incubated at room temperature for 20 minutes with 50 μL/well of ABTS substrate. Plates were read at 405 nm, data were exported and analyzed via Excel. Binding was measured as an Optical Density (O.D.) value normalized to background and calculated as a percentage of positive control at the lowest nectin-4 dilution for greatest resolution. 100% indicates the ability to bind the antibody-nectin-4 complex indicating a non-blocking antibody, whereas 0% indicates no binding was observed, indicating a blocking antibody.

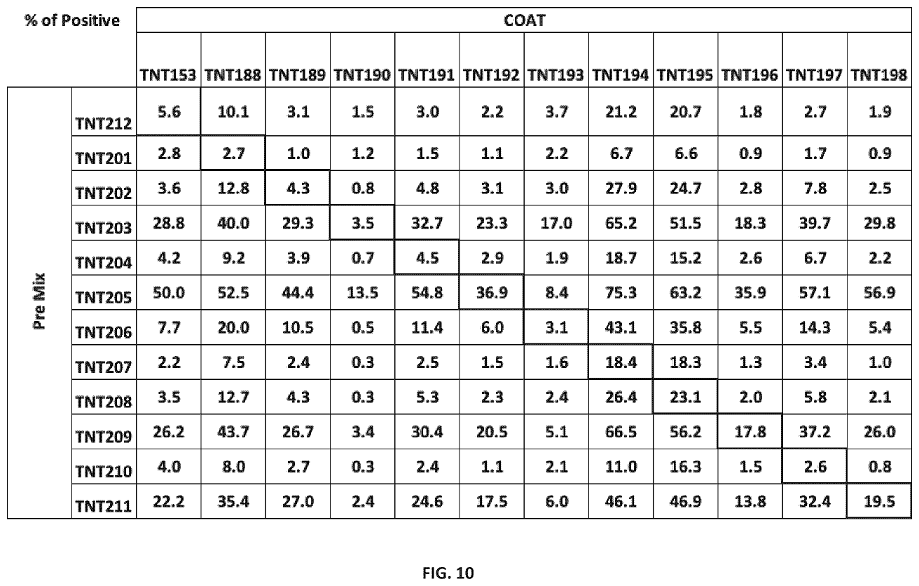

All antibodies inhibited each other's ability to bind human nectin-4. The extent of blocking correlated well with off rates (K_{d} 1/s) and overall affinity of each antibody. Those with faster off rates such as TNT-205 had difficulty fully blocking themselves. In a premix format, these antibodies can be kicked off by higher affinity capture antibodies. When assessing binding in both capture and premix orientations, none of the nectin-4 antibodies are able to sandwich and therefore all can be characterized as blockers of one another. The results are summarized in FIG. 10.

### Example 9: Internalization of Nectin-4 Antibodies

Antibodies TNT201-TNT212 were labelled with AlexaFluor-647 using Protein Labeling Kit (Invitrogen) according to manufacturers' protocol. In brief, 100-150 μg of an antibody in the sodium bicarbonate buffer (pH8.3), were incubated with reactive dye reagents under gentle agitation at room temperature for 1 hour. Unincorporated dyes were removed by size exclusion chromatography using purification resins and columns provided in the kits. Human nectin-4 expressing cells, T47D were sourced from ATCC and cultured according to their recommendations. Cells were detached with TrypLE (Gibco) and washed by centrifugation for five minutes at 400×g with FACS buffer (PBS with 1% BSA). Cells were resuspended in FACS buffer and aliquoted in 11 tubes, one for each sample at 1 mL each for a total of 2 million cells per tube. Each tube was stained with 10 g labeled TNT antibody for 60 minutes at 4° C. Cells were washed twice with FACS buffer and resuspended in 1 mL FACS buffer per sample. Cells were kept on ice in dark until a 100 uL aliquot per sample tube was shifted to 37° C. under 5% CO2 incubator at time −3 hours, −2 hours, −1 hour, −45 minutes, −30 minutes, −15 minutes, −10 minutes, −5 minutes for internalization such that all timepoints are complete at the same time. Upon removal from incubator, cells were washed with FACS buffer then resuspended in 100 μL Papain (Papain from papaya latex, Sigma) at 23.9 U/mL diluted in PBS (Gibco) and incubated for 30 minutes at 37° C. Cells were then washed and stained for 30 minutes at 4° C. in Fixable Viability Dye eFluor 506 (eBioscience) diluted 1:2000 in PBS (Gibco). Cells were washed once more with FACS buffer and fixed in 0.5% paraformaldehyde. Cells were analyzed on Attune NxT Flow Cytometer (Thermo Fisher), with subsequent data analysis by Flowjo 10.7 (BD). Dead cells were excluded by gating on the eFluor 506-negative population. Samples were assessed by median fluorescent intensity for each timepoint. The 4° C. sample at 0-minute timepoint was used as a “no internalization” control and thus considered to have 100% antibody-AlexaFluor-647 localized on cell surface. Therefore, internalized antibody-AlexaFluor-647 was calculated as the difference between the surface-localized antibody at 4° C. and that at 37° C. and expressed as a percentage relative to the value of the 4° C. sample.

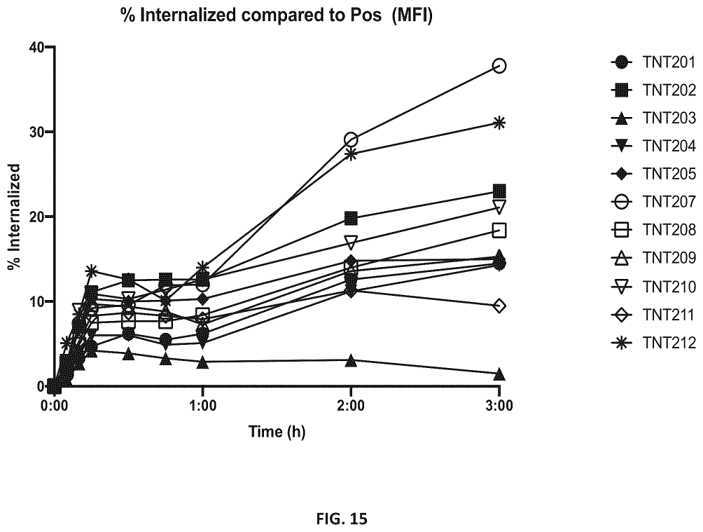

All TNT antibodies exhibited internalization except TNT203, which showed little to no internalization. Highest internalization levels were seen with TNT207, TNT210, TNT208 and TNT202. TNT207 appears to exhibit higher internalization rates than TNT212. Results are summarized in Table 22, below, and FIG. 15.

TABLE 22
Nectin-4 antibody internalization

| Antibody | % internalized (at 3 hours) |
|---|---|
| TNT-201 | 14.5% |
| TNT-202 | 23.0% |
| TNT-203 | 1.5% |
| TNT-204 | 14.3% |
| TNT-205 | 15.0% |
| TNT-207 | 37.8% |
| TNT-208 | 18.4% |
| TNT-209 | 15.3% |
| TNT-210 | 21.1% |
| TNT-211 | 9.5% |
| TNT-212 | 31.1% |

### Example 10: Conjugation of Nectin-4 Antibodies to CpG Oligonucleotide

A conjugation tag with the sequence RPQGFGPP (SEQ ID NO:49) was engineered onto the C-terminal of all the recombinant monoclonal antibody heavy chain expression constructs. This enabled conjugation of the CpG oligonucleotide to the C-terminus of the antibody. All DNA constructs were codon optimized and synthesized by ATUM (Menlo Park, CA, USA). All antibodies were transiently expressed in Expi293 expression system (Life Technology, USA). For conjugation with CpG oligonucleotides, the Protein A affinity chromatography purified nectin-4 antibody was added to the conjugation reaction at 5-20 uM, incubated with 1-10 ug of microbial transglutaminase with appropriate molar ratio of CpG oligonucleotides to mAb for overnight at room temperature. The appropriate DAR peak fractions from anion exchange chromatography are further purified by size-exclusion chromatography or other polishing methods if needed. The final antibodies and their CpG conjugates are in 1×PBS pH 7.4 for in vitro and in vivo assays. Each of antibodies TNT-201 to TNT-212 in human IgG1 with Q-tag (i.e., the light chain and heavy chains disclosed in Tables 14 and 15, respectively) were prepared and conjugated to the respective CpG oligonucleotides. The list of nectin-4 conjugates and their designations are shown in Table 23. TNT-347xx is TNT-337 antibody conjugated to TLR8 agonist. TNT-381xx is TNT-212 antibody conjugated to MMAE.

TABLE 23
List of nectin-4 conjugates.

| Conjugate designation | Antibody used for the conjugation |
|---|---|
| TNT-169a | TNT-154* |
| TNT-354a | TNT-201* |
| TNT-264a | TNT-203* |
| TNT-364a | TNT-204* |
| TNT-265a | TNT-207* |
| TNT-266a | TNT-208* |
| TNT-267a | TNT-210* |
| TNT-357a | TNT-211* |
| TNT-358a/TNT-169a | TNT-212* |
| TNT-359a | TNT-317* |
| TNT-360a | TNT-321* |
| TNT-361a | TNT-323* |
| TNT-365a | TNT-324* |
| TNT-349a | TNT-327* |
| TNT-350a | TNT-328* |
| TNT-362a | TNT-332* |
| TNT-351a | TNT-333* |
| TNT-352a | TNT-335* |
| TNT-363a | TNT-208 non-fucosylated* |
| TNT-348a | TNT-208** |
| TNT-373a | TNT-327** |
| TNT-374a | TNT-328* |
| TNT-375a | TNT-333** |
| TNT-302a | TNT-194** |
| TNT-303a | TNT-195** |
| TNT-304a | TNT-197** |
| TNT-273a | TNT-153** |
| TNT-372a | TNT-201* |
| TNT-377b | TNT-201*** |
| TNT-385a | TNT-201* non-fucosylated |
| TNT-415a | TNT-412* |
| TNT-416a | TNT-414* |

*conjugation is to CpG 7-7 (SEQ ID NO: 35) as DAR1
**conjugation is to CpG 4523 (SEQ ID NO: 499) as DAR1
***conjugation is to CpG 4523 (SEQ ID NO: 499) as DAR2

### Example 11: Monocyte Activation by Nectin-4 Antibody-CpG Conjugates

Trima residual was received from Vitalant and diluted 1:2 with PBS. Diluted blood was underlaid with 15 mL Ficoll-Paque. Tubes were centrifuged for 30 minutes at 400×g. PBMCs were collected from the interface, resuspended and washed in FACS buffer (PBS with 0.5% BSA). After one wash, PBMCs were resuspended in Complete RPMJ1640 (RPMJ1640+10% FBS). PBMCs were immediately plated onto a 96-well format (0.5 e6/well) in Complete RPML. Tumor cells (0.025E6/well) from DLD-1 parental labeled with CellTrace CFSE (ThermoFisher) or DLD-1 transduced to overexpress nectin-4 and GFP were added to PBMC at 20:1 (effector to target). Three-fold serial dilutions from 300 nM to 0.41 nM of nectin-4 antibody-CpG conjugates were added to the cells at 37° C. under 5% CO₂ for 18 hours. Cells were pelleted by centrifugation for five minutes at 400×g and stained at 4° C. in Fixable Viability Dye eFluor 780 (ThermoFisher) diluted 1:4000 in PBS. Cells were centrifuged and stained at 4° C. in FACS buffer for 30 minutes containing FcR Blocking Reagent (Miltenyi Biotec), anti-CD326, anti-CD14, anti-CD11c, anti-CD3, anti-CD19, anti-CD56, anti-CD16, anti-HLADR, anti-CD40, anti-CD86 (ThermoFisher, Biolegend). Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (BD). Dead cells were excluded by gating on the eFluor 780-negative population. Monocytes were identified as CD3-CD19-CD14⁺cells. CD14⁺monocyte activation levels were assessed by median fluorescent intensity.

As shown in FIG. 11, there was robust induction of CD86 on human monocytes upon nectin-4-CpG conjugates engagement of nectin-4-positive tumor cells and human monocytes. The antibody conjugates tested were TNT264a, TNT265a, TNT266a, TNT267a, and TNT169a. In general, all of the CpG conjugates showed higher activity when tested in the nectin-4-positive tumor cells versus that of nectin-4 negative parental DLD-1 tumor cells.

### Example 12: Dendritic Cell Activation by Nectin-4 Antibody-CpG Conjugates

Trima residual was received from Vitalant and diluted 1:2 with PBS. Diluted blood was underlaid with 15 mL Ficoll-Paque (GE Healthcare). Tubes were centrifuged for 30 minutes at 400×g. PBMCs were collected from the interface, resuspended and washed in FACS buffer (PBS with 0.5% BSA). After one wash, PBMCs were resuspended in Complete RPMI1640 (RPMI1640+10% FBS). PBMCs were immediately plated onto a 96-well format (0.5 e6/well) in Complete RPMI. Tumor cells (0.025E6/well) from DLD-1 parental labeled with CellTrace CFSE (ThermoFisher), or DLD-1 transduced to overexpress nectin-4 and GFP were added to PBMC at 20:1 (effector to target). Three-fold serial dilutions were added to the cells from 300 nM to 0.41 nM of nectin-4-CpG conjugates at 37° C. under 5% CO2 for 18 hours. Cells were pelleted by centrifugation for five minutes at 400×g and stained at 4° C. in Fixable Viability Dye eFluor 780 (Thermo Fisher) diluted 1:4000 in PBS. Cells were centrifuged and stained at 4° C. in FACS buffer for 30 minutes containing FcR Blocking Reagent (Miltenyi Biotec), anti-CD326, anti-CD14, anti-CD11c, anti-CD3, anti-CD19, anti-CD56, anti-CD16, anti-HLADR, anti-CD40, anti-CD86 (ThermoFisher, Biolegend). Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (BD). Dead cells were excluded by gating on the eFluor 780-negative population. DCs were identified as CD3-CD19-CD14-CD56-HLADR⁺CD11c⁺ cells. DC activation levels were assessed by median fluorescent intensity.

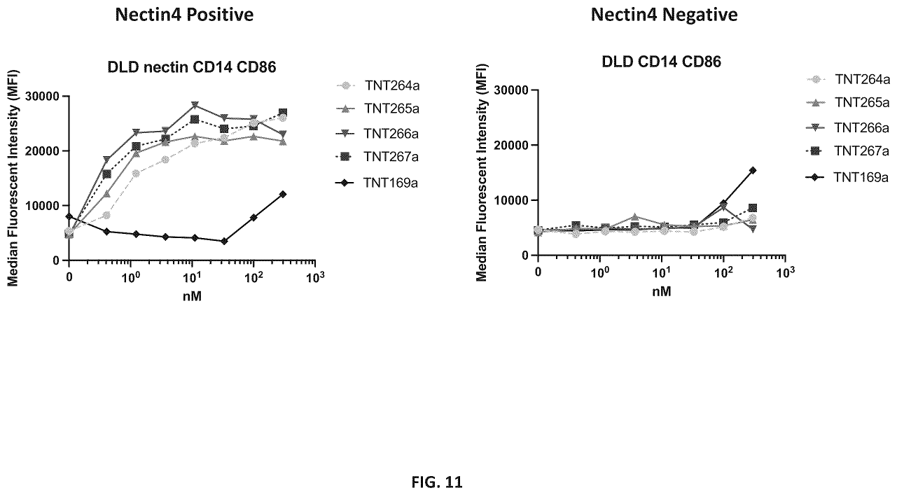

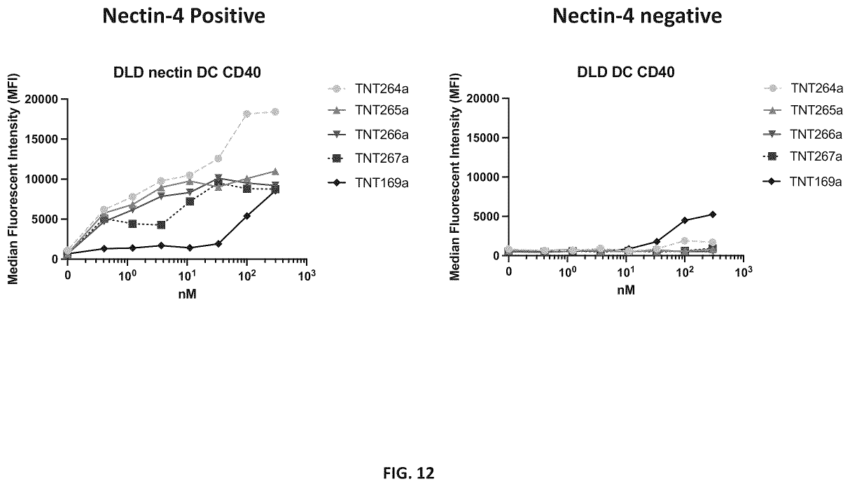

As shown in FIG. 12, the nectin-4 CpG conjugates engagement of nectin-4 positive tumor cells and human DCs showed robust induction of CD40. The antibody conjugates tested were TNT264a, TNT265a, TNT266a, TNT267a, and TNT169a. In contrast, limited to no induction of CD40 was observed in the context of nectin-4 negative tumor cells.

### Example 13: Enhancement of Tumor Phagocytosis

Human CD14⁺cells were purified from Trima residuals (Vitalant) with Ficoll-Paque Plus (GE) and negative selection (Monocyte Isolation Kit II, Miltenyi Biotec) according to the manufacturers' protocols. Monocyte-derived macrophages polarized to M2 phenotype were made by seeding 10 million CD14⁺ cells into 150 mm tissue culture dishes (Corning) in RPMI1640 supplemented with 10% FBS and 50 ng/mL MCSF (Miltenyi Biotec). Cells were cultured for 7-11 days. DLD-1 target cells were transduced to overexpress nectin-4 and GFP. Adherent cells were detached from culture plates with TrypLE Select (Thermo Fisher). 100,000 target cells and 50,000 macrophages were incubated in ultra-low attachment U-bottom 96-well plates (Corning) with three-fold serial dilutions from 1 uM to 1.37 nM of CpG oligonucleotide or 300 nM to 0.41 nM of nectin-4 antibody conjugates at 37° C. under 5% CO2 for 2 hours. For flow cytometry, cells were incubated in human FcR blocking reagent (Miltenyi Biotec) and stained with fluorochrome-labeled antibodies against CD33 and CD14 (ThermoFisher). To eliminate macrophage/target cell adhesion from analyses, antibody against CD326 (ThermoFisher) was included. Furthermore, a pulse geometry gate of forward scatter signal area vs height was used to select for single cells. Fixable viability dye eFluor 780 (Thermo Fisher) was used to identify live cells. Cells were acquired on a FACS Canto II flow cytometer (BD Biosciences) with subsequent analysis using FlowJo 10.7 software (BD). Percent phagocytosis indicates the percentage of viable CD33⁺CD14⁺macrophages that stain negative for CD326 and positive for CFSE.

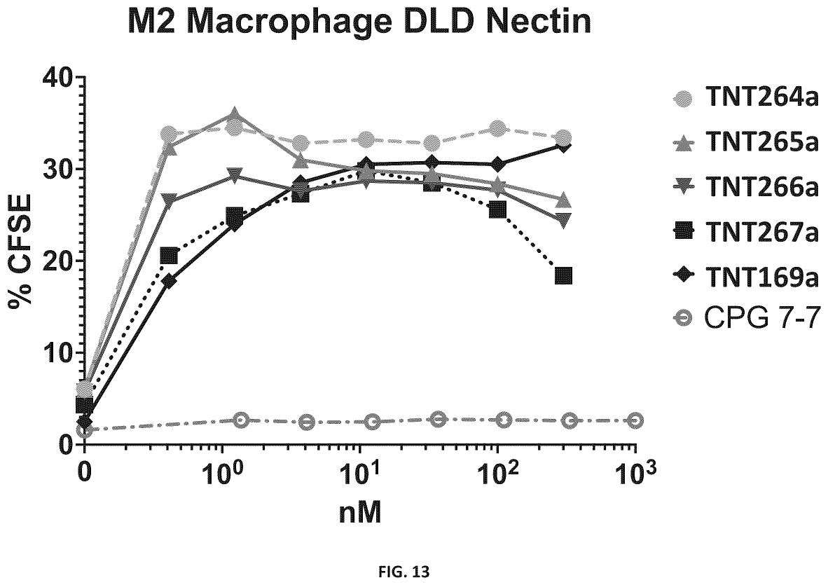

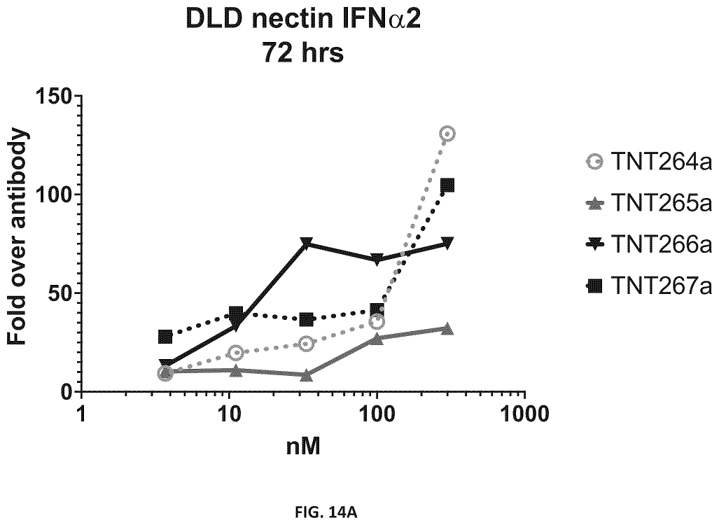

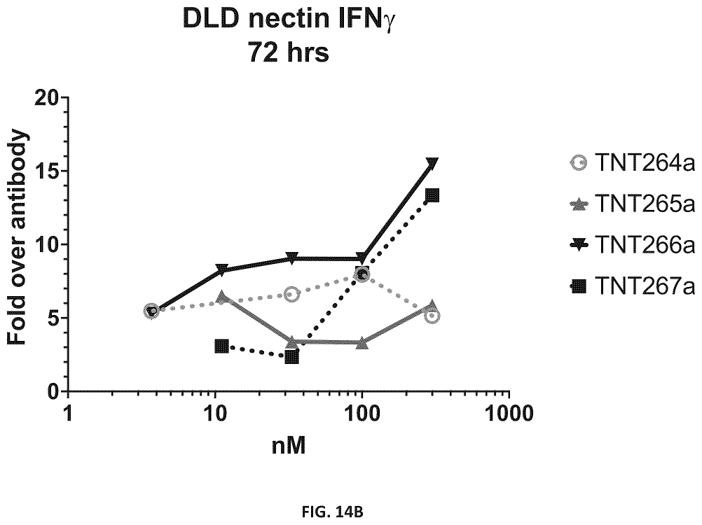

As shown in FIG. 13, there was robust phagocytosis of DLD-1 nectin-4 tumor cells by macrophages with nectin-4 CpG conjugates. The conjugates tested were TNT264a, TNT265a, TNT266a, TNT267a, TNT169a. All conjugates showed substantially higher activity whereas the unconjugated CpG oligonucleotide (CpG 7-7) did not appear to result in detectable phagocytosis.

### Example 14: Enhancement of Cytokine Secretion

Trima residuals were received from Vitalant and diluted 1:2 with PBS. Diluted blood was underlaid with 15 mL Ficoll -Paque (GE Healthcare). Tubes were centrifuged for 30 minutes at 400×g. PBMCs were collected from the interface, resuspended and washed in FACS buffer (PBS with 0.5% Bovine Serum Albumin, Gibco). After one wash, PBMCs were resuspended in Complete RPMI1640 (RPMI1640+10% FBS, Gibco). PBMCs were immediately plated onto a 96-well format (1 e6/well) in Complete RPMI1640. Tumor cells (0.025E6/well) from DLD-1 transduced to overexpress nectin-4 and GFP were added to PBMC at 20:1 (effector to target). Three-fold serial dilutions from 300 nM to 0.41 nM of nectin-4 antibody conjugates were added to the cells from at 37° C. under 5% CO2 for 72 hours. Cells were pelleted by centrifugation for five minutes at 400×g and supernatant was collected for cytokine quantified using BioLegend LEGENDplex™ Human Inflammation Panel 1 bead-based immunoassay per manufacturer's recommendation. Briefly, supernatant plated in assay buffer prior to addition of cytokine specific capture beads. Plate was incubated for 2 hours at room temperature followed by two washes prior to the addition of biotinylated detection antibody for 1 hour. Without washing, PE conjugated streptavidin was added to the plate and incubate for 30 minutes at room temperature followed by two washes prior to the addition of buffer. Assay plate was then processed by flow cytometry using Attune NxT cytometer (Thermofisher). Data were analyzed with FlowJo 10.7 software (BD) and tabulated using GraphPad Prism.

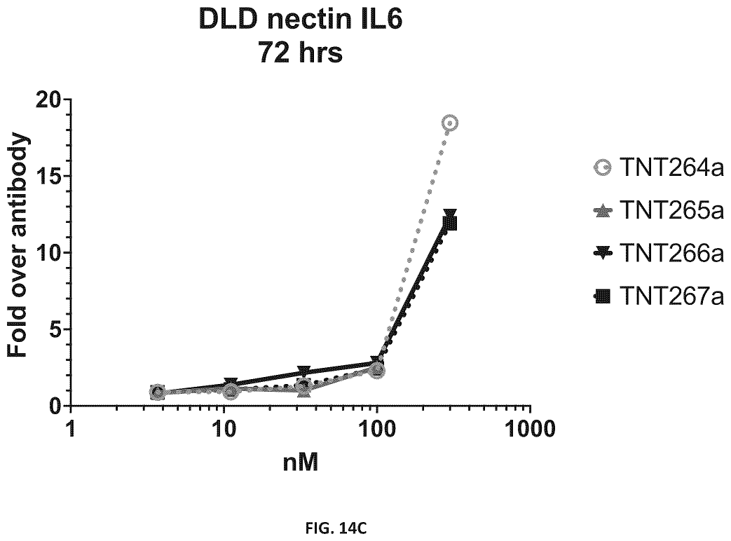

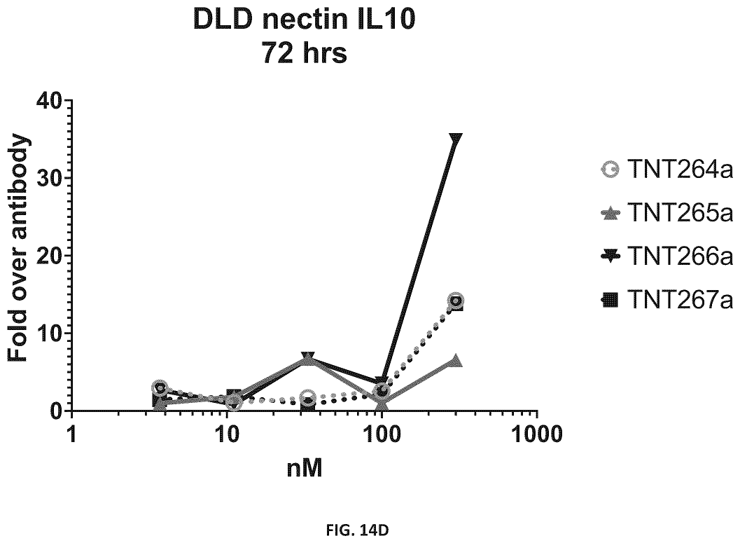

As shown in FIGS. 14A-D, the data showed enhanced cytokine secretion upon nectin-4 CpG conjugates engagement with nectin-4 positive tumor cells and PBMCs for IFNα2, IFNγ, IL6, and IL10. The antibodies tested were TNT-203, TNT-207, TNT-208, and TNT-210, and their corresponding conjugates tested were TNT-264a, TNT-265a, TNT-266a, and TNT-267a, respectively. In all cases, the conjugates showed marked enhancement as compared to their unconjugated antibody counterparts. Data in FIGS. 14A-D is presented as fold activity of conjugate over the corresponding unconjugated nectin-4 antibody.

### Example 15: Anti-Tumor Activity of Nectin-4 Antibody Conjugates

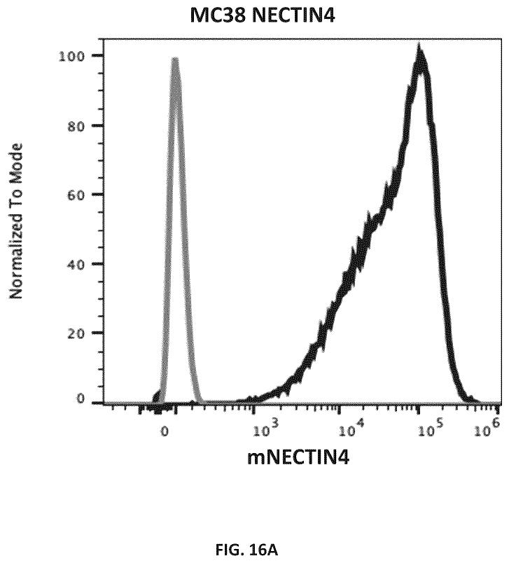

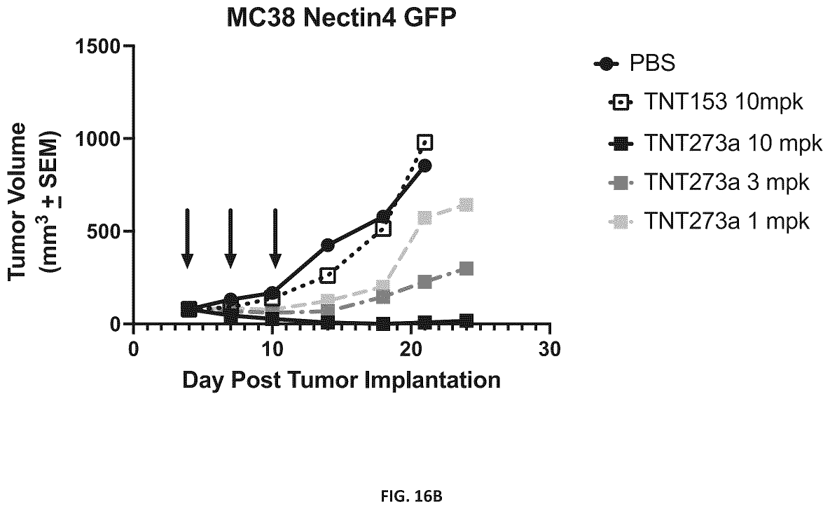

MC38 tumor cells expressing mouse nectin-4 (MC38 nectin-4) were generated by retroviral transduction and nectin-4 expressing cells were sorted and expanded. Nectin-4 expression in the transduced MC38 tumor cells was assessed prior to implantation with TNT208 conjugated with AF647 (Thermo Scientific) per manufacturer's protocol (FIG. 16A). MC38 nectin-4 mouse colon carcinoma cells were cultured in Complete DMEM (DMEM+Glutamax+10% FBS (Gibco)) at 37° C. and 5% CO2. Cells were detached with Trypsin 0.25% (Gibco) and washed twice with DMEM (Gibco). Cells were resuspended at 20E6/mL in DMEM and kept on ice until use. 100 uL of suspended cells were subcutaneously implanted into the right flank of 6-week-old female C57BL/6 mice (Charles River). Tumor size was measured and recorded twice a week with calipers starting 4 days post implantation until duration of the study. Tumor volume was calculated using the following formula: (length×width×width)/2. Once tumors reached an average of 80 mm³, approximately 4 days post implantation, mice were randomized by tumor size and treatments were initiated. Nectin-4-antibody conjugate in mouse IgG2a (TNT273a, which comprises TNT153 conjugated to CpG4523 (SEQ ID NO:499)) was administered intraperitoneally 3 doses 3 days apart at 1, 3 or 10 mg/kg. Mice whose tumors exceeded 2,000 mm³ or exhibited any signs of distress at any time during the study were euthanized humanely as per IACUC-approved animal protocols. The CpG4523 is tucgtcgtgacgtt-c3 (SEQ ID NO:499), where bases 1-5 are linked by phosphorothioate linkages, bases 5-6 are linked by phosphotriester linkages, bases 6-14 are linked by phosphorothioate linkages, base 2 is iodo -uridine, and base 14 is modified by c3.

As shown in FIG. 16A, nectin-4 expression is heterogeneous on transduced MC38 tumor cells (left peak denotes unstained cells). In FIG. 16B, systemic administration of nectin-4-antibody conjugate (TNT273a) exhibited potent, dose-dependent single agent activity in comparison to unconjugated nectin-4-antibody (TNT153) in a MC38 nectin-4 mouse model. Tumor eradication was observed at 3 and 10 mg/kg for 2 out of 4 or 3 out of 5 mice respectively. The eradication of tumors expressing varying levels nectin-4 expression implies that nectin-4 antibody conjugate can target a range of nectin-4 expressing tumors, including tumor expressing low/medium levels of nectin-4. This can occur either directly and/or indirectly by triggering an immune response that can target tumor cells expressing low/medium levels of nectin-4.

### Example 16: T Cell Activation by Nectin-4 Antibody Conjugates

Trima residual (VITALANT™) was diluted 1:2 with phosphate-buffered saline (PBS). Diluted blood was underlaid with 15 mL Ficoll-Paque (GE Healthcare) and tubes were centrifuged for 30 minutes at 400×g. PBMCs were collected from the interface, resuspended, and washed in FACS buffer (PBS with 0.5% bovine serum albumin; Gibco). After one wash, PBMCs were resuspended in Complete RPMI1640 (RPMI1640+10% FBS; Gibco). PBMCs were immediately placed onto a 96-well format (at 0.5E6 cells/well) in Complete RPMI1640. DLD-1 tumor cells, which were transduced to overexpress nectin-4 and GFP, were added to the PBMCs at a 20:1 (effector:target ratio; about 0.025E6 tumor cells/well). Three-fold serial dilutions of nectin-4 antibody conjugates, 300 nM to 0.41 nM, were added to the cells and incubated at 37° C. under 5% CO2 for 18 hours. After incubation, cells were pelleted by centrifugation for five minutes at 400×g and stained at 4° C. in Fixable Viability Dye eFluor 780 (Thermo Fisher) diluted 1:4000 in PBS. Cells were centrifuged and stained at 4° C. in FACS buffer for 30 minutes containing FcR Blocking Reagent (Miltenyi Biotec), anti-CD14, anti-CD 11c, anti-CD3, anti-CD19, anti-CD56, anti-CD16, anti-HLADR, anti-CD40, anti-CD86, and anti-CD69 (ThermoFisher, Biolegend). Cells were centrifuged and washed twice in FACS buffer and fixed in 0.5% paraformaldehyde. Cells were analyzed on Attune NxT Flow Cytometer (ThermoFisher), with subsequent data analysis by Flowjo 10.7. Dead cells were excluded by gating on the eFluor 780-negative population. T cells were identified as CD3⁺ cells. T cell activation levels were assessed by % increase in median fluorescent intensity over background.

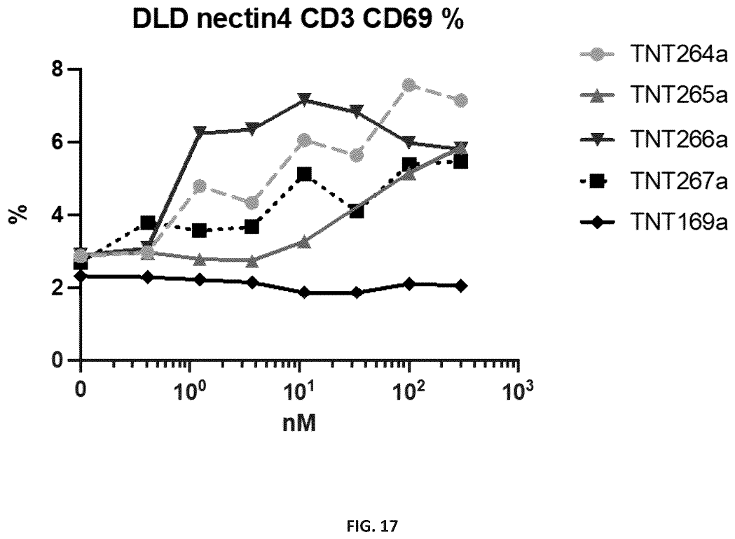

The conjugates tested were TNT264a, TNT265a, TNT266a, TNT267a, and TNT169a. As shown in FIG. 17, all the conjugates with the exception of TNT169a triggered robust induction of CD69 expression on T cells.

### Example 17: NK Cell Activation by Nectin-4 Antibody Conjugates

Trima residual (VITALANT™) was diluted 1:2 with PBS. Diluted blood was underlaid with 15 mL Ficoll-Paque (GE Healthcare) and tubes were centrifuged for 30 minutes at 400×g. PBMCs were collected from the interface, resuspended, and washed in FACS buffer. After one wash, PBMCs were resuspended in Complete RPMI. PBMCs were then immediately placed onto a 96-well format (at 0.5E6 cells/well) in Complete RPMI1640. DLD-1 tumor cells, which were transduced to overexpress nectin-4 and GFP, were added to the PBMCs at a 20:1 effector:target ratio (about 0.025E6 tumor cells/well). Three-fold serial dilutions, from 300 nM to 0.41 nM, of nectin-4 antibody conjugates were added to the cells and incubated at 37° C. under 5% CO2 for 18 hours. Cells were then pelleted by centrifugation for five minutes at 400×g and stained at 4° C. in Fixable Viability Dye eFluor 780 (ThermoFisher) diluted 1:4000 in PBS. Cells were centrifuged and stained at 4° C. in FACS buffer for minutes containing FcR Blocking Reagent (Miltenyi Biotec), anti-CD14, anti-CD11c, anti-CD3, anti-CD19, anti-CD56, anti-CD16, anti-HLADR, anti-CD40, anti-CD86, and anti-CD69 (ThermoFisher, Biolegend). Cells were centrifuged and washed twice in FACS buffer. Cells were then processed for intracellular staining using Transcription factor fixation/permeabilization concentrate and diluent (eBioscience). Briefly, cells were incubated in fresh fixation buffer by mixing 1 part of fixation/permeabilization concentrate with 3 parts of fixation permeabilization diluent. Samples were incubated for 30-60 minutes at 4° C. and protected from light. Samples were then centrifuged at 600×g for 5 minutes at room temperature. Pellets were resuspdended in 1× permeabilization buffer followed by two rounds of washes and centrifugation at 600×g for 5 minutes at room temperature. Pellets were resuspended in 100 μL of permeabilization buffer and stained with anti-IRF7 for 60 minutes at room temperature. Cells were centrifuged and washed twice in FACS buffer and fixed in 0.5% paraformaldehyde. Cells were analyzed on Attune NxT Flow Cyotometer (ThermoFisher), with subsequent data analysis by Flowjo 10.7. Dead cells were excluded by gating on the eFluor 780-negative population. NK cells were identified as CD56⁺CD3⁻cells. NK cell activation levels were assessed by median fluorescent intensity.

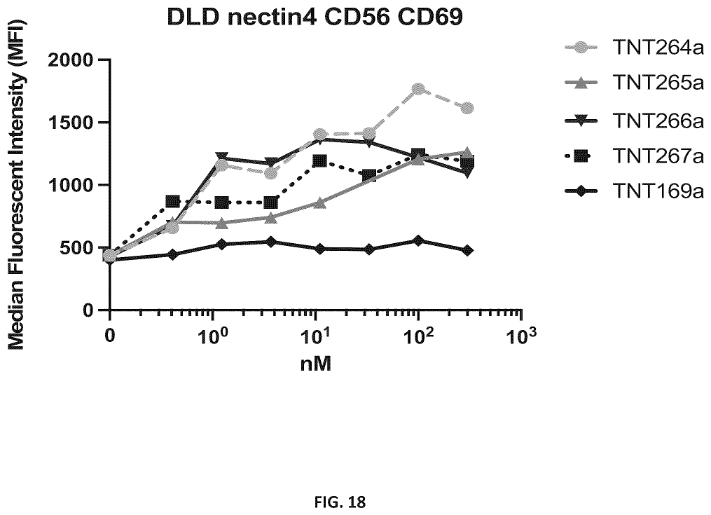

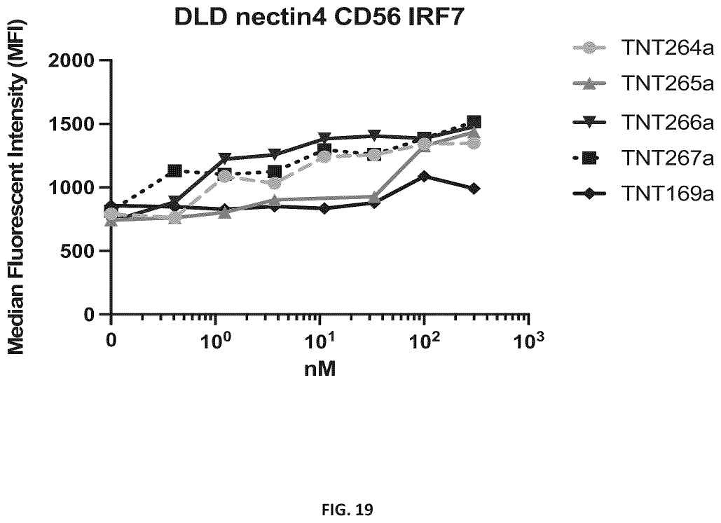

The conjugates tested were TNT264a, TNT265a, TNT266a, TNT267a, and TNT169a. As shown in FIG. 18, all conjugates except TNT169a triggered robust induction of CD69 expression on NK cells. As shown in FIG. 19, all conjugates triggered activation of IRF7 expression by NK cells, with the exception of TNT265a and TNT169a which showed moderately lesser activation.

### Example 18: Anti-Tumor Activity of Nectin-4 Antibody Conjugates

MC38 tumor cells expressing mouse nectin-4 (MC38-nectin-4) were generated by retroviral transduction and sorting for nectin-4 expression. MC38-nectin-4 cells were cultured in Complete DMEM (DMEM+Glutamax+10% FBS) at 37° C. at 5% CO2. Cells were detached with 0.25% trypsin (Gibco) and washed twice with DMEM. Cells were resuspended at 20E6 cells/mL in DMEM and kept on ice until use. 100 μL of suspended cells were subcutaneously implanted into the right flank of 6-week-old female C57BL/6 mice (Charles River). Tumor size was measured and recorded twice a week with calipers starting 4 days post implantation until duration of the study. Tumor volume was calculated using the following formula: (length×width×width)/2. Once tumors reached an average of 70 mm³, approximately 4 days post implantation, mice were randomized by tumor size and treatments were initiated. Nectin-4 antibody conjugates in mouse IgG2a (TNT302a, TNT303a, TNT304a, and TNT273a) were administered intraperitoneally 3 doses 3 days apart at 3 mg/kg. Mice whose tumors exceeded 2,000 mm³ or exhibited any signs of distress at any time were euthanized humanely as per IACUC-approved animal protocols.

The conjugates tested were TNT302a, TNT303a, TNT304a, and TNT237a. The oligonucleotide conjugated to the antibodies, CpG4523, is tucgtcgtgacgtt-c3 (SEQ ID NO:499), where bases 1-5 are linked by phosphorothioate linkages, bases 5-6 are linked by phosphotriester linkages, bases 6-14 are linked by phosphorothioate linkages, base 2 is iodo -uridine, and base 14 is modified by c3.

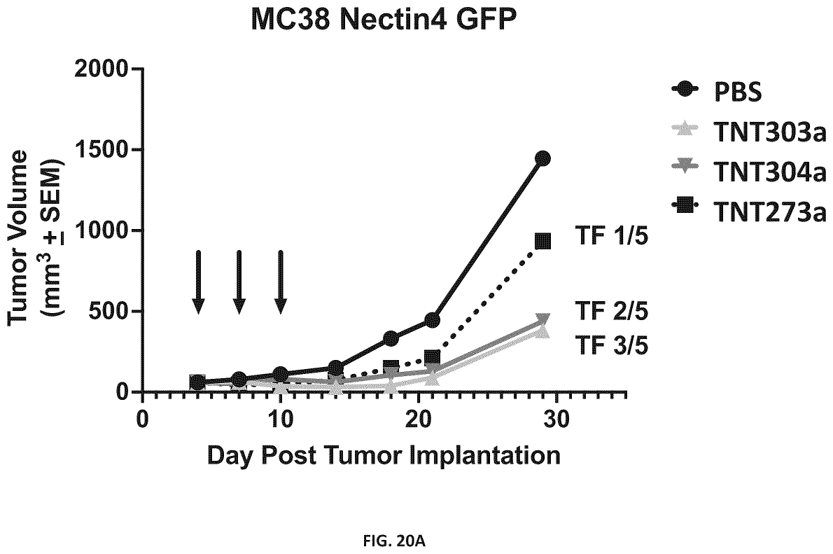

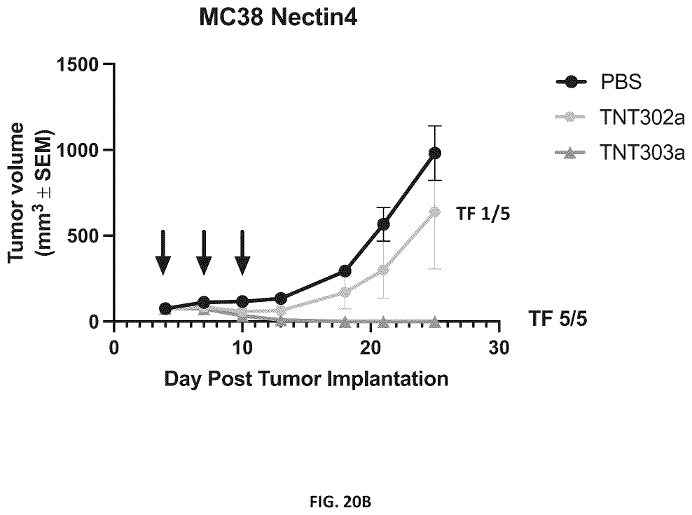

As shown in FIGS. 20A and 20B, nectin-4 antibody conjugates exhibited potent single-agent activity in a MC38 nectin-4 mouse model. As shown in FIG. 20A, both TNT303a and TNT304a exhibited superior single agent activity over TNT273a. Tumor eradication was observed at the 3 mg/kg dose. For example, at the final time point, 2 out of 5 mice treated with TNT304a were tumor free (TF 2/5) and 1 out of 5 mice treated with TNT273a were tumor free (TF 1/5). In another experiment and as shown in FIG. 20B, TNT303a exhibited superior activity compared with TNT302a, with complete tumor eradication and all treated mice were tumor free (TF 5/5).

### Example 19: Generation of Additional Nectin-4 Antibodies

Hybridomas were generated using adapted protocols described by Kohler and Milstein (Nature, 1975; 256:495-497). Briefly, AMM -XKL transgenic mice (Ablexis, California, United States) were immunized weekly with 50 mg of human nectin-4 ECD (designated TNT-175) mixed with in-house TLR agonist cocktail comprised of ODN 1826, Poly(I:C), resiquimod, and MPL® (monophosphoryl lipid A). Immunization routes included inguinal, back of the neck, base of tail subcutaneous as well as hock and intraperitoneal. Mice were bled monthly until desired titers were reached. Immune cells were collected and pooled from spleen and draining lymph nodes of 3 mice. CD138 expressing cells were isolated from this pool of cells using mouse CD138 positive selection kit (STEMCELL Technologies, Vancouver, Canada) as per manufacturer's protocol. Isolated cells were fused with mouse myeloma cells (ATCC CRL2016™) using ECM™ 2001 electroporation system (BTX, Holliston, MA, United States) method as described by STEMCELL Technologies. Fused cells were centrifuged, resuspended in semi-solid hypoxanthine, aminopterin and thymidine (HAT) selection media (STEMCELL Technologies, Vancouver, Canada) mixed with 10 mg/mL goat anti mouse IgG Fc FITC (Jacksonlmmuno, West Grove, PA, United States) and plated into NUNC™ OMNITRAYS™ (ThermoFisher, Waltham MA, United States). Plates were incubated at 37° C. with 7% CO2 for one week. Plates were imaged and fluorescent clones were picked into media E (STEMCELL Technologies) using the CLONEPIX® 2 colony picker system (Molecular Devices, San Jose, CA, United States). Picked clones were incubated at 37° C. with 7% CO2 until confluent.

Supernatant from picked clones was screened for binding to human nectin-4 ECD (TNT-175) via direct ELISA. Positive clones were counter screened against HIS tag and screened for cross reactivity to mouse nectin-4 (TNT-250). Positive clones were re-arrayed and further characterized via SPR.

Variable regions of confirmed hybridoma clones were amplified using a modified 5′RACE protocol. Briefly, RNA was isolated using PURELINK™ Pro 96 purification kit (Invitrogen, Waltham, MA, United States) according to manufacturer's protocol. All reactions were kept on ice during set up. Reverse transcription was executed using MAXIMA™ H Minus Reverse Transcriptase kit (Thermo Fisher) according to manufacturer's instructions with addition of gene specific primers in the constant domains of the variable mouse heavy and light chains. PolyA tail was added to the synthesized cDNA via Terminal Deoxynucleotidyl Transferase (Thermo Scientific) according to manufacturer's instructions, utilizing a lock-docking primer. Next, the variable region cDNA was amplified using a forward Oligo dT primer and nested reverse primer located in the constant domain of either heavy or the light chain region. Amplification reaction included 1 uL of synthesized cDNA, 1 uL 10 uM forward primer, 1 uL 10 uM reverse primer, 22 uL H₂O and 25 uL DreamTaq Green PCR Master Mix (Thermo Scientific). PCR was performed according to following thermocycler conditions: 1 cycle of 95° C. for 2 minutes; 30 cycles of 94° C. for 30 seconds, 56° C. for 30 seconds, 72° C. for 1 minute followed by 1 cycle of 72° C. for 10 minutes and 1 cycle of 4° C. forever. Amplicons were confirmed via electrophoresis using 2% Agarose E-gel (Invitrogen) and visualized under UV light. Amplicons were purified using MinElute 96UF PCR Purification Kit (Qiagen) and resuspended in 50 uL molecular grade water (MilliQ). Amplicons were sent out for Sanger sequencing to Elim Labs (Hayward, CA) with nested reverse sequencing primers. Sequences were analyzed using Geneious Biologics (Biomatters, Auckland, New Zealand). Unique sequences were identified and selected for further characterization.

Clones selected include those designated as TNT-317, TNT-318, TNT-320, TNT-321, TNT-322, TNT-323, TNT-324, TNT-327, TNT-328, TNT-330, TNT-331, TNT-332, TNT-333, TNT-334, TNT-335 and TNT-336. The VH, VL, and CDR sequences are listed in Tables 7-11, above.

### Example 20: Antibody Characterization—Affinities Measurements and Epitope Binning

This example compares nectin-4 antibodies of the examples and determines which epitope they recognize of nectin-4 and their binding to human, mouse, and cynomolgus nectin-4.

K_{D} affinity determination: All experiments were performed at 25° C. using a Biacore 8K high throughput, high-sensitivity SPR system (Cytiva, Global Life Sciences Solutions USA LLC, Marlborough, MA) equipped with S⁻type sensor chips. The running buffer was 10 mM HEPES, pH 7.4, 150 mM NaCl, 3 mM EDTA, 0.05% (v/v) Surfactant P20 (HBS-EP+). The interactions of various anti-nectin-4 monoclonal antibodies with recombinant human nectin-4-ECD-6×His protein (TNT-175) were analyzed by flowing nectin-4-ECD over Protein A captured anti-nectin-4 antibody on Biacore Series S Protein A Sensor Chip. The nectin-4 analytes were injected in either single-cycle kinetics or multi-cycle kinetics mode at nominal starting concentrations of 300 nM or 900 nM with 3-fold serial dilutions. Association times were monitored for 120 s, and dissociation times were monitored 1800 s at 30 μL/min flow rate. The surfaces were regenerated with 10 mM glycine at pH1.7 or 75 mM phosphoric acid at pH1.6 for two pulses of 15 s at 30 μL/min flow rate. The analytes tested were human nectin-4 ECD (TNT-175), human nectin-4 IgV (TNT-252), mouse nectin-4 ECD (TNT-250) or mouse nectin-4 ECD (TNT-176), and cyno nectin-4 ECD (TNT-255).

The data were processed and analyzed with Biacore 8K Evaluation Software Version 3.0.12.15655 (Cytiva, Global Life Sciences Solutions USA LLC, Marlborough, MA). Reference responses from flow cell 1 were subtracted from the active responses from flow cell 2 to obtain the subtracted data (2-1). The responses from the nearest buffer blank injection were then subtracted from the reference subtracted data (2-1) to yield double-referenced data. These double-reference data were fit to a simple 1:1 Langmuir binding model with mass transport to determine the apparent association (ka) and dissociation rate constants (kd). The apparent equilibrium dissociation constant or affinity constant was then calculated based on their ratio as (K_{D}=kd/ka).

Epitope binning: SPR binning experiments were performed by immobilizing various nectin-4 antibodies on Biacore CM5 chip via amine coupling method as ligand and using 300 nM antigen human nectin-4 ECD (TNT-175) or 300 nM TNT-175 plus 3 uM competing antibody as analyte in solution. The ligand antibody and the competing antibody were considered to be in the same bin when positive binding was observed only in the presence of human nectin-4 ECD alone while not in the presence of high concentration of competing antibody.

Table 24, below, shows the binding affinities to human nectin-4 ECD, human nectin-4 IgV domain, mouse nectin-4 ECD and cyno nectin-4 ECD. In addition, the epitope binning results are shown. The apparent affinity K_{D} of the antibodies towards human nectin-4 ECD were from ˜1 nM to 260 nM. All antibodies bound to human nectin-4 IgV domain with the exception of TNT-317, TNT-318, TNT-322, TNT-323, TNT-328, TNT-331, TNT-332 and TNT-333. All antibodies bound to cyno nectin-4 ECD except TNT-336. Most antibodies bound to mouse nectin-4 ECD except TNT-317, TNT-318, TNT-320, TNT-321, TNT-322, TNT-323, TNT-324, TNT-327, TNT-328, TNT-330, TNT-331, TNT-332, TNT-333, TNT-334, TNT-335 and TNT-336.

TABLE 24
Affinities and epitope binning of nectin-4 antibodies.

| | Affinities (K_{D}, M) | | | | |
|---|---|---|---|---|---|
| | Human | Human | Mouse | Cyno | |
| | nectin-4 | nectin-4 | nectin-4 | nectin- | |
| Antibody | ECD | IgV | ECD | 4 ECD | Binning |
| TNT-201 | 6.05E−09 | 4.39E−09 | 2.09E−08 | 1.67E−08 | Bin1 |
| TNT-202 | 1.48E−08 | 1.34E−08 | 2.34E−08 | 2.56E−08 | Bin1 |
| TNT-203 | 1.01E−07 | 1.38E−07 | 1.96E−07 | 1.72E−07 | Bin1 |
| TNT-204 | 2.08E−09 | 6.82E−09 | 4.34E−09 | 1.02E−08 | Bin1 |
| TNT-205 | 5.39E−08 | 7.53E−08 | 8.20E−07 | 7.27E−07 | Bin1 |
| TNT-206 | 1.77E−08 | 1.87E−08 | 1.87E−08 | 2.35E−08 | Bin1 |
| TNT-207 | 1.37E−09 | 1.77E−09 | 5.07E−09 | 4.19E−09 | Bin1 |
| TNT-208 | 4.56E−09 | 5.43E−09 | 1.86E−08 | 1.19E−08 | Bin1 |
| TNT-209 | 1.75E−08 | 2.89E−08 | 3.31E−07 | 5.50E−08 | Bin1 |
| TNT-210 | 1.33E−08 | 1.75E−08 | 1.50E−08 | 3.67E−08 | Bin1 |
| TNT-211 | 2.43E−08 | 2.43E−08 | 6.84E−08 | 4.89E−08 | Bin1 |
| TNT-154/ | 8.69E−09 | 9.88E−09 | low | 4.05E−08 | Bin1 |
| TNT-212 | | | | | |
| TNT-317 | 1.57E−08 | NB | NB | 1.83E−08 | Bin2 |
| TNT-318 | 2.60E−07 | NB | NB | 2.11E−07 | — |
| TNT-320 | 1.84E−08 | 9.91E−09 | NB | 3.59E−08 | Bin1 |
| TNT-321 | 1.40E−08 | 1.16E−08 | NB | 5.60E−08 | Bin1 |
| TNT-322 | 5.79E−08 | NB | NB | 5.15E−08 | — |
| TNT-323 | 9.00E−09 | NB | NB | 8.67E−09 | Bin2 |
| TNT-324 | 6.45E−08 | 3.07E−08 | NB | 2.44E−08 | Bin1 |
| TNT-327 | 3.63E−09 | 1.64E−09 | NB | 2.85E−09 | Bin3 |
| TNT-328 | 1.59E−09 | NB | NB | 1.78E−09 | Bin2 |
| TNT-330 | 1.64E−08 | 7.30E−09 | NB | 1.37E−08 | Bin3 |
| TNT-331 | 8.74E−08 | NB | NB | 3.28E−08 | Bin2 |
| TNT-332 | 7.60E−09 | NB | NB | 7.20E−09 | Bin4 |
| TNT-333 | 6.18E−09 | NB | NB | 7.43E−09 | Bin2 |
| TNT-334 | 7.15E−08 | 4.63E−08 | NB | 9.53E−08 | Bin1 |
| TNT-335 | 1.83E−09 | 9.85E−10 | NB | 1.28E−09 | Bin1 |
| TNT-336 | 1.11E−07 | 1.04E−07 | NB | NB | — |
| TNT-337 | 2.17E−07 | 1.95E−07 | 3.18E−07 | 2.86E−07 | Bin1 |
| TNT-412 | 7.66E−09 | — | 1.36E−08 | 6.43E−09 | Bin1 |
| TNT-412 | 7.54E−09 | — | 1.31E−08 | 5.99E−09 | Bin1 |
| (non- | | | | | |
| fucosylated) | | | | | |

NB = no binding;
LB = low binding;
“—” = not done

### Example 21: Cell Binding to Human Nectin-4 and Human Nectin

This example shows the binding of the antibodies described above to various nectin-4-expressing cell lines or nectin-1-expressing cell lines.

Cell binding of nectin-4 antibodies: nectin-4 positive HT-1376, T47-D, and SKBR3 cells were obtained from ATCC and cultured according to their protocols. DLD-1 was transduced to overexpress nectin-4 and GFP and cultured according to ATCC protocols. Cells were detached with TrypLE (Gibco) and washed by centrifugation for five minutes at 400×g with FACS buffer (Phosphate Buffered Saline, Gibco+2% Fetal Bovine Serum, Gibco). Cells were resuspended in FACS buffer, plated in 96-well format (0.25 e6/well) then pelleted by centrifugation for five minutes at 400×g. Cells were stained at 4° C. in Fixable Viability Dye eFluor 506 (eBioscience) diluted 1:2,000 in PBS. Nectin-4 antibodies were labelled with AlexaFluor-647 (AF647) using Protein Labeling Kit (Invitrogen) according to manufacturers' protocol. In brief, 50 μg of antibody in PBS were incubated with reactive dye reagents under gentle agitation at room temperature for 1 hour. Unincorporated dyes were removed by size exclusion chromatography using purification resins and columns provided in the kits. Cells were centrifuged and stained at 4° C. in FACS buffer for 60 minutes containing FcR Blocking Reagent (Miltenyi Biotec) and AF647 conjugated Nectin-4 antibodies. Cells were centrifuged, washed twice with FACs buffer and fixed in 0.5% paraformaldehyde. Cells were analyzed on Attune NxT Flow Cytometer (ThermoFisher), with subsequent data analysis by Flowjo 10.7 (BD). Dead cells were excluded by gating on the eFluor 506-negative population. Binding was assessed by median fluorescent intensity.

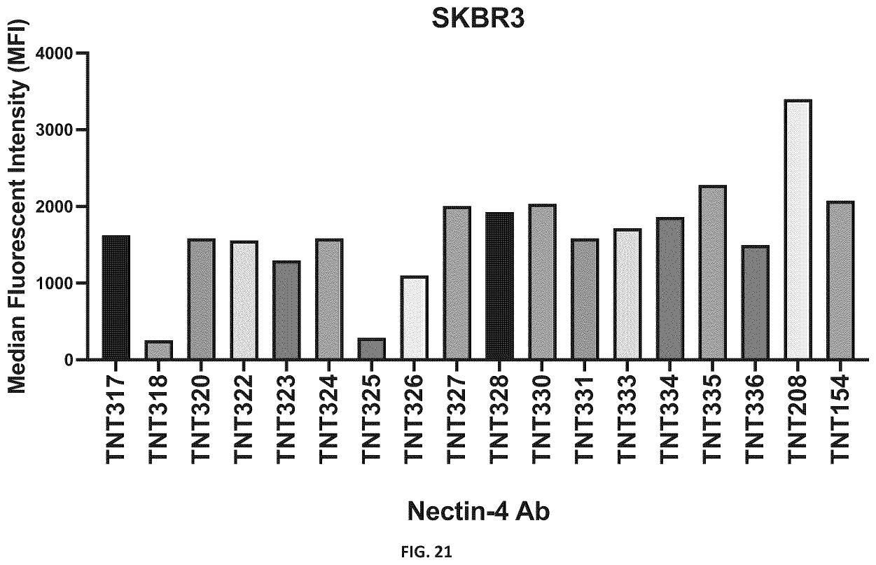

All nectin-4 antibodies bound to endogenously expressing nectin-4 in the HT-1376, T47-D, and SKBR3 cell lines, with the exception of TNT-318 and TNT-325 which exhibited poorer binding as compared to the other antibodies. Results for SKBR3 are shown in FIG. 21.

Nectin-4 activation specificity on nectin-1 cells: MM1R cells, which are nectin-1 positive and nectin-4 negative, were obtained from ATCC and cultured according to their protocols. Nectin-1 negative DLD-1 was transduced to overexpress nectin-4 and GFP and cultured according to ATCC protocols. Plateletpheresis Leukoreduction Chamber (LRS chamber) was received from Vitalant and diluted 1:3 with PBS. Diluted blood was underlaid with 12 mL Ficoll-Paque. Tubes were centrifuged for 30 minutes at 400×g. PBMCs were collected from the interface, resuspended and washed in FACS buffer (PBS with 0.5% BSA). After one wash, PBMCs were resuspended in Complete RPMI1640 (RPMI1640+10% FBS). PBMCs were immediately plated onto a 96-well format (0.5 e6/well) in Complete RPMI. Tumor cells (0.025E6/well) from MM1R labeled with CellTrace CFSE (ThermoFisher) or DLD-1 transduced to overexpress nectin-4 and GFP were added to PBMC at 20:1 (effector to target). Three-fold serial dilutions were added to the cells from 300 nM to 0.41 nM of nectin-4 antibody alone and antibody-CpG conjugates at 37° C. under 5% CO2 for 18 hours. Cells were pelleted by centrifugation for five minutes at 400×g and stained at 4° C. in Fixable Viability Dye eFluor 780 (ThermoFisher) diluted 1:4000 in PBS. Cells were centrifuged and stained at 4° C. in FACS buffer for 30 minutes containing FcR Blocking Reagent (Miltenyi Biotec), anti-CD14, anti-CD 11c, anti-CD3, anti-CD19, anti-CD56, anti-CD16, anti-HLADR, anti-CD69, anti-CD86 (ThermoFisher, Biolegend). Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (BD). Dead cells were excluded by gating on the eFluor 780-negative population. T cells were identified as CD3⁺ cells, monocytes were identified as CD3⁻CD19⁻CD14⁺ cells, and dendritic cells (DC) were identified as CD3⁻CD19-CD14⁻CD56⁻CD11C⁺ HLADR⁺ cells. T cell, monocyte, and DC activation levels were assessed by median fluorescent intensity.

The antibody-CpG conjugates were prepared using methods described in Example 10. TNT-266a was specific in activation of nectin-4 positive cells (DLD-1) with no activation observed in nectin-1 positive cells (MM1R). No activation was observed with the naked antibody TNT-208 in both DLD-1 and MM1R cell lines, as expected.

### Example 22: Antibody Internalization

This example shows the comparison of internalization between nectin-4 antibodies.

Internalization was first performed using endogenously expressed nectin-4 T47D cell line. Subsequently, a subset of antibodies was also tested using the exogenously transduced nectin-4 DLD cells. Antibodies designated TNT-154, TNT-208, TNT-317, TNT-318, TNT-327, TNT-328, TNT-330, TNT-333, and TNT-336 were labelled with AlexaFluor-647 (AF647) using Protein Labeling Kit (Invitrogen) according to manufacturers' protocol. In brief, 100 μg of an antibody in PBS were incubated with reactive dye reagents under gentle agitation at room temperature for 1 hour. Unincorporated dyes were removed by size exclusion chromatography using purification resins and columns provided in the kits. Human nectin-4 expressing cells, T47D, were sourced from ATCC and cultured according to their recommendations. DLD-1 was transduced to overexpress nectin-4 and GFP and cultured according to ATCC protocols. Cells were detached with TrypLE (Gibco) and washed by centrifugation for five minutes at 400×g with FACS buffer (Phosphate Buffered Saline, Gibco+1% Bovine Serum Albumin, Gibco). Cells were resuspended in FACS buffer and stained with 10 μg labeled nectin-4 antibody for 60 minutes at 4° C. Cells were washed twice with FACS buffer and resuspended in 1 mL FACS buffer per sample. Cells were kept on ice in dark until a 100 μL aliquot per sample tube was shifted to 37° C. under 5% CO2 incubator at time −3 hours, −2 hours, −1 hour, −30 minutes, −15 minutes, −5 and −0 minutes for internalization such that all timepoints are complete at the same time. Upon removal from incubator, cells were washed with FACS buffer then split into 2 aliquots with one resuspended in 100 μL Papain (Papain from papaya latex, Sigma) at 23.9 U/mL diluted in PBS (Gibco) and the other ½ resuspended in 100 μL PBS and incubated for 30 minutes at 37° C. Cells were then washed and stained for 30 minutes at 4° C. in Fixable Viability Dye eFluor 506 (eBioscience) diluted 1:2000 in PBS (Gibco). Cells were washed once more with FACS buffer and fixed in 0.5% paraformaldehyde. Cells were analyzed on Attune NxT Flow Cytometer (ThermoFisher), with subsequent data analysis by Flowjo 10.7 (BD). Dead cells were excluded by gating on the eFluor 506-negative population. Samples were assessed by median fluorescent intensity for each timepoint. The samples that were not incubated with papain were used as a “no internalization” control and thus considered to have 100% antibody-AlexaFluor-647 localized on cell surface. Cells from the −0 minute timepoint that were incubated at 37° C. with papain was used as negative control. Internalized antibody was expressed as a percentage calculated as the difference between the papain cleaved sample minus the negative control relative to the surface-localized antibody at 37° C. minus the negative control.

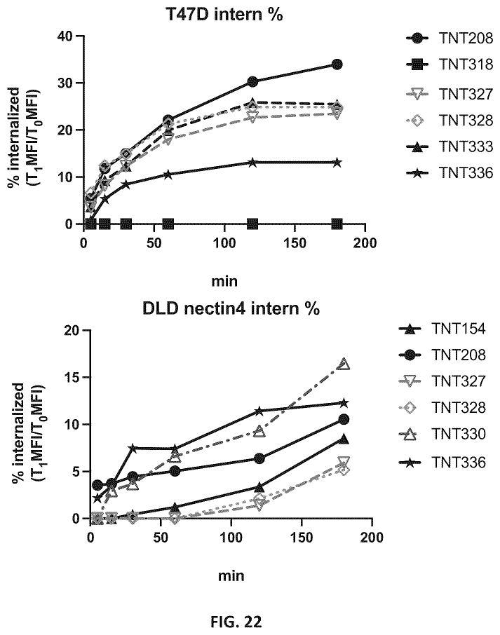

Results are shown in FIG. 22. All the tested nectin-4 antibodies internalize with varying degrees with both cell lines (T47D and nectin-4-DLD) except for TNT-318 and TNT-336 which exhibited poor internalization in T47D as compared to the other antibodies.

### Example 23: Immune Cell Activation with Nectin-4 Antibody Conjugates

This example demonstrates activation on immune cells by certain nectin-4 antibodies described above and the correlation of activity with the level of nectin-4 expression.

HT1376, OE19, and H292 cells expressing high, medium and low nectin-4, respectively, were obtained from ATCC and cultured according to their protocols. DLD-1 was transduced to overexpress nectin-4 and GFP and cultured according to ATCC protocols. Plateletpheresis Leukoreduction Chamber (LRS chamber) was received from Vitalant and diluted 1:3 with PBS. Diluted blood was underlaid with 12 mL Ficoll-Paque. Tubes were centrifuged for 30 minutes at 400×g. PBMCs were collected from the interface, resuspended and washed in FACS buffer (PBS with 0.5% BSA). After one wash, PBMCs were resuspended in Complete RPMI1640 (RPMI1640+10% FBS). Tumor cells (0.025E6/well) were labeled with CellTrace CFSE (ThermoFisher) and plated onto a 96-well format in complete RPMI media. Three-fold serial dilutions of nectin-4 antibody conjugates from 300 nM to 0.41 nM were added to the target cells and incubated at 37° C. for 20 minutes. Freshly isolated PBMCs (0.5 e6/well) in Complete RPMI were added to the wells at 20:1 (effector to target) and incubated at 37° C. under 5% CO2 for 18 hours. Cells were pelleted by centrifugation for five minutes at 400×g and stained at 4° C. in Fixable Viability Dye eFluor 780 (ThermoFisher) diluted 1:4000 in PBS. Cells were washed 1× with FACs buffer and centrifuged and stained at 4° C. in FACS buffer for 30 minutes containing FcR Blocking Reagent (Miltenyi Biotec), anti-CD14, anti-CD11c, anti-CD3, anti-CD19, anti-CD56, anti-CD16, anti-HLADR, anti-CD69, anti-CD86 (ThermoFisher, Biolegend). Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (BD). Dead cells were excluded by gating on the eFluor 780-negative population. T cells were identified as CD3⁺ cells, Monocytes were identified as CD3⁻CD19⁻CD14⁺ cells, NK cells were identified as CD3⁻CD19⁻CD56⁺ cells and dendritic cells (DC) were identified as CD3⁻CD19⁻CD14⁻CD56⁻CD11C⁺ HLADR⁺ cells. T, monocyte, NK and DC activation levels were assessed by median fluorescent intensity.

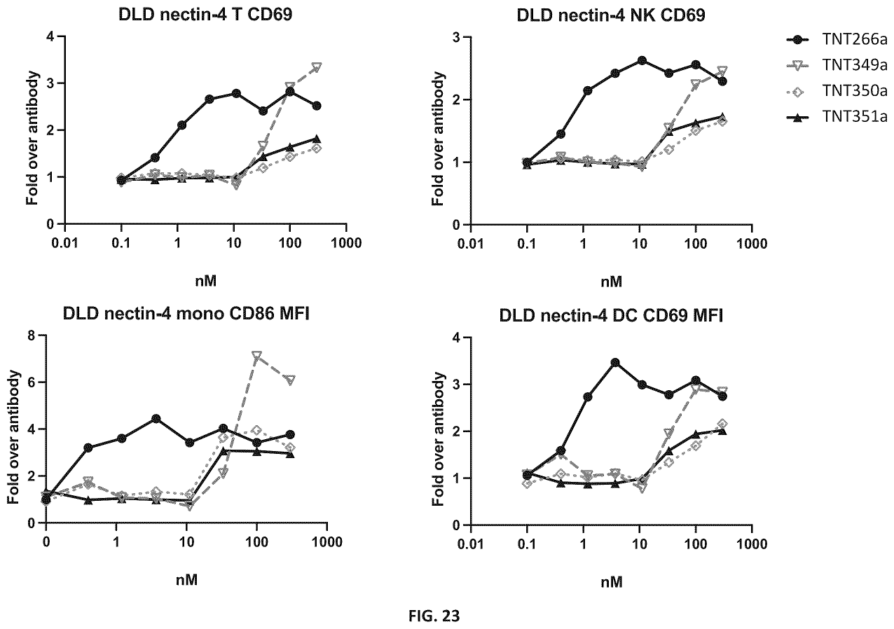

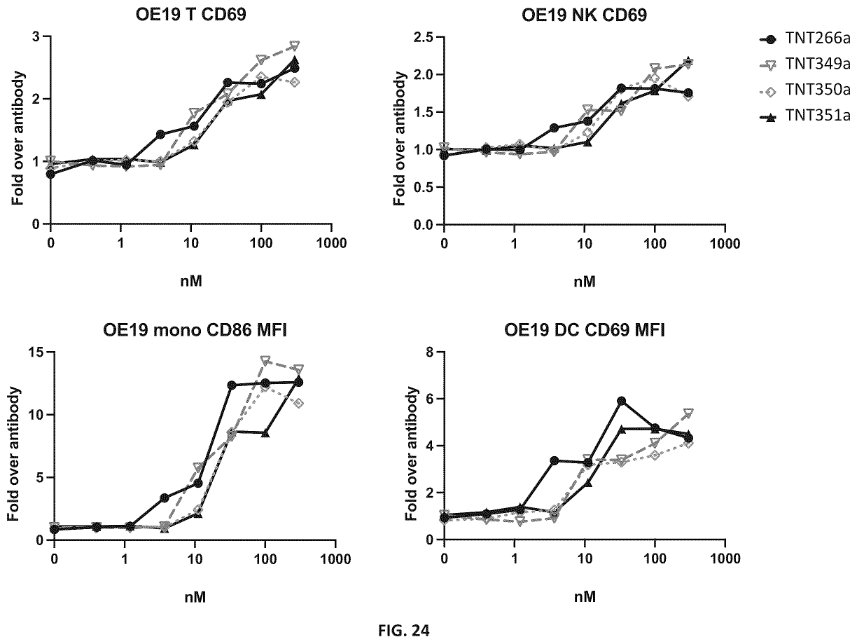

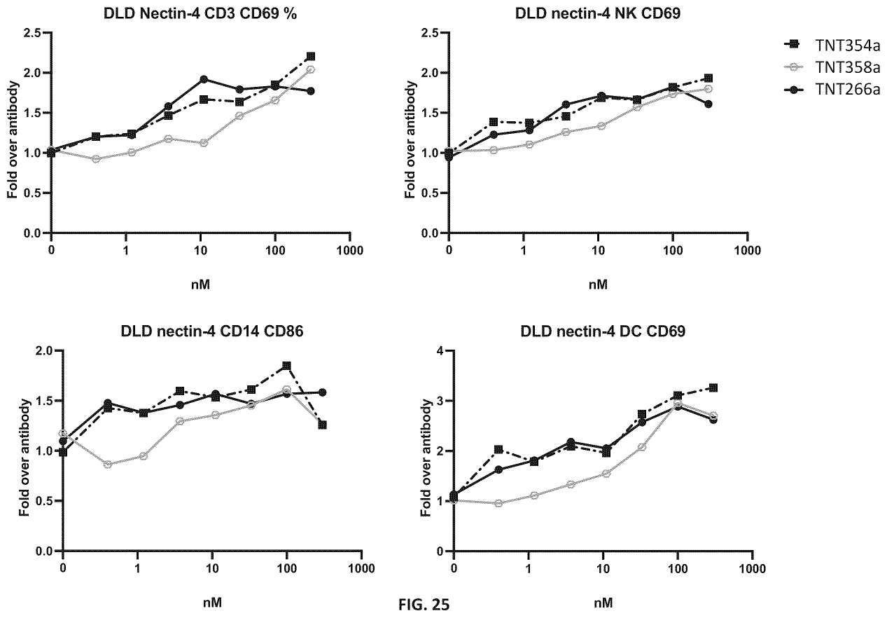

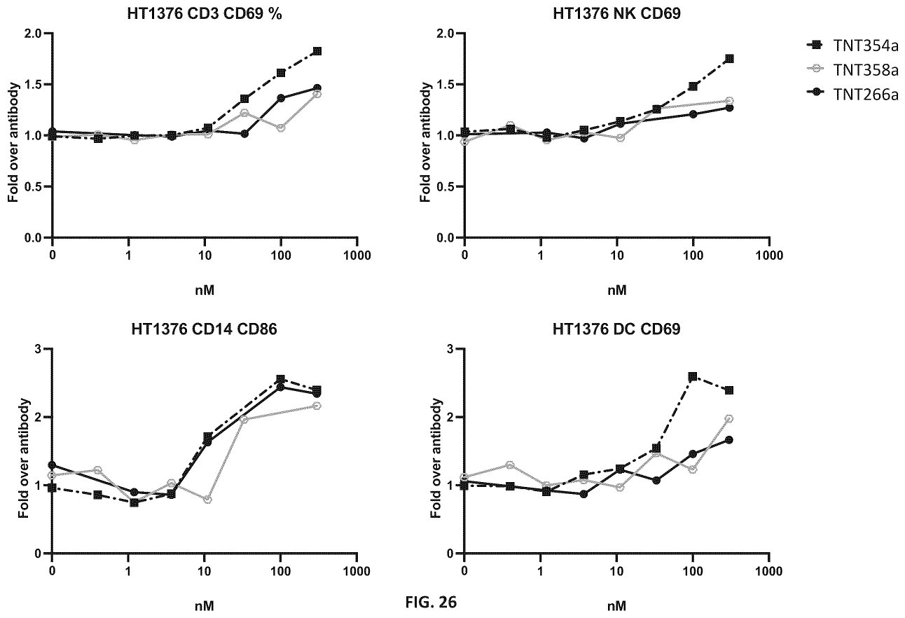

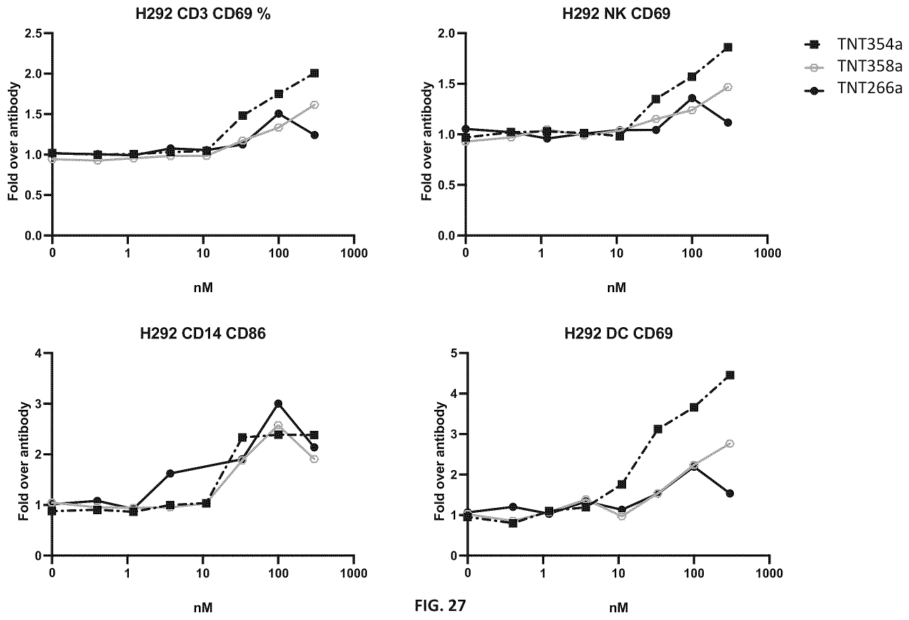

Results are shown in FIGS. 23-27. The activation of T-cell, NK, DC and monocytes by nectin-4-CpG conjugates was assessed in PBMC co-culture experiments with 4 cancer cell lines with varying ranges of nectin-4 expression (nectin-4-DLD>>HT1376>OE19>H₂₉₂). In the 4 cell lines tested, all nectin-4 CpG conjugated antibodies had increased activation over media for both innate and adaptive pathways based on the immune cells assessed. TNT-266a had higher levels of activation of T cells, NK cells, monocytes, and DCs compared to other conjugated antibodies in DLD-1 cells overexpressing nectin-4 (FIG. 23). Interestingly, the levels of activation of T, NK, monocytes, and DCs in co-culture experiments with OE19 are comparable between TNT266a, TNT349a, TNT350a and TNT351a (FIG. 24). TNT354a and TNT266a showed moderately higher levels of activation compared with TNT358a in all cell types (T, NK, monocytes, and DC) in the co-culture experiments using DLD-1 cells overexpressing nectin-4 (FIG. 25). Interestingly, TNT354a was observed to exhibit higher levels of activation of T, NK, and DC in co-culture experiments using both HT1376 and H292 compared to TNT-358a and TNT-266a (FIG. 26 and FIG. 27).

### Example 24: Comparison of Immune-Activating Conjugates

This example examines the mechanisms for immune activation by certain nectin-4 antibodies.

Comparison with a nectin-4-TLR8 conjugate. For comparator purposes, a TLR8 agonist (see structure immediately below) was synthesized for conjugation with an anti-nectin-4 antibody (TNT-337) expressed transiently in a 293 mammalian cell line. The antibody was purified using a standard Protein A purification method. The antibody-TLR8 agonist conjugate molecule (designated TNT-347xx) was generated by TCEP reduction of cysteines on the antibody at 37° C. for 1.5 h followed by incubation with 6-10 molar excess of the TLR8 agonist with the maleiamide moiety at room temperature for 3 to 6 h. The reaction was quenched by L-cysteine at RT for 30 min before desalted using Zeba spin columns. The final conjugate had a DAR of 2.5 based on MS analysis.

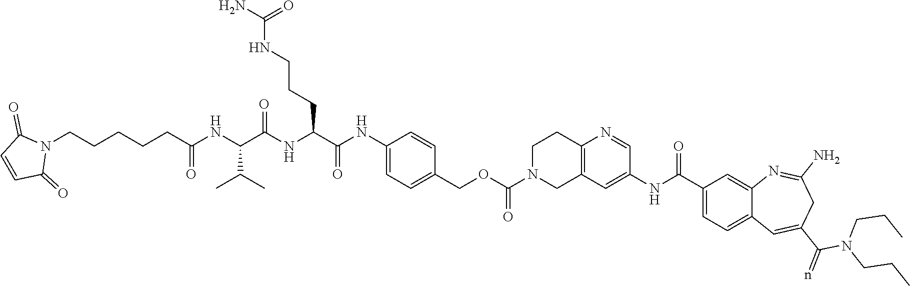

OE19 cells were obtained from ATCC and cultured according to their protocols. Plateletpheresis Leukoreduction Chamber (LRS chamber) was received from Vitalant and diluted 1:3 with PBS. Diluted blood was underlaid with 12 mL Ficoll-Paque. Tubes were centrifuged for 30 minutes at 400×g. PBMCs were collected from the interface, resuspended and washed in FACS buffer (PBS with 0.5% BSA). After one wash, PBMCs were resuspended in Complete RPMI1640 (RPMI1640+10% FBS). Tumor cells (0.025E6/well) were labeled with CellTrace CFSE (ThermoFisher) and plated onto a 96-well format in complete RPMI media. Three-fold serial dilutions of TNT-208, TNT-208-CpG conjugate, TNT-337, and TNT-347xx from 300 nM to 0.41 nM were added to the target cells and incubated at 37° C. for 20 minutes. Freshly isolated PBMCs (0.5 e6/well) in Complete RPMI were added to the wells at 20:1 (effector to target) and incubated at 37° C. under 5% CO₂ for 18 hours. Cells were pelleted by centrifugation for five minutes at 400×g and stained at 4° C. in Fixable Viability Dye eFluor 780 (ThermoFisher) diluted 1:4000 in PBS. Cells were washed 1× with FACs buffer and centrifuged and stained at 4° C. in FACS buffer for 30 minutes containing FcR Blocking Reagent (Miltenyi Biotec), anti-CD14, anti-CD11c, anti-CD3, anti-CD19, anti-CD56, anti-CD16, anti-HLADR, anti-CD123, anti-CD40, anti-CD69, and anti-CD86 (ThermoFisher, Biolegend). Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (BD). Dead cells were excluded by gating on the eFluor 780-negative population. T cells were identified as CD3⁺ cells, Monocytes were identified as CD3⁻CD19⁻CD14⁺ cells, NK cells were identified as CD3⁻CD19⁻CD56⁺ cells, plasmacytoid dendritic cells (pDCs) were identified as CD3⁻CD19⁻CD56⁻CD11C-CD123⁺ cells and dendritic cells (DCs) were identified as CD3⁻CD19⁻CD14⁻CD56⁻CD123⁻CD11C⁺ HLADR⁺ cells. T, monocyte, NK, pDC, and DC activation levels were assessed by median fluorescent intensity.

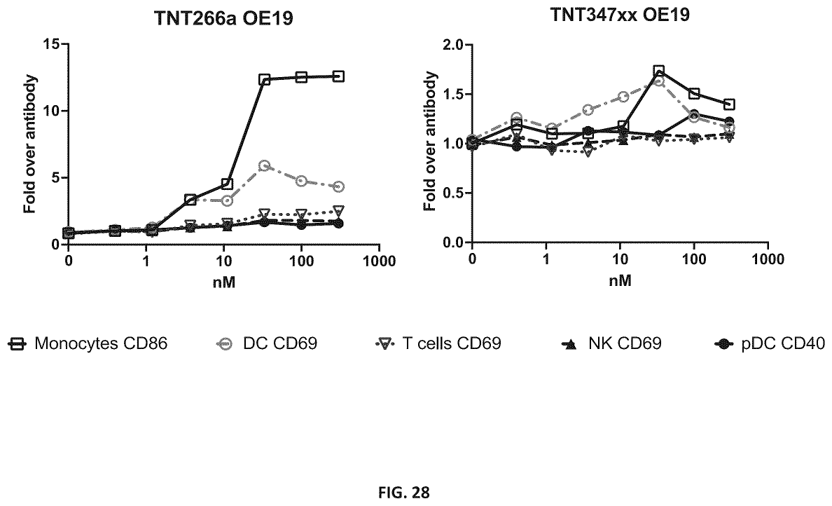

Results are shown in FIG. 28. Across multiple types of myeloid lineages as well as T and NK effector cells, TNT-266a had higher levels of activation in comparison to TNT-347xx TLR8-antibody conjugate in nectin-4 expressing OE19 cells.

Comparison with a nectin-4-antibody-MMAE conjugate. For comparison, a nectin-4-directed antibody-drug conjugate (ADC) comprised of a fully human anti-nectin-4 IgG1 kappa monoclonal antibody conjugated to the small molecule microtubule disrupting agent, monomethyl auristatin E, via a protease-cleavable maleimidocaproyl valine-citrulline linker (herein referred to as mc-vc-PAB-MMAE) was generated. The anti-nectin-4 antibody coding sequences were synthesized by ATUM (Newark, CA). The antibody TNT-212 protein was produced by transient expression in the 293 mammalian cell line. The antibody was purified using standard Protein A purification method. The vedotin linker-payload was conjugated to interchain cysteine residues that comprise the interchain disulfide bonds of the antibody to yield a product with a drug-to-antibody ratio of approximately 3.8:1. The conjugated ADC was designated TNT-381xx.

MC38 tumor cells expressing mouse nectin-4 and GFP (MC38 nectin-4) were generated by retroviral transduction and nectin-4 expressing cells were sorted and expanded. MC38 nectin-4 mouse colon carcinoma cells were cultured in Complete DMEM (DMEM+Glutamax+10% FBS (Gibco)) at 37° C. and 5% CO2. Cells were detached with Trypsin 0.25% (Gibco) and washed twice with DMEM (Gibco). Cells were resuspended at 20E6/mL in DMEM and kept on ice until use. 100 μL of suspended cells were subcutaneously implanted into the right flank of 6-week-old female C57BL/6 mice (Charles River). Tumor size was measured and recorded twice a week with calipers starting 4 days post implantation until duration of the study. Tumor volume was calculated using the following formula: (length×width×width)/2. Once tumors reached an average of 73 mm³, approximately 4 days post implantation, mice were randomized by tumor size and treatments were initiated. TNT-348a was administered intraperitoneally 3 doses 3 days apart at 10 or 3 mg/kg; TNT-381xx similarly was administered intravenously 3 doses 3 days apart at 10 mg/kg. Mice whose tumors exceeded 2,000 mm³ or exhibited any signs of distress at any time during the study were euthanized humanely as per IACUC-approved animal protocols.

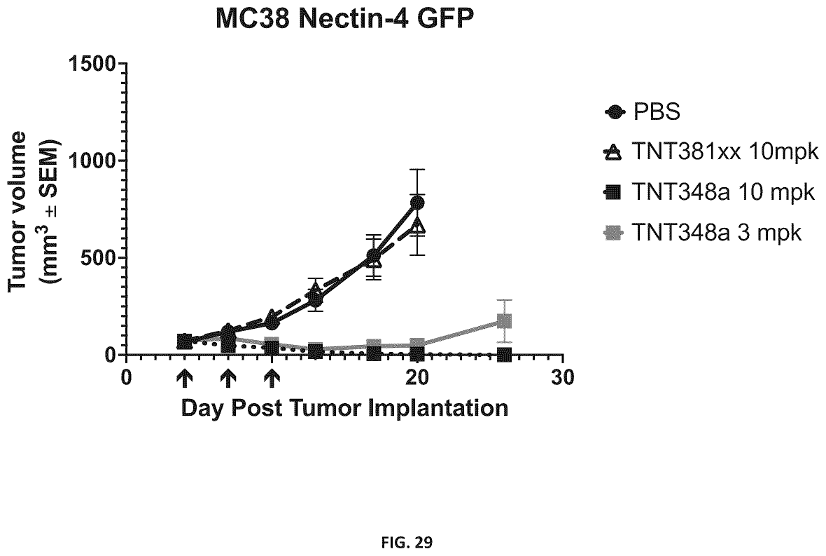

Results are shown in FIG. 29. Systemic administration of TNT-348a exhibited potent single agent activity in a MC38 nectin-4 mouse model. TNT-348a at both 10 and 3 mg/kg exhibited superior single agent activity over TNT-381xx which was administered at a single dose of 10 mg/kg.

### Example 25: Non-Fucosylated Nectin-4 Antibody Conjugates

A non-fucosylated version of TNT-208 can be generated using knock out, knock-down technology (e.g., miFuc technology), or expression in the presence of a 2-fluorofucose compound in transient 293 mammalian cell line. The non-fucosylated TNT-208 antibody was purified using standard Protein A purification method and conjugated to CpG 7-7 following the same one-step conjugation method using mTG (microbial transglutaminase) enzyme described in Example 10. The non-fucosylated TNT-208 natibody conjugated to CpG 7-7 is designated TNT-363a whereas the fucosylated antibody (TNT-208) conjugated to CpG 7-7 is designated TNT-266a.

DLD-1 was transduced to overexpress nectin-4 and GFP and cultured according to ATCC protocols. Plateletpheresis Leukoreduction Chamber (LRS chamber) was received from Vitalant and diluted 1:3 with PBS. Diluted blood was underlaid with 12 mL Ficoll-Paque. Tubes were centrifuged for 30 minutes at 400×g. PBMCs were collected from the interface, resuspended and washed in FACS buffer (PBS with 0.5% BSA). After one wash, PBMCs were resuspended in Complete RPMI1640 (RPMI1640+10% FBS). Tumor cells (0.025E6/well) were labeled with CellTrace CFSE (ThermoFisher) and plated onto a 96-well format in complete RPMI media. Three-fold serial dilutions of TNT-266a and TNT-263a from 300 nM to 0.41 nM were added to the target cells and incubated at 37° C. for 20 minutes. Freshly isolated PBMCs (0.5 e6/well) in Complete RPMI were added to the wells at 20:1 (effector to target) and incubated at 37° C. under 5% CO2 for 18 hours. Cells were pelleted by centrifugation for five minutes at 400×g and stained at 4° C. in Fixable Viability Dye eFluor 780 (ThermoFisher) diluted 1:4000 in PBS. Cells were washed 1× with FACs buffer and centrifuged and stained at 4° C. in FACS buffer for 30 minutes containing FcR Blocking Reagent (Miltenyi Biotec), anti-CD14, anti-CD11c, anti-CD3, anti-CD19, anti-CD56, anti-CD16, anti-HLADR, anti-CD69, anti-CD86 (ThermoFisher, Biolegend). Cells were centrifuged and washed twice in FACS buffer and fixed in 0.5% paraformaldehyde. Cells were analyzed on Attune NxT Flow Cytometer (ThermoFisher), with subsequent data analysis by Flowjo 10.7 (BD). Dead cells were excluded by gating on the eFluor 780-negative population. T cells were identified as CD3⁺ cells, Monocytes were identified as CD3⁻CD19⁻CD14⁺ cells, NK cells were identified as CD3⁻CD19-CD56⁺ cells and dendritic cells (DC) were identified as CD3⁻CD19⁻CD14⁻CD56-CD11C⁺ HLADR⁺ cells. T cell, monocyte, NK cell, and DC activation levels were assessed by median fluorescent intensity.

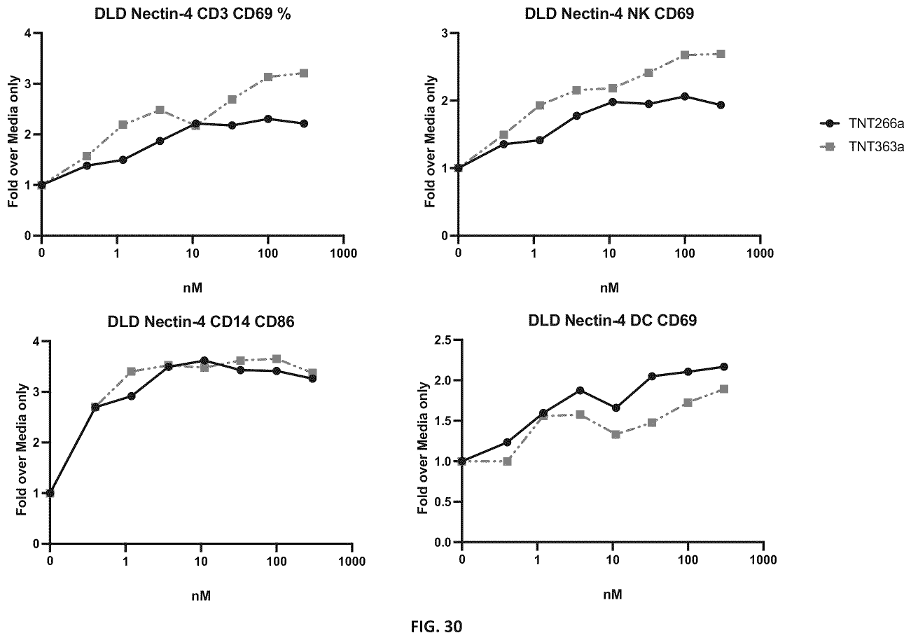

Results are shown in FIG. 30. In the presence of DLD-1 overexpressing nectin-4, non-fucosylated nectin-4 CpG conjugated antibody (TNT-363a) had slightly increased activation of T cells and NK cells compared to non-fucosylated nectin-4 CpG conjugated antibody antibody (TNT-266a) whereas comparable activation was observed for monocytes. Slightly enhanced activation of DCs was observed for TNT-266a compared with TNT-363a.

### Example 26: B Cell Activation by Nectin-4 Antibodies

HT1376, OE19 and H292 cells expressing high to medium to low nectin-4, respectively, were obtained from ATCC and cultured according to their protocols. DLD-1 was transduced to overexpress nectin-4 and GFP and cultured according to ATCC protocols. Plateletpheresis Leukoreduction Chamber (LRS chamber) was received from Vitalant and diluted 1:3 with PBS. Diluted blood was underlaid with 12 mL Ficoll-Paque. Tubes were centrifuged for 30 minutes at 400×g. PBMCs were collected from the interface, resuspended and washed in FACS buffer (PBS with 0.5% BSA). After one wash, PBMCs were resuspended in Complete RPMI1640 (RPMI1640+10% FBS). Tumor cells (0.025E6/well) were labeled with CellTrace CFSE (ThermoFisher) and plated onto a 96-well format in complete RPMI media. Three-fold serial dilutions of nectin-4 CpG-conjugated antibodies from 300 nM to 0.41 nM were added to the target cells and incubated at 37° C. for 20 minutes. Freshly isolated PBMCs (0.5 e6/well) in Complete RPMI were added to the wells at 20:1 (effector to target) and incubated at 37° C. under 5% CO2 for 18 hours. Cells were pelleted by centrifugation for five minutes at 400×g and stained at 4° C. in Fixable Viability Dye eFluor 780 (ThermoFisher) diluted 1:4000 in PBS. Cells were washed 1× with FACs buffer and centrifuged and stained at 4° C. in FACS buffer for 30 minutes containing FcR Blocking Reagent (Miltenyi Biotec), anti-CD19 and anti-CD69 (ThermoFisher, Biolegend). Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (BD). Dead cells were excluded by gating on the eFluor 780-negative population. B cells were identified as CD19′ cells and B cell activation levels were assessed by median fluorescent intensity.

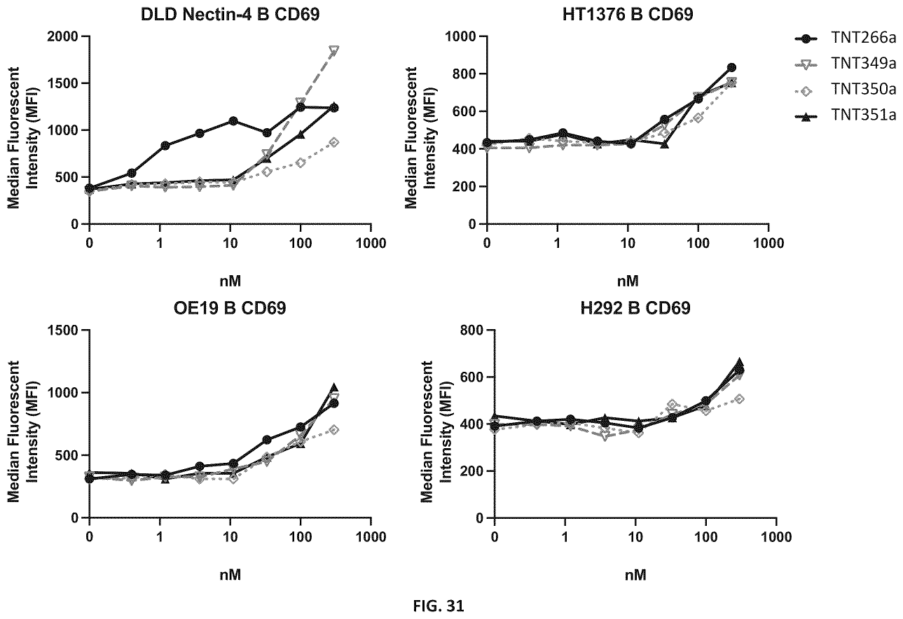

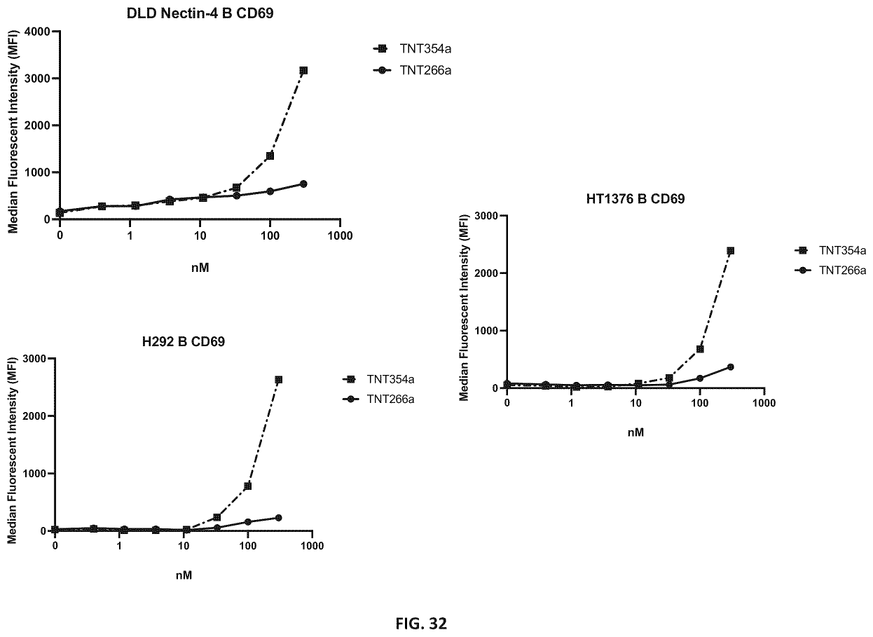

Results are shown in FIGS. 31-32. All nectin-4 CpG conjugated antibodies displayed B cell activation in comparison to media alone. TNT-266a had higher levels of activation in comparison to TNT-329a, TNT-350a, and TNT-351a in DLD-1 expressing nectin-4 (FIG. 31). Interestingly, TNT-354a had significantly enhanced levels of B cell activation in comparison to TNT-266a in DLD-1 cells expressing nectin-4 and endogenous cell lines (H292 and HT1376) (FIG. 32). The observed activation of B-cell is likely occurring through bystander and secondary cascade activation.

### Example 27: Memory Response of Nectin-4 Antibody Conjugates

MC38 tumor cells expressing mouse nectin-4 and GFP (MC38 nectin-4) were generated by retroviral transduction and nectin-4 expressing cells were sorted and expanded. MC38 nectin-4 mouse colon carcinoma cells were cultured in Complete DMEM (DMEM+Glutamax+10% FBS (Gibco)) at 37° C. and 5% CO2. Cells were detached with Trypsin 0.25% (Gibco) and washed twice with DMEM (Gibco). Cells were resuspended at 20E6/mL in DMEM and kept on ice until use. 100 μL of suspended cells were subcutaneously implanted into the right flank of 6-week-old female C57BL/6 mice (Charles River). Tumor size was measured and recorded twice a week with calipers starting 4 days post implantation until duration of the study. Tumor volume was calculated using the following formula: (length×width×width)/2. Once tumors reached an average of 60 mm³, approximately 4 days post implantation, mice were randomized by tumor size and treatments were initiated. TNT-303a and TNT-304a were tested. The conjugates were administered intraperitoneally 3 doses 3 days apart at 3 mg/kg. 100 days post initial tumor clearance, 100 μL of suspended MC38 nectin-4 cells (20E6/mL) were subcutaneously implanted into the flank of tumor free C57BL/6 mice and age-matched control mice (Charles River). Mice whose tumors exceeded 2,000 mm³ or exhibited any signs of distress at any time during the study were euthanized humanely as per IACUC-approved animal protocols.

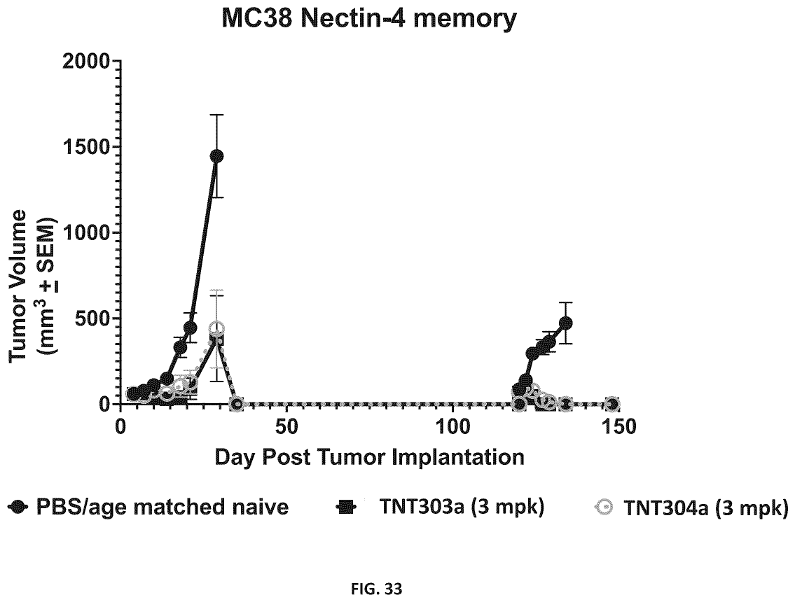

Results are shown in FIG. 33. Both conjugates tested, TNT-303a and TNT-304a, led to durable anti-tumor memory response in MC38 nectin-4 eradicated mice.

### Example 28: Anti-Tumor Activity of Conjugates

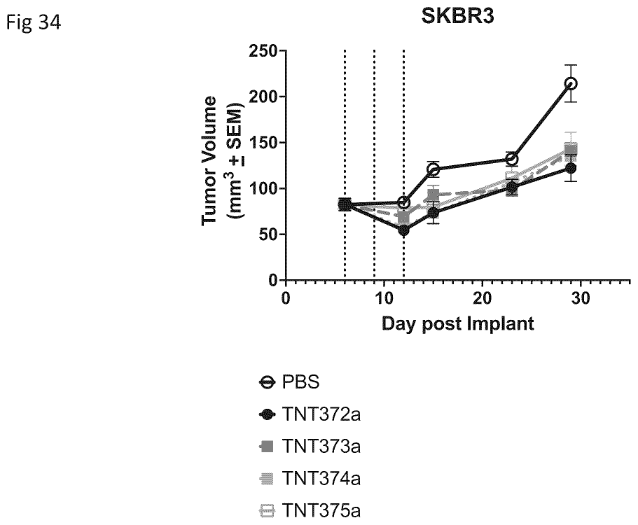

SKBR3 adenocarcinoma cells were cultured in Complete RPMI-1640 (RPMI-1640+Glutamax+10% FBS (Gibco)) at 37° C. and 5% CO2. Cells were detached with Trypsin 0.25% (Gibco) and washed twice with RPMI-1640 (Gibco). After the final wash, cells were resuspended in 1:1 matrigel (Corning): RPMI-1640 at 50E6/mL and kept on ice until use. 100 μL of suspended cells were subcutaneously implanted into the right flank of immunocompromised 6-week-old female nu/nu mice (Charles River). Tumor size was measured and recorded twice a week with calipers starting 6 days post implantation until duration of the study. Tumor volume was calculated using the following formula: (length×width×width)/2. Once tumors reached an average of 86 mm³, approximately 6 days post implantation, mice were randomized by tumor size and treatments were initiated. Nectin-4-antibody-conjugates as human IgG1 (TNT-372a, TNT-373a, TNT-374a and TNT-375a) were administered intraperitoneally 3 doses 3 days apart at 3 mg/kg. Mice whose tumors exceeded 2,000 mm³ or exhibited any signs of distress at any time during the study were euthanized humanely as per IACUC-approved animal protocols. Results are shown in FIG. 34. The systemic administration of the Nectin-4-antibody conjugates exhibited single agent activity in the immunocompromised SKBR3 mouse model.

### Example 29: Anti-tumor activity in checkpoint inhibitor refractory model

CT26 tumor cells expressing mouse nectin-4 (CT26 nectin-4) were generated by retroviral transduction and medium and low nectin-4 expressing cells were sorted and expanded. EMT6 tumor cells expressing mouse nectin-4 (EMT6 nectin-4) were generated by retroviral transduction and medium and low nectin-4 expressing cells were sorted and expanded. CT26 nectin-4 mouse colon carcinoma cells and EMT6 nectin-4 mouse mammary carcinoma cells were cultured in Complete RPMI-1640 (RPMI-1640+Glutamax+10% FBS (Gibco)) at 37° C. and 5% CO2. Cells were detached with Trypsin 0.25% (Gibco) and washed twice with RPMI-1640 (Gibco). Cells were resuspended at 20E6/mL in RPMI-1640 and kept on ice until use. 100 μL of suspended cells were subcutaneously implanted into the right flank of 6-week-old female Balb/c mice (Charles River). Tumor size was measured and recorded twice a week with calipers starting 5 days post implantation for EMT6 nectin-4 and 7 days post implantation for CT26 nectin-4. Tumor volume was calculated using the following formula: (length×width×width)/2. For medium and low EMT6 nectin-4, once tumors reached an average of 75 mm³, approximately 5 days post implantation, mice were randomized by tumor size and treatments were initiated. For medium and low CT26 nectin-4, once tumors reached an average of 84 mm³ and 73 mm³ respectively, mice were randomized by tumor size and treatments were initiated. The conjugates were administered intraperitoneally 3 doses 3 days apart at 3 mg/kg. Mice whose tumors exceeded 2,000 mm³ or exhibited any signs of distress at any time during the study were euthanized humanely as per IACUC-approved animal protocols.

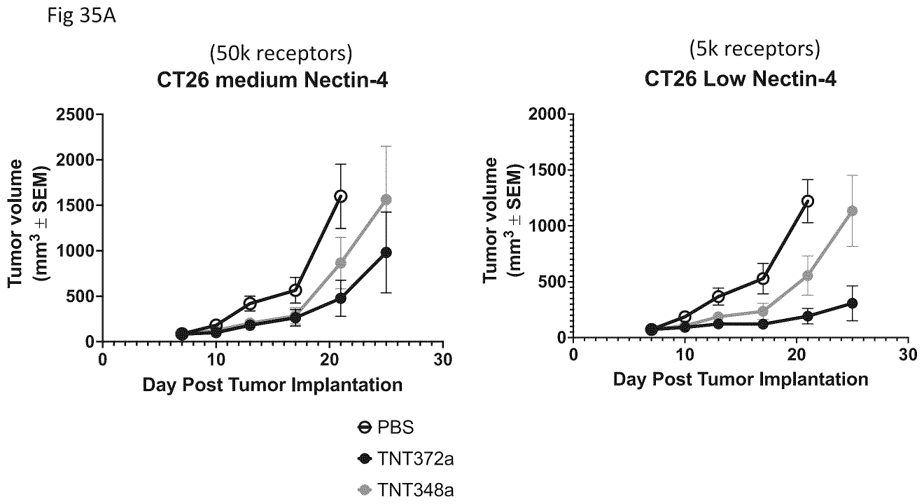

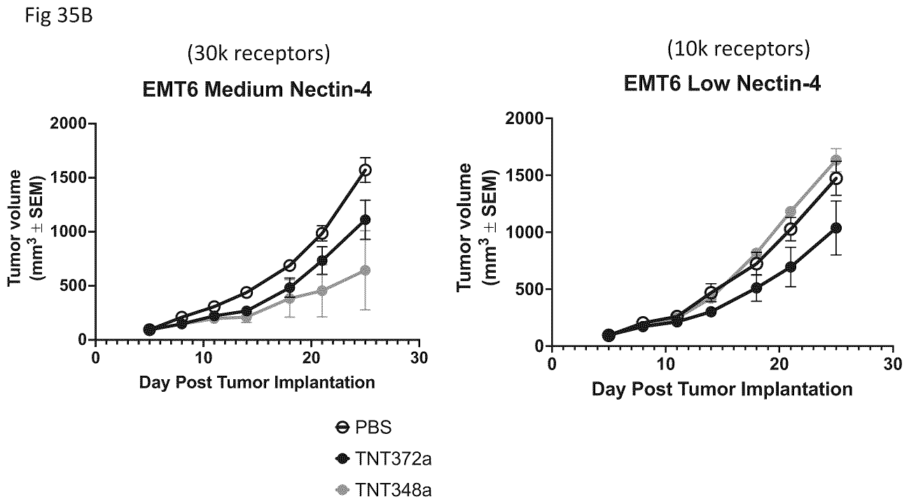

Systemic administration of the conjugates exhibited single agent activity in both medium and low expressing CT26 and EMT6 Nectin-4 mouse models. Results are shown in FIG. 35A (CT26 model) and FIG. 35B (EMT6 model; checkpoint inhibitor refractory model).

### Example 30: Anti-Tumor Activity with an Enhanced Fc Domain

CT26 tumor cells expressing mouse nectin-4 (CT26 nectin-4) were generated by retroviral transduction and medium and low nectin-4 expressing cells were sorted and expanded. EMT6 tumor cells expressing mouse nectin-4 (EMT6 nectin-4) were generated by retroviral transduction and medium and low nectin-4 expressing cells were sorted and expanded. CT26 nectin-4 mouse colon carcinoma cells and EMT6 nectin-4 mouse mammary carcinoma cells were cultured in Complete RPMI-1640 (RPMI-1640+Glutamax+10% FBS (Gibco)) at 37° C. and 5% CO2. Cells were detached with Trypsin 0.25% (Gibco) and washed twice with RPMI-1640 (Gibco). Cells were resuspended at 20E6/mL in RPMI-1640 and kept on ice until use. 100 μL of suspended cells were subcutaneously implanted into the right flank of 6-week-old female Balb/c mice (Charles River). Tumor size was measured and recorded twice a week with calipers starting 5 days post implantation for EMT6 nectin-4 and 7 days post implantation for CT26 Nectin-4. Tumor volume was calculated using the following formula: (length×width×width)/2. For medium and low EMT6 Nectin-4, once tumors reached an average of 75 mm³, approximately 5 days post implantation, mice were randomized by tumor size and treatments were initiated. For medium and low CT26 Nectin-4, once tumors reached an average of 84 mm³ and 73 mm³ respectively, mice were randomized by tumor size and treatments were initiated. TNT348a and TNT303a were administered intraperitoneally 3 doses 3 days apart at 3 mg/kg. Mice whose tumors exceeded 2,000 mm³ or exhibited any signs of distress at any time during the study were euthanized humanely as per IACUC-approved animal protocols.

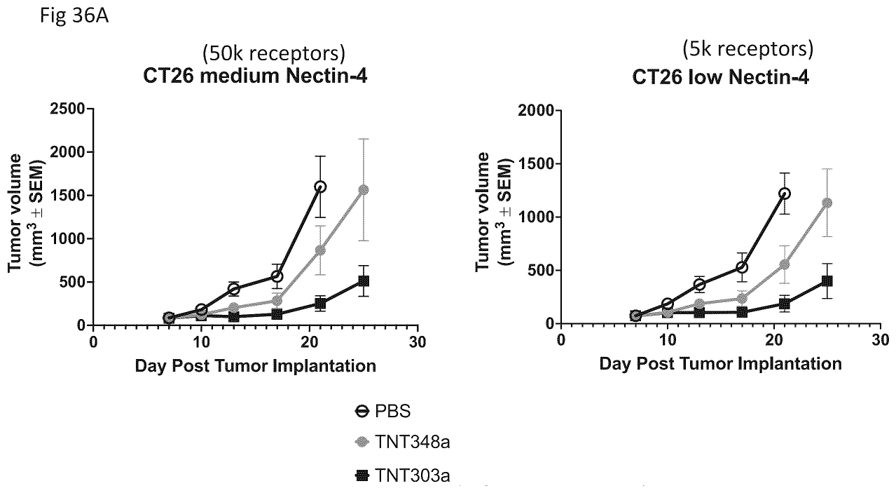

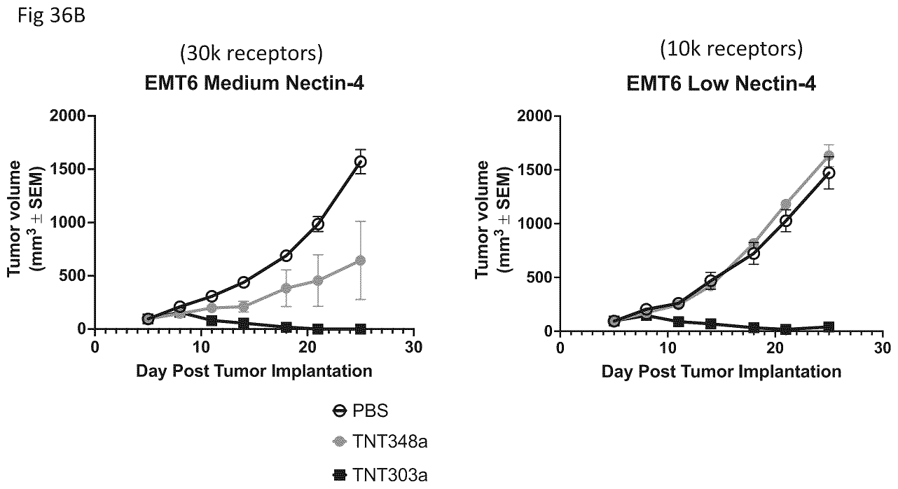

Results are shown in FIG. 36A (CT26 model) and FIG. 36B (EMT6; checkpoint inhibitor refractory model). The conjugate with the enhanced Fc domain (having higher affinity to mouse FcγRs) (TNT-303a) exhibited potent and higher activity as compared to the conjugate with an Fc domain having reduced affinity to mouse FcγRs (TNT-348a). For TNT-303a, tumor eradication was observed at 3 mg/kg for 5 out of 5 mice and 2 out of 5 mice in the CPI refractory medium EMT6 Nectin-4 and low EMT6 Nectin-4 tumor models, respectively.

### Example 31: DAR Comparison

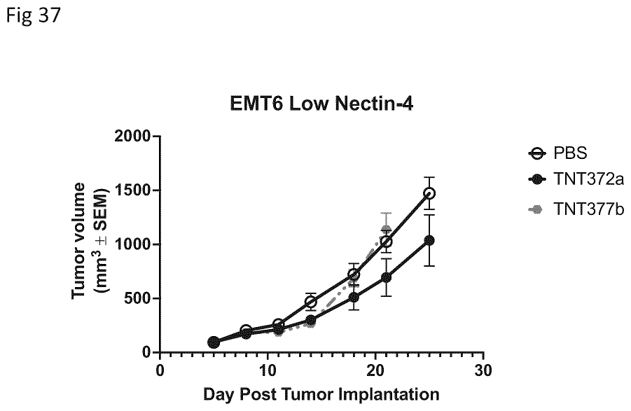

EMT6 tumor cells expressing mouse nectin-4 (EMT6 nectin-4) were generated by retroviral transduction and low nectin-4 expressing cells were sorted and expanded. EMT6 nectin-4 mouse mammary carcinoma cells were cultured in Complete RPMI-1640 (RPMI-1640+Glutamax+10% FBS (Gibco)) at 37° C. and 5% CO2. Cells were detached with Trypsin 0.25% (Gibco) and washed twice with RPMI-1640 (Gibco). Cells were resuspended at 20E6/mL in RPMI-1640 and kept on ice until use. 100 μL of suspended cells were subcutaneously implanted into the right flank of 6-week-old female Balb/C mice (Charles River). Tumor size was measured and recorded twice a week with calipers starting 5 days post implantation for EMT6 nectin-4. Tumor volume was calculated using the following formula: (length×width×width)/2. For low EMT6 nectin-4, once tumors reached an average of 75 mm³, approximately 5 days post implantation, mice were randomized by tumor size and treatments were initiated. Nectin-4 antibody-CpG conjugate with a 1:1 CpG to antibody ratio (TNT-372a) or with a 2:1 CpG conjugation to antibody ratio (TNT-377b) was administered intraperitoneally 3 doses 3 days apart at 3 mg/kg. Mice whose tumors exceeded 2,000 mm³ or exhibited any signs of distress at any time during the study were euthanized humanely as per IACUC-approved animal protocols. Results are shown in FIG. 37. The conjugate with a 2:1 ratio exhibited less efficacy compared to the conjugate with a 1:1 ratio.

### Example 32: Activity of Non-Fucosylated Conjugate in High and Low-Expressing Nectin 4 Cell Lines

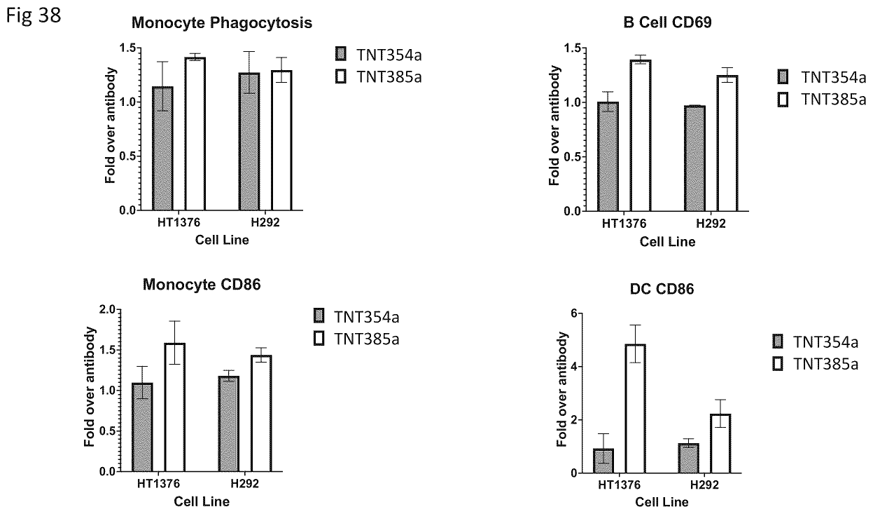

The high nectin-4 expressing bladder cancer cell line HT1376 or low nectin-4 expressing lung cancer cell line H292 was labeled with 300 nM CellTrace CFSE (ThermoFisher) and immediately plated into a 96-well format (0.025E6/well). Leukocyte reduction chambers were received from Vitalant and diluted 1:3 with Phosphate Buffered Saline (PBS, Gibco). Diluted blood was underlaid with 12 mL Ficoll-Paque (GE Healthcare). Tubes were centrifuged for 30 minutes at 400×g. PBMCs were collected from the interface, resuspended and washed in FACS buffer (PBS with 0.5% Bovine Serum Albumin, Gibco). After one wash, PBMCs were resuspended in Complete RPMI-1640 (RPMI-1640+10% FBS, Gibco). PBMCs were immediately added to plated tumor cells at 20:1 (effector to target). 300 nM of non-fucosylated nectin-4 CpG conjugate (TNT-385a) or fucosylated nectin-4 CpG conjugate (TNT-354a) or their respective unconjugated antibody controls were added to wells at 37° C. under 5% CO2 for 18 hours. Cells were dissociated with TrypLE (ThermoFisher), washed and pelleted by centrifugation for five minutes at 400×g before staining for 60 minutes at 4° C. in Fixable Viability Dye eFluor 780 (Thermo Fisher) diluted 1:5000 in PBS. Cells were washed and centrifuged and stained for 60 minutes at 4° C. in FACS buffer containing FcR Blocking Reagent (Miltenyi Biotec), anti-CD14, anti-CD11c, anti-CD3, anti-CD20, anti-CD56, anti-CD16, anti-HLADR, anti-CD69 and anti-CD86 (ThermoFisher, Biolegend). Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (BD). Dead cells were excluded by gating on the eFluor 780-negative population. B cells were identified as CD20⁺ cells. Monocytes were identified as CD20⁻CD3⁻CD14⁺ cells. DCs were identified as CD3⁻CD19⁻CD14⁻CD56-HLADR⁺CD11c⁺ cells. Activation levels were assessed by median fluorescent intensity (MFI) and phagocytosis was measured as % CFSE positive monocyte cells. Results are shown in FIG. 38; Activation and phagocytosis data is graphed as fold activity of the nectin-4 CpG conjugate over the respective nectin-4 antibody (unconjugated). The non-fucosylated nectin-4 CpG conjugate (TNT-385a) exhibited higher activation (B cells, monocytes, and DCs) and phagocytosis as compared to the corresponding fucosylated nectin-4 CpG conjugate (TNT-354a) upon engagement of both high and low nectin-4 positive tumor cells.

### Example 33: Activity of Fe Variant in High and Low-Expressing Nectin 4 Cell Lines

High nectin-4 expressing bladder cancer cell line HT1376 and low nectin-4 expressing lung cancer cell line H292 were labeled with 300 nM CellTrace CFSE (ThermoFisher) and immediately plated into a 96-well format (0.025E6/well). Leukocyte reduction chambers were received from Vitalant and diluted 1:3 with Phosphate Buffered Saline (PBS, Gibco). Diluted blood was underlaid with 12 mL Ficoll-Paque (GE Healthcare). Tubes were centrifuged for 30 minutes at 400×g. PBMCs were collected from the interface, resuspended and washed in FACS buffer (PBS with 0.5% Bovine Serum Albumin, Gibco). After one wash, PBMCs were resuspended in Complete RPMI-1640 (RPMI-1640+10% FBS, Gibco). PBMCs were immediately added to plated tumor cells at 20:1 (effector to target). Four-fold serial dilutions were added to the cells from 1 uM to 0.2 nM of fucosylated human IgG1 nectin-4 CpG conjugate (TNT-415a), fucosylated human IgG1 G236A mutated (TNT-416a), or their respective non-fucosylated nectin-4 CpG conjugates (TNT-415a-AF, TNT-416a-AF) along with their respective antibody alone controls. After 18 hour incubation at 37° C. under 5% CO2, cells were dissociated with TrypLE (ThermoFisher), washed and pelleted by centrifugation for five minutes at 400×g before staining for 30 minutes at 4° C. in Fixable Viability Dye eFluor 780 (Thermo Fisher) diluted 1:5000 in PBS. Cells were washed and centrifuged and stained for 60 minutes at 4° C. in FACS buffer containing FcR Blocking Reagent (Miltenyi Biotec), anti-CD14, anti-CD11c, anti-CD3, anti-CD20, anti-CD56, anti-CD16, anti-HLADR, anti-CD69 and anti-CD86 (ThermoFisher, Biolegend). Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (BD). Dead cells were excluded by gating on the eFluor 780-negative population. B cells were identified as CD20⁺ cells. Monocytes were identified as CD20⁻CD3⁻CD14⁺ cells. DCs were identified as CD3⁻CD19⁻CD14⁻CD56-HLADR⁺CD11c⁺ cells. Cell counts were determined from events collected within set volume of acquisition.

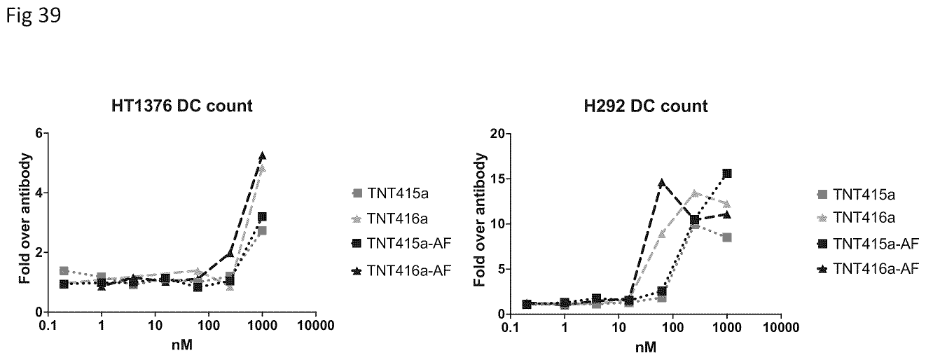

Dendritic cell (DC) proliferation was observed with all conjugates. The G236A mutated Fc Nectin-4 CpG conjugate (fucosylated and non-fucosylated) resulted in higher levels of DC proliferation as compared to the respective nonmutated Fc conjugates. No difference in activity was observed in monocytes, NK cells, T cells, and B cells. Results for the DC proliferation in HT1376 and H292 are shown in FIG. 39 as fold activity of nectin-4 CpG conjugate fold over the respective nectin-4 antibody control.

### Example 34: Tumor-Killing Activity

Low nectin-4 expressing lung cancer cell line H292 and very low nectin-4 expressing prostate cancer cell line PC3 were labeled with 300 nM CellTrace CFSE (ThermoFisher) and immediately plated into a 96-well format (0.025E6/well). Leukocyte reduction chambers were received from Vitalant and diluted 1:3 with Phosphate Buffered Saline (PBS, Gibco). Diluted blood was underlaid with 12 mL Ficoll-Paque (GE Healthcare). Tubes were centrifuged for 30 minutes at 400×g. PBMCs were collected from the interface, resuspended and washed in FACS buffer (PBS with 0.5% Bovine Serum Albumin, Gibco). After one wash, PBMCs were resuspended in Complete RPMI-1640 (RPMI-1640+10% FBS, Gibco). PBMCs were immediately added to plated tumor cells at 20:1 (effector to target). Four-fold serial dilutions were added to the cells from 1 μM to 0.2 nM of fucosylated human IgG1 nectin-4 CpG conjugate (TNT-415a), fucosylated human IgG1 G236A mutated (TNT-416a), their respective non-fucosylated nectin-4 CpG conjugates (TNT-415a-AF and TNT-416a-AF) along with their respective antibody-alone (not conjugated to CpG) controls. After 18 hour incubation at 37° C. under 5% CO2, cells were dissociated with TrypLE (ThermoFisher), washed and pelleted by centrifugation for five minutes at 400×g before staining for 30 minutes at 4° C. in Fixable Viability Dye eFluor 780 (Thermo Fisher) diluted 1:5000 in PBS. Cells were washed and centrifuged and stained for 60 minutes at 4° C. in FACS buffer containing FcR Blocking Reagent (Miltenyi Biotec), anti-CD14, anti-CD11c, anti-CD3, anti-CD20, anti-CD56, anti-CD16, anti-HLADR, anti-CD69 and anti-CD86 (ThermoFisher, Biolegend). Cells were centrifuged and washed twice in FACS buffer and fixed in 0.5% paraformaldehyde. Cells were acquired within a set acquisition volume on Attune NxT Flow Cytometer (Thermo Fisher), with subsequent data analysis by Flowjo 10.7 (BD). Target cancer cells were identified by CFSE+, FFC-high and SSC-high population. From target cell population, dead cells were excluded by gating on the eFluor 780-negative population and identified as % live of target cancer cell. Data was plotted as nectin-4 CpG conjugates fold over their respective nectin-4 antibody control.

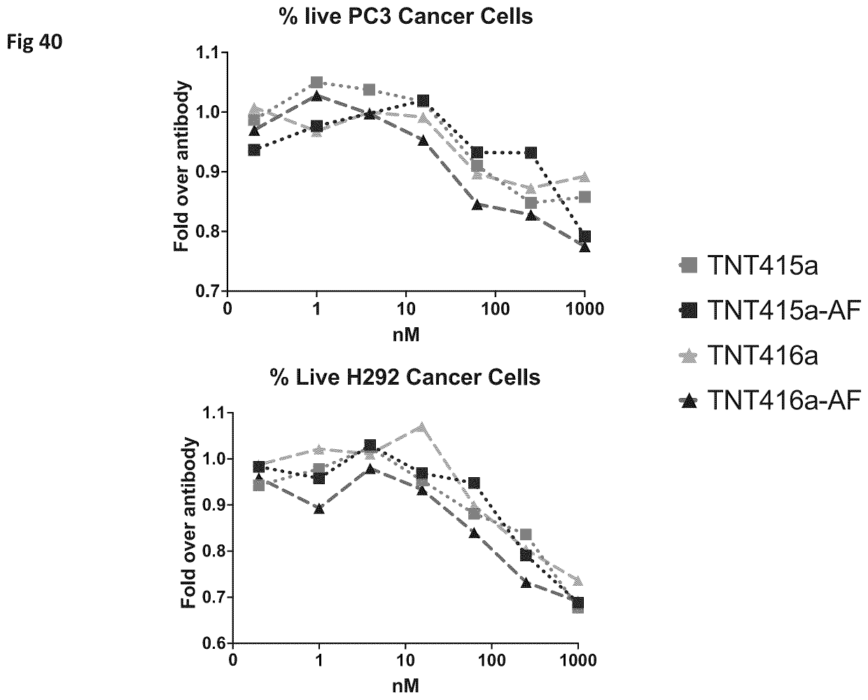

Tumor cell cytoxicity was observed with all conjugates. Results for cancer cell viability are shown in FIG. 40 as fold activity of nectin-4 CpG conjugate fold over the respective nectin-4 antibody control.

1. A conjugate comprising (i) an anti-nectin-4 antibody or antigen binding fragment thereof and (ii) one or more immunomodulating oligonucleotides (P), wherein the antibody or antigen-binding fragment is linked to one or more Q-tag peptides (Q) comprising at least one glutamine residue, and wherein each immunomodulating oligonucleotide is linked to a Q-tag peptide via an amide bond with the glutamine residue of the Q-tag peptide and a linker (L) as shown in Formula (A): 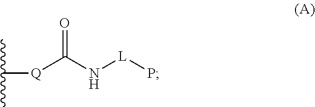
wherein [symbol] indicates the point of attachment of each Q to the antibody or antigen-binding fragment thereof (Ab);
wherein each P is independently an immunomodulating oligonucleotide comprising the structure 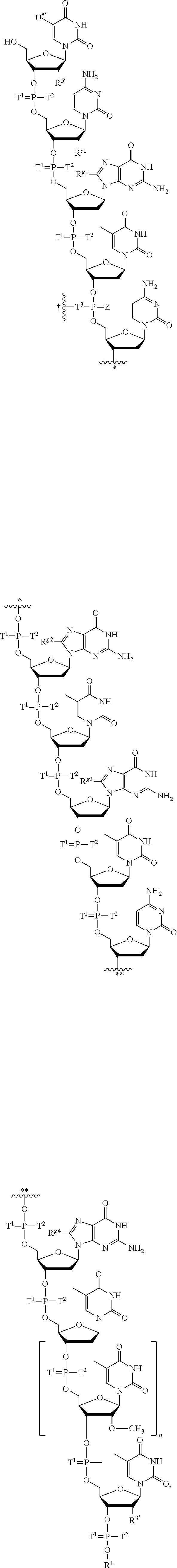
wherein [symbol]
* and [symbol] ** indicate the points of attachment within the oligonucleotide, and wherein [symbol] † indicates the point of attachment to the linker L;
each T¹ is independently O or S;
each T² is S⁻;
T³ is a group 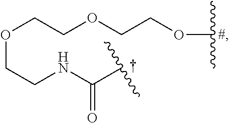
wherein [symbol] † indicates the point of attachment to L and wherein [symbol] # indicates the point of attachment to the rest of the oligonucleotide;
Z is O or S;
U^{5′} is —H or halogen;
R^{5′} is —H or methoxy;
R¹ is —H or methoxy;
R^{g1}, R^{g2}, R^{g3}, and R^{g4} are H;
R^{3′} is methoxy;
R¹ is —(CH₂)₃—OH;
R² is —H or methyl; and
n is an integer from 0 to 2.

2. The conjugate of claim 1, wherein
each P is independently an immunomodulating oligonucleotide having the structure

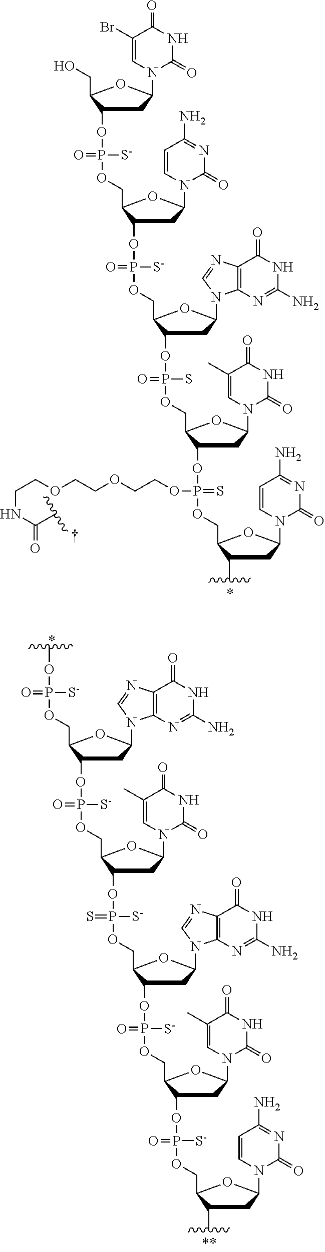

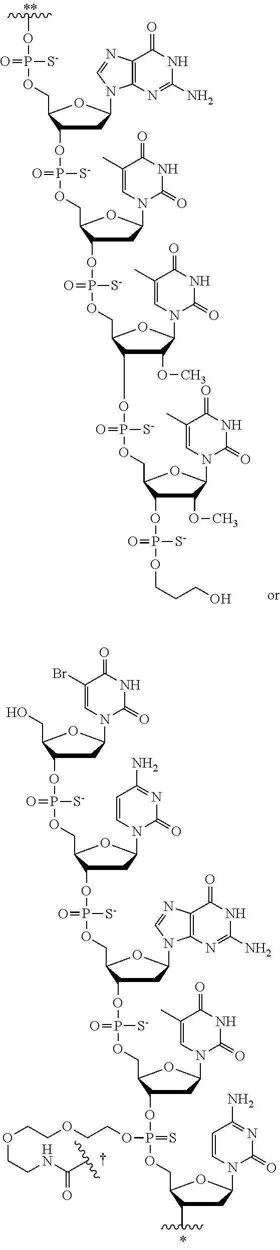

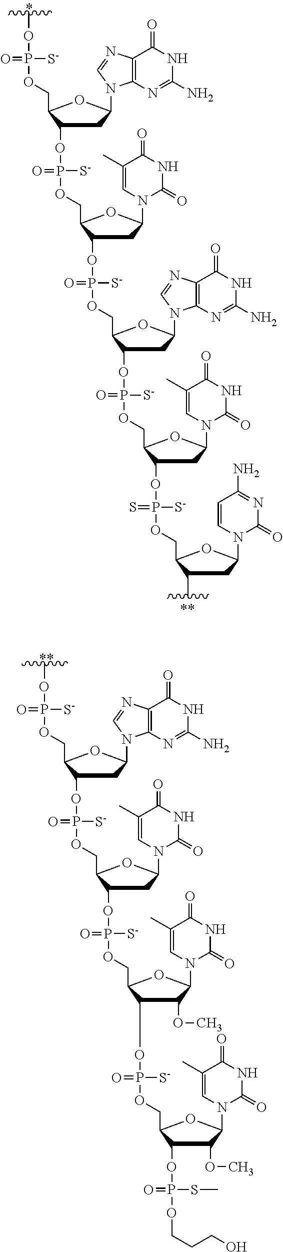

3. (canceled)

4. A conjugate comprising (i) an anti-nectin-4 antibody or antigen binding fragment thereof and (ii) one or more immunomodulating oligonucleotides (P),
wherein the antibody or antigen-binding fragment is linked to one or more Q-tag peptides (Q) that comprise the amino acid sequence RPQGF (SEQ ID NO:47),
and wherein each immunomodulating oligonucleotide (P) is linked to a Q-tag peptide via an amide bond with the glutamine residue of the Q-tag peptide and a linker (L) as shown in Formula (A): 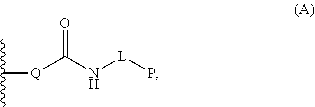
wherein [symbol] indicates the point of attachment of each Q to the antibody or antigen-binding fragment thereof (Ab).

5-6. (canceled)

7. The conjugate of claim 1, wherein the antibody comprises a light chain variable region (VL) domain and a heavy chain variable region (VH) domain, wherein:
(a) the VL domain comprises CDR-L1, CDR-L2, and CDR-L3 sequences from a VL domain selected from the group consisting of SEQ ID NOs:248, 250, 252, 254, 256, 258, 260, 262, 264, 266, 268, 270, 900, 902, 904, 906, 908, 910, 912, 914, 916, 918, 920, 922, 924, 926, 928, 930, 932, 934, 936, 938, 940, 942, and 944 and the VH domain comprises CDR-H1, CDR-H2, and CDR-H3 sequences from a VH domain selected from the group consisting of SEQ ID NOs:249, 251, 253, 255, 257, 259, 261, 263, 265, 267, 269, 271, 901, 903, 905, 907, 909, 911, 913, 915, 917, 919, 921, 923, 925, 927, 929, 931, 933, 935, 937, 939, 941, 943, and 945;
(b) the VL domain comprises CDR-L1, CDR-L2, and CDR-L3 sequences from a VL domain of SEQ ID NO:248 and the VH domain comprises CDR-H1, CDR-H2, and CDR-H3 from a VH domain of SEQ ID NO:249;
(c) the VL domain comprises CDR-L1, CDR-L2, and CDR-L3 sequences from a VL domain of SEQ ID NO:250 and the VH domain comprises CDR-H1, CDR-H2, and CDR-H3 from a VH domain of SEQ ID NO:251;
(d) the VL domain comprises CDR-L1, CDR-L2, and CDR-L3 sequences from a VL domain of SEQ ID NO:252 and the VH domain comprises CDR-H1, CDR-H2, and CDR-H3 from a VH domain of SEQ ID NO:253;
(e) the VL domain comprises CDR-L1, CDR-L2, and CDR-L3 sequences from a VL domain of SEQ ID NO:254 and the VH domain comprises CDR-H1, CDR-H2, and CDR-H3 from a VH domain of SEQ ID NO:255;
(f) the VL domain comprises CDR-L1, CDR-L2, and CDR-L3 sequences from a VL domain of SEQ ID NO:256 and the VH domain comprises CDR-H1, CDR-H2, and CDR-H3 from a VH domain of SEQ ID NO:257;
(g) the VL domain comprises CDR-L1, CDR-L2, and CDR-L3 sequences from a VL domain of SEQ ID NO:258 and the VH domain comprises CDR-H1, CDR-H2, and CDR-H3 from a VH domain of SEQ ID NO:259;
(h) the VL domain comprises CDR-L1, CDR-L2, and CDR-L3 sequences from a VL domain of SEQ ID NO:260 and the VH domain comprises CDR-H1, CDR-H2, and CDR-H3 from a VH domain of SEQ ID NO:261;
(i) the VL domain comprises CDR-L1, CDR-L2, and CDR-L3 sequences from a VL domain of SEQ ID NO:262 and the VH domain comprises CDR-H1, CDR-H2, and CDR-H3 from a VH domain of SEQ ID NO:263;
(j) the VL domain comprises CDR-L1, CDR-L2, and CDR-L3 sequences from a VL domain of SEQ ID NO:264 and the VH domain comprises CDR-H1, CDR-H2, and CDR-H3 from a VH domain of SEQ ID NO:265;
(k) the VL domain comprises CDR-L1, CDR-L2, and CDR-L3 sequences from a VL domain of SEQ ID NO:266 and the VH domain comprises CDR-H1, CDR-H2, and CDR-H3 from a VH domain of SEQ ID NO:267;
(1) the VL domain comprises CDR-L1, CDR-L2, and CDR-L3 sequences from a VL domain of SEQ ID NO:268 and the VH domain comprises CDR-H1, CDR-H2, and CDR-H3 from a VH domain of SEQ ID NO:269;
(m) the VL domain comprises CDR-L1, CDR-L2, and CDR-L3 sequences from a VL domain of SEQ ID NO:270 and the VH domain comprises CDR-H1, CDR-H2, and CDR-H3 from a VH domain of SEQ ID NO:271;
(n) the VL domain comprises CDR-L1, CDR-L2, and CDR-L3 sequences from a VL domain of SEQ ID NO:900 and the VH domain comprises CDR-H1, CDR-H2, and CDR-H3 from a VH domain of SEQ ID NO:901;
(o) the VL domain comprises CDR-L1, CDR-L2, and CDR-L3 sequences from a VL domain of SEQ ID NO:902 and the VH domain comprises CDR-H1, CDR-H2, and CDR-H3 from a VH domain of SEQ ID NO:903;
(p) the VL domain comprises CDR-L1, CDR-L2, and CDR-L3 sequences from a VL domain of SEQ ID NO:904 and the VH domain comprises CDR-H1, CDR-H2, and CDR-H3 from a VH domain of SEQ ID NO:905;
(q) the VL domain comprises CDR-L1, CDR-L2, and CDR-L3 sequences from a VL domain of SEQ ID NO:906 and the VH domain comprises CDR-H1, CDR-H2, and CDR-H3 from a VH domain of SEQ ID NO:907;
(r) the VL domain comprises CDR-L1, CDR-L2, and CDR-L3 sequences from a VL domain of SEQ ID NO:908 and the VH domain comprises CDR-H1, CDR-H2, and CDR-H3 from a VH domain of SEQ ID NO:909;
(s) the VL domain comprises CDR-L1, CDR-L2, and CDR-L3 sequences from a VL domain of SEQ ID NO:910 and the VH domain comprises CDR-H1, CDR-H2, and CDR-H3 from a VH domain of SEQ ID NO:911;
(t) the VL domain comprises CDR-L1, CDR-L2, and CDR-L3 sequences from a VL domain of SEQ ID NO:912 and the VH domain comprises CDR-H1, CDR-H2, and CDR-H3 from a VH domain of SEQ ID NO:913;
(u) the VL domain comprises CDR-L1, CDR-L2, and CDR-L3 sequences from a VL domain of SEQ ID NO:914 and the VH domain comprises CDR-H1, CDR-H2, and CDR-H3 from a VH domain of SEQ ID NO:915;
(v) the VL domain comprises CDR-L1, CDR-L2, and CDR-L3 sequences from a VL domain of SEQ ID NO:916 and the VH domain comprises CDR-H1, CDR-H2, and CDR-H3 from a VH domain of SEQ ID NO:917;
(w) the VL domain comprises CDR-L1, CDR-L2, and CDR-L3 sequences from a VL domain of SEQ ID NO:918 and the VH domain comprises CDR-H1, CDR-H2, and CDR-H3 from a VH domain of SEQ ID NO:919;
(x) the VL domain comprises CDR-L1, CDR-L2, and CDR-L3 sequences from a VL domain of SEQ ID NO:920 and the VH domain comprises CDR-H1, CDR-H2, and CDR-H3 from a VH domain of SEQ ID NO:921;
(y) the VL domain comprises CDR-L1, CDR-L2, and CDR-L3 sequences from a VL domain of SEQ ID NO:922 and the VH domain comprises CDR-H1, CDR-H2, and CDR-H3 from a VH domain of SEQ ID NO:923;
(z) the VL domain comprises CDR-L1, CDR-L2, and CDR-L3 sequences from a VL domain of SEQ ID NO:924 and the VH domain comprises CDR-H1, CDR-H2, and CDR-H3 from a VH domain of SEQ ID NO:925;
(aa) the VL domain comprises CDR-L1, CDR-L2, and CDR-L3 sequences from a VL domain of SEQ ID NO:926 and the VH domain comprises CDR-H1, CDR-H2, and CDR-H3 from a VH domain of SEQ ID NO:927;
(bb) the VL domain comprises CDR-L1, CDR-L2, and CDR-L3 sequences from a VL domain of SEQ ID NO:928 and the VH domain comprises CDR-H1, CDR-H2, and CDR-H3 from a VH domain of SEQ ID NO:929;
(cc) the VL domain comprises CDR-L1, CDR-L2, and CDR-L3 sequences from a VL domain of SEQ ID NO:930 and the VH domain comprises CDR-H1, CDR-H2, and CDR-H3 from a VH domain of SEQ ID NO:931;
(dd) the VL domain comprises CDR-L1, CDR-L2, and CDR-L3 sequences from a VL domain of SEQ ID NO932; and the VH domain comprises CDR-H1, CDR-H2, and CDR-H3 from a VH domain of SEQ ID NO:933;
(ee) the VL domain comprises CDR-L1, CDR-L2, and CDR-L3 sequences from a VL domain of SEQ ID NO:934 and the VH domain comprises CDR-H1, CDR-H2, and CDR-H3 from a VH domain of SEQ ID NO:935;
(ff) the VL domain comprises CDR-L1, CDR-L2, and CDR-L3 sequences from a VL domain of SEQ ID NO:936 and the VH domain comprises CDR-H1, CDR-H2, and CDR-H3 from a VH domain of SEQ ID NO:937;
(gg) the VL domain comprises CDR-L1, CDR-L2, and CDR-L3 sequences from a VL domain of SEQ ID NO:938 and the VH domain comprises CDR-H1, CDR-H2, and CDR-H3 from a VH domain of SEQ ID NO:939;
(hh) the VL domain comprises CDR-L1, CDR-L2, and CDR-L3 sequences from a VL domain of SEQ ID NO:940 and the VH domain comprises CDR-H1, CDR-H2, and CDR-H3 from a VH domain of SEQ ID NO:941;
(ii) the VL domain comprises CDR-L1, CDR-L2, and CDR-L3 sequences from a VL domain of SEQ ID NO:942 and the VH domain comprises CDR-H1, CDR-H2, and CDR-H3 from a VH domain of SEQ ID NO:943; or
(jj) the VL domain comprises CDR-L1, CDR-L2, and CDR-L3 sequences from a VL domain of SEQ ID NO:944 and the VH domain comprises CDR-H1, CDR-H2, and CDR-H3 from a VH domain of SEQ ID NO:945.

8-12. (canceled)

13. The conjugate of claim 1, wherein the antibody comprises an Fc region.

14-22. (canceled)

23. The conjugate of claim 13, wherein the Fc region has been engineered to improve effector function, optionally wherein the improved effector function is antibody-dependent cell-mediated cytotoxicity (ADCC) activity, antibody-dependent cellular phagocytosis (ADCP) activity, or complement-dependent cytotoxicity (CDC) activity.

24-37. (canceled)

38. The conjugate of claim 13, wherein the conjugate further comprises an immunomodulating oligonucleotide P attached to the Q295 of the Fc region residue as shown in the following formula 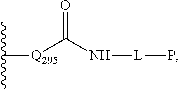
wherein L is a linker moiety connected to Q295 of the Fc region via an amide bond.

39-49. (canceled)

50. The conjugate of a claim 1, wherein
the peptide sequence of each Q-tag is independently selected from the group consisting of SEQ ID NOs: 39-55.

51-54. (canceled)

55. The conjugate of claim 1, wherein the conjugate has a DAR of 1 or 2.

56. (canceled)

57. The conjugate of claim 1, wherein the linker (L) is 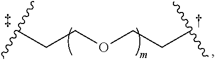
wherein m is an integer ranging from about 0 to about 50, and wherein [symbol] † indicates the point of attachment to T³, and [symbol] ‡ indicates the point of attachment to the rest of the conjugate.

58-59. (canceled)

60. The conjugate of claim 1, wherein Z is S.

61. The conjugate of claim 1, wherein
the oligonucleotide (P) comprises at least one pair or at least two pairs of geminal T¹ and T² wherein T¹ is S and T² is S⁻.

62-89. (canceled)

90. The conjugate of claim 4, wherein each immunomodulating oligonucleotide (P) is independently 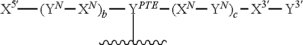
wherein
b and c are each independently an integer from 1 to 25; with the proviso that the sum of b and c is at least 5; [symbol]
* indicates the point of attachment of the immunomodulating oligonucleotide (P) to the rest of the conjugate;
X^{5′} is a 5′ terminal nucleoside comprising the structure 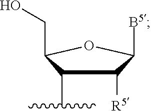
X^{3′} is a 3′ terminal nucleoside comprising the structure 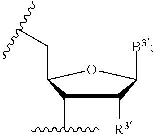
Y^{PTE} is an internucleoside phosphotriester comprising the structure 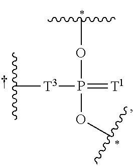
wherein * indicates the points of attachment to the rest of the oligonucleotide and [symbol] † indicates the point of attachment to the linker (L), or, if (L) is absent, [symbol] † indicates the point of attachment to the Q tag peptide at the glutamine residue via an amide bond;
Y^{3′} is a terminal phosphotriester comprising the structure 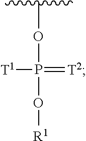
each X^{N} is independently a nucleoside comprising the structure 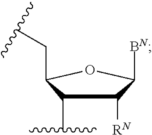
each Y^{N} Is independently an internucleoside linker comprising the structure 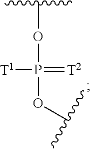
wherein each B^{N} is independently a modified or unmodified nucleobase;
each R^{N} is independently —H or —O—C₁₋₄-alkyl, wherein the C₁₋₄-alkyl of the —O—C₁₋₄-alkyl is optionally further substituted by —O—C₁-C₄-alkyl;
B^{5′} and B^{3′} are independently a modified or unmodified nucleobase;
R^{5′} and R^{3′} are independently —H or —O—C₁-C₄-alkyl, wherein the C₁₋₄-alkyl of the —O—C₁₋₄-alkyl is optionally further substituted by —O—C₁₋₄-alkyl;
each T₁ is independently O or S;
each T₂ is independently O⁻or S⁻; and
T₃ is a group comprising an oligoethylene glycol moiety; and
R¹ is C₁₋₄-alkylene-hydroxy.

91-108. (canceled)

109. An antibody that specifically binds to human nectin-4, wherein the antibody comprises a light chain variable region (VL) domain and a heavy chain variable region (VH) domain, wherein:
(a) the VL domain comprises CDR-L1, CDR-L2, and CDR-L3 sequences from a VL domain selected from the group consisting of SEQ ID NOs:248, 250, 252, 254, 256, 258, 260, 262, 264, 266, 268, 270, 900, 902, 904, 906, 908, 910, 912, 914, 916, 918, 920, 922, 924, 926, 928, 930, 932, 934, 936, 938, 940, 942, and 944 and the VH domain comprises CDR-H1, CDR-H2, and CDR-H3 sequences from a VH domain selected from the group consisting of SEQ ID NOs:249, 251, 253, 255, 257, 259, 261, 263, 265, 267, 269, 271, 901, 903, 905, 907, 909, 911, 913, 915, 917, 919, 921, 923, 925, 927, 929, 931, 933, 935, 937, 939, 941, 943, and 945;
(b) the VL domain comprises CDR-L1, CDR-L2, and CDR-L3 sequences from a VL domain of SEQ ID NO:248 and the VH domain comprises CDR-H1, CDR-H2, and CDR-H3 from a VH domain of SEQ ID NO:249;
(c) the VL domain comprises CDR-L1, CDR-L2, and CDR-L3 sequences from a VL domain of SEQ ID NO:250 and the VH domain comprises CDR-H1, CDR-H2, and CDR-H3 from a VH domain of SEQ ID NO:251;
(d) the VL domain comprises CDR-L1, CDR-L2, and CDR-L3 sequences from a VL domain of SEQ ID NO:252 and the VH domain comprises CDR-H1, CDR-H2, and CDR-H3 from a VH domain of SEQ ID NO:253;
(e) the VL domain comprises CDR-L1, CDR-L2, and CDR-L3 sequences from a VL domain of SEQ ID NO:254 and the VH domain comprises CDR-H1, CDR-H2, and CDR-H3 from a VH domain of SEQ ID NO:255;
(f) the VL domain comprises CDR-L1, CDR-L2, and CDR-L3 sequences from a VL domain of SEQ ID NO:256 and the VH domain comprises CDR-H1, CDR-H2, and CDR-H3 from a VH domain of SEQ ID NO:257;
(g) the VL domain comprises CDR-L1, CDR-L2, and CDR-L3 sequences from a VL domain of SEQ ID NO:258 and the VH domain comprises CDR-H1, CDR-H2, and CDR-H3 from a VH domain of SEQ ID NO:259;
(h) the VL domain comprises CDR-L1, CDR-L2, and CDR-L3 sequences from a VL domain of SEQ ID NO:260 and the VH domain comprises CDR-H1, CDR-H2, and CDR-H3 from a VH domain of SEQ ID NO:261;
(i) the VL domain comprises CDR-L1, CDR-L2, and CDR-L3 sequences from a VL domain of SEQ ID NO:262 and the VH domain comprises CDR-H1, CDR-H2, and CDR-H3 from a VH domain of SEQ ID NO:263;
(j) the VL domain comprises CDR-L1, CDR-L2, and CDR-L3 sequences from a VL domain of SEQ ID NO:264 and the VH domain comprises CDR-H1, CDR-H2, and CDR-H3 from a VH domain of SEQ ID NO:265;
(k) the VL domain comprises CDR-L1, CDR-L2, and CDR-L3 sequences from a VL domain of SEQ ID NO:266 and the VH domain comprises CDR-H1, CDR-H2, and CDR-H3 from a VH domain of SEQ ID NO:267;
(l) the VL domain comprises CDR-L1, CDR-L2, and CDR-L3 sequences from a VL domain of SEQ ID NO:268 and the VH domain comprises CDR-H1, CDR-H2, and CDR-H3 from a VH domain of SEQ ID NO:269;
(m) the VL domain comprises CDR-L1, CDR-L2, and CDR-L3 sequences from a VL domain of SEQ ID NO:270 and the VH domain comprises CDR-H1, CDR-H2, and CDR-H3 from a VH domain of SEQ ID NO:271
(n) the VL domain comprises CDR-L1, CDR-L2, and CDR-L3 sequences from a VL domain of SEQ ID NO:900 and the VH domain comprises CDR-H1, CDR-H2, and CDR-H3 from a VH domain of SEQ ID NO:901;
(o) the VL domain comprises CDR-L1, CDR-L2, and CDR-L3 sequences from a VL domain of SEQ ID NO:902 and the VH domain comprises CDR-H1, CDR-H2, and CDR-H3 from a VH domain of SEQ ID NO:903;
(p) the VL domain comprises CDR-L1, CDR-L2, and CDR-L3 sequences from a VL domain of SEQ ID NO:904 and the VH domain comprises CDR-H1, CDR-H2, and CDR-H3 from a VH domain of SEQ ID NO:905;
(q) the VL domain comprises CDR-L1, CDR-L2, and CDR-L3 sequences from a VL domain of SEQ ID NO:906 and the VH domain comprises CDR-H1, CDR-H2, and CDR-H3 from a VH domain of SEQ ID NO:907;
(r) the VL domain comprises CDR-L1, CDR-L2, and CDR-L3 sequences from a VL domain of SEQ ID NO:908 and the VH domain comprises CDR-H1, CDR-H2, and CDR-H3 from a VH domain of SEQ ID NO:909;
(s) the VL domain comprises CDR-L1, CDR-L2, and CDR-L3 sequences from a VL domain of SEQ ID NO:910 and the VH domain comprises CDR-H1, CDR-H2, and CDR-H3 from a VH domain of SEQ ID NO:911;
(t) the VL domain comprises CDR-L1, CDR-L2, and CDR-L3 sequences from a VL domain of SEQ ID NO:912 and the VH domain comprises CDR-H1, CDR-H2, and CDR-H3 from a VH domain of SEQ ID NO:913;
(u) the VL domain comprises CDR-L1, CDR-L2, and CDR-L3 sequences from a VL domain of SEQ ID NO:914 and the VH domain comprises CDR-H1, CDR-H2, and CDR-H3 from a VH domain of SEQ ID NO:915;
(v) the VL domain comprises CDR-L1, CDR-L2, and CDR-L3 sequences from a VL domain of SEQ ID NO:916 and the VH domain comprises CDR-H1, CDR-H2, and CDR-H3 from a VH domain of SEQ ID NO:917;
(w) the VL domain comprises CDR-L1, CDR-L2, and CDR-L3 sequences from a VL domain of SEQ ID NO:918 and the VH domain comprises CDR-H1, CDR-H2, and CDR-H3 from a VH domain of SEQ ID NO:919;
(x) the VL domain comprises CDR-L1, CDR-L2, and CDR-L3 sequences from a VL domain of SEQ ID NO:920 and the VH domain comprises CDR-H1, CDR-H2, and CDR-H3 from a VH domain of SEQ ID NO:921;
(y) the VL domain comprises CDR-L1, CDR-L2, and CDR-L3 sequences from a VL domain of SEQ ID NO:922 and the VH domain comprises CDR-H1, CDR-H2, and CDR-H3 from a VH domain of SEQ ID NO:923;
(z) the VL domain comprises CDR-L1, CDR-L2, and CDR-L3 sequences from a VL domain of SEQ ID NO:924 and the VH domain comprises CDR-H1, CDR-H2, and CDR-H3 from a VH domain of SEQ ID NO:925;
(aa) the VL domain comprises CDR-L1, CDR-L2, and CDR-L3 sequences from a VL domain of SEQ ID NO:926 and the VH domain comprises CDR-H1, CDR-H2, and CDR-H3 from a VH domain of SEQ ID NO:927;
(bb) the VL domain comprises CDR-L1, CDR-L2, and CDR-L3 sequences from a VL domain of SEQ ID NO:928 and the VH domain comprises CDR-H1, CDR-H2, and CDR-H3 from a VH domain of SEQ ID NO:929;
(cc) the VL domain comprises CDR-L1, CDR-L2, and CDR-L3 sequences from a VL domain of SEQ ID NO:930 and the VH domain comprises CDR-H1, CDR-H2, and CDR-H3 from a VH domain of SEQ ID NO:931;
(dd) the VL domain comprises CDR-L1, CDR-L2, and CDR-L3 sequences from a VL domain of SEQ ID NO932; and the VH domain comprises CDR-H1, CDR-H2, and CDR-H3 from a VH domain of SEQ ID NO:933;
(ee) the VL domain comprises CDR-L1, CDR-L2, and CDR-L3 sequences from a VL domain of SEQ ID NO:934 and the VH domain comprises CDR-H1, CDR-H2, and CDR-H3 from a VH domain of SEQ ID NO:935;
(ff) the VL domain comprises CDR-L1, CDR-L2, and CDR-L3 sequences from a VL domain of SEQ ID NO:936 and the VH domain comprises CDR-H1, CDR-H2, and CDR-H3 from a VH domain of SEQ ID NO:937;
(gg) the VL domain comprises CDR-L1, CDR-L2, and CDR-L3 sequences from a VL domain of SEQ ID NO:938 and the VH domain comprises CDR-H1, CDR-H2, and CDR-H3 from a VH domain of SEQ ID NO:939;
(hh) the VL domain comprises CDR-L1, CDR-L2, and CDR-L3 sequences from a VL domain of SEQ ID NO:940 and the VH domain comprises CDR-H1, CDR-H2, and CDR-H3 from a VH domain of SEQ ID NO:941;
(ii) the VL domain comprises CDR-L1, CDR-L2, and CDR-L3 sequences from a VL domain of SEQ ID NO:942 and the VH domain comprises CDR-H1, CDR-H2, and CDR-H3 from a VH domain of SEQ ID NO:943; or
(jj) the VL domain comprises CDR-L1, CDR-L2, and CDR-L3 sequences from a VL domain of SEQ ID NO:944 and the VH domain comprises CDR-H1, CDR-H2, and CDR-H3 from a VH domain of SEQ ID NO:945.

110-160. (canceled)

161. A pharmaceutical composition comprising the conjugate of claim 1, and a pharmaceutically acceptable carrier.

162-183. (canceled)

184. A method for treating cancer, comprising administering to an individual an effective amount of the conjugate of claim 1.

185-187. (canceled)

188. The method of claim 184, wherein the method further comprises administering at least one additional cancer therapeutic to the individual.

189. The method of claim 188, wherein the at least one additional cancer therapeutic comprises a chemotherapeutic, an immunotherapeutic, a small molecule inhibitor (SMQ), a therapeutic antibody, or a cancer vaccine.

190-191. (canceled)

192. The method of claim 184, wherein the cancer is esophageal cancer, stomach cancer, breast cancer, ovarian cancer, lung cancer, pancreatic adenocarcinoma, colon carcinoma, bladder cancer, cervical cancer, thyroid cancer, uterine cancer, rectal cancer, or gallbladder cancer.

193. (canceled)

194. A method of activating immune cells in an individual, the method comprising administering to the individual an effective amount of the conjugate of claim 1, wherein administration of the conjugate activates T cells, dendritic cells, monocytes, and/or NK cells.

195-202. (canceled)

203. A method treating a tumor or tumor cells, comprising administering an effective amount of the conjugate of claim 1.

204. (canceled)

205. A nucleic acid encoding the antibody of claim 109.

206-207. (canceled)

208. A host cell comprising the nucleic acid of claim 205.

209-210. (canceled)

211. A method of producing an antibody , comprising culturing the host cell of claim 208 under conditions to express the antibody in the host cell.

212. (canceled)

213. A method for preparing a conjugate, comprising contacting the anti-nectin-4 antibody or antigen binding fragment thereof of claim 109 with one or more immunomodulating oligonucleotides (P) in the presence of a transglutaminase, wherein the antibody or antigen binding fragment is linked to one or more Q-tag peptides (Q) comprising at least one glutamine residue.

214. A method for treating cancer, comprising administering to an individual an effective amount of the antibody of claim 109.


SEQUENCE LISTING (1117 sequences)

SEQ ID NO: 1 - DNA, 14 bp, synthetic construct
  misc_feature 1..14: note = Synthetic Construct
  modified_base 2: mod_base = OTHER; note = uridine
  modified_base 1..14: mod_base = OTHER; note = 2'-deoxynucleotides
  source 1..14: mol_type = other DNA
tncgtcgtgacgtt

SEQ ID NO: 2 - DNA, 13 bp, synthetic construct
  misc_feature 1..13: note = Synthetic Construct
  modified_base 1: mod_base = OTHER; note = uridine
  modified_base 1..13: mod_base = OTHER; note = 2'-deoxynucleotides
  source 1..13: mol_type = other DNA
ncgtcgtgtcgtt

SEQ ID NO: 3 - DNA, 13 bp, synthetic construct
  misc_feature 1..13: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker-PEG24-NH2
  modified_base 5..13: mod_base = OTHER; note = phosphorothioate linkages
  modified_base 13: mod_base = OTHER; note = 2'-O-(2-methoxyethyl)-thymidine
  misc_feature 13: note = 3' spacer C3
  source 1..13: mol_type = other DNA
ncgtcgtgtcgtt

SEQ ID NO: 4 - DNA, 13 bp, synthetic construct
  misc_feature 1..13: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4: mod_base = OTHER; note = 8-oxo-7,8-dihydro-2'-deoxyguanosine
  modified_base 4..5: mod_base = OTHER; note = phosphotriester linker-PEG24-NH2
  modified_base 5..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 5 - DNA, 13 bp, synthetic construct
  misc_feature 1..13: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 6: mod_base = OTHER; note = 8-oxo-7,8-dihydro-2'-deoxyguanosine
  modified_base 4..5: mod_base = OTHER; note = phosphotriester linker-PEG24-NH2
  modified_base 5..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 6 - DNA, 13 bp, synthetic construct
  misc_feature 1..13: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 8: mod_base = OTHER; note = 8-oxo-7,8-dihydro-2'-deoxyguanosine
  modified_base 4..5: mod_base = OTHER; note = phosphotriester linker-PEG24-NH2
  modified_base 5..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 7 - DNA, 13 bp, synthetic construct
  misc_feature 1..13: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 11: mod_base = OTHER; note = 8-oxo-7,8-dihydro-2'-deoxyguanosine
  modified_base 4..5: mod_base = OTHER; note = phosphotriester linker-PEG24-NH2
  modified_base 5..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 8 - DNA, 13 bp, synthetic construct
  misc_feature 1..13: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker-PEG24-NH2
  modified_base 5..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 9 - DNA, 14 bp, synthetic construct
  misc_feature 1..14: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker-PEG24-NH2
  modified_base 5..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 10 - DNA, 15 bp, synthetic construct
  misc_feature 1..15: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker-PEG24-NH2
  modified_base 5..15: mod_base = OTHER; note = phosphorothioate linkages
  modified_base 13..15: mod_base = OTHER; note = 2'-O-methyl-thymidine
  misc_feature 15: note = 3' spacer C3
  source 1..15: mol_type = other DNA
ncgtcgtgtcgtttt

SEQ ID NO: 11 - DNA, 13 bp, synthetic construct
  misc_feature 1..13: note = Synthetic Construct
  modified_base 1: mod_base = OTHER; note = 5-Bromo 2'-O-methyl-uridine
  modified_base 1..4: mod_base = OTHER; note = phosphorothioate linkages
  modified_base 4..5: mod_base = OTHER; note = phosphotriester linker-PEG24-NH2
  modified_base 5..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 12 - DNA, 14 bp, synthetic construct
  misc_feature 1..14: note = Synthetic Construct
  modified_base 1: mod_base = OTHER; note = 5-Bromo 2'-O-methyl-uridine
  modified_base 1..4: mod_base = OTHER; note = phosphorothioate linkages
  modified_base 4..5: mod_base = OTHER; note = phosphotriester linker-PEG24-NH2
  modified_base 5..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 13 - DNA, 14 bp, synthetic construct
  misc_feature 1..14: note = Synthetic Construct
  modified_base 1: mod_base = OTHER; note = 5-Bromo 2'-O-methyl-uridine
  modified_base 2: mod_base = OTHER; note = 2'-O-methyl-cytidine
  modified_base 1..4: mod_base = OTHER; note = phosphorothioate linkages
  modified_base 4..5: mod_base = OTHER; note = phosphotriester linker-PEG24-NH2
  modified_base 5..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 14 - DNA, 13 bp, synthetic construct
  misc_feature 1..13: note = Synthetic Construct
  modified_base 1: mod_base = OTHER; note = 5-Bromo-2'-deoxyuridine
  modified_base 1..2: mod_base = OTHER; note = phosphorothioate linkage
  modified_base 2..3: mod_base = OTHER; note = phosphorodithioate linkage
  modified_base 3..4: mod_base = OTHER; note = phosphorothioate linkage
  modified_base 4..5: mod_base = OTHER; note = phosphotriester linker
  modified_base 5..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 15 - DNA, 13 bp, synthetic construct
  misc_feature 1..13: note = Synthetic Construct
  modified_base 1: mod_base = OTHER; note = 5-Bromo-2'-deoxyuridine
  modified_base 1..3: mod_base = OTHER; note = phosphorothioate linkages
  modified_base 3..4: mod_base = OTHER; note = phosphorodithioate linkage
  modified_base 4..5: mod_base = OTHER; note = phosphotriester linker
  modified_base 5..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 16 - DNA, 13 bp, synthetic construct
  misc_feature 1..13: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker
  modified_base 5..6: mod_base = OTHER; note = phosphorodithioate linkage
  modified_base 6..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 17 - DNA, 13 bp, synthetic construct
  misc_feature 1..13: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker
  modified_base 5..6: mod_base = OTHER; note = phosphorothioate linkage
  modified_base 6..7: mod_base = OTHER; note = phosphorodithioate linkage
  modified_base 7..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 18 - DNA, 13 bp, synthetic construct
  misc_feature 1..13: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker
  modified_base 5..7: mod_base = OTHER; note = phosphorothioate linkages
  modified_base 7..8: mod_base = OTHER; note = phosphorodithioate linkage
  modified_base 8..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 19 - DNA, 13 bp, synthetic construct
  misc_feature 1..13: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker
  modified_base 5..8: mod_base = OTHER; note = phosphorothioate linkages
  modified_base 8..9: mod_base = OTHER; note = phosphorodithioate linkage
  modified_base 9..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 20 - DNA, 13 bp, synthetic construct
  misc_feature 1..13: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker
  modified_base 5..9: mod_base = OTHER; note = phosphorothioate linkages
  modified_base 9..10: mod_base = OTHER; note = phosphorodithioate linkage
  modified_base 10..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 21 - DNA, 13 bp, synthetic construct
  misc_feature 1..13: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker
  modified_base 5..10: mod_base = OTHER; note = phosphorothioate linkages
  modified_base 10..11: mod_base = OTHER; note = phosphorodithioate linkage
  modified_base 11..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 22 - DNA, 13 bp, synthetic construct
  misc_feature 1..13: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker
  modified_base 5..11: mod_base = OTHER; note = phosphorothioate linkages
  modified_base 11..12: mod_base = OTHER; note = phosphorodithioate linkage
  modified_base 12..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 23 - DNA, 13 bp, synthetic construct
  misc_feature 1..13: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker
  modified_base 5..12: mod_base = OTHER; note = phosphorothioate linkages
  modified_base 12..13: mod_base = OTHER; note = phosphorodithioate linkage
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 24 - DNA, 13 bp, synthetic construct
  misc_feature 1..13: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker
  modified_base 5..13: mod_base = OTHER; note = phosphorothioate linkages
  modified_base 13: mod_base = OTHER; note = 2'-O-methyl-uridine
  misc_feature 13: note = 3' spacer S2-C3
  source 1..13: mol_type = other DNA
ncgtcgtgtcgtn

SEQ ID NO: 25 - DNA, 13 bp, synthetic construct
  misc_feature 1..13: note = Synthetic Construct
  modified_base 1: mod_base = OTHER; note = 5-Bromo-2'-deoxyuridine
  modified_base 1..2: mod_base = OTHER; note = phosphorothioate linkage
  modified_base 2..3: mod_base = OTHER; note = phosphorodithioate linkage
  modified_base 3..4: mod_base = OTHER; note = phosphorothioate linkage
  modified_base 4..5: mod_base = OTHER; note = phosphotriester linker
  modified_base 5..13: mod_base = OTHER; note = phosphorothioate linkages
  modified_base 13: mod_base = OTHER; note = 2'-O-methyl-uridine
  misc_feature 13: note = 3' spacer S2-C3
  source 1..13: mol_type = other DNA
ncgtcgtgtcgtn

SEQ ID NO: 26 - DNA, 14 bp, synthetic construct
  misc_feature 1..14: note = Synthetic Construct
  modified_base 1: mod_base = OTHER; note = 5-Bromo 2'-O-methyl-uridine
  modified_base 2: mod_base = OTHER; note = 2'-O-methyl-cytidine
  modified_base 1..3: mod_base = OTHER; note = phosphorothioate linkages
  modified_base 3..4: mod_base = OTHER; note = phosphorodithioate linkage
  modified_base 4..5: mod_base = OTHER; note = phosphotriester linker-PEG24-NH2
  modified_base 5..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 27 - DNA, 14 bp, synthetic construct
  misc_feature 1..14: note = Synthetic Construct
  modified_base 1: mod_base = OTHER; note = 5-Bromo 2'-O-methyl-uridine
  modified_base 2: mod_base = OTHER; note = 2'-O-methyl-cytidine
  modified_base 1..3: mod_base = OTHER; note = phosphorothioate linkages
  modified_base 4..5: mod_base = OTHER; note = phosphotriester linker-PEG24-NH2
  modified_base 5..9: mod_base = OTHER; note = phosphorothioate linkages
  modified_base 9..10: mod_base = OTHER; note = phosphorodithioate linkage
  modified_base 10..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 28 - DNA, 14 bp, synthetic construct
  misc_feature 1..14: note = Synthetic Construct
  modified_base 1: mod_base = OTHER; note = 5-Bromo 2'-O-methyl-uridine
  modified_base 2: mod_base = OTHER; note = 2'-O-methyl-cytidine
  modified_base 1..3: mod_base = OTHER; note = phosphorothioate linkages
  modified_base 3..4: mod_base = OTHER; note = phosphorodithioate linkage
  modified_base 4..5: mod_base = OTHER; note = phosphotriester linker-PEG24-NH2
  modified_base 5..9: mod_base = OTHER; note = phosphorothioate linkages
  modified_base 9..10: mod_base = OTHER; note = phosphorodithioate linkage
  modified_base 10..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 29 - DNA, 14 bp, synthetic construct
  misc_feature 1..14: note = Synthetic Construct
  modified_base 1: mod_base = OTHER; note = 5-Bromo-2'-deoxyuridine
  modified_base 1..2: mod_base = OTHER; note = phosphorothioate linkage
  modified_base 2..3: mod_base = OTHER; note = phosphorodithioate linkage
  modified_base 3..4: mod_base = OTHER; note = phosphorothioate linkage
  modified_base 4..5: mod_base = OTHER; note = phosphotriester linker-PEG24-NH2
  modified_base 5..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 30 - DNA, 14 bp, synthetic construct
  misc_feature 1..14: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker-PEG24-NH2
  modified_base 5..8: mod_base = OTHER; note = phosphorothioate linkages
  modified_base 8..9: mod_base = OTHER; note = phosphorodithioate linkage
  modified_base 9..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 31 - DNA, 14 bp, synthetic construct
  misc_feature 1..14: note = Synthetic Construct
  modified_base 1: mod_base = OTHER; note = 5-Bromo-2'-deoxyuridine
  modified_base 1..2: mod_base = OTHER; note = phosphorothioate linkage
  modified_base 2..3: mod_base = OTHER; note = phosphorodithioate linkage
  modified_base 3..4: mod_base = OTHER; note = phosphorothioate linkage
  modified_base 4..5: mod_base = OTHER; note = phosphotriester linker-PEG24-NH2
  modified_base 5..8: mod_base = OTHER; note = phosphorothioate linkages
  modified_base 8..9: mod_base = OTHER; note = phosphorodithioate linkage
  modified_base 9..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 32 - DNA, 14 bp, synthetic construct
  misc_feature 1..14: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker-PEG24-NH2
  modified_base 5..6: mod_base = OTHER; note = phosphorodithioate linkage
  modified_base 6..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 33 - DNA, 14 bp, synthetic construct
  misc_feature 1..14: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker-PEG24-NH2
  modified_base 5..6: mod_base = OTHER; note = phosphorothioate linkage
  modified_base 6..7: mod_base = OTHER; note = phosphorodithioate linkage
  modified_base 7..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 34 - DNA, 14 bp, synthetic construct
  misc_feature 1..14: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker-PEG24-NH2
  modified_base 5..7: mod_base = OTHER; note = phosphorothioate linkages
  modified_base 7..8: mod_base = OTHER; note = phosphorodithioate linkage
  modified_base 8..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 35 - DNA, 14 bp, synthetic construct
  misc_feature 1..14: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker-PEG24-NH2
  modified_base 5..9: mod_base = OTHER; note = phosphorothioate linkages
  modified_base 9..10: mod_base = OTHER; note = phosphorodithioate linkage
  modified_base 10..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 36 - DNA, 14 bp, synthetic construct
  misc_feature 1..14: note = Synthetic Construct
  modified_base 1: mod_base = OTHER; note = 5-Bromo-2'-deoxyuridine
  modified_base 1..2: mod_base = OTHER; note = phosphorothioate linkage
  modified_base 2..3: mod_base = OTHER; note = phosphorodithioate linkage
  modified_base 3..4: mod_base = OTHER; note = phosphorothioate linkage
  modified_base 4..5: mod_base = OTHER; note = phosphotriester linker-PEG24-NH2
  modified_base 5..9: mod_base = OTHER; note = phosphorothioate linkages
  modified_base 9..10: mod_base = OTHER; note = phosphorodithioate linkage
  modified_base 10..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 37 - DNA, 14 bp, synthetic construct
  misc_feature 1..14: note = Synthetic Construct
  modified_base 1: mod_base = OTHER; note = 5-Bromo-2'-deoxyuridine
  modified_base 1..3: mod_base = OTHER; note = phosphorothioate linkages
  modified_base 3..4: mod_base = OTHER; note = phosphorodithioate linkage
  modified_base 4..5: mod_base = OTHER; note = phosphotriester linker-PEG24-NH2
  modified_base 5..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 38 - DNA, 14 bp, synthetic construct
  misc_feature 1..14: note = Synthetic Construct
  modified_base 1: mod_base = OTHER; note = 5-Bromo-2'-deoxyuridine
  modified_base 1..4: mod_base = OTHER; note = phosphorothioate linkages
  modified_base 4..5: mod_base = OTHER; note = phosphotriester linker-PEG24-NH2
  modified_base 5..10: mod_base = OTHER; note = phosphorothioate linkages
  modified_base 10..11: mod_base = OTHER; note = phosphorodithioate linkage
  modified_base 11..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 39 - protein, 11 aa, synthetic construct
  REGION 1..11: note = Synthetic Construct
LSLSPGLLQGG

SEQ ID NO: 40 - protein, 7 aa, synthetic construct
  REGION 1..7: note = Synthetic Construct
WPAQGPT

SEQ ID NO: 41 - protein, 6 aa, synthetic construct
  REGION 1..6: note = Synthetic Construct
WPQGPT

SEQ ID NO: 42 - protein, 7 aa, synthetic construct
  REGION 1..7: note = Synthetic Construct
WAPQGPT

SEQ ID NO: 43 - protein, 6 aa, synthetic construct
  REGION 1..6: note = Synthetic Construct
WAQGPT

SEQ ID NO: 44 - protein, 7 aa, synthetic construct
  REGION 1..7: note = Synthetic Construct
TPGQAPW

SEQ ID NO: 45 - protein, 7 aa, synthetic construct
  REGION 1..7: note = Synthetic Construct
PNPQLPF

SEQ ID NO: 46 - protein, 5 aa, synthetic construct
  REGION 1..5: note = Synthetic Construct
RPQQF

SEQ ID NO: 47 - protein, 5 aa, synthetic construct
  REGION 1..5: note = Synthetic Construct
RPQGF

SEQ ID NO: 48 - protein, 7 aa, synthetic construct
  REGION 1..7: note = Synthetic Construct
RPQGFPP

SEQ ID NO: 49 - protein, 8 aa, synthetic construct
  REGION 1..8: note = Synthetic Construct
RPQGFGPP

SEQ ID NO: 50 - protein, 7 aa, synthetic construct
  REGION 1..7: note = Synthetic Construct
RPRPQQF

SEQ ID NO: 51 - protein, 8 aa, synthetic construct
  REGION 1..8: note = Synthetic Construct
LSQSKVLG

SEQ ID NO: 52 - protein, 6 aa, synthetic construct
  REGION 1..6: note = Synthetic Construct
WGGQLL

SEQ ID NO: 53 - protein, 12 aa, synthetic construct
  REGION 1..12: note = Synthetic Construct
WALQRPHYSYPD

SEQ ID NO: 54 - protein, 12 aa, synthetic construct
  REGION 1..12: note = Synthetic Construct
WALQRPYTLTES

SEQ ID NO: 55 - protein, 12 aa, synthetic construct
  REGION 1..12: note = Synthetic Construct
WALQGPYTLTES

SEQ ID NO: 56-91 - skipped (36 entries)

SEQ ID NO: 92 - protein, 329 aa, synthetic construct
  REGION 1..329: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 93 - protein, 329 aa, synthetic construct
  REGION 1..329: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 329 aa, synthetic construct
  REGION 1..329: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 337 aa, synthetic construct
  REGION 1..337: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 96 - protein, 329 aa, synthetic construct
  REGION 1..329: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 97 - protein, 329 aa, synthetic construct
  REGION 1..329: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 329 aa, synthetic construct
  REGION 1..329: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 325 aa, synthetic construct
  REGION 1..325: note = Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 100 - protein, 325 aa, synthetic construct
  REGION 1..325: note = Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 101 - protein, 325 aa, synthetic construct
  REGION 1..325: note = Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFASTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 102 - protein, 325 aa, synthetic construct
  REGION 1..325: note = Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFASTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 103 - protein, 325 aa, synthetic construct
  REGION 1..325: note = Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 104 - protein, 326 aa, synthetic construct
  REGION 1..326: note = Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 105 - protein, 326 aa, synthetic construct
  REGION 1..326: note = Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 106 - protein, 326 aa, synthetic construct
  REGION 1..326: note = Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 107 - protein, 326 aa, synthetic construct
  REGION 1..326: note = Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFASTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 108 - protein, 107 aa, Homo sapiens [sequence omitted]

SEQ ID NO: 109 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 110 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 111 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 112 - protein, 337 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 113-128 - skipped (16 entries)

SEQ ID NO: 129 - DNA, 24 bp, synthetic construct
  misc_feature 1..24: note = Synthetic Construct
  modified_base 1..24: mod_base = OTHER; note = 2'-deoxynucleotides
  source 1..24: mol_type = other DNA
tcgtcgttttgtcgttttgtcgtt

SEQ ID NO: 130 - DNA, 13 bp, synthetic construct
  misc_feature 1..13: note = Synthetic Construct
  modified_base 1..4: mod_base = OTHER; note = phosphorothioate linkages
  modified_base 4..5: mod_base = OTHER; note = phosphotriester linker-PEG24-NH2
  modified_base 5..13: mod_base = OTHER; note = phosphorothioate linkages
  modified_base 13: mod_base = OTHER; note = 2'-O-(2-methoxyethyl)-thymidine
  misc_feature 13: note = 3' spacer C3
  source 1..13: mol_type = other DNA
ncgtcgtgtcgtt

SEQ ID NO: 131 - DNA, 13 bp, synthetic construct
  misc_feature 1..13: note = Synthetic Construct
  modified_base 1..4: mod_base = OTHER; note = phosphorothioate linkages
  modified_base 3: mod_base = OTHER; note = 8-oxo-7,8-dihydro-2'-deoxyguanosine
  modified_base 4..5: mod_base = OTHER; note = phosphotriester linker-PEG24-NH2
  modified_base 5..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 132 - DNA, 13 bp, synthetic construct
  misc_feature 1..13: note = Synthetic Construct
  modified_base 1..4: mod_base = OTHER; note = phosphorothioate linkages
  modified_base 4..5: mod_base = OTHER; note = phosphotriester linker-PEG24-NH2
  modified_base 5..13: mod_base = OTHER; note = phosphorothioate linkages
  modified_base 6: mod_base = OTHER; note = 8-oxo-7,8-dihydro-2'-deoxyguanosine
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 133 - DNA, 13 bp, synthetic construct
  misc_feature 1..13: note = Synthetic Construct
  modified_base 1..4: mod_base = OTHER; note = phosphorothioate linkages
  modified_base 4..5: mod_base = OTHER; note = phosphotriester linker-PEG24-NH2
  modified_base 5..13: mod_base = OTHER; note = phosphorothioate linkages
  modified_base 8: mod_base = OTHER; note = 8-oxo-7,8-dihydro-2'-deoxyguanosine
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 134 - DNA, 13 bp, synthetic construct
  misc_feature 1..13: note = Synthetic Construct
  modified_base 1..4: mod_base = OTHER; note = phosphorothioate linkages
  modified_base 4..5: mod_base = OTHER; note = phosphotriester linker-PEG24-NH2
  modified_base 5..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 135 - DNA, 13 bp, synthetic construct
  misc_feature 1..13: note = Synthetic Construct
  modified_base 1..4: mod_base = OTHER; note = phosphorothioate linkages
  modified_base 4..5: mod_base = OTHER; note = phosphotriester linker-PEG24-NH2
  modified_base 5..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 136 - DNA, 14 bp, synthetic construct
  misc_feature 1..14: note = Synthetic Construct
  modified_base 1..4: mod_base = OTHER; note = phosphorothioate linkages
  modified_base 4..5: mod_base = OTHER; note = phosphotriester linker-PEG24-NH2
  modified_base 5..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 137 - DNA, 15 bp, synthetic construct
  misc_feature 1..15: note = Synthetic Construct
  modified_base 1..4: mod_base = OTHER; note = phosphorothioate linkages
  modified_base 4..5: mod_base = OTHER; note = phosphotriester linker-PEG24-NH2
  modified_base 5..15: mod_base = OTHER; note = phosphorothioate linkages
  modified_base 13..15: mod_base = OTHER; note = 2'-O-methyl-thymidine
  misc_feature 15: note = 3' spacer C3
  source 1..15: mol_type = other DNA
ncgtcgtgtcgtttt

SEQ ID NO: 138 - DNA, 13 bp, synthetic construct
  misc_feature 1..13: note = Synthetic Construct
  modified_base 1: mod_base = OTHER; note = 2'-O-methyl-uridine
  modified_base 1..4: mod_base = OTHER; note = phosphorothioate linkages
  modified_base 4..5: mod_base = OTHER; note = phosphotriester linker-PEG24-NH2
  modified_base 5..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 139 - DNA, 14 bp, synthetic construct
  misc_feature 1..14: note = Synthetic Construct
  modified_base 1: mod_base = OTHER; note = 2'-O-methyl-uridine
  modified_base 1..4: mod_base = OTHER; note = phosphorothioate linkages
  modified_base 4..5: mod_base = OTHER; note = phosphotriester linker-PEG24-NH2
  modified_base 5..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 140 - DNA, 14 bp, synthetic construct
  misc_feature 1..14: note = Synthetic Construct
  modified_base 1: mod_base = OTHER; note = 2'-O-methyl-uridine
  modified_base 2: mod_base = OTHER; note = 2'-O-methyl-cytidine
  modified_base 1..4: mod_base = OTHER; note = phosphorothioate linkages
  modified_base 4..5: mod_base = OTHER; note = phosphotriester linker-PEG24-NH2
  modified_base 5..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 141 - DNA, 13 bp, synthetic construct
  misc_feature 1..13: note = Synthetic Construct
  modified_base 1..2: mod_base = OTHER; note = phosphorothioate linkage
  modified_base 2..3: mod_base = OTHER; note = phosphorodithioate linkage
  modified_base 3..4: mod_base = OTHER; note = phosphorothioate linkage
  modified_base 4..5: mod_base = OTHER; note = phosphotriester linker
  modified_base 5..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 142 - DNA, 13 bp, synthetic construct
  misc_feature 1..13: note = Synthetic Construct
  modified_base 1..3: mod_base = OTHER; note = phosphorothioate linkages
  modified_base 3..4: mod_base = OTHER; note = phosphorodithioate linkage
  modified_base 4..5: mod_base = OTHER; note = phosphotriester linker
  modified_base 5..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 143 - DNA, 13 bp, synthetic construct
  misc_feature 1..13: note = Synthetic Construct
  modified_base 1..4: mod_base = OTHER; note = phosphorothioate linkages
  modified_base 4..5: mod_base = OTHER; note = phosphotriester linker
  modified_base 5..6: mod_base = OTHER; note = phosphorodithioate linkage
  modified_base 6..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 144 - DNA, 13 bp, synthetic construct
  misc_feature 1..13: note = Synthetic Construct
  modified_base 1..4: mod_base = OTHER; note = phosphorothioate linkages
  modified_base 4..5: mod_base = OTHER; note = phosphotriester linker
  modified_base 5..6: mod_base = OTHER; note = phosphorothioate linkage
  modified_base 6..7: mod_base = OTHER; note = phosphorodithioate linkage
  modified_base 6..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 145 - DNA, 13 bp, synthetic construct
  misc_feature 1..13: note = Synthetic Construct
  modified_base 1..4: mod_base = OTHER; note = phosphorothioate linkages
  modified_base 4..5: mod_base = OTHER; note = phosphotriester linker
  modified_base 5..7: mod_base = OTHER; note = phosphorothioate linkages
  modified_base 7..8: mod_base = OTHER; note = phosphorodithioate linkage
  modified_base 8..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 146 - DNA, 13 bp, synthetic construct
  misc_feature 1..13: note = Synthetic Construct
  modified_base 1..4: mod_base = OTHER; note = phosphorothioate linkages
  modified_base 4..5: mod_base = OTHER; note = phosphotriester linker
  modified_base 5..8: mod_base = OTHER; note = phosphorothioate linkages
  modified_base 8..9: mod_base = OTHER; note = phosphorodithioate linkage
  modified_base 9..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 147 - DNA, 13 bp, synthetic construct
  misc_feature 1..13: note = Synthetic Construct
  modified_base 1..4: mod_base = OTHER; note = phosphorothioate linkages
  modified_base 4..5: mod_base = OTHER; note = phosphotriester linker
  modified_base 5..9: mod_base = OTHER; note = phosphorothioate linkages
  modified_base 9..10: mod_base = OTHER; note = phosphorodithioate linkage
  modified_base 10..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 148 - DNA, 13 bp, synthetic construct
  misc_feature 1..13: note = Synthetic Construct
  modified_base 1..4: mod_base = OTHER; note = phosphorothioate linkages
  modified_base 4..5: mod_base = OTHER; note = phosphotriester linker
  modified_base 5..10: mod_base = OTHER; note = phosphorothioate linkages
  modified_base 10..11: mod_base = OTHER; note = phosphorodithioate linkage
  modified_base 11..13: mod_base = OTHER; note = phosphorothioate linkages
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 149 - DNA, 13 bp, synthetic construct
  misc_feature 1..13: note = Synthetic Construct
  modified_base 1..4: mod_base = OTHER; note = phosphorothioate linkages
  modified_base 4..5: mod_base = OTHER; note = phosphotriester linker
  modified_base 5..11: mod_base = OTHER; note = phosphorothioate linkages
  modified_base 11..12: mod_base = OTHER; note = phosphorodithioate linkage
  modified_base 12..13: mod_base = OTHER; note = phosphorothioate linkage
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 150 - DNA, 13 bp, synthetic construct
  misc_feature 1..13: note = Synthetic Construct
  modified_base 1..4: mod_base = OTHER; note = phosphorothioate linkages
  modified_base 4..5: mod_base = OTHER; note = phosphotriester linker
  modified_base 5..12: mod_base = OTHER; note = phosphorothioate linkages
  modified_base 12..13: mod_base = OTHER; note = phosphorodithioate linkage
  modified_base 13: mod_base = OTHER; note = 2'-O-methyl-thymidine
  misc_feature 13: note = 3' spacer C3
  source 1..13: mol_type = other DNA
ncgtcgtgtcgtt

SEQ ID NO: 151 - DNA, 13 bp, synthetic construct
  misc_feature 1..13: note = Synthetic Construct
  modified_base 1..4: mod_base = OTHER; note = phosphorothioate linkages
  modified_base 4..5: mod_base = OTHER; note = phosphotriester linker
  modified_base 5..13: mod_base = OTHER; note = phosphorothioate linkages
  modified_base 13: mod_base = OTHER; note = 2'-O-methyl-uridine
  misc_feature 13: note = 3' spacer S2-C3
  source 1..13: mol_type = other DNA
ncgtcgtgtcgtn

SEQ ID NO: 152 - DNA, 13 bp, synthetic construct
  misc_feature 1..13: note = Synthetic Construct
  modified_base 1..2: mod_base = OTHER; note = phosphorothioate linkage
  modified_base 2..3: mod_base = OTHER; note = phosphorodithioate linkage
  modified_base 3..4: mod_base = OTHER; note = phosphorothioate linkage
  modified_base 4..5: mod_base = OTHER; note = phosphotriester linker
  modified_base 5..13: mod_base = OTHER; note = phosphorothioate linkages
  modified_base 13: mod_base = OTHER; note = 2'-O-methyl-uridine
  misc_feature 13: note = 3' spacer S2-C3
  source 1..13: mol_type = other DNA
ncgtcgtgtcgtn

SEQ ID NO: 153 - DNA, 14 bp, synthetic construct
  misc_feature 1..14: note = Synthetic Construct
  modified_base 1: mod_base = OTHER; note = 2'-O-methyl-uridine
  modified_base 2: mod_base = OTHER; note = 2'-O-methyl-cytidine
  modified_base 1..3: mod_base = OTHER; note = phosphorothioate linkages
  modified_base 3..4: mod_base = OTHER; note = phosphorodithioate linkage
  modified_base 4..5: mod_base = OTHER; note = phosphotriester linker-PEG24-NH2
  modified_base 5..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 154 - DNA, 14 bp, synthetic construct
  misc_feature 1..14: note = Synthetic Construct
  modified_base 1: mod_base = OTHER; note = 2'-O-methyl-uridine
  modified_base 2: mod_base = OTHER; note = 2'-O-methyl-cytidine
  modified_base 1..4: mod_base = OTHER; note = phosphorothioate linkages
  modified_base 4..5: mod_base = OTHER; note = phosphotriester linker-PEG24-NH2
  modified_base 5..9: mod_base = OTHER; note = phosphorothioate linkages
  modified_base 9..10: mod_base = OTHER; note = phosphorodithioate linkage
  modified_base 10..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 155 - DNA, 14 bp, synthetic construct
  misc_feature 1..14: note = Synthetic Construct
  modified_base 1: mod_base = OTHER; note = 2'-O-methyl-uridine
  modified_base 2: mod_base = OTHER; note = 2'-O-methyl-cytidine
  modified_base 1..3: mod_base = OTHER; note = phosphorothioate linkages
  modified_base 3..4: mod_base = OTHER; note = phosphorodithioate linkage
  modified_base 4..5: mod_base = OTHER; note = phosphotriester linker-PEG24-NH2
  modified_base 5..9: mod_base = OTHER; note = phosphorothioate linkages
  modified_base 9..10: mod_base = OTHER; note = phosphorodithioate linkage
  modified_base 10..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 156 - DNA, 14 bp, synthetic construct
  misc_feature 1..14: note = Synthetic Construct
  modified_base 1..2: mod_base = OTHER; note = phosphorothioate linkage
  modified_base 2..3: mod_base = OTHER; note = phosphorodithioate linkage
  modified_base 3..4: mod_base = OTHER; note = phosphorothioate linkage
  modified_base 4..5: mod_base = OTHER; note = phosphotriester linker-PEG24-NH2
  modified_base 5..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 157 - DNA, 14 bp, synthetic construct
  misc_feature 1..14: note = Synthetic Construct
  modified_base 1..4: mod_base = OTHER; note = phosphorothioate linkages
  modified_base 4..5: mod_base = OTHER; note = phosphotriester linker-PEG24-NH2
  modified_base 5..8: mod_base = OTHER; note = phosphorothioate linkages
  modified_base 8..9: mod_base = OTHER; note = phosphorodithioate linkage
  modified_base 9..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 158 - DNA, 14 bp, synthetic construct
  misc_feature 1..14: note = Synthetic Construct
  modified_base 1..2: mod_base = OTHER; note = phosphorothioate linkage
  modified_base 2..3: mod_base = OTHER; note = phosphorodithioate linkage
  modified_base 3..4: mod_base = OTHER; note = phosphorothioate linkage
  modified_base 4..5: mod_base = OTHER; note = phosphotriester linker-PEG24-NH2
  modified_base 5..8: mod_base = OTHER; note = phosphorothioate linkages
  modified_base 8..9: mod_base = OTHER; note = phosphorodithioate linkage
  modified_base 9..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 159 - DNA, 14 bp, synthetic construct
  misc_feature 1..14: note = Synthetic Construct
  modified_base 1..4: mod_base = OTHER; note = phosphorothioate linkages
  modified_base 4..5: mod_base = OTHER; note = phosphotriester linker-PEG24-NH2
  modified_base 5..6: mod_base = OTHER; note = phosphorodithioate linkage
  modified_base 6..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 160 - DNA, 14 bp, synthetic construct
  misc_feature 1..14: note = Synthetic Construct
  modified_base 1..4: mod_base = OTHER; note = phosphorothioate linkages
  modified_base 4..5: mod_base = OTHER; note = phosphotriester linker-PEG24-NH2
  modified_base 5..6: mod_base = OTHER; note = phosphorothioate linkage
  modified_base 6..7: mod_base = OTHER; note = phosphorodithioate linkage
  modified_base 7..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 161 - DNA, 14 bp, synthetic construct
  misc_feature 1..14: note = Synthetic Construct
  modified_base 1..4: mod_base = OTHER; note = phosphorothioate linkages
  modified_base 4..5: mod_base = OTHER; note = phosphotriester linker-PEG24-NH2
  modified_base 5..7: mod_base = OTHER; note = phosphorothioate linkages
  modified_base 7..8: mod_base = OTHER; note = phosphorodithioate linkage
  modified_base 8..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 162 - DNA, 14 bp, synthetic construct
  misc_feature 1..14: note = Synthetic Construct
  modified_base 1..4: mod_base = OTHER; note = phosphorothioate linkages
  modified_base 4..5: mod_base = OTHER; note = phosphotriester linker
  modified_base 5..9: mod_base = OTHER; note = phosphorothioate linkages
  modified_base 9..10: mod_base = OTHER; note = phosphorodithioate linkage
  modified_base 10..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 163 - DNA, 14 bp, synthetic construct
  misc_feature 1..14: note = Synthetic Construct
  modified_base 1..4: mod_base = OTHER; note = phosphorothioate linkages
  modified_base 4..5: mod_base = OTHER; note = phosphotriester linker-PEG24-NH2
  modified_base 5..9: mod_base = OTHER; note = phosphorothioate linkages
  modified_base 9..10: mod_base = OTHER; note = phosphorodithioate linkage
  modified_base 10..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 164 - DNA, 14 bp, synthetic construct
  misc_feature 1..14: note = Synthetic Construct
  modified_base 1..2: mod_base = OTHER; note = phosphorothioate linkage
  modified_base 2..3: mod_base = OTHER; note = phosphorodithioate linkage
  modified_base 3..4: mod_base = OTHER; note = phosphorothioate linkage
  modified_base 4..5: mod_base = OTHER; note = phosphotriester linker-PEG24-NH2
  modified_base 5..9: mod_base = OTHER; note = phosphorothioate linkages
  modified_base 9..10: mod_base = OTHER; note = phosphorodithioate linkage
  modified_base 10..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 165 - DNA, 14 bp, synthetic construct
  misc_feature 1..14: note = Synthetic Construct
  modified_base 1..3: mod_base = OTHER; note = phosphorothioate linkages
  modified_base 3..4: mod_base = OTHER; note = phosphorodithioate linkage
  modified_base 4..5: mod_base = OTHER; note = phosphotriester linker-PEG24-NH2
  modified_base 5..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 166 - DNA, 14 bp, synthetic construct
  misc_feature 1..14: note = Synthetic Construct
  modified_base 1..4: mod_base = OTHER; note = phosphorothioate linkages
  modified_base 4..5: mod_base = OTHER; note = phosphotriester linker-PEG24-NH2
  modified_base 5..10: mod_base = OTHER; note = phosphorothioate linkages
  modified_base 10..11: mod_base = OTHER; note = phosphorodithioate linkage
  modified_base 11..14: mod_base = OTHER; note = phosphorothioate linkages
  modified_base 13..14: mod_base = OTHER; note = 2'-O-methyl-thymidine
  misc_feature 14: note = 3' spacer C3
  source 1..14: mol_type = other DNA
ncgtcgtgtcgttt

SEQ ID NO: 167 - DNA, 24 bp, synthetic construct
  misc_feature 1..24: note = Synthetic Construct
  source 1..24: mol_type = other DNA
tcgtcgttttgtcgttttgtcgtt

SEQ ID NO: 168-171 - skipped (4 entries)

SEQ ID NO: 172 - protein, 5 aa, synthetic construct
  REGION 1..5: note = Synthetic Construct
LLQGG

SEQ ID NO: 173 - protein, 8 aa, synthetic construct
  REGION 1..8: note = Synthetic Construct
GGGLLQGG

SEQ ID NO: 174-175 - skipped (2 entries)

SEQ ID NO: 176 - DNA, 20 bp, synthetic construct
  misc_feature 1..20: note = Synthetic Construct
  source 1..20: mol_type = other DNA
tccatgacgttcctgacgtt

SEQ ID NO: 177 - skipped

SEQ ID NO: 178 - protein, 337 aa, synthetic construct
  REGION 1..337: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 179-199 - skipped (21 entries)

SEQ ID NO: 200 - protein, 458 aa, synthetic construct
  REGION 1..458: note = Synthetic Construct
EVQLVQSGAEVKKPGSSVKVSCKASGYTFTGYYMHWVRQAPGQGLEWMGWINPNSGGTNYAQKFQGRVTMTRDTSISTAYMELRSLRSDDTAVYYCARGSALHFFGVFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGRPQGFGPP

SEQ ID NO: 201 - protein, 460 aa, synthetic construct
  REGION 1..460: note = Synthetic Construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYSSSWYEPYYFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGRPQGFGPP

SEQ ID NO: 202 - protein, 455 aa, synthetic construct
  REGION 1..455: note = Synthetic Construct
EVQLVQSGGGLVKPGGSLRLSCAASGFTFSDYYMSWVRQAPGKGLEWVSAISGSGGSTYYADSVKGRFTISRDNSKNTLYLQMNSLRAEDTAVYYCARDEGYYYMDVWGKGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGRPQGFGPP

SEQ ID NO: 203 - protein, 465 aa, synthetic construct
  REGION 1..465: note = Synthetic Construct
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKSSYYDYVWGSYRYTDAFDIWGQGTMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGRPQGFGPP

SEQ ID NO: 204 - protein, 457 aa, synthetic construct
  REGION 1..457: note = Synthetic Construct
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDYGYNWNFFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGRPQGFGPP

SEQ ID NO: 205 - protein, 459 aa, synthetic construct
  REGION 1..459: note = Synthetic Construct
QVQLQESGPGLVKPSGTLSLTCTVSGGSISSGGYYWAWIRQPPGKGLEWIGSIYYTGSTYYNPSLKSRVTIYVDTSKNQFSLKLSSVTAADTAVYYCARDPFPEPPDAFDIWGQGTMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGRPQGFGPP

SEQ ID NO: 206 - protein, 456 aa, synthetic construct
  REGION 1..456: note = Synthetic Construct
EVQLVQSGAEVKKPGASVKVSCKASGYTFTSYDINWVRQAPGQGLEWMGGIIPIFGTANYAQKFQGRVTITADESTSTAYMELRSLRSEDTAVYYCASTAVAGFGIDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGRPQGFGPP

SEQ ID NO: 207 - protein, 464 aa, synthetic construct
  REGION 1..464: note = Synthetic Construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAAGYYYDSSGYYHQPYYFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGRPQGFGPP

SEQ ID NO: 208 - protein, 456 aa, synthetic construct
  REGION 1..456: note = Synthetic Construct
EVQLVESGGGLVQPGGSLRLSCAASGFTFSDYYMSWIRQAPGKGLEWVSGISWNSGSIGYADSVKGRFTISRDNSKNTLYLQMNSLRAEDTAVYYCASSGSWYLPLDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGRPQGFGPP

SEQ ID NO: 209 - protein, 458 aa, synthetic construct
  REGION 1..458: note = Synthetic Construct
EVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARATLQLWLPLFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGRPQGFGPP

SEQ ID NO: 210 - protein, 457 aa, synthetic construct
  REGION 1..457: note = Synthetic Construct
EVQLVQSGAEVKKPGSSVKVSCKASGGTFSSYAISWVRQAPGQGLEWMGWINPNSGGTNYAQKFQGRVTMTRDTSTSTAYMELSSLRSEDTAVYYCATGYYGDLYYFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGRPQGFGPP

SEQ ID NO: 211 - protein, 451 aa, synthetic construct
  REGION 1..451: note = Synthetic Construct
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGLLQGG

SEQ ID NO: 212 - protein, 214 aa, synthetic construct
  REGION 1..214: note = Synthetic Construct
DIVMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 213 - protein, 214 aa, synthetic construct
  REGION 1..214: note = Synthetic Construct
DIQLTQSPSSLSASVGDRVTITCRASQGISNYLAWYQQKPGKAPKLLIYAASTLQSGVPSRFSGSGSGTDFTLTISSLQPEDFATYYCQQSYSTPYTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 214 - protein, 212 aa, synthetic construct
  REGION 1..212: note = Synthetic Construct
EIVLTQSPATLSLSPGERATLSCRASQSVSSYLAWYQQKPGQAPRLLIYDASNRATGIPARFSGSGSGTDFTLTISSLQAEDFATYYCQQSYSTRFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 215 - protein, 214 aa, synthetic construct
  REGION 1..214: note = Synthetic Construct
EIVLTQSPATLSLSPGERATLSCRASQSVSSSYLAWYQQKPGQAPRLLIYGASSRATGIPDRFSGSGSGTDFTLTISRLEPEDFAVYYCQQYNNWWTFGQGTKVDIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 216 - protein, 214 aa, synthetic construct
  REGION 1..214: note = Synthetic Construct
DIVMTQSPSSLSASVGDRVTITCRASQGISNYLAWYQQKPGKVPKLLIYAASTLQSGVPSRFSGSGSGTDFTLTISSLQPEDVATYYCQKYNSAPYTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 217 - protein, 214 aa, synthetic construct
  REGION 1..214: note = Synthetic Construct
EIVLTQSPATLSVSPGERATLSCRASQSVSSNLAWYQQKPGQAPRLLIYGASSRATGIPDRFSGSGSGTDFTLTISRLEPEDFAVYYCHQYGSSPLTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 218 - protein, 214 aa, synthetic construct
  REGION 1..214: note = Synthetic Construct
DIQMTQSPSSLSASVGDRVTITCRASQGISNYLAWFQQKPGKAPKSLISAASSLQSGVPSRFSGSGSGTDFTLTISSLQPEDFATYYCLQHDSFPITFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 219 - protein, 214 aa, synthetic construct
  REGION 1..214: note = Synthetic Construct
DIQMTQSPSTLSASVGDRVTITCRASQGISNYLAWYQQKPGKAPKLLIYAASSLQSGVPSRFSGSGSGTDFTLTISSLQPEDFATYYCQQSYSTPLTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 220 - protein, 214 aa, synthetic construct
  REGION 1..214: note = Synthetic Construct
DIQMTQSPSSLSASVGDRVTITCQASQDISNYLNWYQQKPGKVPKLLIYAASTLQSGVPSRFSGSGSGTDFTLTISSLQPEDFATYYCQQSYSTPLTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 221 - protein, 214 aa, synthetic construct
  REGION 1..214: note = Synthetic Construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSLTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 222 - protein, 214 aa, synthetic construct
  REGION 1..214: note = Synthetic Construct
DIVMTQSPSSLSASVGDRVTITCRASQGISNYLAWYQQKPGKVPKLLIYAASTLQSGVPSRFSGSGSGTEFTLAISSLQPEDLATYYCQQLNSHPITFGQGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 223 - protein, 214 aa, synthetic construct
  REGION 1..214: note = Synthetic Construct
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 224 - protein, 458 aa, synthetic construct
  REGION 1..458: note = Synthetic Construct
EVQLVQSGAEVKKPGSSVKVSCKASGYTFTGYYMHWVRQAPGQGLEWMGWINPNSGGTNYAQKFQGRVTMTRDTSISTAYMELRSLRSDDTAVYYCARGSALHFFGVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 225 - protein, 460 aa, synthetic construct
  REGION 1..460: note = Synthetic Construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYSSSWYEPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 226 - protein, 455 aa, synthetic construct
  REGION 1..455: note = Synthetic Construct
EVQLVQSGGGLVKPGGSLRLSCAASGFTFSDYYMSWVRQAPGKGLEWVSAISGSGGSTYYADSVKGRFTISRDNSKNTLYLQMNSLRAEDTAVYYCARDEGYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 227 - protein, 465 aa, synthetic construct
  REGION 1..465: note = Synthetic Construct
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKSSYYDYVWGSYRYTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 228 - protein, 457 aa, synthetic construct
  REGION 1..457: note = Synthetic Construct
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDYGYNWNFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 229 - protein, 459 aa, synthetic construct
  REGION 1..459: note = Synthetic Construct
QVQLQESGPGLVKPSGTLSLTCTVSGGSISSGGYYWAWIRQPPGKGLEWIGSIYYTGSTYYNPSLKSRVTIYVDTSKNQFSLKLSSVTAADTAVYYCARDPFPEPPDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 230 - protein, 456 aa, synthetic construct
  REGION 1..456: note = Synthetic Construct
EVQLVQSGAEVKKPGASVKVSCKASGYTFTSYDINWVRQAPGQGLEWMGGIIPIFGTANYAQKFQGRVTITADESTSTAYMELRSLRSEDTAVYYCASTAVAGFGIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 231 - protein, 464 aa, synthetic construct
  REGION 1..464: note = Synthetic Construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAAGYYYDSSGYYHQPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 232 - protein, 456 aa, synthetic construct
  REGION 1..456: note = Synthetic Construct
EVQLVESGGGLVQPGGSLRLSCAASGFTFSDYYMSWIRQAPGKGLEWVSGISWNSGSIGYADSVKGRFTISRDNSKNTLYLQMNSLRAEDTAVYYCASSGSWYLPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 233 - protein, 458 aa, synthetic construct
  REGION 1..458: note = Synthetic Construct
EVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARATLQLWLPLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 234 - protein, 457 aa, synthetic construct
  REGION 1..457: note = Synthetic Construct
EVQLVQSGAEVKKPGSSVKVSCKASGGTFSSYAISWVRQAPGQGLEWMGWINPNSGGTNYAQKFQGRVTMTRDTSTSTAYMELSSLRSEDTAVYYCATGYYGDLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 235 - protein, 454 aa, synthetic construct
  REGION 1..454: note = Synthetic Construct
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 236 - protein, 214 aa, synthetic construct
  REGION 1..214: note = Synthetic Construct
DIVMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - protein, 214 aa, synthetic construct
  REGION 1..214: note = Synthetic Construct
DIQLTQSPSSLSASVGDRVTITCRASQGISNYLAWYQQKPGKAPKLLIYAASTLQSGVPSRFSGSGSGTDFTLTISSLQPEDFATYYCQQSYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 212 aa, synthetic construct
  REGION 1..212: note = Synthetic Construct
EIVLTQSPATLSLSPGERATLSCRASQSVSSYLAWYQQKPGQAPRLLIYDASNRATGIPARFSGSGSGTDFTLTISSLQAEDFATYYCQQSYSTRFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 239 - protein, 214 aa, synthetic construct
  REGION 1..214: note = Synthetic Construct
EIVLTQSPATLSLSPGERATLSCRASQSVSSSYLAWYQQKPGQAPRLLIYGASSRATGIPDRFSGSGSGTDFTLTISRLEPEDFAVYYCQQYNNWWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 240 - protein, 214 aa, synthetic construct
  REGION 1..214: note = Synthetic Construct
DIVMTQSPSSLSASVGDRVTITCRASQGISNYLAWYQQKPGKVPKLLIYAASTLQSGVPSRFSGSGSGTDFTLTISSLQPEDVATYYCQKYNSAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 241 - protein, 214 aa, synthetic construct
  REGION 1..214: note = Synthetic Construct
EIVLTQSPATLSVSPGERATLSCRASQSVSSNLAWYQQKPGQAPRLLIYGASSRATGIPDRFSGSGSGTDFTLTISRLEPEDFAVYYCHQYGSSPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 242 - protein, 214 aa, synthetic construct
  REGION 1..214: note = Synthetic Construct
DIQMTQSPSSLSASVGDRVTITCRASQGISNYLAWFQQKPGKAPKSLISAASSLQSGVPSRFSGSGSGTDFTLTISSLQPEDFATYYCLQHDSFPITFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 243 - protein, 214 aa, synthetic construct
  REGION 1..214: note = Synthetic Construct
DIQMTQSPSTLSASVGDRVTITCRASQGISNYLAWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 214 aa, synthetic construct
  REGION 1..214: note = Synthetic Construct
DIQMTQSPSSLSASVGDRVTITCQASQDISNYLNWYQQKPGKVPKLLIYAASTLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245 - protein, 214 aa, synthetic construct
  REGION 1..214: note = Synthetic Construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - protein, 214 aa, synthetic construct
  REGION 1..214: note = Synthetic Construct
DIVMTQSPSSLSASVGDRVTITCRASQGISNYLAWYQQKPGKVPKLLIYAASTLQSGVPSRFSGSGSGTEFTLAISSLQPEDLATYYCQQLNSHPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 247 - protein, 214 aa, synthetic construct
  REGION 1..214: note = Synthetic Construct
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 248 - protein, 107 aa, synthetic construct
  REGION 1..107: note = Synthetic Construct
DIVMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 249 - protein, 121 aa, synthetic construct
  REGION 1..121: note = Synthetic Construct
EVQLVQSGAEVKKPGSSVKVSCKASGYTFTGYYMHWVRQAPGQGLEWMGWINPNSGGTNYAQKFQGRVTMTRDTSISTAYMELRSLRSDDTAVYYCARGSALHFFGVFDYWGQGTLVTVSS

SEQ ID NO: 250 - protein, 107 aa, synthetic construct
  REGION 1..107: note = Synthetic Construct
DIQLTQSPSSLSASVGDRVTITCRASQGISNYLAWYQQKPGKAPKLLIYAASTLQSGVPSRFSGSGSGTDFTLTISSLQPEDFATYYCQQSYSTPYTFGQGTKLEIK

SEQ ID NO: 251 - protein, 123 aa, synthetic construct
  REGION 1..123: note = Synthetic Construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYSSSWYEPYYFDYWGQGTLVTVSS

SEQ ID NO: 252 - protein, 105 aa, synthetic construct
  REGION 1..105: note = Synthetic Construct
EIVLTQSPATLSLSPGERATLSCRASQSVSSYLAWYQQKPGQAPRLLIYDASNRATGIPARFSGSGSGTDFTLTISSLQAEDFATYYCQQSYSTRFGGGTKVEIK

SEQ ID NO: 253 - protein, 118 aa, synthetic construct
  REGION 1..118: note = Synthetic Construct
EVQLVQSGGGLVKPGGSLRLSCAASGFTFSDYYMSWVRQAPGKGLEWVSAISGSGGSTYYADSVKGRFTISRDNSKNTLYLQMNSLRAEDTAVYYCARDEGYYYMDVWGKGTTVTVSS

SEQ ID NO: 254 - protein, 107 aa, synthetic construct
  REGION 1..107: note = Synthetic Construct
EIVLTQSPATLSLSPGERATLSCRASQSVSSSYLAWYQQKPGQAPRLLIYGASSRATGIPDRFSGSGSGTDFTLTISRLEPEDFAVYYCQQYNNWWTFGQGTKVDIK

SEQ ID NO: 255 - protein, 128 aa, synthetic construct
  REGION 1..128: note = Synthetic Construct
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKSSYYDYVWGSYRYTDAFDIWGQGTMVTVSS

SEQ ID NO: 256 - protein, 107 aa, synthetic construct
  REGION 1..107: note = Synthetic Construct
DIVMTQSPSSLSASVGDRVTITCRASQGISNYLAWYQQKPGKVPKLLIYAASTLQSGVPSRFSGSGSGTDFTLTISSLQPEDVATYYCQKYNSAPYTFGQGTKVEIK

SEQ ID NO: 257 - protein, 120 aa, synthetic construct
  REGION 1..120: note = Synthetic Construct
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDYGYNWNFFDYWGQGTLVTVSS

SEQ ID NO: 258 - protein, 107 aa, synthetic construct
  REGION 1..107: note = Synthetic Construct
EIVLTQSPATLSVSPGERATLSCRASQSVSSNLAWYQQKPGQAPRLLIYGASSRATGIPDRFSGSGSGTDFTLTISRLEPEDFAVYYCHQYGSSPLTFGGGTKLEIK

SEQ ID NO: 259 - protein, 122 aa, synthetic construct
  REGION 1..122: note = Synthetic Construct
QVQLQESGPGLVKPSGTLSLTCTVSGGSISSGGYYWAWIRQPPGKGLEWIGSIYYTGSTYYNPSLKSRVTIYVDTSKNQFSLKLSSVTAADTAVYYCARDPFPEPPDAFDIWGQGTMVTVSS

SEQ ID NO: 260 - protein, 107 aa, synthetic construct
  REGION 1..107: note = Synthetic Construct
DIQMTQSPSSLSASVGDRVTITCRASQGISNYLAWFQQKPGKAPKSLISAASSLQSGVPSRFSGSGSGTDFTLTISSLQPEDFATYYCLQHDSFPITFGGGTKLEIK

SEQ ID NO: 261 - protein, 119 aa, synthetic construct
  REGION 1..119: note = Synthetic Construct
EVQLVQSGAEVKKPGASVKVSCKASGYTFTSYDINWVRQAPGQGLEWMGGIIPIFGTANYAQKFQGRVTITADESTSTAYMELRSLRSEDTAVYYCASTAVAGFGIDYWGQGTLVTVSS

SEQ ID NO: 262 - protein, 107 aa, synthetic construct
  REGION 1..107: note = Synthetic Construct
DIQMTQSPSTLSASVGDRVTITCRASQGISNYLAWYQQKPGKAPKLLIYAASSLQSGVPSRFSGSGSGTDFTLTISSLQPEDFATYYCQQSYSTPLTFGGGTKVEIK

SEQ ID NO: 263 - protein, 127 aa, synthetic construct
  REGION 1..127: note = Synthetic Construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAAGYYYDSSGYYHQPYYFDYWGQGTLVTVSS

SEQ ID NO: 264 - protein, 107 aa, synthetic construct
  REGION 1..107: note = Synthetic Construct
DIQMTQSPSSLSASVGDRVTITCQASQDISNYLNWYQQKPGKVPKLLIYAASTLQSGVPSRFSGSGSGTDFTLTISSLQPEDFATYYCQQSYSTPLTFGGGTKVEIK

SEQ ID NO: 265 - protein, 119 aa, synthetic construct
  REGION 1..119: note = Synthetic Construct
EVQLVESGGGLVQPGGSLRLSCAASGFTFSDYYMSWIRQAPGKGLEWVSGISWNSGSIGYADSVKGRFTISRDNSKNTLYLQMNSLRAEDTAVYYCASSGSWYLPLDYWGQGTLVTVSS

SEQ ID NO: 266 - protein, 107 aa, synthetic construct
  REGION 1..107: note = Synthetic Construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSLTFGGGTKVEIK

SEQ ID NO: 267 - protein, 121 aa, synthetic construct
  REGION 1..121: note = Synthetic Construct
EVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARATLQLWLPLFDYWGQGTLVTVSS

SEQ ID NO: 268 - protein, 107 aa, synthetic construct
  REGION 1..107: note = Synthetic Construct
DIVMTQSPSSLSASVGDRVTITCRASQGISNYLAWYQQKPGKVPKLLIYAASTLQSGVPSRFSGSGSGTEFTLAISSLQPEDLATYYCQQLNSHPITFGQGTRLEIK

SEQ ID NO: 269 - protein, 120 aa, synthetic construct
  REGION 1..120: note = Synthetic Construct
EVQLVQSGAEVKKPGSSVKVSCKASGGTFSSYAISWVRQAPGQGLEWMGWINPNSGGTNYAQKFQGRVTMTRDTSTSTAYMELSSLRSEDTAVYYCATGYYGDLYYFDYWGQGTLVTVSS

SEQ ID NO: 270 - protein, 107 aa, synthetic construct
  REGION 1..107: note = Synthetic Construct
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIK

SEQ ID NO: 271 - protein, 117 aa, synthetic construct
  REGION 1..117: note = Synthetic Construct
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 272 - protein, 11 aa, synthetic construct
  REGION 1..11: note = Synthetic Construct
RASQSISSYLN

SEQ ID NO: 273 - protein, 7 aa, synthetic construct
  REGION 1..7: note = Synthetic Construct
AASSLQS

SEQ ID NO: 274 - protein, 9 aa, synthetic construct
  REGION 1..9: note = Synthetic Construct
QQSYSTPLT

SEQ ID NO: 275 - protein, 7 aa, synthetic construct
  REGION 1..7: note = Synthetic Construct
GYTFTGY

SEQ ID NO: 276 - protein, 6 aa, synthetic construct
  REGION 1..6: note = Synthetic Construct
NPNSGG

SEQ ID NO: 277 - protein, 12 aa, synthetic construct
  REGION 1..12: note = Synthetic Construct
GSALHFFGVFDY

SEQ ID NO: 278 - protein, 11 aa, synthetic construct
  REGION 1..11: note = Synthetic Construct
RASQSISSYLN

SEQ ID NO: 279 - protein, 7 aa, synthetic construct
  REGION 1..7: note = Synthetic Construct
AASSLQS

SEQ ID NO: 280 - protein, 9 aa, synthetic construct
QQSYSTPLT

SEQ ID NO: 281 - protein, 5 aa, synthetic construct
  REGION 1..5: note = Synthetic Construct
GYYMH

SEQ ID NO: 282 - protein, 17 aa, synthetic construct
  REGION 1..17: note = Synthetic Construct
WINPNSGGTNYAQKFQG

SEQ ID NO: 283 - protein, 12 aa, synthetic construct
  REGION 1..12: note = Synthetic Construct
GSALHFFGVFDY

SEQ ID NO: 284 - protein, 6 aa, synthetic construct
  REGION 1..6: note = Synthetic Construct
QSISSY

SEQ ID NO: 285 - skipped

SEQ ID NO: 286 - protein, 9 aa, synthetic construct
  REGION 1..9: note = Synthetic Construct
QQSYSTPLT

SEQ ID NO: 287 - protein, 8 aa, synthetic construct
  REGION 1..8: note = Synthetic Construct
GYTFTGYY

SEQ ID NO: 288 - protein, 8 aa, synthetic construct
  REGION 1..8: note = Synthetic Construct
INPNSGGT

SEQ ID NO: 289 - protein, 14 aa, synthetic construct
  REGION 1..14: note = Synthetic Construct
ARGSALHFFGVFDY

SEQ ID NO: 290 - protein, 11 aa, synthetic construct
  REGION 1..11: note = Synthetic Construct
RASQGISNYLA

SEQ ID NO: 291 - protein, 7 aa, synthetic construct
  REGION 1..7: note = Synthetic Construct
AASTLQS

SEQ ID NO: 292 - protein, 9 aa, synthetic construct
  REGION 1..9: note = Synthetic Construct
QQSYSTPYT

SEQ ID NO: 293 - protein, 7 aa, synthetic construct
  REGION 1..7: note = Synthetic Construct
GGTFSSY

SEQ ID NO: 294 - protein, 6 aa, synthetic construct
  REGION 1..6: note = Synthetic Construct
IPIFGT

SEQ ID NO: 295 - protein, 14 aa, synthetic construct
  REGION 1..14: note = Synthetic Construct
GYSSSWYEPYYFDY

SEQ ID NO: 296 - protein, 11 aa, synthetic construct
  REGION 1..11: note = Synthetic Construct
RASQGISNYLA

SEQ ID NO: 297 - protein, 7 aa, synthetic construct
  REGION 1..7: note = Synthetic Construct
AASTLQS

SEQ ID NO: 298 - protein, 9 aa, synthetic construct
  REGION 1..9: note = Synthetic Construct
QQSYSTPYT

SEQ ID NO: 299 - protein, 5 aa, synthetic construct
  REGION 1..5: note = Synthetic Construct
SYAIS

SEQ ID NO: 300 - protein, 17 aa, synthetic construct
  REGION 1..17: note = Synthetic Construct
GIIPIFGTANYAQKFQG

SEQ ID NO: 301 - protein, 14 aa, synthetic construct
  REGION 1..14: note = Synthetic Construct
GYSSSWYEPYYFDY

SEQ ID NO: 302 - protein, 6 aa, synthetic construct
  REGION 1..6: note = Synthetic Construct
QGISNY

SEQ ID NO: 303 - skipped

SEQ ID NO: 304 - protein, 9 aa, synthetic construct
  REGION 1..9: note = Synthetic Construct
QQSYSTPYT

SEQ ID NO: 305 - protein, 8 aa, synthetic construct
  REGION 1..8: note = Synthetic Construct
GGTFSSYA

SEQ ID NO: 306 - protein, 8 aa, synthetic construct
  REGION 1..8: note = Synthetic Construct
IIPIFGTA

SEQ ID NO: 307 - protein, 16 aa, synthetic construct
  REGION 1..16: note = Synthetic Construct
ARGYSSSWYEPYYFDY

SEQ ID NO: 308 - protein, 11 aa, synthetic construct
  REGION 1..11: note = Synthetic Construct
RASQSVSSYLA

SEQ ID NO: 309 - protein, 7 aa, synthetic construct
  REGION 1..7: note = Synthetic Construct
DASNRAT

SEQ ID NO: 310 - protein, 7 aa, synthetic construct
  REGION 1..7: note = Synthetic Construct
QQSYSTR

SEQ ID NO: 311 - protein, 7 aa, synthetic construct
  REGION 1..7: note = Synthetic Construct
GFTFSDY

SEQ ID NO: 312 - protein, 6 aa, synthetic construct
  REGION 1..6: note = Synthetic Construct
SGSGGS

SEQ ID NO: 313 - protein, 9 aa, synthetic construct
  REGION 1..9: note = Synthetic Construct
DEGYYYMDV

SEQ ID NO: 314 - protein, 11 aa, synthetic construct
  REGION 1..11: note = Synthetic Construct
RASQSVSSYLA

SEQ ID NO: 315 - protein, 7 aa, synthetic construct
  REGION 1..7: note = Synthetic Construct
DASNRAT

SEQ ID NO: 316 - protein, 7 aa, synthetic construct
  REGION 1..7: note = Synthetic Construct
QQSYSTR

SEQ ID NO: 317 - protein, 5 aa, synthetic construct
  REGION 1..5: note = Synthetic Construct
DYYMS

SEQ ID NO: 318 - protein, 17 aa, synthetic construct
  REGION 1..17: note = Synthetic Construct
AISGSGGSTYYADSVKG

SEQ ID NO: 319 - protein, 9 aa, synthetic construct
  REGION 1..9: note = Synthetic Construct
DEGYYYMDV

SEQ ID NO: 320 - protein, 6 aa, synthetic construct
  REGION 1..6: note = Synthetic Construct
QSVSSY

SEQ ID NO: 321 - skipped

SEQ ID NO: 322 - protein, 7 aa, synthetic construct
  REGION 1..7: note = Synthetic Construct
QQSYSTR

SEQ ID NO: 323 - protein, 8 aa, synthetic construct
  REGION 1..8: note = Synthetic Construct
GFTFSDYY

SEQ ID NO: 324 - protein, 8 aa, synthetic construct
  REGION 1..8: note = Synthetic Construct
ISGSGGST

SEQ ID NO: 325 - protein, 11 aa, synthetic construct
  REGION 1..11: note = Synthetic Construct
ARDEGYYYMDV

SEQ ID NO: 326 - protein, 12 aa, synthetic construct
  REGION 1..12: note = Synthetic Construct
RASQSVSSSYLA

SEQ ID NO: 327 - protein, 7 aa, synthetic construct
  REGION 1..7: note = Synthetic Construct
GASSRAT

SEQ ID NO: 328 - protein, 8 aa, synthetic construct
  REGION 1..8: note = Synthetic Construct
QQYNNWWT

SEQ ID NO: 329 - protein, 7 aa, synthetic construct
  REGION 1..7: note = Synthetic Construct
GFTFSSY

SEQ ID NO: 330 - protein, 6 aa, synthetic construct
  REGION 1..6: note = Synthetic Construct
SYDGSN

SEQ ID NO: 331 - protein, 19 aa, synthetic construct
  REGION 1..19: note = Synthetic Construct
SSYYDYVWGSYRYTDAFDI

SEQ ID NO: 332 - protein, 12 aa, synthetic construct
  REGION 1..12: note = Synthetic Construct
RASQSVSSSYLA

SEQ ID NO: 333 - protein, 7 aa, synthetic construct
  REGION 1..7: note = Synthetic Construct
GASSRAT

SEQ ID NO: 334 - protein, 8 aa, synthetic construct
  REGION 1..8: note = Synthetic Construct
QQYNNWWT

SEQ ID NO: 335 - protein, 5 aa, synthetic construct
  REGION 1..5: note = Synthetic Construct
SYGMH

SEQ ID NO: 336 - protein, 17 aa, synthetic construct
  REGION 1..17: note = Synthetic Construct
VISYDGSNKYYADSVKG

SEQ ID NO: 337 - protein, 19 aa, synthetic construct
  REGION 1..19: note = Synthetic Construct
SSYYDYVWGSYRYTDAFDI

SEQ ID NO: 338 - protein, 7 aa, synthetic construct
  REGION 1..7: note = Synthetic Construct
QSVSSSY

SEQ ID NO: 339 - skipped

SEQ ID NO: 340 - protein, 8 aa, synthetic construct
  REGION 1..8: note = Synthetic Construct
QQYNNWWT

SEQ ID NO: 341 - protein, 8 aa, synthetic construct
  REGION 1..8: note = Synthetic Construct
GFTFSSYG

SEQ ID NO: 342 - protein, 8 aa, synthetic construct
  REGION 1..8: note = Synthetic Construct
ISYDGSNK

SEQ ID NO: 343 - protein, 21 aa, synthetic construct
  REGION 1..21: note = Synthetic Construct
AKSSYYDYVWGSYRYTDAFDI

SEQ ID NO: 344 - protein, 11 aa, synthetic construct
  REGION 1..11: note = Synthetic Construct
RASQGISNYLA

SEQ ID NO: 345 - protein, 7 aa, synthetic construct
  REGION 1..7: note = Synthetic Construct
AASTLQS

SEQ ID NO: 346 - protein, 9 aa, synthetic construct
  REGION 1..9: note = Synthetic Construct
QKYNSAPYT

SEQ ID NO: 347 - protein, 7 aa, synthetic construct
  REGION 1..7: note = Synthetic Construct
GFTFSSY

SEQ ID NO: 348 - protein, 6 aa, synthetic construct
  REGION 1..6: note = Synthetic Construct
SYDGSN

SEQ ID NO: 349 - protein, 11 aa, synthetic construct
  REGION 1..11: note = Synthetic Construct
DYGYNWNFFDY

SEQ ID NO: 350 - protein, 11 aa, synthetic construct
  REGION 1..11: note = Synthetic Construct
RASQGISNYLA

SEQ ID NO: 351 - protein, 7 aa, synthetic construct
  REGION 1..7: note = Synthetic Construct
AASTLQS

SEQ ID NO: 352 - protein, 9 aa, synthetic construct
  REGION 1..9: note = Synthetic Construct
QKYNSAPYT

SEQ ID NO: 353 - protein, 5 aa, synthetic construct
  REGION 1..5: note = Synthetic Construct
SYGMH

SEQ ID NO: 354 - protein, 17 aa, synthetic construct
  REGION 1..17: note = Synthetic Construct
VISYDGSNKYYADSVKG

SEQ ID NO: 355 - protein, 11 aa, synthetic construct
  REGION 1..11: note = Synthetic Construct
DYGYNWNFFDY

SEQ ID NO: 356 - protein, 6 aa, synthetic construct
  REGION 1..6: note = Synthetic Construct
QGISNY

SEQ ID NO: 357 - skipped

SEQ ID NO: 358 - protein, 9 aa, synthetic construct
  REGION 1..9: note = Synthetic Construct
QKYNSAPYT

SEQ ID NO: 359 - protein, 8 aa, synthetic construct
  REGION 1..8: note = Synthetic Construct
GFTFSSYG

SEQ ID NO: 360 - protein, 8 aa, synthetic construct
  REGION 1..8: note = Synthetic Construct
ISYDGSNK

SEQ ID NO: 361 - protein, 13 aa, synthetic construct
  REGION 1..13: note = Synthetic Construct
AKDYGYNWNFFDY

SEQ ID NO: 362 - protein, 11 aa, synthetic construct
  REGION 1..11: note = Synthetic Construct
RASQSVSSNLA

SEQ ID NO: 363 - protein, 7 aa, synthetic construct
  REGION 1..7: note = Synthetic Construct
GASSRAT

SEQ ID NO: 364 - protein, 9 aa, synthetic construct
  REGION 1..9: note = Synthetic Construct
HQYGSSPLT

SEQ ID NO: 365 - protein, 9 aa, synthetic construct
  REGION 1..9: note = Synthetic Construct
GGSISSGGY

SEQ ID NO: 366 - protein, 5 aa, synthetic construct
  REGION 1..5: note = Synthetic Construct
YYTGS

SEQ ID NO: 367 - protein, 12 aa, synthetic construct
  REGION 1..12: note = Synthetic Construct
DPFPEPPDAFDI

SEQ ID NO: 368 - protein, 11 aa, synthetic construct
  REGION 1..11: note = Synthetic Construct
RASQSVSSNLA

SEQ ID NO: 369 - protein, 7 aa, synthetic construct
  REGION 1..7: note = Synthetic Construct
GASSRAT

SEQ ID NO: 370 - protein, 9 aa, synthetic construct
  REGION 1..9: note = Synthetic Construct
HQYGSSPLT

SEQ ID NO: 371 - protein, 7 aa, synthetic construct
  REGION 1..7: note = Synthetic Construct
SGGYYWA

SEQ ID NO: 372 - protein, 16 aa, synthetic construct
  REGION 1..16: note = Synthetic Construct
SIYYTGSTYYNPSLKS

SEQ ID NO: 373 - protein, 12 aa, synthetic construct
  REGION 1..12: note = Synthetic Construct
DPFPEPPDAFDI

SEQ ID NO: 374 - protein, 6 aa, synthetic construct
  REGION 1..6: note = Synthetic Construct
QSVSSN

SEQ ID NO: 375 - skipped

SEQ ID NO: 376 - protein, 9 aa, synthetic construct
  REGION 1..9: note = Synthetic Construct
HQYGSSPLT

SEQ ID NO: 377 - protein, 10 aa, synthetic construct
  REGION 1..10: note = Synthetic Construct
GGSISSGGYY

SEQ ID NO: 378 - protein, 7 aa, synthetic construct
  REGION 1..7: note = Synthetic Construct
IYYTGST

SEQ ID NO: 379 - protein, 14 aa, synthetic construct
  REGION 1..14: note = Synthetic Construct
ARDPFPEPPDAFDI

SEQ ID NO: 380 - protein, 11 aa, synthetic construct
  REGION 1..11: note = Synthetic Construct
RASQGISNYLA

SEQ ID NO: 381 - protein, 7 aa, synthetic construct
  REGION 1..7: note = Synthetic Construct
AASSLQS

SEQ ID NO: 382 - protein, 9 aa, synthetic construct
  REGION 1..9: note = Synthetic Construct
LQHDSFPIT

SEQ ID NO: 383 - protein, 7 aa, synthetic construct
  REGION 1..7: note = Synthetic Construct
GYTFTSY

SEQ ID NO: 384 - protein, 6 aa, synthetic construct
  REGION 1..6: note = Synthetic Construct
IPIFGT

SEQ ID NO: 385 - protein, 10 aa, synthetic construct
  REGION 1..10: note = Synthetic Construct
TAVAGFGIDY

SEQ ID NO: 386 - protein, 11 aa, synthetic construct
  REGION 1..11: note = Synthetic Construct
RASQGISNYLA

SEQ ID NO: 387 - protein, 7 aa, synthetic construct
  REGION 1..7: note = Synthetic Construct
AASSLQS

SEQ ID NO: 388 - protein, 9 aa, synthetic construct
  REGION 1..9: note = Synthetic Construct
LQHDSFPIT

SEQ ID NO: 389 - protein, 5 aa, synthetic construct
  REGION 1..5: note = Synthetic Construct
SYDIN

SEQ ID NO: 390 - protein, 17 aa, synthetic construct
  REGION 1..17: note = Synthetic Construct
GIIPIFGTANYAQKFQG

SEQ ID NO: 391 - protein, 10 aa, synthetic construct
  REGION 1..10: note = Synthetic Construct
TAVAGFGIDY

SEQ ID NO: 392 - protein, 6 aa, synthetic construct
  REGION 1..6: note = Synthetic Construct
QGISNY

SEQ ID NO: 393 - skipped

SEQ ID NO: 394 - protein, 9 aa, synthetic construct
  REGION 1..9: note = Synthetic Construct
LQHDSFPIT

SEQ ID NO: 395 - protein, 8 aa, synthetic construct
  REGION 1..8: note = Synthetic Construct
GYTFTSYD

SEQ ID NO: 396 - protein, 8 aa, synthetic construct
  REGION 1..8: note = Synthetic Construct
IIPIFGTA

SEQ ID NO: 397 - protein, 12 aa, synthetic construct
  REGION 1..12: note = Synthetic Construct
ASTAVAGFGIDY

SEQ ID NO: 398 - protein, 11 aa, synthetic construct
  REGION 1..11: note = Synthetic Construct
RASQGISNYLA

SEQ ID NO: 399 - protein, 7 aa, synthetic construct
  REGION 1..7: note = Synthetic Construct
AASSLQS

SEQ ID NO: 400 - protein, 9 aa, synthetic construct
  REGION 1..9: note = Synthetic Construct
QQSYSTPLT

SEQ ID NO: 401 - protein, 7 aa, synthetic construct
  REGION 1..7: note = Synthetic Construct
GGTFSSY

SEQ ID NO: 402 - protein, 6 aa, synthetic construct
  REGION 1..6: note = Synthetic Construct
IPIFGT

SEQ ID NO: 403 - protein, 18 aa, synthetic construct
  REGION 1..18: note = Synthetic Construct
GYYYDSSGYYHQPYYFDY

SEQ ID NO: 404 - protein, 11 aa, synthetic construct
  REGION 1..11: note = Synthetic Construct
RASQGISNYLA

SEQ ID NO: 405 - protein, 7 aa, synthetic construct
  REGION 1..7: note = Synthetic Construct
AASSLQS

SEQ ID NO: 406 - protein, 9 aa, synthetic construct
  REGION 1..9: note = Synthetic Construct
QQSYSTPLT

SEQ ID NO: 407 - protein, 5 aa, synthetic construct
  REGION 1..5: note = Synthetic Construct
SYAIS

SEQ ID NO: 408 - protein, 17 aa, synthetic construct
  REGION 1..17: note = Synthetic Construct
GIIPIFGTANYAQKFQG

SEQ ID NO: 409 - protein, 18 aa, synthetic construct
  REGION 1..18: note = Synthetic Construct
GYYYDSSGYYHQPYYFDY

SEQ ID NO: 410 - protein, 6 aa, synthetic construct
  REGION 1..6: note = Synthetic Construct
QGISNY

SEQ ID NO: 411 - skipped

SEQ ID NO: 412 - protein, 9 aa, synthetic construct
  REGION 1..9: note = Synthetic Construct
QQSYSTPLT

SEQ ID NO: 413 - protein, 8 aa, synthetic construct
  REGION 1..8: note = Synthetic Construct
GGTFSSYA

SEQ ID NO: 414 - protein, 8 aa, synthetic construct
  REGION 1..8: note = Synthetic Construct
IIPIFGTA

SEQ ID NO: 415 - protein, 20 aa, synthetic construct
  REGION 1..20: note = Synthetic Construct
AAGYYYDSSGYYHQPYYFDY

SEQ ID NO: 416 - protein, 11 aa, synthetic construct
  REGION 1..11: note = Synthetic Construct
QASQDISNYLN

SEQ ID NO: 417 - protein, 7 aa, synthetic construct
  REGION 1..7: note = Synthetic Construct
AASTLQS

SEQ ID NO: 418 - protein, 9 aa, synthetic construct
  REGION 1..9: note = Synthetic Construct
QQSYSTPLT

SEQ ID NO: 419 - protein, 7 aa, synthetic construct
  REGION 1..7: note = Synthetic Construct
GFTFSDY

SEQ ID NO: 420 - protein, 6 aa, synthetic construct
  REGION 1..6: note = Synthetic Construct
SWNSGS

SEQ ID NO: 421 - protein, 10 aa, synthetic construct
  REGION 1..10: note = Synthetic Construct
SGSWYLPLDY

SEQ ID NO: 422 - protein, 11 aa, synthetic construct
  REGION 1..11: note = Synthetic Construct
QASQDISNYLN

SEQ ID NO: 423 - protein, 7 aa, synthetic construct
  REGION 1..7: note = Synthetic Construct
AASTLQS

SEQ ID NO: 424 - protein, 9 aa, synthetic construct
  REGION 1..9: note = Synthetic Construct
QQSYSTPLT

SEQ ID NO: 425 - protein, 5 aa, synthetic construct
  REGION 1..5: note = Synthetic Construct
DYYMS

SEQ ID NO: 426 - protein, 17 aa, synthetic construct
  REGION 1..17: note = Synthetic Construct
GISWNSGSIGYADSVKG

SEQ ID NO: 427 - protein, 10 aa, synthetic construct
  REGION 1..10: note = Synthetic Construct
SGSWYLPLDY

SEQ ID NO: 428 - protein, 6 aa, synthetic construct
  REGION 1..6: note = Synthetic Construct
QDISNY

SEQ ID NO: 429 - skipped

SEQ ID NO: 430 - protein, 9 aa, synthetic construct
  REGION 1..9: note = Synthetic Construct
QQSYSTPLT

SEQ ID NO: 431 - protein, 8 aa, synthetic construct
  REGION 1..8: note = Synthetic Construct
GFTFSDYY

SEQ ID NO: 432 - protein, 8 aa, synthetic construct
  REGION 1..8: note = Synthetic Construct
ISWNSGSI

SEQ ID NO: 433 - protein, 12 aa, synthetic construct
  REGION 1..12: note = Synthetic Construct
ASSGSWYLPLDY

SEQ ID NO: 434 - protein, 12 aa, synthetic construct
  REGION 1..12: note = Synthetic Construct
RASQSVSSSYLA

SEQ ID NO: 435 - protein, 7 aa, synthetic construct
  REGION 1..7: note = Synthetic Construct
GASSRAT

SEQ ID NO: 436 - protein, 8 aa, synthetic construct
  REGION 1..8: note = Synthetic Construct
QQYGSSLT

SEQ ID NO: 437 - protein, 7 aa, synthetic construct
  REGION 1..7: note = Synthetic Construct
GFTFSSY

SEQ ID NO: 438 - protein, 6 aa, synthetic construct
  REGION 1..6: note = Synthetic Construct
SYDGSN

SEQ ID NO: 439 - protein, 12 aa, synthetic construct
  REGION 1..12: note = Synthetic Construct
ATLQLWLPLFDY

SEQ ID NO: 440 - protein, 12 aa, synthetic construct
  REGION 1..12: note = Synthetic Construct
RASQSVSSSYLA

SEQ ID NO: 441 - protein, 7 aa, synthetic construct
  REGION 1..7: note = Synthetic Construct
GASSRAT

SEQ ID NO: 442 - protein, 8 aa, synthetic construct
  REGION 1..8: note = Synthetic Construct
QQYGSSLT

SEQ ID NO: 443 - protein, 5 aa, synthetic construct
  REGION 1..5: note = Synthetic Construct
SYAMH

SEQ ID NO: 444 - protein, 17 aa, synthetic construct
  REGION 1..17: note = Synthetic Construct
VISYDGSNKYYADSVKG

SEQ ID NO: 445 - protein, 12 aa, synthetic construct
  REGION 1..12: note = Synthetic Construct
ATLQLWLPLFDY

SEQ ID NO: 446 - protein, 7 aa, synthetic construct
  REGION 1..7: note = Synthetic Construct
QSVSSSY

SEQ ID NO: 447 - skipped

SEQ ID NO: 448 - protein, 8 aa, synthetic construct
  REGION 1..8: note = Synthetic Construct
QQYGSSLT

SEQ ID NO: 449 - protein, 8 aa, synthetic construct
  REGION 1..8: note = Synthetic Construct
GFTFSSYA

SEQ ID NO: 450 - protein, 8 aa, synthetic construct
  REGION 1..8: note = Synthetic Construct
ISYDGSNK

SEQ ID NO: 451 - protein, 14 aa, synthetic construct
  REGION 1..14: note = Synthetic Construct
ARATLQLWLPLFDY

SEQ ID NO: 452 - protein, 11 aa, synthetic construct
  REGION 1..11: note = Synthetic Construct
RASQGISNYLA

SEQ ID NO: 453 - protein, 7 aa, synthetic construct
  REGION 1..7: note = Synthetic Construct
AASTLQS

SEQ ID NO: 454 - protein, 9 aa, synthetic construct
  REGION 1..9: note = Synthetic Construct
QQLNSHPIT

SEQ ID NO: 455 - protein, 7 aa, synthetic construct
  REGION 1..7: note = Synthetic Construct
GGTFSSY

SEQ ID NO: 456 - protein, 6 aa, synthetic construct
  REGION 1..6: note = Synthetic Construct
NPNSGG

SEQ ID NO: 457 - protein, 11 aa, synthetic construct
  REGION 1..11: note = Synthetic Construct
GYYGDLYYFDY

SEQ ID NO: 458 - protein, 11 aa, synthetic construct
  REGION 1..11: note = Synthetic Construct
RASQGISNYLA

SEQ ID NO: 459 - protein, 7 aa, synthetic construct
  REGION 1..7: note = Synthetic Construct
AASTLQS

SEQ ID NO: 460 - protein, 9 aa, synthetic construct
  REGION 1..9: note = Synthetic Construct
QQLNSHPIT

SEQ ID NO: 461 - protein, 5 aa, synthetic construct
  REGION 1..5: note = Synthetic Construct
SYAIS

SEQ ID NO: 462 - protein, 17 aa, synthetic construct
  REGION 1..17: note = Synthetic Construct
WINPNSGGTNYAQKFQG

SEQ ID NO: 463 - protein, 11 aa, synthetic construct
  REGION 1..11: note = Synthetic Construct
GYYGDLYYFDY

SEQ ID NO: 464 - protein, 6 aa, synthetic construct
  REGION 1..6: note = Synthetic Construct
QGISNY

SEQ ID NO: 465 - skipped

SEQ ID NO: 466 - protein, 9 aa, synthetic construct
  REGION 1..9: note = Synthetic Construct
QQLNSHPIT

SEQ ID NO: 467 - protein, 8 aa, synthetic construct
  REGION 1..8: note = Synthetic Construct
GGTFSSYA

SEQ ID NO: 468 - protein, 8 aa, synthetic construct
  REGION 1..8: note = Synthetic Construct
INPNSGGT

SEQ ID NO: 469 - protein, 13 aa, synthetic construct
  REGION 1..13: note = Synthetic Construct
ATGYYGDLYYFDY

SEQ ID NO: 470 - protein, 11 aa, synthetic construct
  REGION 1..11: note = Synthetic Construct
RASQGISGWLA

SEQ ID NO: 471 - protein, 7 aa, synthetic construct
  REGION 1..7: note = Synthetic Construct
AASTLQS

SEQ ID NO: 472 - protein, 9 aa, synthetic construct
  REGION 1..9: note = Synthetic Construct
QQANSFPPT

SEQ ID NO: 473 - protein, 7 aa, synthetic construct
  REGION 1..7: note = Synthetic Construct
GGTFSSY

SEQ ID NO: 474 - protein, 6 aa, synthetic construct
  REGION 1..6: note = Synthetic Construct
SSSSST

SEQ ID NO: 475 - protein, 8 aa, synthetic construct
  REGION 1..8: note = Synthetic Construct
AYYYGMDV

SEQ ID NO: 476 - protein, 11 aa, synthetic construct
  REGION 1..11: note = Synthetic Construct
RASQGISGWLA

SEQ ID NO: 477 - protein, 7 aa, synthetic construct
  REGION 1..7: note = Synthetic Construct
AASTLQS

SEQ ID NO: 478 - protein, 9 aa, synthetic construct
  REGION 1..9: note = Synthetic Construct
QQANSFPPT

SEQ ID NO: 479 - protein, 5 aa, synthetic construct
  REGION 1..5: note = Synthetic Construct
SYNMN

SEQ ID NO: 480 - protein, 17 aa, synthetic construct
  REGION 1..17: note = Synthetic Construct
YISSSSSTIYYADSVKG

SEQ ID NO: 481 - protein, 8 aa, synthetic construct
  REGION 1..8: note = Synthetic Construct
AYYYGMDV

SEQ ID NO: 482 - protein, 6 aa, synthetic construct
  REGION 1..6: note = Synthetic Construct
QGISGW

SEQ ID NO: 483 - skipped

SEQ ID NO: 484 - protein, 9 aa, synthetic construct
  REGION 1..9: note = Synthetic Construct
QQANSFPPT

SEQ ID NO: 485 - protein, 8 aa, synthetic construct
  REGION 1..8: note = Synthetic Construct
GFTFSSYN

SEQ ID NO: 486 - protein, 8 aa, synthetic construct
  REGION 1..8: note = Synthetic Construct
ISSSSSTI

SEQ ID NO: 487 - protein, 10 aa, synthetic construct
  REGION 1..10: note = Synthetic Construct
ARAYYYGMDV

SEQ ID NO: 488 - protein, 339 aa, synthetic construct
  REGION 1..339: note = Synthetic Construct
GELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASHHHHHHGLNDIFEAQKIEWHE

SEQ ID NO: 489 - protein, 324 aa, synthetic construct
  REGION 1..324: note = Synthetic Construct
GELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASHHHHHH

SEQ ID NO: 490 - protein, 323 aa, synthetic construct
  REGION 1..323: note = Synthetic Construct
GELETSDVVTVVLGQDAKLPCFYRGDPDEQVGQVAWARVDPNEGIRELALLHSKYGLHVNPAYEDRVEQPPPPRDPLDGSVLLRNAVQADEGEYECRVSTFPAGSFQARMRLRVLVPPLPSLNPGPPLEEGQGLTLAASCTAEGSPAPSVTWDTEVKGTQSSRSFTHPRSAAVTSEFHLVPSRSMNGQPLTCVVSHPGLLQDRRITHTLQVAFLAEASVRGLEDQNLWQVGREGATLKCLSEGQPPPKYNWTRLDGPLPSGVRVKGDTLGFPPLTTEHSGVYVCHVSNELSSRDSQVTVEVLDPEDPGKQVDLVSASHHHHHH

SEQ ID NO: 491 - protein, 122 aa, synthetic construct
  REGION 1..122: note = Synthetic Construct
GELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVHHHHHH

SEQ ID NO: 492 - protein, 334 aa, synthetic construct
  REGION 1..334: note = Synthetic Construct
QVVQVNDSMYGFIGTDVVLHCSFANPLPSVKITQVTWQKSTNGSKQNVAIYNPSMGVSVLAPYRERVEFLRPSFTDGTIRLSRLELEDEGVYICEFATFPTGNRESQLNLTVMAKPTNWIEGTQAVLRAKKGQDDKVLVATCTSANGKPPSVVSWETRLKGEAEYQEIRNPNGTVTVISRYRLVPSREAHQQSLACIVNYHMDRFKESLTLNVQYEPEVTIEGFDGNWYLQRMDVKLTCKADANPPATEYHWTTLNGSLPKGVEAQNRTLFFKGPINYSLAGTYICEATNPIGTRSGQVEVNITEFPYTPSPPEHGRRAGPVPTAIIGHHHHHH

SEQ ID NO: 493 - protein, 324 aa, synthetic construct
  REGION 1..324: note = Synthetic Construct
GELETSDVVTVVLGQDAKLPCFYRGDSGEQVGQVAWARADAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHVGREGAMLKCLSEGQPPPSYNWTRLDGPLPSGVRVDGDTLGFPPLTTEHSGIYVCHVSNEFSSRDSQVTVDVLDPQEDSGKQVDLVSASHHHHHH

SEQ ID NO: 494 - protein, 329 aa, synthetic construct
  REGION 1..329: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPG

SEQ ID NO: 495 - protein, 329 aa, synthetic construct
  REGION 1..329: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 496 - protein, 329 aa, synthetic construct
  REGION 1..329: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 497 - protein, 329 aa, synthetic construct
  REGION 1..329: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKIKDTLMISRTPEVTCVVVDVSHEDPEVKFNWYVDGVEVHNAKTKPREEQYNSTYRVVSVLTVLHQDWLNGKEYKCKVSNKALPAPIEKTISKQKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 498 - skipped

SEQ ID NO: 499 - DNA, 14 bp, synthetic construct
  misc_feature 1..14: note = Synthetic Construct
  modified_base 2: mod_base = OTHER; note = iodo-uridine
  modified_base 1..5: mod_base = OTHER; note = phosphorothioate linkages
  modified_base 5..6: mod_base = OTHER; note = phosphotriester linker
  modified_base 6..14: mod_base = OTHER; note = phosphorothioate linkages
  modified_base 1..14: mod_base = OTHER; note = 2'-deoxynucleotides
  misc_feature 14: note = 3' spacer C3
  source 1..14: mol_type = other DNA
tncgtcgtgacgtt

SEQ ID NO: 500 - skipped

SEQ ID NO: 501 - protein, 338 aa, synthetic construct
  REGION 1..338: note = Synthetic Construct
GELETSDVVTVVLGQDAKLPCFYRGDPDEQVGQVAWARVDPNEGIRELALLHSKYGLHVNPAYEDRVEQPPPPRDPLDGSVLLRNAVQADEGEYECRVSTFPAGSFQARMRLRVLVPPLPSLNPGPPLEEGQGLTLAASCTAEGSPAPSVTWDTEVKGTQSSRSFTHPRSAAVTSEFHLVPSRSMNGQPLTCVVSHPGLLQDRRITHTLQVAFLAEASVRGLEDQNLWQVGREGATLKCLSEGQPPPKYNWTRLDGPLPSGVRVKGDTLGFPPLTTEHSGVYVCHVSNELSSRDSQVTVEVLDPEDPGKQVDLVSASHHHHHHGLNDIFEAQKIEWHE

SEQ ID NO: 502-549 - skipped (48 entries)

SEQ ID NO: 550 - protein, 13 aa, synthetic construct
  REGION 1..13: note = Synthetic Construct
SGSSSNIGNNYVS

SEQ ID NO: 551 - protein, 7 aa, synthetic construct
  REGION 1..7: note = Synthetic Construct
DNNKRPS

SEQ ID NO: 552 - protein, 12 aa, synthetic construct
  REGION 1..12: note = Synthetic Construct
GTWDSSLNAGWV

SEQ ID NO: 553 - protein, 7 aa, synthetic construct
  REGION 1..7: note = Synthetic Construct
GGSISSY

SEQ ID NO: 554 - protein, 5 aa, synthetic construct
  REGION 1..5: note = Synthetic Construct
YYSGN

SEQ ID NO: 555 - protein, 16 aa, synthetic construct
  REGION 1..16: note = Synthetic Construct
DFHHYDGSAYRWYFDL

SEQ ID NO: 556 - protein, 13 aa, synthetic construct
  REGION 1..13: note = Synthetic Construct
SGSSSNIGNNYVS

SEQ ID NO: 557 - protein, 7 aa, synthetic construct
  REGION 1..7: note = Synthetic Construct
DNNKRPS

SEQ ID NO: 558 - protein, 12 aa, synthetic construct
  REGION 1..12: note = Synthetic Construct
GTWDSSLNAGWV

SEQ ID NO: 559 - protein, 5 aa, synthetic construct
  REGION 1..5: note = Synthetic Construct
SYFWS

SEQ ID NO: 560 - protein, 16 aa, synthetic construct
  REGION 1..16: note = Synthetic Construct
YIYYSGNTNYNPSLKS

SEQ ID NO: 561 - protein, 16 aa, synthetic construct
  REGION 1..16: note = Synthetic Construct
DFHHYDGSAYRWYFDL

SEQ ID NO: 562 - protein, 8 aa, synthetic construct
  REGION 1..8: note = Synthetic Construct
SSNIGNNY

SEQ ID NO: 563 - skipped

SEQ ID NO: 564 - protein, 12 aa, synthetic construct
  REGION 1..12: note = Synthetic Construct
GTWDSSLNAGWV

SEQ ID NO: 565 - protein, 8 aa, synthetic construct
  REGION 1..8: note = Synthetic Construct
GGSISSYF

SEQ ID NO: 566 - protein, 7 aa, synthetic construct
  REGION 1..7: note = Synthetic Construct
IYYSGNT

SEQ ID NO: 567 - protein, 18 aa, synthetic construct
  REGION 1..18: note = Synthetic Construct
ARDFHHYDGSAYRWYFDL

SEQ ID NO: 568 - protein, 14 aa, synthetic construct
  REGION 1..14: note = Synthetic Construct
TLRSGINVGSYRIY

SEQ ID NO: 569 - protein, 11 aa, synthetic construct
  REGION 1..11: note = Synthetic Construct
YKSDSDKQQGS

SEQ ID NO: 570 - protein, 9 aa, synthetic construct
  REGION 1..9: note = Synthetic Construct
MIWHSSASV

SEQ ID NO: 571 - protein, 7 aa, synthetic construct
  REGION 1..7: note = Synthetic Construct
GGSISSN

SEQ ID NO: 572 - protein, 5 aa, synthetic construct
  REGION 1..5: note = Synthetic Construct
HISES

SEQ ID NO: 573 - protein, 12 aa, synthetic construct
  REGION 1..12: note = Synthetic Construct
EGEWKVLGAFDI

SEQ ID NO: 574 - protein, 14 aa, synthetic construct
  REGION 1..14: note = Synthetic Construct
TLRSGINVGSYRIY

SEQ ID NO: 575 - protein, 7 aa, synthetic construct
  REGION 1..7: note = Synthetic Construct
SDKQQGS

SEQ ID NO: 576 - protein, 9 aa, synthetic construct
  REGION 1..9: note = Synthetic Construct
MIWHSSASV

SEQ ID NO: 577 - protein, 5 aa, synthetic construct
  REGION 1..5: note = Synthetic Construct
SNYWT

SEQ ID NO: 578 - protein, 16 aa, synthetic construct
  REGION 1..16: note = Synthetic Construct
RIHISESTNYNPSLKS

SEQ ID NO: 579 - protein, 12 aa, synthetic construct
  REGION 1..12: note = Synthetic Construct
EGEWKVLGAFDI

SEQ ID NO: 580 - protein, 9 aa, synthetic construct
  REGION 1..9: note = Synthetic Construct
SGINVGSYR

SEQ ID NO: 581 - protein, 7 aa, synthetic construct
  REGION 1..7: note = Synthetic Construct
YKSDSDK

SEQ ID NO: 582 - protein, 9 aa, synthetic construct
  REGION 1..9: note = Synthetic Construct
MIWHSSASV

SEQ ID NO: 583 - protein, 8 aa, synthetic construct
  REGION 1..8: note = Synthetic Construct
GGSISSNY

SEQ ID NO: 584 - protein, 7 aa, synthetic construct
  REGION 1..7: note = Synthetic Construct
IHISEST

SEQ ID NO: 585 - protein, 14 aa, synthetic construct
  REGION 1..14: note = Synthetic Construct
AREGEWKVLGAFDI

SEQ ID NO: 586 - protein, 13 aa, synthetic construct
  REGION 1..13: note = Synthetic Construct
SGSSSNIGSNTVH

SEQ ID NO: 587 - protein, 7 aa, synthetic construct
  REGION 1..7: note = Synthetic Construct
GNYQRPS

SEQ ID NO: 588 - protein, 11 aa, synthetic construct
  REGION 1..11: note = Synthetic Construct
ATWDDSLNGWV

SEQ ID NO: 589 - protein, 7 aa, synthetic construct
  REGION 1..7: note = Synthetic Construct
GYTFTVY

SEQ ID NO: 590 - protein, 6 aa, synthetic construct
  REGION 1..6: note = Synthetic Construct
SPNSGG

SEQ ID NO: 591 - protein, 12 aa, synthetic construct
  REGION 1..12: note = Synthetic Construct
QTVVTPNWGFDY

SEQ ID NO: 592 - protein, 13 aa, synthetic construct
  REGION 1..13: note = Synthetic Construct
SGSSSNIGSNTVH

SEQ ID NO: 593 - protein, 7 aa, synthetic construct
  REGION 1..7: note = Synthetic Construct
GNYQRPS

SEQ ID NO: 594 - protein, 11 aa, synthetic construct
  REGION 1..11: note = Synthetic Construct
ATWDDSLNGWV

SEQ ID NO: 595 - protein, 5 aa, synthetic construct
  REGION 1..5: note = Synthetic Construct
VYYIH

SEQ ID NO: 596 - protein, 17 aa, synthetic construct
  REGION 1..17: note = Synthetic Construct
WISPNSGGTNYAQKFQG

SEQ ID NO: 597 - protein, 12 aa, synthetic construct
  REGION 1..12: note = Synthetic Construct
QTVVTPNWGFDY

SEQ ID NO: 598 - protein, 8 aa, synthetic construct
  REGION 1..8: note = Synthetic Construct
SSNIGSNT

SEQ ID NO: 599 - skipped

SEQ ID NO: 600 - protein, 11 aa, synthetic construct
  REGION 1..11: note = Synthetic Construct
ATWDDSLNGWV

SEQ ID NO: 601 - protein, 8 aa, synthetic construct
  REGION 1..8: note = Synthetic Construct
GYTFTVYY

SEQ ID NO: 602 - protein, 8 aa, synthetic construct
  REGION 1..8: note = Synthetic Construct
ISPNSGGT

SEQ ID NO: 603 - protein, 14 aa, synthetic construct
  REGION 1..14: note = Synthetic Construct
ARQTVVTPNWGFDY

SEQ ID NO: 604 - protein, 11 aa, synthetic construct
  REGION 1..11: note = Synthetic Construct
RASQGISNYLA

SEQ ID NO: 605 - protein, 7 aa, synthetic construct
  REGION 1..7: note = Synthetic Construct
AASSLQS

SEQ ID NO: 606 - protein, 9 aa, synthetic construct
  REGION 1..9: note = Synthetic Construct
LQHSFYPLT

SEQ ID NO: 607 - protein, 7 aa, synthetic construct
  REGION 1..7: note = Synthetic Construct
GGSISSY

SEQ ID NO: 608 - protein, 5 aa, synthetic construct
  REGION 1..5: note = Synthetic Construct
YYSGN

SEQ ID NO: 609 - protein, 12 aa, synthetic construct
  REGION 1..12: note = Synthetic Construct
GGVGAQWDAFDI

SEQ ID NO: 610 - protein, 11 aa, synthetic construct
  REGION 1..11: note = Synthetic Construct
RASQGISNYLA

SEQ ID NO: 611 - protein, 7 aa, synthetic construct
  REGION 1..7: note = Synthetic Construct
AASSLQS

SEQ ID NO: 612 - protein, 9 aa, synthetic construct
  REGION 1..9: note = Synthetic Construct
LQHSFYPLT

SEQ ID NO: 613 - protein, 5 aa, synthetic construct
  REGION 1..5: note = Synthetic Construct
SYYWS

SEQ ID NO: 614 - protein, 16 aa, synthetic construct
  REGION 1..16: note = Synthetic Construct
YIYYSGNVNYNPSLKS

SEQ ID NO: 615 - protein, 12 aa, synthetic construct
  REGION 1..12: note = Synthetic Construct
GGVGAQWDAFDI

SEQ ID NO: 616 - protein, 6 aa, synthetic construct
  REGION 1..6: note = Synthetic Construct
QGISNY

SEQ ID NO: 617 - skipped

SEQ ID NO: 618 - protein, 9 aa, synthetic construct
  REGION 1..9: note = Synthetic Construct
LQHSFYPLT

SEQ ID NO: 619 - protein, 8 aa, synthetic construct
  REGION 1..8: note = Synthetic Construct
GGSISSYY

SEQ ID NO: 620 - protein, 7 aa, synthetic construct
  REGION 1..7: note = Synthetic Construct
IYYSGNV

SEQ ID NO: 621 - protein, 14 aa, synthetic construct
  REGION 1..14: note = Synthetic Construct
AIGGVGAQWDAFDI

SEQ ID NO: 622 - protein, 13 aa, synthetic construct
  REGION 1..13: note = Synthetic Construct
SGSSSNIGNNYVS

SEQ ID NO: 623 - protein, 7 aa, synthetic construct
  REGION 1..7: note = Synthetic Construct
DNNKRPS

SEQ ID NO: 624 - protein, 12 aa, synthetic construct
  REGION 1..12: note = Synthetic Construct
GTWDSSLSAGVV

SEQ ID NO: 625 - protein, 7 aa, synthetic construct
  REGION 1..7: note = Synthetic Construct
GGSISSY

SEQ ID NO: 626 - protein, 5 aa, synthetic construct
  REGION 1..5: note = Synthetic Construct
YYSGN

SEQ ID NO: 627 - protein, 11 aa, synthetic construct
  REGION 1..11: note = Synthetic Construct
SESYGYWFFYL

SEQ ID NO: 628 - protein, 13 aa, synthetic construct
  REGION 1..13: note = Synthetic Construct
SGSSSNIGNNYVS

SEQ ID NO: 629 - protein, 7 aa, synthetic construct
  REGION 1..7: note = Synthetic Construct
DNNKRPS

SEQ ID NO: 630 - protein, 12 aa, synthetic construct
  REGION 1..12: note = Synthetic Construct
GTWDSSLSAGVV

SEQ ID NO: 631 - protein, 5 aa, synthetic construct
  REGION 1..5: note = Synthetic Construct
SYYWN

SEQ ID NO: 632 - protein, 16 aa, synthetic construct
  REGION 1..16: note = Synthetic Construct
YIYYSGNTNYNPSLKS

SEQ ID NO: 633 - protein, 11 aa, synthetic construct
  REGION 1..11: note = Synthetic Construct
SESYGYWFFYL

SEQ ID NO: 634 - protein, 8 aa, synthetic construct
  REGION 1..8: note = Synthetic Construct
SSNIGNNY

SEQ ID NO: 635 - skipped

SEQ ID NO: 636 - protein, 12 aa, synthetic construct
  REGION 1..12: note = Synthetic Construct
GTWDSSLSAGVV

SEQ ID NO: 637 - protein, 8 aa, synthetic construct
  REGION 1..8: note = Synthetic Construct
GGSISSYY

SEQ ID NO: 638 - protein, 7 aa, synthetic construct
  REGION 1..7: note = Synthetic Construct
IYYSGNT

SEQ ID NO: 639 - protein, 13 aa, synthetic construct
  REGION 1..13: note = Synthetic Construct
ARSESYGYWFFYL

SEQ ID NO: 640 - protein, 16 aa, synthetic construct
  REGION 1..16: note = Synthetic Construct
RSSQSLAYSDGNTYLN

SEQ ID NO: 641 - protein, 7 aa, synthetic construct
  REGION 1..7: note = Synthetic Construct
KVSNRDS

SEQ ID NO: 642 - protein, 9 aa, synthetic construct
  REGION 1..9: note = Synthetic Construct
MQGTHWPLT

SEQ ID NO: 643 - protein, 7 aa, synthetic construct
  REGION 1..7: note = Synthetic Construct
GGSISSY

SEQ ID NO: 644 - protein, 5 aa, synthetic construct
  REGION 1..5: note = Synthetic Construct
YYIGT

SEQ ID NO: 645 - protein, 11 aa, synthetic construct
  REGION 1..11: note = Synthetic Construct
SGNYYNDAFDI

SEQ ID NO: 646 - protein, 16 aa, synthetic construct
  REGION 1..16: note = Synthetic Construct
RSSQSLAYSDGNTYLN

SEQ ID NO: 647 - protein, 7 aa, synthetic construct
  REGION 1..7: note = Synthetic Construct
KVSNRDS

SEQ ID NO: 648 - protein, 9 aa, synthetic construct
  REGION 1..9: note = Synthetic Construct
MQGTHWPLT

SEQ ID NO: 649 - protein, 5 aa, synthetic construct
  REGION 1..5: note = Synthetic Construct
SYFWN

SEQ ID NO: 650 - protein, 16 aa, synthetic construct
  REGION 1..16: note = Synthetic Construct
YLYYIGTTNYNPSLKS

SEQ ID NO: 651 - protein, 11 aa, synthetic construct
  REGION 1..11: note = Synthetic Construct
SGNYYNDAFDI

SEQ ID NO: 652 - protein, 11 aa, synthetic construct
  REGION 1..11: note = Synthetic Construct
QSLAYSDGNTY

SEQ ID NO: 653 - skipped

SEQ ID NO: 654 - protein, 9 aa, synthetic construct
  REGION 1..9: note = Synthetic Construct
MQGTHWPLT

SEQ ID NO: 655 - protein, 8 aa, synthetic construct
  REGION 1..8: note = Synthetic Construct
GGSISSYF

SEQ ID NO: 656 - protein, 7 aa, synthetic construct
  REGION 1..7: note = Synthetic Construct
LYYIGTT

SEQ ID NO: 657 - protein, 13 aa, synthetic construct
  REGION 1..13: note = Synthetic Construct
ARSGNYYNDAFDI

SEQ ID NO: 658 - protein, 14 aa, synthetic construct
  REGION 1..14: note = Synthetic Construct
ASSTGAVTSGYYPN

SEQ ID NO: 659 - protein, 7 aa, synthetic construct
  REGION 1..7: note = Synthetic Construct
STSNKHS

SEQ ID NO: 660 - protein, 9 aa, synthetic construct
  REGION 1..9: note = Synthetic Construct
LLYYDGAHL

SEQ ID NO: 661 - protein, 7 aa, synthetic construct
  REGION 1..7: note = Synthetic Construct
GYTFTGY

SEQ ID NO: 662 - protein, 6 aa, synthetic construct
  REGION 1..6: note = Synthetic Construct
NPNSGG

SEQ ID NO: 663 - protein, 11 aa, synthetic construct
  REGION 1..11: note = Synthetic Construct
DTFYYYYGMDV

SEQ ID NO: 664 - protein, 14 aa, synthetic construct
  REGION 1..14: note = Synthetic Construct
ASSTGAVTSGYYPN

SEQ ID NO: 665 - protein, 7 aa, synthetic construct
  REGION 1..7: note = Synthetic Construct
STSNKHS

SEQ ID NO: 666 - protein, 9 aa, synthetic construct
  REGION 1..9: note = Synthetic Construct
LLYYDGAHL

SEQ ID NO: 667 - protein, 5 aa, synthetic construct
  REGION 1..5: note = Synthetic Construct
GYFIH

SEQ ID NO: 668 - protein, 17 aa, synthetic construct
  REGION 1..17: note = Synthetic Construct
WINPNSGGTNYAQKFQG

SEQ ID NO: 669 - protein, 11 aa, synthetic construct
  REGION 1..11: note = Synthetic Construct
DTFYYYYGMDV

SEQ ID NO: 670 - protein, 9 aa, synthetic construct
  REGION 1..9: note = Synthetic Construct
TGAVTSGYY

SEQ ID NO: 671 - skipped

SEQ ID NO: 672 - protein, 9 aa, synthetic construct
  REGION 1..9: note = Synthetic Construct
LLYYDGAHL

SEQ ID NO: 673 - protein, 8 aa, synthetic construct
  REGION 1..8: note = Synthetic Construct
GYTFTGYF

SEQ ID NO: 674 - protein, 8 aa, synthetic construct
  REGION 1..8: note = Synthetic Construct
INPNSGGT

SEQ ID NO: 675 - protein, 13 aa, synthetic construct
  REGION 1..13: note = Synthetic Construct
ARDTFYYYYGMDV

SEQ ID NO: 676 - protein, 14 aa, synthetic construct
  REGION 1..14: note = Synthetic Construct
TGTTSDIGSYNLVS

SEQ ID NO: 677 - protein, 7 aa, synthetic construct
  REGION 1..7: note = Synthetic Construct
EVSMSPS

SEQ ID NO: 678 - protein, 10 aa, synthetic construct
  REGION 1..10: note = Synthetic Construct
SSYAGSSTVV

SEQ ID NO: 679 - protein, 7 aa, synthetic construct
  REGION 1..7: note = Synthetic Construct
GGSISDY

SEQ ID NO: 680 - protein, 5 aa, synthetic construct
  REGION 1..5: note = Synthetic Construct
YTTGG

SEQ ID NO: 681 - protein, 10 aa, synthetic construct
  REGION 1..10: note = Synthetic Construct
DRNWEWNFDL

SEQ ID NO: 682 - protein, 14 aa, synthetic construct
  REGION 1..14: note = Synthetic Construct
TGTTSDIGSYNLVS

SEQ ID NO: 683 - protein, 7 aa, synthetic construct
  REGION 1..7: note = Synthetic Construct
EVSMSPS

SEQ ID NO: 684 - protein, 10 aa, synthetic construct
  REGION 1..10: note = Synthetic Construct
SSYAGSSTVV

SEQ ID NO: 685 - protein, 5 aa, synthetic construct
  REGION 1..5: note = Synthetic Construct
DYYWI

SEQ ID NO: 686 - protein, 16 aa, synthetic construct
  REGION 1..16: note = Synthetic Construct
RIYTTGGAKYNPSLKS

SEQ ID NO: 687 - protein, 10 aa, synthetic construct
  REGION 1..10: note = Synthetic Construct
DRNWEWNFDL

SEQ ID NO: 688 - protein, 9 aa, synthetic construct
  REGION 1..9: note = Synthetic Construct
TSDIGSYNL

SEQ ID NO: 689 - skipped

SEQ ID NO: 690 - protein, 10 aa, synthetic construct
  REGION 1..10: note = Synthetic Construct
SSYAGSSTVV

SEQ ID NO: 691 - protein, 8 aa, synthetic construct
  REGION 1..8: note = Synthetic Construct
GGSISDYY

SEQ ID NO: 692 - protein, 7 aa, synthetic construct
  REGION 1..7: note = Synthetic Construct
IYTTGGA

SEQ ID NO: 693 - protein, 12 aa, synthetic construct
  REGION 1..12: note = Synthetic Construct
ARDRNWEWNFDL

SEQ ID NO: 694 - protein, 14 aa, synthetic construct
  REGION 1..14: note = Synthetic Construct
TGSSSNIGAGYNVH

SEQ ID NO: 695 - protein, 7 aa, synthetic construct
  REGION 1..7: note = Synthetic Construct
GNSNRPS

SEQ ID NO: 696 - protein, 12 aa, synthetic construct
  REGION 1..12: note = Synthetic Construct
QSYDSSLSGSAV

SEQ ID NO: 697 - protein, 7 aa, synthetic construct
  REGION 1..7: note = Synthetic Construct
GGSMSSY

SEQ ID NO: 698 - protein, 5 aa, synthetic construct
  REGION 1..5: note = Synthetic Construct
YYSGN

SEQ ID NO: 699 - protein, 10 aa, synthetic construct
  REGION 1..10: note = Synthetic Construct
SGNFYWNFDL

SEQ ID NO: 700 - protein, 14 aa, synthetic construct
  REGION 1..14: note = Synthetic Construct
TGSSSNIGAGYNVH

SEQ ID NO: 701 - protein, 7 aa, synthetic construct
  REGION 1..7: note = Synthetic Construct
GNSNRPS

SEQ ID NO: 702 - protein, 12 aa, synthetic construct
  REGION 1..12: note = Synthetic Construct
QSYDSSLSGSAV

SEQ ID NO: 703 - protein, 5 aa, synthetic construct
  REGION 1..5: note = Synthetic Construct
SYFWS

SEQ ID NO: 704 - protein, 16 aa, synthetic construct
  REGION 1..16: note = Synthetic Construct
YMYYSGNTNYNPSLKS

SEQ ID NO: 705 - protein, 10 aa, synthetic construct
  REGION 1..10: note = Synthetic Construct
SGNFYWNFDL

SEQ ID NO: 706 - protein, 9 aa, synthetic construct
  REGION 1..9: note = Synthetic Construct
SSNIGAGYN

SEQ ID NO: 707 - skipped

SEQ ID NO: 708 - protein, 12 aa, synthetic construct
  REGION 1..12: note = Synthetic Construct
QSYDSSLSGSAV

SEQ ID NO: 709 - protein, 8 aa, synthetic construct
  REGION 1..8: note = Synthetic Construct
GGSMSSYF

SEQ ID NO: 710 - protein, 7 aa, synthetic construct
  REGION 1..7: note = Synthetic Construct
MYYSGNT

SEQ ID NO: 711 - protein, 12 aa, synthetic construct
  REGION 1..12: note = Synthetic Construct
ARSGNFYWNFDL

SEQ ID NO: 712 - protein, 17 aa, synthetic construct
  REGION 1..17: note = Synthetic Construct
RSSQSLLDSDDGNTYLD

SEQ ID NO: 713 - protein, 7 aa, synthetic construct
  REGION 1..7: note = Synthetic Construct
TLSYRAS

SEQ ID NO: 714 - protein, 9 aa, synthetic construct
  REGION 1..9: note = Synthetic Construct
MQRIEFPYT

SEQ ID NO: 715 - protein, 7 aa, synthetic construct
  REGION 1..7: note = Synthetic Construct
GFTFSSY

SEQ ID NO: 716 - protein, 6 aa, synthetic construct
  REGION 1..6: note = Synthetic Construct
SISSSY

SEQ ID NO: 717 - protein, 10 aa, synthetic construct
  REGION 1..10: note = Synthetic Construct
DRGLDDAFDI

SEQ ID NO: 718 - protein, 17 aa, synthetic construct
  REGION 1..17: note = Synthetic Construct
RSSQSLLDSDDGNTYLD

SEQ ID NO: 719 - protein, 7 aa, synthetic construct
  REGION 1..7: note = Synthetic Construct
TLSYRAS

SEQ ID NO: 720 - protein, 9 aa, synthetic construct
  REGION 1..9: note = Synthetic Construct
MQRIEFPYT

SEQ ID NO: 721 - protein, 5 aa, synthetic construct
  REGION 1..5: note = Synthetic Construct
SYNMN

SEQ ID NO: 722 - protein, 17 aa, synthetic construct
  REGION 1..17: note = Synthetic Construct
SISISSSYIYYADSVKG

SEQ ID NO: 723 - protein, 10 aa, synthetic construct
  REGION 1..10: note = Synthetic Construct
DRGLDDAFDI

SEQ ID NO: 724 - protein, 12 aa, synthetic construct
  REGION 1..12: note = Synthetic Construct
QSLLDSDDGNTY

SEQ ID NO: 725 - skipped

SEQ ID NO: 726 - protein, 9 aa, synthetic construct
  REGION 1..9: note = Synthetic Construct
MQRIEFPYT

SEQ ID NO: 727 - protein, 8 aa, synthetic construct
  REGION 1..8: note = Synthetic Construct
GFTFSSYN

SEQ ID NO: 728 - protein, 8 aa, synthetic construct
  REGION 1..8: note = Synthetic Construct
ISISSSYI

SEQ ID NO: 729 - protein, 12 aa, synthetic construct
  REGION 1..12: note = Synthetic Construct
ARDRGLDDAFDI

SEQ ID NO: 730 - protein, 13 aa, synthetic construct
  REGION 1..13: note = Synthetic Construct
SGSSSNIGNNYVS

SEQ ID NO: 731 - protein, 7 aa, synthetic construct
  REGION 1..7: note = Synthetic Construct
DNNKRPS

SEQ ID NO: 732 - protein, 12 aa, synthetic construct
  REGION 1..12: note = Synthetic Construct
GTWDTSLSSGWL

SEQ ID NO: 733 - protein, 7 aa, synthetic construct
  REGION 1..7: note = Synthetic Construct
GGSIRSY

SEQ ID NO: 734 - protein, 5 aa, synthetic construct
  REGION 1..5: note = Synthetic Construct
YTSGS

SEQ ID NO: 735 - protein, 9 aa, synthetic construct
  REGION 1..9: note = Synthetic Construct
ENWGPSFDY

SEQ ID NO: 736 - protein, 13 aa, synthetic construct
  REGION 1..13: note = Synthetic Construct
SGSSSNIGNNYVS

SEQ ID NO: 737 - protein, 7 aa, synthetic construct
  REGION 1..7: note = Synthetic Construct
DNNKRPS

SEQ ID NO: 738 - protein, 12 aa, synthetic construct
  REGION 1..12: note = Synthetic Construct
GTWDTSLSSGWL

SEQ ID NO: 739 - protein, 5 aa, synthetic construct
  REGION 1..5: note = Synthetic Construct
SYYWS

SEQ ID NO: 740 - protein, 16 aa, synthetic construct
  REGION 1..16: note = Synthetic Construct
RIYTSGSTNYNPSLKS

SEQ ID NO: 741 - protein, 9 aa, synthetic construct
  REGION 1..9: note = Synthetic Construct
ENWGPSFDY

SEQ ID NO: 742 - protein, 8 aa, synthetic construct
  REGION 1..8: note = Synthetic Construct
SSNIGNNY

SEQ ID NO: 743 - skipped

SEQ ID NO: 744 - protein, 12 aa, synthetic construct
  REGION 1..12: note = Synthetic Construct
GTWDTSLSSGWL

SEQ ID NO: 745 - protein, 8 aa, synthetic construct
  REGION 1..8: note = Synthetic Construct
GGSIRSYY

SEQ ID NO: 746 - protein, 7 aa, synthetic construct
  REGION 1..7: note = Synthetic Construct
IYTSGST

SEQ ID NO: 747 - protein, 11 aa, synthetic construct
  REGION 1..11: note = Synthetic Construct
AKENWGPSFDY

SEQ ID NO: 748 - protein, 11 aa, synthetic construct
  REGION 1..11: note = Synthetic Construct
RASQGINSYLA

SEQ ID NO: 749 - protein, 7 aa, synthetic construct
  REGION 1..7: note = Synthetic Construct
SASTLQG

SEQ ID NO: 750 - protein, 9 aa, synthetic construct
  REGION 1..9: note = Synthetic Construct
QQLNSYPFT

SEQ ID NO: 751 - protein, 7 aa, synthetic construct
  REGION 1..7: note = Synthetic Construct
GYTFTNY

SEQ ID NO: 752 - protein, 6 aa, synthetic construct
  REGION 1..6: note = Synthetic Construct
NPTLDS

SEQ ID NO: 753 - protein, 9 aa, synthetic construct
  REGION 1..9: note = Synthetic Construct
SAGFDAFDI

SEQ ID NO: 754 - protein, 11 aa, synthetic construct
  REGION 1..11: note = Synthetic Construct
RASQGINSYLA

SEQ ID NO: 755 - protein, 7 aa, synthetic construct
  REGION 1..7: note = Synthetic Construct
SASTLQG

SEQ ID NO: 756 - protein, 9 aa, synthetic construct
  REGION 1..9: note = Synthetic Construct
QQLNSYPFT

SEQ ID NO: 757 - protein, 5 aa, synthetic construct
  REGION 1..5: note = Synthetic Construct
NYYIH

SEQ ID NO: 758 - protein, 17 aa, synthetic construct
  REGION 1..17: note = Synthetic Construct
IINPTLDSTSYAQKFQD

SEQ ID NO: 759 - protein, 9 aa, synthetic construct
  REGION 1..9: note = Synthetic Construct
SAGFDAFDI

SEQ ID NO: 760 - protein, 6 aa, synthetic construct
  REGION 1..6: note = Synthetic Construct
QGINSY

SEQ ID NO: 761 - skipped

SEQ ID NO: 762 - protein, 9 aa, synthetic construct
  REGION 1..9: note = Synthetic Construct
QQLNSYPFT

SEQ ID NO: 763 - protein, 8 aa, synthetic construct
  REGION 1..8: note = Synthetic Construct
GYTFTNYY

SEQ ID NO: 764 - protein, 8 aa, synthetic construct
  REGION 1..8: note = Synthetic Construct
INPTLDST

SEQ ID NO: 765 - protein, 11 aa, synthetic construct
  REGION 1..11: note = Synthetic Construct
ARSAGFDAFDI

SEQ ID NO: 766 - protein, 13 aa, synthetic construct
  REGION 1..13: note = Synthetic Construct
SGSSSNIGNNYVS

SEQ ID NO: 767 - protein, 7 aa, synthetic construct
  REGION 1..7: note = Synthetic Construct
DNNKRPS

SEQ ID NO: 768 - protein, 12 aa, synthetic construct
  REGION 1..12: note = Synthetic Construct
GTWDSSLSAGVV

SEQ ID NO: 769 - protein, 7 aa, synthetic construct
  REGION 1..7: note = Synthetic Construct
GGSISSY

SEQ ID NO: 770 - protein, 5 aa, synthetic construct
  REGION 1..5: note = Synthetic Construct
FYIGN

SEQ ID NO: 771 - protein, 9 aa, synthetic construct
  REGION 1..9: note = Synthetic Construct
SYPYWYFDL

SEQ ID NO: 772 - protein, 13 aa, synthetic construct
  REGION 1..13: note = Synthetic Construct
SGSSSNIGNNYVS

SEQ ID NO: 773 - protein, 7 aa, synthetic construct
  REGION 1..7: note = Synthetic Construct
DNNKRPS

SEQ ID NO: 774 - protein, 12 aa, synthetic construct
  REGION 1..12: note = Synthetic Construct
GTWDSSLSAGVV

SEQ ID NO: 775 - protein, 5 aa, synthetic construct
  REGION 1..5: note = Synthetic Construct
SYYWS

SEQ ID NO: 776 - protein, 16 aa, synthetic construct
  REGION 1..16: note = Synthetic Construct
YIFYIGNTNYYPSLKS

SEQ ID NO: 777 - protein, 9 aa, synthetic construct
  REGION 1..9: note = Synthetic Construct
SYPYWYFDL

SEQ ID NO: 778 - protein, 8 aa, synthetic construct
  REGION 1..8: note = Synthetic Construct
SSNIGNNY

SEQ ID NO: 779 - skipped

SEQ ID NO: 780 - protein, 12 aa, synthetic construct
  REGION 1..12: note = Synthetic Construct
GTWDSSLSAGVV

SEQ ID NO: 781 - protein, 8 aa, synthetic construct
  REGION 1..8: note = Synthetic Construct
GGSISSYY

SEQ ID NO: 782 - protein, 7 aa, synthetic construct
  REGION 1..7: note = Synthetic Construct
IFYIGNT

SEQ ID NO: 783 - protein, 11 aa, synthetic construct
  REGION 1..11: note = Synthetic Construct
ARSYPYWYFDL

SEQ ID NO: 784 - protein, 11 aa, synthetic construct
  REGION 1..11: note = Synthetic Construct
RASQSVSINLA

SEQ ID NO: 785 - protein, 7 aa, synthetic construct
  REGION 1..7: note = Synthetic Construct
GVSTRAT

SEQ ID NO: 786 - protein, 9 aa, synthetic construct
  REGION 1..9: note = Synthetic Construct
QQYNNWPRT

SEQ ID NO: 787 - protein, 7 aa, synthetic construct
  REGION 1..7: note = Synthetic Construct
GFTFSRY

SEQ ID NO: 788 - protein, 6 aa, synthetic construct
  REGION 1..6: note = Synthetic Construct
NSDGSS

SEQ ID NO: 789 - protein, 7 aa, synthetic construct
  REGION 1..7: note = Synthetic Construct
DNWYFDY

SEQ ID NO: 790 - protein, 11 aa, synthetic construct
  REGION 1..11: note = Synthetic Construct
RASQSVSINLA

SEQ ID NO: 791 - protein, 7 aa, synthetic construct
  REGION 1..7: note = Synthetic Construct
GVSTRAT

SEQ ID NO: 792 - protein, 9 aa, synthetic construct
  REGION 1..9: note = Synthetic Construct
QQYNNWPRT

SEQ ID NO: 793 - protein, 5 aa, synthetic construct
  REGION 1..5: note = Synthetic Construct
RYWMH

SEQ ID NO: 794 - protein, 17 aa, synthetic construct
  REGION 1..17: note = Synthetic Construct
RINSDGSSSNYADSVKG

SEQ ID NO: 795 - protein, 7 aa, synthetic construct
  REGION 1..7: note = Synthetic Construct
DNWYFDY

SEQ ID NO: 796 - protein, 6 aa, synthetic construct
  REGION 1..6: note = Synthetic Construct
QSVSIN

SEQ ID NO: 797 - skipped

SEQ ID NO: 798 - protein, 9 aa, synthetic construct
  REGION 1..9: note = Synthetic Construct
QQYNNWPRT

SEQ ID NO: 799 - protein, 8 aa, synthetic construct
  REGION 1..8: note = Synthetic Construct
GFTFSRYW

SEQ ID NO: 800 - protein, 8 aa, synthetic construct
  REGION 1..8: note = Synthetic Construct
INSDGSSS

SEQ ID NO: 801 - protein, 9 aa, synthetic construct
  REGION 1..9: note = Synthetic Construct
VRDNWYFDY

SEQ ID NO: 802 - protein, 13 aa, synthetic construct
  REGION 1..13: note = Synthetic Construct
SGSSSNIGSDYIY

SEQ ID NO: 803 - protein, 7 aa, synthetic construct
  REGION 1..7: note = Synthetic Construct
SNHQRPS

SEQ ID NO: 804 - protein, 11 aa, synthetic construct
  REGION 1..11: note = Synthetic Construct
AAWEDSLSGPI

SEQ ID NO: 805 - protein, 7 aa, synthetic construct
  REGION 1..7: note = Synthetic Construct
GGSISSY

SEQ ID NO: 806 - protein, 5 aa, synthetic construct
  REGION 1..5: note = Synthetic Construct
HYSGI

SEQ ID NO: 807 - protein, 7 aa, synthetic construct
  REGION 1..7: note = Synthetic Construct
GSYWFDP

SEQ ID NO: 808 - protein, 13 aa, synthetic construct
  REGION 1..13: note = Synthetic Construct
SGSSSNIGSDYIY

SEQ ID NO: 809 - protein, 7 aa, synthetic construct
  REGION 1..7: note = Synthetic Construct
SNHQRPS

SEQ ID NO: 810 - protein, 11 aa, synthetic construct
  REGION 1..11: note = Synthetic Construct
AAWEDSLSGPI

SEQ ID NO: 811 - protein, 5 aa, synthetic construct
  REGION 1..5: note = Synthetic Construct
SYYWS

SEQ ID NO: 812 - protein, 16 aa, synthetic construct
  REGION 1..16: note = Synthetic Construct
YIHYSGISNYNPSLKS

SEQ ID NO: 813 - protein, 7 aa, synthetic construct
  REGION 1..7: note = Synthetic Construct
GSYWFDP

SEQ ID NO: 814 - protein, 8 aa, synthetic construct
  REGION 1..8: note = Synthetic Construct
SSNIGSDY

SEQ ID NO: 815 - skipped

SEQ ID NO: 816 - protein, 11 aa, synthetic construct
  REGION 1..11: note = Synthetic Construct
AAWEDSLSGPI

SEQ ID NO: 817 - protein, 8 aa, synthetic construct
  REGION 1..8: note = Synthetic Construct
GGSISSYY

SEQ ID NO: 818 - protein, 7 aa, synthetic construct
  REGION 1..7: note = Synthetic Construct
IHYSGIS

SEQ ID NO: 819 - protein, 9 aa, synthetic construct
  REGION 1..9: note = Synthetic Construct
ARGSYWFDP

SEQ ID NO: 820 - protein, 12 aa, synthetic construct
  REGION 1..12: note = Synthetic Construct
TLSSGHSFYAIA

SEQ ID NO: 821 - protein, 11 aa, synthetic construct
  REGION 1..11: note = Synthetic Construct
VNSQGSHNKGD

SEQ ID NO: 822 - protein, 9 aa, synthetic construct
  REGION 1..9: note = Synthetic Construct
QSWDTDIWV

SEQ ID NO: 823 - protein, 7 aa, synthetic construct
  REGION 1..7: note = Synthetic Construct
GFTFSNY

SEQ ID NO: 824 - protein, 6 aa, synthetic construct
  REGION 1..6: note = Synthetic Construct
NNGGTS

SEQ ID NO: 825 - protein, 6 aa, synthetic construct
  REGION 1..6: note = Synthetic Construct
DWYFDL

SEQ ID NO: 826 - protein, 12 aa, synthetic construct
  REGION 1..12: note = Synthetic Construct
TLSSGHSFYAIA

SEQ ID NO: 827 - protein, 7 aa, synthetic construct
  REGION 1..7: note = Synthetic Construct
GSHNKGD

SEQ ID NO: 828 - protein, 9 aa, synthetic construct
  REGION 1..9: note = Synthetic Construct
QSWDTDIWV

SEQ ID NO: 829 - protein, 5 aa, synthetic construct
  REGION 1..5: note = Synthetic Construct
NYWMH

SEQ ID NO: 830 - protein, 17 aa, synthetic construct
  REGION 1..17: note = Synthetic Construct
RLNNGGTSTRYADSVKS

SEQ ID NO: 831 - protein, 6 aa, synthetic construct
  REGION 1..6: note = Synthetic Construct
DWYFDL

SEQ ID NO: 832 - protein, 7 aa, synthetic construct
  REGION 1..7: note = Synthetic Construct
SGHSFYA

SEQ ID NO: 833 - protein, 7 aa, synthetic construct
  REGION 1..7: note = Synthetic Construct
VNSQGSH

SEQ ID NO: 834 - protein, 9 aa, synthetic construct
  REGION 1..9: note = Synthetic Construct
QSWDTDIWV

SEQ ID NO: 835 - protein, 8 aa, synthetic construct
  REGION 1..8: note = Synthetic Construct
GFTFSNYW

SEQ ID NO: 836 - protein, 8 aa, synthetic construct
  REGION 1..8: note = Synthetic Construct
LNNGGTST

SEQ ID NO: 837 - protein, 8 aa, synthetic construct
  REGION 1..8: note = Synthetic Construct
VRDWYFDL

SEQ ID NO: 838 - protein, 16 aa, synthetic construct
  REGION 1..16: note = Synthetic Construct
RSSQSIVHSNANTYLE

SEQ ID NO: 839 - protein, 7 aa, synthetic construct
  REGION 1..7: note = Synthetic Construct
KVSNRFS

SEQ ID NO: 840 - protein, 9 aa, synthetic construct
  REGION 1..9: note = Synthetic Construct
FQGSHVPYT

SEQ ID NO: 841 - protein, 7 aa, synthetic construct
  REGION 1..7: note = Synthetic Construct
GFTFSNY

SEQ ID NO: 842 - protein, 6 aa, synthetic construct
  REGION 1..6: note = Synthetic Construct
SSGGSY

SEQ ID NO: 843 - protein, 11 aa, synthetic construct
  REGION 1..11: note = Synthetic Construct
QELGSYYAMDY

SEQ ID NO: 844 - protein, 16 aa, synthetic construct
  REGION 1..16: note = Synthetic Construct
RSSQSIVHSNANTYLE

SEQ ID NO: 845 - protein, 7 aa, synthetic construct
  REGION 1..7: note = Synthetic Construct
KVSNRFS

SEQ ID NO: 846 - protein, 9 aa, synthetic construct
  REGION 1..9: note = Synthetic Construct
FQGSHVPYT

SEQ ID NO: 847 - protein, 5 aa, synthetic construct
  REGION 1..5: note = Synthetic Construct
NYDMS

SEQ ID NO: 848 - protein, 17 aa, synthetic construct
  REGION 1..17: note = Synthetic Construct
TISSGGSYTYYVDSVKG

SEQ ID NO: 849 - protein, 11 aa, synthetic construct
  REGION 1..11: note = Synthetic Construct
QELGSYYAMDY

SEQ ID NO: 850 - protein, 11 aa, synthetic construct
  REGION 1..11: note = Synthetic Construct
QSIVHSNANTY

SEQ ID NO: 851 - skipped

SEQ ID NO: 852 - protein, 9 aa, synthetic construct
  REGION 1..9: note = Synthetic Construct
FQGSHVPYT

SEQ ID NO: 853 - protein, 8 aa, synthetic construct
  REGION 1..8: note = Synthetic Construct
GFTFSNYD

SEQ ID NO: 854 - protein, 8 aa, synthetic construct
  REGION 1..8: note = Synthetic Construct
ISSGGSYT

SEQ ID NO: 855 - protein, 13 aa, synthetic construct
  REGION 1..13: note = Synthetic Construct
ARQELGSYYAMDY

SEQ ID NO: 856-899 - skipped (44 entries)

SEQ ID NO: 900 - protein, 111 aa, synthetic construct
  REGION 1..111: note = Synthetic Construct
QSVLTQPPSVSAAPGQKVTISCSGSSSNIGNNYVSWYQQFPGTAPKLLIYDNNKRPSGIPDRFSGSKSGTSATLGITGLQTGDEADYYCGTWDSSLNAGWVFGGGTKLTVL

SEQ ID NO: 901 - protein, 124 aa, synthetic construct
  REGION 1..124: note = Synthetic Construct
QVQLQESGPGLVKPSETLSLTCTVSGGSISSYFWSWIRQPPGKGLEWIGYIYYSGNTNYNPSLKSRVTISVDMSKNQFSLRLSSVTAADTAVYYCARDFHHYDGSAYRWYFDLWGRGTLVTVSS

SEQ ID NO: 902 - protein, 115 aa, synthetic construct
  REGION 1..115: note = Synthetic Construct
QTVLTQPSSLSASPGASASLTCTLRSGINVGSYRIYWYQQKPGSPPQFLLRYKSDSDKQQGSGVPSRFSGSKDDSANAGILLISGLQSEDEADYYCMIWHSSASVFGTGTKVTVL

SEQ ID NO: 903 - protein, 120 aa, synthetic construct
  REGION 1..120: note = Synthetic Construct
QVQLQESGPGLVKPSETLSLTCTVSGGSISSNYWTWIRQSAGKGLEWIGRIHISESTNYNPSLKSRVTMSVDTSKNQFSLKLSSVTAADTAVYFCAREGEWKVLGAFDIWGQGTMVTVSS

SEQ ID NO: 904 - protein, 110 aa, synthetic construct
  REGION 1..110: note = Synthetic Construct
QSVLTQSPSASGTPGQRVTISCSGSSSNIGSNTVHWYQQLPGTAPKLLMYGNYQRPSGVPARFSGSKSGTSASLAISGLQSEDEADYYCATWDDSLNGWVFGGGTKVTVL

SEQ ID NO: 905 - protein, 121 aa, synthetic construct
  REGION 1..121: note = Synthetic Construct
QVQLVQSGAEVKKPGASVKVSCKASGYTFTVYYIHWVRQAPGQGLEWMGWISPNSGGTNYAQKFQGRVTMTRDTSISTAYMELTRLRSDDTAIFYCARQTVVTPNWGFDYWGQGTLVTVSS

SEQ ID NO: 906 - protein, 107 aa, synthetic construct
  REGION 1..107: note = Synthetic Construct
DIQMTQSPSAMSASVGDRVTITCRASQGISNYLAWFQQKPGKVPKRLIYAASSLQSGVPSRFSGSGSGTEFTLTISSLQPEDFATYYCLQHSFYPLTFGPGTKVDIK

SEQ ID NO: 907 - protein, 120 aa, synthetic construct
  REGION 1..120: note = Synthetic Construct
QVQLQESGPGLVKPSETLSLTCTVSGGSISSYYWSWIRQPPGKGLEWIGYIYYSGNVNYNPSLKSRVTISVDASKNQFSLKLTSVTAADTAVYYCAIGGVGAQWDAFDIWGQGTMVTVSS

SEQ ID NO: 908 - protein, 111 aa, synthetic construct
  REGION 1..111: note = Synthetic Construct
QSVLTQPPSVSAAPGQKVTISCSGSSSNIGNNYVSWYQQFPGTAPKLLIYDNNKRPSGIPDRFSGSKSGTSATLGITGLQTGDEADYYCGTWDSSLSAGVVFGGGTKLTVL

SEQ ID NO: 909 - protein, 119 aa, synthetic construct
  REGION 1..119: note = Synthetic Construct
QVQLQESGPGLVKPSATLSLTCTVSGGSISSYYWNWIRQPPGKGLEWIGYIYYSGNTNYNPSLKSRVTISVDMSKNQFSLKLNSVTAADTAVYYCARSESYGYWFFYLWGRGTLVTVSS

SEQ ID NO: 910 - protein, 112 aa, synthetic construct
  REGION 1..112: note = Synthetic Construct
DVVMTQSPLSLPVTLGQPASMSCRSSQSLAYSDGNTYLNWFQQRPGQSPRRLIYKVSNRDSGVPDRFSGSGSGTDFTLKISRVEAEDVGVYYCMQGTHWPLTFGGGTKVEIK

SEQ ID NO: 911 - protein, 119 aa, synthetic construct
  REGION 1..119: note = Synthetic Construct
QVQLQESGPGLVKPSETLSLTCTVSGGSISSYFWNWIRQPPGKGLEWIGYLYYIGTTNYNPSLKSRVTISVDTSKNQFSLKLNSVTAADTAVYYCARSGNYYNDAFDIWGQGTMVTVSS

SEQ ID NO: 912 - protein, 109 aa, synthetic construct
  REGION 1..109: note = Synthetic Construct
QTVVTQEPSLTVSPGGTVTLTCASSTGAVTSGYYPNWFQQKPGQAPRALIYSTSNKHSWTPARFSGSLLGGKAALTLSGVQPEDEAEYYCLLYYDGAHLFGGGTKLTVL

SEQ ID NO: 913 - protein, 120 aa, synthetic construct
  REGION 1..120: note = Synthetic Construct
QVQLVQSGAEVKKPGASVKVSCKASGYTFTGYFIHWVRQAPGQGLEWMGWINPNSGGTNYAQKFQGRVTMTRDTSISTAYMELSRLRSDDTAVYYCARDTFYYYYGMDVWGQGTTVTVSS

SEQ ID NO: 914 - protein, 110 aa, synthetic construct
  REGION 1..110: note = Synthetic Construct
QSALTQPASVSGSPGQSITISCTGTTSDIGSYNLVSWYQQHPGKAPKLMIYEVSMSPSGVSDRFSGSKSGSTASLTISGLQAEDEADYYCSSYAGSSTVVFGGGTRLTVL

SEQ ID NO: 915 - protein, 118 aa, synthetic construct
  REGION 1..118: note = Synthetic Construct
QVQLQESGPGLVKPSETLSLTCTVSGGSISDYYWIWIRQPAGKGLEWIGRIYTTGGAKYNPSLKSRVTMSIDTSKNNFSLKLSSVTAADTAVYFCARDRNWEWNFDLWGRGTLVTVSS

SEQ ID NO: 916 - protein, 112 aa, synthetic construct
  REGION 1..112: note = Synthetic Construct
QSVLTQPPSVSGAPGQRVTISCTGSSSNIGAGYNVHWYQQLPGTAPKVLISGNSNRPSGVPDRFSGSKSGTSASLAITGLQAEDEADYYCQSYDSSLSGSAVFGGGTKLTVL

SEQ ID NO: 917 - protein, 118 aa, synthetic construct
  REGION 1..118: note = Synthetic Construct
QVQLQESGPGLVKPSETLSLTCTVSGGSMSSYFWSWIRQPPERGLEWVGYMYYSGNTNYNPSLKSRVTTSIDTSKNQFSLKLTSVTAADTAVYYCARSGNFYWNFDLWGRGTLVTVSS

SEQ ID NO: 918 - protein, 113 aa, synthetic construct
  REGION 1..113: note = Synthetic Construct
DIVMTQTPLSLPVTPGEPASISCRSSQSLLDSDDGNTYLDWYLQKPGQSPQLLIYTLSYRASGVPDRFSGSGSGTDFTLKVSRVEAEDVGVYYCMQRIEFPYTFGQGTKLEIK

SEQ ID NO: 919 - protein, 119 aa, synthetic construct
  REGION 1..119: note = Synthetic Construct
EVQLVESGGGLVKPGGSLRLSCAVSGFTFSSYNMNWVRQAPGKGLEWVSSISISSSYIYYADSVKGRFTISRDNAKNSLYLQMNSLRAEDTAVYYCARDRGLDDAFDIWGQGTVVTVSS

SEQ ID NO: 920 - protein, 111 aa, synthetic construct
  REGION 1..111: note = Synthetic Construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDTSLSSGWLFGGGTKLTVL

SEQ ID NO: 921 - protein, 117 aa, synthetic construct
  REGION 1..117: note = Synthetic Construct
QVHLQESGPGLVKPSETLSLTCTVSGGSIRSYYWSWIRQPAGKGLEWIGRIYTSGSTNYNPSLKSRVTMSVDTSKNRLSLKLSSVTAADTAVYYCAKENWGPSFDYWGQGTLVTVSS

SEQ ID NO: 922 - protein, 107 aa, synthetic construct
  REGION 1..107: note = Synthetic Construct
DIQLTQSPSFLSASVGDKVTITCRASQGINSYLAWYQQKPGKAPKLLIYSASTLQGGVPSRFSGSGSGTEFTLTISNLQPADFATYCCQQLNSYPFTFGPGTKVDIK

SEQ ID NO: 923 - protein, 118 aa, synthetic construct
  REGION 1..118: note = Synthetic Construct
QVQLVQSGAEAKKPGASVRVSCKTSGYTFTNYYIHWVRQAPGQGLEWMGIINPTLDSTSYAQKFQDRVTMTRDTSTNTDYMDLSSLRFEDTAVYYCARSAGFDAFDIWGQGTMVTVSS

SEQ ID NO: 924 - protein, 111 aa, synthetic construct
  REGION 1..111: note = Synthetic Construct
QSVLTQPPSVSAAPGQKVTISCSGSSSNIGNNYVSWYQQLPGTAPKLLISDNNKRPSGIPDRFSGSKSGTSATLGITGLQTGDEADYYCGTWDSSLSAGVVFGGGTKLTVL

SEQ ID NO: 925 - protein, 117 aa, synthetic construct
  REGION 1..117: note = Synthetic Construct
QVQLQESGPGLVKPSETLSLTCTISGGSISSYYWSWIRQPPGKGLEWIGYIFYIGNTNYYPSLKSRVTISVDASKNQFSLKLSSVTAADTAVYYCARSYPYWYFDLWGRGTLVTVSS

SEQ ID NO: 926 - protein, 107 aa, synthetic construct
  REGION 1..107: note = Synthetic Construct
EIVMTQSPATLPVSPGERATLSCRASQSVSINLAWYQQKPGQAPRLLIYGVSTRATGIPARFSGSGSGTEFTLTISGLQSEDFAVYYCQQYNNWPRTFGQGTKVEIK

SEQ ID NO: 927 - protein, 116 aa, synthetic construct
  REGION 1..116: note = Synthetic Construct
EVQLVESGGGLVQPGGSLRLSCAASGFTFSRYWMHWVRQAPGKGLVWVSRINSDGSSSNYADSVKGRFTFSRDNAKNTLYLQMNSLRAEDTAVYFCVRDNWYFDYWGQGTLVTVSS

SEQ ID NO: 928 - protein, 110 aa, synthetic construct
  REGION 1..110: note = Synthetic Construct
QSVLTQPPSASGTPGQRVTISCSGSSSNIGSDYIYWYQQLPGTAPKLLIYSNHQRPSGVPDRFSGSKSGTSASLAISGLRSEDEADYYCAAWEDSLSGPIFGGGTKLTVL

SEQ ID NO: 929 - protein, 115 aa, synthetic construct
  REGION 1..115: note = Synthetic Construct
QVQLQESGPGLVKPSETLSLTCTVSGGSISSYYWSWIRQPPGKGLEWIGYIHYSGISNYNPSLKSRVAISLDTSNNHFSLKLTSLTAADTAVYFCARGSYWFDPWGQGTLVTVSS

SEQ ID NO: 930 - protein, 111 aa, synthetic construct
  REGION 1..111: note = Synthetic Construct
QLVLTQSPSASASLGASVKLTCTLSSGHSFYAIAWHQQQPEKGPRYLMQVNSNGSHNKGDGIPDRFSGSSSGAERYLTISSLQSEDEADYYCQSWDTDIWVFGGGTKLTVL

SEQ ID NO: 931 - protein, 115 aa, synthetic construct
  REGION 1..115: note = Synthetic Construct
KVQLVESGGGLVQPGGSLRLSCEASGFTFSNYWMHWVRQVPGEGLVWVSRLNNGGTSTRYADSVKSRFTISRDNAKNTLYLQMNSLRAEDTAVYYCVRDWYFDLWGRGTLVTVSS

SEQ ID NO: 932 - protein, 112 aa, synthetic construct
  REGION 1..112: note = Synthetic Construct
DIVMTQTPLSLPVTPGEPASISCRSSQSIVHSNANTYLEWYLQKPGQSPQLLIYKVSNRFSGVPDRFSGSGSGTDFTLKISRVEAEDVGVYYCFQGSHVPYTFGGGTKVEIK

SEQ ID NO: 933 - protein, 120 aa, synthetic construct
  REGION 1..120: note = Synthetic Construct
EVQLVESGGGLVQPGGSLRLSCAASGFTFSNYDMSWVRQAPGKGLEWVATISSGGSYTYYVDSVKGRFTISRDNAKNSLYLQMNSLRAEDTAVYYCARQELGSYYAMDYWGQGTTVTVSS

SEQ ID NO: 934 - protein, 107 aa, synthetic construct [sequence omitted]

SEQ ID NO: 935 - protein, 121 aa, synthetic construct
  REGION 1..121: note = Synthetic Construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARGSALHFFGVFDYWGQGTLVTVSS

SEQ ID NO: 936 - protein, 107 aa, synthetic construct
  REGION 1..107: note = Synthetic Construct
DIQMTQSPSSLSASVGDRVTITCRASQGISNYLAWYQQKPGKAPKLLIYAASSLQSGVPSRFSGSGSGTDFTLTISSLQPEDFATYYCQQSYSTPLTFGGGTKVEIK

SEQ ID NO: 937 - protein, 127 aa, synthetic construct
  REGION 1..127: note = Synthetic Construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAAGYYYDSSGYYHQPYYFDYWGQGTLVTVSS

SEQ ID NO: 938 - protein, 110 aa, synthetic construct
  REGION 1..110: note = Synthetic Construct
QSALTQPASVSGSPGQSITISCTGTTSDIGSYNLVSWYQQHPGKAPKLMIYEVSMSPSGVSNRFSGSKSGNTASLTISGLQAEDEADYYCSSYAGSSTVVFGGGTRLTVL

SEQ ID NO: 939 - protein, 118 aa, synthetic construct
  REGION 1..118: note = Synthetic Construct
QVQLQESGPGLVKPSETLSLTCTVSGGSISDYYWIWIRQPAGKGLEWIGRIYTTGGAKYNPSLKSRVTMSVDTSKNQFSLKLSSVTAADTAVYFCARDRNWEWNFDLWGRGTLVTVSS

SEQ ID NO: 940 - protein, 110 aa, synthetic construct; identical to SEQ ID NO: 938
  REGION 1..110: note = Synthetic Construct

SEQ ID NO: 941 - protein, 118 aa, synthetic construct
  REGION 1..118: note = Synthetic Construct
QVQLQESGPGLVKPSETLSLTCTVSGGSISDYYWSWIRQPAGKGLEWIGRIYTTGGAKYNPSLKSRVTMSVDTSKNQFSLKLSSVTAADTAVYYCARDRNWEWNFDLWGRGTLVTVSS

SEQ ID NO: 942 - protein, 112 aa, synthetic construct
  REGION 1..112: note = Synthetic Construct
QSVLTQPPSVSGAPGQRVTISCTGSSSNIGAGYNVHWYQQLPGTAPKLLIYGNSNRPSGVPDRFSGSKSGTSASLAITGLQAEDEADYYCQSYDSSLSGSAVFGGGTKLTVL

SEQ ID NO: 943 - protein, 118 aa, synthetic construct
  REGION 1..118: note = Synthetic Construct
QVQLQESGPGLVKPSETLSLTCTVSGGSMSSYFWSWIRQPPGKGLEWIGYMYYSGNTNYNPSLKSRVTISVDTSKNQFSLKLSSVTAADTAVYYCARSGNFYWNFDLWGRGTLVTVSS

SEQ ID NO: 944 - protein, 111 aa, synthetic construct
  REGION 1..111: note = Synthetic Construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGVVFGGGTKLTVL

SEQ ID NO: 945 - protein, 117 aa, synthetic construct
  REGION 1..117: note = Synthetic Construct
QVQLQESGPGLVKPSETLSLTCTVSGGSISSYYWSWIRQPPGKGLEWIGYIFYIGNTNYYPSLKSRVTISVDTSKNQFSLKLSSVTAADTAVYYCARSYPYWYFDLWGRGTLVTVSS

SEQ ID NO: 946 - protein, 107 aa, synthetic construct; identical to SEQ ID NO: 248
  REGION 1..107: note = Synthetic Construct

SEQ ID NO: 947 - protein, 121 aa, synthetic construct; identical to SEQ ID NO: 249
  REGION 1..121: note = Synthetic Construct

SEQ ID NO: 948 - protein, 107 aa, synthetic construct; identical to SEQ ID NO: 260
  REGION 1..107: note = Synthetic Construct

SEQ ID NO: 949 - protein, 119 aa, synthetic construct; identical to SEQ ID NO: 261
  REGION 1..119: note = Synthetic Construct

SEQ ID NO: 950 - protein, 107 aa, synthetic construct; identical to SEQ ID NO: 262
  REGION 1..107: note = Synthetic Construct

SEQ ID NO: 951 - protein, 127 aa, synthetic construct; identical to SEQ ID NO: 263
  REGION 1..127: note = Synthetic Construct

SEQ ID NO: 952 - protein, 107 aa, synthetic construct; identical to SEQ ID NO: 266
  REGION 1..107: note = Synthetic Construct

SEQ ID NO: 953 - protein, 121 aa, synthetic construct; identical to SEQ ID NO: 267
  REGION 1..121: note = Synthetic Construct

SEQ ID NO: 954 - protein, 110 aa, synthetic construct
  REGION 1..110: note = Synthetic Construct
QSALTQPASVSGSPGQSITISCTGTTSDIGSYNLVSWYQQHPGKAPKLMIYEVSMCPSGVSDRFSGSKSGSTASLTISGLQAEDEADYYCCSYAGSSTVVFGGGTRLTVL

SEQ ID NO: 955 - protein, 118 aa, synthetic construct; identical to SEQ ID NO: 915
  REGION 1..118: note = Synthetic Construct

SEQ ID NO: 956 - protein, 112 aa, synthetic construct; identical to SEQ ID NO: 916
  REGION 1..112: note = Synthetic Construct

SEQ ID NO: 957 - protein, 118 aa, synthetic construct; identical to SEQ ID NO: 917
  REGION 1..118: note = Synthetic Construct

SEQ ID NO: 958 - protein, 111 aa, synthetic construct; identical to SEQ ID NO: 924
  REGION 1..111: note = Synthetic Construct

SEQ ID NO: 959 - protein, 117 aa, synthetic construct; identical to SEQ ID NO: 925
  REGION 1..117: note = Synthetic Construct

SEQ ID NO: 960 - protein, 107 aa, synthetic construct [sequence omitted]

SEQ ID NO: 961 - protein, 121 aa, synthetic construct; identical to SEQ ID NO: 935
  REGION 1..121: note = Synthetic Construct

SEQ ID NO: 962 - protein, 107 aa, synthetic construct; identical to SEQ ID NO: 936
  REGION 1..107: note = Synthetic Construct

SEQ ID NO: 963 - protein, 127 aa, synthetic construct; identical to SEQ ID NO: 937
  REGION 1..127: note = Synthetic Construct

SEQ ID NO: 964 - protein, 110 aa, synthetic construct; identical to SEQ ID NO: 938
  REGION 1..110: note = Synthetic Construct

SEQ ID NO: 965 - protein, 118 aa, synthetic construct; identical to SEQ ID NO: 939
  REGION 1..118: note = Synthetic Construct

SEQ ID NO: 966 - protein, 110 aa, synthetic construct; identical to SEQ ID NO: 938
  REGION 1..110: note = Synthetic Construct

SEQ ID NO: 967 - protein, 118 aa, synthetic construct; identical to SEQ ID NO: 941
  REGION 1..118: note = Synthetic Construct

SEQ ID NO: 968 - protein, 112 aa, synthetic construct; identical to SEQ ID NO: 942
  REGION 1..112: note = Synthetic Construct

SEQ ID NO: 969 - protein, 118 aa, synthetic construct; identical to SEQ ID NO: 943
  REGION 1..118: note = Synthetic Construct

SEQ ID NO: 970 - protein, 111 aa, synthetic construct; identical to SEQ ID NO: 944
  REGION 1..111: note = Synthetic Construct

SEQ ID NO: 971 - protein, 117 aa, synthetic construct; identical to SEQ ID NO: 945
  REGION 1..117: note = Synthetic Construct

SEQ ID NO: 972-999 - skipped (28 entries)

SEQ ID NO: 1000 - protein, 217 aa, synthetic construct
  REGION 1..217: note = Synthetic Construct
QSVLTQPPSVSAAPGQKVTISCSGSSSNIGNNYVSWYQQFPGTAPKLLIYDNNKRPSGIPDRFSGSKSGTSATLGITGLQTGDEADYYCGTWDSSLNAGWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 1001 - protein, 221 aa, synthetic construct
  REGION 1..221: note = Synthetic Construct
QTVLTQPSSLSASPGASASLTCTLRSGINVGSYRIYWYQQKPGSPPQFLLRYKSDSDKQQGSGVPSRFSGSKDDSANAGILLISGLQSEDEADYYCMIWHSSASVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 1002 - protein, 216 aa, synthetic construct
  REGION 1..216: note = Synthetic Construct
QSVLTQSPSASGTPGQRVTISCSGSSSNIGSNTVHWYQQLPGTAPKLLMYGNYQRPSGVPARFSGSKSGTSASLAISGLQSEDEADYYCATWDDSLNGWVFGGGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 1003 - protein, 214 aa, synthetic construct
  REGION 1..214: note = Synthetic Construct
DIQMTQSPSAMSASVGDRVTITCRASQGISNYLAWFQQKPGKVPKRLIYAASSLQSGVPSRFSGSGSGTEFTLTISSLQPEDFATYYCLQHSFYPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1004 - protein, 217 aa, synthetic construct
  REGION 1..217: note = Synthetic Construct
QSVLTQPPSVSAAPGQKVTISCSGSSSNIGNNYVSWYQQFPGTAPKLLIYDNNKRPSGIPDRFSGSKSGTSATLGITGLQTGDEADYYCGTWDSSLSAGVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 1005 - protein, 219 aa, synthetic construct
  REGION 1..219: note = Synthetic Construct
DVVMTQSPLSLPVTLGQPASMSCRSSQSLAYSDGNTYLNWFQQRPGQSPRRLIYKVSNRDSGVPDRFSGSGSGTDFTLKISRVEAEDVGVYYCMQGTHWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1006 - protein, 215 aa, synthetic construct
  REGION 1..215: note = Synthetic Construct
QTVVTQEPSLTVSPGGTVTLTCASSTGAVTSGYYPNWFQQKPGQAPRALIYSTSNKHSWTPARFSGSLLGGKAALTLSGVQPEDEAEYYCLLYYDGAHLFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 1007 - protein, 216 aa, synthetic construct
  REGION 1..216: note = Synthetic Construct
QSALTQPASVSGSPGQSITISCTGTTSDIGSYNLVSWYQQHPGKAPKLMIYEVSMSPSGVSDRFSGSKSGSTASLTISGLQAEDEADYYCSSYAGSSTVVFGGGTRLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 1008 - protein, 218 aa, synthetic construct
  REGION 1..218: note = Synthetic Construct
QSVLTQPPSVSGAPGQRVTISCTGSSSNIGAGYNVHWYQQLPGTAPKVLISGNSNRPSGVPDRFSGSKSGTSASLAITGLQAEDEADYYCQSYDSSLSGSAVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 1009 - protein, 220 aa, synthetic construct
  REGION 1..220: note = Synthetic Construct
DIVMTQTPLSLPVTPGEPASISCRSSQSLLDSDDGNTYLDWYLQKPGQSPQLLIYTLSYRASGVPDRFSGSGSGTDFTLKVSRVEAEDVGVYYCMQRIEFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1010 - protein, 217 aa, synthetic construct
  REGION 1..217: note = Synthetic Construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDTSLSSGWLFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 1011 - protein, 214 aa, synthetic construct
  REGION 1..214: note = Synthetic Construct
DIQLTQSPSFLSASVGDKVTITCRASQGINSYLAWYQQKPGKAPKLLIYSASTLQGGVPSRFSGSGSGTEFTLTISNLQPADFATYCCQQLNSYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1012 - protein, 217 aa, synthetic construct
  REGION 1..217: note = Synthetic Construct
QSVLTQPPSVSAAPGQKVTISCSGSSSNIGNNYVSWYQQLPGTAPKLLISDNNKRPSGIPDRFSGSKSGTSATLGITGLQTGDEADYYCGTWDSSLSAGVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 1013 - protein, 214 aa, synthetic construct
  REGION 1..214: note = Synthetic Construct
EIVMTQSPATLPVSPGERATLSCRASQSVSINLAWYQQKPGQAPRLLIYGVSTRATGIPARFSGSGSGTEFTLTISGLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1014 - protein, 216 aa, synthetic construct
  REGION 1..216: note = Synthetic Construct
QSVLTQPPSASGTPGQRVTISCSGSSSNIGSDYIYWYQQLPGTAPKLLIYSNHQRPSGVPDRFSGSKSGTSASLAISGLRSEDEADYYCAAWEDSLSGPIFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 1015 - protein, 217 aa, synthetic construct
  REGION 1..217: note = Synthetic Construct
QLVLTQSPSASASLGASVKLTCTLSSGHSFYAIAWHQQQPEKGPRYLMQVNSNGSHNKGDGIPDRFSGSSSGAERYLTISSLQSEDEADYYCQSWDTDIWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 1016 - protein, 219 aa, synthetic construct
  REGION 1..219: note = Synthetic Construct
DIVMTQTPLSLPVTPGEPASISCRSSQSIVHSNANTYLEWYLQKPGQSPQLLIYKVSNRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1017 - protein, 214 aa, synthetic construct
  REGION 1..214: note = Synthetic Construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1018 - protein, 214 aa, synthetic construct
  REGION 1..214: note = Synthetic Construct
DIQMTQSPSSLSASVGDRVTITCRASQGISNYLAWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1019 - protein, 216 aa, synthetic construct
  REGION 1..216: note = Synthetic Construct
QSALTQPASVSGSPGQSITISCTGTTSDIGSYNLVSWYQQHPGKAPKLMIYEVSMSPSGVSNRFSGSKSGNTASLTISGLQAEDEADYYCSSYAGSSTVVFGGGTRLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 1020 - protein, 216 aa, synthetic construct; identical to SEQ ID NO: 1019
  REGION 1..216: note = Synthetic Construct

SEQ ID NO: 1021 - protein, 218 aa, synthetic construct
  REGION 1..218: note = Synthetic Construct
QSVLTQPPSVSGAPGQRVTISCTGSSSNIGAGYNVHWYQQLPGTAPKLLIYGNSNRPSGVPDRFSGSKSGTSASLAITGLQAEDEADYYCQSYDSSLSGSAVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 1022 - protein, 217 aa, synthetic construct
  REGION 1..217: note = Synthetic Construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 1023-1029 - skipped (7 entries)

SEQ ID NO: 1030 - protein, 461 aa, synthetic construct
  REGION 1..461: note = Synthetic Construct
QVQLQESGPGLVKPSETLSLTCTVSGGSISSYFWSWIRQPPGKGLEWIGYIYYSGNTNYNPSLKSRVTISVDMSKNQFSLRLSSVTAADTAVYYCARDFHHYDGSAYRWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1031 - protein, 457 aa, synthetic construct
  REGION 1..457: note = Synthetic Construct
QVQLQESGPGLVKPSETLSLTCTVSGGSISSNYWTWIRQSAGKGLEWIGRIHISESTNYNPSLKSRVTMSVDTSKNQFSLKLSSVTAADTAVYFCAREGEWKVLGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1032 - protein, 458 aa, synthetic construct
  REGION 1..458: note = Synthetic Construct
QVQLVQSGAEVKKPGASVKVSCKASGYTFTVYYIHWVRQAPGQGLEWMGWISPNSGGTNYAQKFQGRVTMTRDTSISTAYMELTRLRSDDTAIFYCARQTVVTPNWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1033 - protein, 457 aa, synthetic construct
  REGION 1..457: note = Synthetic Construct
QVQLQESGPGLVKPSETLSLTCTVSGGSISSYYWSWIRQPPGKGLEWIGYIYYSGNVNYNPSLKSRVTISVDASKNQFSLKLTSVTAADTAVYYCAIGGVGAQWDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1034 - protein, 456 aa, synthetic construct
  REGION 1..456: note = Synthetic Construct
QVQLQESGPGLVKPSATLSLTCTVSGGSISSYYWNWIRQPPGKGLEWIGYIYYSGNTNYNPSLKSRVTISVDMSKNQFSLKLNSVTAADTAVYYCARSESYGYWFFYLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1035 - protein, 456 aa, synthetic construct
  REGION 1..456: note = Synthetic Construct
QVQLQESGPGLVKPSETLSLTCTVSGGSISSYFWNWIRQPPGKGLEWIGYLYYIGTTNYNPSLKSRVTISVDTSKNQFSLKLNSVTAADTAVYYCARSGNYYNDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1036 - protein, 457 aa, synthetic construct
  REGION 1..457: note = Synthetic Construct
QVQLVQSGAEVKKPGASVKVSCKASGYTFTGYFIHWVRQAPGQGLEWMGWINPNSGGTNYAQKFQGRVTMTRDTSISTAYMELSRLRSDDTAVYYCARDTFYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1037 - protein, 455 aa, synthetic construct
  REGION 1..455: note = Synthetic Construct
QVQLQESGPGLVKPSETLSLTCTVSGGSISDYYWIWIRQPAGKGLEWIGRIYTTGGAKYNPSLKSRVTMSIDTSKNNFSLKLSSVTAADTAVYFCARDRNWEWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1038 - protein, 455 aa, synthetic construct
  REGION 1..455: note = Synthetic Construct
QVQLQESGPGLVKPSETLSLTCTVSGGSMSSYFWSWIRQPPERGLEWVGYMYYSGNTNYNPSLKSRVTTSIDTSKNQFSLKLTSVTAADTAVYYCARSGNFYWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1039 - protein, 456 aa, synthetic construct
  REGION 1..456: note = Synthetic Construct
EVQLVESGGGLVKPGGSLRLSCAVSGFTFSSYNMNWVRQAPGKGLEWVSSISISSSYIYYADSVKGRFTISRDNAKNSLYLQMNSLRAEDTAVYYCARDRGLDDAFDIWGQGTVVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1040 - protein, 454 aa, synthetic construct
  REGION 1..454: note = Synthetic Construct
QVHLQESGPGLVKPSETLSLTCTVSGGSIRSYYWSWIRQPAGKGLEWIGRIYTSGSTNYNPSLKSRVTMSVDTSKNRLSLKLSSVTAADTAVYYCAKENWGPSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1041 - protein, 455 aa, synthetic construct
  REGION 1..455: note = Synthetic Construct
QVQLVQSGAEAKKPGASVRVSCKTSGYTFTNYYIHWVRQAPGQGLEWMGIINPTLDSTSYAQKFQDRVTMTRDTSTNTDYMDLSSLRFEDTAVYYCARSAGFDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1042 - protein, 454 aa, synthetic construct
  REGION 1..454: note = Synthetic Construct
QVQLQESGPGLVKPSETLSLTCTISGGSISSYYWSWIRQPPGKGLEWIGYIFYIGNTNYYPSLKSRVTISVDASKNQFSLKLSSVTAADTAVYYCARSYPYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1043 - protein, 453 aa, synthetic construct
  REGION 1..453: note = Synthetic Construct
EVQLVESGGGLVQPGGSLRLSCAASGFTFSRYWMHWVRQAPGKGLVWVSRINSDGSSSNYADSVKGRFTFSRDNAKNTLYLQMNSLRAEDTAVYFCVRDNWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1044 - protein, 452 aa, synthetic construct
  REGION 1..452: note = Synthetic Construct
QVQLQESGPGLVKPSETLSLTCTVSGGSISSYYWSWIRQPPGKGLEWIGYIHYSGISNYNPSLKSRVAISLDTSNNHFSLKLTSLTAADTAVYFCARGSYWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1045 - protein, 452 aa, synthetic construct
  REGION 1..452: note = Synthetic Construct
KVQLVESGGGLVQPGGSLRLSCEASGFTFSNYWMHWVRQVPGEGLVWVSRLNNGGTSTRYADSVKSRFTISRDNAKNTLYLQMNSLRAEDTAVYYCVRDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1046 - protein, 457 aa, synthetic construct
  REGION 1..457: note = Synthetic Construct
EVQLVESGGGLVQPGGSLRLSCAASGFTFSNYDMSWVRQAPGKGLEWVATISSGGSYTYYVDSVKGRFTISRDNAKNSLYLQMNSLRAEDTAVYYCARQELGSYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1047 - protein, 458 aa, synthetic construct
  REGION 1..458: note = Synthetic Construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARGSALHFFGVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1048 - protein, 464 aa, synthetic construct
  REGION 1..464: note = Synthetic Construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAAGYYYDSSGYYHQPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1049 - protein, 455 aa, synthetic construct
  REGION 1..455: note = Synthetic Construct
QVQLQESGPGLVKPSETLSLTCTVSGGSISDYYWIWIRQPAGKGLEWIGRIYTTGGAKYNPSLKSRVTMSVDTSKNQFSLKLSSVTAADTAVYFCARDRNWEWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1050 - protein, 455 aa, synthetic construct
  REGION 1..455: note = Synthetic Construct
QVQLQESGPGLVKPSETLSLTCTVSGGSISDYYWSWIRQPAGKGLEWIGRIYTTGGAKYNPSLKSRVTMSVDTSKNQFSLKLSSVTAADTAVYYCARDRNWEWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1051 - protein, 455 aa, synthetic construct
  REGION 1..455: note = Synthetic Construct
QVQLQESGPGLVKPSETLSLTCTVSGGSMSSYFWSWIRQPPGKGLEWIGYMYYSGNTNYNPSLKSRVTISVDTSKNQFSLKLSSVTAADTAVYYCARSGNFYWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1052 - protein, 454 aa, synthetic construct
  REGION 1..454: note = Synthetic Construct
QVQLQESGPGLVKPSETLSLTCTVSGGSISSYYWSWIRQPPGKGLEWIGYIFYIGNTNYYPSLKSRVTISVDTSKNQFSLKLSSVTAADTAVYYCARSYPYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1053 - protein, 458 aa, synthetic construct
  REGION 1..458: note = Synthetic Construct
EVQLVQSGAEVKKPGSSVKVSCKASGYTFTGYYMHWVRQAPGQGLEWMGWINPNSGGTNYAQKFQGRVTMTRDTSISTAYMELRSLRSDDTAVYYCARGSALHFFGVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1054 - protein, 456 aa, synthetic construct
  REGION 1..456: note = Synthetic Construct
EVQLVQSGAEVKKPGASVKVSCKASGYTFTSYDINWVRQAPGQGLEWMGGIIPIFGTANYAQKFQGRVTITADESTSTAYMELRSLRSEDTAVYYCASTAVAGFGIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1055 - protein, 464 aa, synthetic construct
  REGION 1..464: note = Synthetic Construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAAGYYYDSSGYYHQPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1056 - protein, 458 aa, synthetic construct
  REGION 1..458: note = Synthetic Construct
EVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARATLQLWLPLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1057 - protein, 455 aa, synthetic construct
  REGION 1..455: note = Synthetic Construct
QVQLQESGPGLVKPSETLSLTCTVSGGSISDYYWIWIRQPAGKGLEWIGRIYTTGGAKYNPSLKSRVTMSIDTSKNNFSLKLSSVTAADTAVYFCARDRNWEWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1058 - protein, 455 aa, synthetic construct
  REGION 1..455: note = Synthetic Construct
QVQLQESGPGLVKPSETLSLTCTVSGGSMSSYFWSWIRQPPERGLEWVGYMYYSGNTNYNPSLKSRVTTSIDTSKNQFSLKLTSVTAADTAVYYCARSGNFYWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1059 - protein, 454 aa, synthetic construct
  REGION 1..454: note = Synthetic Construct
QVQLQESGPGLVKPSETLSLTCTISGGSISSYYWSWIRQPPGKGLEWIGYIFYIGNTNYYPSLKSRVTISVDASKNQFSLKLSSVTAADTAVYYCARSYPYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1060 - protein, 458 aa, synthetic construct
  REGION 1..458: note = Synthetic Construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARGSALHFFGVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1061 - protein, 464 aa, synthetic construct
  REGION 1..464: note = Synthetic Construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAAGYYYDSSGYYHQPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1062 - protein, 455 aa, synthetic construct
  REGION 1..455: note = Synthetic Construct
QVQLQESGPGLVKPSETLSLTCTVSGGSISDYYWIWIRQPAGKGLEWIGRIYTTGGAKYNPSLKSRVTMSVDTSKNQFSLKLSSVTAADTAVYFCARDRNWEWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1063 - protein, 455 aa, synthetic construct
  REGION 1..455: note = Synthetic Construct
QVQLQESGPGLVKPSETLSLTCTVSGGSISDYYWSWIRQPAGKGLEWIGRIYTTGGAKYNPSLKSRVTMSVDTSKNQFSLKLSSVTAADTAVYYCARDRNWEWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1064 - protein, 455 aa, synthetic construct
  REGION 1..455: note = Synthetic Construct
QVQLQESGPGLVKPSETLSLTCTVSGGSMSSYFWSWIRQPPGKGLEWIGYMYYSGNTNYNPSLKSRVTISVDTSKNQFSLKLSSVTAADTAVYYCARSGNFYWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1065 - protein, 454 aa, synthetic construct
  REGION 1..454: note = Synthetic Construct
QVQLQESGPGLVKPSETLSLTCTVSGGSISSYYWSWIRQPPGKGLEWIGYIFYIGNTNYYPSLKSRVTISVDTSKNQFSLKLSSVTAADTAVYYCARSYPYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 1066-1069 - skipped (4 entries)

SEQ ID NO: 1070 - protein, 450 aa, synthetic construct
  REGION 1..450: note = Synthetic Construct
EVQLVQSGAEVKKPGSSVKVSCKASGYTFTGYYMHWVRQAPGQGLEWMGWINPNSGGTNYAQKFQGRVTMTRDTSISTAYMELRSLRSDDTAVYYCARGSALHFFGVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1071 - protein, 452 aa, synthetic construct
  REGION 1..452: note = Synthetic Construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYSSSWYEPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1072 - protein, 447 aa, synthetic construct
  REGION 1..447: note = Synthetic Construct
EVQLVQSGGGLVKPGGSLRLSCAASGFTFSDYYMSWVRQAPGKGLEWVSAISGSGGSTYYADSVKGRFTISRDNSKNTLYLQMNSLRAEDTAVYYCARDEGYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1073 - protein, 457 aa, synthetic construct
  REGION 1..457: note = Synthetic Construct
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKSSYYDYVWGSYRYTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1074 - protein, 449 aa, synthetic construct
  REGION 1..449: note = Synthetic Construct
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDYGYNWNFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1075 - protein, 451 aa, synthetic construct
  REGION 1..451: note = Synthetic Construct
QVQLQESGPGLVKPSGTLSLTCTVSGGSISSGGYYWAWIRQPPGKGLEWIGSIYYTGSTYYNPSLKSRVTIYVDTSKNQFSLKLSSVTAADTAVYYCARDPFPEPPDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1076 - protein, 448 aa, synthetic construct
  REGION 1..448: note = Synthetic Construct
EVQLVQSGAEVKKPGASVKVSCKASGYTFTSYDINWVRQAPGQGLEWMGGIIPIFGTANYAQKFQGRVTITADESTSTAYMELRSLRSEDTAVYYCASTAVAGFGIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1077 - protein, 456 aa, synthetic construct
  REGION 1..456: note = Synthetic Construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAAGYYYDSSGYYHQPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1078 - protein, 448 aa, synthetic construct
  REGION 1..448: note = Synthetic Construct
EVQLVESGGGLVQPGGSLRLSCAASGFTFSDYYMSWIRQAPGKGLEWVSGISWNSGSIGYADSVKGRFTISRDNSKNTLYLQMNSLRAEDTAVYYCASSGSWYLPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1079 - protein, 450 aa, synthetic construct
  REGION 1..450: note = Synthetic Construct
EVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARATLQLWLPLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1080 - protein, 449 aa, synthetic construct
  REGION 1..449: note = Synthetic Construct
EVQLVQSGAEVKKPGSSVKVSCKASGGTFSSYAISWVRQAPGQGLEWMGWINPNSGGTNYAQKFQGRVTMTRDTSTSTAYMELSSLRSEDTAVYYCATGYYGDLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1081 - protein, 446 aa, synthetic construct
  REGION 1..446: note = Synthetic Construct
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1082 - protein, 453 aa, synthetic construct
  REGION 1..453: note = Synthetic Construct
QVQLQESGPGLVKPSETLSLTCTVSGGSISSYFWSWIRQPPGKGLEWIGYIYYSGNTNYNPSLKSRVTISVDMSKNQFSLRLSSVTAADTAVYYCARDFHHYDGSAYRWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1083 - protein, 449 aa, synthetic construct
  REGION 1..449: note = Synthetic Construct
QVQLQESGPGLVKPSETLSLTCTVSGGSISSNYWTWIRQSAGKGLEWIGRIHISESTNYNPSLKSRVTMSVDTSKNQFSLKLSSVTAADTAVYFCAREGEWKVLGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1084 - protein, 450 aa, synthetic construct
  REGION 1..450: note = Synthetic Construct
QVQLVQSGAEVKKPGASVKVSCKASGYTFTVYYIHWVRQAPGQGLEWMGWISPNSGGTNYAQKFQGRVTMTRDTSISTAYMELTRLRSDDTAIFYCARQTVVTPNWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1085 - protein, 449 aa, synthetic construct
  REGION 1..449: note = Synthetic Construct
QVQLQESGPGLVKPSETLSLTCTVSGGSISSYYWSWIRQPPGKGLEWIGYIYYSGNVNYNPSLKSRVTISVDASKNQFSLKLTSVTAADTAVYYCAIGGVGAQWDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1086 - protein, 448 aa, synthetic construct
  REGION 1..448: note = Synthetic Construct
QVQLQESGPGLVKPSATLSLTCTVSGGSISSYYWNWIRQPPGKGLEWIGYIYYSGNTNYNPSLKSRVTISVDMSKNQFSLKLNSVTAADTAVYYCARSESYGYWFFYLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1087 - protein, 448 aa, synthetic construct
  REGION 1..448: note = Synthetic Construct
QVQLQESGPGLVKPSETLSLTCTVSGGSISSYFWNWIRQPPGKGLEWIGYLYYIGTTNYNPSLKSRVTISVDTSKNQFSLKLNSVTAADTAVYYCARSGNYYNDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1088 - protein, 449 aa, synthetic construct
  REGION 1..449: note = Synthetic Construct
QVQLVQSGAEVKKPGASVKVSCKASGYTFTGYFIHWVRQAPGQGLEWMGWINPNSGGTNYAQKFQGRVTMTRDTSISTAYMELSRLRSDDTAVYYCARDTFYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1089 - protein, 447 aa, synthetic construct
  REGION 1..447: note = Synthetic Construct
QVQLQESGPGLVKPSETLSLTCTVSGGSISDYYWIWIRQPAGKGLEWIGRIYTTGGAKYNPSLKSRVTMSIDTSKNNFSLKLSSVTAADTAVYFCARDRNWEWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1090 - protein, 447 aa, synthetic construct
  REGION 1..447: note = Synthetic Construct
QVQLQESGPGLVKPSETLSLTCTVSGGSMSSYFWSWIRQPPERGLEWVGYMYYSGNTNYNPSLKSRVTTSIDTSKNQFSLKLTSVTAADTAVYYCARSGNFYWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1091 - protein, 448 aa, synthetic construct
  REGION 1..448: note = Synthetic Construct
EVQLVESGGGLVKPGGSLRLSCAVSGFTFSSYNMNWVRQAPGKGLEWVSSISISSSYIYYADSVKGRFTISRDNAKNSLYLQMNSLRAEDTAVYYCARDRGLDDAFDIWGQGTVVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1092 - protein, 446 aa, synthetic construct
  REGION 1..446: note = Synthetic Construct
QVHLQESGPGLVKPSETLSLTCTVSGGSIRSYYWSWIRQPAGKGLEWIGRIYTSGSTNYNPSLKSRVTMSVDTSKNRLSLKLSSVTAADTAVYYCAKENWGPSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1093 - protein, 447 aa, synthetic construct
  REGION 1..447: note = Synthetic Construct
QVQLVQSGAEAKKPGASVRVSCKTSGYTFTNYYIHWVRQAPGQGLEWMGIINPTLDSTSYAQKFQDRVTMTRDTSTNTDYMDLSSLRFEDTAVYYCARSAGFDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1094 - protein, 446 aa, synthetic construct
  REGION 1..446: note = Synthetic Construct
QVQLQESGPGLVKPSETLSLTCTISGGSISSYYWSWIRQPPGKGLEWIGYIFYIGNTNYYPSLKSRVTISVDASKNQFSLKLSSVTAADTAVYYCARSYPYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1095 - protein, 445 aa, synthetic construct
  REGION 1..445: note = Synthetic Construct
EVQLVESGGGLVQPGGSLRLSCAASGFTFSRYWMHWVRQAPGKGLVWVSRINSDGSSSNYADSVKGRFTFSRDNAKNTLYLQMNSLRAEDTAVYFCVRDNWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1096 - protein, 444 aa, synthetic construct
  REGION 1..444: note = Synthetic Construct
QVQLQESGPGLVKPSETLSLTCTVSGGSISSYYWSWIRQPPGKGLEWIGYIHYSGISNYNPSLKSRVAISLDTSNNHFSLKLTSLTAADTAVYFCARGSYWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1097 - protein, 444 aa, synthetic construct
  REGION 1..444: note = Synthetic Construct
KVQLVESGGGLVQPGGSLRLSCEASGFTFSNYWMHWVRQVPGEGLVWVSRLNNGGTSTRYADSVKSRFTISRDNAKNTLYLQMNSLRAEDTAVYYCVRDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1098 - protein, 449 aa, synthetic construct
  REGION 1..449: note = Synthetic Construct
EVQLVESGGGLVQPGGSLRLSCAASGFTFSNYDMSWVRQAPGKGLEWVATISSGGSYTYYVDSVKGRFTISRDNAKNSLYLQMNSLRAEDTAVYYCARQELGSYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1099 - protein, 450 aa, synthetic construct
  REGION 1..450: note = Synthetic Construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARGSALHFFGVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1100 - protein, 456 aa, synthetic construct
  REGION 1..456: note = Synthetic Construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAAGYYYDSSGYYHQPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1101 - protein, 447 aa, synthetic construct
  REGION 1..447: note = Synthetic Construct
QVQLQESGPGLVKPSETLSLTCTVSGGSISDYYWIWIRQPAGKGLEWIGRIYTTGGAKYNPSLKSRVTMSVDTSKNQFSLKLSSVTAADTAVYFCARDRNWEWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1102 - protein, 447 aa, synthetic construct
  REGION 1..447: note = Synthetic Construct
QVQLQESGPGLVKPSETLSLTCTVSGGSISDYYWSWIRQPAGKGLEWIGRIYTTGGAKYNPSLKSRVTMSVDTSKNQFSLKLSSVTAADTAVYYCARDRNWEWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1103 - protein, 447 aa, synthetic construct
  REGION 1..447: note = Synthetic Construct
QVQLQESGPGLVKPSETLSLTCTVSGGSMSSYFWSWIRQPPGKGLEWIGYMYYSGNTNYNPSLKSRVTISVDTSKNQFSLKLSSVTAADTAVYYCARSGNFYWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1104 - protein, 446 aa, synthetic construct
  REGION 1..446: note = Synthetic Construct
QVQLQESGPGLVKPSETLSLTCTVSGGSISSYYWSWIRQPPGKGLEWIGYIFYIGNTNYYPSLKSRVTISVDTSKNQFSLKLSSVTAADTAVYYCARSYPYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1105 - protein, 450 aa, synthetic construct
  REGION 1..450: note = Synthetic Construct
EVQLVQSGAEVKKPGSSVKVSCKASGYTFTGYYMHWVRQAPGQGLEWMGWINPNSGGTNYAQKFQGRVTMTRDTSISTAYMELRSLRSDDTAVYYCARGSALHFFGVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1106 - protein, 448 aa, synthetic construct
  REGION 1..448: note = Synthetic Construct
EVQLVQSGAEVKKPGASVKVSCKASGYTFTSYDINWVRQAPGQGLEWMGGIIPIFGTANYAQKFQGRVTITADESTSTAYMELRSLRSEDTAVYYCASTAVAGFGIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1107 - protein, 456 aa, synthetic construct
  REGION 1..456: note = Synthetic Construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAAGYYYDSSGYYHQPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1108 - protein, 450 aa, synthetic construct
  REGION 1..450: note = Synthetic Construct
EVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARATLQLWLPLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1109 - protein, 447 aa, synthetic construct
  REGION 1..447: note = Synthetic Construct
QVQLQESGPGLVKPSETLSLTCTVSGGSISDYYWIWIRQPAGKGLEWIGRIYTTGGAKYNPSLKSRVTMSIDTSKNNFSLKLSSVTAADTAVYFCARDRNWEWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1110 - protein, 447 aa, synthetic construct
  REGION 1..447: note = Synthetic Construct
QVQLQESGPGLVKPSETLSLTCTVSGGSMSSYFWSWIRQPPERGLEWVGYMYYSGNTNYNPSLKSRVTTSIDTSKNQFSLKLTSVTAADTAVYYCARSGNFYWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1111 - protein, 446 aa, synthetic construct
  REGION 1..446: note = Synthetic Construct
QVQLQESGPGLVKPSETLSLTCTISGGSISSYYWSWIRQPPGKGLEWIGYIFYIGNTNYYPSLKSRVTISVDASKNQFSLKLSSVTAADTAVYYCARSYPYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1112 - protein, 450 aa, synthetic construct
  REGION 1..450: note = Synthetic Construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARGSALHFFGVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1113 - protein, 456 aa, synthetic construct
  REGION 1..456: note = Synthetic Construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAAGYYYDSSGYYHQPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1114 - protein, 447 aa, synthetic construct
  REGION 1..447: note = Synthetic Construct
QVQLQESGPGLVKPSETLSLTCTVSGGSISDYYWIWIRQPAGKGLEWIGRIYTTGGAKYNPSLKSRVTMSVDTSKNQFSLKLSSVTAADTAVYFCARDRNWEWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1115 - protein, 447 aa, synthetic construct
  REGION 1..447: note = Synthetic Construct
QVQLQESGPGLVKPSETLSLTCTVSGGSISDYYWSWIRQPAGKGLEWIGRIYTTGGAKYNPSLKSRVTMSVDTSKNQFSLKLSSVTAADTAVYYCARDRNWEWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1116 - protein, 447 aa, synthetic construct
  REGION 1..447: note = Synthetic Construct
QVQLQESGPGLVKPSETLSLTCTVSGGSMSSYFWSWIRQPPGKGLEWIGYMYYSGNTNYNPSLKSRVTISVDTSKNQFSLKLSSVTAADTAVYYCARSGNFYWNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1117 - protein, 446 aa, synthetic construct
  REGION 1..446: note = Synthetic Construct
QVQLQESGPGLVKPSETLSLTCTVSGGSISSYYWSWIRQPPGKGLEWIGYIFYIGNTNYYPSLKSRVTISVDTSKNQFSLKLSSVTAADTAVYYCARSYPYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG